SEQUENCE LISTING (1 sequence)

SEQ ID NO: 1 - protein, 766 aa, Homo sapiens [sequence omitted]

(19) United States
(12) Patent Application Publication
(10) Pub. No.: US 2024/0317752 A1
(43) Pub. Date: Sep. 26, 2024

(54) 6-SUBSTITUTED-9H-PURINE DERIVATIVES AND RELATED USES

(71) Applicant: Black Diamond Therapeutics, Inc.

(72) Inventors: Pui Yee NG, Lexington, MA (US); Ivan JEWETT, Boston, MA (US); Matthew C. LUCAS, Lexington, MA (US); Fernando PADILLA, Arlington, MA (US); Istvan J. ENYEDY, Milton, MA (US)

(73) Assignee: Black Diamond Therapeutics, Inc., Cambridge, MA (US)

(21) Appl. No.: 18/276,467

(22) PCT Filed: Feb. 18, 2022
(86) PCT No.: PCT/US2022/016987

§ 371 Date: Aug. 9, 2023

Related U.S. Application Data
(60) Provisional application No. 63/242,843, filed on Sep. 10, 2021.
(60) Provisional application No. 63/150,927, filed on Feb. 18, 2021.

(51) Int. Cl. C07D 473/40; A61K 31/52; A61K 31/551; A61K 31/553; A61P 35/00; C07D 519/00

(52) U.S. Cl. CPC C07D 473/40; A61K 31/52; A61K 31/551; A61K 31/553; A61P 35/00; C07D 519/00

(57) ABSTRACT

The present disclosure relates compounds of Formula (I′):

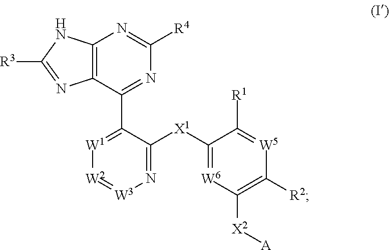

and pharmaceutically acceptable salts and stereoisomers thereof. The present disclosure also relates to methods of preparing the compounds, compositions comprising the compounds, and methods of using the compounds, e.g., in the treatment of cancer.

### RELATED APPLICATIONS

The application claims priority to, and the benefit of, U.S. Provisional Application No. 63/150,927, filed on Feb. 18, 2021, and 63/242,843, filed Sep. 10, 2021, the contents of each of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Feb. 17, 2022 is named “ASET-014_001 WO_SeqList” and is about 6,904 bytes in size.

### BACKGROUND

Specific mutations in the human gene BRAF, which encodes for the protein B-Raf, are known to drive oncogenic activity in a variety of different cancers. Targeted inactivation of mutant B-Raf proteins by the administration of protein kinase inhibitors has been used to treat a number of different cancers in patients. However, there are subsets of patients administered these treatments that either fail to respond, eventually relapse or experience secondary lesions/pathway rebound. More specifically, many of the existing B-Raf-targeting kinase inhibitors either exhibit low specificity for B-Raf, leading to undesirable off-target effects, or only target a specific subset of BRAF/B-Raf mutation(s). Thus, there is a long-felt need in the art for new therapies that target specific oncogenic forms of B-Raf produced by mutations or alterations of the BRAF gene. The present disclosure provides compositions and methods for preventing or treating cancer in patients with oncogenic mutations in the BRAF gene and B-Raf protein.

### SUMMARY

In some aspects, the present disclosure provides a compound of Formula (I′):

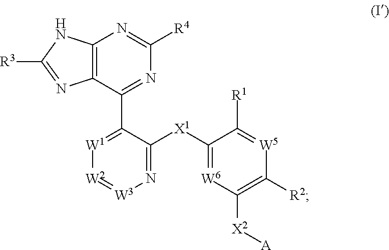

or a pharmaceutically acceptable salt or stereoisomer thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, C₂-C₆ alkynyl, or C₁-C₆ alkoxyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
R² is H, halogen, C₁-C₆ alkyl, C₂-C₆ alkenyl, or C₂-C₆ alkynyl;
R³ is H, halogen, C₁-C₆ alkyl, C₂-C₆ alkenyl, C₂-C₆ alkynyl, or C₁-C₆ alkoxyl, wherein the C₁-C₆ alkoxyl is optionally substituted with one or more OH, NH₂, NH(C₁-C₆ alkyl), or N(C₁-C₆ alkyl)₂;
R⁴ is H, halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is absent, C₂-C₆ alkynyl, —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A};
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH(C₁-C₆ alkyl), or N(C₁-C₆ alkyl)₂ is optionally substituted with one or more R^{A1}; and
each R^{A1} independently is halogen, cyano, OH, NH₂, NH(C₁-C₆ alkyl), N(C₁-C₆ alkyl)₂, C₁-C₆ alkyl, C₂-C₆ alkenyl, or C₂-C₆ alkynyl.

In some aspects, the present disclosure provides a compound of Formula (I):

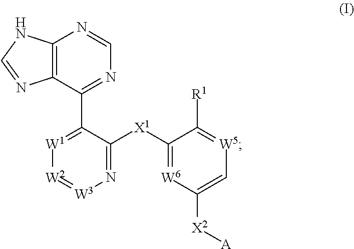

or a pharmaceutically acceptable salt or stereoisomer thereof, wherein: W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, or C₂-C₆ alkynyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is absent, C₂-C₆ alkynyl, —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}, and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

In some aspects, the present disclosure provides an isotopic derivative of a compound described herein.

In some aspects, the present disclosure provides a method of preparing a compound described herein.

In some aspects, the present disclosure provides a pharmaceutical composition comprising a compound described herein and one or more pharmaceutically acceptable carriers or excipients.

In some aspects, the present disclosure provides a method of treating or preventing cancer in a subject, the method comprising administering to the subject at least one therapeutically effective amount of a compound described herein.

In some aspects, the present disclosure provides a compound described herein for treating or preventing cancer in a subject.

In some aspects, the present disclosure provides a use of a compound described herein in the manufacture of a medicament for treating or preventing cancer in a subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

### Compounds of the Present Disclosure

In some aspects, the present disclosure provides a compound of Formula (I′):

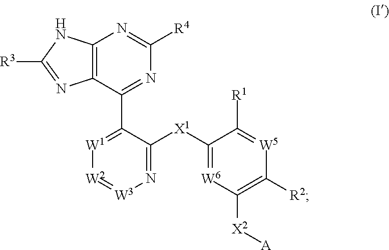

or a pharmaceutically acceptable salt or stereoisomer thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, C₂-C₆ alkynyl, or C₁-C₆ alkoxyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
R² is H, halogen, C₁-C₆ alkyl, C₂-C₆ alkenyl, or C₂-C₆ alkynyl;
R³ is H, halogen, C₁-C₆ alkyl, C₂-C₆ alkenyl, C₂-C₆ alkynyl, or C₁-C₆ alkoxyl, wherein the C₁-C₆ alkoxyl is optionally substituted with one or more OH, NH₂, NH(C₁-C₆ alkyl), or N(C₁-C₆ alkyl)₂;
R⁴ is H, halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is absent, C₂-C₆ alkynyl, —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A};
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH(C₁-C₆ alkyl), or N(C₁-C₆ alkyl)₂ is optionally substituted with one or more R^{A1}; and
each R^{A1} independently is halogen, cyano, OH, NH₂, NH(C₁-C₆ alkyl), N(C₁-C₆ alkyl)₂; C₁-C₆ alkyl, C₂-C₆ alkenyl, or C₂-C₆ alkynyl.

In some aspects, the present disclosure provides a compound of Formula (I′) or a pharmaceutically acceptable salt or stereoisomer thereof, wherein:
W¹ is N or CR^{W1};
R^{W1} is H, halogen, or C₁-C₆ alkyl;
W² is CR^{W2};
R^{W2} is H or halogen;
W³ is N or CR^{W3};
R^{W3} is H or C₁-C₆ alkoxyl;
X¹ is NR^{X1};
R^{X1} is H;
R¹ is C₁-C₆ alkyl;
R² is H or C₁-C₆ alkyl;
R³ is H, C₁-C₆ alkyl, or C₁-C₆ alkoxyl, wherein the C₁-C₆ alkoxyl is optionally substituted with one or more OH or N(C₁-C₆ alkyl)₂;
R⁴ is H, halogen, or C₁-C₆ alkyl;
W⁵ is CR^{W5};
R^{W5} is H;
W⁶ is CR^{W6};
R^{W6} is H;
X² is —NR^{X2}C(O)—* or —NR^{X2}C(O)CH₂—*, wherein * denotes attachment to A;
R^{X2} is H;
A is C₃-C₈ cycloalkyl, 3- to 8-membered heterocycloalkyl, or 5- to 9-membered heteroaryl, wherein the C₃-C₈ cycloalkyl, 3- to 8-membered heterocycloalkyl, or 5- to 9-membered heteroaryl is optionally substituted with one or more R^{A};
each R^{A} independently is halogen, C₁-C₆ alkyl, C₁-C₆ alkoxyl, —O-(3- to 8-membered heterocycloalkyl), NH₂, NH(C₁-C₆ alkyl), or N(C₁-C₆ alkyl)₂, wherein the C₁-C₆ alkyl, C₁-C₆ alkoxyl, or —O-(3- to 8-membered heterocycloalkyl) is optionally substituted with one or more R^{A1}; and
each R^{A1} independently is halogen, cyano, OH, N(C₁-C₆ alkyl)₂, or C₁-C₆ alkyl.

In some aspects, the present disclosure provides a compound of Formula (I):

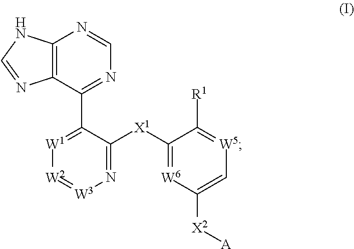

or a pharmaceutically acceptable salt or stereoisomer thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, or C₂-C₆ alkynyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is absent, C₂-C₆ alkynyl, —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}; and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

In some aspects, the present disclosure provides a compound of Formula (I):

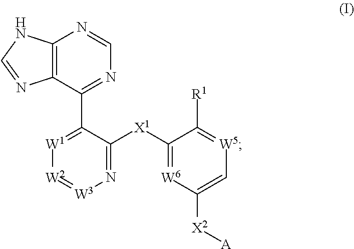

or a pharmaceutically acceptable salt or stereoisomer thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, or C₂-C₆ alkynyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}, and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

In some aspects, the present disclosure provides a compound of Formula (I):

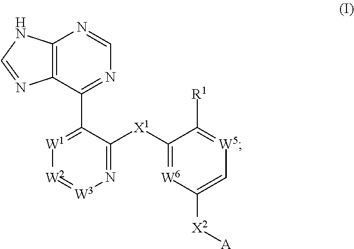

or a pharmaceutically acceptable salt or stereoisomer thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, or C₂-C₆ alkynyl;
X¹ is NR^{X1};
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}; and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

In some aspects, the present disclosure provides a compound of Formula (I):

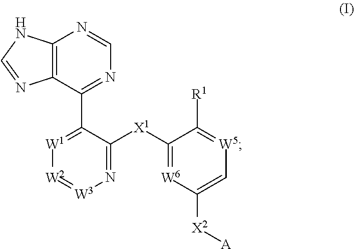

or a pharmaceutically acceptable salt or stereoisomer thereof, wherein:
W¹ is CR^{W1};
R^{W1} is H, halogen, C₁-C₆ alkyl, C₂-C₆ alkenyl, or C₂-C₆ alkynyl;
W² is CR^{W2};
R^{W2} is H, halogen, C₁-C₆ alkyl, C₂-C₆ alkenyl, or C₂-C₆ alkynyl;
W³ is CR^{W3};
R^{W3} is H, halogen, C₁-C₆ alkyl, C₂-C₆ alkenyl, or C₂-C₆ alkynyl;
X¹ is NR^{X1};
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is —NR^{X2}C(O)—*, —C(O)NR^{X2}—*, —NR^{X2}C(O)CH₂—*, or —C(O)NR^{X2}CH₂—*, wherein denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}, and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.
   Variables W¹, R^{W1}, W², R^{W2}, W³, and R^{W3}

In some embodiments, W¹ is N.

In some embodiments, W¹ is CR^{W1}.

In some embodiments, W¹ is CH.

In some embodiments, R^{W1} is H.

In some embodiments, R^{W1} is halogen, C₁-C₆ alkyl, C₂-C₆ alkenyl, or C₂-C₆ alkynyl.

In some embodiments, R^{W1} is halogen (e.g., F or Cl).

In some embodiments, R^{W1} is C₁-C₆ alkyl (e.g., CH₃).

In some embodiments, R^{W1} is C₂-C₆ alkenyl or C₂-C₆ alkynyl.

In some embodiments, W² is N.

In some embodiments, W² is CR^{W2}.

In some embodiments, W² is CH.

In some embodiments, R^{W2} is H.

In some embodiments, R^{W2} is halogen, C₁-C₆ alkyl, C₂-C₆ alkenyl, or C₂-C₆ alkynyl.

In some embodiments, R^{W2} is halogen (e.g., F or Cl).

In some embodiments, R^{W2} is C₁-C₆ alkyl (e.g., CH₃).

In some embodiments, R^{W2} is C₂-C₆ alkenyl or C₂-C₆ alkynyl.

In some embodiments, W³ is N.

In some embodiments, W³ is CR^{W3}.

In some embodiments, W³ is CH.

In some embodiments, R^{W3} is H.

In some embodiments, R^{W3} is halogen, C₁-C₆ alkyl, C₂-C₆ alkenyl, or C₂-C₆ alkynyl.

In some embodiments, R^{W3} is halogen, C₁-C₆ alkyl, C₂-C₆ alkenyl, C₂-C₆ alkynyl, or C₁-C₆ alkoxyl.

In some embodiments, R^{W3} is halogen (e.g., F or Cl).

In some embodiments, R^{W3} is C₁-C₆ alkyl (e.g., CH₃).

In some embodiments, R^{W3} is C₂-C₆ alkenyl or C₂-C₆ alkynyl.

In some embodiments, R^{W3} is C₁-C₆ alkoxyl.

In some embodiments, W¹, W², and W³ each are N.

In some embodiments, W¹ is CR^{W1}, W² is N, and W³ is N.

In some embodiments, W¹ is N, W² is N, and W³ is CR^{W3}.

In some embodiments, W¹ is N, W² is CR^{W2}, and W³ is N.

In some embodiments, W¹ is CR^{W1}, W² is CR^{W2}, and W³ is N.

In some embodiments, W¹ is CR^{W1}, W² is N, and W³ is CR^{W3}.

In some embodiments, W¹ is N, W² is CR^{W2}, and W³ is CR^{W3}.

In some embodiments, W¹ is CR^{W1}, W² is CR^{W2}, and W³ is CR^{W3}.

In some embodiments, W¹, W², and W³ each are CH.

Variables X¹ and R^{X1}

In some embodiments, X¹ is NR^{X1}.

In some embodiments, X¹ is NH.

In some embodiments, X¹ is O or S.

In some embodiments, X¹ is O.

In some embodiments, X¹ is S.

In some embodiments, R^{X1} is H

In some embodiments, R^{X1} is C₁-C₆ alkyl (e.g., CH₃).

In some embodiments, R^{X1} is C₂-C₆ alkenyl or C₂-C₆ alkynyl.

Variables R¹, R², R³, and R⁴

In some embodiments, R¹ is halogen.

In some embodiments, R¹ is F or Cl.

In some embodiments, R¹ is F.

In some embodiments, R¹ is C₁.

In some embodiments, R¹ is C₁-C₆ alkyl, C₂-C₆ alkenyl, or C₂-C₆ alkynyl.

In some embodiments, R¹ is C₁-C₆ alkyl.

In some embodiments, R¹ is CH₃.

In some embodiments, R¹ is C₂-C₆ alkenyl or C₂-C₆ alkynyl.

In some embodiments, R² is H, halogen, C₁-C₆ alkyl, C₂-C₆ alkenyl, or C₂-C₆ alkynyl.

In some embodiments, R² is H.

In some embodiments, R² is halogen, C₁-C₆ alkyl, C₂-C₆ alkenyl, or C₂-C₆ alkynyl.

In some embodiments, R² is C₁-C₆ alkyl (e.g., CH₃).

In some embodiments, R³ is H, halogen, C₁-C₆ alkyl, C₂-C₆ alkenyl, C₂-C₆ alkynyl, or C₁-C₆ alkoxyl, wherein the C₁-C₆ alkoxyl is optionally substituted with one or more OH, NH₂, NH(C₁-C₆ alkyl), or N(C₁-C₆ alkyl)₂.

In some embodiments, R³ is H, halogen, C₁-C₆ alkyl, C₂-C₆ alkenyl, C₂-C₆ alkynyl, or C₁-C₆ alkoxyl.

In some embodiments, R³ is H.

In some embodiments, R³ is halogen, C₁-C₆ alkyl, C₂-C₆ alkenyl, C₂-C₆ alkynyl, or C₁-C₆ alkoxyl, wherein the C₁-C₆ alkoxyl is optionally substituted with one or more OH, NH₂, NH(C₁-C₆ alkyl), or N(C₁-C₆ alkyl)₂.

In some embodiments, R³ is halogen, C₁-C₆ alkyl, C₂-C₆ alkenyl, C₂-C₆ alkynyl, or C₁-C₆ alkoxyl.

In some embodiments, R³ is C₁-C₆ alkyl (e.g., CH₃).

In some embodiments, R³ is CH₃.

In some embodiments, R³ is C₁-C₆ alkoxyl.

In some embodiments, R³ is —OCH₃.

In some embodiments, R³ is C₁-C₆ alkoxyl, optionally substituted with one or more OH, NH₂, NH(C₁-C₆ alkyl), or N(C₁-C₆ alkyl)₂.

In some embodiments, R³ is C₁-C₆ alkoxyl, substituted with one or more OH, NH₂, NH(C₁-C₆ alkyl), or N(C₁-C₆ alkyl)₂.

In some embodiments, R³ is C₁-C₆ alkoxyl, substituted with OH.

In some embodiments, R³ is C₁-C₆ alkoxyl, substituted with N(C₁-C₆ alkyl)₂.

In some embodiments, R⁴ is H, halogen, C₁-C₆ alkyl, C₂-C₆ alkenyl, or C₂-C₆ alkynyl.

In some embodiments, R⁴ is H.

In some embodiments, R⁴ is halogen, C₁-C₆ alkyl, C₂-C₆ alkenyl, or C₂-C₆ alkynyl.

In some embodiments, R⁴ is halogen.

In some embodiments, R⁴ is F or Cl.

In some embodiments, R⁴ is F.

In some embodiments, R⁴ is Cl.

In some embodiments, R⁴ is C₁-C₆ alkyl (e.g., CH₃).

In some embodiments, R⁴ is CH₃.

Variables W⁵, R^{W5}, W⁶, and R^{W6}

In some embodiments, W⁵ is N.

In some embodiments, W⁵ is CR^{W5}.

In some embodiments, W⁵ is CH.

In some embodiments, R^{W5} is H.

In some embodiments, R^{W5} is halogen, C₁-C₆ alkyl, C₂-C₆ alkenyl, or C₂-C₆ alkynyl.

In some embodiments, R^{W5} is halogen (e.g., F or Cl).

In some embodiments, R^{W5} is C₁-C₆ alkyl (e.g., CH₃).

In some embodiments, R^{W5} is C₂-C₆ alkenyl or C₂-C₆ alkynyl.

In some embodiments, W⁶ is N.

In some embodiments, W⁶ is CR^{W6.}

In some embodiments, W⁶ is CH.

In some embodiments, R^{W6} is H.

In some embodiments, R^{W6} is halogen, C₁-C₆ alkyl, C₂-C₆ alkenyl, or C₂-C₆ alkynyl.

In some embodiments, R^{W6} is halogen (e.g., F or Cl).

In some embodiments, R^{W6} is C₁-C₆ alkyl (e.g., CH₃).

In some embodiments, R^{W6} is C₂-C₆ alkenyl or C₂-C₆ alkynyl.

Variables X² and R^{X2}

In some embodiments, X² is absent.

In some embodiments, X² is C₂-C₆ alkynyl.

In some embodiments, X² is —NR^{X2}C(O)—*, —C(O)NR^{X2}—*, —NR^{X2}C(O)CH₂—*, or —C(O)NR^{X2}CH₂—*, wherein * denotes attachment to A.

In some embodiments, X² is —NR^{X2}C(O)—* or —C(O)NR^{X2}—*.

In some embodiments, X² is —NHC(O)—* or —C(O)NH—*.

In some embodiments, X² is —NR^{X2}C(O)—*.

In some embodiments, X² is —NHC(O)—*.

In some embodiments, X² is —C(O)NR^{X2}—*.

In some embodiments, X² is —C(O)NH—*.

In some embodiments, X² is —NR^{X2}C(O)CH₂—* or —C(O)NR^{X2}CH₂—*.

In some embodiments, X² is —NHC(O)CH₂—* or —C(O)NHCH₂—*.

In some embodiments, X² is —NR^{X2}C(O)CH₂—*.

In some embodiments, X² is —NHC(O)CH₂—*.

In some embodiments, X² is —C(O)NR^{X2}CH₂—*.

In some embodiments, X² is —C(O)NHCH₂—*.

In some embodiments, R^{X2} is H.

In some embodiments, R^{X2} is C₁-C₆ alkyl, C₂-C₆ alkenyl, or C₂-C₆ alkynyl.

In some embodiments, R^{X2} is C₁-C₆ alkyl (e.g., CH₃).

In some embodiments, R^{X2} is C₂-C₆ alkenyl or C₂-C₆ alkynyl.

Variables A, R^{A} and R^{A1}

In some embodiments, A is C₃-C₈ cycloalkyl or 3- to 8-membered heterocycloalkyl, wherein the C₃-C₈ cycloalkyl or 3- to 8-membered heterocycloalkyl is optionally substituted with one or more R^{A}.

In some embodiments, A is C₃-C₈ cycloalkyl or 3- to 8-membered heterocycloalkyl.

In some embodiments, A is C₃-C₈ cycloalkyl optionally substituted with one or more R^{A}. In some embodiments, A is C₃-C₈ cycloalkyl.

In some embodiments, A is C₃-C₈ cycloalkyl substituted with one or more R^{A}.

In some embodiments, A is 3- to 8-membered heterocycloalkyl optionally substituted with one or more R^{A}.

In some embodiments, A is 3- to 8-membered heterocycloalkyl.

In some embodiments, A is 3- to 8-membered heterocycloalkyl substituted with one or more R^{A}.

In some embodiments, A is C₆-C₁₀ aryl or 5- to 9-membered heteroaryl, wherein the C₆-C₁₀ aryl or 5- to 9-membered heteroaryl is optionally substituted with one or more R^{A}. In some embodiments, A is C₆-C₁₀ aryl or 5- to 9-membered heteroaryl.

In some embodiments, A is C₆-C₁₀ aryl optionally substituted with one or more R^{A}.

In some embodiments, A is C₆-C₁₀ aryl.

In some embodiments, A is C₆-C₁₀ aryl substituted with one or more R^{A}.

In some embodiments, A is phenyl optionally substituted with one or more R^{A}.

In some embodiments, A is phenyl.

In some embodiments, A is phenyl substituted with one or more R^{A}.

In some embodiments, A is

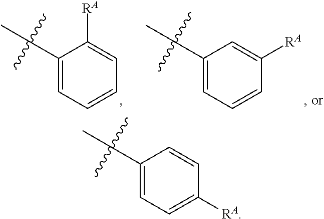

In some embodiments, A is

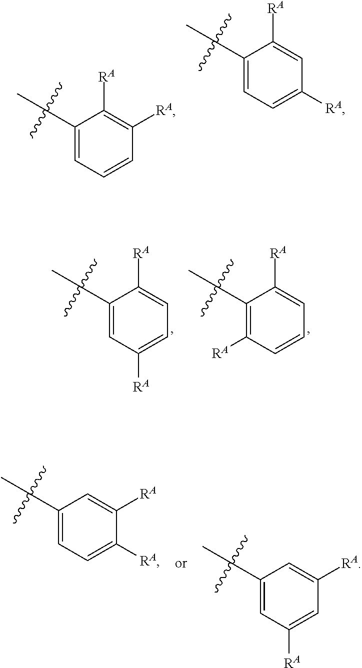

In some embodiments, A is 5- to 9-membered heteroaryl optionally substituted with one or more R^{A}.

In some embodiments, A is 5- to 9-membered heteroaryl.

In some embodiments, A is 5- to 9-membered heteroaryl substituted with one or more R^{A}.

In some embodiments, A is 6-membered heteroaryl optionally substituted with one or more R^{A}.

In some embodiments, A is 6-membered heteroaryl.

In some embodiments, A is 6-membered heteroaryl substituted with one or more R^{A}.

In some embodiments, A is pyridinyl optionally substituted with one or more R^{A}.

In some embodiments, A is pyridinyl.

In some embodiments, A is

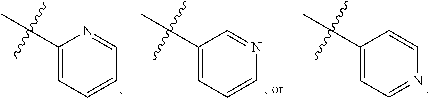 [SMILES: *c1ccccn1 ; *c1cccnc1 ; *c1ccncc1]

In some embodiments, A is pyridinyl substituted with one or more R^{A}.

In some embodiments, A is

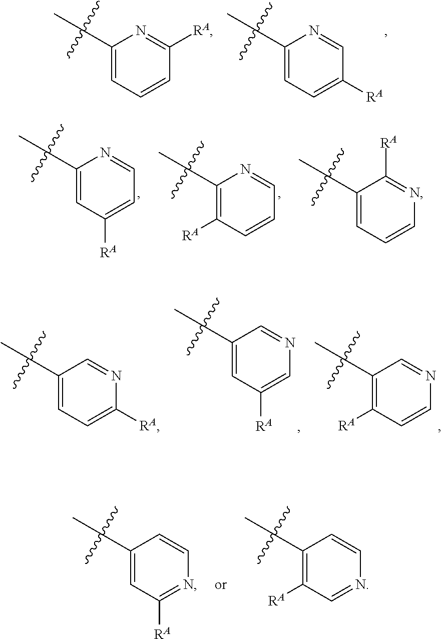

In some embodiments, each R^{A} independently is halogen or cyano.

In some embodiments, each R^{A} independently is halogen.

In some embodiments, each R^{A} independently is F, Cl, or Br.

In some embodiments, each R^{A} independently is F.

In some embodiments, each R^{A} independently is C₁.

In some embodiments, each R^{A} independently is Br.

In some embodiments, each R^{A} independently is cyano.

In some embodiments, each R^{A} independently is C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

In some embodiments, each R^{A} independently is C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH(C₁-C₆ alkyl), or N(C₁-C₆ alkyl)₂ is optionally substituted with one or more R^{A1}.

In some embodiments, each R^{A} independently is C₁-C₆ alkyl, C₂-C₆ alkenyl, or C₂-C₆ alkynyl, wherein the C₁-C₆ alkyl, C₂-C₆ alkenyl, C₂-C₆ alkynyl, or C₁-C₆ alkoxyl is optionally substituted with one or more halogen or cyano.

In some embodiments, each R^{A} independently is C₁-C₆ alkyl, C₂-C₆ alkenyl, or C₂-C₆ alkynyl, wherein the C₁-C₆ alkyl, C₂-C₆ alkenyl, C₂-C₆ alkynyl, or C₁-C₆ alkoxyl is optionally substituted with one or more R^{A1}.

In some embodiments, each R^{A} independently is C₁-C₆ alkyl (e.g., methyl, ethyl, or propyl) optionally substituted with one or more halogen or cyano.

In some embodiments, each R^{A} independently is C₁-C₆ alkyl (e.g., methyl, ethyl, or propyl) optionally substituted with one or more R^{A1}.

In some embodiments, each R^{A} independently is C₁-C₆ alkyl (e.g., methyl, ethyl, or propyl).

In some embodiments, each R^{A} independently is C₁-C₆ alkyl (e.g., methyl, ethyl, or propyl) substituted with one or more halogen or cyano.

In some embodiments, each R^{A} independently is C₁-C₆ alkyl (e.g., methyl, ethyl, or propyl) substituted with one or more R^{A1}.

In some embodiments, each R^{A} independently is C₂-C₆ alkenyl optionally substituted with one or more halogen or cyano.

In some embodiments, each R^{A} independently is C₂-C₆ alkenyl optionally substituted with one or more R^{A1}.

In some embodiments, each R^{A} independently is C₂-C₆ alkenyl.

In some embodiments, each R^{A} independently is C₂-C₆ alkenyl substituted with one or more halogen or cyano.

In some embodiments, each R^{A} independently is C₂-C₆ alkenyl substituted with one or more R^{A1}.

In some embodiments, each R^{A} independently is C₂-C₆ alkynyl optionally substituted with one or more halogen or cyano.

In some embodiments, each R^{A} independently is C₂-C₆ alkynyl optionally substituted with one or more R^{A1}.

In some embodiments, each R^{A} independently is C₂-C₆ alkynyl.

In some embodiments, each R^{A} independently is C₂-C₆ alkynyl substituted with one or more halogen or cyano.

In some embodiments, each R^{A} independently is C₂-C₆ alkynyl substituted with one or more R^{A1}.

In some embodiments, each R^{A} independently is C₁-C₆ alkoxyl optionally substituted with one or more halogen or cyano.

In some embodiments, each R^{A} independently is C₁-C₆ alkoxyl optionally substituted with one or more R^{A1}.

In some embodiments, each R^{A} independently is C₁-C₆ alkoxyl.

In some embodiments, each R^{A} independently is C₁-C₆ alkoxyl substituted with one or more halogen or cyano.

In some embodiments, each R^{A} independently is C₁-C₆ alkoxyl substituted with one or more R^{A1}.

In some embodiments, each R^{A} independently is C₃-C₈ cycloalkyl, 3- to 8-membered heterocycloalkyl, NH₂, NH(C₁-C₆ alkyl), or N(C₁-C₆ alkyl)₂, wherein the C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

In some embodiments, each R^{A} independently is C₃-C₈ cycloalkyl, 3- to 8-membered heterocycloalkyl, —O—(C₃-C₈ cycloalkyl), —O-(3- to 8-membered heterocycloalkyl), NH₂, NH(C₁-C₆ alkyl), or N(C₁-C₆ alkyl)₂, wherein the C₃-C₈ cycloalkyl, 3- to 8-membered heterocycloalkyl, —O—(C₃-C₈ cycloalkyl), —O-(3- to 8-membered heterocycloalkyl), NH(C₁-C₆ alkyl), or N(C₁-C₆ alkyl)₂ is optionally substituted with one or more halogen or cyano.

In some embodiments, each R^{A} independently is C₃-C₈ cycloalkyl optionally substituted with one or more halogen or cyano.

In some embodiments, each R^{A} independently is C₃-C₈ cycloalkyl optionally substituted with one or more R^{A1}.

In some embodiments, each R^{A} independently is 3- to 8-membered heterocycloalkyl optionally substituted with one or more halogen or cyano.

In some embodiments, each R^{A} independently is 3- to 8-membered heterocycloalkyl optionally substituted with one or more R^{A1}.

In some embodiments, each R^{A} independently is —O—(C₃-C₈ cycloalkyl), or —O-(3- to 8-membered heterocycloalkyl) optionally substituted with one or more R^{A1}.

In some embodiments, each R^{A} independently is —O—(C₃-C₈ cycloalkyl) optionally substituted with one or more R^{A1}.

In some embodiments, each R^{A} independently is —O-(3- to 8-membered heterocycloalkyl) optionally substituted with one or more R^{A1}.

In some embodiments, each R^{A} independently is NH₂, NH(C₁-C₆ alkyl), or N(C₁-C₆ alkyl)₂, wherein the NH(C₁-C₆ alkyl) or N(C₁-C₆ alkyl)₂ is optionally substituted with one or more halogen or cyano.

In some embodiments, each R^{A} independently is NH₂, NH(C₁-C₆ alkyl), or N(C₁-C₆ alkyl)₂, wherein the NH(C₁-C₆ alkyl) or N(C₁-C₆ alkyl)₂ is optionally substituted with one or more R^{A1}.

In some embodiments, each R^{A} independently is NH₂.

In some embodiments, each R^{A} independently is NH(C₁-C₆ alkyl) optionally substituted with one or more halogen or cyano.

In some embodiments, each R^{A} independently is NH(C₁-C₆ alkyl) optionally substituted with one or more R^{A1}.

In some embodiments, each R^{A} independently is N(C₁-C₆ alkyl)₂ optionally substituted with one or more halogen or cyano.

In some embodiments, each R^{A} independently is N(C₁-C₆ alkyl)₂ optionally substituted with one or more R^{A1}.

In some embodiments, each R^{A1} independently is halogen, cyano, OH, NH₂, NH(C₁-C₆ alkyl), N(C₁-C₆ alkyl)₂, C₁-C₆ alkyl, C₂-C₆ alkenyl, or C₂-C₆ alkynyl.

In some embodiments, each R^{A1} independently is halogen, cyano, OH, NH₂, NH(C₁-C₆ alkyl), N(C₁-C₆ alkyl)₂, C₁-C₆ alkyl, C₂-C₆ alkenyl, or C₂-C₆ alkynyl.

In some embodiments, each R^{A1} independently is halogen.

In some embodiments, each R^{A1} independently is F, Cl, or Br.

In some embodiments, each R^{A1} independently is F.

In some embodiments, each R^{A1} independently is C₁.

In some embodiments, each R^{A1} independently is Br.

In some embodiments, each R^{A1} independently is cyano.

In some embodiments, each R^{A1} independently is OH.

In some embodiments, each R^{A1} independently is NH₂, NH(C₁-C₆ alkyl), or N(C₁-C₆ alkyl)₂.

In some embodiments, each R^{A1} independently is NH₂.

In some embodiments, each R^{A1} independently is NH(C₁-C₆ alkyl), or N(C₁-C₆ alkyl)₂.

In some embodiments, each R^{A1} independently is NH(C₁-C₆ alkyl).

In some embodiments, each R^{A1} independently is N(C₁-C₆ alkyl)₂.

In some embodiments, each R^{A1} independently is C₁-C₆ alkyl, C₂-C₆ alkenyl, or C₂-C₆ alkynyl.

In some embodiments, each R^{A1} independently is C₁-C₆ alkyl (e.g., methyl, ethyl, or propyl).

In some embodiments, each R^{A1} independently is C₂-C₆ alkenyl, or C₂-C₆ alkynyl.

### Embodiments of the Compounds

In some embodiments, when W¹, W², W³, W⁵, and W⁶ each are CH; X¹ is NH; X² is —NHC(O)—* or —C(O)NH—*; and R¹ is F, Cl, or CH₃;
then A is not

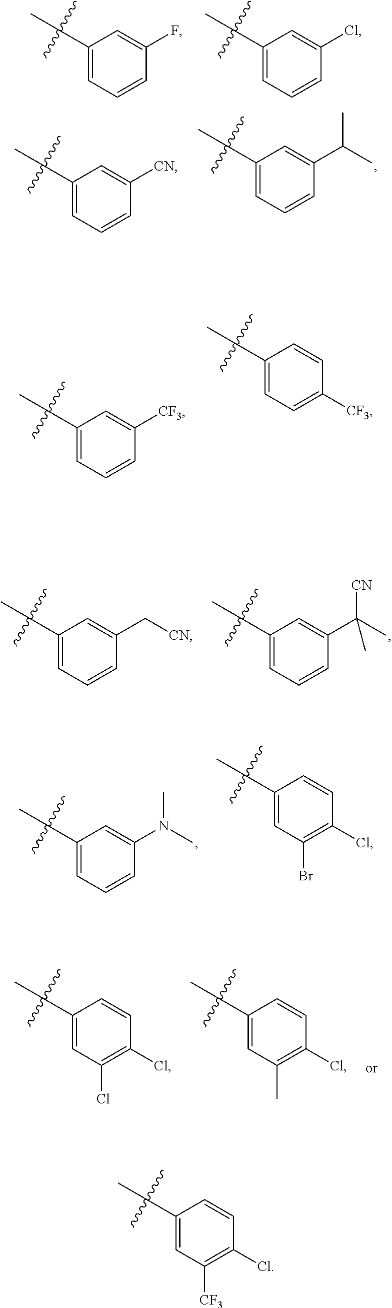

In some embodiments, when W¹, W², W³, W⁵, and W⁶ each are CH; X¹ is NH; X² is —NHC(O)—* or —C(O)NH—*; and R¹ is F, Cl, or CH₃;
then A is not phenyl substituted with one or more F, Cl, Br, CN, CH₃, CH(CH₃)₂, CF₃; CH₂CN, C(CH₃)₂CN, or N(CH₃)₂.

In some embodiments, when W¹, W², W³, W⁵, and W⁶ each are CH; X¹ is NH; X² is —NHC(O)—* or —C(O)NH—*; and R¹ is halogen or C₁-C₆ alkyl;
then A is not C₆-C₁₀ aryl substituted with one or more halogen, cyano, C₁-C₆ alkyl optionally substituted with one or more halogen or cyano, or N(C₁-C₆ alkyl)₂.

In some embodiments, when W¹, W², W³, W⁵, and W⁶ each are CH; X¹ is NH; X² is —NHC(O)—* or —C(O)NH—*; and R¹ is halogen or C₁-C₆ alkyl;
then A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}, and each R^{A} independently is C₂-C₆ alkenyl, C₂-C₆ alkynyl, C₁-C₆ alkoxyl, C₃-C₈ cycloalkyl, or 3- to 8-membered heterocycloalkyl, wherein the C₂-C₆ alkenyl, C₂-C₆ alkynyl, C₁-C₆ alkoxyl, C₃-C₈ cycloalkyl, or 3- to 8-membered heterocycloalkyl is optionally substituted with one or more halogen or cyano.

In some embodiments, when R¹ is F, Cl, or CH₃; then A is not

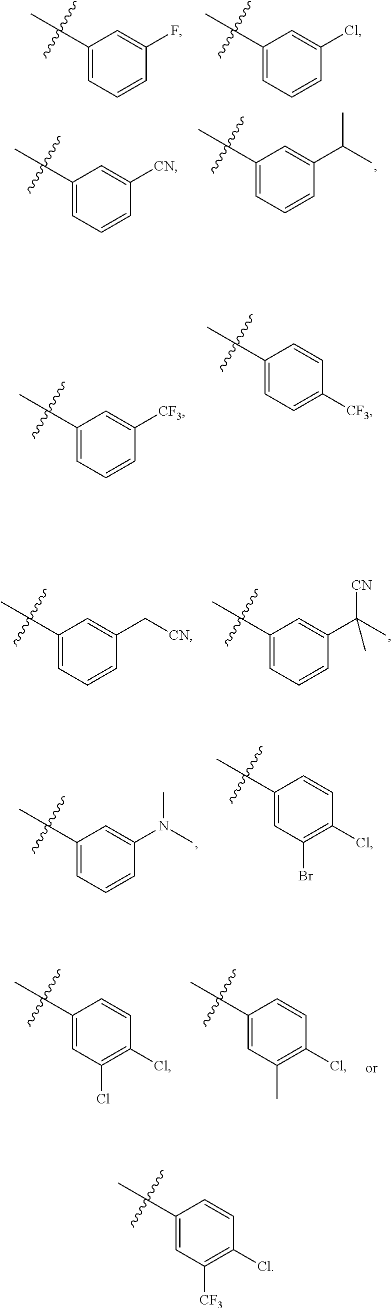

In some embodiments, when R¹ is F, Cl, or CH₃; then A is not phenyl substituted with one or more F, Cl, Br, CN, CH₃, CH(CH₃)₂, CF₃, CH₂CN, C(CH₃)₂CN, or N(CH₃)₂.

In some embodiments, when R¹ is halogen or C₁-C₆ alkyl; then A is not C₆-C₁₀ aryl substituted with one or more halogen, cyano, C₁-C₆ alkyl optionally substituted with one or more halogen or cyano, or N(C₁-C₆ alkyl)₂.

In some embodiments, when R¹ is halogen or C₁-C₆ alkyl; then A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}; and each R^{A} independently is C₂-C₆ alkenyl, C₂-C₆ alkynyl, C₁-C₆ alkoxyl, C₃-C₈ cycloalkyl, or 3- to 8-membered heterocycloalkyl, wherein the C₂-C₆ alkenyl, C₂-C₆ alkynyl, C₁-C₆ alkoxyl, C₃-C₈ cycloalkyl, or 3- to 8-membered heterocycloalkyl is optionally substituted with one or more halogen or cyano.

In some embodiments, the compound is of Formula (II):

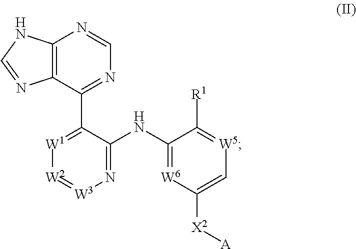

or a pharmaceutically acceptable salt or stereoisomer thereof.

In some embodiments, the compound is of Formula (III-a), (III-b), (III-c), (III-d), (III-e), or (III-f):

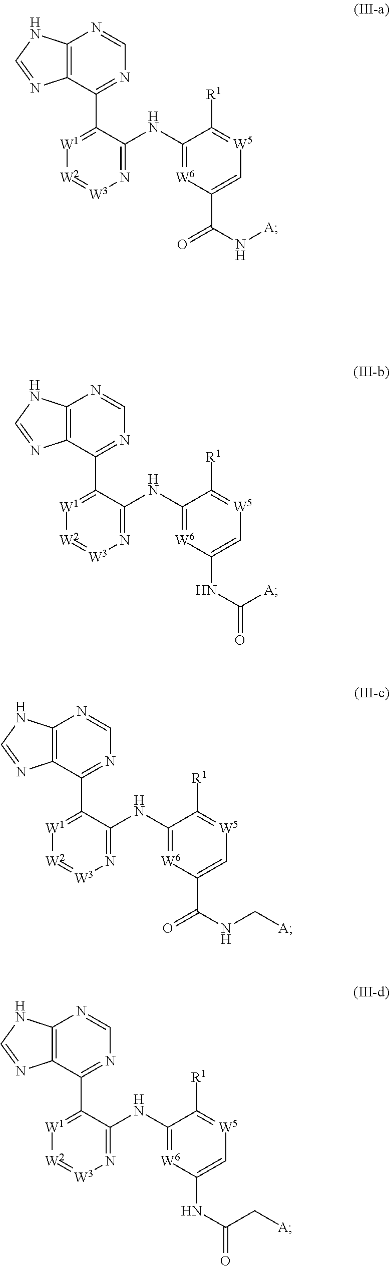

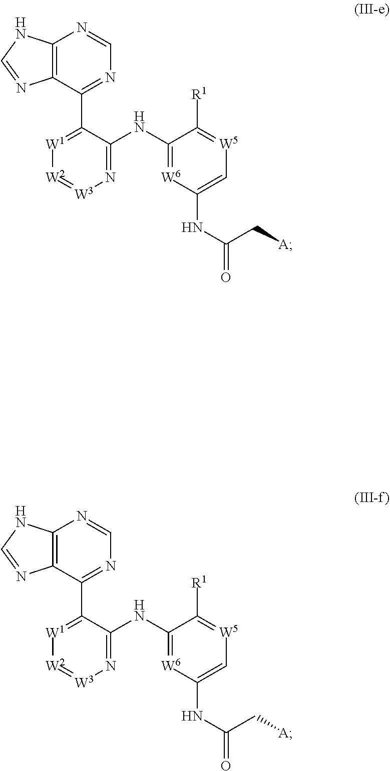

or a pharmaceutically acceptable salt or stereoisomer thereof.

In some embodiments, the compound is of Formula (IV-a) or (IV-b):

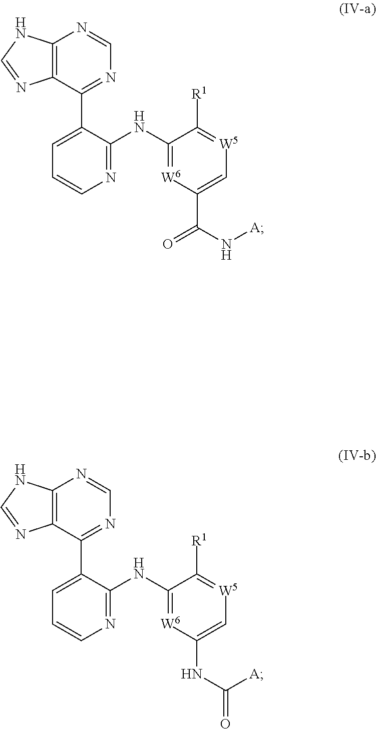

or a pharmaceutically acceptable salt or stereoisomer thereof.

In some embodiments, the compound is a compound described in Table I, IA, or II, or a pharmaceutically acceptable salt or stereoisomer thereof.

In some embodiments, the compound is a compound described in Table I or IA, or a pharmaceutically acceptable salt or stereoisomer thereof.

In some embodiments, the compound is a compound described in Table I, or a pharmaceutically acceptable salt or stereoisomer thereof.

In some embodiments, the compound is a compound described in Table IA, or a pharmaceutically acceptable salt or stereoisomer thereof.

In some embodiments, the compound is a compound described in Table II, or a pharmaceutically acceptable salt or stereoisomer thereof.

In some embodiments, the compound is a compound described in Table I, IA or II, or a pharmaceutically acceptable salt thereof.

In some embodiments, the compound is a compound described in Table I or IA, or a pharmaceutically acceptable salt thereof.

In some embodiments, the compound is a compound described in Table I, or a pharmaceutically acceptable salt thereof.

In some embodiments, the compound is a compound described in Table IA, or a pharmaceutically acceptable salt thereof.

In some embodiments, the compound is a compound described in Table II, or a pharmaceutically acceptable salt thereof.

In some embodiments, the compound is selected from the compounds described in Table I, IA or II.

In some embodiments, the compound is selected from the compounds described in Table I or IA.

In some embodiments, the compound is selected from the compounds described in Table I.

In some embodiments, the compound is selected from the compounds described in Table IA.

In some embodiments, the compound is selected from the compounds described in Table II.

TABLE I

| Compound No. | Structure |
|---|---|
| I-1 | 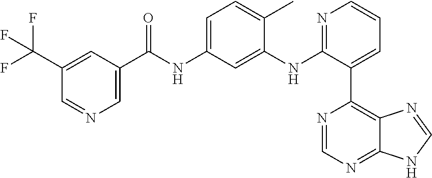 [SMILES: Cc1ccc(NC(=O)c2cncc(C(F)(F)F)c2)cc1Nc1ncccc1-c1ncnc2[nH]cnc12] |
| I-2 | 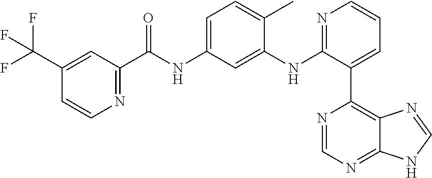 [SMILES: Cc1ccc(NC(=O)c2cc(C(F)(F)F)ccn2)cc1Nc1ncccc1-c1ncnc2[nH]cnc12] |
| I-3 | 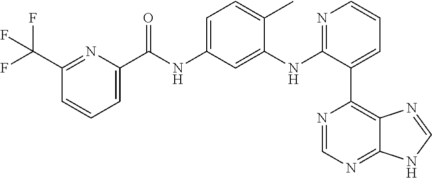 [SMILES: Cc1ccc(NC(=O)c2cccc(C(F)(F)F)n2)cc1Nc1ncccc1-c1ncnc2[nH]cnc12] |
| I-4 | 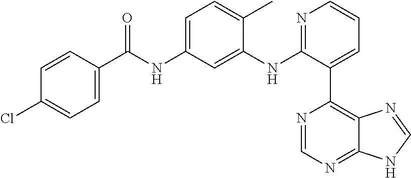 [SMILES: Cc1ccc(NC(=O)c2ccc(Cl)cc2)cc1Nc1ncccc1-c1ncnc2[nH]cnc12] |
| I-5 | 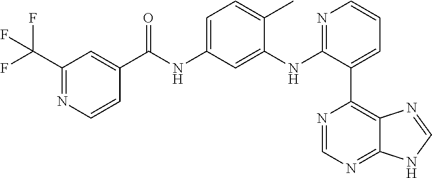 [SMILES: Cc1ccc(NC(=O)c2ccnc(C(F)(F)F)c2)cc1Nc1ncccc1-c1ncnc2[nH]cnc12] |
| I-6 | 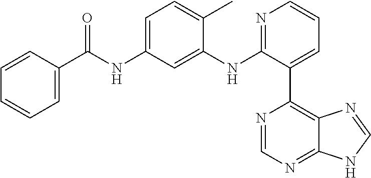 [SMILES: Cc1ccc(NC(=O)c2ccccc2)cc1Nc1ncccc1-c1ncnc2[nH]cnc12] |
| I-7 | 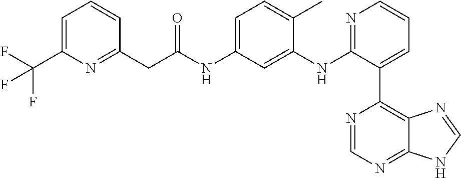 [SMILES: Cc1ccc(NC(=O)Cc2cccc(C(F)(F)F)n2)cc1Nc1ncccc1-c1ncnc2[nH]cnc12] |
| I-8 | 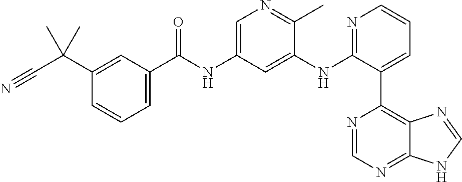 [SMILES: Cc1ncc(NC(=O)c2cccc(C(C)(C)C#N)c2)cc1Nc1ncccc1-c1ncnc2[nH]cnc12] |
| I-9 | 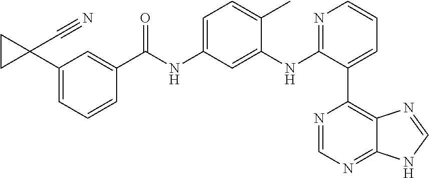 [SMILES: Cc1ccc(NC(=O)c2cccc(C3(C#N)CC3)c2)cc1Nc1ncccc1-c1ncnc2[nH]cnc12] |
| I-10 | 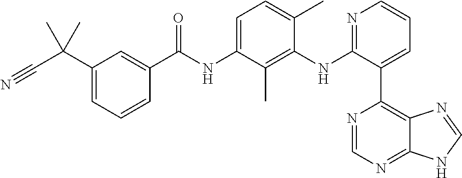 [SMILES: Cc1ccc(NC(=O)c2cccc(C(C)(C)C#N)c2)c(C)c1Nc1ncccc1-c1ncnc2[nH]cnc12] |
| I-11 | 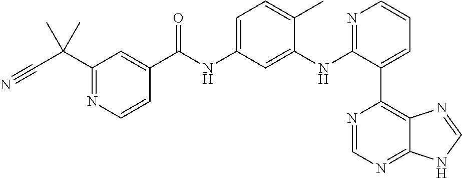 [SMILES: Cc1ccc(NC(=O)c2ccnc(C(C)(C)C#N)c2)cc1Nc1ncccc1-c1ncnc2[nH]cnc12] |
| I-12 | 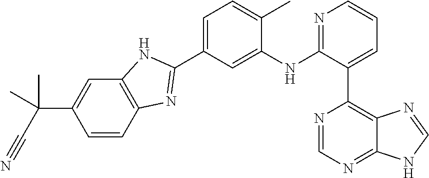 [SMILES: Cc1ccc(-c2nc3ccc(C(C)(C)C#N)cc3[nH]2)cc1Nc1ncccc1-c1ncnc2[nH]cnc12] |
| I-13 | 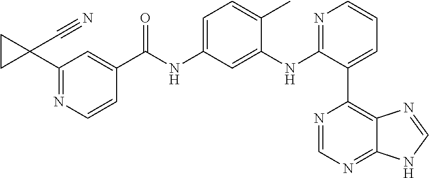 [SMILES: Cc1ccc(NC(=O)c2ccnc(C3(C#N)CC3)c2)cc1Nc1ncccc1-c1ncnc2[nH]cnc12] |
| I-14 | 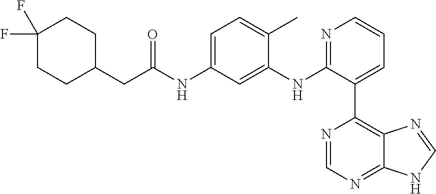 [SMILES: Cc1ccc(NC(=O)CC2CCC(F)(F)CC2)cc1Nc1ncccc1-c1ncnc2[nH]cnc12] |
| I-15 | 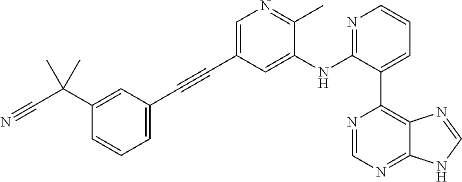 [SMILES: Cc1ncc(C#Cc2cccc(C(C)(C)C#N)c2)cc1Nc1ncccc1-c1ncnc2[nH]cnc12] |
| I-16 | 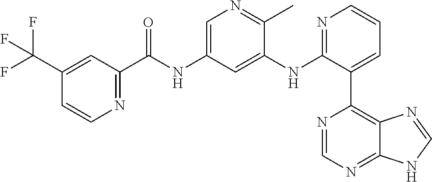 [SMILES: Cc1ncc(NC(=O)c2cc(C(F)(F)F)ccn2)cc1Nc1ncccc1-c1ncnc2[nH]cnc12] |
| I-17 | 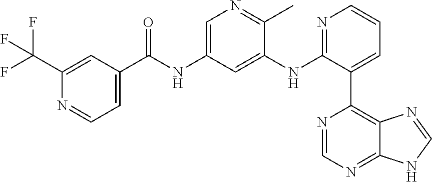 [SMILES: Cc1ncc(NC(=O)c2ccnc(C(F)(F)F)c2)cc1Nc1ncccc1-c1ncnc2[nH]cnc12] |
| I-18 | 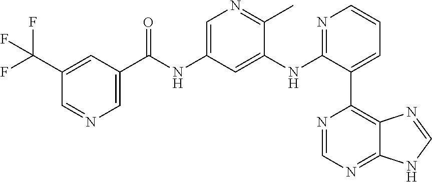 [SMILES: Cc1ncc(NC(=O)c2cncc(C(F)(F)F)c2)cc1Nc1ncccc1-c1ncnc2[nH]cnc12] |
| I-19 | 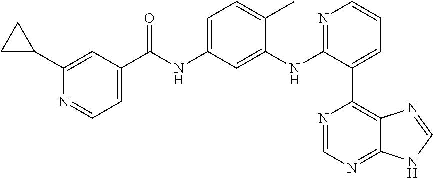 [SMILES: Cc1ccc(NC(=O)c2ccnc(C3CC3)c2)cc1Nc1ncccc1-c1ncnc2[nH]cnc12] |
| I-20 | 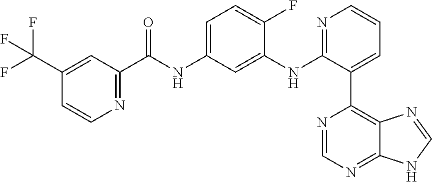 [SMILES: O=C(Nc1ccc(F)c(Nc2ncccc2-c2ncnc3[nH]cnc23)c1)c1cc(C(F)(F)F)ccn1] |
| I-21 | 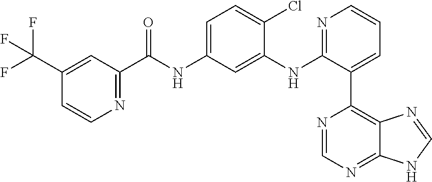 [SMILES: O=C(Nc1ccc(Cl)c(Nc2ncccc2-c2ncnc3[nH]cnc23)c1)c1cc(C(F)(F)F)ccn1] |
| I-22 | 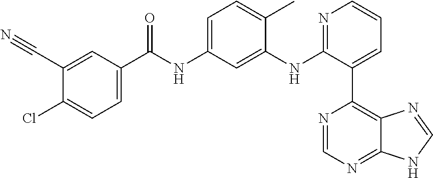 [SMILES: Cc1ccc(NC(=O)c2ccc(Cl)c(C#N)c2)cc1Nc1ncccc1-c1ncnc2[nH]cnc12] |
| I-23 | 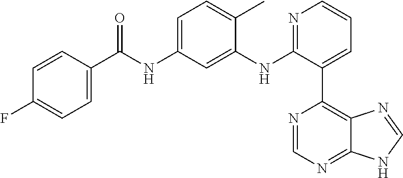 [SMILES: Cc1ccc(NC(=O)c2ccc(F)cc2)cc1Nc1ncccc1-c1ncnc2[nH]cnc12] |
| I-24 | 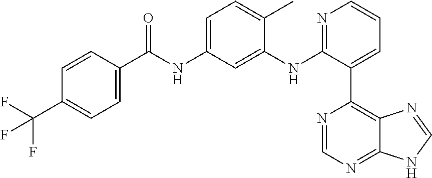 [SMILES: Cc1ccc(NC(=O)c2ccc(C(F)(F)F)cc2)cc1Nc1ncccc1-c1ncnc2[nH]cnc12] |
| I-25 | 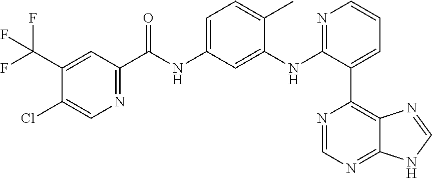 [SMILES: Cc1ccc(NC(=O)c2cc(C(F)(F)F)c(Cl)cn2)cc1Nc1ncccc1-c1ncnc2[nH]cnc12] |
| I-26 | 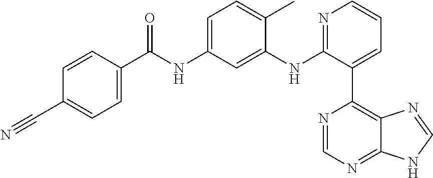 [SMILES: Cc1ccc(NC(=O)c2ccc(C#N)cc2)cc1Nc1ncccc1-c1ncnc2[nH]cnc12] |
| I-27 | 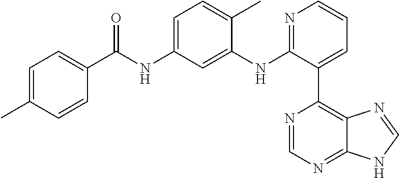 [SMILES: Cc1ccc(C(=O)Nc2ccc(C)c(Nc3ncccc3-c3ncnc4[nH]cnc34)c2)cc1] |
| I-28 | 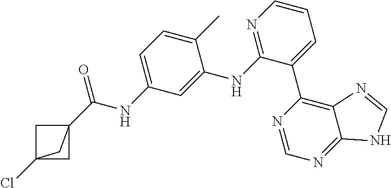 [SMILES: Cc1ccc(NC(=O)C23CC(Cl)(C2)C3)cc1Nc1ncccc1-c1ncnc2[nH]cnc12] |
| I-29 | 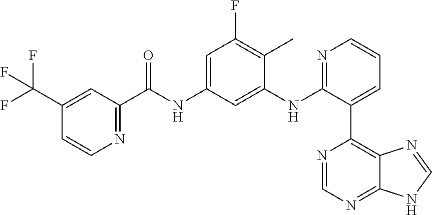 [SMILES: Cc1c(F)cc(NC(=O)c2cc(C(F)(F)F)ccn2)cc1Nc1ncccc1-c1ncnc2[nH]cnc12] |
| I-30 | 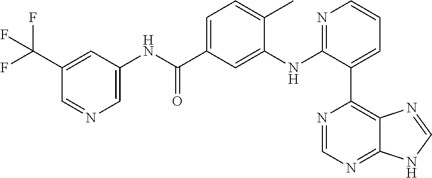 [SMILES: Cc1ccc(C(=O)Nc2cncc(C(F)(F)F)c2)cc1Nc1ncccc1-c1ncnc2[nH]cnc12] |
| I-31 | 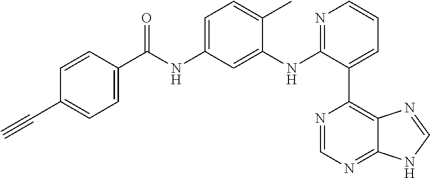 [SMILES: C#Cc1ccc(C(=O)Nc2ccc(C)c(Nc3ncccc3-c3ncnc4[nH]cnc34)c2)cc1] |
| I-32 | 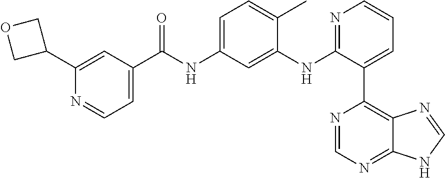 [SMILES: Cc1ccc(NC(=O)c2ccnc(C3COC3)c2)cc1Nc1ncccc1-c1ncnc2[nH]cnc12] |
| I-33 | 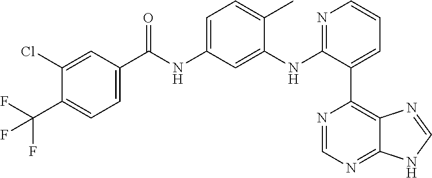 [SMILES: Cc1ccc(NC(=O)c2ccc(C(F)(F)F)c(Cl)c2)cc1Nc1ncccc1-c1ncnc2[nH]cnc12] |
| I-34 | 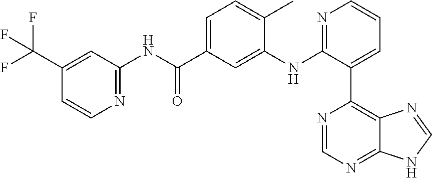 [SMILES: Cc1ccc(C(=O)Nc2cc(C(F)(F)F)ccn2)cc1Nc1ncccc1-c1ncnc2[nH]cnc12] |
| I-35 | 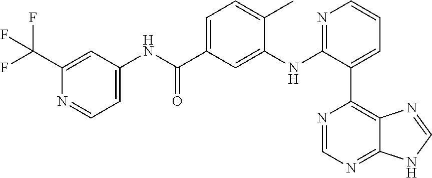 [SMILES: Cc1ccc(C(=O)Nc2ccnc(C(F)(F)F)c2)cc1Nc1ncccc1-c1ncnc2[nH]cnc12] |
| I-36 | 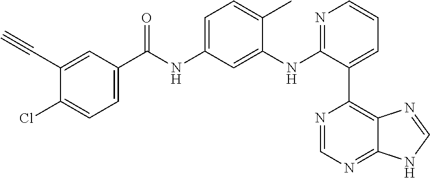 [SMILES: C#Cc1cc(C(=O)Nc2ccc(C)c(Nc3ncccc3-c3ncnc4[nH]cnc34)c2)ccc1Cl] |
| I-37 | 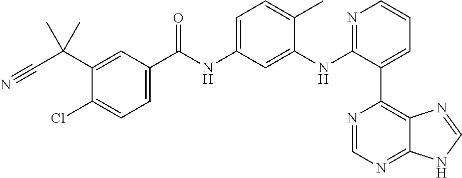 [SMILES: Cc1ccc(NC(=O)c2ccc(Cl)c(C(C)(C)C#N)c2)cc1Nc1ncccc1-c1ncnc2[nH]cnc12] |
| I-38 | 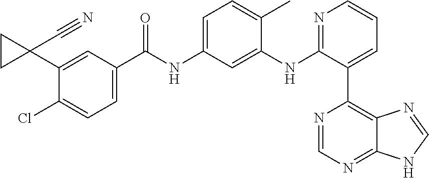 [SMILES: Cc1ccc(NC(=O)c2ccc(Cl)c(C3(C#N)CC3)c2)cc1Nc1ncccc1-c1ncnc2[nH]cnc12] |
| I-39 | 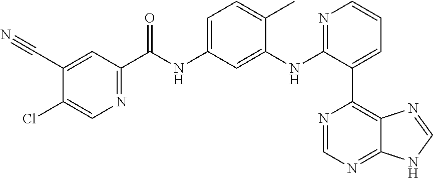 [SMILES: Cc1ccc(NC(=O)c2cc(C#N)c(Cl)cn2)cc1Nc1ncccc1-c1ncnc2[nH]cnc12] |
| I-40 | 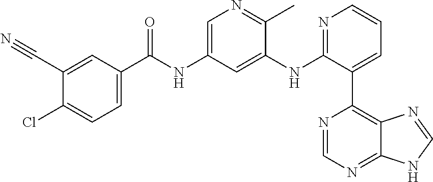 [SMILES: Cc1ncc(NC(=O)c2ccc(Cl)c(C#N)c2)cc1Nc1ncccc1-c1ncnc2[nH]cnc12] |
| I-41 | 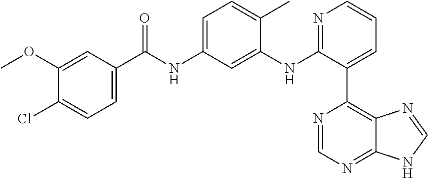 [SMILES: COc1cc(C(=O)Nc2ccc(C)c(Nc3ncccc3-c3ncnc4[nH]cnc34)c2)ccc1Cl] |
| I-42 | 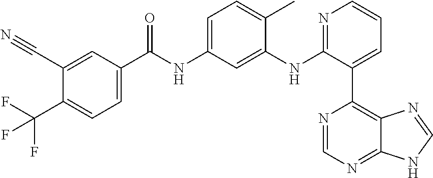 [SMILES: Cc1ccc(NC(=O)c2ccc(C(F)(F)F)c(C#N)c2)cc1Nc1ncccc1-c1ncnc2[nH]cnc12] |
| I-43 | 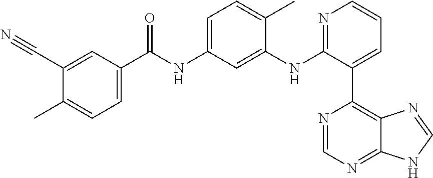 [SMILES: Cc1ccc(C(=O)Nc2ccc(C)c(Nc3ncccc3-c3ncnc4[nH]cnc34)c2)cc1C#N] |
| I-44 | 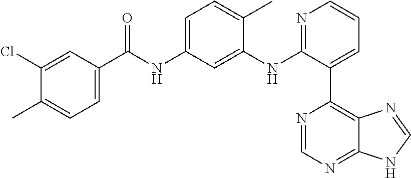 [SMILES: Cc1ccc(C(=O)Nc2ccc(C)c(Nc3ncccc3-c3ncnc4[nH]cnc34)c2)cc1Cl] |
| I-45 | 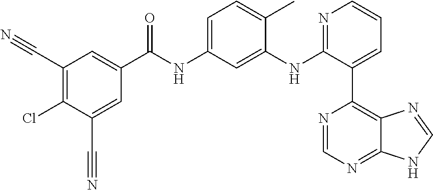 [SMILES: Cc1ccc(NC(=O)c2cc(C#N)c(Cl)c(C#N)c2)cc1Nc1ncccc1-c1ncnc2[nH]cnc12] |
| I-46 | 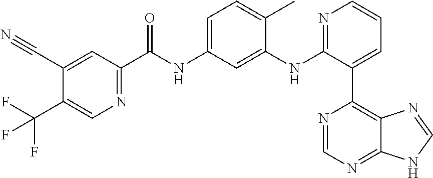 [SMILES: Cc1ccc(NC(=O)c2cc(C#N)c(C(F)(F)F)cn2)cc1Nc1ncccc1-c1ncnc2[nH]cnc12] |
| I-47 | 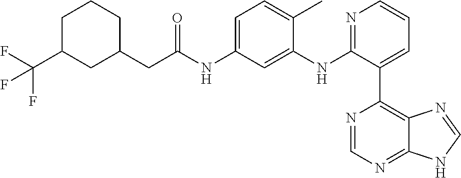 [SMILES: Cc1ccc(NC(=O)CC2CCCC(C(F)(F)F)C2)cc1Nc1ncccc1-c1ncnc2[nH]cnc12] |
| I-48 | 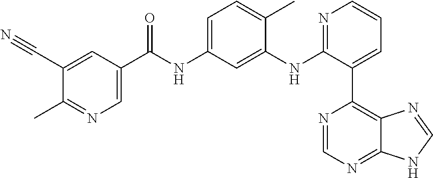 [SMILES: Cc1ccc(NC(=O)c2cnc(C)c(C#N)c2)cc1Nc1ncccc1-c1ncnc2[nH]cnc12] |
| I-49 | 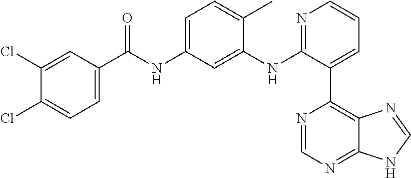 [SMILES: Cc1ccc(NC(=O)c2ccc(Cl)c(Cl)c2)cc1Nc1ncccc1-c1ncnc2[nH]cnc12] |
| I-50 | 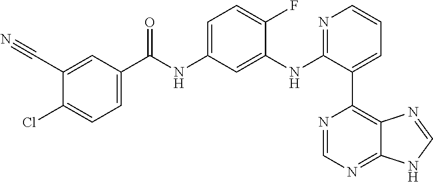 [SMILES: N#Cc1cc(C(=O)Nc2ccc(F)c(Nc3ncccc3-c3ncnc4[nH]cnc34)c2)ccc1Cl] |
| I-51 | 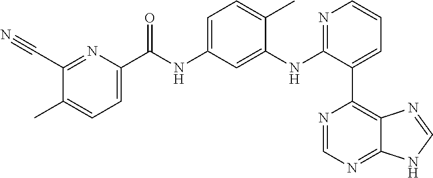 [SMILES: Cc1ccc(NC(=O)c2ccc(C)c(C#N)n2)cc1Nc1ncccc1-c1ncnc2[nH]cnc12] |
| I-52 | 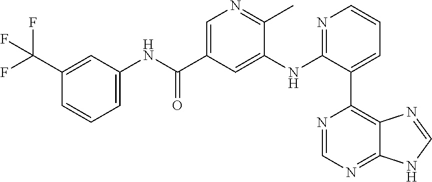 [SMILES: Cc1ncc(C(=O)Nc2cccc(C(F)(F)F)c2)cc1Nc1ncccc1-c1ncnc2[nH]cnc12] |
| I-53 | 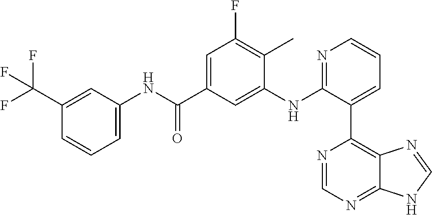 [SMILES: Cc1c(F)cc(C(=O)Nc2cccc(C(F)(F)F)c2)cc1Nc1ncccc1-c1ncnc2[nH]cnc12] |
| I-54 | 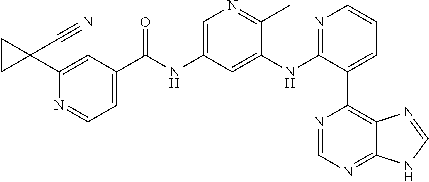 [SMILES: Cc1ncc(NC(=O)c2ccnc(C3(C#N)CC3)c2)cc1Nc1ncccc1-c1ncnc2[nH]cnc12] |
| I-55 | 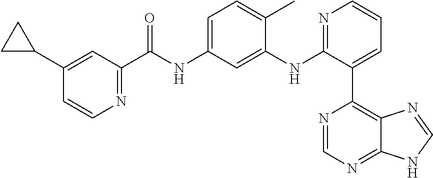 [SMILES: Cc1ccc(NC(=O)c2cc(C3CC3)ccn2)cc1Nc1ncccc1-c1ncnc2[nH]cnc12] |
| I-56 | 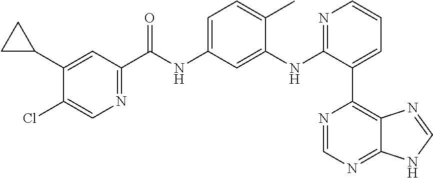 [SMILES: Cc1ccc(NC(=O)c2cc(C3CC3)c(Cl)cn2)cc1Nc1ncccc1-c1ncnc2[nH]cnc12] |
| I-57 | 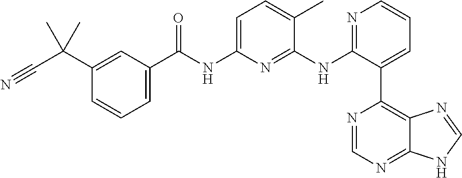 [SMILES: Cc1ccc(NC(=O)c2cccc(C(C)(C)C#N)c2)nc1Nc1ncccc1-c1ncnc2[nH]cnc12] |
| I-58 | 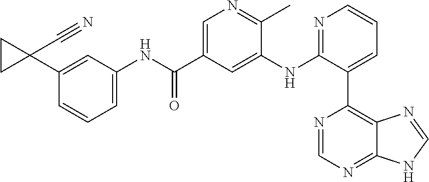 [SMILES: Cc1ncc(C(=O)Nc2cccc(C3(C#N)CC3)c2)cc1Nc1ncccc1-c1ncnc2[nH]cnc12] |
| I-59 | 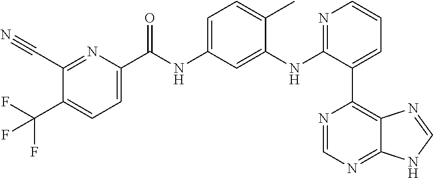 [SMILES: Cc1ccc(NC(=O)c2ccc(C(F)(F)F)c(C#N)n2)cc1Nc1ncccc1-c1ncnc2[nH]cnc12] |
| I-60 | 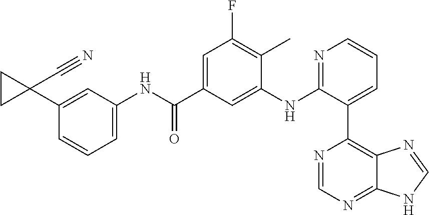 [SMILES: Cc1c(F)cc(C(=O)Nc2cccc(C3(C#N)CC3)c2)cc1Nc1ncccc1-c1ncnc2[nH]cnc12] |
| I-61 | 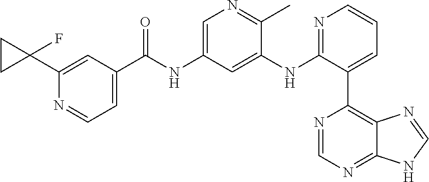 [SMILES: Cc1ncc(NC(=O)c2ccnc(C3(F)CC3)c2)cc1Nc1ncccc1-c1ncnc2[nH]cnc12] |
| I-62 | 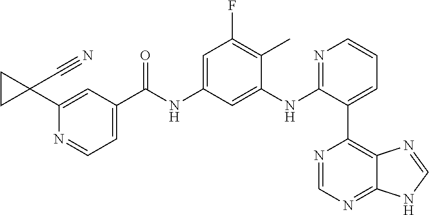 [SMILES: Cc1c(F)cc(NC(=O)c2ccnc(C3(C#N)CC3)c2)cc1Nc1ncccc1-c1ncnc2[nH]cnc12] |
| I-63 | 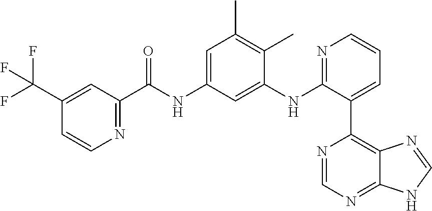 [SMILES: Cc1cc(NC(=O)c2cc(C(F)(F)F)ccn2)cc(Nc2ncccc2-c2ncnc3[nH]cnc23)c1C] |
| I-64 | 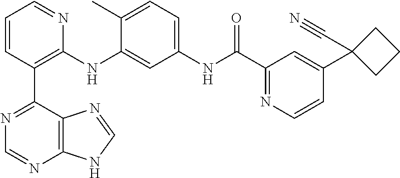 [SMILES: Cc1ccc(NC(=O)c2cc(C3(C#N)CCC3)ccn2)cc1Nc1ncccc1-c1ncnc2[nH]cnc12] |
| I-65 | 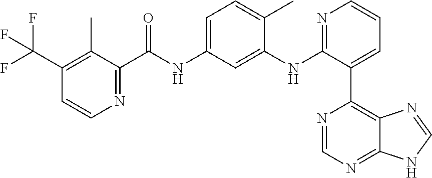 [SMILES: Cc1ccc(NC(=O)c2nccc(C(F)(F)F)c2C)cc1Nc1ncccc1-c1ncnc2[nH]cnc12] |
| I-66 | 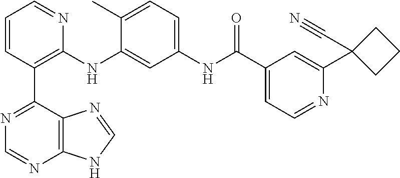 [SMILES: Cc1ccc(NC(=O)c2ccnc(C3(C#N)CCC3)c2)cc1Nc1ncccc1-c1ncnc2[nH]cnc12] |
| I-67 | 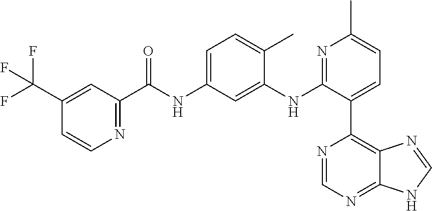 [SMILES: Cc1ccc(-c2ncnc3[nH]cnc23)c(Nc2cc(NC(=O)c3cc(C(F)(F)F)ccn3)ccc2C)n1] |
| I-68 | 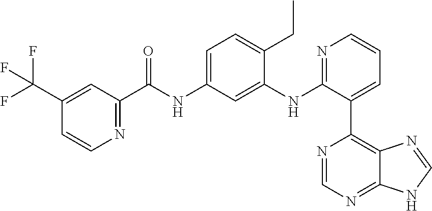 [SMILES: CCc1ccc(NC(=O)c2cc(C(F)(F)F)ccn2)cc1Nc1ncccc1-c1ncnc2[nH]cnc12] |
| I-69 | 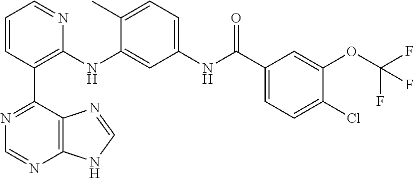 [SMILES: Cc1ccc(NC(=O)c2ccc(Cl)c(OC(F)(F)F)c2)cc1Nc1ncccc1-c1ncnc2[nH]cnc12] |
| I-70 | 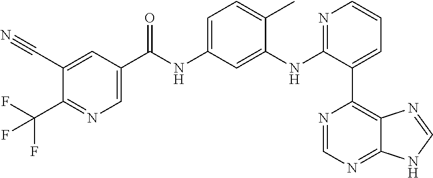 [SMILES: Cc1ccc(NC(=O)c2cnc(C(F)(F)F)c(C#N)c2)cc1Nc1ncccc1-c1ncnc2[nH]cnc12] |
| I-71 | 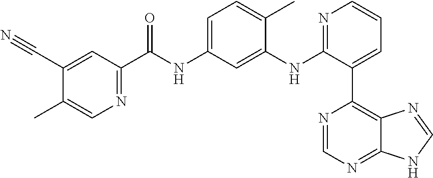 [SMILES: Cc1cnc(C(=O)Nc2ccc(C)c(Nc3ncccc3-c3ncnc4[nH]cnc34)c2)cc1C#N] |
| I-72 | 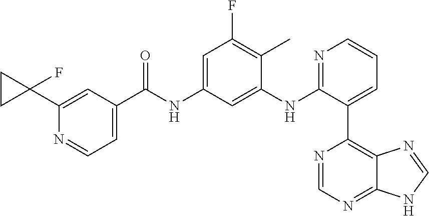 [SMILES: Cc1c(F)cc(NC(=O)c2ccnc(C3(F)CC3)c2)cc1Nc1ncccc1-c1ncnc2[nH]cnc12] |
| I-73 | 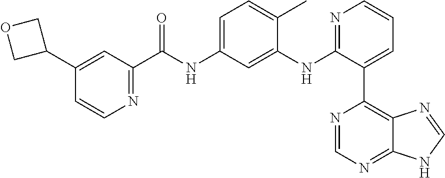 [SMILES: Cc1ccc(NC(=O)c2cc(C3COC3)ccn2)cc1Nc1ncccc1-c1ncnc2[nH]cnc12] |
| I-74 | 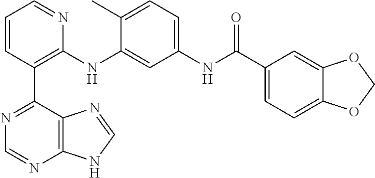 [SMILES: Cc1ccc(NC(=O)c2ccc3c(c2)OCO3)cc1Nc1ncccc1-c1ncnc2[nH]cnc12] |
| I-75 | 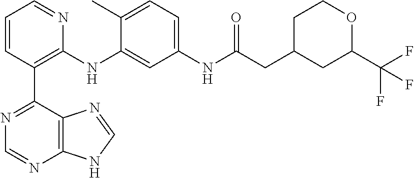 [SMILES: Cc1ccc(NC(=O)CC2CCOC(C(F)(F)F)C2)cc1Nc1ncccc1-c1ncnc2[nH]cnc12] |
| I-76 | 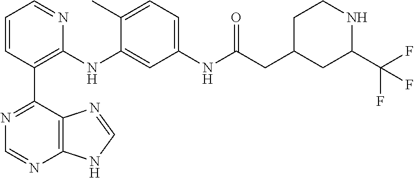 [SMILES: Cc1ccc(NC(=O)CC2CCNC(C(F)(F)F)C2)cc1Nc1ncccc1-c1ncnc2[nH]cnc12] |
| I-77 | 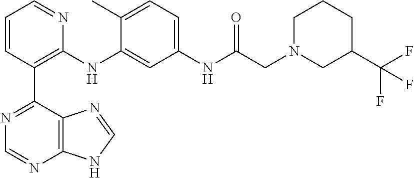 [SMILES: Cc1ccc(NC(=O)CN2CCCC(C(F)(F)F)C2)cc1Nc1ncccc1-c1ncnc2[nH]cnc12] |
| I-78 | 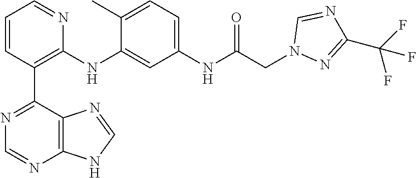 [SMILES: Cc1ccc(NC(=O)Cn2cnc(C(F)(F)F)n2)cc1Nc1ncccc1-c1ncnc2[nH]cnc12] |
| I-79 | 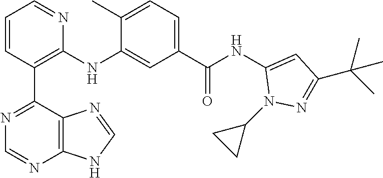 [SMILES: Cc1ccc(C(=O)Nc2cc(C(C)(C)C)nn2C2CC2)cc1Nc1ncccc1-c1ncnc2[nH]cnc12] |
| I-80 | 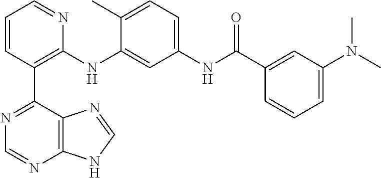 [SMILES: Cc1ccc(NC(=O)c2cccc(N(C)C)c2)cc1Nc1ncccc1-c1ncnc2[nH]cnc12] |
| I-81 | 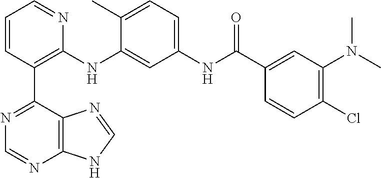 [SMILES: Cc1ccc(NC(=O)c2ccc(Cl)c(N(C)C)c2)cc1Nc1ncccc1-c1ncnc2[nH]cnc12] |
| I-82 | 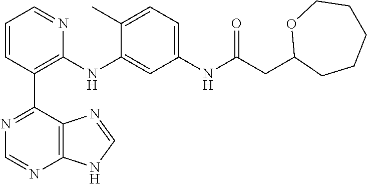 [SMILES: Cc1ccc(NC(=O)CC2CCCCCO2)cc1Nc1ncccc1-c1ncnc2[nH]cnc12] |
| I-83 | 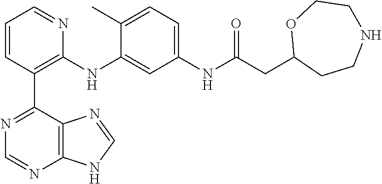 [SMILES: Cc1ccc(NC(=O)CC2CCNCCO2)cc1Nc1ncccc1-c1ncnc2[nH]cnc12] |
| I-84 | 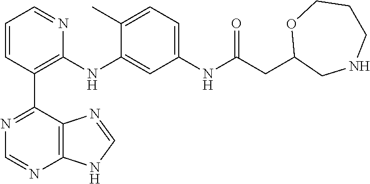 [SMILES: Cc1ccc(NC(=O)CC2CNCCCO2)cc1Nc1ncccc1-c1ncnc2[nH]cnc12] |
| I-85 | 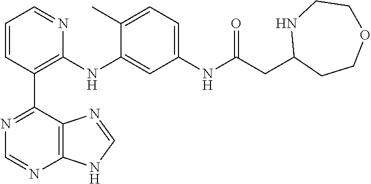 [SMILES: Cc1ccc(NC(=O)CC2CCOCCN2)cc1Nc1ncccc1-c1ncnc2[nH]cnc12] |
| I-86 | 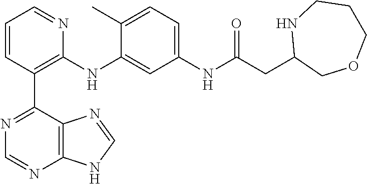 [SMILES: Cc1ccc(NC(=O)CC2COCCCN2)cc1Nc1ncccc1-c1ncnc2[nH]cnc12] |
| I-87 | 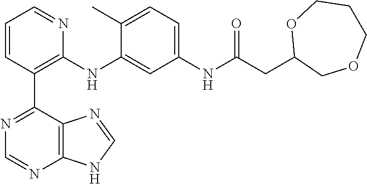 [SMILES: Cc1ccc(NC(=O)CC2COCCCO2)cc1Nc1ncccc1-c1ncnc2[nH]cnc12] |
| I-88 | 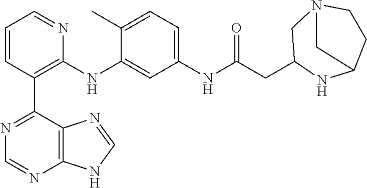 [SMILES: Cc1ccc(NC(=O)CC2CN3CCC(C3)N2)cc1Nc1ncccc1-c1ncnc2[nH]cnc12] |
| I-89 | 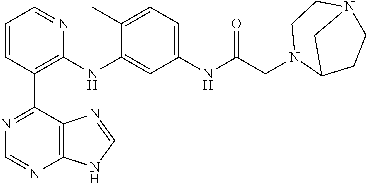 [SMILES: Cc1ccc(NC(=O)CN2CCN3CCC2C3)cc1Nc1ncccc1-c1ncnc2[nH]cnc12] |

TABLE IA

| Compound No. | Structure |
|---|---|
| IA-90 | 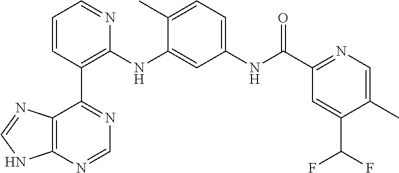 [SMILES: Cc1ccc(NC(=O)c2cc(C(F)F)c(C)cn2)cc1Nc1ncccc1-c1ncnc2[nH]cnc12] |
| IA-91 | 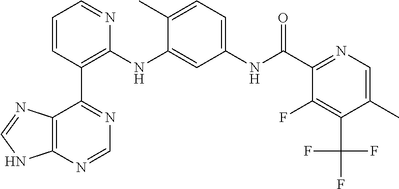 [SMILES: Cc1ccc(NC(=O)c2ncc(C)c(C(F)(F)F)c2F)cc1Nc1ncccc1-c1ncnc2[nH]cnc12] |
| IA-92 | 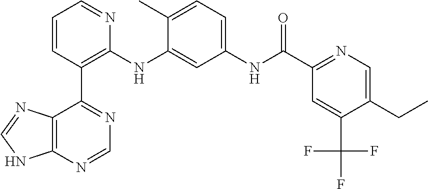 [SMILES: CCc1cnc(C(=O)Nc2ccc(C)c(Nc3ncccc3-c3ncnc4[nH]cnc34)c2)cc1C(F)(F)F] |
| IA-93 | 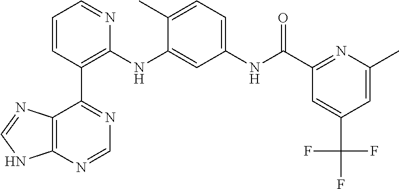 [SMILES: Cc1cc(C(F)(F)F)cc(C(=O)Nc2ccc(C)c(Nc3ncccc3-c3ncnc4[nH]cnc34)c2)n1] |
| IA-94 | 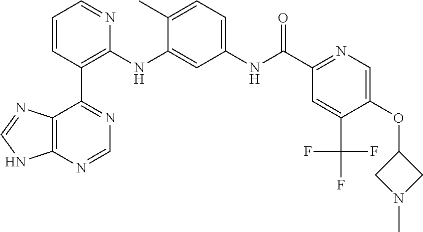 [SMILES: Cc1ccc(NC(=O)c2cc(C(F)(F)F)c(OC3CN(C)C3)cn2)cc1Nc1ncccc1-c1ncnc2[nH]cnc12] |
| IA-95 | 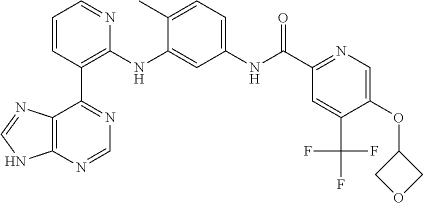 [SMILES: Cc1ccc(NC(=O)c2cc(C(F)(F)F)c(OC3COC3)cn2)cc1Nc1ncccc1-c1ncnc2[nH]cnc12] |
| IA-96 | 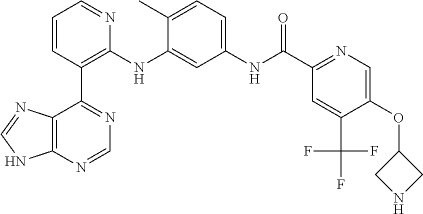 [SMILES: Cc1ccc(NC(=O)c2cc(C(F)(F)F)c(OC3CNC3)cn2)cc1Nc1ncccc1-c1ncnc2[nH]cnc12] |
| IA-97 | 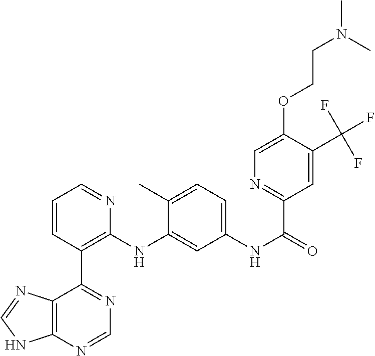 [SMILES: Cc1ccc(NC(=O)c2cc(C(F)(F)F)c(OCCN(C)C)cn2)cc1Nc1ncccc1-c1ncnc2[nH]cnc12] |
| IA-98 | 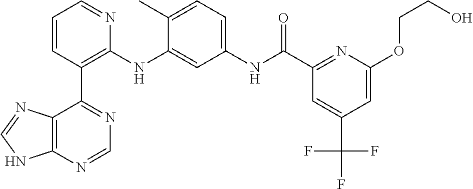 [SMILES: Cc1ccc(NC(=O)c2cc(C(F)(F)F)cc(OCCO)n2)cc1Nc1ncccc1-c1ncnc2[nH]cnc12] |
| IA-99 | 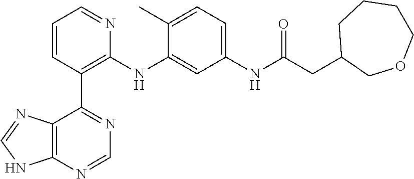 [SMILES: Cc1ccc(NC(=O)CC2CCCCOC2)cc1Nc1ncccc1-c1ncnc2[nH]cnc12] |
| IA-100 | 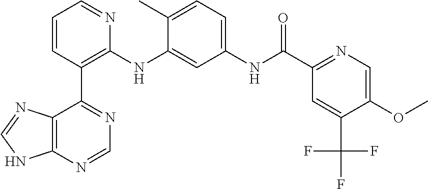 [SMILES: COc1cnc(C(=O)Nc2ccc(C)c(Nc3ncccc3-c3ncnc4[nH]cnc34)c2)cc1C(F)(F)F] |
| IA-101 | 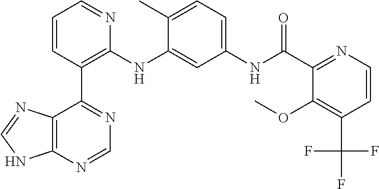 [SMILES: COc1c(C(F)(F)F)ccnc1C(=O)Nc1ccc(C)c(Nc2ncccc2-c2ncnc3[nH]cnc23)c1] |
| IA-102 | 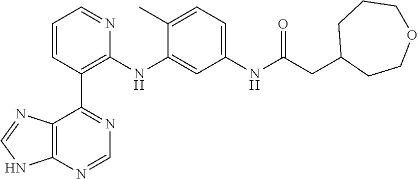 [SMILES: Cc1ccc(NC(=O)CC2CCCOCC2)cc1Nc1ncccc1-c1ncnc2[nH]cnc12] |
| IA-103 | 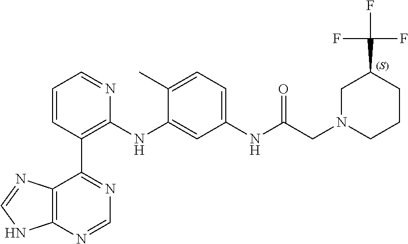 |
| IA-104 | 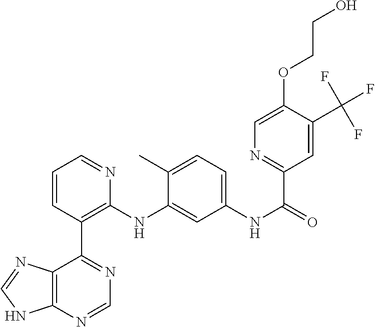 [SMILES: Cc1ccc(NC(=O)c2cc(C(F)(F)F)c(OCCO)cn2)cc1Nc1ncccc1-c1ncnc2[nH]cnc12] |
| IA-105 | 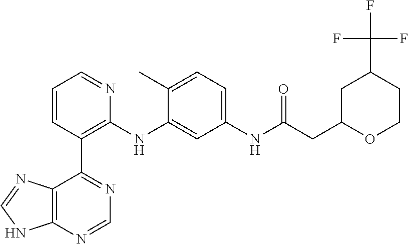 [SMILES: Cc1ccc(NC(=O)CC2CC(C(F)(F)F)CCO2)cc1Nc1ncccc1-c1ncnc2[nH]cnc12] |
| IA-106 | 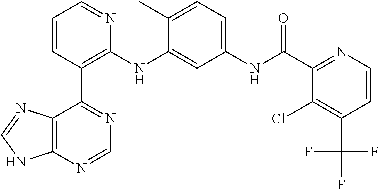 [SMILES: Cc1ccc(NC(=O)c2nccc(C(F)(F)F)c2Cl)cc1Nc1ncccc1-c1ncnc2[nH]cnc12] |
| IA-107 | 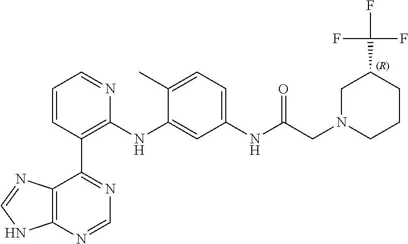 |
| IA-108 | 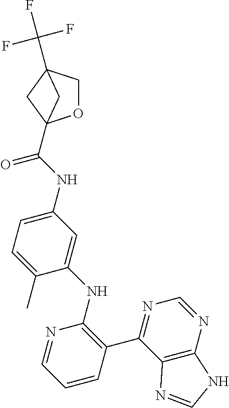 [SMILES: Cc1ccc(NC(=O)C23CC(C(F)(F)F)(CO2)C3)cc1Nc1ncccc1-c1ncnc2[nH]cnc12] |
| IA-109 | 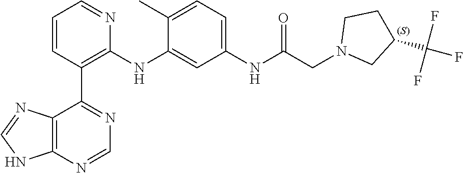 |
| IA-110 | 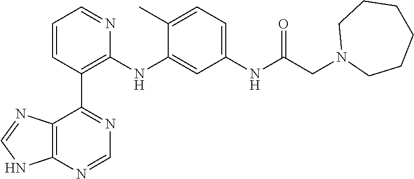 [SMILES: Cc1ccc(NC(=O)CN2CCCCCC2)cc1Nc1ncccc1-c1ncnc2[nH]cnc12] |
| IA-111 | 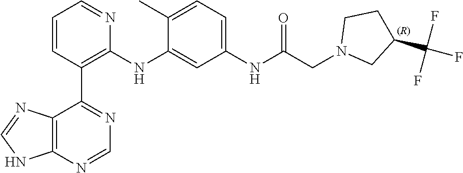 |
| IA-112 | 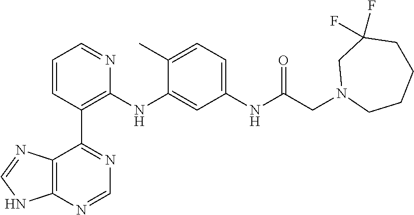 [SMILES: Cc1ccc(NC(=O)CN2CCCCC(F)(F)C2)cc1Nc1ncccc1-c1ncnc2[nH]cnc12] |
| IA-113 | 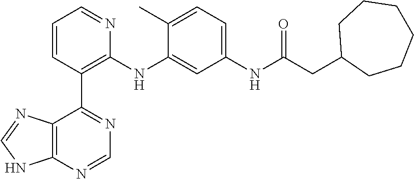 [SMILES: Cc1ccc(NC(=O)CC2CCCCCC2)cc1Nc1ncccc1-c1ncnc2[nH]cnc12] |
| IA-114 | 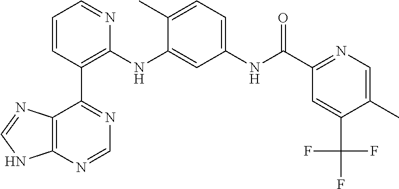 [SMILES: Cc1ccc(NC(=O)c2cc(C(F)(F)F)c(C)cn2)cc1Nc1ncccc1-c1ncnc2[nH]cnc12] |
| IA-115 | 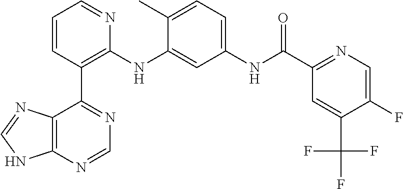 [SMILES: Cc1ccc(NC(=O)c2cc(C(F)(F)F)c(F)cn2)cc1Nc1ncccc1-c1ncnc2[nH]cnc12] |
| IA-116 | 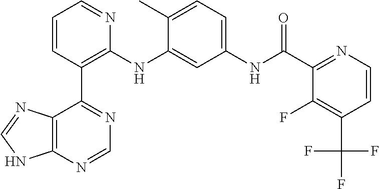 [SMILES: Cc1ccc(NC(=O)c2nccc(C(F)(F)F)c2F)cc1Nc1ncccc1-c1ncnc2[nH]cnc12] |
| IA-117 | 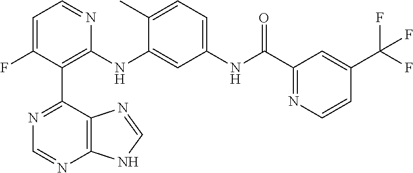 [SMILES: Cc1ccc(NC(=O)c2cc(C(F)(F)F)ccn2)cc1Nc1nccc(F)c1-c1ncnc2[nH]cnc12] |
| IA-118 | 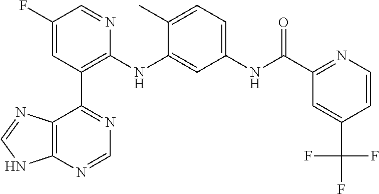 [SMILES: Cc1ccc(NC(=O)c2cc(C(F)(F)F)ccn2)cc1Nc1ncc(F)cc1-c1ncnc2[nH]cnc12] |
| IA-119 | 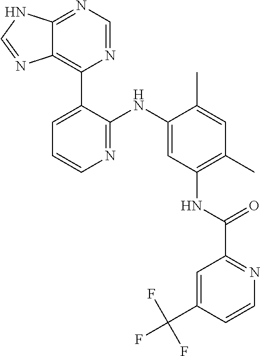 [SMILES: Cc1cc(C)c(Nc2ncccc2-c2ncnc3[nH]cnc23)cc1NC(=O)c1cc(C(F)(F)F)ccn1] |
| IA-120 | 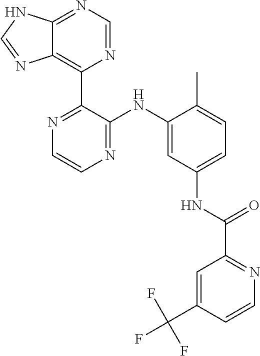 [SMILES: Cc1ccc(NC(=O)c2cc(C(F)(F)F)ccn2)cc1Nc1nccnc1-c1ncnc2[nH]cnc12] |
| IA-121 | 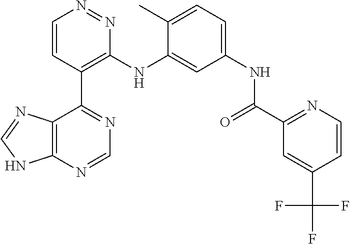 [SMILES: Cc1ccc(NC(=O)c2cc(C(F)(F)F)ccn2)cc1Nc1nnccc1-c1ncnc2[nH]cnc12] |
| IA-122 | 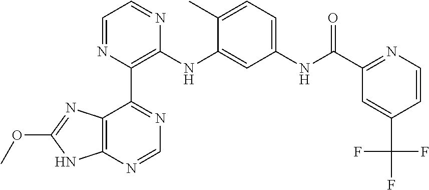 [SMILES: COc1nc2c(-c3nccnc3Nc3cc(NC(=O)c4cc(C(F)(F)F)ccn4)ccc3C)ncnc2[nH]1] |
| IA-123 | 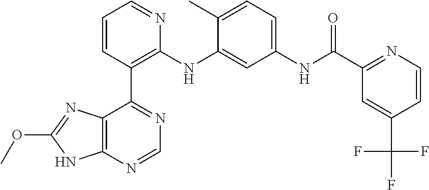 [SMILES: COc1nc2c(-c3cccnc3Nc3cc(NC(=O)c4cc(C(F)(F)F)ccn4)ccc3C)ncnc2[nH]1] |
| IA-124 | 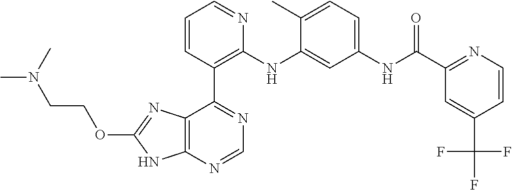 [SMILES: Cc1ccc(NC(=O)c2cc(C(F)(F)F)ccn2)cc1Nc1ncccc1-c1ncnc2[nH]c(OCCN(C)C)nc12] |
| IA-125 | 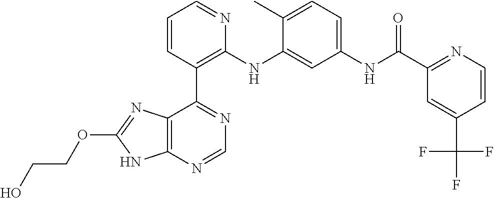 [SMILES: Cc1ccc(NC(=O)c2cc(C(F)(F)F)ccn2)cc1Nc1ncccc1-c1ncnc2[nH]c(OCCO)nc12] |
| IA-126 | 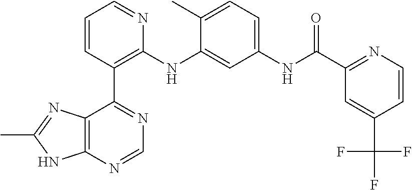 [SMILES: Cc1nc2c(-c3cccnc3Nc3cc(NC(=O)c4cc(C(F)(F)F)ccn4)ccc3C)ncnc2[nH]1] |
| IA-127 | 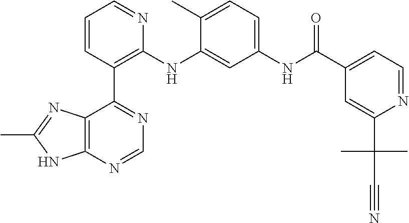 [SMILES: Cc1nc2c(-c3cccnc3Nc3cc(NC(=O)c4ccnc(C(C)(C)C#N)c4)ccc3C)ncnc2[nH]1] |
| IA-128 | 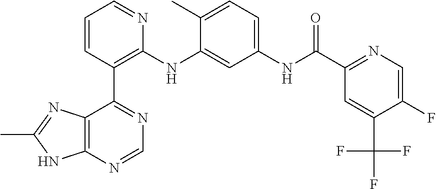 [SMILES: Cc1nc2c(-c3cccnc3Nc3cc(NC(=O)c4cc(C(F)(F)F)c(F)cn4)ccc3C)ncnc2[nH]1] |
| IA-129 | 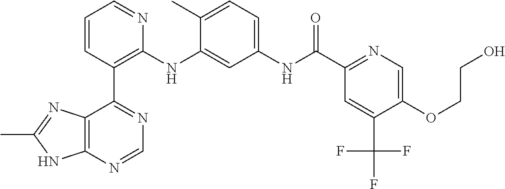 [SMILES: Cc1nc2c(-c3cccnc3Nc3cc(NC(=O)c4cc(C(F)(F)F)c(OCCO)cn4)ccc3C)ncnc2[nH]1] |
| IA-130 | 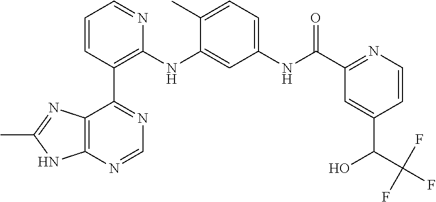 [SMILES: Cc1nc2c(-c3cccnc3Nc3cc(NC(=O)c4cc(C(O)C(F)(F)F)ccn4)ccc3C)ncnc2[nH]1] |
| IA-131 | 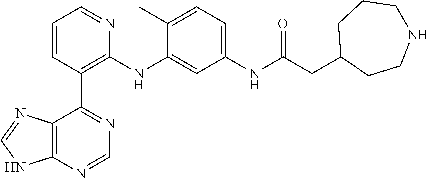 [SMILES: Cc1ccc(NC(=O)CC2CCCNCC2)cc1Nc1ncccc1-c1ncnc2[nH]cnc12] |
| IA-132 | 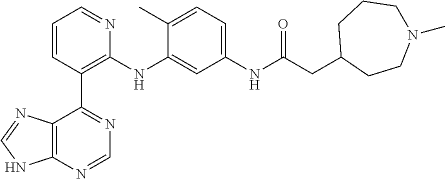 [SMILES: Cc1ccc(NC(=O)CC2CCCN(C)CC2)cc1Nc1ncccc1-c1ncnc2[nH]cnc12] |
| IA-133 | 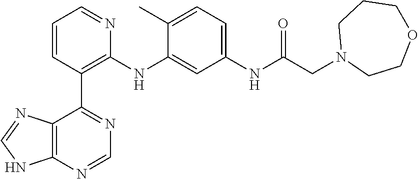 [SMILES: Cc1ccc(NC(=O)CN2CCCOCC2)cc1Nc1ncccc1-c1ncnc2[nH]cnc12] |
| IA-134 | 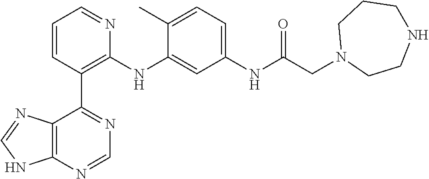 [SMILES: Cc1ccc(NC(=O)CN2CCCNCC2)cc1Nc1ncccc1-c1ncnc2[nH]cnc12] |
| IA-135 | 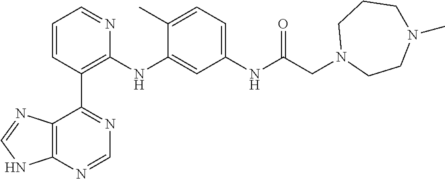 [SMILES: Cc1ccc(NC(=O)CN2CCCN(C)CC2)cc1Nc1ncccc1-c1ncnc2[nH]cnc12] |
| IA-136 | 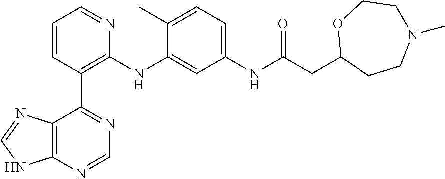 [SMILES: Cc1ccc(NC(=O)CC2CCN(C)CCO2)cc1Nc1ncccc1-c1ncnc2[nH]cnc12] |
| IA-137 | 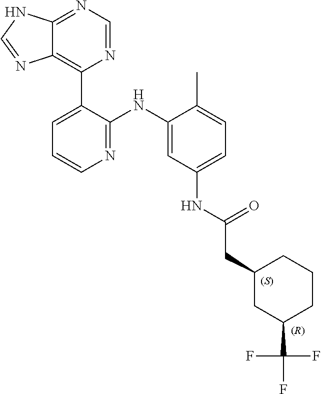 |
| IA-138 | 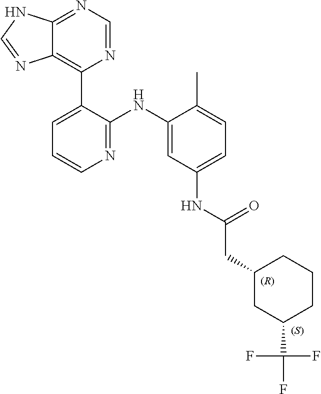 |
| IA-139 | 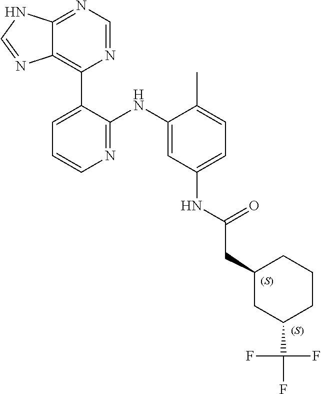 |
| IA-140 | 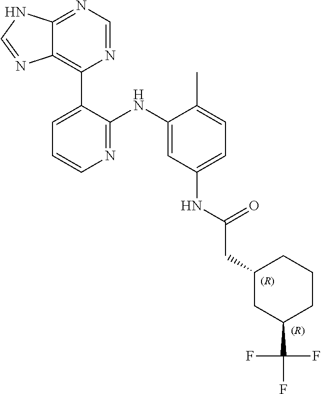 |
| IA-141 | 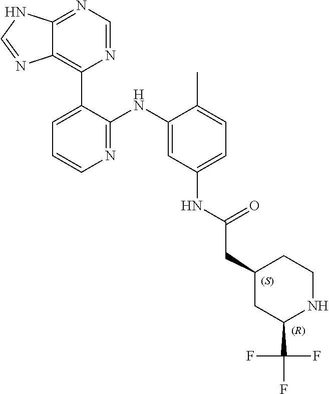 |
| IA-142 | 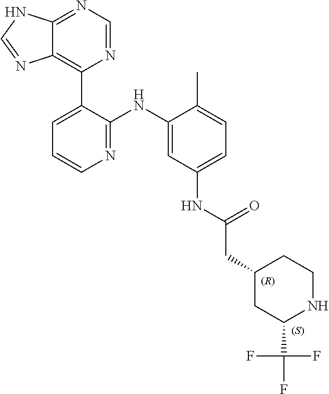 |
| IA-143 | 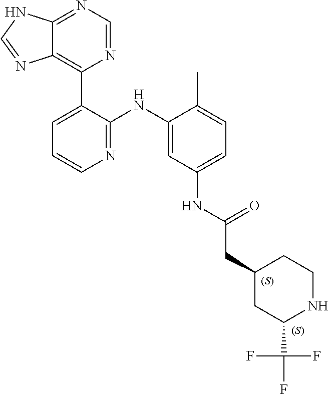 |
| IA-144 | 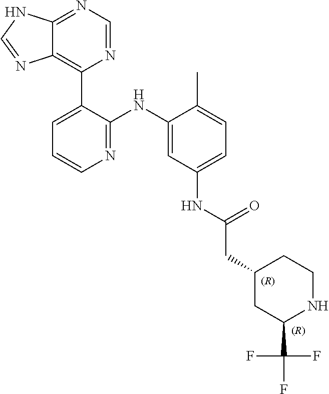 |
| IA-145 | 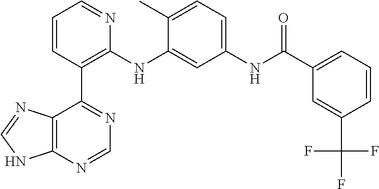 [SMILES: Cc1ccc(NC(=O)c2cccc(C(F)(F)F)c2)cc1Nc1ncccc1-c1ncnc2[nH]cnc12] |
| IA-146 | 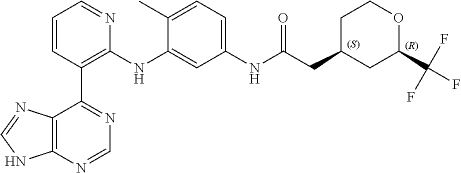 |
| IA-147 | 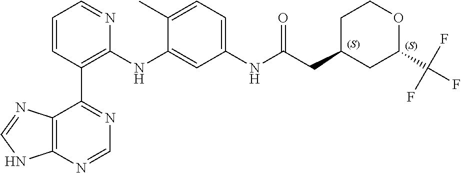 |
| IA-148 | 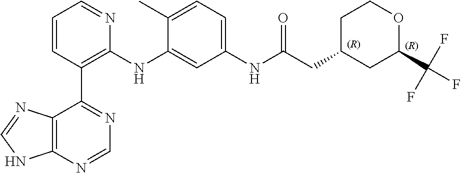 |
| IA-149 | 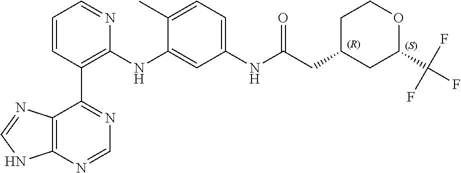 |
| IA-150 | 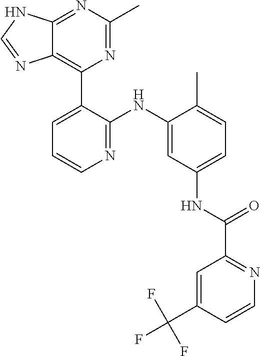 [SMILES: Cc1nc(-c2cccnc2Nc2cc(NC(=O)c3cc(C(F)(F)F)ccn3)ccc2C)c2nc[nH]c2n1] |
| IA-151 | 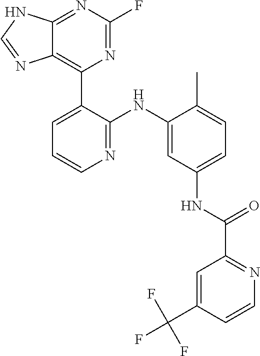 [SMILES: Cc1ccc(NC(=O)c2cc(C(F)(F)F)ccn2)cc1Nc1ncccc1-c1nc(F)nc2[nH]cnc12] |
| IA-152 | 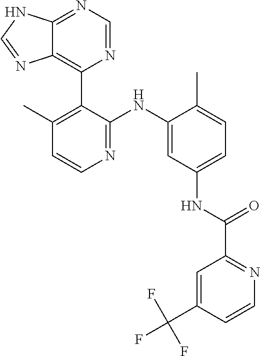 [SMILES: Cc1ccc(NC(=O)c2cc(C(F)(F)F)ccn2)cc1Nc1nccc(C)c1-c1ncnc2[nH]cnc12] |
| IA-153 | 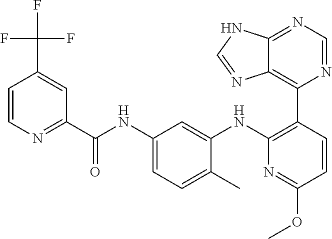 [SMILES: COc1ccc(-c2ncnc3[nH]cnc23)c(Nc2cc(NC(=O)c3cc(C(F)(F)F)ccn3)ccc2C)n1] |

TABLE II

| Compound | |
|---|---|
| No. | Structure |
| II-1 | 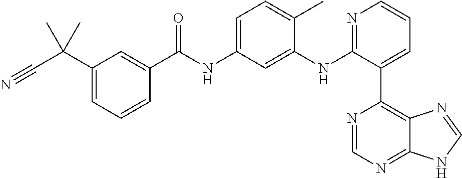 [SMILES: Cc1ccc(NC(=O)c2cccc(C(C)(C)C#N)c2)cc1Nc1ncccc1-c1ncnc2[nH]cnc12] |
| II-2 | 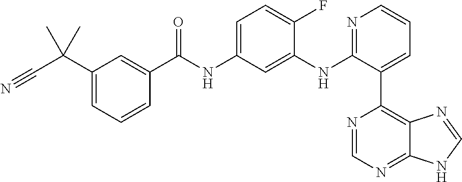 [SMILES: CC(C)(C#N)c1cccc(C(=O)Nc2ccc(F)c(Nc3ncccc3-c3ncnc4[nH]cnc34)c2)c1] |
| II-3 | 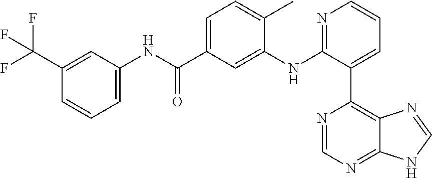 [SMILES: Cc1ccc(C(=O)Nc2cccc(C(F)(F)F)c2)cc1Nc1ncccc1-c1ncnc2[nH]cnc12] |

In some embodiments, the compound modulates activity against a Class II mutation or a Class III mutation.

In some embodiments, the compound modulates activity against a Class II mutation.

In some embodiments, the compound modulates activity against a Class III mutation.

In some embodiments, the compound exhibits an inhibition activity against a Class II mutation or a Class III mutation.

In some embodiments, the compound exhibits an inhibition activity against a Class II mutation.

In some embodiments, the compound exhibits an inhibition activity against a Class III mutation.

In some embodiments, the compound exhibits an inhibition activity against a Class II mutation or a Class III mutation that is higher than a comparable agent (e.g., encorafenib), as measured in IC₅₀ value.

In some embodiments, the compound exhibits an inhibition activity against a Class II mutation that is higher than a comparable agent (e.g., encorafenib), as measured in IC₅₀ value.

In some embodiments, the compound exhibits an inhibition activity against a Class III mutation that is higher than a comparable agent (e.g., encorafenib), as measured in IC₅₀ value.

In some embodiments, the compound exhibits an inhibition activity against a Class II mutation or a Class III mutation that is more than three-fold, more than four-fold, more than five-fold, or more than ten-fold higher than a comparable agent (e.g., encorafenib), as measured in IC₅₀ value.

In some embodiments, the compound exhibits an inhibition activity against a Class II mutation that is more than three-fold, more than four-fold, more than five-fold, or more than ten-fold higher than a comparable agent (e.g., encorafenib), as measured in IC₅₀ value.

In some embodiments, the compound exhibits an inhibition activity against a Class III mutation that is more than three-fold, more than four-fold, more than five-fold, or more than ten-fold higher than a comparable agent (e.g., encorafenib), as measured in IC₅₀ value.

In some aspects, the present disclosure provides a compound being an isotopic derivative (e.g., isotopically labeled compound) of any one of the compounds disclosed herein.

In some embodiments, the compound is an isotopic derivative of any one of the compounds described in Table I and Table IA, or a pharmaceutically acceptable salt thereof.

In some embodiments, the compound is an isotopic derivative of any one of the compounds described in Table I and Table IA.

In some embodiments, the compound is an isotopic derivative of any one of the compounds described in Table I, or a pharmaceutically acceptable salt thereof.

In some embodiments, the compound is an isotopic derivative of any one of the compounds described in Table I.

In some embodiments, the compound is an isotopic derivative of any one of the compounds described in Table IA, or a pharmaceutically acceptable salt thereof.

In some embodiments, the compound is an isotopic derivative of any one of the compounds described in Table IA.

In some embodiments, the compound is an isotopic derivative of any one of the compounds described in Table II, or a pharmaceutically acceptable salt thereof.

In some embodiments, the compound is an isotopic derivative of any one of the compounds described in Table II.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

In some embodiments, the compound is a deuterium labeled compound of any one of the compounds described in Table I and Table IA, or a pharmaceutically acceptable salt thereof.

In some embodiments, the compound is a deuterium labeled compound of any one of the compounds described in Table I and Table IA.

In some embodiments, the compound is a deuterium labeled compound of any one of the compounds described in Table I, or a pharmaceutically acceptable salt thereof.

In some embodiments, the compound is a deuterium labeled compound of any one of the compounds described in Table I.

In some embodiments, the compound is a deuterium labeled compound of any one of the compounds described in Table IA, or a pharmaceutically acceptable salt thereof.

In some embodiments, the compound is a deuterium labeled compound of any one of the compounds described in Table IA.

In some embodiments, the compound is a deuterium labeled compound of any one of the compounds described in Table II, or a pharmaceutically acceptable salt thereof.

In some embodiments, the compound is a deuterium labeled compound of any one of the compounds described in Table II.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the present disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulphuric, phosphoric, trifluoroacetic, formic, citric methane sulphonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of the present disclosure and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, trifluoroacetate, glutamate, glucuronate, glutarate, malate, maleate, succinate, fumarate, tartrate, tosylate, salicylate, lactate, naphthalenesulphonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of the present disclosure may exist in solvated as well as unsolvated forms such as, for example, hydrated forms. A suitable pharmaceutically acceptable solvate is, for example, a hydrate such as hemi-hydrate, a monohydrate, a di-hydrate or a tri-hydrate. It is to be understood that the disclosure encompasses all such solvated forms that possess inflammasome inhibitory activity.

It is also to be understood that certain compounds of the present disclosure may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of the present disclosure may exist in a number of different tautomeric forms and references to compounds of the present disclosure include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine nitroso/oxime thioketone/enethiol and nitro/aci-nitro

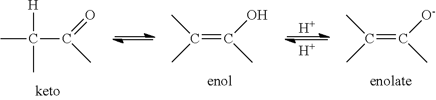

Compounds of the present disclosure containing an amine function may also form N-oxides. A reference herein to a compound disclosed herein that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of the present disclosure may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the sulphonylurea group in a compound of the any one of the Formulae disclosed herein. Accordingly, the present disclosure includes those compounds of the present disclosure as defined hereinbefore when made available by organic synthesis and when made available within the human or animal body by way of cleavage of a prodrug thereof. Accordingly, the present disclosure includes those compounds of the present disclosure that are produced by organic synthetic means and also such compounds that are produced in the human or animal body by way of metabolism of a precursor compound, that is a compound of the present disclosure may be a synthetically-produced compound or a metabolically-produced compound.

A suitable pharmaceutically acceptable prodrug of a compound of the present disclosure is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of the present disclosure that possesses a hydroxy group is, for example, an in vivo cleavable ester or ether thereof. An in vivo cleavable ester or ether of a compound of the present disclosure containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of the present disclosure that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of the present disclosure that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl, and 4-(C₁-C₄ alkyl)piperazin-1-ylmethyl.

The in vivo effects of a compound of the present disclosure may be exerted in part by one or more metabolites that are formed within the human or animal body after administration of a compound of the present disclosure. As stated hereinbefore, the in vivo effects of a compound of the present disclosure may also be exerted by way of metabolism of a precursor compound (a prodrug).

### Methods of Synthesis

In some aspects, the present disclosure provides a method of preparing a compound disclosed herein.

In some aspects, the present disclosure provides a method of preparing a compound, comprising one or more steps as described herein.

In some aspects, the present disclosure provides a compound obtainable by, or obtained by, or directly obtained by a method for preparing a compound described herein.

In some aspects, the present disclosure provides an intermediate being suitable for use in a method for preparing a compound described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of the present disclosure has been synthesized by any one of the processes defined herein, the processes may then further comprise the additional steps of: (i) removing any protecting groups present; (ii) converting the compound of the present disclosure into another compound of the present disclosure; (iii) forming a pharmaceutically acceptable salt, hydrate or solvate thereof; and/or (iv) forming a prodrug thereof.

The resultant compounds of the present disclosure can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulphoxides, such as dimethyl sulphoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Schemes I-III.

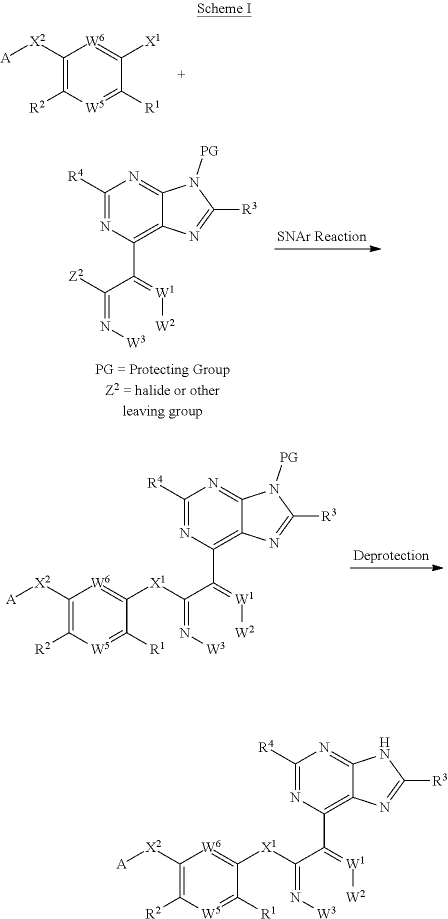

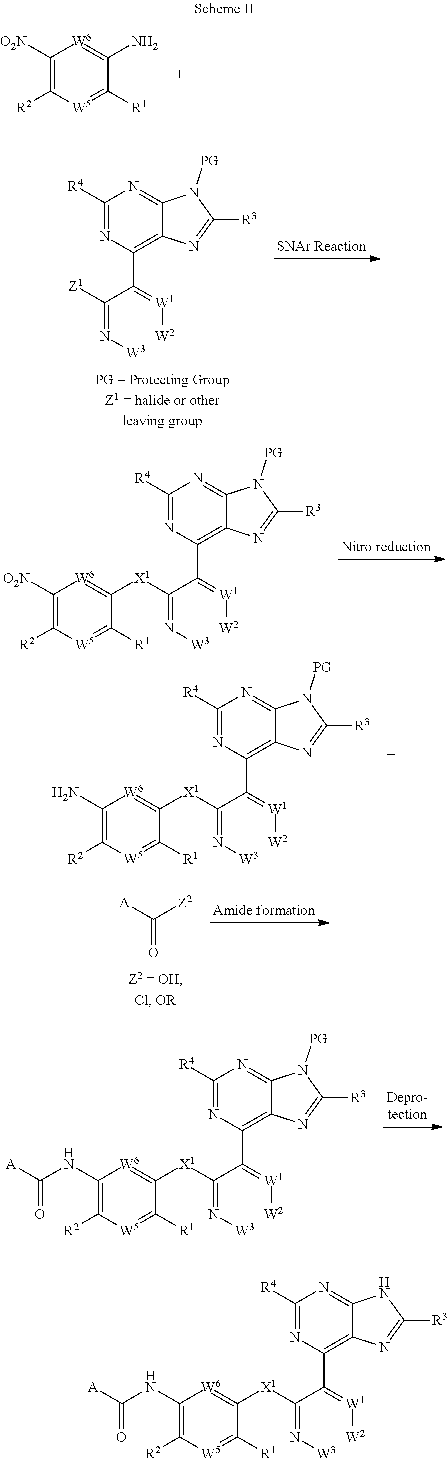

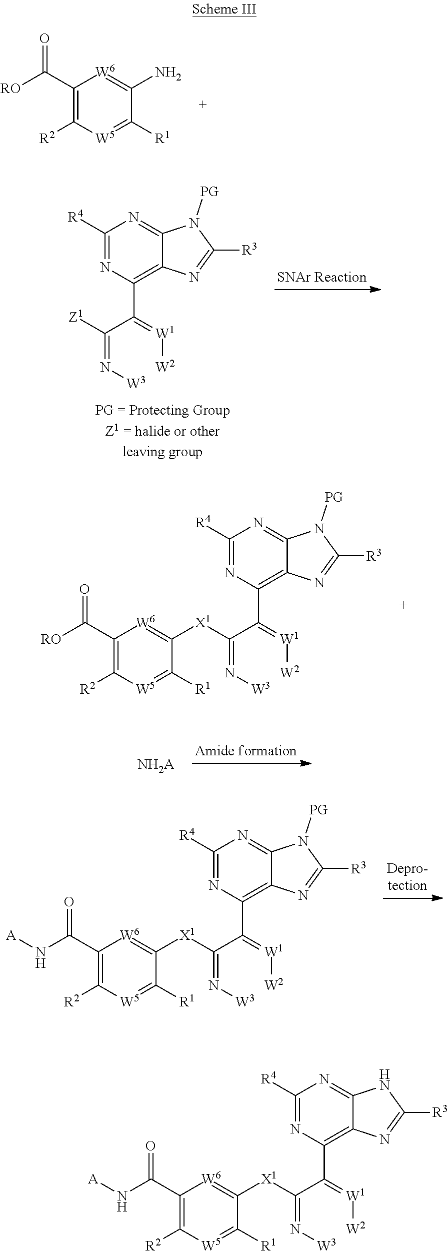

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments the biological assay may involve retroviral production.

In some embodiments, a fusion mutant (e.g., BRAF-KIAA1549) may be subcloned into a retroviral expression vector (e.g., pMXs-IRES-Blasticidin), wherein the retrovirus may be produced by transfection of cells (e.g., HEK 293T) with retroviral plasmids (e.g., retroviral BRAF mutant expression vector).

In some embodiments, the cells (e.g., HEK 293T) may be plated and incubated. In some embodiments, the retroviral plasmids (e.g., BRAF-KIAA fusion mutant) may added to a transfection reagent and then added to cells (e.g., HEK 293T), wherein the cells may be harvested.

In some embodiments, the biological assay may involve the generation of a fusion stable cell line (e.g., a BRAF-KIAA1549 fusion stable cell line).

In some embodiments, cells (e.g., BaF3) may be transduced with a viral supernatant (e.g., BRAF-KIAA1549 fusion viral supernatant) and the cells may be sampled for viability (e.g., by Luminescent Cell Viability Assay such as CellTiterGlo). In some embodiments, the fusion stable cell line may undergo cell banking and sequence confirmation (e.g., sanger sequencing).

In some embodiments, the biological assay is for cell proliferation.

In some embodiments, cells (e.g., BaF3 BRAF-KIAA1549 fusion cells) are suspended and dispensed in plates. In some embodiments, to determine the effect of compounds of the present disclosure on cell proliferation, the cells (e.g., BaF3 BRAF-KIAA1549 fusion cells) may be incubated in the presence of vehicle control (e.g., DMSO) or a compound of the present disclosure at varying concentrations and the inhibition of cell growth may be determined by luminescent quantification (e.g., of intracellular ATP content using CellTiterGlo), according to the manufacturers protocol. In some embodiments, to determine the IC₅₀ values, the vehicle-treated cells were normalized as viable cells and analyzed using a software (e.g., the CDD Vault (Collaborative Drug Discovery, Burlingame, CA) using an algorithm (e.g., the Levenberg-Marquardt algorithm; Levenberg, K., 1994; Marquardt, D., 1963).

### Pharmaceutical Compositions

In some aspects, the present disclosure provides a pharmaceutical composition comprising a compound of the present disclosure as an active ingredient.

In some embodiments, the present disclosure provides a pharmaceutical composition comprising a compound described herein and one or more pharmaceutically acceptable carriers or excipients. In some embodiments, the present disclosure provides a pharmaceutical composition comprising at least one compound selected from Table I, Table IA, and II. In some embodiments, the present disclosure provides a pharmaceutical composition comprising at least one compound selected from Table I and Table IA. In some embodiments, the present disclosure provides a pharmaceutical composition comprising at least one compound selected from Table I. In some embodiments, the present disclosure provides a pharmaceutical composition comprising at least one compound selected from Table IA. In some embodiments, the present disclosure provides a pharmaceutical composition comprising at least one compound selected from Table II.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S—β-CD), maltosyl-β-cyclodextrin, β-cyclodextrin sulphobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base-depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ¿-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent an inflammasome related condition referred to herein, slow its progression and/or reduce the symptoms associated with the condition.

The size of the dose for therapeutic or prophylactic purposes of a compound of the present disclosure will naturally vary according to the nature and severity of the conditions, the age and sex of the animal or patient and the route of administration, according to well-known principles of medicine.

### Methods of Use

In some aspects, the present disclosure provides methods of treating or preventing cancer in a subject, the method comprising administering to the subject at least one therapeutically effective amount of at least one compound of the present disclosure, or a pharmaceutically acceptable salt thereof.

In some aspects, the present disclosure provides methods of treating or preventing cancer in a subject, the method comprising administering to the subject a therapeutically effective amount of a compound of the present disclosure, or a pharmaceutically acceptable salt thereof.

In some aspects, the present disclosure provides methods of treating or preventing cancer in a subject, the method comprising administering to the subject a compound of the present disclosure, or a pharmaceutically acceptable salt thereof.

In some aspects, the present disclosure provides methods of treating cancer in a subject, the method comprising administering to the subject a therapeutically effective amount of at least one compound of the present disclosure, or a pharmaceutically acceptable salt thereof.

In some aspects, the present disclosure provides methods of treating cancer in a subject, the method comprising administering to the subject a therapeutically effective amount of a compound of the present disclosure, or a pharmaceutically acceptable salt thereof.

In some aspects, the present disclosure provides methods of treating cancer in a subject, the method comprising administering to the subject a compound of the present disclosure, or a pharmaceutically acceptable salt thereof. In some aspects, the present disclosure provides at least one compound of the present disclosure, or a pharmaceutically acceptable salt thereof, for treating or preventing cancer in a subject.

In some aspects, the present disclosure provides a compound of the present disclosure, or a pharmaceutically acceptable salt thereof, for treating or preventing cancer in a subject.

In some aspects, the present disclosure provides at least one compound of the present disclosure, or a pharmaceutically acceptable salt thereof, for treating cancer in a subject.

In some aspects, the present disclosure provides a compound of the present disclosure, or a pharmaceutically acceptable salt thereof, for treating cancer in a subject.

In some aspects, the present disclosure provides a use of at least one compound of the present disclosure, or a pharmaceutically acceptable salt thereof, in the manufacture of a medicament for treating or preventing cancer in a subject.

In some aspects, the present disclosure provides a use of a compound of the present disclosure, or a pharmaceutically acceptable salt thereof, in the manufacture of a medicament for treating or preventing cancer in a subject.

In some aspects, the present disclosure provides a use of at least one compound of the present disclosure, or a pharmaceutically acceptable salt thereof, in the manufacture of a medicament for treating cancer in a subject.

In some aspects, the present disclosure provides a use of a compound of the present disclosure, or a pharmaceutically acceptable salt thereof, in the manufacture of a medicament for treating cancer in a subject.

In some aspects, the present disclosure provides a use of at least one compound of the present disclosure, or a pharmaceutically acceptable salt thereof, for treating or preventing cancer in a subject.

In some aspects, the present disclosure provides a use of a compound of the present disclosure, or a pharmaceutically acceptable salt thereof, for treating or preventing cancer in a subject.

In some aspects, the present disclosure provides a use of at least one compound of the present disclosure, or a pharmaceutically acceptable salt thereof, for treating cancer in a subject.

In some aspects, the present disclosure provides a use of a compound of the present disclosure, or a pharmaceutically acceptable salt thereof, for treating cancer in a subject.

In some aspects, the present disclosure provides methods of treating or preventing cancer in a subject, the method comprising administering to the subject at least one therapeutically effective amount of a pharmaceutical composition comprising at least one compound of the present disclosure, or a pharmaceutically acceptable salt thereof.

In some aspects, the present disclosure provides methods of treating or preventing cancer in a subject, the method comprising administering to the subject a therapeutically effective amount of a pharmaceutical composition comprising at least one compound of the present disclosure, or a pharmaceutically acceptable salt thereof.

In some aspects, the present disclosure provides methods of treating or preventing cancer in a subject, the method comprising administering to the subject a therapeutically effective amount of a pharmaceutical composition comprising a compound of the present disclosure, or a pharmaceutically acceptable salt thereof.

In some aspects, the present disclosure provides methods of treating or preventing cancer in a subject, the method comprising administering to the subject a pharmaceutical composition comprising a compound of the present disclosure, or a pharmaceutically acceptable salt thereof.

In some aspects, the present disclosure provides methods of treating cancer in a subject, the method comprising administering to the subject a therapeutically effective amount of a pharmaceutical composition comprising at least one compound of the present disclosure, or a pharmaceutically acceptable salt thereof.

In some aspects, the present disclosure provides methods of treating cancer in a subject, the method comprising administering to the subject a therapeutically effective amount of a pharmaceutical composition comprising a compound of the present disclosure, or a pharmaceutically acceptable salt thereof.

In some aspects, the present disclosure provides methods of treating cancer in a subject, the method comprising administering to the subject a pharmaceutical composition comprising a compound of the present disclosure, or a pharmaceutically acceptable salt thereof.

In some aspects, the present disclosure provides a pharmaceutical composition comprising at least one compound of the present disclosure, or a pharmaceutically acceptable salt thereof, for treating or preventing cancer in a subject.

In some aspects, the present disclosure provides a pharmaceutical composition comprising a compound of the present disclosure, or a pharmaceutically acceptable salt thereof, for treating or preventing cancer in a subject.

In some aspects, the present disclosure provides a pharmaceutical composition comprising at least one compound of the present disclosure, or a pharmaceutically acceptable salt thereof, for treating cancer in a subject.

In some aspects, the present disclosure provides a pharmaceutical composition comprising a compound of the present disclosure, or a pharmaceutically acceptable salt thereof, for treating cancer in a subject.

In some aspects, the present disclosure provides a use of a pharmaceutical composition comprising at least one compound of the present disclosure, or a pharmaceutically acceptable salt thereof, in the manufacture of a medicament for treating or preventing cancer in a subject.

In some aspects, the present disclosure provides a use of a pharmaceutical composition comprising a compound of the present disclosure, or a pharmaceutically acceptable salt thereof, in the manufacture of a medicament for treating or preventing cancer in a subject.

In some aspects, the present disclosure provides a use of a pharmaceutical composition comprising at least one compound of the present disclosure, or a pharmaceutically acceptable salt thereof, in the manufacture of a medicament for treating cancer in a subject.

In some aspects, the present disclosure provides a use of a pharmaceutical composition comprising a compound of the present disclosure, or a pharmaceutically acceptable salt thereof, in the manufacture of a medicament for treating cancer in a subject.

In some aspects, the present disclosure provides a use of a pharmaceutical composition comprising at least one compound of the present disclosure, or a pharmaceutically acceptable salt thereof, for treating or preventing cancer in a subject.

In some aspects, the present disclosure provides a use of a pharmaceutical composition comprising a compound of the present disclosure, or a pharmaceutically acceptable salt thereof, for treating or preventing cancer in a subject.

In some aspects, the present disclosure provides a use of a pharmaceutical composition comprising at least one compound of the present disclosure, or a pharmaceutically acceptable salt thereof, for treating cancer in a subject.

In some aspects, the present disclosure provides a use of a pharmaceutical composition comprising a compound of the present disclosure, or a pharmaceutically acceptable salt thereof, for treating cancer in a subject.

In some aspects, the present disclosure provides methods of treating or preventing cancer in a subject, the method comprising administering to the subject at least one therapeutically effective amount of a pharmaceutical kit comprising at least one compound of the present disclosure, or a pharmaceutically acceptable salt thereof.

In some aspects, the present disclosure provides methods of treating or preventing cancer in a subject, the method comprising administering to the subject a therapeutically effective amount of a pharmaceutical kit comprising at least one compound of the present disclosure, or a pharmaceutically acceptable salt thereof.

In some aspects, the present disclosure provides methods of treating or preventing cancer in a subject, the method comprising administering to the subject a therapeutically effective amount of a pharmaceutical kit comprising a compound of the present disclosure, or a pharmaceutically acceptable salt thereof.

In some aspects, the present disclosure provides methods of treating or preventing cancer in a subject, the method comprising administering to the subject a pharmaceutical kit comprising a compound of the present disclosure, or a pharmaceutically acceptable salt thereof.

In some aspects, the present disclosure provides methods of treating cancer in a subject, the method comprising administering to the subject a therapeutically effective amount of a pharmaceutical kit comprising at least one compound of the present disclosure, or a pharmaceutically acceptable salt thereof.

In some aspects, the present disclosure provides methods of treating cancer in a subject, the method comprising administering to the subject a therapeutically effective amount of a pharmaceutical kit comprising a compound of the present disclosure, or a pharmaceutically acceptable salt thereof.

In some aspects, the present disclosure provides methods of treating cancer in a subject, the method comprising administering to the subject a pharmaceutical kit comprising a compound of the present disclosure, or a pharmaceutically acceptable salt thereof.

In some aspects, the present disclosure provides a pharmaceutical kit comprising at least one compound of the present disclosure, or a pharmaceutically acceptable salt thereof, for treating or preventing cancer in a subject.

In some aspects, the present disclosure provides a pharmaceutical kit comprising a compound of the present disclosure, or a pharmaceutically acceptable salt thereof, for treating or preventing cancer in a subject.

In some aspects, the present disclosure provides a pharmaceutical kit comprising at least one compound of the present disclosure, or a pharmaceutically acceptable salt thereof, for treating cancer in a subject.

In some aspects, the present disclosure provides a pharmaceutical kit comprising a compound of the present disclosure, or a pharmaceutically acceptable salt thereof, for treating cancer in a subject.

In some aspects, the present disclosure provides a use of a pharmaceutical kit comprising at least one compound of the present disclosure, or a pharmaceutically acceptable salt thereof, in the manufacture of a medicament for treating or preventing cancer in a subject.

In some aspects, the present disclosure provides a use of a pharmaceutical kit comprising a compound of the present disclosure, or a pharmaceutically acceptable salt thereof, in the manufacture of a medicament for treating or preventing cancer in a subject.

In some aspects, the present disclosure provides a use of a pharmaceutical kit comprising at least one compound of the present disclosure, or a pharmaceutically acceptable salt thereof, in the manufacture of a medicament for treating cancer in a subject.

In some aspects, the present disclosure provides a use of a pharmaceutical kit comprising a compound of the present disclosure, or a pharmaceutically acceptable salt thereof, in the manufacture of a medicament for treating cancer in a subject.

In some aspects, the present disclosure provides a use of a pharmaceutical kit comprising at least one compound of the present disclosure, or a pharmaceutically acceptable salt thereof, for treating or preventing cancer in a subject.

In some aspects, the present disclosure provides a use of a pharmaceutical kit comprising a compound of the present disclosure, or a pharmaceutically acceptable salt thereof, for treating or preventing cancer in a subject.

In some aspects, the present disclosure provides a use of a pharmaceutical kit comprising at least one compound of the present disclosure, or a pharmaceutically acceptable salt thereof, for treating cancer in a subject.

In some aspects, the present disclosure provides a use of a pharmaceutical kit comprising a compound of the present disclosure, or a pharmaceutically acceptable salt thereof, for treating cancer in a subject.

BRAF is a human gene located on the long arm of chromosome 7 (7q34) that encodes for a protein known as B-Raf. B-Raf is a serine/threonine kinase that resides in the cytoplasm of cells. B-Raf is an effector molecule within the mitogen-activated protein kinase (MAPK)/extracellular signal-regulated kinase (ERK) signaling pathway, a pathway that is known to regulate various cellular processes including, but not limited to, growth, proliferation, differentiation and apoptosis.

Briefly, and as would be appreciated by the skilled artisan, in the MAPK/ERK signaling pathway, certain external stimuli, such as growth factors, activate receptors located on the cellular membrane, including receptor tyrosine kinases (RTKs). These receptors in turn activate RAS, causing an exchange of GDP to GTP, thereby producing RAS-GTP. RAS-GTP then activates a Mitogen Activated Protein kinase kinase kinase (MAPKKK or MAP3K). The activated MAPKKK then activates a MAP kinase kinase (MAPKK). The activated MAPKK then activates a MAP kinase (MAPK). Activated MAPK then activates downstream effectors, including transcriptions factors, causing changes in gene expression, thereby regulating the various cellular processes described above, including, but not limited to, cellular growth, proliferation, differentiation and apoptosis.

Examples of MAPKKKs include members of the rapidly accelerated fibrosarcoma (Raf) family, including Raf-1 (also known as C-Raf), B-Raf and A-Raf.

Raf proteins, including B-raf, have three conserved domains denoted conserved region 1 (CR1), conserved region 2 (CR2) and conserved region 3 (CR3). CR1 is an autoinhibitory domain that inhibits the Raf protein's kinase domain (CR3). CR1 includes a binding site for RAS-GTP's effector domain. Upon CR1 binding to RAS-GTP's effector domain, CR1 releases the CR3; relieving autoinhibition of the kinase domain. CR2 is flexible linker that acts as a hinge to connected CR1 and CR3. CR3 is an enzymatic kinase domain.

In its active form, B-Raf forms a dimer and functions as a serine/threonine-specific protein kinase. Under activating conditions, the regulatory protein 14-3-3 is displaced from CR2, of B-Raf, allowing for a de-clamping of CR1 and CR2. Additionally, RAS-GTP binds to CR1 of B-Raf, causing CR1 to release CR3. The overall effect is that the autoinhibition of the kinase domain of B-Raf is relieved. Subsequently, B-Raf is phosphorylated at T599 and S602, which results in the kinase domain switching to the active confirmation. Dimerization can then occur, which further stabilizes the active form of B-Raf.

Mutations in the BRAF gene have been implicated in a variety of different cancers, including, but not limited to, melanoma, non-Hodgkin's lymphoma, colorectal cancer, papillary thyroid carcinoma, non small cell lung cancer (NSCLC) and glioblastoma. As of 2019; approximately 200 BRAF-mutant alleles have been identified in human tumors, with at least 30 distinct mutations having been functionally characterized. BRAF mutations are typically categorized into one of three classes based on the mutations effect on B-Raf activity.

Class I (or Class 1) mutations are mutations that result in the expression of mutant B-Raf that can become active in the monomeric form, independent of RAS activity. That is, Class I mutations in BRAF yield the expression of B-Raf proteins that are RAS-independent, active monomers. These RAS-independent, active monomers typically demonstrate elevated levels of kinase activity.

Class II (or Class 2) mutations are mutations that result in the expression of mutant B-Raf that can form active dimers independent of RAS. That is, Class II mutations in BRAF yield the expression of B-Raf proteins that are RAS-independent, active dimers. These RAS-independent, active dimers also display intermediate to high levels of kinase activity, but their activity levels are typically lower compared to the RAS-independent, active monomers produced by Class I BRAF mutations.

Class III (or Class 3) mutations are mutations that result in the expression of mutant B-Raf that are RAS dependent (i.e. must be activated by RAS-GTP) and that can form heterodimers with other MAPK proteins such as C-Raf. Class III mutations in BRAF typically yield B-Raf with low or impaired kinase activity.

As would be appreciated by the skilled artisan, since Class I BRAF mutations and Class II BRAF mutations are RAS-independent, the mutant B-Raf proteins harboring Class I or Class II mutations are uncoupled to any upstream signals, resulting in constitutive activation that can result in unchecked cellular growth and eventually oncogenic proliferation. In some embodiments, the subject is a mammal.

In some embodiments, the subject is a human.

In some embodiments, the cancer is characterized by at least one oncogenic mutation in the BRAF gene.

It is understood that a cancer that is characterized by at least one oncogenic mutation in the BRAF gene is a cancer that is typically associated with at least one oncogenic mutation in the BRAF gene, including, but not limited to, cancers whose primary oncogenic activity is thought to be driven by the at least one oncogenic mutation in the BRAF gene.

In some embodiments, the cancer is characterized by at least one oncogenic variant of B-Raf.

It is understood that a cancer that is characterized by least one oncogenic variant of B-Raf is a cancer that is typically associated with at least one oncogenic variant of B-Raf, including, but not limited to, cancers whose primary oncogenic activity is thought to be driven by the at least one oncogenic variant of B-Raf.

It is understood that an oncogenic variant of B-Raf is a B-Raf protein that comprises at least one oncogenic mutation and that is produced as the result of the expression of a BRAF gene that comprises at least one oncogenic mutation.

In some embodiments, the subject has at least one oncogenic mutation in the BRAF gene.

In some embodiments, the subject has at least one tumor and/or cancerous cell that expresses an oncogenic variant of B-Raf.

As would be appreciated by the skilled artisan, in the context of a gene (e.g. BRAF), an oncogenic mutation can include, but is not limited to a mutation that results in the substitution of one amino acid for another at a specific position within B-Raf, a mutation that results in the substitution of one or more amino acids for one or more amino acids between two specific positions within B-Raf, a mutation that results in an insertion of one or more amino acids between two positions within B-Raf, a mutation that results in the deletion of one more amino acids between two positions within B-Raf, and mutation that results in a fusion of B-Raf, or portion thereof, with another protein, or portion thereof, or any combination thereof. As would be appreciated by the skilled artisan, in the context of a gene, an oncogenic mutation can include, but is not limited to, a missense mutation, a nonsynonymous mutation, an insertion of one or more nucleotides, a deletion of one or more nucleotides, an inversion and a deletion-insertion. As would be appreciated by the skilled artisan, in the context of a gene (e.g. BRAF), the gene can have one or more of the aforementioned types of oncogenic mutations, including combinations of different types of oncogenic mutations.

As would be appreciated by the skilled artisan, in the context of a protein (e.g. B-Raf), an oncogenic mutation can include, but is not limited to, the substitution of one amino acid for another at a specific position within B-Raf, the substitution of one or more amino acids for one or more amino acids between two specific positions within B-Raf, an insertion of one or more amino acids between two positions within B-Raf, a deletion of one more amino acids between two positions within B-Raf, and a fusion of B-Raf, or portion thereof, with another protein, or portion thereof, or any combination thereof. As would be appreciated by the skilled artisan, in the context of a protein (e.g. B-Raf), the protein can have one or more of the aforementioned types of oncogenic mutations, including combinations of different types of oncogenic mutations.

In some embodiments, an oncogenic mutation of B-Raf can be any of the B-Raf mutations put forth in Table 1a. An oncogenic variant of B-Raf can comprise any combination of the oncogenic mutations put forth in Table 1a. In a non-limiting example, an oncogenic variant of β-Raf can comprise the oncogenic mutations K601E and S363F.

TABLE 1a
B-Raf mutations (numbering corresponding to SEQ ID NO: 1)

| | | | |
|---|---|---|---|
| S363F | G464V | N581S | V600K |
| F247L | G466E | D594G | V600R |
| | G466V | D594N | K601E |
| G464R | S467L | L597Q | K601N |
| L597R | G469A | L597V | |
| G469R | G469V | V600E | L485-P490 Deletion |
| G466A | L584F | K601T | L485-P490 > Y |
| | | | (L485-P490Y) |
| KIAA Fusion | SND-1 Fusion | SKAP2 Fusion | 487-492 Deletion |
| (KIAA1549 | | | |
| fusion) | | | |
| CUL-1 Fusion | | | |

As would be appreciated by the skilled Artisan, L485-P490>Y and L485-P490Y refers to the substitution residues L485 through P490 of B-Raf (SEQ ID NO: 1) with a Tyrosine (Y) residue.

In some embodiments, an oncogenic mutation of B-Raf can comprise a deletion of any combination of one or more amino acids between L485 and P490 of B-Raf (SEQ ID NO: 1). In some embodiments, an oncogenic mutation of B-Raf can comprise a deletion of any combination of one or more amino acids between L485 and Q494 of B-Raf (SEQ ID NO: 1). In some embodiments, an oncogenic mutation of B-Raf can comprise a deletion of any combination of one or more amino acids between A481 and Q494 of B-Raf (SEQ ID NO: 1). In some embodiments, an oncogenic mutation of B-Raf can comprise a deletion of any combination of one or more amino acids between K475 and N500 of B-Raf (SEQ ID NO: 1). In some embodiments, any of the preceding deletions can further comprise any combination of one or more substitutions and/or insertions within the range of residues indicated.

A wild type B-Raf sequence of the present disclosure may comprise, consist essentially of, or consist of the amino acid sequence of:

| (SEQ ID NO: 1) | |
|---|---|
| 1 | MAALSGGGGG GAEPGQALFN GDMEPEAGAG AGAAASSAAD PAIPEEVWNI |
| 51 | KQMIKLTQEH IEALLDKFGG EHNPPSIYLE AYEEYTSKLD ALQQREQQLL |
| 101 | ESLGNGTDFS VSSSASMDTV TSSSSSSLSV LPSSLSVFQN PTDVARSNPK |
| 151 | SPQKPIVRVF LPNKQRTVVP ARCGVTVRDS LKKALMMRGL IPECCAVYRI |
| 201 | QDGEKKPIGW DTDISWLTGE ELHVEVLENV PLTTHNFVRK TFFTLAFCDF |
| 251 | CRKLLFQGFR CQTCGYKFHQ RCSTEVPLMC VNYDQLDLLF VSKFFEHHPI |
| 301 | PQEEASLAET ALTSGSSPSA PASDSIGPQI LTSPSPSKSI PIPQPFRPAD |
| 351 | EDHRNQFGQR DRSSSAPNVH INTIEPVNID DLIRDQGFRG DGGSTTGLSA |
| 401 | TPPASLPGSL TNVKALQKSP GPQRERKSSS SSEDRNRMKT LGRRDSSDDW |
| 451 | EIPDGQITVG QRIGSGSFGT VYKGKWHGDV AVKMLNVTAP TPQQLQAFKN |
| 501 | EVGVLRKTRH VNILLFMGYS TKPQLAIVTQ WCEGSSLYHH LHIIETKFEM |
| 551 | IKLIDIARQT AQGMDYLHAK SIIHRDLKSN NIFLHEDLTV KIGDFGLATV |
| 601 | KSRWSGSHQF EQLSGSILWM APEVIRMQDK NPYSFQSDVY AFGIVLYELM |
| 651 | TGQLPYSNIN NRDQIIFMVG RGYLSPDLSK VRSNCPKAMK RLMAECLKKK |
| 701 | RDERPLFPQI LASIELLARS LPKIHRSASE PSLNRAGFQT EDFSLYACAS |
| 751 | PKTPIQAGGY GAFPVH |

In some embodiments, the oncogenic mutation is a class I mutation. Accordingly, in some embodiments, the oncogenic variant of B-Raf comprises a class I mutation.

In some embodiments, the oncogenic mutation is a class II mutation. Accordingly, in some embodiments, the oncogenic variant of B-Raf comprises a class II mutation.

In some embodiments, the oncogenic mutation is a class III mutation. Accordingly, in some embodiments, the oncogenic variant of B-Raf comprises a class III mutation.

In some embodiments, the oncogenic variant of B-Raf can be any of the B-Raf variants put forth in Table 1b.

TABLE 1b
B-Raf oncogenic variants (numbering corresponding to SEQ ID NO: 1)

| | | | |
|---|---|---|---|
| B-Raf-K601E-S363F | B-Raf-G464V | B-Raf-N581S | B-Raf-V600K |
| B-Raf-F247L | B-Raf-G466E | B-Raf-D594G | B-Raf-V600R |
| | B-Raf-G466V | B-Raf-D594N | B-Raf-K601E |
| B-Raf-G464R | B-Raf-S467L | B-Raf-L597Q | B-Raf-K601N |
| B-Raf-L597R | B-Raf-G469A | B-Raf-L597V | |
| B-Raf-G469R | B-Raf-G469V | B-Raf-V600E | B-RAF-L485-P490 |
| | | | Deletion |
| B-Raf-G466A | B-Raf-L584F | B-Raf-K601T | B-Raf-L485-P490 > Y |
| | | | (B-Raf-L485-P490Y) |
| B-Raf-KIAA Fusion | B-Raf-SND-1 | B-Raf-SKAP2 | B-Raf-487-492 |
| (B-Raf-KIAA1549 | Fusion | Fusion | Deletion |
| fusion) | | | |
| B-Raf-CUL-1 Fusion | | | |

In some embodiments, an oncogenic variant of B-Raf can comprise a deletion of any combination of one or more amino acids between L485 and P490 of B-Raf (SEQ ID NO: 1). In some embodiments, an oncogenic variant of B-Raf can comprise a deletion of any combination of one or more amino acids between L485 and Q494 of B-Raf (SEQ ID NO: 1). In some embodiments, an oncogenic variant of B-Raf can comprise a deletion of any combination of one or more amino acids between A481 and Q494 of B-Raf (SEQ ID NO: 1). In some embodiments, an oncogenic variant of B-Raf can comprise a deletion of any combination of one or more amino acids between K475 and N500 of B-Raf (SEQ ID NO: 1). In some embodiments, any of the preceding deletions can further comprise any combination of one or more substitutions and/or insertions within the range of residues indicated.

In some embodiments, a subject has at least one tumor and/or cancerous cell that expresses an oncogenic variant of B-Raf and an N-Ras protein comprising at least one mutation. In some as embodiments, an N-Ras protein comprising at least one mutation can be N-Ras-G12D, N-Ras-Q61K, and/or N-Ras-Q61R. In a non-limiting example, a subject can have at least one tumor and/or cancerous cell that expresses B-Raf-D594G and N-Ras-G12D.

In some embodiments, the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

In some embodiments, the cancer is adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Pheochromocytoma, Prostate cancer, Pseudomyxoma peritonei, Rectal cancer. Retinoblastoma, Salivary gland cancer, Secondary’ cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland, Uterine cancer. Vaginal cancer, Vulval cancer, Wilms' tumor, Womb cancer and Gynaecological cancer. Examples of cancer also include, but are not limited to, Hematologic malignancies, Lymphoma, Cutaneous T-cell lymphoma, Peripheral T-cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

In some embodiments, the cancer is a hematological cancer.

In some embodiments, the cancer is a solid cancer (also referred to as a solid malignancy or a solid tumor).

In some embodiments, the cancer is melanoma, breast cancer, head and neck cancer, esophagogastric cancer, stomach and small intestine cancer, lung cancer, mesothelioma, hepatobiliary cancer, pancreatic cancer, kidney cancer, colorectal cancer, endometrial cancer, cervical cancer, ovarian cancer, bladder cancer, prostate cancer, soft tissue sarcoma, CNS and brain cancer, or thyroid cancer.

In some embodiments, the cancer is non-small cell lung cancer (NSCLC), colorectal cancer, melanoma, thyroid cancer, histiocytosis, small bowel cancer, gastrointestinal neuroendocrine cancer, carcinoma of unknown primary, non-melanoma skin cancer, prostate cancer, gastric cancer, non-Hodgkin's lymphoma, papillary thyroid carcinoma or glioblastoma.

In some embodiments, the administration does not induce paradoxical activation of wild-type B-Raf.

In some embodiments, the administration does not substantially increase the amount of p-ERK in the subject.

In some embodiments, the administration results in an amount of p-ERK in the subject that is at least about 10% lower, at least about 20% lower, at least about 30% lower, at least about 40% lower, at least about 50% lower, at least about 60% lower, at least about 70% lower, at least about 80% lower, at least about 90% lower, or at least about 95% lower as compared to a comparable subject being administered with vemurafenib or encorafenib.

In some embodiments, the administration results in an amount of p-ERK in the subject that is at least about 10% lower, at least about 20% lower, at least about 30% lower, at least about 40% lower, at least about 50% lower, at least about 60% lower, at least about 70% lower, at least about 80% lower, at least about 90% lower, or at least about 95% lower as compared to a comparable subject without administration.

In some embodiments, the administration reduces the tumor volume in the subject by at least about 10% lower, at least about 20%, at least about 30%, at least about 40%, at least about 50%, at least about 60%, at least about 70%, at least about 80%, at least about 90%, at least about 95%, at least about 98%, or at least about 99%.

### Definitions

Unless otherwise stated, the following terms used in the specification and claims have the following meanings set out below.

As would be appreciated by the skilled artisan, the BRAF gene is commonly referred to as one of BRAF, β-RAF1, BRAF1, NS7, RAFB1, B-Raf proto-oncogene, proto-oncogene B-raf, v-Raf murine sarcoma viral oncogene homolog B, and v-Raf murine sarcoma viral oncogene homolog B1. Thus, these terms are used herein interchangeably to refer to the BRAF gene.

As would be appreciated by the skilled artisan, the B-Raf protein, encoded by the BRAF gene, is commonly referred to as one of BRAF, B-Raf, serine/threonine-protein kinase B-Raf, proto-oncogene B-Raf, p94 and v-Raf murine sarcoma viral oncogene homolog B1. Thus, these terms are used herein interchangeably to refer to the B-Raf gene.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

It is to be understood that a compound of the present disclosure may be depicted in a neutral form, a cationic form (e.g., carrying one or more positive charges), or an anionic form (e.g., carrying one or more negative charges), all of which are intended to be included in the scope of the present disclosure. For example, when a compound of the present disclosure is depicted in an anionic form, it should be understood that such depiction also refers to the various neutral forms, cationic forms, and anionic forms of the compound. For another example, when a compound the present disclosure is depicted in an anionic form, it should be understood that such depiction also refers to various salts (e.g., sodium salt) of the anionic form of the compound.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, alkylsulphinyl, sulphonato, sulphamoyl, sulphonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1, l′-isobenzofuran]-yl, 7′H-spiro[cyclohexane-1,5′-furo[3,4-b]pyridin]-yl, 3′H-spiro[cyclohexane-1, l′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the needs rings in the heterocycloalkyl to be non-aromatic (e.g., 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups.

The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more C₅, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; Greene, T. W., Wuts, P. G. M., Protective Groups in Organic Synthesis, 3rd edition, John Wiley & Sons: New York, 1999; R. Larock, Comprehensive Organic Transformations, VCH Publishers (1989); L. Fieser and M. Fieser, Fieser and Fieser's Reagents for Organic Synthesis; John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3rd edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable salt, polymorph or solvate thereof, can or may also be used to prevent a relevant disease, condition, or disorder, or used to identify suitable candidates for such purposes.

As used herein, the term “preventing,” “prevent,” or “protecting against” describes reducing or eliminating the onset of the symptoms or complications of such disease, condition or disorder.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, New York (2000); Coligan et al., Current Protocols in Immunology; John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18th edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulphonic, acetic, ascorbic, benzene sulphonic, benzoic, bicarbonic, carbonic, citric, edetic, ethane disulphonic, 1,2-ethane sulphonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulphonic, maleic, malic, mandelic, methane sulphonic, napsylic, nitric, oxalic, pamoic, pantothenic, phenylacetic, phosphoric, polygalacturonic, propionic, salicylic, stearic, subacetic, succinic, sulphamic, sulphanilic, sulphuric, tannic, tartaric, toluene sulphonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulphonic acid, 2-naphthalenesulphonic acid, 4-toluenesulphonic acid, camphorsulphonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19th edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Exemplary Embodiments

Embodiment 1. A compound of Formula (I′):

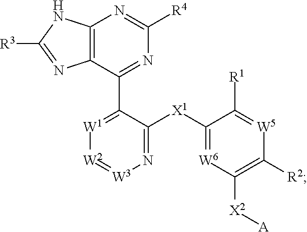

or a pharmaceutically acceptable salt or stereoisomer thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, C₂-C₆ alkynyl, or C₁-C₆ alkoxyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
R² is H, halogen, C₁-C₆ alkyl, C₂-C₆ alkenyl, or C₂-C₆ alkynyl;
R³ is H, halogen, C₁-C₆ alkyl, C₂-C₆ alkenyl, C₂-C₆ alkynyl, or C₁-C₆ alkoxyl, wherein the C₁-C₆ alkoxyl is optionally substituted with one or more OH, NH₂, NH(C₁-C₆ alkyl), or N(C₁-C₆ alkyl)₂;
R⁴ is H, halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is absent, C₂-C₆ alkynyl, —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A};
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH(C₁-C₆ alkyl), or N(C₁-C₆ alkyl)₂ is optionally substituted with one or more R^{A1}, and
each R^{A1} independently is halogen, cyano, OH, NH₂, NH(C₁-C₆ alkyl), N(C₁-C₆ alkyl)₂, C₁-C₆ alkyl, C₂-C₆ alkenyl, or C₂-C₆ alkynyl.

Embodiment 2. The compound of any one of the preceding embodiments, wherein:
W¹ is N or CR^{W1};
R^{W1} is H, halogen, or C₁-C₆ alkyl;
W² is CR^{W2};
R^{W2} is H or halogen;
W³ is N or CR^{W3};
R^{W3} is H or C₁-C₆ alkoxyl;
X¹ is NR^{X1};
R^{X1} is H;
R¹ is C₁-C₆ alkyl;
R² is H or C₁-C₆ alkyl;
R³ is H, C₁-C₆ alkyl, or C₁-C₆ alkoxyl, wherein the C₁-C₆ alkoxyl is optionally substituted with one or more OH or N(C₁-C₆ alkyl)₂;
R⁴ is H, halogen, or C₁-C₆ alkyl;
W⁵ is CR^{W5};
R^{W5} is H;
W⁶ is CR^{W6};
R^{W6} is H;
X² is —NR^{X2}C(O)—* or —NR^{X2}C(O)CH₂—*, wherein * denotes attachment to A;
R^{X2} is H;
A is C₃-C₈ cycloalkyl, 3- to 8-membered heterocycloalkyl, or 5- to 9-membered heteroaryl, wherein the C₃-C₈ cycloalkyl, 3- to 8-membered heterocycloalkyl, or 5- to 9-membered heteroaryl is optionally substituted with one or more R^{A};
each R^{A} independently is halogen, C₁-C₆ alkyl, C₁-C₆ alkoxyl, —O-(3- to 8-membered heterocycloalkyl), NH₂, NH(C₁-C₆ alkyl), or N(C₁-C₆ alkyl)₂, wherein the C₁-C₆ alkyl, C₁-C₆ alkoxyl, or —O-(3- to 8-membered heterocycloalkyl) is optionally substituted with one or more R^{A1}; and
each R^{A1} independently is halogen, cyano, OH, N(C₁-C₆ alkyl)₂, or C₁-C₆ alkyl.

Embodiment 3. A compound of Formula (I):

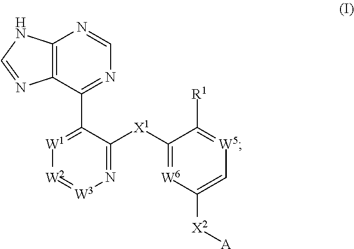

or a pharmaceutically acceptable salt or stereoisomer thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, or C₂-C₆ alkynyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is absent, C₂-C₆ alkynyl, —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}; and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

Embodiment 4. The compound of any one of the preceding embodiments, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, or C₂-C₆ alkynyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}, and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

Embodiment 5. The compound of any one of the preceding embodiments, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, or C₂-C₆ alkynyl;
X¹ is NR^{X1};
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}, and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

Embodiment 6. The compound of any one of the preceding embodiments, wherein:
W¹ is CR^{W1};
R^{W1} is H, halogen, C₁-C₆ alkyl, C₂-C₆ alkenyl, or C₂-C₆ alkynyl;
W² is CR^{W2};
R^{W2} is H, halogen, C₁-C₆ alkyl, C₂-C₆ alkenyl, or C₂-C₆ alkynyl;
W³ is CR^{W3};
R^{W3} is H, halogen, C₁-C₆ alkyl, C₂-C₆ alkenyl, or C₂-C₆ alkynyl;
X¹ is NR^{X1};
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is —NR^{X2}C(O)—*, —C(O)NR^{X2}—*, —NR^{X2}C(O)CH₂—*, or —C(O)NR^{X2}CH₂—*, wherein* denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}, and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

Embodiment 7. The compound of any one of the preceding embodiments, wherein W¹ is CR^{W1}, W² is CR^{W2}, and W³ is CR^{W3}.

Embodiment 8. The compound of any one of the preceding embodiments, wherein R^{W1} is H.

Embodiment 9. The compound of any one of the preceding embodiments, wherein R^{W1} is halogen.

Embodiment 10. The compound of any one of the preceding embodiments, wherein R^{W1} is C₁-C₆ alkyl.

Embodiment 11. The compound of any one of the preceding embodiments, wherein R^{W2} is H.

Embodiment 12. The compound of any one of the preceding embodiments, wherein R^{W2} is halogen.

Embodiment 13. The compound of any one of the preceding embodiments, wherein R^{W3} is H.

Embodiment 14. The compound of any one of the preceding embodiments, wherein R^{W3} is C₁-C₆ alkoxyl.

Embodiment 15. The compound of any one of the preceding embodiments, wherein W¹; W², and W³ each are CH.

Embodiment 16. The compound of any one of the preceding embodiments, wherein W¹ is N.

Embodiment 17. The compound of any one of the preceding embodiments, wherein W¹ is N, W² is CR^{W2}, and W³ is CR^{W3}.

Embodiment 18. The compound of any one of the preceding embodiments, wherein W³ is N.

Embodiment 19. The compound of any one of the preceding embodiments, wherein W¹ is CR^{W1}, W² is CR^{W2}, and W³ is N.

Embodiment 20. The compound of any one of the preceding embodiments, wherein X¹ is NR^{X1}.

Embodiment 21. The compound of any one of the preceding embodiments, wherein X¹ is NH.

Embodiment 22. The compound of any one of the preceding embodiments, wherein X¹ is O or S.

Embodiment 23. The compound of any one of the preceding embodiments, wherein R¹ is halogen.

Embodiment 24. The compound of any one of the preceding embodiments, wherein R¹ is C₁-C₆ alkyl.

Embodiment 25. The compound of any one of the preceding embodiments, wherein R¹ is CH₃.

Embodiment 26. The compound of any one of the preceding embodiments, wherein R² is H.

Embodiment 27. The compound of any one of the preceding embodiments, wherein R² is C₁-C₆ alkyl.

Embodiment 28. The compound of any one of the preceding embodiments, wherein R² is CH₃.

Embodiment 29. The compound of any one of the preceding embodiments, wherein R³ is H.

Embodiment 30. The compound of any one of the preceding embodiments, wherein R³ is C₁-C₆ alkyl.

Embodiment 31. The compound of any one of the preceding embodiments, wherein R³ is CH₃.

Embodiment 32. The compound of any one of the preceding embodiments, wherein R³ is C₁-C₆ alkoxyl.

Embodiment 33. The compound of any one of the preceding embodiments, wherein R³ is —OCH₃.

Embodiment 34. The compound of any one of the preceding embodiments, wherein R³ is C₁-C₆ alkoxyl, optionally substituted with one or more OH, NH₂, NH(C₁-C₆ alkyl), or N(C₁-C₆ alkyl)₂.

Embodiment 35. The compound of any one of the preceding embodiments, wherein R⁴ is H.

Embodiment 36. The compound of any one of the preceding embodiments, wherein R⁴ is halogen.

Embodiment 37. The compound of any one of the preceding embodiments, wherein R⁴ is C₁-C₆ alkyl.

Embodiment 38. The compound of any one of the preceding embodiments, wherein R⁴ is CH₃.

Embodiment 39. The compound of any one of the preceding embodiments, wherein W⁵ is N.

Embodiment 40. The compound of any one of the preceding embodiments, wherein W⁵ is CR^{W5}.

Embodiment 41. The compound of any one of the preceding embodiments, wherein R^{W5} is H.

Embodiment 42. The compound of any one of the preceding embodiments, wherein R^{W5} is halogen.

Embodiment 43. The compound of any one of the preceding embodiments, wherein R^{W5} is C₁-C₆ alkyl.

Embodiment 44. The compound of any one of the preceding embodiments, wherein W⁶ is N.

Embodiment 45. The compound of any one of the preceding embodiments, wherein W⁶ is CR^{W6}.

Embodiment 46. The compound of any one of the preceding embodiments, wherein R^{W6}

Embodiment 47. The compound of any one of the preceding embodiments, wherein R^{W6} is halogen.

Embodiment 48. The compound of any one of the preceding embodiments, wherein R^{W6} is C₁-C₆ alkyl.

Embodiment 49. The compound of any one of the preceding embodiments, wherein X² is absent.

Embodiment 50. The compound of any one of the preceding embodiments, wherein X² is C₂-C₆ alkynyl.

Embodiment 51. The compound of any one of the preceding embodiments, wherein X² is —NR^{X2}C(O)—*, —C(O)NR^{X2}—*, —NR^{X2}C(O)CH₂—*, or —C(O)NR^{X2}CH₂—*, wherein * denotes attachment to A.

Embodiment 52. The compound of any one of the preceding embodiments, wherein X² is —NHC(O)—* or —C(O)NH—*.

Embodiment 53. The compound of any one of the preceding embodiments, wherein X² is —NR^{X2}C(O)CH₂—* or —C(O)NR^{X2}CH₂—*.

Embodiment 54. The compound of any one of the preceding embodiments, wherein X² is —NHC(O)CH₂—* or —C(O)NHCH₂—*.

Embodiment 55. The compound of any one of the preceding embodiments, wherein A is C₃-C₈ cycloalkyl optionally substituted with one or more R^{A}.

Embodiment 56. The compound of any one of the preceding embodiments, wherein A is 3- to 8-membered heterocycloalkyl optionally substituted with one or more R^{A}.

Embodiment 57. The compound of any one of the preceding embodiments, wherein A is C₆-C₁₀ aryl optionally substituted with one or more R^{A}.

Embodiment 58. The compound of any one of the preceding embodiments, wherein A is phenyl optionally substituted with one or more R^{A}.

Embodiment 59. The compound of any one of the preceding embodiments, wherein A is

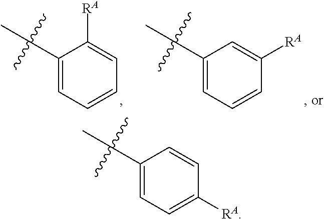

Embodiment 60. The compound of any one of the preceding embodiments, wherein A is

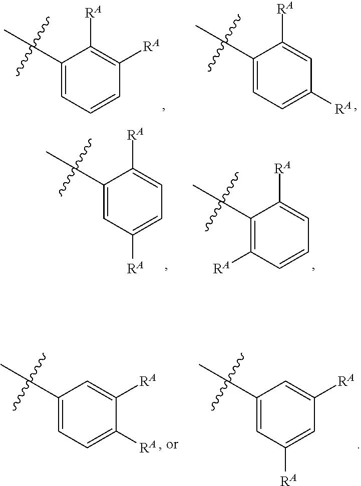

Embodiment 61. The compound of any one of the preceding embodiments, wherein A is 5- to 9-membered heteroaryl optionally substituted with one or more R^{A}.

Embodiment 62. The compound of any one of the preceding embodiments, wherein A is pyridinyl optionally substituted with one or more R^{A}.

Embodiment 63. The compound of any one of the preceding embodiments, wherein A is

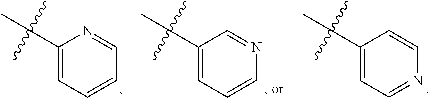 [SMILES: *c1ccccn1 ; *c1cccnc1 ; *c1ccncc1]

Embodiment 64. The compound of any one of the preceding embodiments, wherein A is

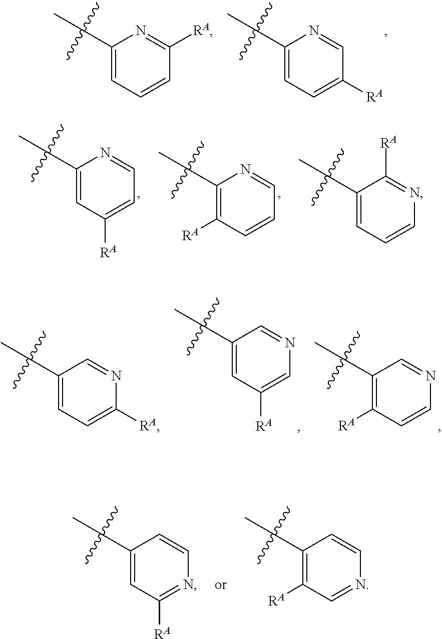

Embodiment 65. The compound of any one of the preceding embodiments, wherein each R^{A} independently is halogen.

Embodiment 66. The compound of any one of the preceding embodiments, wherein each R^{A} independently is F.

Embodiment 67. The compound of any one of the preceding embodiments, wherein each R^{A} independently is Cl.

Embodiment 68. The compound of any one of the preceding embodiments, wherein each R^{A} independently is cyano.

Embodiment 69. The compound of any one of the preceding embodiments, wherein each R^{A} independently is C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

Embodiment 70. The compound of any one of the preceding embodiments, wherein each R^{A} independently is C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH(C₁-C₆ alkyl), or N(C₁-C₆ alkyl)₂ is optionally substituted with one or more R^{A1}.

Embodiment 71. The compound of any one of the preceding embodiments, wherein each R^{A} independently is C₁-C₆ alkyl, C₂-C₆ alkenyl, or C₂-C₆ alkynyl, wherein the C₁-C₆ alkyl, C₂-C₆ alkenyl, C₂-C₆ alkynyl, or C₁-C₆ alkoxyl is optionally substituted with one or more halogen or cyano.

Embodiment 72. The compound of any one of the preceding embodiments, wherein each R^{A} independently is C₁-C₆ alkyl, C₂-C₆ alkenyl, or C₂-C₆ alkynyl, wherein the C₁-C₆ alkyl, C₂-C₆ alkenyl, C₂-C₆ alkynyl, or C₁-C₆ alkoxyl is optionally substituted with one or more R^{A1}.

Embodiment 73. The compound of any one of the preceding embodiments, wherein each R^{A} independently is C₁-C₆ alkyl optionally substituted with one or more R^{A1}.

Embodiment 74. The compound of any one of the preceding embodiments, wherein each R^{A} independently is C₁-C₆ alkoxyl optionally substituted with one or more halogen or cyano.

Embodiment 75. The compound of any one of the preceding embodiments, wherein each R^{A} independently is C₁-C₆ alkoxyl optionally substituted with one or more R^{A1}.

Embodiment 76. The compound of any one of the preceding embodiments, wherein each R^{A} independently is C₃-C₈ cycloalkyl optionally substituted with one or more halogen or cyano.

Embodiment 77. The compound of any one of the preceding embodiments, wherein each R^{A} independently is 3- to 8-membered heterocycloalkyl optionally substituted with one or more halogen or cyano.

Embodiment 78. The compound of any one of the preceding embodiments, wherein each R^{A} independently is —O-(3- to 8-membered heterocycloalkyl) optionally substituted with one or more R^{A1}.

Embodiment 79. The compound of any one of the preceding embodiments, wherein each R^{A} independently is NH₂, NH(C₁-C₆ alkyl), or N(C₁-C₆ alkyl)₂, wherein the NH(C₁-C₆ alkyl) or N(C₁-C₆ alkyl)₂ is optionally substituted with one or more halogen or cyano.

Embodiment 80. The compound of any one of the preceding embodiments, wherein each R^{A} independently is NH₂, NH(C₁-C₆ alkyl), or N(C₁-C₆ alkyl)₂, wherein the NH(C₁-C₆ alkyl) or N(C₁-C₆ alkyl)₂ is optionally substituted with one or more R^{A1}.

Embodiment 81. The compound of any one of the preceding embodiments, wherein each R^{A1} independently is halogen, cyano, OH, NH₂, NH(C₁-C₆ alkyl), N(C₁-C₆ alkyl)₂, C₁-C₆ alkyl, C₂-C₆ alkenyl, or C₂-C₆ alkynyl.

Embodiment 82. The compound of any one of the preceding embodiments, wherein each R^{A1} independently is halogen.

Embodiment 83. The compound of any one of the preceding embodiments, wherein each R^{A1} independently is cyano.

Embodiment 84. The compound of any one of the preceding embodiments, wherein each R^{A1} independently is OH.

Embodiment 85. The compound of any one of the preceding embodiments, wherein each R^{A1} independently is N(C₁-C₆ alkyl)₂.

Embodiment 86. The compound of any one of the preceding embodiments, wherein each R^{A1} independently is C₁-C₆ alkyl.

Embodiment 87. The compound of any one of the preceding embodiments, wherein when W¹, W², W³, W⁵, and W⁶ each are CH; X¹ is NH; X² is —NHC(O)—* or —C(O)NH—*; and R¹ is F, Cl, or CH₃; then A is not

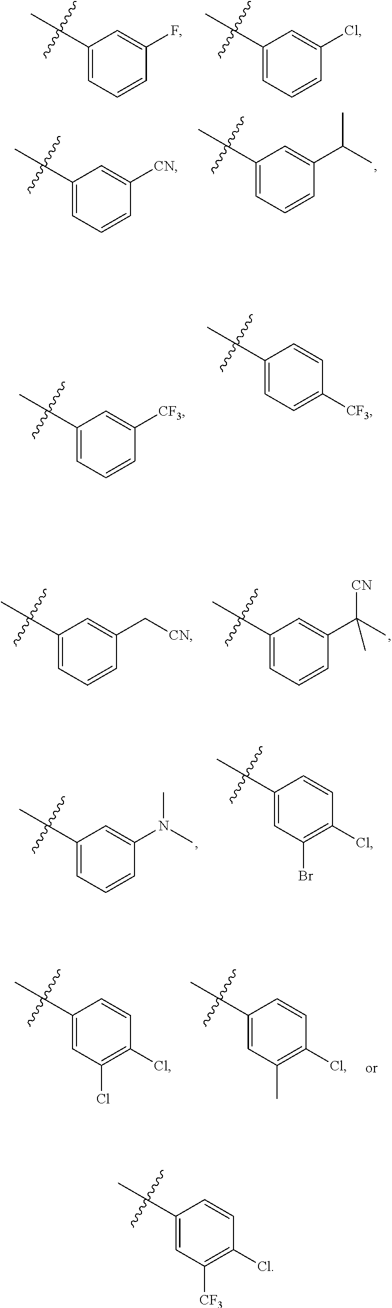

Embodiment 88. The compound of any one of the preceding embodiments, wherein when W¹, W², W³, W⁵, and W⁶ each are CH; X¹ is NH; X² is —NHC(O)—* or —C(O)NH—*; and R¹ is F, C₁, or CH₃;
then A is not phenyl substituted with one or more F, Cl, Br, CN, CH₃, CH(CH₃)₂, CF₃, CH₂CN, C(CH₃)₂CN, or N(CH₃)₂.

Embodiment 89. The compound of any one of the preceding embodiments, wherein when W¹, W², W³, W⁵, and W⁶ each are CH; X¹ is NH; X² is —NHC(O)—* or —C(O)NH—*; and R¹ is halogen or C₁-C₆ alkyl;
then A is not C₆-C₁₀ aryl substituted with one or more halogen, cyano, C₁-C₆ alkyl optionally substituted with one or more halogen or cyano, or N(C₁-C₆ alkyl)₂.

Embodiment 90. The compound of any one of the preceding embodiments, wherein when W¹, W², W³, W⁵, and W⁶ each are CH; X¹ is NH; X² is —NHC(O)—* or —C(O)NH—*; and R¹ is halogen or C₁-C₆ alkyl;
then A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}; and each R^{A} independently is C₂-C₆ alkenyl, C₂-C₆ alkynyl, C₁-C₆ alkoxyl, C₃-C₈ cycloalkyl, or 3- to 8-membered heterocycloalkyl, wherein the C₂-C₆ alkenyl, C₂-C₆ alkynyl, C₁-C₆ alkoxyl, C₃-C₈ cycloalkyl, or 3- to 8-membered heterocycloalkyl is optionally substituted with one or more halogen or cyano.

Embodiment 91. The compound of any one of the preceding embodiments, wherein when R¹ is F, Cl, or CH₃; then A is not

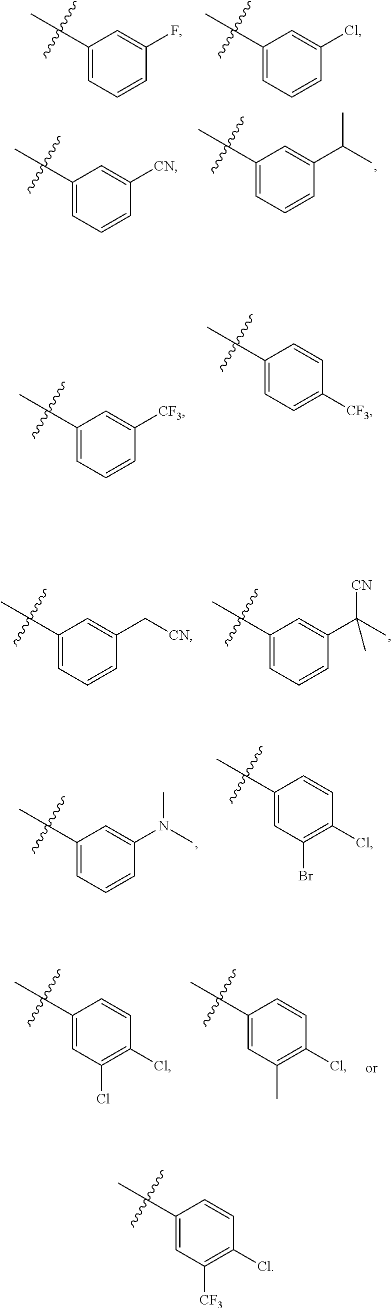

Embodiment 92. The compound of any one of the preceding embodiments, wherein when R¹ is F, Cl, or CH₃; then A is not phenyl substituted with one or more F, Cl, Br, CN, CH₃; CH(CH₃)₂, CF₃, CH₂CN, C(CH₃)₂CN, or N(CH₃)₂.

Embodiment 93. The compound of any one of the preceding embodiments, wherein when R¹ is halogen or C₁-C₆ alkyl; then A is not C₆-C₁₀ aryl substituted with one or more halogen, cyano, C₁-C₆ alkyl optionally substituted with one or more halogen or cyano, or N(C₁-C₆ alkyl)₂.

Embodiment 94. The compound of any one of the preceding embodiments, wherein when R¹ is halogen or C₁-C₆ alkyl; then A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}; and each R^{A} independently is C₂-C₆ alkenyl, C₂-C₆ alkynyl, C₁-C₆ alkoxyl, C₃-C₈ cycloalkyl, or 3- to 8-membered heterocycloalkyl, wherein the C₂-C₆ alkenyl, C₂-C₆ alkynyl, C₁-C₆ alkoxyl, C₃-C₈ cycloalkyl, or 3- to 8-membered heterocycloalkyl is optionally substituted with one or more halogen or cyano.

Embodiment 95. The compound of any one of the preceding embodiments, being of Formula (II):

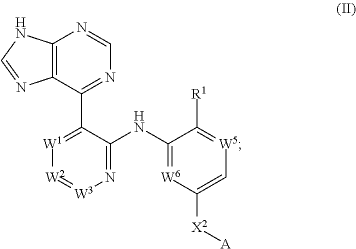

or a pharmaceutically acceptable salt or stereoisomer thereof.

Embodiment 96. The compound of any one of the preceding embodiments, being of Formula (III-a), (III-b), (III-c), (III-d), (III-e), or (III-f):

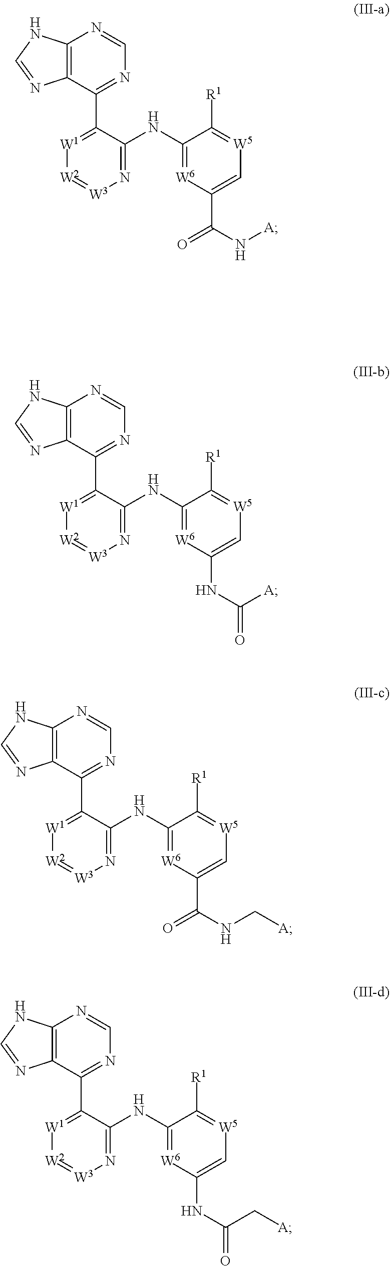

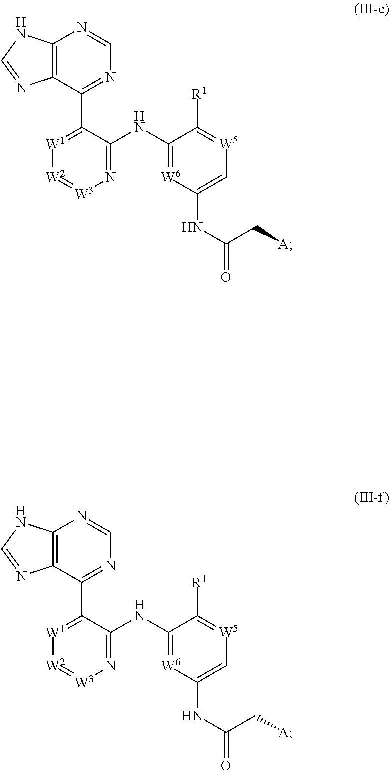

or a pharmaceutically acceptable salt or stereoisomer thereof.

Embodiment 97. The compound of any one of the preceding embodiments, being of Formula (IV-a) or (IV-b):

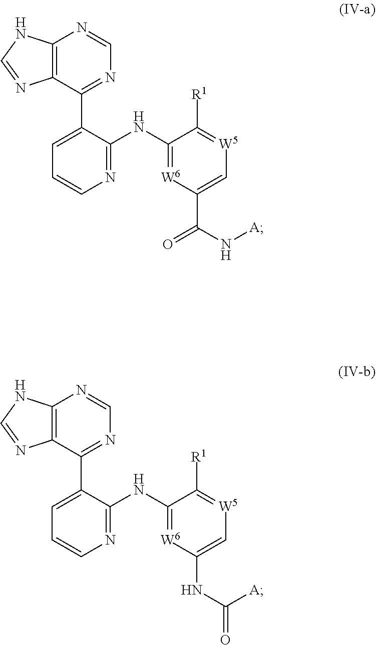

or a pharmaceutically acceptable salt or stereoisomer thereof.

Embodiment 98. The compound of any one of the preceding embodiments, being selected from the compounds described in Tables I, IA and II, or a pharmaceutically acceptable salt or stereoisomer thereof.

Embodiment 99. The compound of any one of the preceding embodiments, being selected from the compounds described in Table I and Table IA, or a pharmaceutically acceptable salt or stereoisomer thereof.

Embodiment 100. The compound of any one of the preceding embodiments, being selected from the compounds described in Table I, or a pharmaceutically acceptable salt or stereoisomer thereof.

Embodiment 101. The compound of any one of the preceding embodiments, being selected from the compounds described in Table IA, or a pharmaceutically acceptable salt or stereoisomer thereof.

Embodiment 102. The compound of any one of the preceding embodiments, being selected from the compounds described in Table II, or a pharmaceutically acceptable salt or stereoisomer thereof.

Embodiment 103. An isotopic derivative of the compound of any one of the preceding embodiments.

Embodiment 104. A method of preparing the compound of any one of the preceding embodiments.

Embodiment 105. A pharmaceutical composition comprising the compound of any one of the preceding embodiments and one or more pharmaceutically acceptable carriers or excipients.

Embodiment 106. A method of treating or preventing cancer in a subject, the method comprising administering to the subject a compound of any one of the preceding embodiments.

Embodiment 107. A compound of any one of the preceding embodiments for treating or preventing cancer in a subject.

Embodiment 108. Use of the compound of any one of the preceding embodiments in the manufacture of a medicament for treating or preventing cancer in a subject.

Embodiment 109. Use of the compound of any one of the preceding embodiments for treating or preventing cancer in a subject.

Embodiment 110. The method, compound, or use of any one of the preceding embodiments, wherein the subject is a human.

Embodiment 111. The method, compound, or use of any one of the preceding embodiments, wherein the cancer is characterized by at least one oncogenic mutation in the BRAF gene.

Embodiment 112. The method, compound, or use of any one of the preceding embodiments, wherein the cancer is characterized by at least one oncogenic variant of B-Raf.

Embodiment 113. The method, compound, or use of any one of the preceding embodiments, wherein the subject has at least one oncogenic mutation in the BRAF gene.

Embodiment 114. The method, compound, or use of any one of the preceding embodiments, wherein the subject has at least one tumor and/or cancerous cell that expresses an oncogenic variant of B-Raf.

Embodiment 115. The method, compound, or use of any one of the preceding embodiments, wherein the oncogenic mutation is a class II mutation. Accordingly, in some embodiments, the oncogenic variant of B-Raf comprises a class II mutation.

Embodiment 116. The method, compound, or use of any one of the preceding embodiments, wherein the oncogenic mutation is a class III mutation.

Embodiment 117. The method, compound, or use of any one of the preceding embodiments, wherein the oncogenic variant of B-Raf can be any of the B-Raf variants put forth in Table 1b.

Embodiment 118. The method, compound, or use of any one of the preceding embodiments, wherein the cancer is a hematological cancer.

Embodiment 119. The method, compound, or use of any one of the preceding embodiments, wherein the cancer is a solid cancer.

Embodiment 120. The method, compound, or use of any one of the preceding embodiments, wherein the cancer is melanoma, breast cancer, head and neck cancer, esophagogastric cancer, stomach and small intestine cancer, lung cancer, mesothelioma, hepatobiliary cancer, pancreatic cancer, kidney cancer, colorectal cancer, endometrial cancer, cervical cancer, ovarian cancer, bladder cancer, prostate cancer, soft tissue sarcoma, CNS and brain cancer, or thyroid cancer.

Embodiment 121. The method, compound, or use of any one of the preceding embodiments, wherein the cancer is non-small cell lung cancer (NSCLC), colorectal cancer, melanoma, thyroid cancer, histiocytosis, small bowel cancer, gastrointestinal neuroendocrine cancer, carcinoma of unknown primary, non-melanoma skin cancer, prostate cancer, gastric cancer, non-Hodgkin's lymphoma, papillary thyroid carcinoma or glioblastoma.

### EXAMPLES

For exemplary purpose, salts of the compounds of Formula (I′) and (I) are synthesized and tested in the examples. It is understood that neutral compounds of Formula (I′) and (I) may be similarly synthesized and tested using the exemplary procedures described in the examples. Further, it is understood that the salts (e.g., sodium salt) of the compounds of Formula (I′) and (I) may be converted to the corresponding neutral compounds using routine techniques in the art (e.g., pH adjustment and, optionally, extraction (e.g., into an aqueous phase)).

Compounds of Formula (I′) and (I) can be prepared using the methods detailed herein. Those skilled in the art may be able to envisage alternative synthetic routes, using a variety of starting materials and reagents to prepare the disclosed compounds of Formula (I′) and (I) and to make further modifications. For exemplary purpose, salts of some of the compounds of Formula (I′) and (I) are synthesized and tested in the examples. It is understood that neutral compounds of Formula (I′) and (I) may be similarly synthesized and tested using the exemplary procedures described in the examples. Further, it is understood that the salts (e.g., hydrochloride salt) of the compounds of Formula (I′) and (I) may be converted to the corresponding neutral compounds using routine techniques in the art (e.g., pH adjustment and, optionally, extraction (e.g., into an aqueous phase)).

### Abbreviations:

¹H NMR Proton nuclear magnetic resonance spectroscopy
ACN Acetonitrile
aq. Aqueous
BPO Benzoyl peroxide
BuLi Butyllithium
CbzCl Benzyl chloroformate
CDCl₃ Deuterated chloroform
DAST Diethylaminosulphur trifluoride
DCE Dichloroethane
DCM Dichloromethane
DHP 3,4-Dihydro-2H-pyran
DIEA N,N-Diisopropylethylamine
DIPA Diisopropylamine
DIPEA N,N-Diisopropylethylamine
DMA Dimethylacetamide
DMAP 4-Dimethylaminopyridine
DME 1,2-Dimethoxyethane
DMF Dimethylformamide
DMPU 1,3-Dimethyl-3,4,5,6-tetrahydro-2(1H)-pyrimidinone
DMSO Dimethyl sulfoxide
DMSO-d₆ Hexadeuterodimethylsulfoxide
eq. Equivalents
Et3N Triethylamine
Et3 SiH Triethylsilane
EtOAc Ethyl acetate
EtOH Ethanol
h Hour(s)
HATU 1-[bis(dimethylamino)methylene]-1H-1,2,3-triazolo[4,5-b]pyridinium 3-oxid
HPLC hexafluorophosphate
High performance liquid chromatography
LAH Lithium aluminum hydride
LC-MS Liquid chromatography-mass spectrometry
LDA Lithium diisopropylamide
LiHMDS Lithium hexamethyldisilazide
mCPBA 3-Chloroperbenzoic acid
MeCN Acetonitrile
MeOH Methanol
min Minute(s)
MsCl Methanesulfonyl chloride
NBS N-Bromosuccinimide
NCS N-Chlorosuccinimide
NIS N-Iodosuccinimide
NMP 1-Methyl-2-pyrrolidinone
prep-HPLC preparative high performance liquid chromatography
Py Pyridine
rt room temperature
SEMCl 2-(trimethylsilyl)ethoxymethyl chloride
TBAF Tetrabutylammonium fluoride
TBTU 2-(1H-benzotriazole-1-yl)-1,1,3,3-tetramethylaminium tetrafluoroborate
TEA Triethylamine
TFA Trifluoroacetic acid
TFAA Trifluoroacetic anhydride
THF Tetrahydrofuran
TLC Thin layer chromatography
TMP 2,2,6,6-Tetramethylpiperidine
TMSCF3 Trimethyl(trifluoromethyl)silane
TMSCl Chlorotrimethylsilane
TMSCN Trimethylsilyl cyanide
TsCl Tosyl chloride
TsOH 4-Methylbenzene sulfonic acid
UHP Urea hydrogen peroxide
Y Yield

### Example 1. Synthesis of 3-(1-Cyano-1-methyl-ethyl)-N—[4-methyl-3-[[3-(9H-purin-6-yl)-2-pyridyl]amino]phenyl]benzamide (Compound II-1)

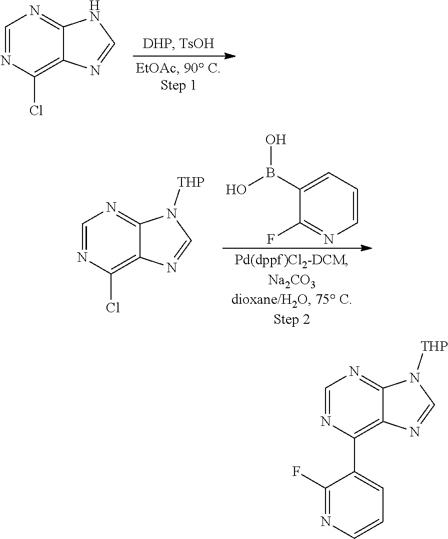

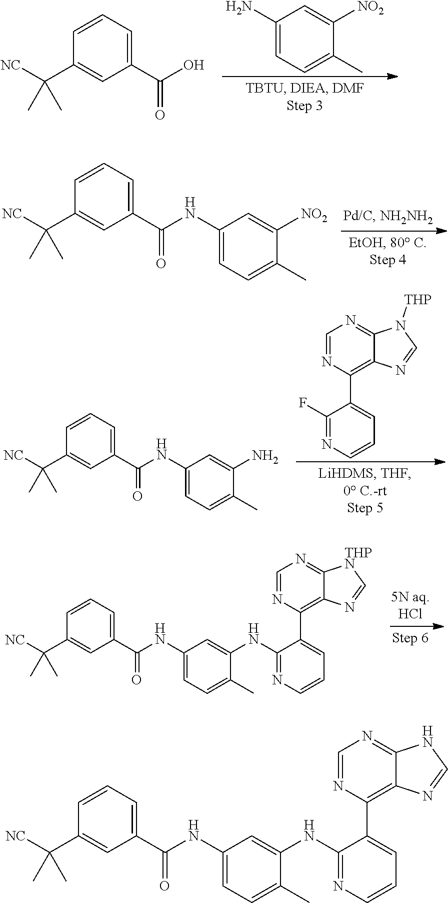

Step 1. A suspension of 6-chloro-9H-purine (40 g, 259.7 mmol) and 4-methylbenzene sulfonic acid (896 mg, 5.2 mmol) in ethyl acetate (300 mL) was treated with 3,4-dihydro-2H-pyran (65.2 g, 779 mmol). The mixture was heated at 90° C. for 16 h. The mixture was concentrated in vacuo. The pale yellow residue was dissolved in dichloromethane and purified by flash chromatography (petroleum ether/ethyl acetate=100/38) to give 6-chloro-9-(tetrahydro-2H-pyran-2-yl)-9H-purine (46.6 g, 195.8 mmol, 75%) as a white solid. MS (ESI) m/z 239.1 [M+H]⁺

Step 2. A solution of 6-chloro-9-(tetrahydro-2H-pyran-2-yl)-9H-purine (46.6 g, 195.8 mmol), 2-fluoropyridin-3-ylboronic acid (41.4 g, 293.7 mmol), 1,1′-bis(diphenylphosphino) ferrocene-palladium(II) dichloride dichloromethane complex (16 g, 19.6 mmol) and sodium carbonate (41.5 g, 391.6 mmol) in water (200 mL) and dioxane (1000 mL) was stirred at 75° C. for 3 hours under argon. The reaction mixture was cooled. The organic layer was concentrated and purified by flash chromatography (Biotage, 40 g silica gel, petroleum ether in ethyl acetate from 30% to 100%) to give 6-(2-fluoropyridin-3-yl)-9-(tetrahydro-2H-pyran-2-yl)-9H-purine (33.4 g, 111.6 mmol, 57%) as yellow solid. MS (ESI) m/z 300.1 [M+H]⁺

Step 3. To a mixture of 3-(1-cyano-1-methyl-ethyl) benzoic acid (500 mg, 2.64 mmol), 2-(1H-benzotriazole-1-yl)-1,1,3,3-tetramethylaminium tetrafluoroborate (TBTU) (1.02 g, 3.17 mmol), N,N-diisopropylethylamine (1.02 g, 7.93 mmol) in dimethylformamide (2.0 mL) was added 4-methyl-3-nitro-aniline (402 mg, 2.64 mmol). The reaction mixture was stirred at 20° C. for 3 hours. On completion, the mixture was poured into 10 mL water and extracted with ethyl acetate (10 mL×3). The combined organic layers were washed with water, brine, dried over sodium sulfate and concentrated in vacuo to give 3-(1-cyano-1-methyl-ethyl)-N-(4-methyl-3-nitro-phenyl) benzamide (800 mg, 2.47 mmol, 93%) as a yellow oil. ¹H NMR (400 MHZ, CDCl₃) δ8.58 (s, 1H), 8.31 (d, J=2.0 Hz, 1H), 8.03 (t, J=2.0 Hz, 1H), 7.97 (dd, J=2.4, 8.4 Hz, 1H), 7.86 (d, J=7.6 Hz, 1H), 7.74-7.70 (m, 1H), 7.57-7.50 (m, 1H), 7.35 (d, J=8.4 Hz, 1H), 2.59 (s, 3H), 1.79 (s, 6H).

Step 4. A mixture of 3-(1-cyano-1-methyl-ethyl)-N-(4-methyl-3-nitro-phenyl)benzamide (600 mg, 1.86 mmol) and palladium(0) on activated carbon (50 mg, 10% purity) in hydrazine hydrate (2.06 g, 34.9 mmol, 2.0 mL, 85% purity) and ethanol (6.0 mL) was stirred at 80° C. for 2 hours. On completion, the palladium catalyst was removed by filtration and the filtrate was concentrated in vacuo. The residue was purified by flash chromatography (petroleum ether/ethyl acetate=10/1 to 1/1) to give N-(3-amino-4-methyl-phenyl)-3-(1-cyano-1-methyl-ethyl)benzamide (500 mg, 1.70 mmol, 91%) as a colorless oil. ¹H NMR (400 MHZ, DMSO-d₆) δ 9.99 (s, 1H), 8.01 (t, J=1.6 Hz, 1H), 7.90 (d, J=7.6 Hz, 1H), 7.75-7.69 (m, 1H), 7.61-7.53 (m, 1H), 7.08 (d, J=2.0 Hz, 1H), 6.92-6.85 (m, 1H), 6.84-6.79 (m, 1H), 4.87 (s, 2H), 2.03 (s, 3H), 1.75 (s, 6H). MS (ESI) m/z 294.1 [M+H]⁺

Step 5. A solution of N-(3-amino-4-methyl-phenyl)-3-(1-cyano-1-methyl-ethyl)benzamide (400 mg, 1.36 mmol) and 6-(2-fluoropyridin-3-yl)-9-(tetrahydro-2H-pyran-2-yl)-9H-purine (408 mg, 1.36 mmol) in tetrahydrofuran (10 mL) was cooled to 0° C., then lithium hexamethyldisilazide (1.0 M in tetrahydrofuran, 4.09 mL) was added dropwise. After addition, the mixture was warmed to 20° C. with stirring for 2 hours. On completion, the mixture was poured into 20 mL saturated ammonium chloride aqueous solution and extracted with ethyl acetate (20 mL×3). The combined organic layers were washed with water, brine and concentrated in vacuo. The residue was triturated with 10 mL methanol and then filtered. The solid was collected to give 3-(1-cyano-1-methyl-ethyl)-N—[4-methyl-3-[[3-(9-tetrahydropyran-2-ylpurin-6-yl)-2-pyridyl]amino]phenyl]benzamide (500 mg, 0.873 mmol, 64%) as a yellow solid. ¹H NMR (400 MHz, DMSO-d₆) δ 12.16 (s, 1H), 10.30 (s, 1H), 9.73 (d, J=8.0 Hz, 1H), 9.14 (s, 1H), 8.96 (s, 1H), 8.62 (s, 1H), 8.35 (d, J=4.0 Hz, 1H), 8.06 (s, 1H), 7.95 (d, J=7.6 Hz, 1H), 7.73 (d, J=8.0 Hz, 1H), 7.62-7.54 (m, 1H), 7.45 (d, J=7.6 Hz, 1H), 7.02 (dd, J=4.8, 7.6 Hz, 1H), 5.88 (d, J=10.4 Hz, 1H), 4.05 (d, J=12.0 Hz, 1H), 3.84-3.69 (m, 1H), 2.42 (s, 3H), 2.38-2.30 (m, 1H), 2.09-1.98 (m, 2H), 1.82-1.76 (m, 1H), 1.75 (s, 6H), 1.67-1.55 (m, 2H).

Step 6. To a mixture of 3-(1-cyano-1-methyl-ethyl)-N—[4-methyl-3-[[3-(9-tetrahydropyran-2-ylpurin-6-yl)-2-pyridyl]amino]phenyl]benzamide (150 mg, 0.261 mmol) in tetrahydrofuran (10 mL) was added aqueous hydrochloric acid solution (5 N, 5.0 mL) and the mixture was stirred at 20° C. for 16 h. On completion, the mixture was concentrated. The residue was triturated with a solvent mixture of ethanol and water (2:1, 10 mL), filtered, and the solid was lyophilized to give 3-(1-cyano-1-methyl-ethyl)-N—[4-methyl-3-[[3-(9H-purin-6-yl)-2-pyridyl]amino]phenyl]benzamide (113 mg, 0.215 mmol, 82%) as a yellow solid.

¹H NMR (400 MHZ, DMSO-d₆) δ 12.54-12.39 (m, 1H), 10.37 (s, 1H), 9.94-9.84 (m, 1H), 9.16 (s, 1H), 8.81 (s, 1H), 8.63 (s, 1H), 8.39 (d, J=2.8 Hz, 1H), 8.12 (s, 1H), 8.02 (d, J=7.6 Hz, 1H), 7.81 (d, J=7.6 Hz, 1H), 7.70-7.63 (m, 1H), 7.58-7.52 (m, 1H), 7.34 (d, J=8.0 Hz, 1H), 7.14 (dd, J=4.8, 7.6 Hz, 1H), 2.49 (s, 3H), 1.82 (s, 6H). MS (ESI) m/z 489.2 [M+H]⁺

### Example 2. Synthesis of N-(3-(3-(9H-Purin-6-yl)pyridin-2-ylamino)-4-methylphenyl)-5-(trifluoromethyl) nicotinamide (Compound I-1)

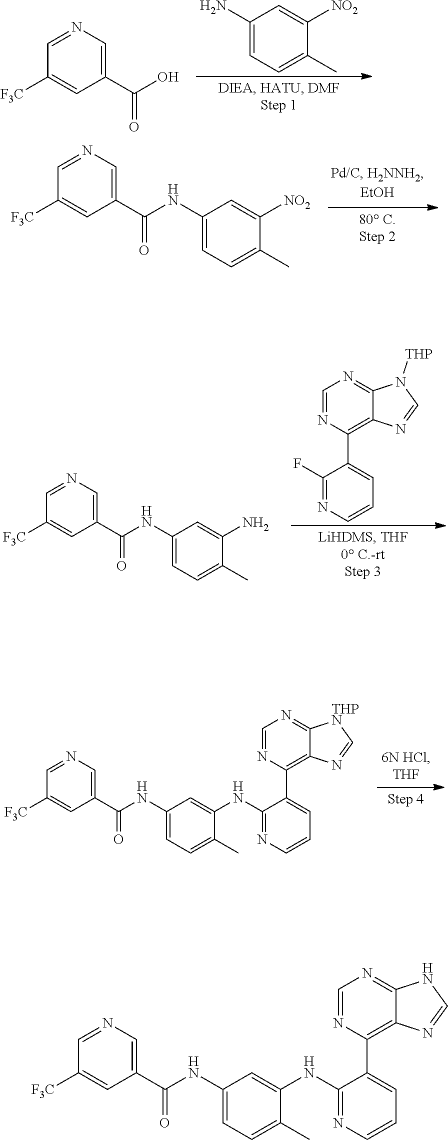

Step 1. To a solution of 5-(trifluoromethyl)nicotinic acid (1.5 g, 7.85 mmol) in N,N-dimethylformamide (15 mL) was added N,N-diisopropylethylamine (3.0 g, 23.6 mmol), 4-methyl-3-nitroaniline (1.2 g, 7.85 mmol) and 1-[bis(dimethylamino)methylene]-1H-1,2,3-triazolo[4,5-b]pyridinium 3-oxid hexafluorophosphate (4.5 g 11.8 mmol) at room temperature. Then the reaction mixture was stirred at room temperature for 1 h. The reaction was diluted with ethyl acetate (100 mL×3), washed with saturated sodium bicarbonate aqueous solution (50 mL) and brine (40 mL), dried over sodium sulfate, filtered, and concentrated. The residue was purified by flash chromatography (petroleum ether/ethyl acetate=4/1) to give N-(4-methyl-3-nitrophenyl)-5-(trifluoromethyl)nicotinamide (2.2 g, 6.8 mmol, 88%) as a white solid. MS (ESI) m/z 326.1[M+H]⁺

Step 2. To a mixture of N-(4-methyl-3-nitrophenyl)-5-(trifluoromethyl)nicotinamide (100 mg, 0.30 mmol) and hydrazine hydrate (2.0 g, 40 mmol) in ethanol (5 mL) was added 10% palladium on carbon (100 mg) at room temperature. The mixture was stirred at 80° C. for 1 h. After cooling to room temperature, the mixture was diluted with ethyl acetate (40 mL) and filtered through celite. The filtrate was concentrated and dried to give N-(3-amino-4-methylphenyl)-5-(trifluoromethyl)nicotinamide (80 mg, 0.27 mmol, 89%) as a white solid. MS (ESI) m/z 296.1 [M+H]⁺

Step 3. Lithium hexamethyldisilazide (1.6 M in tetrahydrofuran, 0.5 mL, 0.8 mmol) was added to a mixture of N-(3-amino-4-methylphenyl)-5-(trifluoromethyl)nicotinamide (80 mg, 0.27 mmol) and 6-(2-fluoropyridin-3-yl)-9-(tetrahydro-2H-pyran-2-yl)-9H-purine (81 mg, 0.27 mmol) in tetrahydrofuran (5 mL) at 0° C. under nitrogen. Then the reaction mixture was stirred at room temperature for 2 h. The reaction mixture was diluted with ethyl acetate (200 mL), washed with saturated sodium bicarbonate aqueous solution (50 mL) and brine (40 mL), then dried over sodium sulfate. The filtrate was concentrated under reduced pressure. The residue was purified by flash chromatography (dichloromethane/methanol=10/1) to give N-(4-methyl-3-(3-(9-(tetrahydro-2H-pyran-2-yl)-9H-purin-6-yl)pyridin-2-ylamino)phenyl)-5-(trifluoromethyl)nicotinamide (150 mg, 0.26 mmol, 96%) as a yellow solid. MS (ESI) m/z 574.7 [M+H]⁺

Step 4. To a mixture of N-(4-methyl-3-(3-(9-(tetrahydro-2H-pyran-2-yl)-9H-purin-6-yl)pyridin-2-yl amino)phenyl)-5-(trifluoromethyl)nicotinamide (150 mg, 0.26 mmol) in tetrahydrofuran (5 mL) was added hydrochloric acid (6 N, 6 mL) at room temperature. Then the reaction mixture was stirred at room temperature for 16 h. The reaction was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-(3-(9H-purin-6-yl)pyridin-2-ylamino)-4-methylphenyl)-5-(trifluoromethyl) nicotinamide (21 mg, 0.04 mmol, 25%) as a yellow solid. ¹H NMR (400 MHZ, DMSO-d6) δ 13.88 (s, 1H), 12.39 (s, 1H), 10.61 (s, 1H), 9.84 (d, J=7.2 Hz, 1H), 9.39 (d, J=1.2 Hz, 1H), 9.18 (d, J=1.2 Hz, 1H), 9.10 (s, 1H), 8.73-8.71 (m, 3H), 8.37 (m, 1H), 7.50 (m, 1H), 7.26 (m, 1H), 7.05 (m, 1H), 2.46 (s, 3H). MS (ESI) m/z 491.1 [M+H]⁺

### Example 3. Synthesis of N-(3-(3-(9H-Purin-6-yl)pyridin-2-ylamino)-4-methylphenyl)-4-(trifluoromethyl)picolinamide (Compound I-2)

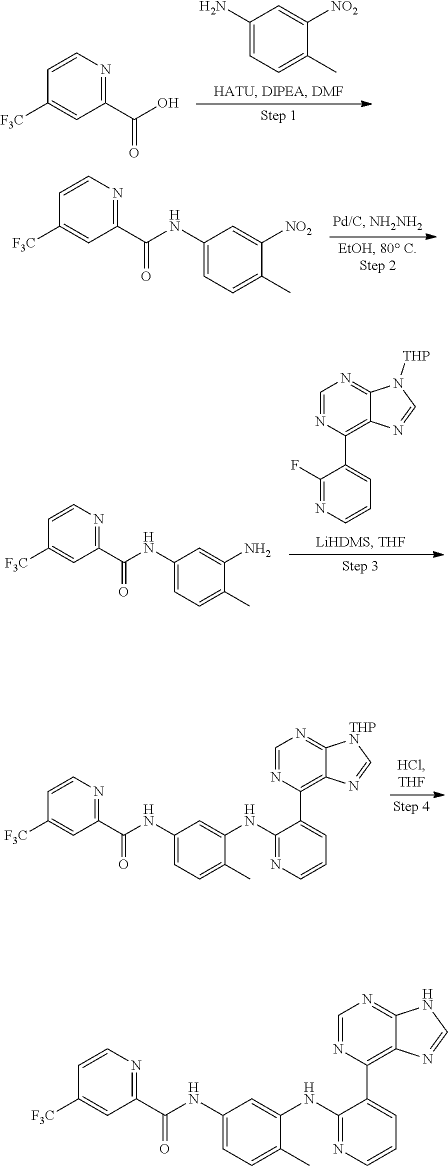

Step 1. A solution of 4-(trifluoromethyl)picolinic acid (1.0 g, 5.23 mmol), 4-methyl-3-nitroaniline (796 mg, 5.23 mmol), 1-[bis(dimethylamino)methylene]-1H-1,2,3-triazolo[4,5-b]pyridinium 3-oxid hexafluorophosphate (2.98 g, 7.85 mmol) and N,N-diisopropylethylamine (2.03 g, 15.7 mmol) in N,N-dimethylformamide (10 mL) was stirred at 20° C. for 1 h. The mixture was diluted with ethyl acetate (150 mL) and washed with water (150 mL). The organic phase was washed with brine (40 mL), dried over sodium sulfate, filtered, and concentrated. The residue was purified by flash chromatography (ethyl acetate/petroleum ether=50/100) to get N-(4-methyl-3-nitrophenyl)-4-(trifluoromethyl)picolinamide (920 mg, 2.83 mmol, 54%) as a yellow solid. ¹H NMR (400 MHZ, Chloroform-d) δ10.05 (s, 1H), 8.84 (d, J=5.0 Hz, 1H), 8.57-8.51 (m, 1H), 8.40 (d, J=2.3 Hz, 1H), 8.01 (dd, J=8.3, 2.4 Hz, 1H), 7.78-7.74 (m, 1H), 7.39 (d, J=8.4 Hz, 1H), 2.61 (s, 3H). MS (ESI) m/z 326.1 [M+H]⁺

Step 2. To a solution of N-(4-methyl-3-nitrophenyl)-4-(trifluoromethyl)picolinamide (1.0 g, 0.31 mmol) and hydrazine hydrate (5 mL) in ethanol (100 mL) was added palladium on activated carbon (10% Pd, 500 mg). The mixture was stirred at 80° C. for 1 h. The reaction mixture was cooled, filtered, and concentrated. The residue was diluted with ethyl acetate (100 mL) and washed with water (100 mL). The organic phase was washed with brine (60 mL), dried over sodium sulfate, filtered, and concentrated to give crude N-(3-amino-4-methylphenyl)-4-(trifluoromethyl)picolinamide (818 mg, 2.77 mmol, 89%) as a yellow solid. MS (ESI) m/z 296.1 [M+H]⁺

Step 3. To a solution of N-(3-amino-4-methylphenyl)-4-(trifluoromethyl)picolinamide (200 mg, 0.68 mmol), 6-(2-fluoropyridin-3-yl)-9-(tetrahydro-2H-pyran-2-yl)-9H-purine (204 mg, 0.68 mmol) in tetrahydrofuran (10 mL) was added lithium hexamethyldisilazide (2.1 mL, 3.4 mmol, 1.6 M in tetrahydrofuran) at 0° C. slowly. The mixture was stirred at 20° C. for 5 h. The reaction was quenched with saturated ammonium chloride solution (20 mL) at 0° C. and extracted with ethyl acetate (50 mL×3). The combined organic layers were washed with brine (50 mL), dried over sodium sulfate and filtered. The filtrate was concentrated under reduced pressure. The crude residue was purified by flash chromatography (petroleum/ethyl acetate=0/100) to give N-(4-methyl-3-(3-(9-(tetrahydro-2H-pyran-2-yl)-9H-purin-6-yl)pyridin-2-ylamino)phenyl)-4-(trifluormethyl)picolinamide (150 mg, 0.26 mmol, 38%) as a yellow solid. MS (ESI) m/z 575.0 [M+H]⁺

Step 4. To a solution of N-(4-methyl-3-(3-(9-(tetrahydro-2H-pyran-2-yl)-9H-purin-6-yl)pyridin-2-ylamino)phenyl)-4-(trifluoromethyl)picolinamide (150 mg, 0.26 mmol) in tetrahydrofuran (21 mL) was added 6 N hydrochloric acid (3.5 mL) at 20° C., and the mixture was stirred at 20° C. for 5 h. The reaction mixture was concentrated and the residue was diluted with water (5 mL). The pH was adjusted to 8 by adding saturated sodium bicarbonate solution. The resulting crystalline was collected by filtration, washed with water (5 mL) and dried to offer N-(3-(3-(9H-purin-6-yl)pyridin-2-ylamino)-4-methyl phenyl)-4-(trifluoromethyl)picolinamide (50.4 mg, 0.1 mmol, 38%) as a yellow solid. ¹H NMR (400 MHz, DMSO-d₆) δ 13.88 (s, 1H), 12.36 (s, 1H), 10.66 (s, 1H), 9.83 (dd, J=7.8, 2.0 Hz, 1H), 9.09 (s, 1H), 9.04 (d, J=5.1 Hz, 1H), 8.84 (d, J=2.2 Hz, 1H), 8.73 (s, 1H), 8.43-8.31 (m, 2H), 8.10 (dd, J=5.3, 1.7 Hz, 1H), 7.55 (dd, J=8.2, 2.2 Hz, 1H), 7.26 (d, J=8.3 Hz, 1H), 7.05 (dd, J=7.9, 4.6 Hz, 1H), 2.45 (s, 3H). MS (ESI) m/z 490.9 [M+H]⁺

### Example 4. Synthesis of N-(3-(3-(9H-purin-6-yl)pyridin-2-ylamino)-4-methylphenyl)-6-(trifluoromethyl) picolinamide (Compound I-3)

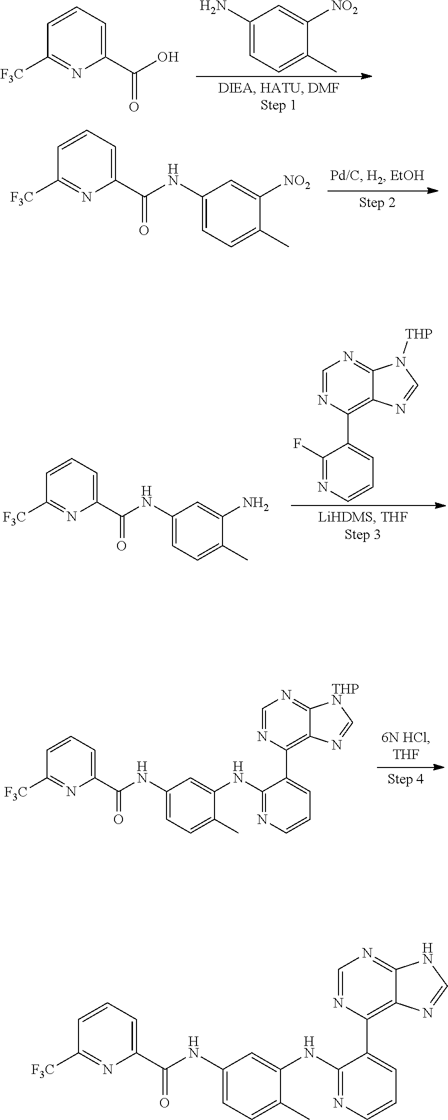

Step 1. To a mixture of 6-(trifluoromethyl)picolinic acid (2.0 g, 10.5 mmol) in N,N-dimethylformamide (15 mL) was added N,N-diisopropylethylamine (4.1 g, 31.4 mmol), 4-methyl-3-nitroaniline (1.6 g, 10.5 mmol) and 1-[bis(dimethylamino)methylene]-1H-1,2,3-triazolo[4,5-b]pyridinium 3-oxid hexafluorophosphate (6.0 g 15.7 mmol) at room temperature. Then the reaction mixture was stirred at room temperature for 1 h. The reaction was diluted with ethyl acetate (200 mL), washed with saturated sodium bicarbonate aqueous solution (50 mL) and brine (40 mL), dried over sodium sulfate, filtered, and concentrated. The residue was purified by flash chromatography (petroleum ether/ethyl acetate=4/1) to give N-(4-methyl-3-nitrophenyl)-6-(trifluoromethyl) picolinamide (3 g, 9.2 mmol, 88%) as a white solid. MS (ESI) m/z 326.1[M+H]⁺

Step 2. To a mixture of N-(4-methyl-3-nitrophenyl)-6-(trifluoromethyl)picolinamide (100 mg, 0.30 mmol) in ethanol (5 mL) was added 10% palladium on carbon (100 mg). The mixture was stirred at room temperature for 0.5 h under a hydrogen atmosphere. The mixture was diluted with ethyl acetate (40 mL) and filtered through celite. The filtrate was concentrated and dried to give N-(3-amino-4-methylphenyl)-3-(trifluoromethyl)benzamide (90 mg, 0.0.31 mmol, 99%) as a white solid. MS (ESI) m/z 296.1 [M+H]⁺

Step 3. Lithium hexamethyldisilazide (1.6 M in tetrahydrofuran, 0.58 mL, 0.93 mmol) was added to a mixture of N-(3-amino-4-methylphenyl)-3-(trifluoromethyl)benzamide (90 mg, 0.31 mmol) and 6-(2-fluoropyridin-3-yl)-9-(tetrahydro-2H-pyran-2-yl)-9H-purine (91 mg, 0.31 mmol) in tetrahydrofuran (5 mL) at 0° C. under nitrogen. Then the reaction mixture was stirred at room temperature for 2 h. The reaction was diluted with ethyl acetate (200 mL), washed with saturated sodium bicarbonate aqueous solution (50 mL) and brine (40 mL), dried over sodium sulfate, filtered, and concentrated to give N-(4-methyl-3-(3-(9-(tetrahydro-2H-pyran-2-yl)-9H-purin-6-yl)pyridin-2-ylamino)phenyl)-6-(trifluoromethyl)picolinamide (170 mg, 0.30 mmol, 97%) as a yellow solid. MS (ESI) m/z 574.8 [M+H]⁺

Step 4. To a mixture of N-(4-methyl-3-(3-(9-(tetrahydro-2H-pyran-2-yl)-9H-purin-6-yl)pyridin-2-yl amino)phenyl)-6-(trifluoromethyl)picolinamide (180 mg, 0.31 mmol) in tetrahydrofuran (5 mL) was added hydrochloric acid (6 N, 6 mL) at room temperature. Then the reaction mixture was stirred at room temperature for 16 h. The reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-(3-(9H-purin-6-yl)pyridin-2-ylamino)-4-methylphenyl)-6-(trifluoromethyl) picolinamide (25 mg, 0.05 mmol, 17%) as a yellow solid. ¹H NMR (400 MHZ, DMSO-d₆) δ 13.88 (s, 1H), 12.36 (s, 1H), 10.33 (s, 1H), 9.84 (m, 1H), 9.10 (s, 1H), 8.73-8.73 (m, 2H), 8.40-8.34 (m, 3H), 8.17 (m, 1H), 7.49 (m, 1H), 7.27 (m, 1H), 7.05 (m, 1H), 2.45 (s, 3H). MS (ESI) m/z 491.1 [M+H]⁺

### Example 5. Synthesis of N-(3-((3-(9H-purin-6-yl)pyridin-2-yl)amino)-4-methylphenyl)-4-chlorobenzamide (Compound I-4)

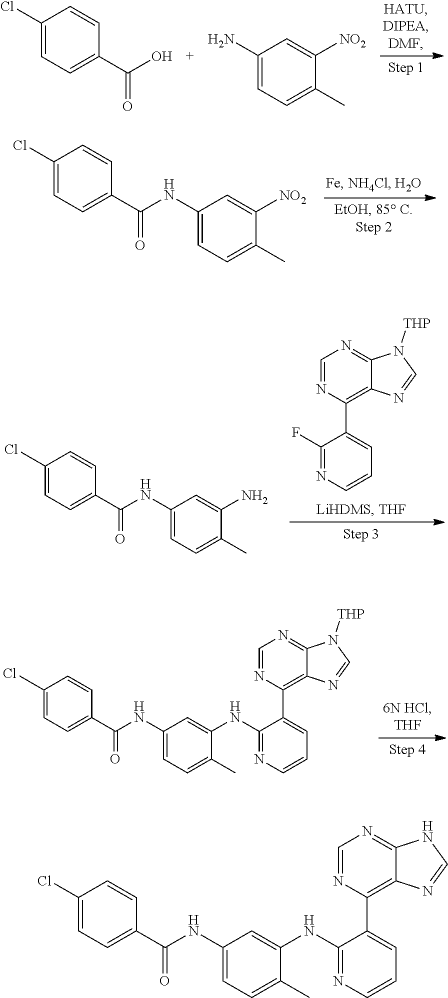

Step 1. A solution of 4-chlorobenzoic acid (2 g, 12.8 mmol), 4-methyl-3-nitroaniline (1.95 g, 12.8 mmol) and 1-[bis(dimethylamino)methylene]-1H-1,2,3-triazolo[4,5-b]pyridinium 3-oxid hexafluorophosphate (7.3 g, 19.2 mmol), and N,N-diisopropylethylamine (4.94 g, 32.8 mmol) in N, N-dimethylformamide (20 mL) was stirred at 20° C. for 8 h. The reaction mixture was extracted with ethyl acetate (50 mL×2), washed with brine (40 mL×3), dried, concentrated, and triturated with ethyl acetate to afford 4-chloro-N-(4-methyl-3-nitrophenyl)benzamide (1.5 g, 5.2 mmol, 41%) as a yellow solid. ¹H NMR (400 MHZ, DMSO-d6) δ 10.67 (s, 1H), 8.55 (d, J=2.2 Hz, 1H), 8.00 (dd, J=12.2, 5.4 Hz, 3H), 7.64 (d, J=8.5 Hz, 2H), 7.50 (d, J=8.5 Hz, 1H), 2.50 (s, 6H). MS (ESI) m/z 291.0 [M+H]⁺

Step 2. To a solution of 4-chloro-N-(4-methyl-3-nitrophenyl)benzamide (1.2 g, 4.14 mmol) in ethanol (120 mL) was added iron (1.16 g, 20.7 mmol) and a solution of ammonium chloride (2.21 g, 41.4 mmol) in water (13 mL). The mixture was stirred at 85° C. for 2 h. The heated reaction mixture was filtered, washing with ethanol and dichloromethane. The filtrate was concentrated, extracted with ethyl acetate (100 mL×2), washed with water and brine, dried, and concentrated to afford N-(3-amino-4-methylphenyl)-4-chlorobenzamide (1.2 g, 3.84 mmol, 93%) as a yellow solid. MS (ESI) m/z 261.1 [M+H]⁺

Step 3. To a solution of N-(3-amino-4-methylphenyl)-4-chlorobenzamide (100 mg, 0.38 mmol) and 6-(2-fluoropyridin-3-yl)-9-(tetrahydro-2H-pyran-2-yl)-9H-purine (114 mg, 0.38 mmol) in tetrahydrofuran (10 mL) was added 1.6 M lithium hexamethyldisilazide (1.2 mL, 1.9 mmol) at 0° C. The mixture was stirred at 0° C.-25° C. for 2 h. The reaction was quenched with saturated solution of ammonium chloride solution and extracted with ethyl acetate (20 mL×2). The combined organic layers were washed with brine, dried, and concentrated to afford 4-chloro-N-(4-methyl-3-((3-(9-(tetrahydro-2H-pyran-2-yl)-9H-purin-6-yl)pyridin-2-yl)amino)phenyl) benzamide (200 mg, 0.37 mmol, 98%) as a yellow solid. MS (ESI) m/z 540.2 [M+H]⁺

Step 4. To a solution of 4-chloro-N-(4-methyl-3-((3-(9-(tetrahydro-2H-pyran-2-yl)-9H-purin-6-yl)pyridin-2-yl) amino)phenyl)benzamide (200 mg, 0.37 mmol) in tetrahydrofuran (21 mL) was added 6 N hydrochloric acid (3.5 mL) at 25° C. The mixture was stirred at 25° C. for 1 h and then concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-4-chlorobenzamide (63.5 mg, 0.14 mmol, 39%) as an orange-red solid. ¹H NMR (400 MHZ, DMSO-d₆) δ 13.83 (s, 1H), 12.32 (s, 1H), 10.31 (s, 1H), 9.79 (d, J=5.4 Hz, 1H), 9.09 (s, 1H), 8.74 (s, 1H), 8.68 (s, 1H), 8.37 (t, J=4.0 Hz, 1H), 8.01 (d, J=8.2 Hz, 2H), 7.61 (d, J=8.2 Hz, 2H), 7.47 (d, J=8.1 Hz, 1H), 7.23 (d, J=8.2 Hz, 1H), 7.04 (dd, J=7.6, 4.7 Hz, 1H), 2.44 (s, 3H). MS (ESI) m/z 456.2 [M+H]⁺

### Example 6. Synthesis of N-(3-((3-(9H-Purin-6-yl)pyridin-2-yl)amino)-4-methylphenyl)-2-(trifluoromethyl)isonicotinamide (Compound I-5)

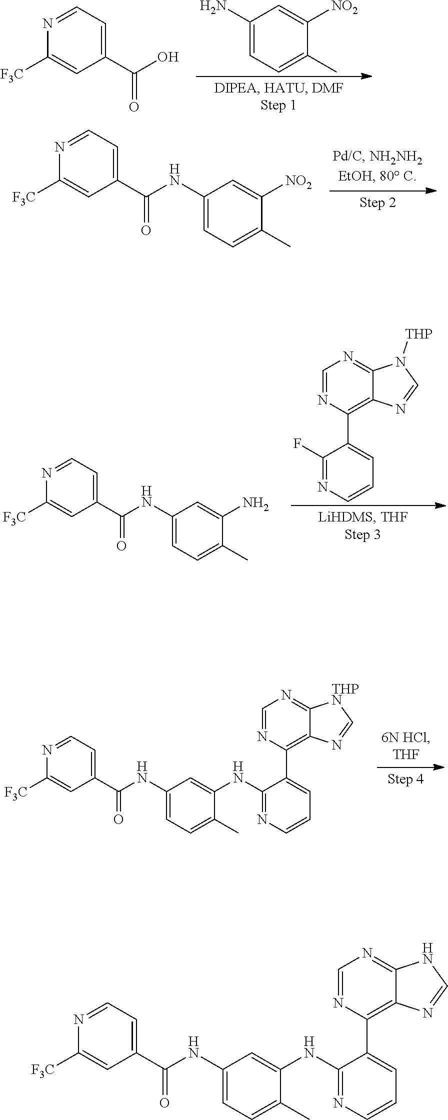

Step 1. To a mixture of 2-(trifluoromethyl)isonicotinic acid (2.0 g, 10.5 mmol) in N,N-dimethylformamide (15 mL) was added N,N-diisopropylethylamine (4.1 g, 31.4 mmol, 5.2 mL), 4-methyl-3-nitroaniline (1.6 g, 10.5 mmol) and 1-[bis(dimethylamino)methylene]-1H-1,2,3-triazolo[4,5-b]pyridinium 3-oxid hexafluorophosphate (6.0 g, 15.7 mmol) at room temperature. Then the reaction mixture was stirred at room temperature for 1 h. The reaction mixture was extracted with ethyl acetate (100 mL×3). The combined organic layer was washed with saturated sodium bicarbonate aqueous solution (50 mL) and brine (40 mL), dried over sodium sulfate, filtered, and concentrated. The residue was purified by flash chromatography (petroleum ether/ethyl acetate=3/1) to give N-(4-methyl-3-nitrophenyl)-2-(trifluoromethyl)isonicotinamide (2.6 g, 8.0 mmol, 76%) as a white solid. MS (ESI) m/z 326.0 [M+H]⁺

Step 2. A mixture of N-(4-methyl-3-nitrophenyl)-2-(trifluoromethyl)isonicotinamide (1.7 g, 5.23 mmol), palladium (10% on activated carbon, 1.7 g) and hydrazine (34.8 g, 696 mmol) in ethanol (20 mL) was stirred at 80° C. for 1 h. After cooling to room temperature, the mixture was diluted with ethyl acetate (40 mL) and filtered through a celite pad. The filtrate was concentrated and dried to give N-(3-amino-4-methylphenyl)-2-(trifluoromethyl)isonicotinamide (1.2 g, 4.07 mmol, 33%) as a white solid. MS (ESI) m/z 296.0 [M+H]⁺

Step 3. Lithium hexamethyldisilazide (1.6 M in tetrahydrofuran, 15.3 mL, 24.4 mmol) was added to a mixture of N-(3-amino-4-methylphenyl)-2-(trifluoromethyl)isonicotinamide (1.2 g, 4.07 mmol) and 6-(2-fluoropyridin-3-yl)-9-(tetrahydro-2H-pyran-2-yl)-9H-purine (1.22 g, 4.07 mmol) in tetrahydrofuran (10 mL) at 0° C. under nitrogen. Then the reaction mixture was stirred at room temperature for 2 h. The reaction mixture was diluted with water and extracted with ethyl acetate (200 mL). The organic layer was washed with saturated sodium bicarbonate aqueous solution (50 mL) and brine (40 mL), dried over sodium sulfate, filtered, and concentrated under reduced pressure. The residue was purified by flash chromatography (petroleum ether/ethyl acetate=10/1) to give N-(4-methyl-3-((3-(9-(tetrahydro-2H-pyran-2-yl)-9H-purin-6-yl)pyridin-2-yl)amino)phenyl)-2-(trifluoromethyl)isonicotinamide (770 mg, 1.34 mmol, 33%) as a white solid. MS (ESI) m/z 575.0 [M+H]⁺

Step 4. To a mixture of N-(4-methyl-3-((3-(9-(tetrahydro-2H-pyran-2-yl)-9H-purin-6-yl)pyridin-2-yl)amino)phenyl)-2-(trifluoromethyl)isonicotinamide (440 mg, 0.70 mmol) in tetrahydrofuran (10 mL) was added hydrochloric acid (6 N, 15 mL) at room temperature. Then the reaction mixture was stirred at room temperature for 16 h. The reaction mixture was adjusted to pH=10.0 with 1N NaOH. The mixture was extracted with ethyl acetate (50 mL). The organic layer was washed with saturated sodium bicarbonate aqueous solution (50 mL) and brine (25 mL), dried over sodium sulfate, filtered, and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-2-(trifluoromethyl)isonicotinamide (70.1 mg, 0.145 mmol, 21%) as a yellow solid. ¹H NMR (500 MHZ, DMSO-d₆) δ 13.85 (s, 1H), 12.36 (s, 1H), 10.65 (s, 1H), 9.83 (s, 1H), 9.09 (s, 1H), 8.98 (d, J=5.0 Hz, 1H), 8.72 (s, 2H), 8.36-8.38 (m, 2H), 8.21 (d, J=5.0 Hz, 1H), 7.49 (q, J=2.0 Hz, 1H), 7.27 (d, J=8.5 Hz, 1H), 7.03-7.06 (m, 1H), 2.46 (s, 3H). MS (ESI) m/z 491.0 [M+H]⁺

### Example 7. Synthesis of N-(3-(3-(9H-purin-6-yl)pyridin-2-ylamino)-4-methylphenyl)-benzamide (Compound I-6)

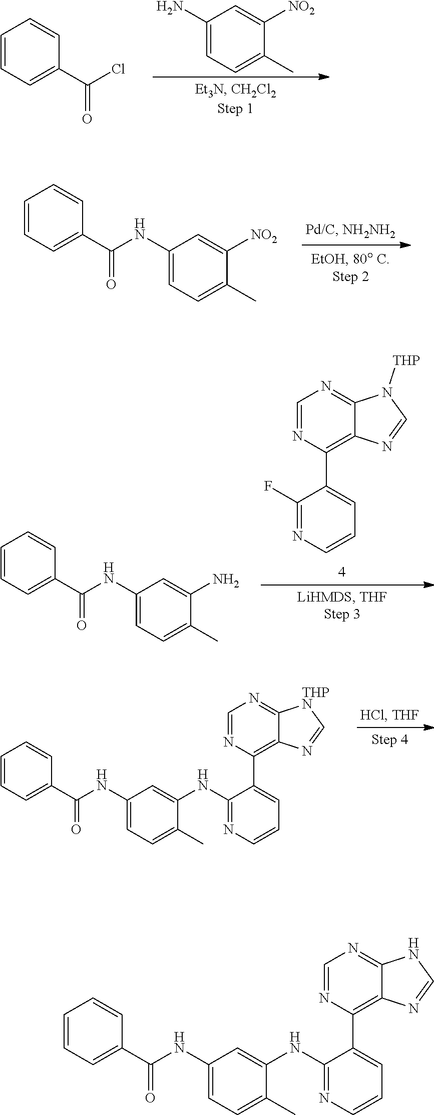

Step 1. To a mixture of benzoyl chloride (1.0 g, 7.14 mmol) in dichloromethane (15 mL) was added 4-methyl-3-nitroaniline (1.3 g, 8.57 mmol) and triethylamine (2.1 g 21.42 mmol) at room temperature. Then the reaction mixture was stirred at room temperature for 2 h. The reaction was diluted with ethyl acetate (20 mL), washed with saturated sodium bicarbonate aqueous solution (50 mL) and brine (40 mL), dried over sodium sulfate, filtered, and concentrated. The residue was purified by flash chromatography (petroleum ether/ethyl acetate=4/1) to give N-(4-methyl-3-nitrophenyl) benzamide (1.6 g, 6.25 mmol, 88%) as a white solid. MS (ESI) m/z 257.0 [M+H]⁺

Step 2. To a mixture of N-(4-methyl-3-nitrophenyl)benzamide (1.4 g, 5.47 mmol) in ethanol (15 mL) was added palladium (10% Pd on activated carbon, 100 mg). The mixture was stirred at room temperature for 0.5 h under hydrogen atmosphere. The mixture was diluted with ethyl acetate (40 mL) and filtered through celite. The filtrate was concentrated and dried to give N-(3-amino-4-methylphenyl)benzamide (1.2 g, 5.31 mmol, 97%) as a white solid. MS (ESI) m/z 227.1 [M+H]⁺

Step 3. Lithium hexamethyldisilazide (1.6 M in tetrahydrofuran, 8.3 mL, 13.25 mmol) was added to a mixture of N-(3-amino-4-methylphenyl)benzamide (500 mg, 2.21 mmol) and 6-(2-fluoropyridin-3-yl)-9-(tetrahydro-2H-pyran-2-yl)-9H-purine (661 mg, 2.21 mmol) in tetrahydrofuran (20 mL) at 0° C. under nitrogen. Then the reaction mixture was stirred at room temperature for 2 h. The reaction was diluted with ethyl acetate (200 mL), washed with saturated sodium bicarbonate aqueous solution (50 mL) and brine (40 mL), dried over sodium sulfate, filtered, and concentrated to give N-(4-methyl-3-((3-(9-(tetrahydro-2H-pyran-2-yl)-9H-purin-6-yl)pyridin-2-yl)amino) phenyl)benzamide (1.0 g, 1.98 mmol, 90%) as a yellow solid. MS (ESI) m/z 506 [M+H]⁺

Step 4. A mixture of N-(4-methyl-3-((3-(9-(tetrahydro-2H-pyran-2-yl)-9H-purin-6-yl)pyridin-2-yl) amino)phenyl) benzamide (300 mg, 0.6 mmol) in tetrahydrofuran (5 mL) was added hydrochloric acid (6 N, 6 mL) at room temperature. Then the reaction mixture was stirred at room temperature for 16 h. The reaction was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)benzamide (15 mg, 0.0356 mmol, 6%) as a yellow solid. ¹H NMR (400 MHz, DMSO-d₆) δ 13.87 (s, 1H), 12.31 (s, 1H), 10.22 (s, 1H), 9.82 (s, 1H), 9.09 (s, 1H), 8.77-8.59 (m, 2H), 8.35 (dd, J=4.7, 1.9 Hz, 1H), 8.11-7.84 (m, 2H), 7.46-7.59 (m, 4H), 7.23 (d, J=8.3 Hz, 1H), 7.03 (dd, J=7.9, 4.7 Hz, 1H), 2.43 (s, 3H). MS (ESI) m/z 422 [M+H]⁺

### Example 8. Synthesis of N-(3-(3-(9H-purin-6-yl)pyridin-2-ylamino)-4-fluorophenyl)-3-(2-cyanopropan-2-yl)benzamide (Compound II-2)

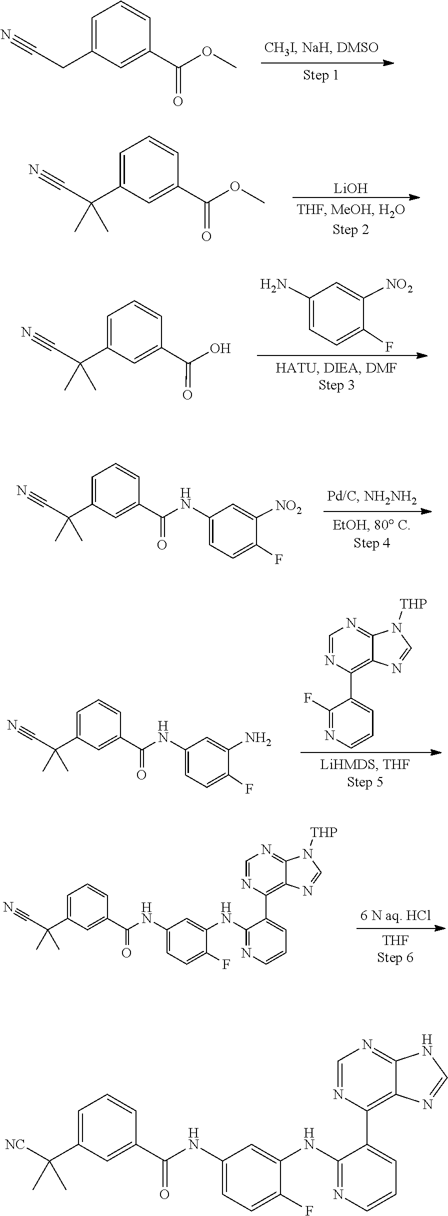

Step 1. A solution of 3-cyanomethyl-benzoic acid methyl ester (5.0 g, 28.5 mmol) in dimethyl sulfoxide (80 mL) was treated with sodium hydride (60% dispersion in oil, 3.4 g, 85.5 mmol). To this deep-red suspension was then added methyl iodide (5.5 mL, 85.5 mmol) dropwise at 20° C. This resulting mixture was stirred at 20° C. for 16 h. The reaction mixture was quenched with water (200 mL) and extracted with ethyl acetate (200 mL). The combined organic layers were dried with sodium sulfate, filtered, and concentrated in vacuo. The crude residue was purified by flash chromatography (petroleum ether/ethyl acetate=10/1) to give methyl 3-(2-cyanopropan-2-yl)benzoate (4.3 g, 21.16 mmol, 74%) as a colorless oil. ¹H NMR (400 MHZ, DMSO-d₆) δ 8.08 (t, J=1.9 Hz, 1H), 7.97-7.93 (m, 1H), 7.86-7.81 (m, 1H), 7.61 (t, J=7.8 Hz, 1H), 3.88 (s, 3H), 1.72 (s, 6H). MS (ESI) m/z 221.3 [M+H]⁺

Step 2. To a solution of methyl 3-(2-cyanopropan-2-yl)benzoate (4.0 g, 19.7 mmol) in tetrahydrofuran (42 mL) were added lithium hydroxide monohydrate (1.4 g, 33.5 mmol), methanol (14 mL), and water (14 mL). The mixture was stirred at 20° C. for 5 h. The solvent was evaporated under reduced pressure, and the residue was diluted with water (21 mL). The mixture was adjusted to pH 3 by adding 1 N hydrochloric acid slowly. The resulting white precipitate was collected by filtration and washed with water (100 mL) to afford 3-(2-cyanopropan-2-yl)benzoic acid (3.48 g, 18.3 mmol, 93%) as a white solid. ¹H NMR (400 MHZ, DMSO-d₆) δ 13.20 (s, 1H), 8.13-8.01 (m, 1H), 7.96-7.89 (m, 1H), 7.82-7.74 (m, 1H), 7.66-7.52 (m, 1H), 1.72 (s, 6H). MS (ESI) m/z 190.1 [M+H]⁺

Step 3. A solution of 3-(2-cyanopropan-2-yl)benzoic acid (811 mg, 4.29 mmol), 4-fluoro-3-nitroaniline (693 mg, 4.44 mmol), 1-[bis(dimethylamino)methylene]-1H-1,2,3-triazolo[4,5-b]pyridinium 3-oxid hexafluorophosphate (2.4 g, 6.35 mmol) and N,N-diisopropylethylamine (1.6 g, 12.7 mmol) in N,N-dimethylformamide (10 mL) was stirred at 20° C. for 16 h. The mixture was diluted with ether acetate (100 mL) and washed with water (100 mL). The organic phase was washed with brine (40 mL), dried over sodium sulfate, filtered, and concentrated. The residue was purified by flash chromatography (ethyl acetate/petroleum ether=30/100) to afford 3-(2-cyanopropan-2-yl)-N-(4-fluoro-3-nitrophenyl) benzamide (1.06 g, 3.24 mmol, 76%) as a yellow solid. MS (ESI) m/z 328.0 [M+H]⁺

Step 4. A mixture of 3-(2-cyanopropan-2-yl)-N-(4-fluoro-3-nitrophenyl)benzamide (1.2 g, 3.7 mmol), hydrazine hydrate (6 mL) and palladium (10% on activated carbon, 600 mg) in ethanol (60 mL) was stirred at 80° C. for 1 h. The reaction mixture was cooled, filtered, and concentrated. The residue was diluted with ethyl acetate (30 mL) and washed with water (30 mL). The organic phase was washed with brine (40 mL), dried over sodium sulfate, filtered, and concentrated to give N-(3-amino-4-fluorophenyl)-3-(2-cyanopropan-2-yl) benzamide (1.07 g, 3.6 mmol, 97%) as a yellow solid. MS (ESI) m/z 298.0 [M+H]⁺

Step 5. To a solution of N-(3-amino-4-fluorophenyl)-3-(2-cyanopropan-2-yl)benzamide (150 mg, 0.5 mmol), 6-(2-fluoropyridin-3-yl)-9-(tetrahydro-2H-pyran-2-yl)-9H-purine (150 mg, 0.5 mmol) in tetrahydrofuran (10 mL) was added lithium hexamethyldisilazide (1.6 mL, 2.5 mmol, 1.6 M in tetrahydrofuran) at 0° C. slowly. The mixture was stirred at 20° C. for 5 h. The reaction was quenched with saturated ammonium chloride solution (20 mL) at 0° C. and extracted with ethyl acetate (50 mL×3). The combined organic layer was washed with brine (50 mL), dried over sodium sulfate filtered, and concentrated under reduced pressure. The crude residue was purified flash chromatography (petroleum ether/ethyl acetate=1/100) to give 3-(2-cyanopropan-2-yl)-N-(4-fluoro-3-(3-(9-(tetrahydro-2H-pyran-2-yl)-9H-purin-6-yl)pyridin-2ylamino)phenyl)benzamide (80 mg, 0.14 mmol, 28%) as a yellow solid. MS (ESI) m/z 576.9 [M+H]⁺

Step 6. To a solution of 3-(2-cyanopropan-2-yl)-N-(4-fluoro-3-(3-(9-(tetrahydro-2H-pyran-2-yl)-9H-purin-6-yl)pyridin-2-ylamino)phenyl)benzamide (60 mg, 0.1 mmol) in tetrahydrofuran (4 mL) was added hydrochloric acid (6 N, 1 mL) at 20° C., and the mixture was stirred at 20° C. for 2 h. After completed, the reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-(3-(9H-purin-6-yl)pyridin-2-ylamino)-4-fluorophenyl)-3-(2-cyanopropan-2-yl)benzamide (26.3 mg, 0.09 mmol, 38%) as a yellow solid. ¹H NMR (400 MHZ, DMSO-d₆) δ 13.88 (s, 1H), 12.85 (s, 1H), 10.41 (s, 1H), 9.83 (s, 1H), 9.06 (s, 1H), 9.02-8.90 (m, 1H), 8.75 (s, 1H), 8.43 (dd, J=4.7, 1.9 Hz, 1H), 8.07 (t, J=1.9 Hz, 1H), 7.97 (d, J=7.7 Hz, 1H), 7.84-7.72 (m, 1H), 7.61 (t, J=7.8 Hz, 1H), 7.53-7.43 (m, 1H), 7.31 (dd, J=11.1, 8.8 Hz, 1H), 7.12 (dd, J=7.9, 4.7 Hz, 1H), 1.77 (s, 6H). MS (ESI) m/z 492.9 [M+H]⁺

### Example 9. Synthesis of N-(3-(3-(9H-purin-6-yl)pyridin-2-ylamino)-4-methylphenyl)-2-(6-(trifluoromethyl)pyridin-2-yl)acetamide (Compound I-7)

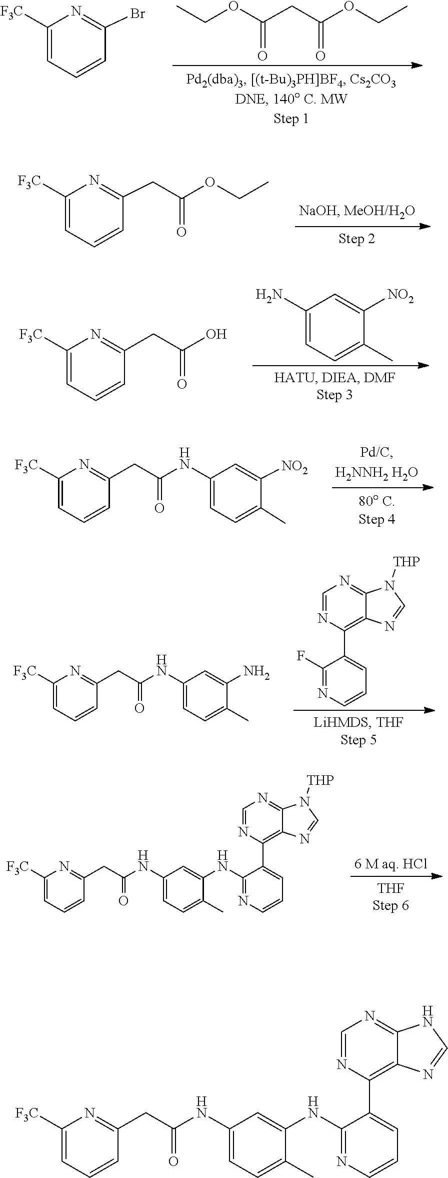

Step 1. Cesium carbonate (433 mg, 1.329 mmol) was added to a solution of 2-bromo-6-(trifluoromethyl)pyridine (200 mg, 0.885 mmol), diethyl malonate (170 mg, 1.062 mmol), tris(dibenzylideneacetone)dipalladium(0) (17 mg, 0.019 mmol) and tri-tert-butylphosphonium tetrafluoroborate (26 mg, 0.09 mmol) in 1,2-dimethoxyethane (3 mL) at room temperature under nitrogen. The reaction mixture was heated to 140° C. for 0.5 h in a microwave reactor. After cooling to room temperature, the reaction mixture was treated with saturated solution of ammonium chloride (20 mL) and extracted with ethyl acetate (30 mL×2). The organic layers were dried over sodium sulfate, filtered, and concentrated. The residue was purified by silica gel column, eluting with petroleum ether/ethyl acetate=9/1 to give ethyl 2-(6-(trifluoromethyl)pyridin-2-yl)acetate (100 mg, 0.429 mmol, 48%) as a brown oil. MS (ESI) m/z 233.9 [M+H]⁺

Step 2. Sodium hydroxide (52 mg, 1.3 mmol) in water (1.5 mL) was added to a solution of ethyl 2-(6-(trifluoromethyl)pyridin-2-yl)acetate (100 mg, 0.429 mmol) in methanol (3 mL) at room temperature. The reaction mixture was stirred at room temperature for 4 h. The reaction mixture was acidified with concentrated hydrochloric acid until pH=3. The solution was concentrated and dried to give 2-(6-(trifluoromethyl)pyridin-2-yl)acetic acid (87 mg, 0.424 mmol, 99%) as a white solid. The material was used without further purification. MS (ESI) m/z 206.0 [M+H]⁺

Step 3. 1-[bis(dimethylamino)methylene]-1H-1,2,3-triazolo[4,5-b]pyridinium 3-oxid hexafluorophosphate (2.419 g, 6.36 mmol) was added to a solution of 2-(6-(trifluoromethyl)pyridin-2-yl)acetic acid (870 mg, 4.24 mmol), 4-methyl-3-nitroaniline (774 mg, 5.09 mmol) and N,N-diisopropylethylamine (2.192 g, 16.96 mmol) in N,N-dimethylformamide (15 mL) at room temperature under nitrogen. The reaction mixture was stirred at room temperature for 1 h. The reaction mixture was treated with water (80 mL) and extracted with ethyl acetate (70 mL×3). The organic layers were washed with water (50 mL×2) and brine (40 mL), dried over sodium sulfate, filtered, and concentrated. The residue was purified by silica gel column, eluting with petroleum ether/ethyl acetate=3/1 to give N-(4-methyl-3-nitrophenyl)-2-(6-(trifluoromethyl)pyridin-2-yl)acetamide (500 mg, 1.474 mmol, 35%) as a yellow solid. MS (ESI) m/z 340.0 [M+H]⁺

Step 4. Palladium (10% Pd on activated carbon, 100 mg) was added to a solution of N-(4-methyl-3-nitrophenyl)-2-(6-(trifluoromethyl)pyridin-2-yl)acetamide (500 mg, 1.474 mmol) in hydrazine hydrate solution (2 mL) and ethanol (30 mL) at room temperature. The mixture was stirred at 80° C. for 1 h. After cooling to room temperature, the reaction mixture was filtered through celite. The filtrate was diluted with ethyl acetate (300 mL), washed with water (50 mL) and brine (50 mL), dried over sodium sulfate, filtered, and concentrated to give N-(3-amino-4-methylphenyl)-2-(6-(trifluoromethyl)pyridin-2-yl)acetamide (300 mg, 0.97 mmol, 66%) as a white solid. MS (ESI) m/z 310.1 [M+H]⁺

Step 5. Lithium hexamethyldisilazide (1.6 M in tetrahydrofuran, 2.2 mL, 3.52 mmol) was added to a solution of N-(3-amino-4-methylphenyl)-2-(6-(trifluoromethyl)pyridin-2-yl)acetamide (300 mg, 0.97 mmol) and 6-(2-fluoropyridin-3-yl)-9-(tetrahydro-2H-pyran-2-yl)-9H-purine (290 mg, 0.97 mmol) in tetahydrofuran (15 mL) at 0° C. The mixture was stirred at room temperature for 1 h. The reaction was quenched with water (60 mL) and extracted with ethyl acetate (80 mL×2). The organic layers were washed with brine (40 mL), dried over sodium sulfate, filtered, and concentrated. The crude was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(4-methyl-3-(3-(9-(tetrahydro-2H-pyran-2-yl)-9H-purin-6-yl)pyridin-2-ylamino)phenyl)-2-(6-(trifluoromethyl)pyridin-2-yl)acetamide (40 mg, 0.068 mmol, 7%) as a yellow solid. MS (ESI) m/z 589.1 [M+H]⁺

Step 6. 6 N Hydrochloric acid (2 mL) was added to a solution of N-(4-methyl-3-(3-(9-(tetrahydro-2H-pyran-2-yl)-9H-purin-6-yl)pyridin-2-ylamino)phenyl)-2-(6-(trifluoromethyl)-pyridin-2-yl)acetamide (35 mg, 0.059 mmol) in tetrahydrofuran (2 mL) at room temperature. The solution mixture was stirred at room temperature for 2 h. The reaction mixture was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to N-(3-(3-(9H-purin-6-yl)pyridin-2-ylamino)-4-methylphenyl)-2-(6-(trifluoromethyl)-pyridin-2-yl)acetamide (20.4 mg, 0.04 mmol, 68%) as a yellow solid. ¹H NMR (500 MHz, DMSO-d₆) δ 13.73 (br s, 1H), 12.32 (s, 1H), 10.26 (s, 1H), 9.79 (m, 1H), 9.06 (s, 1H), 8.71 (s, 1H), 8.53 (d, J=2.0 Hz, 1H), 8.35 (m, 1H), 8.08 (m, 1H), 7.81 (d, J=7.6 Hz, 1H), 7.75 (d, J=7.6 Hz, 1H), 7.39 (m, 1H), 7.17 (d, J=8.0 Hz, 1H), 7.04 (m, 1H), 4.00 (s, 2H), 2.41 (s, 3H). MS (ESI) m/z 505.1 [M+H]⁺

### Example 10. Synthesis of N-(5-(3-(9H-purin-6-yl)pyridin-2-ylamino)-6-methylpyridin-3-yl)-3-(2-cyanopropan-2-yl)benzamide (Compound I-8)

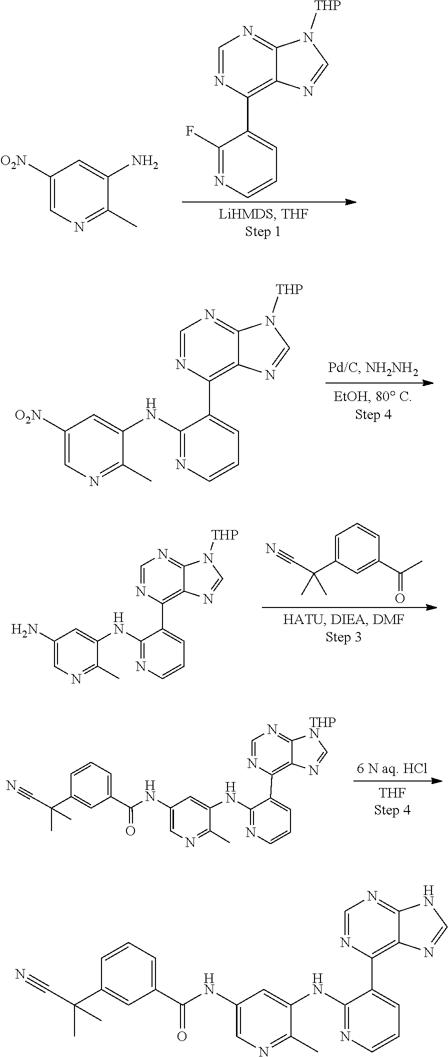

Step 1. To a solution of 2-methyl-5-nitropyridin-3-amine (350 mg, 2.23 mmol), 6-(2-fluoropyridin-3-yl)-9-(tetrahydro-2H-pyran-2-yl)-9H-purine (667 mg, 2.23 mmol) in tetrahydrofuran (20 mL) was added lithium hexamethyldisilazide (1.6 M in tetrahydrofuran, 7 mL, 11 mmol) at 0° C. slowly. The mixture was stirred at 0° C. for 5 h. The reaction was quenched with saturated ammonium chloride solution (20 mL) at 0° C. and extracted with ethyl acetate (50 mL×3). The combined organic layer was washed with brine (50 mL), dried over sodium sulfate and filtered. The filtrate was concentrated under reduced pressure. The crude residue was purified by flash chromatography eluting with ethyl acetate to give N-(2-methyl-5-nitropyridin-3-yl)-3-(9-(tetrahydro-2H-pyran-2-yl)-9H-purin-6-yl)pyridin-2-amine (130 mg, 0.3 mmol, 13%) as a yellow solid. MS (ESI) m/z 433.0 [M+H]⁺

Step 2. A mixture of N-(2-methyl-5-nitropyridin-3-yl)-3-(9-(tetrahydro-2H-pyran-2-yl)-9H-purin-6-yl)pyridin-2-amine (110 mg, 0.25 mmol), hydrazine hydrate (0.6 mL) and palladium (10% on activated carbon, 55 mg) in ethanol (12 mL) was stirred at 80° C. for 1 h. The reaction mixture was cooled, filtered, and concentrated. The residue was diluted with ethyl acetate (50 mL) and washed with water (50 mL) and brine (50 mL), dried over sodium sulfate, filtered, and concentrated to give 2-methyl-N3-(3-(9-(tetrahydro-2H-pyran-2-yl)-9H-purin-6-yl)pyridin-2-yl)pyridine-3,5-diamine (102 mg, 0.25 mmol, 99%) as a yellow solid. MS (ESI) m/z 403.0 [M+H]⁺

Step 3. A solution of 2-methyl-N3-(3-(9-(tetrahydro-2H-pyran-2-yl)-9H-purin-6-yl)pyridin-2-yl) pyridine-3,5-diamine (120 mg, 0.3 mmol), 3-(2-cyanopropan-2-yl)benzoic acid (57 mg, 0.3 mmol), 1-[bis(dimethylamino)methylene]-1H-1,2,3-triazolo[4,5-b]pyridinium 3-oxid hexafluorophosphate (171 mg, 0.45 mmol) and N,N-diisopropylethylamine (116 mg, 0.9 mmol) in N,N-dimethylformamide (3 mL) was stirred at 20° C. for 1 h. The mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 3-(2-cyanopropan-2-yl)-N-(6-methyl-5-(3-(9-(tetrahydro-2H-pyran-2-yl)-9H-purin-6-yl)pyridin-2-ylamino)pyridin-3-yl)benzamide (90 mg, 0.18 mmol, 60%) as a yellow solid. MS (ESI) m/z 573.9 [M+H]⁺

Step 4. To a solution of 3-(2-cyanopropan-2-yl)-N-(6-methyl-5-(3-(9-(tetrahydro-2H-pyran-2-yl)-9H-purin-6-yl)pyridin-2-ylamino)pyridin-3-yl)benzamide (90 mg, 0.16 mmol) in tetrahydrofuran (8 mL) was added hydrochloric acid (6 N, 2 mL) at 20° C., and stirred at 20° C. for 1 h. After completed, the reaction mixture was concentrated and diluted with tetrahydrofuran (5 mL). The pH was adjusted to 8 with saturated sodium bicarbonate solution. The resulting crystalline was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(5-(3-(9H-purin-6-yl)pyridin-2-ylamino)-6-methylpyridin-3-yl)-3-(2-cyanopropan-2-yl)benzamide (30.9 mg, 0.063 mmol, 39%) as a yellow solid. ¹H NMR (400 MHZ, DMSO-d₆) δ 13.90 (s, 1H), 12.58 (s, 1H), 10.47 (s, 1H), 9.86 (s, 1H), 9.18 (d, J=2.4 Hz, 1H), 9.12 (s, 1H), 8.75 (s, 1H), 8.57 (d, J=2.3 Hz, 1H), 8.40 (dd, J=4.7, 1.9 Hz, 1H), 8.09 (t, J=1.9 Hz, 1H), 8.02-7.97 (m, 1H), 7.82-7.74 (m, 1H), 7.62 (t, J=7.8 Hz, 1H), 7.11 (dd, J=7.9, 4.7 Hz, 1H), 2.69 (s, 3H), 1.77 (s, 6H). MS (ESI) m/z 489.9 [M+H]⁺

### Example 11. Synthesis of N-(3-(3-(9H-purin-6-yl)pyridin-2-ylamino)-4-methylphenyl)-3-(1-cyanocyclopropyl)benzamide (Compound I-9)

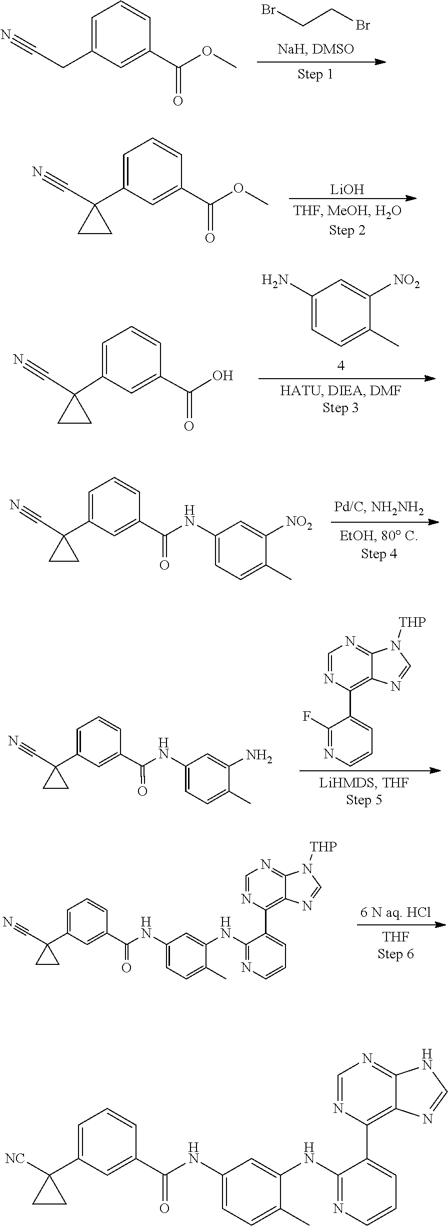

Step 1. A solution of methyl 3-(cyanomethyl)benzoate (2.7 g, 1.71 mmol) in dimethyl sulfoxide (40 mL) was treated with sodium hydride (60% dispersion in oil, 1.8 g, 46.2 mmol). To this deep-red suspension was then added 1,2-dibromoethane (4.3 g, 23.1 mmol) dropwise at 20° C. This resulting mixture was stirred at 20° C. for 16 h. The reaction mixture was quenched with water (200 mL) and extracted with ethyl acetate (100 mL). The combined organic layers were dried with sodium sulfate, filtrated and concentrated in vacuo, The pale yellow residue was dissolved in dichloromethane and purified by flash chromatography (petroleum ether/ethyl acetate=10/1) to give methyl 3-(1-cyanocyclopropyl)benzoate (620 mg, 3.08 mmol, 20%) as a white solid. MS (ESI) m/z 202.1 [M+H]⁺

Step 2. A solution of methyl 3-(1-cyanocyclopropyl)benzoate (650 mg, 3.2 mmol), lithium hydroxide (230 mg, 5.5 mmol) in tetrahydrofuran (7 mL), water (3 mL) and methanol (3 mL) was stirred at 20° C. for 5 h under argon. The reaction mixture was adjusted to pH=3 with 2 N hydrochloric acid. The mixture was concentrated, and the residue was diluted with ethyl acetate (25 mL), then washed with water (25 mL) and brine (25 mL), dried over sodium sulfate, filtered, and concentrated. The residue was purified by flash chromatography (dichloromethane/methanol=20/1) to get 3-(2-cyanopropan-2-yl)benzoic acid (500 mg, 2.67 mmol, 84%) as a yellow solid. MS (ESI) m/z 188.0 [M+H]⁺

Step 3. A solution of 3-(1-cyanocyclopropyl)benzoic acid (500 mg, 2.67 mmol), 4-methyl-3-nitroaniline (431 mg, 2.84 mmol), 1-[bis(dimethylamino)methylene]-1H-1,2,3-triazolo[4,5-b]pyridinium 3-oxid hexafluorophosphate (1.62 g, 4.26 mmol) and N,N-diisopropylethylamine (732 mg, 5.68 mmol) in N,N-dimethylformamide (10 mL) was stirred at 20° C. for 1 h. The mixture was diluted with ethyl acetate (50 mL), washed with water (50 mL) and brine (50 mL), dried over sodium sulfate, filtered, and concentrated. The residue was purified by flash chromatography (ethyl acetate/petroleum ether=1/5) to get N-(4-methyl-3-nitrophenyl)-4-(trifluoromethyl)-picolinamide (400 mg, 1.25 mmol, 47%) as a yellow solid. MS (ESI) m/z 322.2 [M+H]⁺

Step 4. A mixture of N-(4-methyl-3-nitrophenyl)-4-(trifluoromethyl)picolinamide (400 mg, 1.25 mmol), hydrazine hydrate (2.5 mL) and palladium (10% on activated carbon, 250 mg) in ethanol (50 mL) was stirred at 80° C. for 1 h. The reaction mixture was cooled, filtered, and concentrated. The residue was diluted with ethyl acetate (100 mL), washed with water (100 mL) and brine (60 mL), dried over sodium sulfate, filtered, and concentrated to give crude N-(3-amino-4-methylphenyl)-3-(1-cyanocyclopropyl)benzamide (350 mg, 1.20 mmol, 96%) as a yellow solid. MS (ESI) m/z 292.1 [M+H]⁺

Step 5. To a solution of N-(3-amino-4-methylphenyl)-3-(1-cyanocyclopropyl)benzamide (200 mg, 0.68 mmol), 6-(2-fluoropyridin-3-yl)-9-(tetrahydro-2H-pyran-2-yl)-9H-purine (204 mg, 0.68 mmol) in tetrahydrofuran (10 mL) was added lithium hexamethyldisilazide (1.6 M in tetrahydrofuran, 2.1 mL, 3.4 mmol) at 0° C. slowly. The mixture was stirred at 20° C. for 5 h. The reaction was quenched with saturated ammonium chloride solution (20 mL) at 0° C. and extracted with ethyl acetate (50 mL×3). The combined organic layers were washed with brine (50 mL), dried over sodium sulfate, and filtered. The filtrate was concentrated under reduced pressure. The residue was purified by flash chromatography (petroleum ether/ethyl acetate=1/10) to give 3-(1-cyanocyclopropyl)-N-(4-methyl-3-(3-(9-(tetrahydro-2H-pyran-2-yl)-9H-purin-6-yl) pyridin-2-ylamino)phenyl)benzamide (150 mg, 0.26 mmol, 38%) as a yellow solid. MS (ESI) m/z 571.3 [M+H]⁺ Step 6. To a solution of 3-(1-cyanocyclopropyl)-N-(4-methyl-3-(3-(9-(tetrahydro-2H-pyran-2-yl)-9H-purin-6-yl) pyridin-2-ylamino)phenyl)benzamide (100 mg, 0.18 mmol) in tetrahydrofuran (21 mL) was added hydrochloric acid (6 N, 3.5 mL) at 20° C., and the mixture was stirred at 20° C. for 5 h. After completed, the reaction mixture was concentrated and the residue was diluted with water (5 mL), the pH was adjusted to 8 with saturated sodium bicarbonate solution. The resulting crystalline was collected by filtration, washed with water (5 mL), and dried to afford N-(3-(3-(9H-purin-6-yl)pyridin-2-ylamino)-4-methylphenyl)-3-(1-cyanocyclopropyl) benzamide (70.7 mg, 0.15 mmol, 83%) as a white solid. ¹H NMR (400 MHZ, DMSO-d₆) δ 12.39 (s, 1H), 10.26 (s, 1H), 9.82 (d, J=6.4 Hz, 1H), 9.09 (s, 1H), 8.66-8.65 (m, 2H), 8.34-8.33 (m, 1H), 7.91-7.90 (m, 2H), 7.58-7.55 (m, 2H), 7.45-7.43 (m, 1H), 7.24-7.22 (m, 1H), 7.03-7.01 (m, 1H), 2.08 (s, 3H), 1.83-1.80 (m, 1H), 1.64-1.62 (m, 1H). MS (ESI) m/z 487.1 [M+H]⁺

### Example 12. Synthesis of N-(3-(3-(9H-purin-6-yl)pyridin-2-ylamino)-2,4-dimethylphenyl)-3-(2-cyanopropan-2-yl)benzamide (Compound I-10)

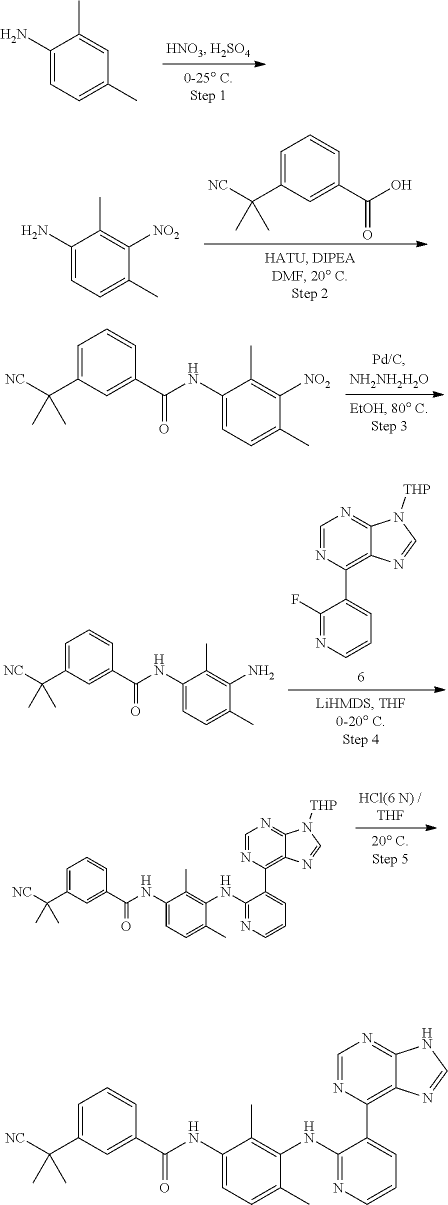

Step 1. To a solution of 2,4-dimethylaniline (6.2 mL, 49.5 mmol) in concentrated sulfuric acid (40 mL) at 0° C. was added fuming nitric acid (2.5 mL) and the mixture was stirred for 20 minutes at 4° C., and then for 30 minutes at room temperature. The reaction mixture was poured into ice water (600 mL), and the pH was adjusted to 10 with aqueous sodium hydroxide (5 N). The precipitate was suctioned off, washed with water, and dried to give the crude. The crude was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 2,4-dimethyl-3-nitroaniline (0.35 g, 2.11 mmol, 4%) as a yellow solid. MS (ESI) m/z 167.1 [M+H]⁺

Step 2. A solution of 2,4-dimethyl-3-nitroaniline (0.32 g, 1.7 mmol), 3-(2-cyanopropan-2-yl)benzoic acid (0.25 g, 1.5 mmol), 1-[bis(dimethylamino)methylene]-1H-1,2,3-triazolo[4,5-b]pyridinium 3-oxid hexafluorophosphate (0.97 g, 2.6 mmol) and N,N-diisopropylethylamine (0.44 g, 3.4 mmol) in dimethylformamide (5 mL) was stirred at 20° C. for 16 h. The mixture was diluted with ethyl acetate (150 mL) and washed with water (150 mL). The organic phase was washed with brine (40 mL), dried over sodium sulfate, filtered, and concentrated. The residue was purified by flash chromatography (ethyl acetate/petroleum ether=50/100) to afford 3-(2-cyanopropan-2-yl)-N-(2,4-dimethyl-3-nitrophenyl)benzamide (220 mg, 0.65 mmol, 39%) as a yellow solid. MS (ESI) m/z 338.0 [M+H]⁺

Step 3. A mixture of 3-(2-cyanopropan-2-yl)-N-(2,4-dimethyl-3-nitrophenyl)benzamide (0.22 g, 0.65 mmol), hydrazine hydrate (0.33 g) and palladium (10% on activated carbon, 50 mg) in ethanol (10 mL) was stirred at 80° C. for 1 h. The reaction mixture was cooled, filtered, and concentrated. The residue was diluted with ethyl acetate (100 mL) and washed with water (100 mL). The organic phase was washed with brine (60 mL), dried over sodium sulfate, filtered, and concentrated to give crude N-(3-amino-2,4-dimethylphenyl)-3-(2-cyanopropan-2-yl)benzamide (170 mg, 0.55 mmol, 85%) as a yellow solid. MS (ESI) m/z 308.0 [M+H]⁺

Step 4. To a solution of N-(3-amino-2,4-dimethylphenyl)-3-(2-cyanopropan-2-yl)benzamide (170 mg, 0.55 mmol), 6-(2-fluoropyridin-3-yl)-9-(tetrahydro-2H-pyran-2-yl)-9H-purine (166 mg, 0.55 mmol) in tetrahydrofuran (8 mL) was added lithium hexamethyldisilazide (1.7 mL, 2.75 mmol, 1.6 M in tetrahydrofuran) at 0° C. slowly. The mixture was stirred at 20° C. for 1 h. The reaction was quenched with ammonium chloride solution (20 mL) at 0° C. and extracted with ethyl acetate (50 mL×3), The combined organic layer was washed with brine (50 mL), dried over sodium sulfate, and filtered. The filtrate was concentrated under reduced pressure. The crude residue was purified by flash chromatography (100% ethyl acetate) to afford 3-(2-cyanopropan-2-yl)-N-(2,4-dimethyl-3-((3-(9-(tetrahydro-2H-pyran-2-yl)-9H-purin-6-yl)pyridin-2-yl)amino)-phenyl)benzamide (220 mg, 0.37 mmol, 68%) as a yellow solid. MS (ESI) m/z 587.0 [M+H]⁺

Step 5. To a solution of 3-(2-cyanopropan-2-yl)-N-(2,4-dimethyl-3-((3-(9-(tetrahydro-2H-pyran-2-yl)-9H-purin-6-yl)pyridin-2-yl)amino)phenyl)benzamide (150 mg, 0.26 mmol) in tetrahydrofuran (11 mL) was added hydrochloric acid (6 N, 3.5 mL) at 20° C., and stirred at 20° C. for 1 h. After completed, the reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-2,4-dimethylphenyl)-3-(2-cyanopropan-2-yl)benzamide (70.5 mg, 0.14 mmol, 55%) as a yellow solid. ¹H NMR (400 MHZ, DMSO-d₆) δ 13.83 (s, 1H), 11.62 (s, 1H), 10.08 (s, 1H), 9.74 (d, J=6.1 Hz, 1H), 9.05 (s, 1H), 8.71 (s, 1H), 8.22-8.07 (m, 2H), 7.99 (d, J=7.6 Hz, 1H), 7.75 (d, J=7.9 Hz, 1H), 7.59 (t, J=7.8 Hz, 1H), 7.18 (s, 2H), 6.89 (dd, J=7.9, 4.7 Hz, 1H), 2.19 (s, 3H), 2.08 (s, 3H), 1.75 (s, 6H). MS (ESI) m/z 503.3 [M+H]⁺

### Example 13. Synthesis of N-(3-(3-(9H-purin-6-yl)pyridin-2-ylamino)-4-methylphenyl)-2-(2-cyanopropan-2-yl)isonicotinamide (Compound I-11)

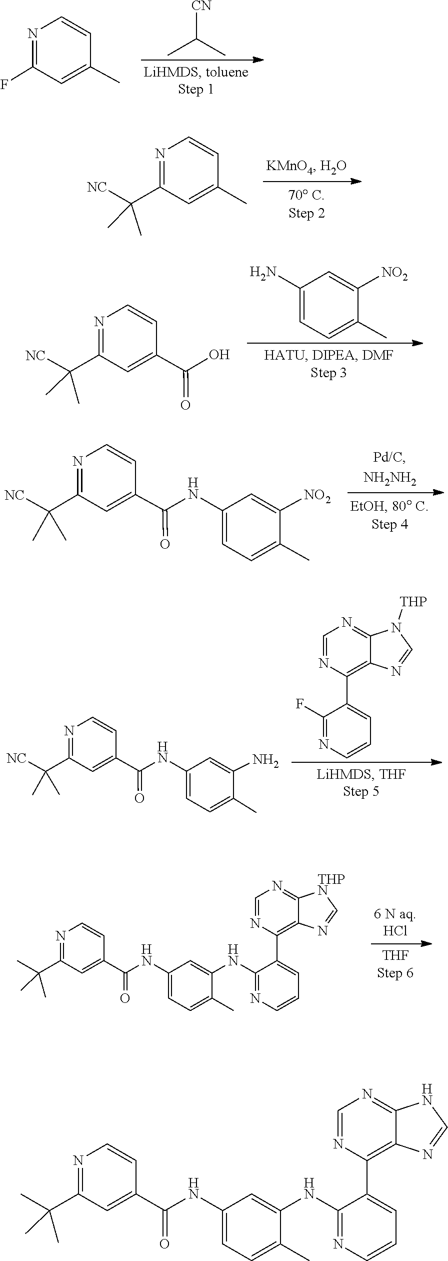

Step 1. To a solution of 2-fluoro-4-methylpyridine (1.0 g, 9.0 mmol) and isobutyronitrile (0.62 mg, 9.0 mmol) in toluene (15 mL) was added lithium hexamethyldisilazide (1.6 M in tetrahydrofuran, 8.4 mL, 13.5 mmol) at 0° C. slowly. The mixture was stirred at 20° C. for 12 h. The reaction was quenched with saturated ammonium chloride solution (20 mL) at 0° C. and extracted with ethyl acetate (50 mL×3). The combined organic layers were washed with brine (50 mL), dried over sodium sulfate, and filtered. The filtrate was concentrated under reduced pressure. The residue was purified by flash chromatography (petroleum ether/ethyl acetate=1/10) to afford 2-methyl-2-(4-methylpyridin-2-yl)propanenitrile (0.85 g, 5.31 mmol, 59%) as a yellow solid. MS (ESI) m/z 161.1 [M+H]⁺

Step 2. A solution of 2-methyl-2-(4-methylpyridin-2-yl)propanenitrile (850 mg, 5.31 mmol) and potassium permanganate (4.3 g, 27.2 mmol) in water (15 mL) was stirred at 70° C. for 24 h under argon. The reaction mixture was adjusted to pH-3.0 with 2N HCl. The mixture was diluted with ethyl acetate (50 mL), then the organic layer was washed with water (25 mL) and brine (25 mL), dried over sodium sulfate, filtered, and concentrated. The residue was purified by flash chromatography (dichloromethane/methanol=10/1) to afford 2-(2-cyanopropan-2-yl)isonicotinic acid (300 mg, 1.58 mmol, 30%) as a white solid. MS (ESI) m/z 191.1 [M+H]⁺

Step 3. A solution of 2-(2-cyanopropan-2-yl)isonicotinic acid (300 mg, 1.58 mmol), 4-methyl-3-nitroaniline (304 mg, 2.00 mmol), 1-[bis(dimethylamino)methylene]-1H-1,2,3-triazolo[4,5-b]pyridinium 3-oxid hexafluoro phosphate (1.14 g, 3.00 mmol) and N,N-diisopropylethylamine (516 mg, 4.00 mmol) in N,N-dimethylformamide (10 mL) was stirred at 20° C. for 1 h. The mixture was diluted with ethyl acetate (50 mL), washed with water (50 mL) and brine (50 mL), dried over sodium sulfate, filtered, and concentrated. The residue was purified by flash chromatography (ethyl acetate/petroleum ether=1/5) to get 2-(2-cyanopropan-2-yl)-N-(4-methyl-3-nitrophenyl)isonicotinamide (250 mg, 0.77 mmol, 49%) as a yellow solid. MS (ESI) m/z 325.2 [M+H]⁺

Step 4. A mixture of 2-(2-cyanopropan-2-yl)-N-(4-methyl-3-nitrophenyl)isonicotinamide (250 mg, 0.77 mmol), hydrazine hydrate (1.5 mL) and palladium (10% on activated carbon, 150 mg) in ethanol (50 mL) was stirred at 80° C. for 1 h. The reaction mixture was cooled, filtered, and concentrated. The residue was diluted with ethyl acetate (100 mL), washed with water (100 mL) and brine (60 mL), dried over sodium sulfate, filtered, and concentrated to give crude N-(3-amino-4-methylphenyl)-2-(2-cyanopropan-2-yl)isonicotinamide (200 mg, 0.68 mmol, 88%) as a yellow solid. MS (ESI) m/z 295.2 [M+H]⁺

Step 5. To a solution of N-(3-amino-4-methylphenyl)-2-(2-cyanopropan-2-yl)isonicotinamide (200 mg, 0.68 mmol) and 6-(2-fluoropyridin-3-yl)-9-(tetrahydro-2H-pyran-2-yl)-9H-purine (204 mg, 0.68 mmol) in tetrahydrofuran (10 mL) was added lithium hexamethyldisilazide (1.6 M in tetrahydrofuran, 2.1 mL, 3.4 mmol) at 0° C. slowly. The mixture was stirred at 20° C. for 5 h. The reaction was quenched with saturated ammonium chloride solution (20 mL) at 0° C. and extracted with ethyl acetate (50 mL×3). The combined organic layer was washed with brine (50 mL), dried over sodium sulfate, and filtered. The filtrate was concentrated under reduced pressure. The residue was purified by flash chromatography (petroleum ether/ethyl acetate=1/10) to give 2-(2-cyanopropan-2-yl)-N-(4-methyl-3-(3-(9-(tetrahydro-2H-pyran-2-yl)-9H-purin-6-yl)pyridin-2-ylamino)phenyl)isonicotinamide (120 mg, 0.21 mmol, 31%) as a yellow solid. MS (ESI) m/z 574.3 [M+H]⁺

Step 6. To a solution of 2-(2-cyanopropan-2-yl)-N-(4-methyl-3-(3-(9-(tetrahydro-2H-pyran-2-yl)-9H-purin-6-yl)pyridin-2-ylamino)phenyl)isonicotinamide (120 mg, 0.21 mmol) in tetrahydrofuran (21 mL) was added hydrochloric acid (6 N, 3.5 mL) at 20° C., and the mixture was stirred at 20° C. for 16 h. After completed, the reaction mixture was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-(3-(9H-purin-6-yl)pyridin-2-ylamino)-4-methylphenyl)-2-(2-cyanopropan-2-yl)isonicotinamide (25 mg, 0.05 mmol, 24%) as a yellow solid. ¹H NMR (400 MHZ, DMSO-d₆) δ 13.88 (s, 1H), 12.38 (s, 1H), 10.54 (s, 1H), 9.84 (d, J=6.8 Hz, 1H), 8.99 (s, 1H), 8.80-8.70 (m, 3H), 8.37 (dd, J=1.6 Hz, 4.8 Hz, 1H), 8.01 (s, 1H), 7.89 (dd, J=1.6 Hz, 4.8 Hz, 1H), 7.47-7.44 (m, 1H), 7.28 (d, J=8.4 Hz, 1H), 7.06-7.03 (m, 1H), 2.32 (s, 3H), 1.77 (s, 6H). MS (ESI) m/z 490.2 [M+H]⁺

### Example 14. Synthesis of 2-(2-(3-(3-(9H-purin-6-yl)pyridin-2-ylamino)-4-methylphenyl)-1H-benzo[d]imidazol-6-yl)-2-methylpropanenitrile (Compound I-12)

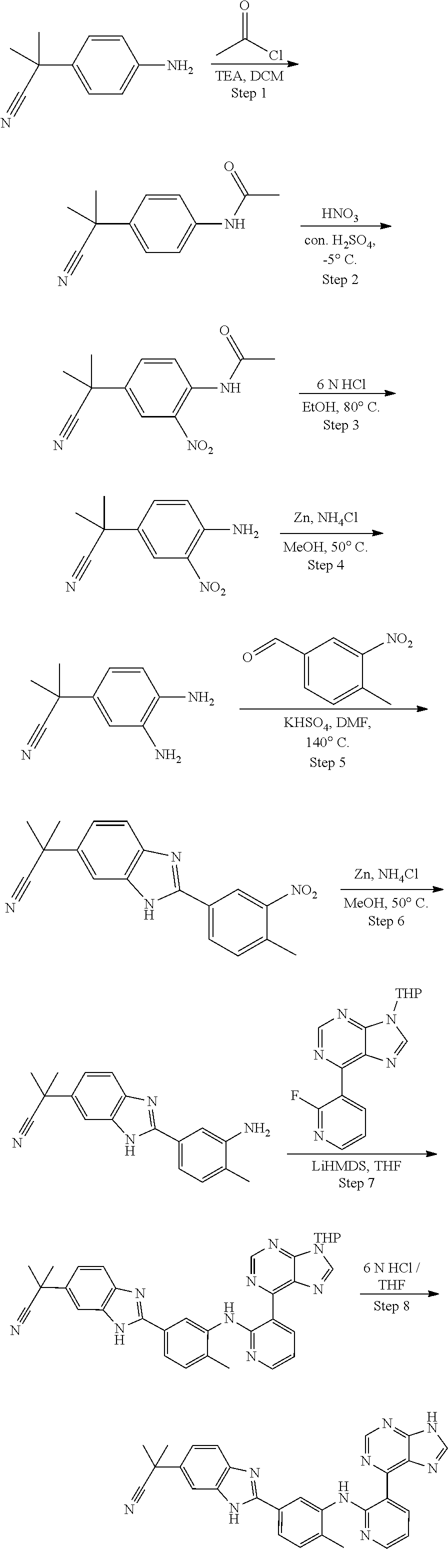

Step 1. To a mixture of acetyl chloride (590 mg, 7.5 mmol) in dichloromethane (15 mL) was added 2-(4-aminophenyl)-2-methylpropanenitrile (1.0 g, 6.25 mmol) and triethylamine (1.9 g 18.75 mmol) at room temperature. The reaction mixture was stirred at room temperature for 2 h. The reaction was diluted with ethyl acetate (20 mL), washed with saturated sodium bicarbonate aqueous solution (50 mL) and brine (40 mL), dried over sodium sulfate, filtered, and concentrated. The residue was purified by silica gel column chromatography, eluting with petroleum ether/ethyl acetate=4/1 to give N-(4-(2-cyanopropan-2-yl)phenyl)acetamide (1.1 g, 5.44 mmol, 87%) as a white solid. MS (ESI) m/z 203.0[M+H]⁺

Step 2. To a solution of N-(4-(2-cyanopropan-2-yl)phenyl)acetamide (1.1 g, 5.44 mol) in concentrated sulfuric acid (5 mL) at 0-10° C. was added concentrated nitric acid (60%, 460 mg, 4.35 mol). After stirring at −5° C. for 20 minutes, the reaction mixture was poured onto ice and the pH of the aqueous layer was adjusted to 7 with aqueous sodium hydroxide solution. The mixture was extracted with ethyl acetate, and the extracts were dried over sodium sulfate and concentrated to afford N-(4-(2-cyanopropan-2-yl)-2-nitrophenyl)acetamide (1 g, 4.04 mmol, 74%). MS (ESI) m/z 248.0[M+H]⁺

Step 3. To a solution of N-(4-(2-cyanopropan-2-yl)-2-nitrophenyl)acetamide (900 mg, 3.64 mmol) in ethanol (10 mL) was added hydrochloric acid (6 N, 2 mL). The mixture was stirred at 80° C. for 6 h. The reaction mixture was cooled, and a saturated solution of sodium bicarbonate was added to adjust pH to 7-8. The resulting mixture was extracted with 2-methyltetrahydrofuran (50 mL×2). The combined organic layers were washed with brine, dried over sodium sulfate, filtered, and concentrated. The residue was purified by reversed-phase chromatography (water:acetonitrile=2:1) to afford 2-(4-amino-3-nitrophenyl)-2-methylpropanenitrile (720 mg, 3.51 mmol, 96%) as a white solid. MS (ESI) m/z 206.0[M+H]⁺

Step 4. To a mixture of 2-(4-amino-3-nitrophenyl)-2-methylpropanenitrile (720 mg, 3.51 mmol) and ammonium chloride (1.9 g, 35.1 mmol) in methanol (40 mL) and water (8 mL) was added zinc powder (1.14 g, 17.6 mmol). The mixture was stirred at 50° C. for 2 h. The mixture was filtered, and the filtrate was concentrated. The residue was diluted with water (30 mL), extracted with dichloromethane (30 mL×3). The combined organic layer was washed with water (30 mL) and brine (30 mL), dried over sodium sulfate, filtered, and concentrated in vacuo to afford 2-(3,4-diaminophenyl)-2-methylpropanenitrile (420 mg, 2.0 mmol, 68%) as a yellow solid. MS (ESI) m/z 176.1 [M+H]⁺

Step 5. A solution of 4-methyl-3-nitrobenzaldehyde (397 mg, 2.4 mmol) and sodium bisulfate (980 mg, 7.2 mmol) in dimethylformamide (10 mL) was refluxed for 1 h. Then a solution of 2-(3,4-diaminophenyl)-2-methylpropanenitrile (420 mg, 2.4 mmol) in N,N-dimethylformamide (10 mL) was added dropwise and the mixture was refluxed for an additional 3 h. The mixture was cooled to room temperature and poured into ice water. The organic layer was washed with brine and dried over sodium sulfate, then evaporated to dryness. The residue obtained was triturated with petroleum ether in order to eliminate impurities and the solid was collected by filtration to give 2-methyl-2-(2-(4-methyl-3-nitrophenyl)-1H-benzo[d]imidazol-6-yl)propanenitrile (240 mg, 0.75 mmol, 31%) as a brown solid. MS (ESI) m/z 321.0 [M+H]⁺

Step 6. To a mixture of 2-methyl-2-(2-(4-methyl-3-nitrophenyl)-1H-benzo[d]imidazol-6-yl)propanenitrile (240 mg, 0.75 mmol) and ammonium chloride (401 mg, 7.5 mmol) in methanol (40 mL) and water (8 mL) was added zinc powder (244 mg, 3.75 mmol), the mixture was stirred at 50° C. for 2 h. The mixture was filtered, and the filtrate was concentrated. The residue was diluted with water (30 mL), extracted with dichloromethane (30 mL×3). The combined organic layer was washed with water (30 mL) and brine (30 mL), dried over sodium sulfate, filtered, and concentrated in vacuo to afford 2-(2-(3-amino-4-methylphenyl)-1H-benzo[d]imidazol-6-yl)-2-methylpropanenitrile as a yellow solid. (70 mg, 0.24 mmol, 32%). MS (ESI) m/z 291.0 [M+H]⁺

Step 7. Lithium hexamethyldisilazide (1.6 M in tetrahydrofuran, 0.9 mL, 1.44 mmol) was added to a mixture of 2-(2-(3-amino-4-methylphenyl)-1H-benzo[d]imidazol-6-yl)-2-methylpropanenitrile (70 mg, 0.24 mmol) and 6-(2-fluoropyridin-3-yl)-9-(tetrahydro-2H-pyran-2-yl)-9H-purine (72 mg, 0.24 mmol) in tetrahydrofuran (20 mL) at 0° C. under nitrogen. The reaction mixture was stirred at room temperature for 2 h. The reaction was diluted with ethyl acetate (200 mL), washed with saturated sodium bicarbonate aqueous solution (50 mL) and brine (40 mL), dried over sodium sulfate, filtered, and concentrated to give 2-methyl-2-(2-(4-methyl-3-((3-(9-(tetrahydro-2H-pyran-2-yl)-9H-purin-6-yl)pyridin-2-yl)amino)phenyl)-1H-benzo[d]imidazol-6-yl)propanenitrile (90 mg, 0.16 mmol, 66%) as a yellow solid. MS (ESI) m/z 570 [M+H]⁺

Step 8. A mixture of 2-methyl-2-(2-(4-methyl-3-((3-(9-(tetrahydro-2H-pyran-2-yl)-9H-purin-6-yl) pyridin-2-yl)amino)phenyl)-1H-benzo[d]imidazol-6-yl)propanenitrile (90 mg, 0.16 mmol) in tetrahydrofuran (5 mL) was added hydrochloric acid (6 N, 6 mL) at room temperature. Then the reaction mixture was stirred at room temperature for 16 h. The reaction was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 2-(2-(3-((3-(9H-purin-6-yl)pyridin-2-yl)amino)-4-methylphenyl)-1H-benzo[d]imidazol-6-yl)-2-methylpropanenitrile (25 mg, 0.0515 mmol, 32%) as a yellow solid. ¹H NMR (500 MHz, DMSO-d₆) δ 13.85 (s, 1H), 12.88 (s, 1H), 12.41 (s, 1H), 9.83 (s, 1H), 9.10 (d, J=8.7 Hz, 2H), 8.73 (s, 1H), 8.42 (d, J=2.8 Hz, 1H), 7.83-7.66 (m, 2H), 7.65-7.25 (m, 3H), 7.09 (dd, J=7.8, 4.6 Hz, 1H), 2.53 (s, 3H), 1.77 (s, 6H). MS (ESI) m/z 486 [M+H]⁺

### Example 15. Synthesis of N-(3-(3-(9H-purin-6-yl)pyridin-2-ylamino)-4-methylphenyl)-2-(1-cyanocyclopropyl)isonicotinamide (Compound I-13)

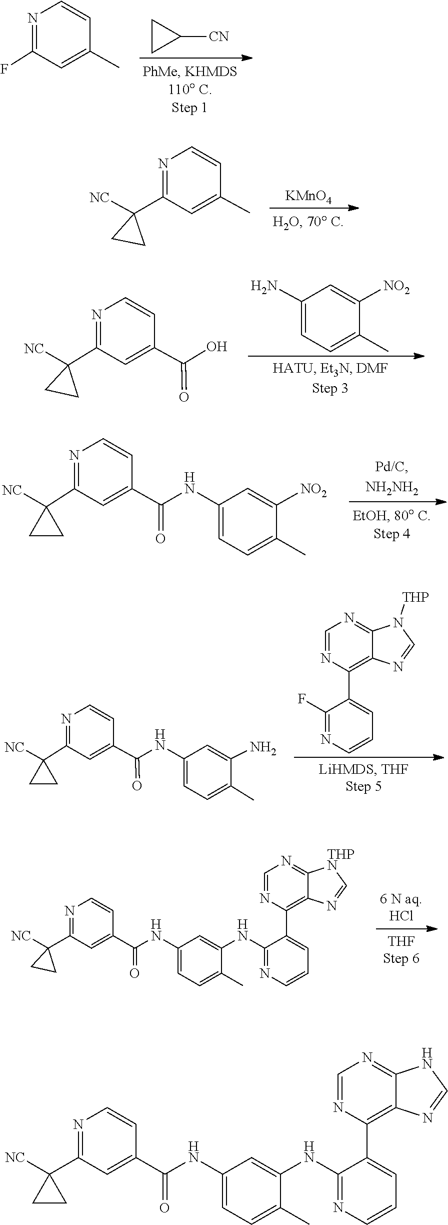

Step 1. To a mixture of 2-fluoro-4-methylpyridine (1.0 g, 4.50 mmol) in toluene (15 mL) was added cyclopropanecarbonitrile (724 mg, 5.54 mmol) and potassiumhexamethyldisilazide (13.5 mL, 13.5 mmol) at 25° C. Then the reaction mixture was stirred at 110° C. for 2 h. The reaction was diluted with ethyl acetate (50 mL×3), washed with ammonium chloride aqueous solution (50 mL) and brine (40 mL), dried over sodium sulfate, filtered, and concentrated. The residue was purified by silica gel column chromatography, eluting with petroleum ether/ethyl acetate=4/1 to give 1-(4-methylpyridin-2-yl)cyclopropane-1-carbonitrile (600 mg, 3.80 mmol, 84%) as a white solid. MS (ESI) m/z 159.1[M+H]⁺

Step 2. A mixture of 1-(4-methylpyridin-2-yl)cyclopropane-1-carbonitrile (600 mg, 3.80 mmol) and potassium permanganate (1.2 g, 7.6 mmol) in water (15 mL) was stirred at 70° C. for 3 days. After cooling to room temperature, the mixture was diluted with ethyl acetate (40 mL) and filtered through t celite. The filtrate was concentrated and dried to give (1-cyanocyclopropyl)isonicotinic acid (500 mg, crude) as a yellow solid. MS (ESI) m/z 189.7 [M+H]⁺

Step 3. To a mixture of (1-cyanocyclopropyl)isonicotinic acid (500 mg, 2.66 mmol) in N,N-dimethylformamide (15 mL) was added 4-methyl-3-nitroaniline (486 mg, 3.2 mmol), triethylamine (806 mg, 7.98 mmol) and 1-[bis(dimethylamino)methylene]-1H-1,2,3-triazolo[4,5-b]pyridinium 3-oxid hexafluorophosphate (1.22 g, 3.2 mmol). Then the reaction mixture was stirred at room temperature for 1 h. The reaction was diluted with ethyl acetate (50 mL×3), washed with saturated sodium bicarbonate aqueous solution (50 mL) and brine (40 mL), dried over sodium sulfate, filtered, and concentrated. The residue was purified by silica gel column chromatography, eluting with petroleum ether/ethyl acetate=3/1 to give 2-(1-cyanocyclopropyl)-N-(4-methyl-3-nitrophenyl)isonicotinamide (300 mg, 0.93 mmol, 35%) as a yellow solid. MS (ESI) m/z 323.1 [M+H]⁺

Step 4. Palladium (10% Pd on activated carbon, 50 mg) was added to a mixture of 2-(1-cyanocyclopropyl)-N-(4-methyl-3-nitrophenyl)isonicotinamide (300 mg, 0.93 mmol) and hydrazine hydrate (1.0 g, 20 mmol) in ethanol (5 mL) at room temperature. The mixture was stirred at 80° C. for 1 h. After cooling to room temperature, the mixture was diluted with ethyl acetate (40 mL) and filtered through celite. The filtrate was concentrated and dried to give N-(3-amino-4-methylphenyl)-2-(1-cyanocyclopropyl)isonicotinamide (150 mg, 0.51 mmol, 55%) as yellow solid. MS (ESI) m/z 293.1 [M+H]⁺

Step 5. Lithium hexamethyldisilazide (1.6 M in tetrahydrofuran, 0.43 mL, 0.68 mmol) was added to a mixture of N-(3-amino-4-methylphenyl)-2-(1-cyanocyclopropyl)isonicotinamide (100 mg, 0.34 mmol) and 6-(2-fluoro pyridin-3-yl)-9-(tetrahydro-2H-pyran-2-yl)-9H-purine (103 mg, 0.34 mmol) in tetrahydrofuran (5 mL) at 0° C. under nitrogen. Then the reaction mixture was stirred at 25° C. for 2 h. The reaction was diluted with ethyl acetate (20 mL), washed with saturated sodium bicarbonate aqueous solution (20 mL) and brine (40 mL), dried over sodium sulfate, filtered, and concentrated to give 2-(1-cyanocyclopropyl)-N-(4-methyl-3-((3-(9-(tetrahydro-2H-pyran-2-yl)-9H-purin-6-yl)pyridin-2-yl)amino)phenyl)isonicotinamide (100 mg, 0.17 mmol, 50%) as a yellow solid. MS (ESI) m/z 572.1 [M+H]⁺

Step 6. To a mixture of 2-(1-cyanocyclopropyl)-N-(4-methyl-3-((3-(9-(tetrahydro-2H-pyran-2-yl)-9H-purin-6-yl)pyridin-2-yl)amino)phenyl)isonicotinamide (100 mg, 0.17 mmol) in tetrahydrofuran (5 mL) was added hydrochloric acid (6 N, 5 mL) at room temperature. Then the reaction mixture was stirred at room temperature for 16 h. The mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl) pyridin-2-yl)amino)-4-methylphenyl)-2-(trifluoromethyl)-isonicotinamide (7.5 mg, 0.015 mmol, 9%) as a yellow solid. ¹H NMR (400 MHZ, DMSO-d₆) δ 12.45 (s, 1H), 10.56 (s, 1H), 9.85 (d, J=15.0 Hz, 1H), 9.04 (s, 1H), 8.77-8.58 (m, 4H), 8.35 (d, J=3.5 Hz, 1H), 7.93 (s, 1H), 7.82 (d, J=5.1 Hz, 1H), 7.46 (d, J=8.1 Hz, 1H), 7.26 (d, J=8.2 Hz, 1H), 7.04 (dd, J=7.8, 4.8 Hz, 1H), 2.46 (s, 3H), 1.89 (dd, J=7.8, 4.4 Hz, 2H), 1.78 (dd, J=7.8, 4.5 Hz, 2H). MS (ESI) m/z 488.1 [M+H]⁺

### Example 16. Synthesis of N-(3-(3-(9H-purin-6-yl)pyridin-2-ylamino)-4-methylphenyl)-2-(4,4-difluorocyclohexyl)acetamide (Compound I-14)

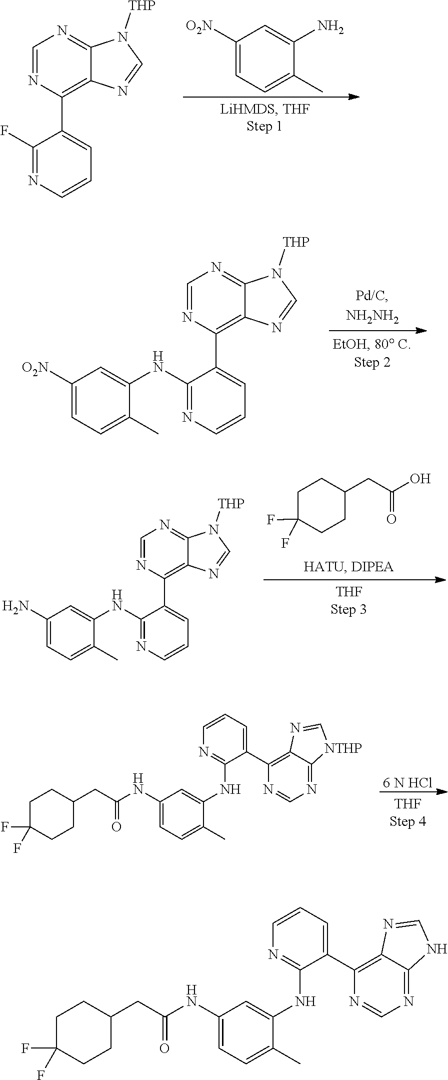

Step 1. To a mixture of 6-(2-fluoropyridin-3-yl)-9-(tetrahydro-2H-pyran-2-yl)-9H-purine (1 g, 3.34 mmol) and 2-methyl-5-nitroaniline (510 mg, 3.34 mmol) in tetrahydrofuran (25 mL) was added dropwise lithium hexamethyldisilazide (1.6 M in tetrahydrofuran, 10.4 mL, 16.6 mmol) at 0° C. The mixture was stirred at room temperature for 2 h. The mixture was cooled to 0° C., and saturated ammonium chloride solution (3 mL) was added slowly. The mixture was extracted with dichloromethane (50 mL×5), the organic layer was concentrated and purified by flash chromatography (methanol/dichloromethane=4/96) to give N-(2-methyl-5-nitrophenyl)-3-(9-(tetrahydro-2H-pyran-2-yl)-9H-purin-6-yl)pyridin-2-amine (0.7 g, 1.62 mmol, 49%) as a yellow solid. MS (ESI) m/z 432.0 [M+H]⁺

Step 2. To a solution of N-(2-methyl-5-nitrophenyl)-3-(9-(tetrahydro-2H-pyran-2-yl)-9H-purin-6-yl)pyridin-2-amine (0.7 g, 1.62 mmol) in ethanol (25 mL) was added palladium (10% Pd on activated carbon, 500 mg) and hydrazine hydrate (2 mL). The mixture was stirred at 80° C. under nitrogen for 1 h. The mixture was cooled to room temperature and filtered. The filtrate was dried over sodium sulfate and concentrated to give 6-methyl-N1-(3-(9-(tetrahydro-2H-pyran-2-yl)-9H-purin-6-yl)pyridin-2-yl)benzene-1,3-diamine (0.5 g, 1.25 mmol, 77%) as a yellow solid. MS (ESI) m/z 402.0 [M+H]⁺

Step 3. A mixture of 2-(4,4-difluorocyclohexyl)acetic acid (50 mg, 0.28 mmol), diisopropylethylamine (0.14 mL, 0.84 mmol) and 1-[bis(dimethylamino)methylene]-1H-1,2,3-triazolo[4,5-b]pyridinium 3-oxid hexafluorophosphate (160 mg, 0.42 mmol) in dichloromethane (8 mL) was stirred at room temperature for 30 min, then 6-methyl-N1-(3-(9-(tetrahydro-2H-pyran-2-yl)-9H-purin-6-yl)pyridin-2-yl)benzene-1,3-diamine (113 mg, 0.28 mmol) was added. The mixture was stirred at room temperature for 1 h. The mixture was concentrated and purified by flash chromatography (methanol/dichloromethane=4/96) to give 2-(4,4-difluorocyclohexyl)-N-(4-methyl-3-((3-(9-(tetrahydro-2H-pyran-2-yl)-9H-purin-6-yl)pyridin-2-yl)amino)phenyl)-acetamide (0.10 g, 0.18 mmol, 63%) as a yellow solid. MS (ESI) m/z 562.0 [M+H]⁺

Step 4. To a solution of 2-(4,4-difluorocyclohexyl)-N-(4-methyl-3-((3-(9-(tetrahydro-2H-pyran-2-yl)-9H-purin-6-yl)pyridin-2-yl)amino)phenyl)acetamide (100 mg, 0.18 mmol) in tetrahydrofuran (8 mL) was added 6 N hydrochloric acid (2 mL). The mixture was stirred at room temperature for 1 h. The mixture was concentrated, washed with saturated sodium bicarbonate solution and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-2-(4,4-difluoro-cyclohexyl)acetamide (21.0 mg, 0.03 mmol, 25%) as a yellow solid. ¹H NMR (400 MHZ, DMSO-d₆) δ 12.25 (s, 1H), 9.82-9.76 (m, 2H), 9.06 (s, 1H), δ 8.70 (s, 1H), 8.43 (s, 1H), 8.35-8.33 (m, 1H), 7.41-7.38 (m, 2H), 7.16-7.14 (m, 1H), 7.04-7.01 (m, 1H), 2.39 (s, 3H), 2.27 (d, J=7.2 Hz, 2H), 2.00-1.78 (m, 7H), 1.27-1.24 (m, 2H). MS (ESI) m/z 478.2 [M+H]⁺

### Example 17. Synthesis of 2-(3-((5-((3-(9H-purin-6-yl)pyridin-2-yl)amino)-6-methylpyridin-3-yl)ethynyl)phenyl)-2-methylpropanenitrile (Compound I-15)

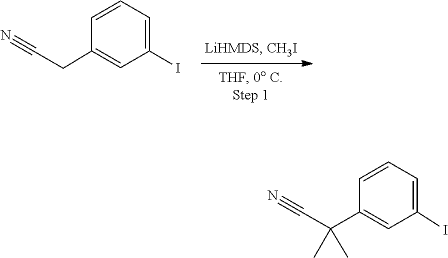

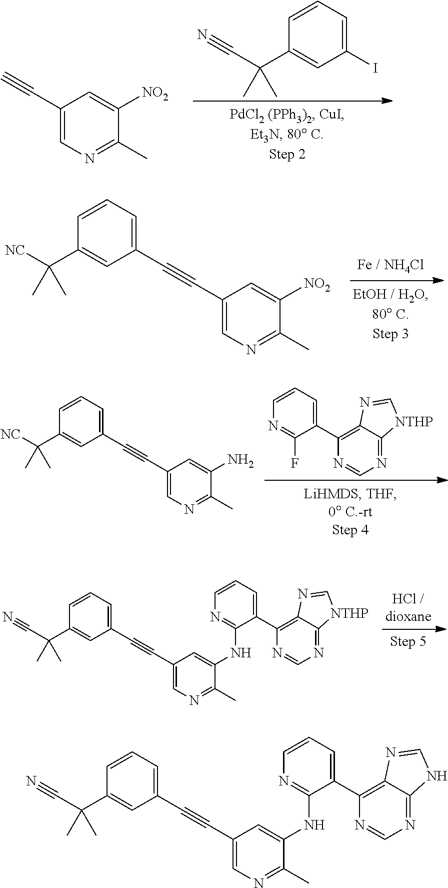

Step 1. Lithium hexamethyldisilazide (1.6 M in tetrahydrofuran, 10.3 mL, 16.5 mmol) was added slowly to a solution of 2-(3-iodophenyl)acetonitrile (2.0 g, 8.23 mmol) in tetrahydrofuran (15 mL) at 0° C. After addition was completed, the reaction mixture was stirred at 0° C. for a further 20 minutes. Methyl iodide (5.8 g, 41.2 mmol, 2.5 mL) was then added to the reaction mixture and the reaction was stirred at room temperature for 1 h. The crude mixture was quenched with water and extracted with ethyl acetate. The organic phase was washed with brine, dried over sodium sulfate, filtered, and concentrated in vacuo. The residue was purified by flash column chromatography (petroleum ether/ethyl acetate=10/1) to afford 2-(3-iodophenyl)-2-methylpropanenitrile (2.2 g, 8.86 mmol, 91%) as a colorless oil. MS (ESI) m/z 272.0 [M+H]⁺

Step 2. A mixture of 5-ethynyl-2-methyl-3-nitropyridine (300 mg, 1.85 mmol), 2-(3-iodophenyl)-2-methyl propanenitrile (502 mg, 1.85 mmol), bis(triphenylphosphine)palladium(II) chloride (130 mg, 0.19 mmol) and cuprous iodide (70 mg, 0.37 mmol) in triethylamine (5 mL) was stirred at 80° C. for 4 h under nitrogen. The reaction was filtered through celite and washed with ethyl acetate. The filtrate was washed with brine (40 mL), dried over sodium sulfate, filtered, and concentrated. The residue was purified by flash chromatography (petroleum ether/ethyl acetate=10/1) to give 2-methyl-2-(3-((6-methyl-5-nitropyridin-3-yl)ethynyl)phenyl)-propanenitrile (400 mg, 1.31 mmol, 71%) as a yellow solid. MS (ESI) m/z 306.1 [M+H]⁺

Step 3. A mixture of 2-methyl-2-(3-((6-methyl-5-nitropyridin-3-yl)ethynyl)phenyl)propanenitrile (200 mg, 0.66 mmol), iron (147 mg, 2.62 mmol) and ammonium chloride (278 mg, 5.25 mmol) in ethanol (24 mL) and water (16 mL) was stirred at 80° C. for 1 h. After cooling to room temperature, the reaction was filtered through celite and washed with ethyl acetate. The filtrate was washed with saturated sodium bicarbonate aqueous solution (50 mL) and brine (40 mL), dried over sodium sulfate, filtered, and concentrated. The residue was purified by flash chromatography (dichloromethane/methanol=10/1) to give 2-(3-((5-amino-6-methylpyridin-3-yl)ethynyl)phenyl)-2-methylpropanenitrile (160 mg, 0.58 mmol, 89%) as a yellow solid. MS (ESI) m/z 276.1 [M+H]⁺

Step 4. To a mixture of 2-(3-((5-amino-6-methylpyridin-3-yl)ethynyl)phenyl)-2-methylpropanenitrile (140 mg, 0.51 mmol) in tetrahydrofuran (5 mL) was added 6-(2-fluoropyridin-3-yl)-9-(tetrahydro-2H-pyran-2-yl)-9H-purine (152 mg, 0.51 mmol) at 0° C. under nitrogen, followed by lithium hexamethyldisilazide (1.6 M in tetrahydrofuran, 1.9 mL, 3.05 mmol). Then the reaction mixture was stirred at 0° C. to room temperature for 2 h. The reaction was diluted and extracted with ethyl acetate (100 mL), washed with brine (40 mL), dried over sodium sulfate, filtered, and concentrated. The residue was purified by flash chromatography (dichloromethane/methanol=10/1) to give 2-methyl-2-(3-((6-methyl-5-((3-(9-(tetrahydro-2H-pyran-2-yl)-9H-purin-6-yl)pyridin-2-yl)amino)pyridin-3-yl)ethynyl)phenyl)propanenitrile (160 mg, 0.29 mmol, 57%) as a white solid. MS (ESI) m/z 554.8 [M+H]⁺

Step 5. To a mixture of 2-methyl-2-(3-((6-methyl-5-((3-(9-(tetrahydro-2H-pyran-2-yl)-9H-purin-6-yl) pyridin-2-yl)amino)pyridin-3-yl)ethynyl)phenyl)propanenitrile (150 mg, 0.27 mmol) in dioxane (3 mL) was added hydrogen chloride solution (4 M in dioxane, 5 mL) at room temperature. Then the reaction mixture was stirred at room temperature for 2 h. The reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 2-(3-((5-((3-(9H-purin-6-yl)pyridin-2-yl)amino)-6-methylpyridin-3-yl)ethynyl)phenyl)-2-methylpropanenitrile (34.4 mg, 0.07 mmol, 27%) as a yellow solid. ¹H NMR (400 MHZ, DMSO-d₆) δ 1.74 (s, 6H), 2.74 (s, 3H), 7.11-7.14 (m, 1H), 7.52 (t, J=7.6 Hz, 1H), 7.60 (d, J=7.6 Hz, 2H), 7.75 (s, 1H), 8.29 (d, J=1.6 Hz, 1H), 8.44 (dd, J₁=1.6 Hz, J₂=4.8 Hz, 1H), 8.73 (s, 1H), 9.07 (t, J=4.4 Hz, 2H), 9.85 (d, J=5.6 Hz, 1H), 12.72 (s, 1H), 13.88 (s, 1H). MS (ESI) m/z 470.7 [M+H]⁺

### Example 18. Synthesis of N-(5-(3-(9H-purin-6-yl)pyridin-2-ylamino)-6-methylpyridin-3-yl)-4-(trifluoromethyl)picolinamide (Compound I-16)

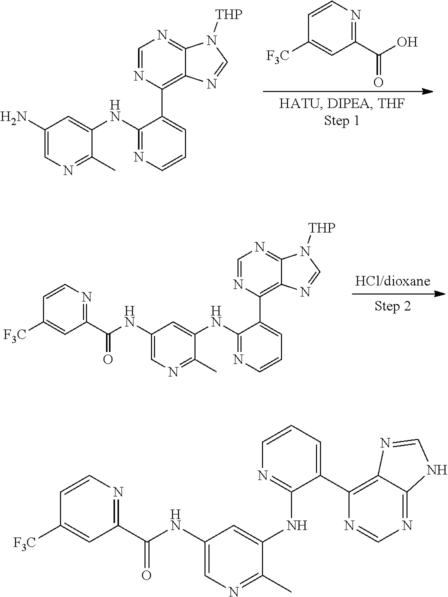

Step 1. A solution of 2-methyl-N3-(3-(9-(tetrahydro-2H-pyran-2-yl)-9H-purin-6-yl)pyridin-2-yl) pyridine-3,5-diamine (201 mg, 0.5 mmol), 4-(trifluoromethyl)picolinic acid (124 mg, 0.65 mmol), O-(7-azabenzotriazol-1-yl)-N,N,N′,N′-tetramethyluroniun hexafluorophosphate (372 mg, 0.98 mmol) and N,N-diisopropylethyl amine (258 mg, 2.0 mmol) in tetrahydrofuran (20 mL) was stirred at 20° C. for 18 h. The mixture was concentrated and purified by flash chromatography (methanol/dichloromethane=10:100) to afford N-(6-methyl-5-(3-(9-(tetrahydro-2H-pyran-2-yl)-9H-purin-6-yl) pyridin-2-ylamino)pyridin-3-yl)-4-(triforomethyl)-picolinamide (127 mg, 0.22 mmol, 44%) as a yellow solid. MS (ESI) m/z 576.2 [M+H]⁺

Step 2. To a solution of N-(6-methyl-5-(3-(9-(tetrahydro-2H-pyran-2-yl)-9H-purin-6-yl)pyridin-2-ylamino)pyridin-3-yl)-4-(trifluoromethyl)picolinamide (127 mg, 0.22 mmol) in dioxane (10 mL) was added hydrogen chloride solution (4 M in dioxane, 2 mL) at 20° C., and the mixture was stirred at 20° C. for 1 h. After completed, the reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(5-(3-(9H-purin-6-yl)pyridin-2-ylamino)-6-methylpyridin-3-yl)-4-(trifluoromethyl)picolinamide (17.1 mg, 0.035 mmol, 16%) as a yellow solid. ¹H NMR (400 MHZ, DMSO-d₆) δ 13.86 (s, 1H), 12.57 (s, 1H), 10.96 (s, 1H), 9.85 (s, 1H), 9.34 (d, J=2.2 Hz, 1H), 9.11 (s, 1H), 9.06 (d, J=5.1 Hz, 1H), 8.74 (s, 1H), 8.66 (d, J=2.3 Hz, 1H), 8.41 (dd, J=4.7, 1.9 Hz, 1H), 8.38 (s, 1H), 8.11 (d, J=5.0 Hz, 1H), 7.12 (dd, J=7.9, 4.7 Hz, 1H), 2.69 (s, 3H). MS (ESI) m/z 492.1 [M+H]⁺

### Example 19. Synthesis of N-(5-(3-(9H-purin-6-yl)pyridin-2-ylamino)-6-methylpyridin-3-yl)-2-(trifluoromethyl)isonicotinamide (Compound I-17)

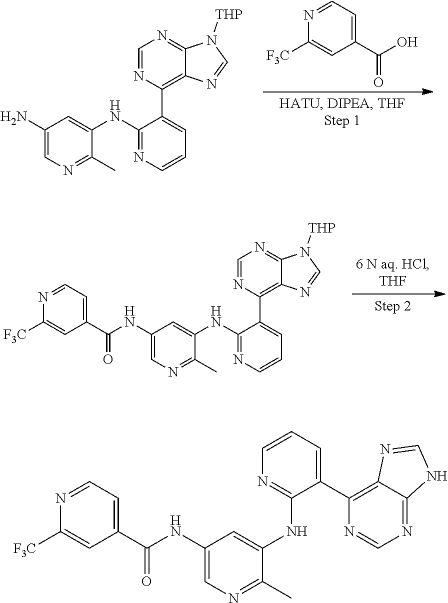

Step 1. A solution of 2-methyl-N3-(3-(9-(tetrahydro-2H-pyran-2-yl)-9H-purin-6-yl)pyridin-2-yl)pyridine-3,5-diamine (201 mg, 0.5 mmol), 2-(trifluoromethyl)isonicotinic acid (97 mg, 0.5 mmol), 1-[bis(dimethylamino)methylene]-1H-1,2,3-triazolo[4,5-b]pyridinium 3-oxid hexafluorophosphate (285 mg, 0.75 mmol) and N,N-diisopropylethylamine (194 mg, 1.5 mmol) in N,N-dimethylformamide (3 mL) was stirred at 20° C. for 1 h. The reaction mixture was diluted with ethyl acetate (50 mL) and washed with water (20 mL×2), and brine (30 mL), dried over sodium sulfate, filtered, and concentrated. The residue was purified by flash chromatography (methanol/dichloromethane=10/100) to get N-(6-methyl-5-(3-(9-(tetrahydro-2H-pyran-2-yl)-9H-purin-6-yl)pyridin-2-ylamino)pyridin-3-yl)-2-(trifluoromethyl)isonicotinamide (84 mg, 0.15 mmol, 30%) as a red solid. MS (ESI) m/z 576.2 [M+H]⁺

Step 2. To a solution of N-(6-methyl-5-(3-(9-(tetrahydro-2H-pyran-2-yl)-9H-purin-6-yl)pyridin-2-ylamino)pyridin-3-yl)-2-(trifluoromethyl)isonicotinamide (84 mg, 0.15 mmol) in tetrahydrofuran (6 mL) was added 6 N hydrochloric acid (1 mL) at 20° C. The mixture was stirred at 20° C. for 3 h. After completed, the reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(5-(3-(9H-purin-6-yl)pyridin-2-ylamino)-6-methylpyridin-3-yl)-2-(trifluoromethyl)isonicotinamide (25.0 mg, 0.05 mmol, 33%) as a yellow solid. ¹H NMR (500 MHZ, DMSO-d₆) δ 13.89 (s, 1H), 12.64 (s, 1H), 10.83 (s, 1H), 9.88 (s, 1H), 9.23 (s, 1H), 9.12 (s, 1H), 9.01 (d, J=4.8 Hz, 1H), 8.74 (s, 1H), 8.61 (s, 1H), 8.41 (d, J=4.7 Hz, 2H), 8.24 (d, J=4.4 Hz, 1H), 7.12 (dd, J=7.8, 4.7 Hz, 1H), 2.71 (s, 3H). MS (ESI) m/z 492.1 [M+H]⁺

### Example 20. Synthesis of N-(5-(3-(9H-purin-6-yl)pyridin-2-ylamino)-6-methylpyridin-3-yl)-5-(trifluoromethyl)nicotinamide (Compound I-18)

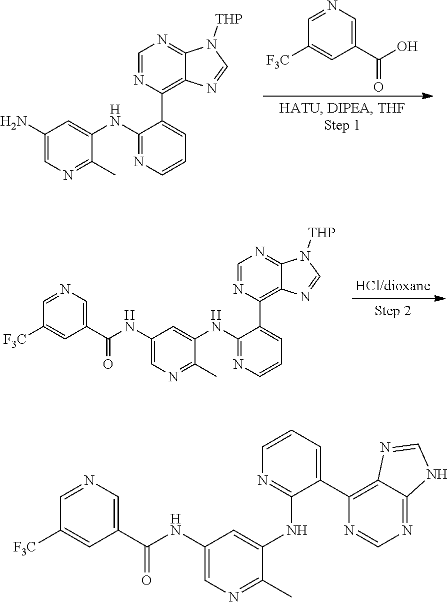

Step 1. A solution of 2-methyl-N3-(3-(9-(tetrahydro-2H-pyran-2-yl)-9H-purin-6-yl)pyridin-2-yl) pyridine-3,5-diamine (250 mg, 0.62 mmol), 5-(trifluoromethyl)nicotinic acid (155 mg, 0.81 mmol), O-(7-azabenzotriazol-1-yl)-N,N,N′,N′-tetramethyluroniun hexafluorophosphate (460 mg, 1.21 mmol) and N,N-diisopropylethylamine (320 mg, 2.48 mmol) in tetrahydrofuran (30 mL) was stirred at 20° C. for 18 h. The mixture was concentrated and purified by flash chromatography (methanol/dichloromethane=10/100) to afford N-(6-methyl-5-(3-(9-(tetrahydro-2H-pyran-2-yl)-9H-purin-6-yl)pyridin-2-ylamino)pyridin-3-yl)-5-(trifluoromethyl)-nicotinamide (147 mg, 0.26 mmol, 42%) as a yellow solid. MS (ESI) m/z 575.8 [M+H]⁺

Step 2. To a solution of N-(6-methyl-5-(3-(9-(tetrahydro-2H-pyran-2-yl)-9H-purin-6-yl)pyridin-2-ylamino)pyridin-3-yl)-5-(trifluoromethyl)nicotinamide (147 mg, 0.26 mmol) in dioxane (10 mL) was added hydrogen chloride solution (4 M in dioxane, 2 mL) at 20° C., and the mixture was stirred at 20° C. for 1 h. After completed, the reaction mixture was concentrated was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(5-(3-(9H-purin-6-yl)pyridin-2-ylamino)-6-methylpyridin-3-yl)-5-(trifluoromethyl)nicotinamide (52.4 mg, 0.11 mmol, 42%) as a yellow solid. ¹H NMR (400 MHZ, DMSO-d₆) δ 13.89 (s, 1H), 12.61 (s, 1H), 10.77 (s, 1H), 9.86 (s, 1H), 9.42 (s, 1H), 9.21 (d, J=11.4 Hz, 2H), 9.12 (s, 1H), 8.74 (s, 2H), 8.61 (d, J=1.8 Hz, 1H), 8.41 (d, J=3.0 Hz, 1H), 7.12 (dd, J=7.8, 4.6 Hz, 1H), 2.71 (s, 3H). MS (ESI) m/z 492.1 [M+H]⁺

### Example 21. Synthesis of N-(3-((3-(9H-purin-6-yl)pyridin-2-yl)amino)-4-methylphenyl)-2-cyclopropylisonicotinamide (Compound I-19)

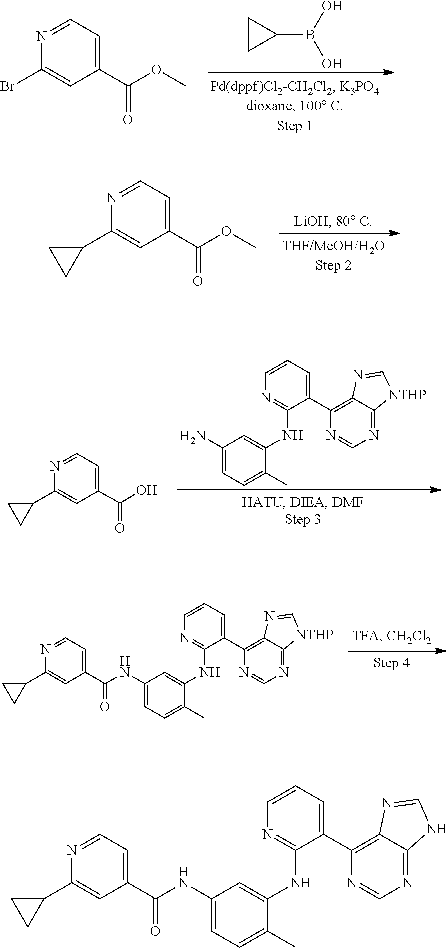

Step 1. A solution of methyl 2-bromoisonicotinate (2.0 g, 9.26 mmol), cyclopropylboronic acid (3.18 g, 37.0 mmol), 1,1′-bis(diphenylphosphino) ferrocene-palladium(II)dichloride dichloromethane complex (756 mg, 0.926 mmol) and potassium phosphate tribasic (5.89 g, 27.8 mmol) in dioxane (20 mL) was stirred at 100° C. under nitrogen atmosphere for 3 h. The mixture was filtered and concentrated. The crude residue was purified by flash chromatography (dichloromethane/methanol=19/1) to give methyl 2-cyclopropylisonicotinate (1.5 g, 8.4 mmol, 93.7%) as a brown oil. MS (ESI) m/z 178.1 [M+H]⁺

Step 2. A mixture of methyl 2-cyclopropylisonicotinate (1.5 g, 8.47 mmol) and lithium hydroxide (1.01 g, 42.4 mmol) in tetrahydrofuran (15 mL), water (5 mL) and methanol (5 mL) was stirred at 80° C. for 20 min. The reaction mixture was adjusted to pH=3.0 with hydrochloric acid (6 N). The resulting white precipitate was collected by filtration and washed with water (20 mL) to give 2-cyclopropylisonicotinic acid (1.0 g, 6.1 mmol, 73%) as a white solid. MS (ESI) m/z 164.1 [M+H]⁺

Step 3. A solution of 2-cyclopropylisonicotinic acid (60 mg, 0.37 mmol), 6-methyl-N1-(3-(9-(tetrahydro-2H-pyran-2-yl)-9H-purin-6-yl)pyridin-2-yl)benzene-1,3-diamine (100 mg, 0.37 mmol), 1-[bis(dimethylamino)methylene]-1H-1,2,3-triazolo[4,5-b]pyridinium 3-oxid hexafluorophosphate (155 mg, 0.41 mmol) and N,N-diisopropyl ethylamine (95 mg, 0.74 mmol) in tetrahydrofuran (5.0 mL) was stirred at room temperature for 2 h. The mixture was concentrated and the crude residue was purified by flash chromatography (100% ethyl acetate) to give 2-cyclopropyl-N-(4-methyl-3-((3-(9-(tetrahydro-2H-pyran-2-yl)-9H-purin-6-yl)pyridin-2-yl)amino)phenyl)isonicotinamide (200 mg, 0.37 mmol, 99%) as an orange solid. MS (ESI) m/z 547.2 [M+H]⁺

Step 4. A solution of 2-cyclopropyl-N-(4-methyl-3-((3-(9-(tetrahydro-2H-pyran-2-yl)-9H-purin-6-yl)pyridin-2-yl)amino)phenyl)isonicotinamide (200 mg, 0.37 mmol) in trifluoroacetic acid/dichloromethane (3 mL/3 mL) was stirred at room temperature for 1 h. The mixture was concentrated. The residue was treated with saturated sodium bicarbonate solution (10 mL), and extracted with N,N-dimethylformamide/ethyl acetate (2 mL/30 mL). The organic layer was concentrated and purified by prep-HPLC (column: Welch Xtimate 21.2*250 mm C18, 10 μm, Mobile Phase: A: water (10 mmol/L ammonium bicarbonate) B: acetonitrile; B %: 30%-70% in 15.0 min) to give N-(3-((3-(9H-purin-6-yl)pyridin-2-yl)amino)-4-methylphenyl)-2-cyclopropyl-isonicotinamide (7.7 mg, 0.02 mmol, 4.5%) as a yellow solid. ¹H NMR (400 MHZ, DMSO-d₆) δ 13.85 (s, 1H), 12.31 (s, 1H), 10.38 (s, 1H), 9.81-9.77 (m, 1H), 9.08 (s, 1H), 8.72 (s, 1H), 8.68 (s, 1H), 8.56 (d, J=5.2 Hz, 1H), 8.36 (dd, J=4.4 Hz, 1.2 Hz, 1H), 7.76 (s, 1H), 7.58 (d, J=4.8 Hz, 1H), 7.46 (d, J=7.2 Hz, 1H), 7.24 (d, J=8.4 Hz, 1H), 7.04 (dd, J=7.6 Hz, 4.4 Hz, 1H), 2.44 (s, 3H), 2.24-2.19 (m, 1H), 1.04-0.96 (m, 4H). MS (ESI) m/z 463.2 [M+H]⁺

### Example 22. Synthesis of N-(3-((3-(9H-purin-6-yl)pyridin-2-ylamino)-4-fluorophenyl)-4-(trifluoromethyl)picolinamide (Compound I-20)

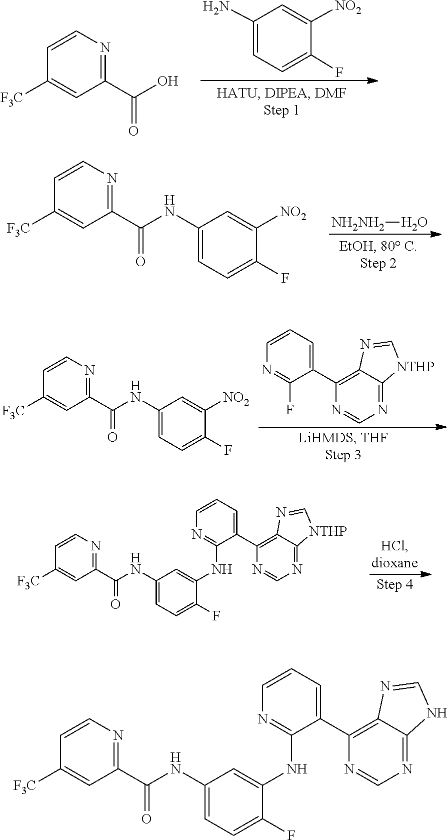

Step 1. A solution of 4-(trifluoromethyl)picolinic acid (167 mg, 0.874 mmol), 4-fluoro-3-nitroaniline (136 mg, 0.874 mmol), 1-[bis(dimethylamino)methylene]-1H-1,2,3-triazolo[4,5-b]pyridinium 3-oxid hexafluorophosphate (498 mg, 1.31 mmol) and N,N-diisopropylethylamine (338 mg, 2.62 mmol) in N,N-dimethylformamide (6 mL) was stirred at 25° C. for 16 h. Then water was added and the mixture was extracted with ethyl acetate. The organic layer was dried and concentrated. The crude residue was purified by silica gel column chromatography (petroleum ether: ethyl acetate from 50:1 to 10:1) to give N-(4-fluoro-3-nitrophenyl)-4-(trifluoromethyl)-picolinamide (200 mg, 0.608 mmol, 70%) as a yellow solid. MS (ESI) m/z 330.1 [M+H]⁺

Step 2. A solution of N-(4-fluoro-3-nitrophenyl)-4-(trifluoromethyl)picolinamide (200 mg, 0.608 mmol), hydrazine hydrate (152 mg, 3.04 mmol) and palladium (10% on carbon, 80 mg) in ethanol (10 mL) was stirred at 80° C. for 1 h. The mixture was concentrated. The crude residue was purified by silica gel column chromatography (dichloromethane: methanol from 50:1 to 10:1) to give N-(3-amino-4-fluorophenyl)-4-(trifluoromethyl)picolinamide (150 mg, 0.502 mmol, 82%) as a yellow solid. MS (ESI) m/z 300.1[M+H]⁺

Step 3. A solution of N-(3-amino-4-fluorophenyl)-4-(trifluoromethyl)picolinamide (150 mg, 0.502 mmol), 6-(2-fluoropyridin-3-yl)-9-(tetrahydro-2H-pyran-2-yl)-9H-purine (165 mg, 0.552 mmol) in tetrahydrofuran (5 mL) was stirred at 0° C. for 10 min. Then lithium hexamethyldisilazide (1.6 M in tetrahydrofuran, 0.94 mL, 1.5 mmol) was added. The mixture was stirred at room temperature for 2 h. The mixture was diluted with ethyl acetate and washed with water. The organic layer was concentrated. The crude residue was purified by silica gel column chromatography (dichloromethane:methanol from 50:1 to 10:1) to give N-(4-fluoro-3-((3-(9-(tetrahydro-2H-pyran-2-yl)-9H-purin-6-yl)pyridin-2-yl)amino)phenyl)-4-(trifluoromethyl)-picolinamide (240 mg, 0.415 mmol, 82%) as a yellow solid. MS (ESI) m/z 579.1 [M+H]⁺

Step 4. A solution of N-(4-fluoro-3-((3-(9-(tetrahydro-2H-pyran-2-yl)-9H-purin-6-yl)pyridin-2-yl) amino)phenyl)-4-(trifluoromethyl)picolinamide (240 mg, 0.415 mmol) and hydrogen chloride solution (4 M in dioxane, 0.52 mL, 2.076 mmol) in 1,4-dioxane (5 mL) was stirred at 25° C. for 1 h. The mixture was concentrated. The crude residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-fluorophenyl)-4-(trifluoromethyl)picolinamide (22.3 mg, 0.045 mmol, 11%) as a yellow solid. ¹H NMR (400 MHz, DMSO-d₆) δ 13.903 (s, 1H), 12.842 (s, 1H), 10.828 (s, 1H), 9.837 (s, 1H), 9.139-9.153 (d, J=5.6 Hz, 1H), 9.054 (s, 2H), 8.738 (s, 1H), 8.434-8.441 (d, J=2.8 Hz, 1H), 8.375 (s, 1H), 8.100-8.110 (d, J=4.0 Hz, 1H), 7.568 (s, 1H), 7.292-7.342 (m, 1H), 7.113-7.143 (m, 1H). MS (ESI) m/z 495.1 [M+H]⁺

### Example 23. Synthesis of N-(3-(3-(9H-purin-6-yl)pyridin-2-ylamino)-4-chlorophenyl)-4-(trifluoromethyl)picolinamide (Compound I-21)

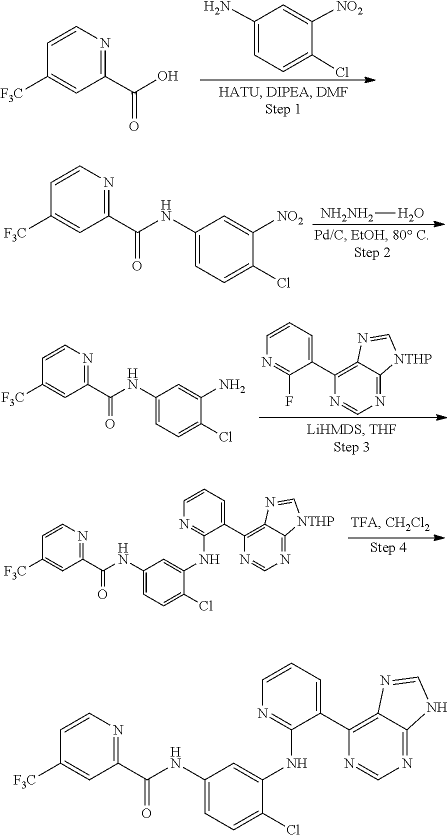

Step 1. A solution of 4-(trifluoromethyl)picolinic acid (260 mg, 1.361 mmol), 4-chloro-3-nitroaniline (234 mg, 1.361 mmol), 1-[bis(dimethylamino)methylene]-1H-1,2,3-triazolo[4,5-b]pyridinium 3-oxid hexafluorophosphate (775 mg, 2.041 mmol) and N,N-diisopropylethylamine (526 mg, 4.082 mmol) in N,N-dimethylformamide (6 mL) was stirred at 25° C. for 16 h. Then water was added and the mixture was extracted with ethyl acetate. The organic layer was dried and concentrated. The crude residue was purified by silica gel column chromatography (petroleum ether: ethyl acetate from 50:1 to 10:1) to give N-(4-chloro-3-nitrophenyl)-4-(trifluoromethyl)-picolinamide (350 mg, 1.014 mmol, 74%) as a yellow solid. MS (ESI) m/z 346.1 [M+H]⁺

Step 2. A solution of N-(4-chloro-3-nitrophenyl)-4-(trifluoromethyl)picolinamide (350 mg, 1.014 mmol), hydrazine hydrate (254 mg, 5.072 mmol) and palladium (10% on carbon, 100 mg) in ethanol (10 mL) was stirred at 80° C. for 1 h. The mixture was concentrated. The crude residue was purified by silica gel column chromatography (dichloromethane: methanol from 50:1 to 10:1) to give N-(3-amino-4-chlorophenyl)-4-(trifluoromethyl)picolinamide (230 mg, 0.73 mmol, 72%) as a yellow solid. MS (ESI) m/z 316.1 [M+H]⁺

Step 3. A solution of N-(3-amino-4-chlorophenyl)-4-(trifluoromethyl)picolinamide (150 mg, 0.476 mmol), 6-(2-fluoropyridin-3-yl)-9-(tetrahydro-2H-pyran-2-yl)-9H-purine (156 mg, 0.524 mmol) in tetrahydrofuran (5 mL) was stirred at 0° C. for 10 min. Then lithium hexamethyldisilazide (1.6 M in tetrahydrofuran, 0.9 mL, 1.4 mmol) was added. The mixture was stirred at room temperature for 2 h. The mixture was diluted with ethyl acetate and washed with water. The organic layer was concentrated. The crude residue was purified by silica gel column chromatography (dichloromethane: methanol from 50:1 to 10:1) to give N-(4-chloro-3-((3-(9-(tetrahydro-2H-pyran-2-yl)-9H-purin-6-yl)pyridin-2-yl)amino)phenyl)-4-(trifluoromethyl)-picolinamide (240 mg, 0.4 mmol, 85%) as a yellow solid. MS (ESI) m/z 594.9 [M+H]⁺

Step 4. A solution of N-(4-chloro-3-((3-(9-(tetrahydro-2H-pyran-2-yl)-9H-purin-6-yl)pyridin-2-yl)amino)phenyl)-4-(trifluoromethyl)picolinamide (300 mg, 0.505 mmol) and hydrogen chloride solution (4 M in dioxane, 0.63 mL, 2.525 mmol) in 1,4-dioxane (5 mL) was stirred at 25° C. for 1 h. The mixture was concentrated. The crude residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-chlorophenyl)-4-(trifluoromethyl) picolinamide (40.6 mg, 0.079 mmol, 16%) as a yellow solid. ¹H NMR (400 MHZ, DMSO-d₆) δ 12.983 (s, 1H), 10.870 (s, 1H), 9.804 (s, 1H), 9.319-9.325 (d, J=2.4 Hz, 1H), 9.105 (s, 2H), 9.049-9.062 (d, J=5.2 Hz, 1H), 8.734 (s, 1H), 8.450-8.466 (m, 1H), 8.373 (s, 1H), 8.101-8.113 (d, J=4.8 Hz, 1H), 7.605-7.633 (m, 1H), 7.512-7.533 (m, 1H), 7.146-7.178 (m, 1H). MS (ESI) m/z 511.0 [M+H]⁺

### Example 24. Synthesis of N-(3-((3-(9H-purin-6-yl)pyridin-2-yl)amino)-4-methylphenyl)-4-chloro-3-cyanobenzamide (Compound I-22)

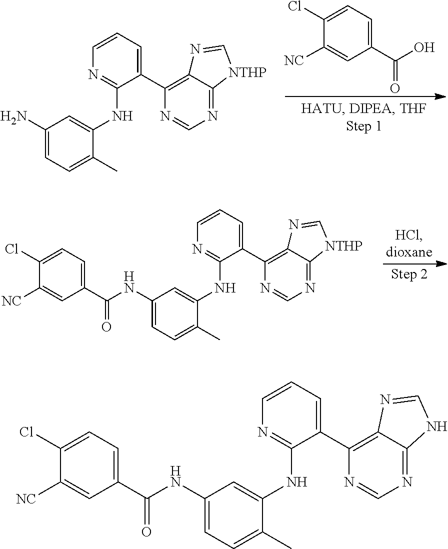

Step 1. A mixture of 4-chloro-3-cyanobenzoic acid (40 mg, 0.22 mmol), diisopropylethylamine (57 mg, 0.44 mmol) and 1-[bis(dimethylamino)methylene]-1H-1,2,3-triazolo[4,5-b]pyridinium 3-oxid hexafluorophosphate (125 mg, 0.33 mmol) in tetrahydrofuran (8 mL) was stirred at room temperature for 30 min, then 6-methyl-N1-(3-(9-(tetrahydro-2H-pyran-2-yl)-9H-purin-6-yl)pyridin-2-yl)benzene-1,3-diamine (90 mg, 0.22 mmol) was added. The mixture was stirred at room temperature for 1 h. The mixture was concentrated and purified by flash chromatography (methanol/dichloromethane=4/96) to give 4-chloro-3-cyano-N-(4-methyl-3-((3-(9-(tetrahydro-2H-pyran-2-yl)-9H-purin-6-yl) pyridin-2-yl)amino)phenyl)benzamide (110 mg, 0.195 mmol, 88%) as a yellow solid. MS (ESI) m/z 565.2 [M+H]⁺

Step 2. To a solution of 4-chloro-3-cyano-N-(4-methyl-3-((3-(9-(tetrahydro-2H-pyran-2-yl)-9H-purin-6-yl)pyridin-2-yl)amino)phenyl)benzamide (110 mg, 0.195 mmol) in dioxane (2 mL) was added hydrogen chloride solution (4 M in dioxane, 2 mL). The mixture was stirred at 25° C. for 3 h. After completed, the reaction mixture was concentrated and the pH was adjusted to 8 by addition of 7 N ammonia solution in methanol. The mixture concentrated under reduced pressure, treated with methanol (10 mL), and filtered. The solid was suspended in water (10 mL) and freeze-dried under vacuum to afford N-(3-((3-(9H-purin-6-yl)pyridin-2-yl)amino)-4-methylphenyl)-4-chloro-3-cyanobenzamide (89.2 mg, 0.186 mmol, 95%) as a yellow solid. ¹H NMR (400 MHZ, DMSO-d₆) δ 13.86 (d, J=3.2 Hz, 1H), 12.36 (s, 1H), 10.43 (s, 1H), 9.85-9.83 (m, 1H), 9.09 (s, 1H), 8.69 (s, 1H), 8.58 (s, 2H), 8.38-8.36 (m, 1H), 8.29-8.26 (m, 1H), 7.93 (d, J=8.4 Hz, 1H), 7.48 (d, J=8.4 Hz, 1H), 7.25 (d, J=8.4 Hz, 1H), 7.07-7.04 (m, 1H), 2.45 (s, 3H). MS (ESI) m/z 480.9 [M+H]⁺

### Example 25. Synthesis of N-(3-((3-(9H-purin-6-yl)pyridin-2-yl)amino)-4-methylphenyl)-4-fluorobenzamide (Compound I-23)

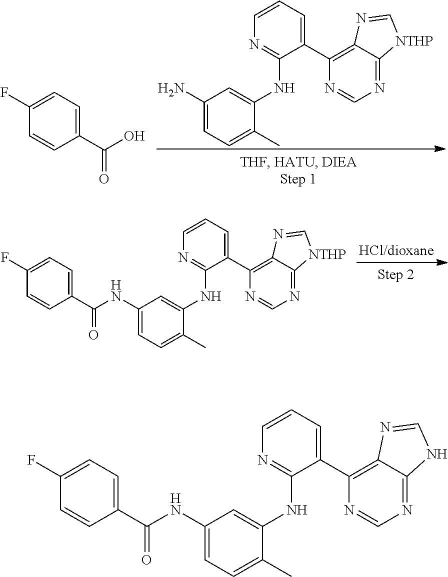

Step 1. To a mixture of 4-fluorobenzoic acid (50 mg, 0.36 mmol) in tetrahydrofuran (3 mL) was added N,N-diisopropylethylamine (1.07 mmol, 0.2 mL) and 1-[bis(dimethylamino)methylene]-1H-1,2,3-triazolo[4,5-b]pyridinium 3-oxid hexafluorophosphate (204 mg 0.54 mmol). The mixture was stirred at room temperature for 0.5 h. Then 6-methyl-N1-(3-(9-(tetrahydro-2H-pyran-2-yl)-9H-purin-6-yl)pyridin-2-yl) benzene-1,3-diamine (143 mg, 0.36 mmol) was added, and the mixture was stirred at room temperature for 1 h. The reaction mixture was concentrated. The residue was purified by flash chromatography (petroleum ether/ethyl acetate=2/1) to give 4-fluoro-N-(4-methyl-3-((3-(9-(tetrahydro-2H-pyran-2-yl)-9H-purin-6-yl)pyridin-2-yl)amino)-phenyl)benzamide (70 mg, 0.13 mmol, 38%) as a white solid. MS (ESI) m/z 523.9 [M+H]⁺

Step 2. To a mixture of 4-fluoro-N-(4-methyl-3-((3-(9-(tetrahydro-2H-pyran-2-yl)-9H-purin-6-yl) pyridin-2-yl)amino)phenyl)benzamide (50 mg, 0.09 mmol) in dioxane (2 mL) was added hydrogen chloride solution (4 M in dioxane, 5 mL) at room temperature. Then the reaction mixture was stirred at room temperature for 2 h. The reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl) amino)-4-methylphenyl)-4-fluorobenzamide (20.9 mg, 0.05 mmol, 51%) as a yellow solid. ¹H NMR (400 MHZ, DMSO-d₆) δ 2.43 (s, 3H), 7.01-7.04 (m, 1H), 7.22 (d, J=8.5 Hz, 1H), 7.35 (t, J=9.0 Hz, 2H), 7.45 (d, J=7.0 Hz, 1H), 8.04-8.06 (m, 2H), 8.34 (d, J=3.0 Hz, 1H), 8.64 (s, 1H), 8.69 (s, 1H), 9.07 (s, 1H), 9.78 (d, J=7.0 Hz, 1H), 10.21 (s, 1H), 12.29 (s, 1H). MS (ESI) m/z 440.3 [M+H]⁺

### Example 26. Synthesis of N-(3-((3-(9H-purin-6-yl)pyridin-2-yl)amino)-4-methylphenyl)-4-(trifluoromethyl)benzamide (Compound I-24)

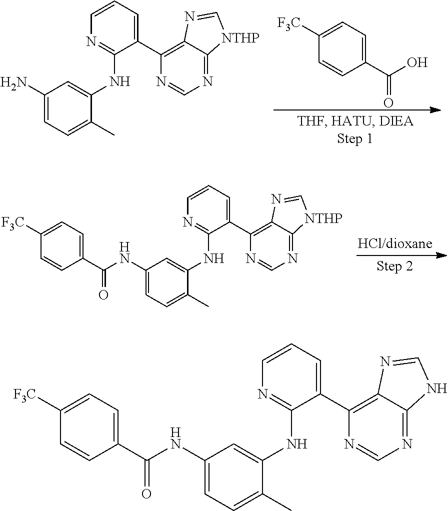

Step 1. To a mixture of 4-(trifluoromethyl)benzoic acid (47 mg, 0.249 mmol) in tetrahydrofuran (3 mL) was added N,N-diisopropylethylamine (0.748 mmol, 0.12 mL) and 1-[bis(dimethylamino)methylene]-1H-1,2,3-triazolo[4,5-b]pyridinium 3-oxid hexafluorophosphate (142 mg, 0.374 mmol). The mixture was stirred at room temperature for 0.5 h. Then 6-methyl-N1-(3-(9-(tetrahydro-2H-pyran-2-yl)-9H-purin-6-yl) pyridin-2-yl)benzene-1,3-diamine (100 mg, 0.249 mmol) was added, and the mixture was stirred at room temperature for 1 h. The reaction mixture was concentrated. The residue was purified by flash chromatography (petroleum ether/ethyl acetate=2/1) to give N-(4-methyl-3-((3-(9-(tetrahydro-2H-pyran-2-yl)-9H-purin-6-yl)pyridin-2-yl)amino)phenyl)-4-(trifluoromethyl)benzamide (75 mg, 0.13 mmol, 54%) as a white solid. MS (ESI) m/z 573.9 [M+H]⁺

Step 2. To a mixture of N-(4-methyl-3-((3-(9-(tetrahydro-2H-pyran-2-yl)-9H-purin-6-yl)pyridin-2-yl)amino)phenyl)-4-(trifluoromethyl)benzamide (60 mg, 0.10 mmol) in dioxane (2 mL) was added hydrogen chloride solution (4 M in dioxane, 5 mL) at room temperature. Then the reaction mixture was stirred at room temperature for 2 h. The reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl) pyridin-2-yl)amino)-4-methylphenyl)-4-(trifluoromethyl)-benzamide (12.3 mg, 0.025 mmol, 24%) as a yellow solid. ¹H NMR (500 MHZ, DMSO-d₆) δ 13.85 (s, 1H), 12.30 (s, 1H), 10.43 (s, 1H), 9.80 (s, 1H), 9.08 (s, 1H), 8.72 (s, 1H), 8.68 (d, J=2.0 Hz, 1H) 8.34-8.36 (m, 1H), 8.16 (d, J=8.5 Hz, 2H), 7.90 (d, J=8.0 Hz, 2H), 7.48 (dd, J1=1.5 Hz, J2=8.25 Hz, 1H), 7.24 (d, J=8.5 Hz, 1H) 7.02-7.05 (m, 1H), 2.44 (s, 3H). MS (ESI) m/z 490.2 [M+H]⁺

### Example 27. Synthesis of N-(3-(3-(9H-purin-6-yl)pyridin-2-ylamino)-4-methylphenyl)-5-chloro-4-(trifluoromethyl)picolinamide (Compound I-25)

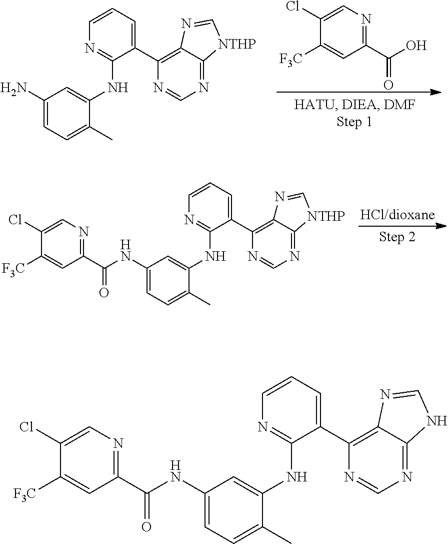

Step 1. A solution of 5-chloro-4-(trifluoromethyl)picolinic acid (38 mg, 0.169 mmol), 6-methyl-N1-(3-(9-(tetrahydro-2H-pyran-2-yl)-9H-purin-6-yl)pyridin-2-yl)benzene-1,3-diamine (68 mg, 0.169 mmol), 1-[bis(dimethylamino)methylene]-1H-1,2,3-triazolo[4,5-b]pyridinium 3-oxid hexafluorophosphate (96 mg, 0.253 mmol) and N,N-diisopropylethylamine (65 mg, 0.507 mmol) in N,N-dimethylfomamide (4 mL) was stirred at 25° C. for 1 h. Then water was added and the mixture was extracted with ethyl acetate. The organic layer was dried and concentrated. The crude residue was purified by silica gel column chromatography (petroleum ether: ethyl acetate from 50:1 to 10:1) to give 5-chloro-N-(4-methyl-3-((3-(9-(tetrahydro-2H-pyran-2-yl)-9H-purin-6-yl)pyridin-2-yl)amino)phenyl)-4-(trifluoromethyl)picolinamide (75 mg, 0.123 mmol, 73%) as a yellow solid. MS (ESI) m/z 609.1 [M+H]⁺

Step 2. A solution of 5-chloro-N-(4-methyl-3-((3-(9-(tetrahydro-2H-pyran-2-yl)-9H-purin-6-yl)pyridin-2-yl)amino)phenyl)-4-(trifluoromethyl)picolinamide (75 mg, 0.123 mmol) in hydrogen chloride solution (4 M in dioxane, 5 mL) was stirred at 25° C. for 1 h. The mixture was concentrated. The crude residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-5-chloro-4-(trifluoromethyl)picolinamide (16.1 mg, 0.03 mmol, 25%) as a yellow solid. ¹H NMR (400 MHZ, DMSO-d₆) δ 12.522 (s, 1H), 10.657 (s, 1H), 9.857 (s, 1H), 9.101 (s, 1H), 8.984 (s, 1H), 8.847 (s, 1H), 8.571 (s, 1H), 8.396 (s, 1H), 8.333 (s, 1H), 7.503-7.522 (d, J=7.6 Hz, 1H), 7.239-7.257 (d, J=7.2 Hz, 1H), 7.028 (s, 1H), 2.454 (s, 3H). MS (ESI) m/z 525.1 [M+H]⁺

### Example 28. Synthesis of N-(3-((3-(9H-purin-6-yl)pyridin-2-yl)amino)-4-methylphenyl)-4-cyanobenzamide (Compound I-26)

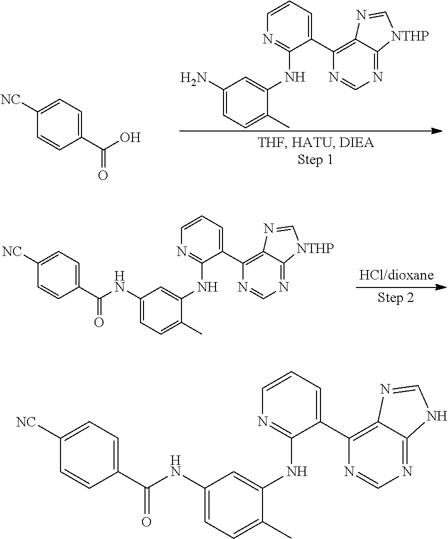

Step 1. To a mixture of 4-cyanobenzoic acid (25 mg, 0.17 mmol) in tetrahydrofuran (3 mL) was added N,N-diisopropylethylamine (39 mg, 0.31 mmol) and 1-[bis(dimethylamino)methylene]-1H-1,2,3-triazolo[4,5-b]pyridinium 3-oxid hexafluorophosphate (71 mg, 0.18 mmol). The mixture was stirred at 25° C. for 0.5 h. Then 6-methyl-N1-(3-(9-(tetrahydro-2H-pyran-2-yl)-9H-purin-6-yl)pyridin-2-yl)benzene-1,3-diamine (62 mg, 0.15 mmol) was added, and the mixture was stirred at 25° C. for 1 h. The reaction mixture was concentrated. The residue was purified by flash chromatography (petroleum ether/ethyl acetate=2/1) to give 4-cyano-N-(4-methyl-3-((3-(9-(tetrahydro-2H-pyran-2-yl)-9H-purin-6-yl)pyridin-2-yl)amino) phenyl)benzamide (50 mg, 0.09 mmol, 61%) as a yellow solid. MS (ESI) m/z 531.0 [M+H]⁺

Step 2. To a mixture of 4-cyano-N-(4-methyl-3-((3-(9-(tetrahydro-2H-pyran-2-yl)-9H-purin-6-yl) pyridin-2-yl)amino)phenyl)benzamide (50 mg, 0.09 mmol) in dioxane (2 mL) was added hydrogen chloride solution (4 M in dioxane, 5 mL) at 25° C. Then the reaction mixture was stirred at 25° C. for 2 h. The reaction mixture was concentrated. The residual was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-4-cyanobenzamide (11.5 mg, 0.025 mmol, 28%) as a yellow solid. ¹H NMR (400 MHZ, DMSO-d₆) δ 12.33 (s, 1H), 10.46 (s, 1H), 9.84 (s, 1H), 9.10 (s, 1H), 8.71 (d, J=15.2 Hz, 1H), 8.35 (d, J=2.8 Hz, 1H), 8.12 (d, J=8.4 Hz, 1H), 8.02 (d, J=8.4 Hz, 1H), 7.48 (d, J=7.2 Hz, 1H), 7.24 (d, J=8.8 Hz, 1H), 7.04 (dd, J=8.0, 4.4 Hz, 1H), 2.45 (s, 1H). MS (ESI) m/z 447.0 [M+H]⁺

### Example 29. Synthesis of N-(3-((3-(9H-purin-6-yl)pyridin-2-yl)amino)-4-methylphenyl)-4-methylbenzamide (Compound I-27)

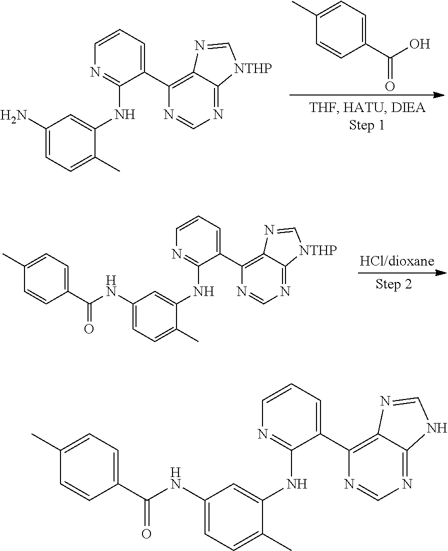

Step 1. To a mixture of 4-methylbenzoic acid (34 mg, 0.25 mmol) in tetrahydrofuran (3 mL) was added N,N-diisopropylethylamine (97 mg, 0.75 mmol, 0.12 mL) and 1-[bis(dimethylamino)methylene]-1H-1,2,3-triazolo[4,5-b]pyridinium 3-oxid hexafluorophosphate (142 mg, 0.37 mmol). The mixture was stirred at room temperature for 0.5 h. Then 6-methyl-N1-(3-(9-(tetrahydro-2H-pyran-2-yl)-9H-purin-6-yl)pyridin-2-yl)benzene-1,3-diamine (100 mg, 0.25 mmol) was added, and the mixture was stirred at room temperature for 1 h. The reaction mixture was concentrated. The residue was purified by flash chromatography (petroleum ether/ethyl acetate=1/1) to give 4-methyl-N-(4-methyl-3-((3-(9-(tetrahydro-2H-pyran-2-yl)-9H-purin-6-yl)pyridin-2-yl)amino)phenyl)benzamide (120 mg, 0.23 mmol, 93%) as a white solid. MS (ESI) m/z 519.9 [M+H]⁺

Step 2. To a mixture of 4-methyl-N-(4-methyl-3-((3-(9-(tetrahydro-2H-pyran-2-yl)-9H-purin-6-yl) pyridin-2-yl)amino)phenyl)benzamide (110 mg, 0.21 mmol) in dioxane (2 mL) was added hydrogen chloride solution (4 M in dioxane, 5 mL) at room temperature. Then the reaction mixture was stirred at room temperature for 2 h. The reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-4-methylbenzamide (13.7 mg, 0.03 mmol, 15%) as a yellow solid. ¹H NMR (500 MHZ, DMSO-d₆) δ 2.38 (s, 3H), 2.42 (s, 3H), 7.02 (q, J=5.0 Hz, 1H), 7.21 (d, J=8.5 Hz, 1H), 7.32 (d, J=8.0 Hz, 2H), 7.46 (d, J=7.5 Hz, 1H), 7.89 (d, J=8.0 Hz, 2H), 8.34 (q, J=2.0 Hz, 1H), 8.62 (s, 1H), 8.71 (s, 1H), 9.08 (s, 1H), 9.81 (d, J=7.5 Hz, 1H), 10.10 (s, 1H), 12.28 (s, 1H), 13.85 (s, 1H). MS (ESI) m/z 435.9 [M+H]⁺

### Example 30. Synthesis of N-(3-(3-(9H-purin-6-yl)pyridin-2-ylamino)-4-methylphenyl)-3-chlorobicyclo[1.1.1]pentane-1-carboxamide (Compound I-28)

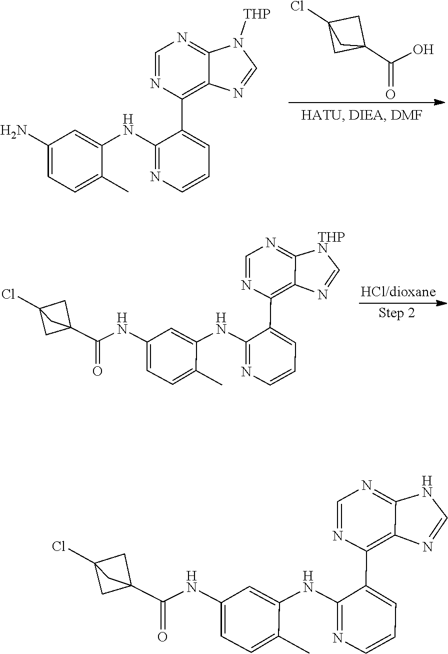

Step 1. A solution of 6-methyl-N1-(3-(9-(tetrahydro-2H-pyran-2-yl)-9H-purin-6-yl)pyridin-2-yl)benzene-1,3-diamine (42 mg, 0.22 mmol), 3-chlorobicyclo[1.1.1]pentane-1-carboxylic acid (64 mg, 0.18 mmol), 1-[bis(dimethylamino)methylene]-1H-1,2,3-triazolo[4,5-b]pyridinium 3-oxid hexafluorophosphate (114 mg, 0.3 mmol) and N,N-diisopropylethylamine (81 mg, 0.63 mmol) in N,N-dimethylformamide (3 mL) was stirred at 25° C. for 1 h. The reaction mixture was diluted with ethyl acetate (50 mL), then washed with water (25 mL) and brine (25 mL×3), dried over sodium sulfate, filtered and concentrated under reduced pressure. The residue was purified by column chromatography (ethyl acetate/petroleum ether=75/100) to give 3-chloro-N-(4-methyl-3-(3-(9-(tetrahydro-2H-pyran-2-yl)-9H-purin-6-yl)pyridin-2-ylamino)phenyl)-bicyclo[1.1.1]pentane-1-carboxamide (115 mg, 0.22 mmol, 88%) as a yellow solid. MS (ESI) m/z 530.0 [M+H]⁺

Step 2. To a solution of 3-chloro-N-(4-methyl-3-(3-(9-(tetrahydro-2H-pyran-2-yl)-9H-purin-6-yl)pyridin-2-ylamino)phenyl)bicyclo[1.1.1]pentane-1-carboxamide (115 mg, 0.22 mmol) in dioxane (10 mL) was added hydrogen chloride solution (4 M in dioxane, 2 mL) at 25° C., and stirred at 25° C. for 1 h. After completed, the reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-(3-(9H-purin-6-yl)pyridin-2-ylamino)-4-methylphenyl)-3-chlorobicyclo[1.1.1]pentane-1-carboxamide (45.1 mg, 0.1 mmol, 45%) as a yellow solid. ¹H NMR (400 MHz, DMSO-d₆) δ 12.27 (s, 1H), 9.77 (d, J=7.2 Hz, 1H), 9.65 (s, 1H), 9.06 (s, 1H), 8.71 (s, 1H), 8.49 (d, J=1.6 Hz, 1H), 8.36 (dd, J=4.6, 1.7 Hz, 1H), 7.33 (dd, J=8.2, 1.7 Hz, 1H), 7.17 (d, J=8.3 Hz, 1H), 7.03 (dd, J=7.8, 4.7 Hz, 1H), 2.49 (s, 6H), 2.41 (s, 3H). MS (ESI) m/z 446.1 [M+H]⁺

### Example 31. Synthesis of N-(3-(3-(9H-purin-6-yl)pyridin-2-ylamino)-5-fluoro-4-methylphenyl)-4-(trifluoromethyl)picolinamide (Compound I-29)

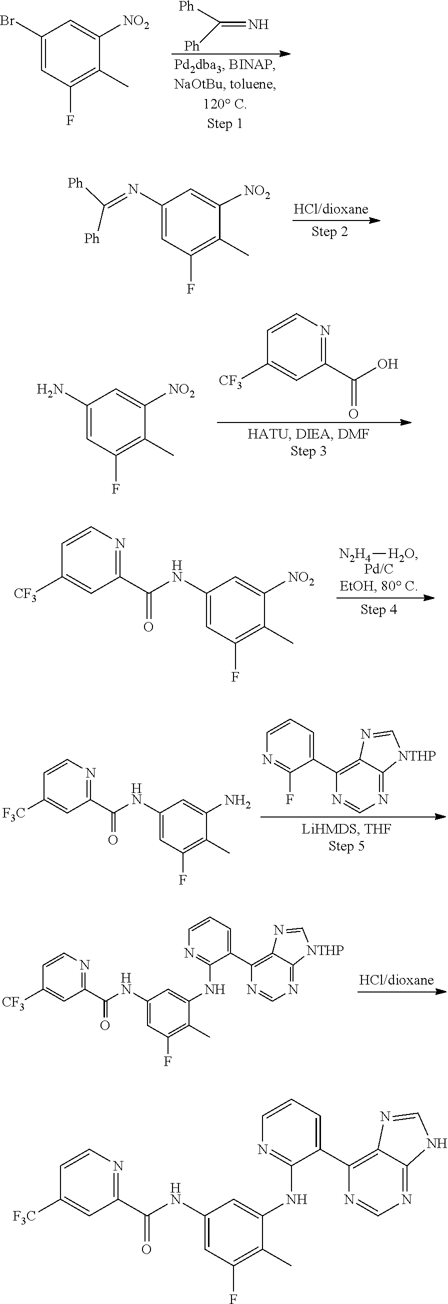

Step 1. A solution of 5-bromo-1-fluoro-2-methyl-3-nitrobenzene (520 mg, 2.231 mmol), diphenylmethanimine (609 mg, 3.347 mmol), tri(dibenzylideneacetone)dipalladium (204 mg, 0.223 mmol), (+)-2,2′-bis (diphenylphosphino)-1,1′-binaphthalene (235 mg, 0.446 mmol) and sodium tert-butoxide (642 mg, 6.693 mmol) in toluene (10 mL) was stirred at 120° C. for 16 h. On completion, water was added and the mixture was extracted with ethyl acetate (30 mL×3). The organic layer was dried and concentrated. The residue was purified by column chromatography (petroleum ether/ethyl acetate=20/1 to 10/1) to give N-(3-fluoro-4-methyl-5-nitrophenyl)-1,1-diphenylmethanimine (400 mg, 1.198 mmol, 54%) as a white solid. MS (ESI) m/z 335.1 [M+H]⁺

Step 2. A solution of N-(3-fluoro-4-methyl-5-nitrophenyl)-1,1-diphenylmethanimine (400 mg, 1.198 mmol) and hydrogen chloride solution (4 M in dioxane, 1.5 mL, 5.988 mmol) in 1,4-dioxane (5 mL) was stirred at 25° C. for 1 h. The mixture was concentrated. The crude residue was purified by silica gel column chromatography (petroleum ether/ethyl acetate=20/1 to 5/1) to give 3-fluoro-4-methyl-5-nitroaniline (130 mg, 0.76 mmol, 64%) as a yellow solid. MS (ESI) m/z 171.1[M+H]⁺

Step 3. A solution of 4-(trifluoromethyl)picolinic acid (146 mg, 0.765 mmol), 3-fluoro-4-methyl-5-nitroaniline (130 mg, 0.765 mmol), 1-[bis(dimethylamino)methylene]-1H-1,2,3-triazolo[4,5-b]pyridinium 3-oxid hexafluorophosphate (1.147 mg, 0.436 mmol) and N,N-diisopropylethylamine (296 mg, 2.295 mmol) in N,N-dimethyl formamide (5 mL) was stirred at 25° C. for 1 h. Then water was added and the mixture was extracted with ethyl acetate (30 mL×3). The organic layer was dried and concentrated. The crude residue was purified by silica gel column chromatography (petroleum ether/ethyl acetate=20/1 to 5/1) to give N-(3-fluoro-4-methyl-5-nitrophenyl)-4-(trifluoromethyl)picolinamide (200 mg, 0.58 mmol, 76%) as a yellow solid. MS (ESI) m/z 344.1 [M+H]⁺

Step 4. A solution of N-(3-fluoro-4-methyl-5-nitrophenyl)-4-(trifluoromethyl)-picolinamide (200 mg, 0.58 mmol), hydrazine hydrate (145 mg, 2.915 mmol) and palladium (10% on carbon, 80 mg) in EtOH (10 mL) was stirred at 80° C. for 1 h. The mixture was concentrated. The crude residue was purified by silica gel column chromatography (dichloromethane/methanol=50/1 to 10/1) to give N-(3-amino-5-fluoro-4-methylphenyl)-4-(trifluoromethyl)picolinamide (130 mg, 0.415 mmol, 71%) as a yellow solid. MS (ESI) m/z 314.1[M+H]⁺

Step 5. A solution of N-(3-fluoro-4-methyl-5-nitrophenyl)-4-(trifluoromethyl)-picolinamide (90 mg, 0.287 mmol), 6-(2-fluoropyridin-3-yl)-9-(tetrahydro-2H-pyran-2-yl)-9H-purine (95 mg, 0.316 mmol) in tetrahydrofuran (5 mL) was stirred at 0° C. for 10 min. Then lithium hexamethyldisilazide (0.27 mL, 0.431 mmol) was added. The mixture was stirred at room temperature for 2 h. The mixture was dilute with water (30 mL) and extracted with ethyl acetate (30 mL×3). The organic layer was concentrated. The crude residue was purified by silica gel column chromatography (dichloromethane/methanol=50/1 to 10/1) to give N-(3-fluoro-4-methyl-5-((3-(9-(tetrahydro-2H-pyran-2-yl)-9H-purin-6-yl)pyridin-2-yl)amino)phenyl)-4-(trifluoromethyl)picolinamide (100 mg, 0.169 mmol, 59%) as a yellow solid. MS (ESI) m/z 593.1 [M+H]⁺

Step 6. A solution of N-(3-fluoro-4-methyl-5-((3-(9-(tetrahydro-2H-pyran-2-yl)-9H-purin-6-yl)pyridin-2-yl)amino)phenyl)-4-(trifluoromethyl)picolinamide (100 mg, 0.169 mmol) in hydrogen chloride solution (4 M in dioxane, 5 mL) was stirred at 25° C. for 1 h. The mixture was concentrated. The crude residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-5-fluoro-4-methylphenyl)-4-(trifluoromethyl)picolinamide (24.0 mg, 0.047 mmol, 28%) as a yellow solid. ¹H NMR (400 MHZ, DMSO-d₆) δ 13.867-13.885 (d, J=7.2 Hz, 1H), 12.455 (s, 1H), 10.850 (s, 1H), 9.815 (s, 1H), 9.099 (s, 1H), 9.041-9.053 (d, J=4.8 Hz, 1H), 8.733 (s, 2H), 8.395-8.403 (d, J=3.2 Hz, 1H), 8.361 (s, 1H), 8.097-8.109 (d, J=4.8 Hz, 1H), 7.577-7.607 (d, J=12.0 Hz, 1H), 7.079-7.110 (m, 1H), 2.348 (s, 3H). MS (ESI) m/z 509.1 [M+H]⁺

### Example 32. Synthesis of 3-((3-(9H-Purin-6-yl)pyridin-2-yl)amino)-4-methyl-N-(3-(trifluoromethyl)phenyl)benzamide (Compound II-3)

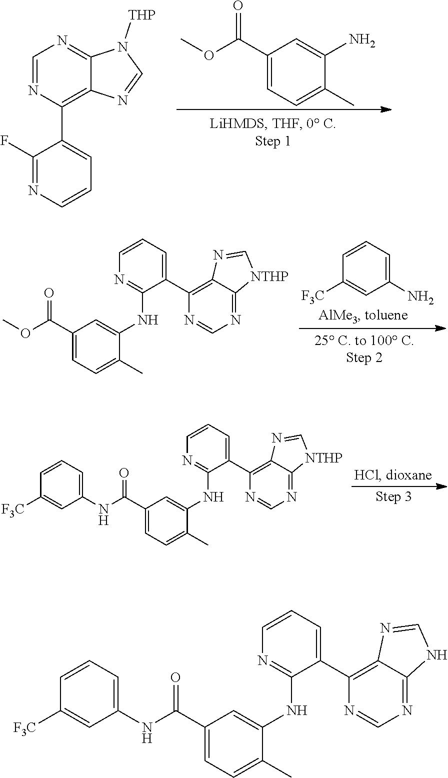

Step 1. To a solution of methyl 3-amino-4-methylbenzoate (276 mg, 1.67 mmol), 6-(2-fluoropyridin-3-yl)-9-(tetrahydro-2H-pyran-2-yl)-9H-purine (500 mg, 1.67 mmol) in tetrahydrofuran (20 mL) was added lithium hexamethyldisilazide (3.1 mL, 5.01 mmol, 1.6 M in tetrahydrofuran) at 0° C. slowly. The mixture was stirred at 0° C. for 1 h. The reaction was quenched with saturated ammonium chloride solution (3 mL) at 0° C. and extracted with dichloromethane (60 mL×3). The combined organic layer was washed with brine (50 mL), dried over sodium sulfate and filtered. The filtrate was concentrated under reduced pressure. The crude residue was purified by flash chromatography (ethyl acetate/petroleum ether=55/100) to give methyl 4-methyl-3-((3-(9-(tetrahydro-2H-pyran-2-yl)-9H-purin-6-yl)pyridin-2-yl)amino)benzoate (200 mg, 0.45 mmol, 27%) as a yellow solid. MS (ESI) m/z 445.2 [M+H]⁺

Step 2. To a solution of 3-(trifluoromethyl)aniline (63 mg, 0.39 mmol) in toluene (5 mL) was added trimethylaluminium (0.9 mL, 0.3 mmol) slowly at 25° C. under argon. The mixture was stirred at 25° C. for 2 h. Then methyl 4-methyl-3-((3-(9-(tetrahydro-2H-pyran-2-yl)-9H-purin-6-yl)pyridin-2-yl)amino)benzoate (132 mg, 0.1 mmol) in toluene (5.0 mL) was added to the mixture. The resulting mixture was heated to 100° C. and stirred for 3 h. After completed, the mixture was cooled to 25° C., ice was added slowly, then water and ethyl acetate was added. The mixture was filtered and washed with ethyl acetate. The filtrate was washed with water (50 mL×3). The organic layer was dried over sodium sulfate, filtered, and concentrated. The residue was purified by flash chromatography (ethyl acetate in petroleum ether from 5% to 48%) to give 4-methyl-3-((3-(9-(tetrahydro-2H-pyran-2-yl)-9H-purin-6-yl)pyridin-2-yl)amino)-N-(3-(trifluoromethyl)-phenyl)benzamide (120 mg, 0.21 mmol, 70%) as a yellow solid. MS (ESI) m/z 574.2 [M+H]⁺

Step 3. To a solution of 4-methyl-3-((3-(9-(tetrahydro-2H-pyran-2-yl)-9H-purin-6-yl)pyridin-2-yl)amino)-N-(3-(trifluoromethyl)phenyl)benzamide (120 mg, 0.21 mmol) in dioxane (10 mL) was added hydrogen chloride solution (4 M in dioxane, 2 mL) at 25° C., and the mixture was stirred at 25° C. for 1 h. After completed, the reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 3-((3-(9H-purin-6-yl)pyridin-2-yl)amino)-4-methyl-N-(3-(trifluoromethyl)phenyl)benzamide (44.6 mg, 0.09 mmol, 43%) as a yellow solid. ¹H NMR (400 MHZ, DMSO-d₆) δ 13.86 (s, 1H), 12.44 (s, 1H), 10.48 (s, 1H), 9.82 (s, 1H), 9.11 (s, 1H), 8.95 (d, J=1.5 Hz, 1H), 8.73 (s, 1H), 8.38 (dd, J=4.7, 1.9 Hz, 1H), 8.28 (s, 1H), 8.07 (d, J=8.1 Hz, 1H), 7.67-7.54 (m, 2H), 7.49-7.39 (m, 2H), 7.07 (dd, J=7.9, 4.7 Hz, 1H), 2.51 (s, 3H). MS (ESI) m/z 489.9 [M+H]⁺

### Example 33. Synthesis of 3-((3-(9H-purin-6-yl)pyridin-2-yl)amino)-4-methyl-N-(5-(trifluoromethyl)pyridin-3-yl)benzamide (Compound I-30)

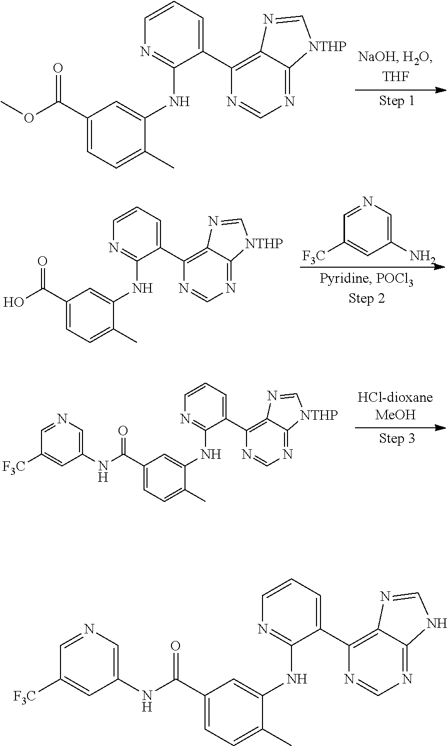

Step 1. To a solution of methyl 4-methyl-3-((3-(9-(tetrahydro-2H-pyran-2-yl)-9H-purin-6-yl)pyridin-2-yl)amino)benzoate (200 mg, 0.45 mmol) in tetrahydrofuran (6 mL) were added sodium hydroxide (180 mg, 4.5 mmol) and water (6 mL), and the mixture was stirred at 25° C. for 96 h. The solvent was evaporated under reduced pressure, and the residue was diluted with water (5 mL). The mixture was adjusted to pH 3 by adding 1 N hydrochloric acid slowly. The resulting white precipitate was collected by filtration and washed with water (5 mL) to give 4-methyl-3-((3-(9-(tetrahydro-2H-pyran-2-yl)-9H-purin-6-yl)pyridin-2-yl)amino)benzoic acid (100 mg, 0.23 mmol, 51%) as a yellow solid. MS (ESI) m/z 431.1 [M+H]⁺

Step 2. To a mixture of 4-methyl-3-((3-(9-(tetrahydro-2H-pyran-2-yl)-9H-purin-6-yl)pyridin-2-yl)amino)benzoic acid (110 mg, 0.26 mmol) and 5-(trifluoromethyl)pyridin-3-amine (84 mg, 0.525 mmol) in pyridine (2.0 mL) was added phosphorus oxychloride (198 mg, 1.3 mmol), and the mixture was stirred at 0° C. for 5 minutes. The mixture was diluted with ethyl acetate (30 mL) and washed with saturated sodium bicarbonate (10 mL) and brine (25 mL). The organic layer was dried with sodium sulphate, filtered, and concentrated. The residue was purified by flash chromatography (100% ethyl acetate) to give 4-methyl-3-((3-(9-(tetrahydro-2H-pyran-2-yl)-9H-purin-6-yl)pyridin-2-yl)amino)-N-(5-(trifluoromethyl)pyridin-3-yl)benzamide (50 mg, 0.087 mmol, 34%) as a yellow solid. MS (ESI) m/z 574.8 [M+H]⁺

Step 3. A solution of 4-methyl-3-((3-(9-(tetrahydro-2H-pyran-2-yl)-9H-purin-6-yl)pyridin-2-yl)amino)-N-(5-(trifluoromethyl)pyridin-3-yl)benzamide (50 mg, 0.087 mmol) in hydrogen chloride solution (4 M in dioxane, 2 mL) and methanol (1 mL) was stirred at room temperature for 2 h. The mixture was concentrated in vacuo. The residue was triturated with methanol (3 mL) to give 3-((3-(9H-purin-6-yl)pyridin-2-yl)amino)-4-methyl-N-(5-(trifluoromethyl)pyridin-3-yl)benzamide (29.5 mg, 0.06 mmol, 70%) as a yellow solid. ¹H NMR (400 MHZ, DMSO-d₆) δ 13.89 (bs, 1H), 12.48 (s, 1H), 10.72 (s, 1H), 9.83 (bm, 1H), 9.22 (d, J=1.2 Hz, 1H), 9.10 (d, J=2.4 Hz, 1H), 9.01 (d, J=1.2 Hz, 1H), 8.73-8.67 (m, 3H), 8.39-8.38 (m, 1H), 7.64 (dd, J=8.0 Hz, 1.2 Hz, 1H), 7.47 (d, J=8.0 Hz, 1H), 7.08 (dd, J=7.6 Hz, 4.4 Hz, 1H), 2.56 (s, 3H). MS (ESI) m/z 491.1 [M+H]⁺

### Example 34. Synthesis of N-(3-((3-(9H-purin-6-yl)pyridin-2-yl)amino)-4-methylphenyl)-4-ethynylbenzamide (Compound I-31)

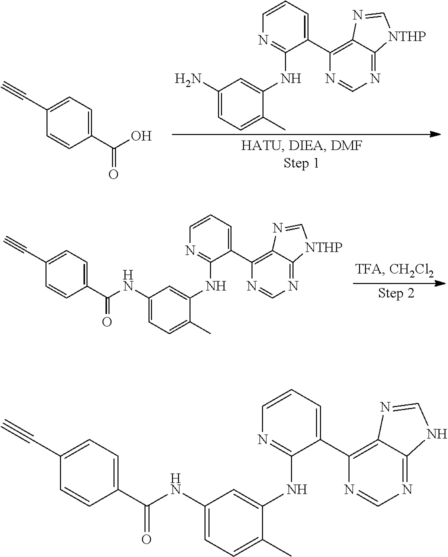

Step 1. A solution of 4-ethynylbenzoic acid (50 mg, 0.34 mmol), 6-methyl-N1-(3-(9-(tetrahydro-2H-pyran-2-yl)-9H-purin-6-yl)pyridin-2-yl)benzene-1,3-diamine (136 mg, 0.34 mmol), 1-[bis(dimethylamino)methylene]-1H-1,2,3-triazolo[4,5-b]pyridinium 3-oxid hexafluorophosphate (142 mg, 0.37 mmol) and N,N-diisopropylethylamine (88 mg, 0.68 mmol) in N,N-dimethylformamide (2.0 mL) was stirred at room temperature for 16 h. The reaction mixture was diluted with ethyl acetate (80 mL) and washed with water (20 mL×3), and brine (30 mL). The organic layer was dried over sodium sulfate, filtered, and concentrated. The residue was purified by flash chromatography (petroleum ether/ethyl acetate=1/1) to give 4-ethynyl-N-(4-methyl-3-((3-(9-(tetrahydro-2H-pyran-2-yl)-9H-purin-6-yl)pyridin-2-yl)amino)phenyl)-benzamide (80 mg, 0.15 mmol, 45%) as a yellow solid. MS (ESI) m/z 529.8 [M+H]⁺

Step 2. A solution of 4-ethynyl-N-(4-methyl-3-((3-(9-(tetrahydro-2H-pyran-2-yl)-9H-purin-6-yl)pyridine-2-yl)amino)phenyl)benzamide (80 mg, 0.15 mmol) in trifluoroacetic acid/dichloromethane (3 mL/3 mL) was stirred at room temperature for 1 h. The mixture was concentrated, the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-4-ethynylbenzamide (38.9 mg, 0.09 mmol, 58%) as a yellow solid. ¹H NMR (500 MHZ, DMSO-d₆) δ 12.46 (s, 1H), 10.27 (s, 1H), 9.83 (d, J=7.5 Hz, 1H), 8.99 (s, 1H), 8.67 (d, J=2.0 Hz, 1H), 8.59 (s, 1H), 8.32 (d, J=2.5 Hz, 1H), 7.98 (d, J=8.0 Hz, 2H), 7.62 (d, J=8.5 Hz, 2H), 7.45 (d, J=8.5 Hz, 1H), 7.21 (d, J=7.0 Hz, 1H), 7.03-7.00 (m, 1H), 4.40 (s, 1H), 2.36 (s, 3H). MS (ESI) m/z 446.0 [M+H]⁺

### Example 35. Synthesis of N-(3-((3-(9H-purin-6-yl)pyridin-2-yl)amino)-4-methylphenyl)-2-(oxetan-3-yl)isonicotinamide (Compound I-32)

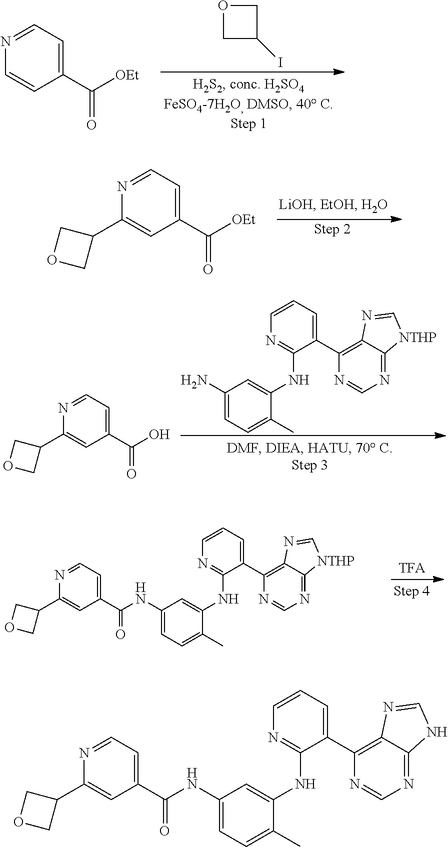

Step 1. Hydrogen peroxide (30% in water; 0.9 mL, 9.0 mmol) was added dropwise over 2 minutes to a stirred solution of ethyl isonicotinate (453 mg, 3.0 mmol), concentrated sulfuric acid (321 uL, 6.0 mmol), 3-iodooxetane (1104 mg, 6.0 mmol) and iron(II) sulfate heptahydrate (240 mg, 0.9 mmol) in dimethyl sulfoxide (10 mL) at 40° C. After 1-2 min another portion of iron (II) sulfate heptahydrate (240 mg, 0.9 mmol) was added and the mixture was stirred at 40° C. for 0.5 h. More hydrogen peroxide (0.9 mL, 9.0 mmol) and iron(II) sulfate heptahydrate (240 mg, 0.9 mmol) were added, and the mixture was stirred at 40° C. for 1 h. The mixture was diluted with ethyl acetate (50 mL) and washed with sodium thiosulfate (20 mL). The mixture was poured into saturated sodium bicarbonate aqueous solution (5 mL) and brine (100 mL). The aqueous and organic layers were separated and the organic layer was washed with brine (3×20 mL), dried over sodium sulfate, filtered and concentrated to leave a crude oil. The oil was purified by silica gel column chromatography (ethyl acetate/petroleum ether=1/3) to give ethyl 2-(oxetan-3-yl)isonicotinate (200 mg, 0.97 mmol, 32%) as an oil. MS (ESI) m/z 208.1 [M+H]⁺

Step 2. To a mixture of ethyl 2-(oxetan-3-yl)isonicotinate (200 mg, 0.97 mmol) in ethanol (10 mL) and water (5 mL) was added lithium hydroxide (122 mg, 2.90 mmol) at room temperature. Then the reaction mixture was stirred at room temperature for 2 h. The reaction mixture was adjusted to pH=6 with 1 N hydrochloric acid. The mixture was washed with ethyl acetate (50 mL). The aqueous phase was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 2-(oxetan-3-yl)isonicotinic acid (100 mg, 0.56 mmol, 59%) as a white solid. MS (ESI) m/z 180.1[M+H]⁺

Step 3. To a mixture of 2-(oxetan-3-yl)isonicotinic acid (40 mg, 0.22 mmol) in N,N-dimethylformamide (3 mL) was added N,N-diisopropylethylamine (86 mg, 0.67 mmol, 0.11 mL), 6-methyl-N1-(3-(9-(tetrahydro-2H-pyran-2-yl)-9H-purin-6-yl)pyridin-2-yl)benzene-1,3-diamine (90 mg, 0.22 mmol) and 1-[bis(dimethylamino)methylene]-1H-1,2,3-triazolo[4,5-b]pyridinium 3-oxid hexafluorophosphate (127 mg, 0.34 mmol). The mixture was stirred at 70° C. for 2 h. The mixture was diluted with ethyl acetate (50 mL), washed with saturated aqueous sodium bicarbonate solution (5 mL) and brine (20 mL×3), dried over sodium sulfate, filtered and concentrated. The residue was purified by silica gel column chromatography, eluting with ethyl acetate to give N-(4-methyl-3-((3-(9-(tetrahydro-2H-pyran-2-yl)-9H-purin-6-yl)pyridin-2-yl)amino)phenyl)-2-(oxetan-3-yl)isonicotinamide (100 mg, 0.18 mmol, 80%) as a white solid. MS (ESI) m/z 563.0 [M+H]⁺

Step 4. A mixture of N-(4-methyl-3-((3-(9-(tetrahydro-2H-pyran-2-yl)-9H-purin-6-yl)pyridin-2-yl)amino)phenyl)-2-(oxetan-3-yl)isonicotinamide (90 mg, 0.16 mmol) in trifluoroacetic acid (4 mL) was stirred at room temperature for 2 h. The reaction was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-2-(oxetan-3-yl)isonicotinamide (6.7 mg, 0.014 mmol, 4%) as a yellow solid. ¹H NMR (500 MHZ, DMSO-d₆) δ 2.44 (s, 3H), 4.50-4.55 (m, 1H), 4.85 (t, J=6.5 Hz, 2H), 4.93 (q, J=6.0 Hz, 2H), 7.04 (q, J=4.5 Hz, 1H), 7.24 (d, J=8.0 Hz, 1H), 7.45 (d, J=8.5 Hz, 1H), 7.77 (d, J=5.0 Hz, 1H), 7.82 (s, 1H), 8.34-8.36 (m, 1H), 8.68 (s, 1H), 8.72 (s, 1H), 8.79 (d, J=5.0 Hz, 1H), 9.08 (s, 1H), 9.82 (s, 1H), 10.44 (s, 1H), 12.34 (s, 1H), 13.85 (s, 1H). MS (ESI) m/z 479.1 [M+H]⁺

### Example 36. Synthesis of N-(3-((3-(9H-purin-6-yl)pyridin-2-yl)amino)-4-methylphenyl)-3-chloro-4-(trifluoromethyl)benzamide (Compound I-33)

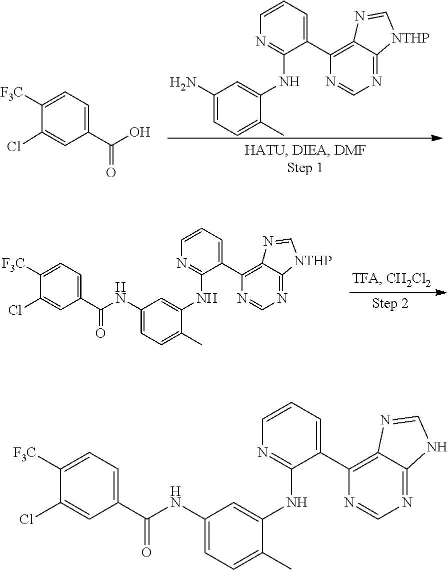

Step 1. A solution of 3-chloro-4-(trifluoromethyl)benzoic acid (50 mg, 0.22 mmol), 6-methyl-N1-(3-(9-(tetrahydro-2H-pyran-2-yl)-9H-purin-6-yl)pyridin-2-yl)benzene-1,3-diamine (107 mg, 0.27 mmol), 1-[bis(dimethylamino)methylene]-1H-1,2,3-triazolo[4,5-b]pyridinium 3-oxid hexafluorophosphate (83 mg, 0.24 mmol) and N,N-diisopropyl ethylamine (44 mg, 0.44 mmol) in N,N-dimethylformamide (2.0 mL) was stirred at room temperature for 16 h. The reaction mixture was diluted with ethyl acetate (80 mL) and washed with water (20 mL×3) and brine (30 mL), dried over sodium sulfate, filtered and concentrated. The residue was purified by flash chromatography (petroleum ether/ethyl acetate=2/3) to give 3-chloro-N-(4-methyl-3-((3-(9-(tetrahydro-2H-pyran-2-yl)-9H-purin-6-yl)pyridin-2-yl)amino)phenyl)-4-(trifluoromethyl)-benzamide (50 mg, 0.08 mmol, 8%) as a yellow solid. MS (ESI) m/z 607.8 [M+H]⁺

Step 2. A solution of 3-chloro-N-(4-methyl-3-((3-(9-(tetrahydro-2H-pyran-2-yl)-9H-purin-6-yl)pyridin-2-yl)amino)phenyl)-4-(trifluoromethyl)benzamide (50 mg, 0.08 mmol) in trifluoroacetic acid/dichloromethane (3 mL/3 mL) was stirred at room temperature for 1 h. The mixture was concentrated, the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-4-ethynylbenzamide (5.9 mg, 0.01 mmol, 14%) as a yellow solid. ¹H NMR (400 MHz, DMSO-d₆) δ 12.34 (s, 1H), 10.50 (s, 1H), 9.80 (s, 1H), 9.08 (s, 1H), 8.71 (s, 1H), 8.70 (d, J=2.0 Hz, 1H), 8.37-8.35 (m, 1H), 8.28 (s, 1H), 8.10 (d, J=8.0 Hz, 2H), 8.04 (d, J=8.4 Hz, 1H), 7.48 (dd, J=8.4 Hz, 2.0 Hz, 1H), 7.25 (d, J=8.4 Hz, 1H), 7.04 (dd, J=8.0 Hz, 4.8 Hz, 1H), 2.45 (s, 3H). MS (ESI) m/z 523.8 [M+H]⁺

### Example 37. Synthesis of 3-((3-(9H-purin-6-yl)pyridin-2-yl)amino)-4-methyl-N-(4-(trifluoromethyl)pyridin-2-yl)benzamide (Compound I-34)

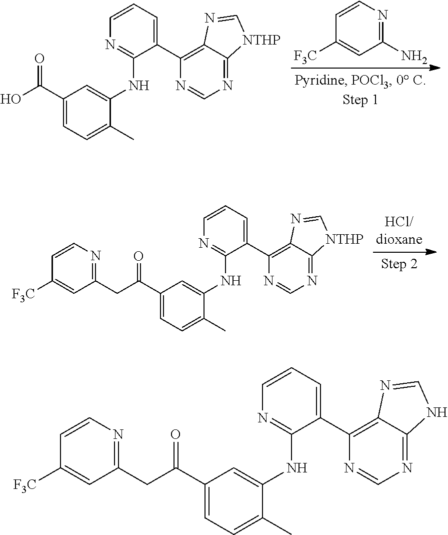

Step 1. To an ice-cooled solution of 4-methyl-3-((3-(9-(tetrahydro-2H-pyran-2-yl)-9H-purin-6-yl)pyridin-2-yl)amino)benzoic acid (100 mg, 0.23 mmol) and 4-(trifluoromethyl)pyridin-2-amine (112 mg, 0.69 mmol) in dry pyridine (4 mL) was added dropwise phosphorus oxychloride (101 mg, 0.66 mmol). The mixture was stirred at 0° C. for 2 h. The mixture was diluted with ethyl acetate (50 mL) and washed with saturated sodium bicarbonate solution (25 mL) and brine (25 mL). The organic layer was dried with sodium sulfate, filtered and concentrated. The residue was purified by flash chromatography (ethyl acetate in petroleum ether from 10% to 60%) to give 4-methyl-3-((3-(9-(tetrahydro-2H-pyran-2-yl)-9H-purin-6-yl)pyridin-2-yl)amino)-N-(4-(trifluoromethyl)pyridin-2-yl)benzamide (50 mg, 0.087 mmol, 38%) as yellow solid. MS (ESI) m/z 574.9 [M+H]⁺

Step 2. To a solution of 4-methyl-3-((3-(9-(tetrahydro-2H-pyran-2-yl)-9H-purin-6-yl)pyridin-2-yl)amino)-N-(4-(trifluoromethyl)pyridin-2-yl)benzamide (50 mg, 0.087 mmol) in dioxane (10 mL) was added hydrogen chloride solution (4 M in dioxane, 2 mL) at 25° C., and the mixture was stirred at 25° C. for 2 h. After completed, the reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 3-((3-(9H-purin-6-yl)pyridin-2-yl)amino)-4-methyl-N-(4-(trifluoromethyl)pyridin-2-yl)benzamide (17.2 mg, 0.035 mmol, 40%) as a yellow solid. ¹H NMR (400 MHZ, DMSO-d₆) δ 12.46 (s, 1H), 11.15 (s, 1H), 9.81 (d, J=7.8 Hz, 1H), 9.09 (s, 1H), 9.04 (s, 1H), 8.71 (s, 1H), 8.67 (d, J=5.0 Hz, 1H), 8.57 (s, 1H), 8.38 (d, J=2.9 Hz, 1H), 7.69 (d, J=7.8 Hz, 1H), 7.53 (d, J=4.8 Hz, 1H), 7.41 (d, J=8.0 Hz, 1H), 7.07 (dd, J=7.8, 4.7 Hz, 1H), 2.54 (s, 3H). MS (ESI) m/z 491.0 [M+H]⁺

### Example 38. Synthesis of 3-((3-(9H-purin-6-yl)pyridin-2-yl)amino)-4-methyl-N-(2-(trifluoromethyl)pyridin-4-yl)benzamide (Compound I-35)

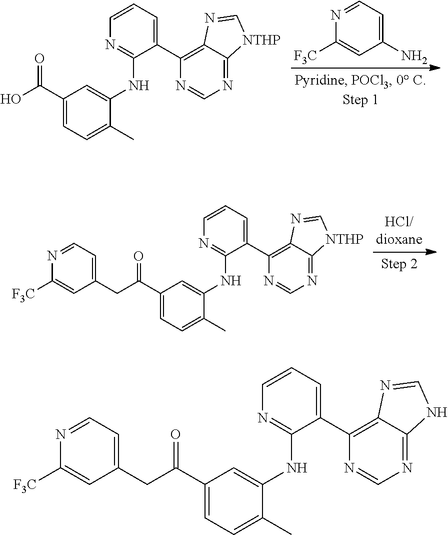

Step 1. To a mixture of 4-methyl-3-((3-(9-(tetrahydro-2H-pyran-2-yl)-9H-purin-6-yl)pyridin-2-yl)amino)benzoic acid (300 mg, 0.70 mmol) and 2-(trifluoromethyl)pyridin-4-amine (226 mg, 1.40 mmol) in dry pyridine (4 mL) was added phosphorus oxychloride (320 mg, 2.09 mmol), and the mixture was stirred at room temperature for 2 h. The mixture was diluted with ethyl acetate (30 mL), washed with saturated aqueous sodium bicarbonate solution (10 mL) and brine (25 mL), dried over sodium sulfate, filtered, and concentrated. The residue was purified by flash chromatography (6% methanol in dichloromethane) to give 4-methyl-3-((3-(9-(tetrahydro-2H-pyran-2-yl)-9H-purin-6-yl)pyridin-2-yl)amino)-N-(2-(trifluoromethyl)pyridin-4-yl)-benzamide (180 mg, 0.31 mmol, 45%) as a brown solid. MS (ESI) m/z 575.1 [M+H]⁺

Step 2. A solution of 4-methyl-3-((3-(9-(tetrahydro-2H-pyran-2-yl)-9H-purin-6-yl)pyridin-2-yl)amino)-N-(2-(trifluoromethyl)pyridin-4-yl)benzamide (50 mg, 0.09 mmol) in hydrogen chloride solution (4 M in dioxane, 6 mL) was stirred at room temperature for 3 h. The mixture was concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 3-((3-(9H-purin-6-yl)pyridin-2-yl)amino)-4-methyl-N-(2-(trifluoromethyl)pyridin-4-yl)benzamide (4.5 mg, 0.009 mmol, 10%) as a yellow solid. ¹H NMR (400 MHZ, DMSO-d₆) δ 13.86 (s, 1H), 12.48 (s, 1H), 10.86 (s, 1H), 9.82 (s, 1H), 9.10 (s, 1H), 9.01 (s, 1H), 8.73 (s, 1H), 8.66 (d, J=5.7 Hz, 1H), 8.38 (m, J=2.9 Hz, 1H), 8.35 (s, 1H), 8.08 (m, J=5.6 Hz, 1H), 7.63 (m, J=8.0 Hz, 1H), 7.47 (d, J=7.9 Hz, 1H), 7.08 (m, J=7.8, 4.7 Hz, 1H), 2.56 (s, 3H). MS (ESI) m/z 491.1 [M+H]⁺

### Example 39. Synthesis of N-(3-((3-(9H-purin-6-yl)pyridin-2-yl)amino)-4-methylphenyl)-4-chloro-3-ethynylbenzamide (Compound I-36)

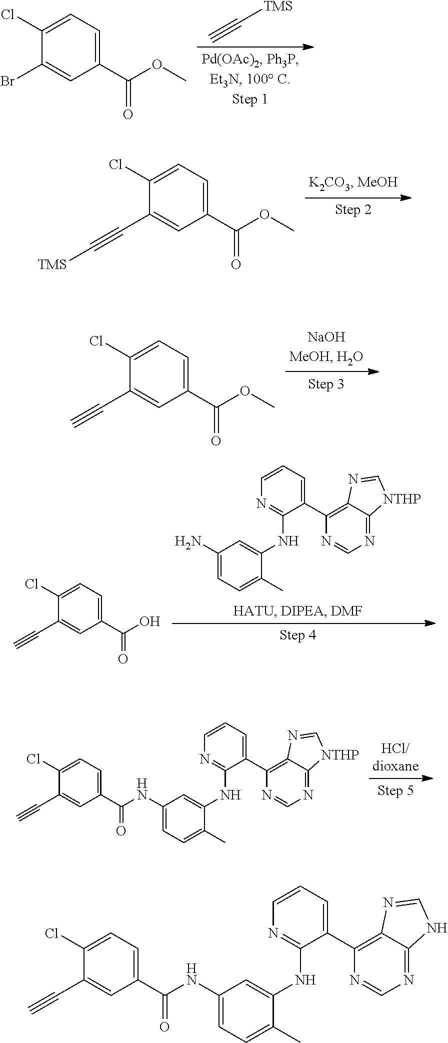

Step 1. A solution of methyl 3-bromo-4-chlorobenzoate (400 mg, 1.6 mmol), ethynyltrimethylsilane (173 mg, 1.76 mmol), palladium acetate (36 mg, 0.16 mmol) and triphenylphosphine (84 mg, 0.32 mmol) in triethylamine (5 mL) was stirred at 100° C. for 16 h. The reaction mixture was cooled and concentrated. The residue was diluted with ethyl acetate (50 mL) and washed with water (50 mL). The organic phase was washed with brine (50 mL), dried over sodium sulfate, filtered, and concentrated. The residue was purified by flash chromatography (petroleum ether/ethyl acetate=10/1) to give methyl 4-chloro-3-((trimethylsilyl)ethynyl)benzoate (395 mg, 1.48 mmol, 93%) as a yellow solid. ¹H NMR (500 MHz, CDCl₃) δ 8.16 (d, J=2.1 Hz, 1H), 7.93-7.83 (m, 1H), 7.46 (d, J=8.4 Hz, 1H), 3.92 (d, J=5.6 Hz, 3H), 0.31-0.26 (m, 9H).

Step 2. A solution of methyl 4-chloro-3-((trimethylsilyl)ethynyl)benzoate (395 mg, 1.48 mmol) in methanol (10 mL) was cooled to 0° C. and potassium carbonate (20 mg, 0.148 mmol) was added. The reaction mixture was allowed to warm to room temperature and was stirred for 0.5 h. The reaction mixture was concentrated and purified by flash chromatography (ethyl acetate in petroleum ether from 1% to 10%) to afford methyl 4-chloro-3-ethynylbenzoate (240 mg, 1.23 mmol, 83%) as a white solid. ¹H NMR (500 MHz, CDCl₃) δ 8.20 (d, J=2.1 Hz, 1H), 7.93 (dt, J=5.4, 2.7 Hz, 1H), 7.48 (d, J=8.4 Hz, 1H), 3.93 (s, 3H), 3.42 (s, 1H). MS (ESI) m/z 195.1 [M+H]⁺

Step 3. To a solution of methyl 4-chloro-3-ethynylbenzoate (240 mg, 1.23 mmol) in methanol (6 mL) were added sodium hydroxide (246 mg, 6.15 mmol) and water (6 mL), and the mixture was stirred at 25° C. for 3 h. The solvent was evaporated under reduced pressure, and the residue was diluted with water (10 mL). The mixture was adjusted to pH 3 by adding 1 N hydrochloric acid slowly. The resulting white precipitate was collected by filtration and washed with water (5 mL) to give 4-chloro-3-ethynylbenzoic acid (200 mg, 1.1 mmol, 89%) as a yellow solid. ¹H NMR (400 MHZ, DMSO) δ14.06 (s, 1H), 8.69 (s, 2H).

Step 4. A solution of 6-methyl-N1-(3-(9-(tetrahydro-2H-pyran-2-yl)-9H-purin-6-yl)pyridin-2-yl)benzene-1,3-diamine (116 mg, 0.29 mmol), 4-chloro-3-ethynylbenzoic acid (60 mg, 0.33 mmol), 1-[bis(dimethylamino)methylene]-1H-1,2,3-triazolo[4,5-b]pyridinium 3-oxid hexafluorophosphate (190 mg, 0.5 mmol) and N,N-diisopropylethylamine (170 mg, 1.32 mmol) in N,N-dimethylformamide (4 mL) was stirred at 25° C. for 1 h. The reaction mixture was diluted with ethyl acetate (50 mL), then washed with water (25 mL) and brine (25 mL×3). The organic layer was dried over sodium sulfate, filtered, and concentrated under reduced pressure. The residue was purified by column chromatography (ethyl acetate/petroleum ether=50/100) to give 4-chloro-3-ethynyl-N-(4-methyl-3-((3-(9-(tetrahydro-2H-pyran-2-yl)-9H-purin-6-yl)pyridin-2-yl)amino)phenyl)benzamide (60 mg, 0.11 mmol, 38%) as a yellow solid. MS (ESI) m/z 563.8 [M+H]⁺

Step 5. To a solution of 4-chloro-3-ethynyl-N-(4-methyl-3-((3-(9-(tetrahydro-2H-pyran-2-yl)-9H-purin-6-yl)pyridin-2-yl)amino)phenyl)benzamide (60 mg, 0.11 mmol) in dioxane (10 mL) was added hydrogen chloride solution (4 M in dioxane, 2 mL) at 25° C., and stirred at 25° C. for 1 h. After completed, the reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-4-chloro-3-ethynylbenzamide (30.2 mg, 0.06 mmol, 55%) as a yellow solid. ¹H NMR (400 MHZ, DMSO-d₆) δ 13.86 (s, 1H), 12.30 (s, 1H), 10.34 (s, 1H), 9.79 (s, 1H), 9.09 (s, 1H), 8.70 (d, J=14.0 Hz, 1H), 8.67 (s, 1H), 8.36 (d, J=3.1 Hz, 1H), 8.23 (d, J=1.8 Hz, 1H), 8.00 (d, J=8.5 Hz, 1H), 7.73 (d, J=8.4 Hz, 1H), 7.48 (d, J=8.1 Hz, 1H), 7.23 (d, J=8.3 Hz, 1H), 7.04 (dd, J=7.8, 4.7 Hz, 1H), 4.72 (s, 1H), 2.44 (s, 3H). MS (ESI) m/z 480.1 [M+H]⁺

### Example 40. Synthesis of N-(3-((3-(9H-purin-6-yl)pyridin-2-yl)amino)-4-methylphenyl)-4-chloro-3-(2-cyanopropan-2-yl)benzamide (Compound I-37)

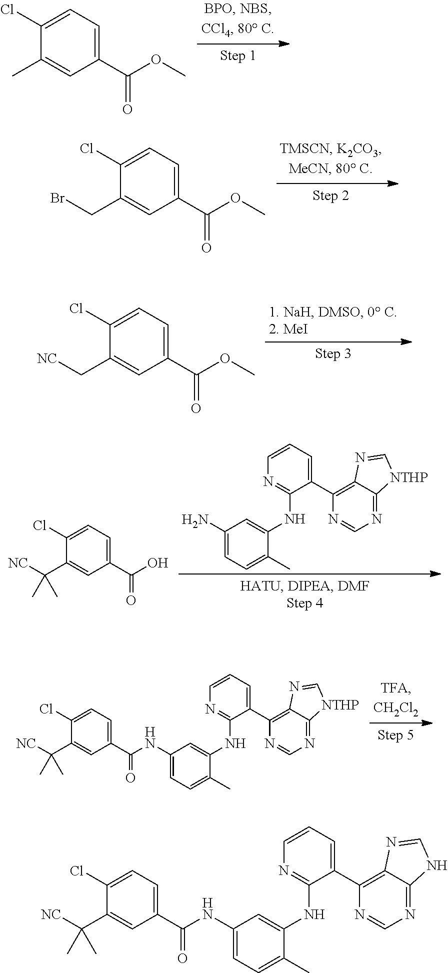

Step 1. To a solution of methyl 4-chloro-3-methylbenzoate (1 g, 5.43 mmol) in carbon tetrachloride (15 mL) were added benzoyl peroxide (121 mg, 0.5 mmol), and N-bromosuccinimide (1.063 g, 5.97 mmol) at room temperature. The reaction mixture was stirred at 80° C. for 3 h. The reaction mixture was diluted with water (50 mL) and extracted with ethyl acetate (50 mL×3). The combined organic layer was dried over sodium sulfate, filtered, and concentrated to give methyl 3-(bromomethyl)-4-chlorobenzoate (950 mg, crude) as a yellow oil. MS (ESI) m/z 265.0 [M+H]⁺

Step 2. A solution of methyl 3-(bromomethyl)-4-chlorobenzoate (950 mg, crude), trimethylsilyl cyanide (430 mg, 4.3 mmol) and potassium carbonate (1.49 g, 10.8 mmol) in acetonitrile (20 mL) was stirred 80° C. for 16 h. The reaction mixture was diluted with water (60 mL) and extracted with ethyl acetate (50 mL×3). The combined organic layer was dried over sodium sulfate, filtered, and concentrated. The residue was purified by silica gel column chormatography, eluting with petroleum ether/ethyl acetate=5/1, to give methyl 4-chloro-3-(cyanomethyl)benzoate (530 mg, 2.5 mmol, 70%) as a colorless oil. MS (ESI) m/z 210.1 [M+H]⁺

Step 3. To a solution of methyl 4-chloro-3-(cyanomethyl)benzoate (100 mg, 0.48 mmol) in dimethyl sulfoxide (5 mL) was added sodium hydride (60% in mineral oil, 57 mg, 1.43 mmol) at 0° C. After stirring for 30 minutes, iodomethane (204 mg, 1.44 mmol) was added. The reaction mixture was stirred at room temperature for 16 h. The reaction mixture was diluted with water (20 mL) at 0° C. and extracted with ethyl acetate (30 mL×3). The combined organic layer was dried over sodium sulfate, filtered, and concentrated to give 4-chloro-3-(2-cyanopropan-2-yl)benzoic acid (25 mg, 0.11 mmol, 23%) as a white solid. MS (ESI) m/z 224.1 [M+H]⁺

Step 4. A solution of 4-chloro-3-(2-cyanopropan-2-yl)benzoic acid (25 mg, 0.11 mmol), 6-methyl-N1-(3-(9-(tetrahydro-2H-pyran-2-yl)-9H-purin-6-yl)pyridin-2-yl)benzene-1,3-diamine (50 mg, 0.12 mmol), N,N-diisopropylethylamine (38.7 mg, 0.3 mmol) and 1-[bis(dimethylamino)methylene]-1H-1,2,3-triazolo[4,5-b]pyridinium 3-oxid hexafluorophosphate (76 mg, 0.2 mmol) in N,N-dimethylformamide (5 mL) was stirred at 25° C. for 3 h. The reaction was quenched with water (25 mL) and extracted with ethyl acetate (15 mL×3). The organic layer was concentrated to give 4-chloro-3-(2-cyanopropan-2-yl)-N-(4-methyl-3-((3-(9-(tetrahydro-2H-pyran-2-yl)-9H-purin-6-yl)pyridin-2-yl)amino)phenyl)benzamide (35 mg, 0.06 mmol, 55%) as a yellow solid. MS (ESI) m/z 607.3 [M+H]⁺

Step 5. To a solution of 4-chloro-3-(2-cyanopropan-2-yl)-N-(4-methyl-3-((3-(9-(tetrahydro-2H-pyran-2-yl)-9H-purin-6-yl)pyridin-2-yl)amino)phenyl)benzamide (35 mg, 0.06 mmol) in dichloromethane (5 mL) were added trifluoroacetic acid (1 mL). The mixture was stirred at room temperature for 2 h. The mixture was concentrated in vacuo. The residue was diluted with saturated aqueous sodium bicarbonate solution (20 mL) and extracted with ethyl acetate (20 mL×3). The combined organic layers were concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-4-chloro-3-(2-cyanopropan-2-yl)benzamide (3.5 mg, 0.0067 mmol, 12%) as a yellow solid. ¹H NMR (400 MHZ, DMSO-d₆) δ 12.57-12.26 (m, 1H), 10.35 (s, 1H), 9.97-9.58 (m, 1H), 9.05 (s, 1H), 8.65 (s, 2H), 8.40-8.22 (m, 1H), 8.03 (s, 2H), 7.72 (d, J=8.2 Hz, 1H), 7.52-7.37 (m, 1H), 7.25 (s, 1H), 7.16-6.93 (m, 1H), 2.44 (s, 3H), 1.88 (s, 6H). MS (ESI) m/z 523.0 [M+H]⁺

### Example 41. Synthesis of N-(3-((3-(9H-purin-6-yl)pyridin-2-yl)amino)-4-methylphenyl)-4-chloro-3-(1-cyanocyclopropyl)benzamide (Compound I-38)

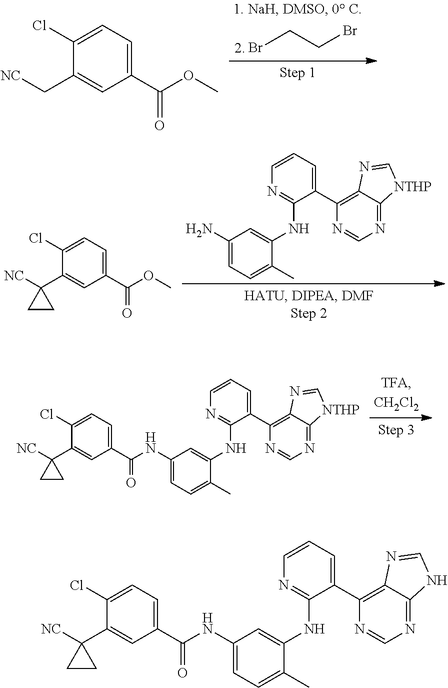

Step 1. To a solution of methyl 4-chloro-3-(cyanomethyl)benzoate (100 mg, 0.48 mmol) in dimethyl sulfoxide (10 mL) was added sodium hydride (60% in mineral oil, 57 mg, 1.43 mmol) at 0° C. After stirring for 30 minutes, 1,2-dibromoethane (135.4 mg, 0.72 mmol) was added. The reaction mixture was stirred at room temperature for 3 h. The reaction mixture was diluted with water (20 mL) at 0° C., and extracted with ethyl acetate (30 mL×3). The combined organic layer was dried over sodium sulfate, filtered, and concentrated to give 4-chloro-3-(1-cyanocyclopropyl)benzoic acid (30 mg, 0.14 mmol, 28%) as a white solid. MS (ESI) m/z 222.0[M+H]⁺

Step 2. A solution of 4-chloro-3-(1-cyanocyclopropyl)benzoic acid (30 mg, 0.14 mmol), 6-methyl-N1-(3-(9-(tetrahydro-2H-pyran-2-yl)-9H-purin-6-yl)pyridin-2-yl)benzene-1,3-diamine (50 mg, 0.12 mmol), N,N-diisopropylethylamine (38.7 mg, 0.3 mmol) and 1-[bis(dimethylamino)methylene]-1H-1,2,3-triazolo[4,5-b]pyridinium 3-oxid hexafluorophosphate (76 mg, 0.2 mmol) in N,N-dimethylformamide (5 mL) was stirred at 25° C. for 3 h. The reaction was quenched with water (25 mL) and extracted with ethyl acetate (15 mL×3). The organic layer was concentrated to give 4-chloro-3-(1-cyanocyclopropyl)-N-(4-methyl-3-((3-(9-(tetrahydro-2H-pyran-2-yl)-9H-purin-6-yl)pyridin-2-yl)amino)phenyl)benzamide (55 mg, 0.09 mmol, 65%) as a yellow solid. MS (ESI) m/z 605.1 [M+H]⁺

Step 3. To a solution of 4-chloro-3-(2-cyanopropan-2-yl)-N-(4-methyl-3-((3-(9-(tetrahydro-2H-pyran-2-yl)-9H-purin-6-yl)pyridin-2-yl)amino)phenyl)benzamide (55 mg, 0.09 mmol) in dichloromethane (5 mL) were added trifluoroacetic acid (1 mL). The mixture was stirred at room temperature for 2 h and then concentrated in vacuo. The residue was diluted with saturated aqueous sodium bicarbonate solution (20 mL) and extracted with ethyl acetate (20 mL×3). The combined organic phases were concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-4-chloro-3-(1-cyanocyclopropyl)benzamide (6.8 mg, 0.013 mmol, 14%) as a yellow solid. ¹H NMR (400 MHZ, DMSO-d₆) δ 12.51-12.24 (m, 1H), 10.30 (s, 1H), 9.92-9.54 (m, 1H), 9.04 (s, 1H), 8.66 (s, 2H), 8.34 (s, 1H), 8.15-7.89 (m, 2H), 7.72 (d, J=8.4 Hz, 1H), 7.54-7.35 (m, 1H), 7.24 (d, J=8.4 Hz, 1H), 7.13-6.85 (m, 1H), 2.44 (s, 3H), 1.81 (m, 2H), 1.55 (m, 2H). MS (ESI) m/z 521.0 [M+H]⁺

### Example 42. Synthesis of N-(3-((3-(9H-purin-6-yl)pyridin-2-yl)amino)-4-methylphenyl)-5-chloro-4-cyanopicolinamide (Compound I-39)

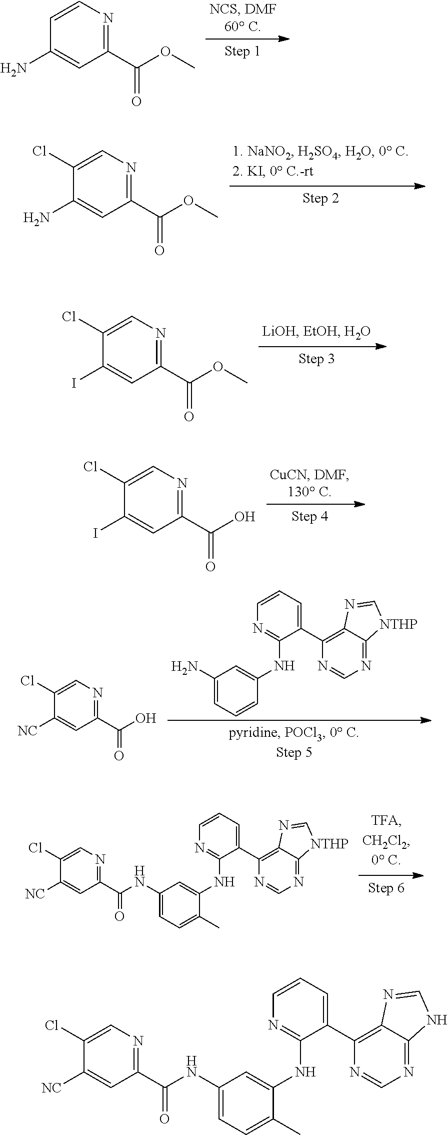

Step 1. N-Chlorosuccinimide (5.3 g, 39.5 mmol) was added to a solution of methyl 4-aminopicolinate (6.0 g, 39.5 mmol) in N,N-dimethylformamide (50 mL) and the reaction mixture was stirred at 60° C. for 16 h. The mixture was diluted with ethyl acetate (50 mL), and then the organic layer was washed with water (25 mL) and brine (25 mL), dried over sodium sulfate, filtered, and concentrated. The residue was purified by flash chromatography (petroleum ether/ethyl acetate=1/1) to give methyl 4-amino-5-chloropicolinate (2 g, 10.75 mmol, 41%) as a white solid. MS (ESI) m/z 187.1 [M+H]⁺

Step 2. A mixture of methyl 4-amino-5-chloropicolinate (700 mg, 3.76 mmol) in water (4 mL) and sulfuric acid (16 mL) was cooled to 0° C. To this mixture was added a solution of sodium nitrite (1.04 g, 15.1 mmol) in water (3 mL) and the mixture was stirred at 0° C. for 30 minutes. A solution of potassium iodide (2.50 mg, 15.1 mmol) in water (4 mL) was slowly added at 0° C. and the reaction was stirred at room temperature for 10 minutes. The reaction was diluted with water (50 mL) and extracted with ethyl acetate. The organic layer was washed with saturated sodium thiosulfate solution (50 mL) and brine (40 mL), dried over sodium sulfate, filtered and concentrated. The residue was purified by flash chromatography (petroleum ether/ethyl acetate=10/1) to give methyl 5-chloro-4-iodopicolinate (150 mg, 0.51 mmol, 14%) as a yellow solid. MS (ESI) m/z 298.0 [M+H]⁺

Step 3. To a mixture of methyl 5-chloro-4-iodopicolinate (150 mg, 0.74 mmol) in ethanol (6 mL) and water (3 mL) was added lithium hydroxide (42 mg, 1.01 mmol) at room temperature. The reaction mixture was stirred at room temperature for 1 h and then concentrated under reduced pressure to remove ethanol. The aqueous phase was acidified with 1 N hydrochloric acid until precipitation occurs. The solid was filtered and washed through with water (5 mL) to give 5-chloro-4-iodopicolinic acid (80 mg, 0.28 mmol, 57%) as a white solid. MS (ESI) m/z 283.8 [M+H]⁺

Step 4. Copper cyanide (31 mg, 0.34 mmol) was added at room temperature to a stirred solution of 5-chloro-4-iodopicolinic acid (80 mg, 0.28 mol) in N,N-dimethylformamide (4 mL). After stirring at 130° C. for 16 h, the reaction mixture was cooled to room temperature. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 5-chloro-4-cyanopicolinic acid (40 mg, 0.22 mmol, 80%) as a yellow solid. MS (ESI) m/z 183.1 [M+H]⁺

Step 5. To a mixture of 5-chloro-4-cyanopicolinic acid (35 mg, 0.191 mmol) and 6-methyl-N1-(3-(9-(tetrahydro-2H-pyran-2-yl)-9H-purin-6-yl)pyridin-2-yl)benzene-1,3-diamine (77 mg, 0.191 mmol) in dry pyridine (3 mL) was added dropwise phosphorus oxychloride (88 mg, 0.573 mmol). The mixture was stirred at 0° C. for 1 h under nitrogen. The mixture was extracted with ethyl acetate (50 mL×3). The combined organic layer was washed with brine (50 mL), dried over sodium sulfate, and filtered. The filtrate was concentrated under reduced pressure. The residue was purified by flash chromatography eluting with ethyl acetate to give 5-chloro-4-cyano-N-(4-methyl-3-((3-(9-(tetrahydro-2H-pyran-2-yl)-9H-purin-6-yl)pyridin-2-yl)amino)phenyl)-picolinamide (20 mg, 0.035 mmol, 11%) as a yellow solid. MS (ESI) m/z 565.8 [M+H]⁺

Step 6. A solution of 5-chloro-4-cyano-N-(4-methyl-3-((3-(9-(tetrahydro-2H-pyran-2-yl)-9H-purin-6-yl)pyridin-2-yl)amino)phenyl)picolinamide (0.015 g, 0.03 mmol) and trifluoroacetic acid (0.50 mL) in dichloromethane (2.0 mL) was stirred at 0° C. for 1 h. The solvent was removed and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-5-chloro-4-cyanopicolinamide (2.7 mg, 0.005 mmol, 19%) as a white solid. ¹H NMR (500 MHZ, DMSO-d₆): δ 12.34 (s, 1H), 10.61 (s, 1H), 9.86 (s, 1H), 9.08 (d, J=12 Hz, 2H), 8.80 (s, 1H), 8.71 (s, 1H), 8.59 (s, 1H), 8.35 (d, J=4.5 Hz, 1H), 7.51 (d, J=8.0 Hz, 1H), 7.25 (d, J=7.5 Hz, 1H), 7.03 (d, J=8.0 Hz, 1H), 2.36 (s, 3H). MS (ESI) m/z 482.1 [M+H]⁺

### Example 43. Synthesis of N-(5-((3-(9H-purin-6-yl)pyridin-2-yl)amino)-6-methylpyridin-3-yl)-4-chloro-3-cyanobenzamide (Compound I-40)

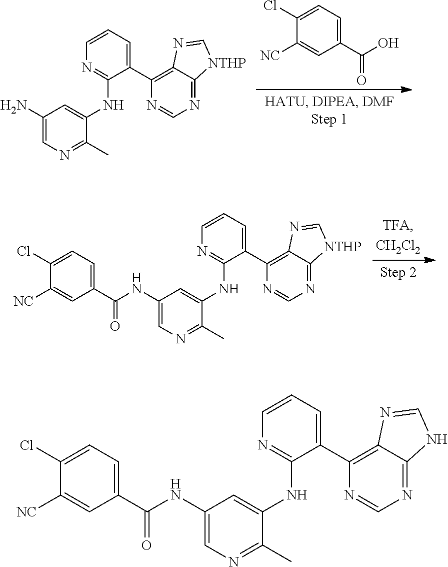

Step 1. A mixture of 4-chloro-3-cyanobenzoic acid (50 mg, 0.28 mmol), N,N-diisopropylethylamine (71 mg, 0.55 mmol) and 1-[bis(dimethylamino)methylene]-1H-1,2,3-triazolo[4,5-b]pyridinium 3-oxid hexafluorophosphate (84 mg, 0.22 mmol) in N,N-dimethylformamide (1.5 mL) was stirred at room temperature for 30 minutes, then 2-methyl-N3-(3-(9-(tetrahydro-2H-pyran-2-yl)-9H-purin-6-yl)pyridin-2-yl)pyridine-3,5-diamine (74 mg, 0.18 mmol) was added. The mixture was stirred at room temperature for 4 h. The reaction was diluted with water (10 mL) and extracted with ethyl acetate (10 mL×3). The combined organic layer was washed with brine (10 mL×2), dried over sodium sulfate, filtered, and concentrated. The residue was purified by flash chromatography (3% methanol in dichloromethane) to give 4-chloro-3-cyano-N-(6-methyl-5-((3-(9-(tetrahydro-2H-pyran-2-yl)-9H-purin-6-yl)pyridin-2-yl)amino)-pyridin-3-yl)benzamide (60 mg, 0.11 mmol, 58%) as a brown oil. MS (ESI) m/z 566.1 [M+H]⁺

Step 2. A mixture of 4-chloro-3-cyano-N-(6-methyl-5-((3-(9-(tetrahydro-2H-pyran-2-yl)-9H-purin-6-yl)pyridin-2-yl)amino)pyridin-3-yl)benzamide (60 mg, 0.11 mmol) in trifluoroacetic acid (2 mL) and dichloromethane (2 mL) was stirred at room temperature for 2 h. The mixture was concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(5-((3-(9H-purin-6-yl)pyridin-2-yl)amino)-6-methylpyridin-3-yl)-4-chloro-3-cyanobenzamide (5.9 mg, 0.012 mmol, 12%) as a yellow solid. ¹H NMR (400 MHZ, DMSO-d₆) δ 12.71 (s, 1H), 10.60 (s, 1H), 9.88 (d, J=7.6 Hz, 1H), 9.21 (d, J=2.0 Hz, 1H), 9.07 (s, 1H), 8.67 (s, 1H), 8.64-8.57 (m, 2H), 8.43-8.36 (m, 1H), 8.30 (m, J=8.5, 2.1 Hz, 1H), 7.95 (d, J=8.5 Hz, 1H), 7.11 (dd, J=7.8, 4.7 Hz, 1H), 2.70 (s, 3H). MS (ESI) m/z 482.1 [M+H]⁺

### Example 44. Synthesis of N-(3-((3-(9H-purin-6-yl)pyridin-2-yl)amino)-4-methylphenyl)-4-chloro-3-methoxybenzamide (Compound I-41)

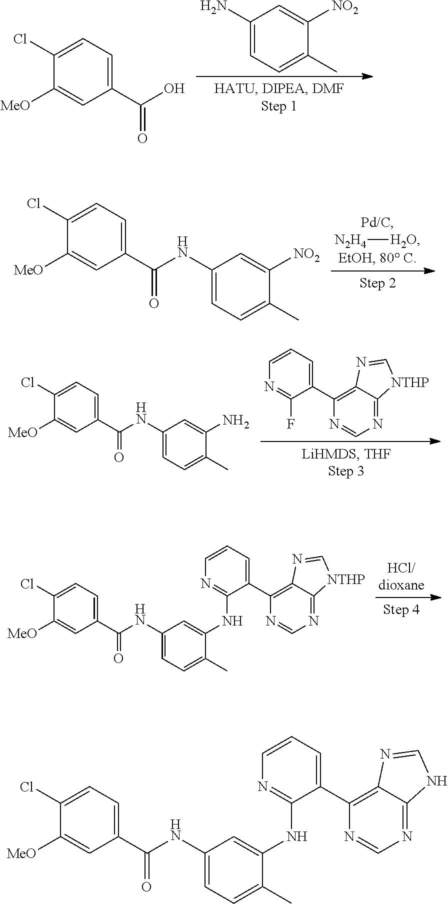

Step 1. A solution of 4-chloro-3-methoxybenzoic acid (180 mg, 0.968 mmol), 4-fluoro-3-nitroaniline (191 mg, 1.258 mmol), 1-[bis(dimethylamino)methylene]-1H-1,2,3-triazolo[4,5-b]pyridinium 3-oxid hexafluorophosphate (478 mg, 1.258 mmol) and N,N-diisopropylethylamine (354 mg, 2.904 mmol) in N,N-dimethylformamide (6 mL) was stirred at 25° C. for 16 h. Then water was added and the mixture was extracted with ethyl acetate. The organic layer was dried and concentrated. The crude residue was purified by silica gel column chromatography (petroleum ether/ethyl acetate from 50/1 to 10/1) to afford 4-chloro-3-methoxy-N-(4-methyl-3-nitrophenyl)benzamide (300 mg, 0.937 mmol, 96%) as a yellow solid. MS (ESI) m/z 321.0 [M+H]⁺

Step 2. A solution of 4-chloro-3-methoxy-N-(4-methyl-3-nitrophenyl)benzamide (300 mg, 0.937 mmol), hydrazine hydrate (234 mg, 4.687 mmol) and palladium (10% on carbon, 80 mg) in ethanol (10 mL) was stirred at 80° C. for 1 h. The mixture was concentrated. The crude residue was purified by silica gel column chromatography (dichloromethane/methanol from 50:1 to 10:1) to give N-(3-amino-4-methylphenyl)-4-chloro-3-methoxybenzamide (160 mg, 0.55 mol, 59%) as a yellow solid. MS (ESI) m/z 291.1 [M+H]⁺

Step 3. A solution of N-(3-amino-4-methylphenyl)-4-chloro-3-methoxybenzamide (90 mg, 0.310 mmol), 6-(2-fluoropyridin-3-yl)-9-(tetrahydro-2H-pyran-2-yl)-9H-purine (92 mg, 0.310 mmol) in tetrahydrofuran (5 mL) was stirred at 0° C. for 10 min. Then lithium hexamethyldisilazide (0.62 mL, 0.620 mmol) was added. The mixture was stirred at room temperature for 2 h, then diluted with ethyl acetate and washed with water. The organic layer was concentrated. The crude residue was purified by silica gel column chromatography (dichloromethane/methanol from 50:1 to 10:1) to give 4-chloro-3-methoxy-N-(4-methyl-3-((3-(9-(tetrahydro-2H-pyran-2-yl)-9H-purin-6-yl)pyridin-2-yl)amino)phenyl)benzamide (120 mg, 0.211 mmol, 68%) as a yellow solid. MS (ESI) m/z 570.2 [M+H]⁺

Step 4. A solution of 4-chloro-3-methoxy-N-(4-methyl-3-((3-(9-(tetrahydro-2H-pyran-2-yl)-9H-purin-6-yl)pyridin-2-yl)amino)phenyl)benzamide (120 mg, 0.211 mmol) and hydrochloric acid (4 M in dioxane, 0.52 mL, 2.076 mmol) in 1,4-dioxane (5 mL) was stirred at 25° C. for 1 h. The mixture was concentrated. The crude residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-4-chloro-3-methoxybenzamide (14.5 mg, 0.03 mmol, 14%) as a yellow solid. ¹H NMR (400 MHZ, DMSO-d₆) δ 12.832 (s, 1H), 10.223 (s, 1H), 9.947-9.965 (d, J=7.2 Hz, 1H), 8.821 (s, 1H), 8.104 (s, 1H), 8.313 (s, 1H), 8.257-8.274 (dd, J=5.2 Hz, 2.8 Hz, 1H), 7.683 (s, 1H), 7.587 (s, 2H), 7.397-7.418 (d, J=8.4 Hz, 1H), 7.199-7.219 (d, J=8.0 Hz, 1H), 6.960-6.991 (dd, J=8.0 Hz, 4.8 Hz, 1H), 3.964 (s, 3H), 2.458 (s, 3H). MS (ESI) m/z 486.3 [M+H]⁺

### Example 45. Synthesis of N-(3-((3-(9H-purin-6-yl)pyridin-2-yl)amino)-4-methylphenyl)-3-cyano-4-(trifluoromethyl)benzamide (Compound I-42)

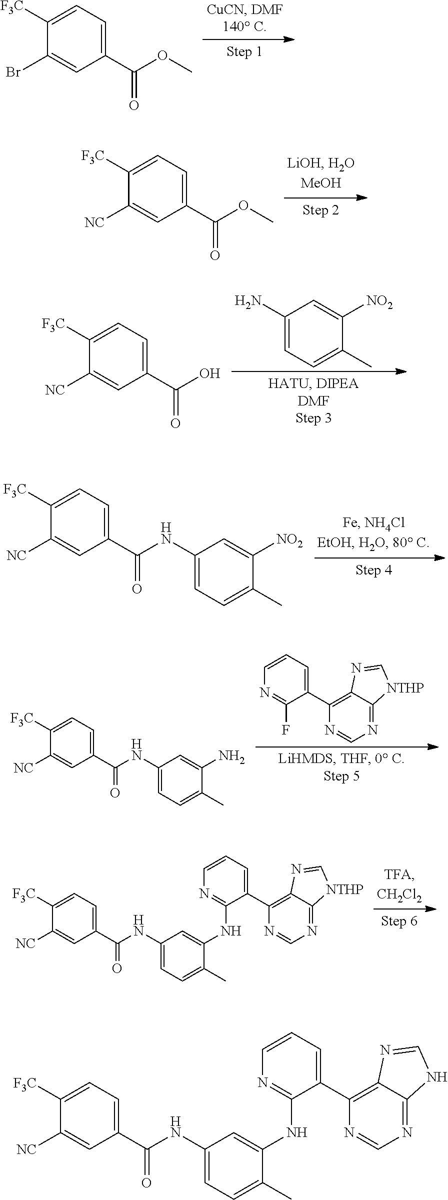

Step 1. Copper(I) cyanide (107 mg, 1.2 mmol) was added at room temperature to a stirred solution of methyl 3-bromo-4-(trifluoromethyl)benzoate (282 mg, 1.0 mol) in N,N-dimethylformamide (10 mL). The mixture was stirred at 140° C. for 16 h. Then the mixture was poured into water at 0° C. and diluted with ethyl acetate (30 mL). The organic layer was washed with water (20 mL×3) and brine (20 mL), dried over sodium sulfate, filtered, and concentrated. The residue was purified by flash chromatography (petroleum ether/ethyl acetate=1/4) to methyl 3-cyano-4-(trifluoromethyl)benzoate (200 mg, 0.87 mmol, 87%) as a yellow solid. MS (ESI) m/z 230.1 [M+H]⁺

Step 2. To a solution of methyl 3-cyano-4-(trifluoromethyl)benzoate (200 mg, 0.87 mmol) in methanol (5 mL)/water (2.5 mL) was added lithium hydroxide (42 mg, 1.74 mmol). The mixture was stirred at room temperature for 2 h. On completion, the mixture was concentrated. The residue was diluted with water (3 mL). The aqueous phase was acidified with 1 N hydrochloric acid until no more precipitates formed. The mixture was filtered. The solid was washed with water (10 mL) and dried to give 3-cyano-4-(trifluoromethyl)benzoic acid (170 mg, 0.79 mmol, 90%) as a white solid. MS (ESI) m/z 216.1 [M+H]⁺

Step 3. To a solution of 3-cyano-4-(trifluoromethyl)benzoic acid (170 mg, 0.79 mmol) in N,N-dimethylformamide (3 mL) was added 4-methyl-3-nitroaniline (133 mg, 0.87 mmol), 1-[bis(dimethylamino)methylene]-1H-1,2,3-triazolo[4,5-b]pyridinium 3-oxid hexafluorophosphate (380 mg, 0.95 mmol) and N,N-diisopropylethylamine (204 mg, 1.58 mmol). The mixture was stirred at room temperature for 2 h. The reaction mixture was poured into water (10 mL), and then diluted with ethyl acetate (20 mL). The organic layer was washed with water (20 mL×3) and brine (20 mL), dried over sodium sulfate, filtered and concentrated. The residue was purified by flash chromatography (petroleum ether/ethyl acetate=1/1) to give 3-cyano-N-(4-methyl-3-nitrophenyl)-4-(trifluoromethyl)benzamide (200 mg, 0.57 mmol, 72%) as a yellow solid. MS (ESI) m/z 350.0 [M+H]⁺

Step 4. A mixture of 3-cyano-N-(4-methyl-3-nitrophenyl)-4-(trifluoromethyl)benzamide (200 mg, 0.57 mmol), iron (128 mg, 2.28 mmol) and ammonium chloride (242 mg, 4.56 mmol) in ethanol (16 mL) and water (4 mL) was stirred at 80° C. for 1 h. After cooling to room temperature, the reaction was filtered through Celite. The filter pad was washed with ethyl acetate and the filtrate was washed with saturated sodium bicarbonate aqueous solution (50 mL) and brine (40 mL), dried over sodium sulfate, filtered, and concentrated to afford N-(3-amino-4-methylphenyl)-3-cyano-4-(trifluoromethyl)benzamide (160 mg, 0.50 mmol, 87%) as a yellow solid. MS (ESI) m/z 320.0 [M+H]⁺

Step 5. To a solution of N-(3-amino-4-methylphenyl)-3-cyano-4-(trifluoromethyl)-benzamide (160 mg, 0.50 mmol), 6-(2-fluoropyridin-3-yl)-9-(tetrahydro-2H-pyran-2-yl)-9H-purine (150 mg, 0.50 mmol) in tetrahydrofuran (10 mL) was added lithium hexamethyldisilazide (0.93 mL, 1.5 mmol, 1.6 M in tetrahydrofuran) at 0° C. slowly. The mixture was stirred at 0° C. for 1 h. The reaction was quenched with saturated ammonium chloride solution (3 mL) at 0° C. and extracted with dichloromethane (15 mL×3). The combined organic layer was washed with brine (30 mL), dried over sodium sulfate, and filtered. The filtrate was concentrated under reduced pressure. The crude residue was purified by flash chromatography (ethyl acetate/petroleum ether=1/1) to give 3-cyano-N-(4-methyl-3-((3-(9-(tetrahydro-2H-pyran-2-yl)-9H-purin-6-yl)pyridin-2-yl)amino)phenyl)-4-(trifluoromethyl)benzamide (120 mg, 0.20 mmol, 40%) as a yellow solid. MS (ESI) m/z 598.8 [M+H]⁺

Step 6. A mixture of 3-cyano-N-(4-methyl-3-((3-(9-(tetrahydro-2H-pyran-2-yl)-9H-purin-6-yl)pyridin-2-yl)amino)phenyl)-4-(trifluoromethyl)benzamide (120 mg, 0.2 mmol) in trifluoroacetic acid (3 mL)/dichloromethane (3 mL) was stirred at room temperature for 2 h. The reaction mixture was concentrated under reduced pressure to give a residue, which was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl) pyridin-2-yl)amino)-4-methylphenyl)-3-cyano-4-(trifluoromethyl)benzamide (24.8 mg, 0.05 mmol, 24%) as a yellow solid. ¹H NMR (400 MHZ, DMSO-d₆) δ 13.87 (s, 1H), 12.37 (s, 1H), 10.57 (s, 1H), 9.83 (d, J=5.2 Hz, 1H), 9.09 (s, 1H), 8.73 (s, 3H), 8.45 (d, J=8.8 Hz, 1H), 8.37 (dd, J=4.8, 1.6 Hz, 1H), 8.20 (d, J=8.4 Hz, 1H), 7.50 (dd, J=8.0, 1.6 Hz, 1H), 7.27 (d, J=8.4 Hz, 1H), 7.05 (dd, J=8.0, 4.8 Hz, 1H), 2.46 (s, 3H). MS (ESI) m/z 514.9 [M+H]⁺

### Example 46. Synthesis of N-(3-((3-(9H-purin-6-yl)pyridin-2-yl)amino)-4-methylphenyl)-3-cyano-4-methylbenzamide (Compound I-43)

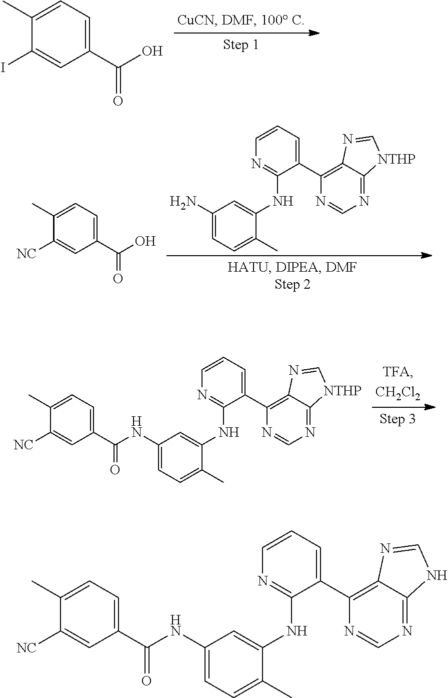

Step 1. 3-Iodo-4-methylbenzoic acid (262 mg, 1 mmol) and copper(I) cyanide (97 mg, 1.1 mmol) were dissolved in N,N-dimethylformamide (4 mL) and stirred at 100° C. for 16 h. The mixture was concentrated in vacuo. The residue was diluted with water (100 mL) and extracted with ethyl acetate (3×100 mL). The combined organic phases were concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to yield 3-cyano-4-methylbenzoic acid (50 mg, 0.31 mmol, 31%) as a white solid. MS (ESI) m/z 162 [M+H]⁺

Step 2. 3-Cyano-4-methylbenzoic acid (48 mg, 0.3 mmol), 6-methyl-N1-(3-(9-(tetrahydro-2H-pyran-2-yl)-9H-purin-6-yl)pyridin-2-yl)benzene-1,3-diamine (120 mg, 0.3 mmol), 1-[bis(dimethylamino)methylene]-1H-1,2,3-triazolo[4,5-b]pyridinium 3-oxid hexafluorophosphate (171 mg, 0.45 mmol) and N,N-diisopropylethylamine (77 mg, 0.6 mmol) were dissolved in N,N-dimethylformamide (4 mL) and stirred at room temperature for 4 h. The mixture was concentrated in vacuo. The residue was diluted with water (100 mL) and extracted with ethyl acetate (3×100 mL). The combined organic phases were concentrated in vacuo. The residue was purified by flash chromatography (ethyl acetate/petroleum ether=0% to 100%) to yield 3-cyano-4-methyl-N-(4-methyl-3-((3-(9-(tetrahydro-2H-pyran-2-yl)-9H-purin-6-yl)pyridin-2-yl)amino)phenyl)benzamide (60 mg, 0.11 mmol, 37%) as a white solid. MS (ESI) m/z 545 [M+H]⁺

Step 3. 3-Cyano-4-methyl-N-(4-methyl-3-((3-(9-(tetrahydro-2H-pyran-2-yl)-9H-purin-6-yl)pyridin-2-yl)amino)phenyl)benzamide (54 mg, 0.1 mmol) was dissolved in trifluoroacetic acid/dichloromethane (1 mL/4 mL) and stirred at room temperature for 4 h. The mixture was concentrated in vacuo. The residue was diluted with saturated sodium bicarbonate solution (100 mL) and extracted with ethyl acetate (3×100 mL). The combined organic phases were concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to yield N-(3-((3-(9H-purin-6-yl)pyridin-2-yl)amino)-4-methylphenyl)-3-cyano-4-methylbenzamide (10 mg, 0.022 mmol, 22%) as a yellow solid. ¹H NMR (400 MHZ, DMSO-d₆) δ 12.86 (s, 1H), 10.28 (s, 1H), 9.97 (d, J=6.7 Hz, 1H), 8.79 (s, 1H), 8.72 (s, 1H), 8.40 (s, 1H), 8.35-8.21 (m, 2H), 8.18 (d, J=7.1 Hz, 1H), 7.63 (d, J=8.0 Hz, 1H), 7.45 (d, J=7.6 Hz, 1H), 7.21 (d, J=8.3 Hz, 1H), 6.97 (dd, J=7.6, 4.9 Hz, 1H), 2.57 (s, 3H), 2.46 (s, 3H). MS (ESI) m/z 461 [M+H]⁺

### Example 47. Synthesis of N-(3-((3-(9H-purin-6-yl)pyridin-2-yl)amino)-4-methylphenyl)-3-chloro-4-methylbenzamide (Compound I-44)

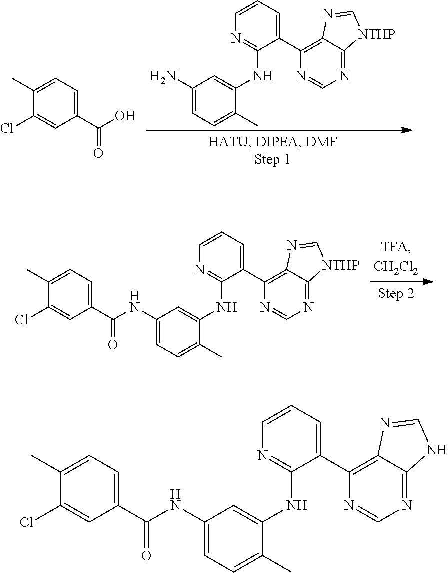

Step 1. To a mixture of 3-chloro-4-methylbenzoic acid (30 mg, 0.18 mmol) in N,N-dimethylformamide (3 mL) was added N,N-diisopropylethylamine (47 mg, 0.36 mmol) and 1-[bis(dimethylamino)methylene]-1H-1,2,3-triazolo[4,5-b]pyridinium 3-oxid hexafluorophosphate (82 mg, 0.22 mmol). The mixture was stirred at room temperature for 0.5 h. Then 6-methyl-N1-(3-(9-(tetrahydro-2H-pyran-2-yl)-9H-purin-6-yl)pyridin-2-yl)benzene-1,3-diamine (72 mg, 0.18 mmol) was added, and the mixture was stirred at room temperature for 2 h. The reaction mixture was poured into water (10 mL), and then diluted with ethyl acetate (20 mL). The organic layer was washed with water (20 mL×3) and brine (20 mL), dried over sodium sulfate, filtered and concentrated. The residue was purified by flash chromatography (petroleum ether/ethyl acetate=1/1) to give 3-chloro-4-methyl-N-(4-methyl-3-((3-(9-(tetrahydro-2H-pyran-2-yl)-9H-purin-6-yl)pyridin-2-yl)amino)phenyl)benzamide (80 mg, 0.14 mmol, 80%) as a yellow solid. MS (ESI) m/z 553.9 [M+H]⁺

Step 2. A mixture of 3-chloro-4-methyl-N-(4-methyl-3-((3-(9-(tetrahydro-2H-pyran-2-yl)-9H-purin-6-yl)pyridin-2-yl)amino)phenyl)benzamide (80 mg, 0.14 mmol) in trifluoroacetic acid/dichloromethane (3 mL/3 mL) was stirred at room temperature for 2 h. On completion, the reaction mixture was concentrated under reduced pressure to give a residue which was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-3-chloro-4-methylbenzamide (4.3 mg, 0.009 mmol, 6%) as a yellow solid. ¹H NMR (400 MHZ, DMSO-d₆) δ 12.32 (s, 1H), 10.23 (s, 1H), 9.82 (s, 1H), 9.09 (s, 1H), 8.72 (s, 1H), 8.65 (d, J=1.6 Hz, 1H), 8.36 (dd, J=4.8, 2.0 Hz, 1H), 8.05 (d, J=1.6 Hz, 1H), 7.89-7.86 (m, 1H), 7.52 (d, J=8.0 Hz, 1H), 7.46 (s, 1H), 7.23 (d, J=8.4 Hz, 1H), 7.04 (dd, J=8.0, 4.8 Hz, 1H), 2.43 (d, J=7.2 Hz, 6H). MS (ESI) m/z 469.9 [M+H]⁺

### Example 48. Synthesis of N-(3-((3-(9H-purin-6-yl)pyridin-2-yl)amino)-4-methylphenyl)-4-chloro-3,5-dicyanobenzamide (Compound I-45)

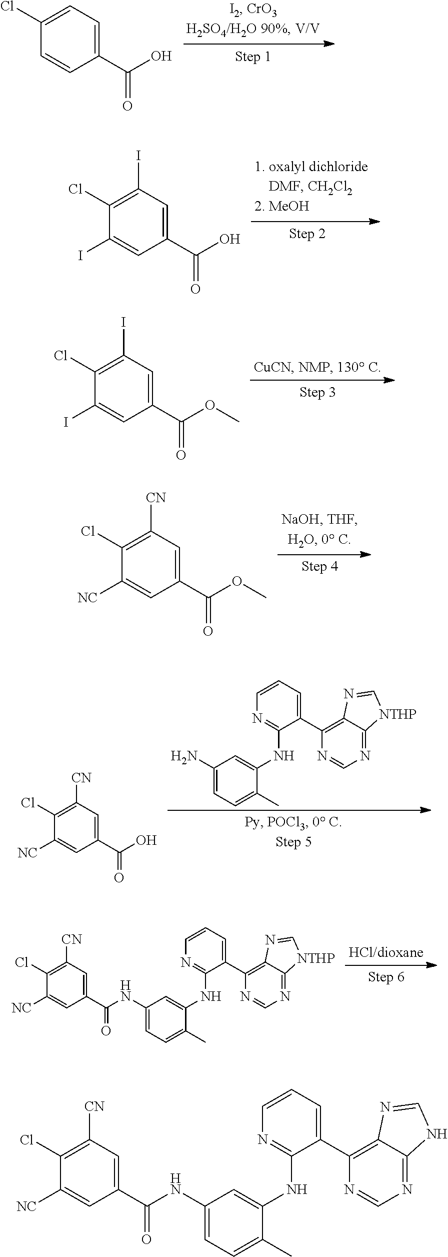

Step 1. To a solution of iodine (4.54 g, 17.9 mmol) in sulfuric acid (90% in water by volume, 100 mL) was added chromium(VI) oxide (1.79 g, 17.9 mmol) at 25° C., and stirred at 25° C. for 30 minutes. To the solution was added 4-chlorobenzoic acid (2.16 g, 13.77 mmol). The mixture was stirred at 25° C. for 24 h and then poured into ice/water (50 mL) and filtered. The solid was washed with cool water (50 mL) and then dried under vacuum at 50° C. to afford 4-chloro-3,5-diiodobenzoic acid (3.1 g, 7.59 mmol, 55%) as a yellow solid. ¹H NMR (400 MHZ, DMSO-d₆) δ 8.35 (s, 2H).

Step 2. To a solution of 4-chloro-3,5-diiodobenzoic acid (3.1 g, 7.59 mmol) in dichloromethane (100 mL) was added oxalyl dichloride (1.25 mg, 9.87 mmol) and N,N-dimethylformamide (0.2 mL) slowly at 0° C. under argon. The mixture was stirred at 25° C. for 3 h. Then the solvent was removed to afford the acid chloride, and methanol (20 mL) was added dropwise. After the addition, the reaction was stirred at 0° C. for 10 min, then 25° C. for 1 h. The mixture was concentrated in vacuo. The residue was purified by flash chromatography (ethyl acetate/petroleum ether from 5% to 26%) to give methyl 4-chloro-3,5-diiodobenzoate (2.8 g, 6.63 mmol, 87%) as a yellow solid. ¹H NMR (400 MHZ, CDCl₃) δ 8.56-8.37 (m, 2H), 3.93 (d, J=4.0 Hz, 3H).

Step 3. To a suspension of methyl 4-chloro-3,5-diiodobenzoate (1.1 g, 2.6 mmol) in 1-methyl-2-pyrrolidinone (12 mL) was added copper(I) cyanide (559 mg, 6.24 mmol), followed by exchange of argon three times. The reaction was stirred at 130° C. for 16 h. The mixture was diluted with ethyl acetate (150 mL) and washed with water (150 mL). The organic phase was washed with brine (40 mL×3), dried over sodium sulfate, filtered and concentrated. The residue was purified by flash chromatography (ethyl acetate/petroleum ether=23/100) to afford methyl 4-chloro-3,5-dicyanobenzoate (350 mg, 1.59 mmol, 61%) as a yellow solid. ¹H NMR (400 MHZ, CDCl₃) δ8.53 (d, J=3.8 Hz, 2H), 4.01 (s, 3H).

Step 4. To a solution of methyl 4-chloro-3,5-dicyanobenzoate (150 mg, 0.68 mmol) in tetrahydrofuran (5 mL) was added sodium hydroxide (52 mg, 1.36 mmol) and water (5 mL). The mixture was stirred at 0° C. for 1 h. The solvent was evaporated under reduced pressure, and the residue was diluted with water (10 mL). The mixture was adjusted to pH 3 by adding 1 N hydrochloric acid slowly. The resulting white precipitate was collected by filtration and washed with water (5 mL) to afford 4-chloro-3,5-dicyanobenzoic acid (104 mg, 0.5 mmol, 74%) as a yellow solid. ¹H NMR (400 MHZ, DMSO-d₆): δ14.06 (s, 1H), 8.69 (s, 2H).

Step 5. To an ice-cooled solution of 6-methyl-N1-(3-(9-(tetrahydro-2H-pyran-2-yl)-9H-purin-6-yl)pyridin-2-yl)benzene-1,3-diamine (123 mg, 0.31 mmol) and 4-chloro-3,5-dicyanobenzoic acid (84 mg, 0.4 mmol) in dry pyridine (5 mL) was added dropwise phosphorus oxychloride (143 mg, 0.93 mmol). The mixture was stirred at 0° C. for 1 h. The mixture was diluted with ethyl acetate (50 mL) and washed with saturated aqueous sodium bicarbonate solution (25 mL) and brine (25 mL). The organic layer was dried with sodium sulfate, filtered and concentrated. The residue was purified by flash chromatography (ethyl acetate/petroleum ether from 10% to 50%) to give 4-chloro-3,5-dicyano-N-(4-methyl-3-(3-(9-(tetrahydro-2H-pyran-2-yl)-9H-purin-6-yl)pyridin-2-ylamino)phenyl)benzamide (74 mg, 0.125 mmol, 40%) as a yellow solid. MS (ESI) m/z 589.8 [M+H]⁺

Step 6. To a solution of 4-chloro-3,5-dicyano-N-(4-methyl-3-(3-(9-(tetrahydro-2H-pyran-2-yl)-9H-purin-6-yl)pyridin-2-ylamino)phenyl)benzamide (74 mg, 0.125 mmol) in dichloromethane (5 mL) was added hydrogen chloride solution (4 M in dioxane, 10 mL) at 25° C., and stirred at 25° C. for 10 h. After completed, the reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-4-chloro-3,5-dicyanobenzamide (5.4 mg, 0.011 mmol, 9%) as a yellow solid. ¹H NMR (400 MHZ, DMSO-d₆) δ 12.38 (s, 1H), 10.53 (s, 1H), 9.83 (s, 1H), 9.09 (s, 1H), 8.82 (s, 2H), 8.74-8.67 (m, 2H), 8.37 (dd, J=4.7, 1.9 Hz, 1H), 7.52-7.44 (m, 1H), 7.30-7.22 (m, 1H), 7.08-7.01 (m, 1H), 2.58-2.53 (m, 3H). MS (ESI) m/z 505.9 [M+H]⁺

### Example 49. Synthesis of N-(3-((3-(9H-purin-6-yl)pyridin-2-yl)amino)-4-methylphenyl)-4-cyano-5-(trifluoromethyl)picolinamide (Compound I-46)

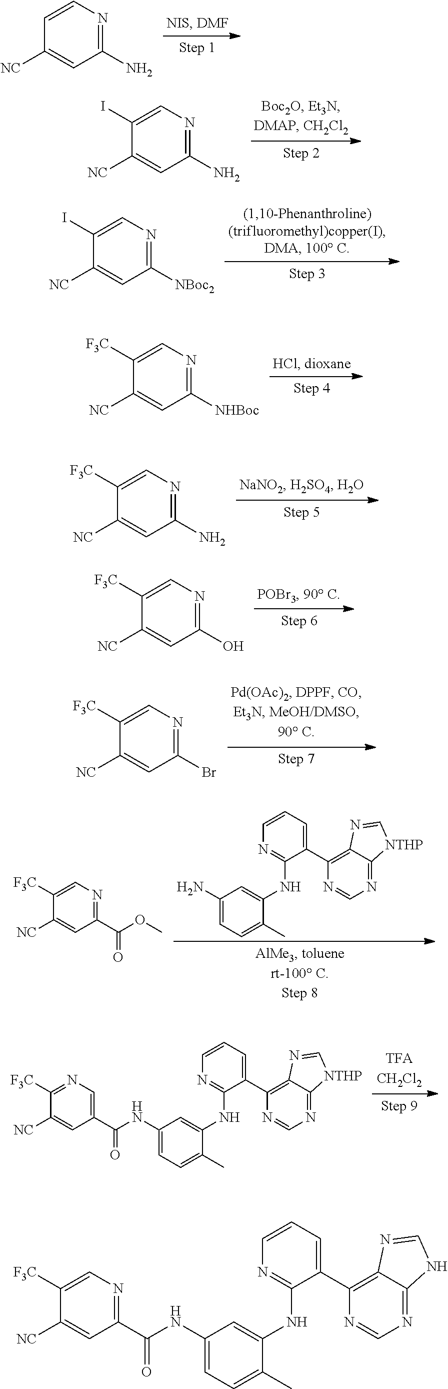

Step 1. To a stirred solution of 2-aminoisonicotinonitrile (5.6 g, 47.0 mmol) in dimethylformamide (50 mL) was added N-iodosuccinimide (12.6 g, 56.4 mmol) slowly. The reaction mixture was stirred at room temperature for 16 h. Then water (20 mL) was added to precipitate a brown solid. The precipitate was collected by filtration and washed with water (10 mL) to give 2-amino-5-iodoisonicotinonitrile0 (10.0 g, 40.8 mmol, 87%) as a brown solid. MS (ESI) m/z 245.8 [M+H]⁺

Step 2. A solution of 2-amino-5-iodoisonicotinonitrile (1.9 g, 7.75 mmol), di-tert-butyldicarbonate (5.23 g, 23.3 mmol), triethanolamine (2.35 g, 23.3 mmol) and 4-dimethylaminopyridine (94 mg, 0.775 mmol) in dichloromethane (30 mL) was stirred at room temperature for 16 h. Then the mixture reaction was concentrated and purified by flash chromatography (petroleum ether/ethyl acetate=1/1) to give di-tert-butyl(4-cyano-5-iodopyridin-2-yl)iminodicarbonate (3.45 g, 7.75 mmol, 100%) as a yellow solid. MS (ESI) m/z 467.8 [M+H]⁺

Step 3. A solution of di-tert-butyl(4-cyano-5-iodopyridin-2-yl)iminodicarbonate (3.0 g, 6.74 mmol) and (1,10-phenanthroline)(trifluoromethyl)copper(I) (3.15 g, 10.11 mmol) in dimethylacetamide (30 mL) was stirred at 100° C. for 16 h. Then water (50 mL) was added, the mixture was extracted with ethyl acetate (80 mL) and washed with brine (30 mL). The organic phase was dried over sodium sulfate, filtered and concentrated. The residue was purified by flash chromatography (petroleum ether/ethyl acetate=2/1) to give tert-butyl (4-cyano-5-(trifluoromethyl)pyridine-2-yl)carbamate (1.3 g, 4.52 mmol, 67%) as a yellow solid. MS (ESI) m/z 232.1 [M+H]⁺

Step 4. A solution of tert-butyl (4-cyano-5-(trifluoromethyl)pyridine-2-yl)carbamate (1.3 g, 4.52 mmol) in hydrogen chloride solution (4 M in dioxane, 15 mL) was stirred at room temperature for 16 h. The reaction was concentrated and triturated with diethyl ether (20 mL) to give 2-amino-5-(trifluoromethyl)isonicotinonitrile (1.2 g, 6.41 mmol) as a white solid. MS (ESI) m/z 188.1 [M+H]⁺

Step 5. Sodium nitrite (1.47 g, 21.4 mmol) in water (10 mL) was added dropwise to a solution of 2-amino-5-(trifluoromethyl)isonicotinonitrile (1.0 g, 5.34 mmol) in water (8 mL) and sulfuric acid (3 mL) at 0° C. The reaction mixture was stirred at room temperature for 16 h. The mixture was filtered, the filtrate was adjusted to pH-8 with 2 N sodium hydroxide, then extracted with ethyl acetate (80 mL). The organic layer was washed with brine (30 mL) and dried over sodium sulfate, filtered and concentrated to give 2-hydroxy-5-(trifluoromethyl)isonicotinonitrile (730 mg, 3.88 mmol, 73%) as a yellow solid. MS (ESI) m/z 189.1 [M+H]⁺

Step 6. A solution of 2-hydroxy-5-(trifluoromethyl)isonicotinonitrile (420 mg, 2.23 mmol) in phosphorus(V) tribromide oxide (5 mL) was stirred at 90° C. for 1 h. Then ice water (10 mL) was added to the reaction mixture slowly and extracted with ethyl acetate (50 mL). The organic layer was washed with brine (20 mL) and dried over sodium sulfate, filtered, and concentrated. The residue was purified by flash chromatography (petroleum ether/ethyl acetate=4/1) to give 2-bromo-5-(trifluoromethyl)isonicotinonitrile (420 mg, 1.67 mmol, 75%) as a brown solid. ¹H NMR (500 MHZ, DMSO-d₆) δ 9.07 (s, 1H), 8.64 (s, 1H).

Step 7. A solution of 2-bromo-5-(trifluoromethyl)isonicotinonitrile (600 mg, 2.39 mmol), 1, l′-bis(diphenylphosphino)ferrocene (264 mg, 0.48 mmol), palladium (II) acetate (54 mg, 0.24 mmol) and triethylamine (483 mg, 4.78 mmol) in methanol/dimethylsulfoxide (2/3, 8 mL) was stirred at 90° C. for 16 h under carbon monoxide. The reaction mixture was diluted with ethyl acetate (80 mL) and washed with water (20 mL×3), then brine (30 mL). The organic phase was dried over sodium sulfate, filtered, and concentrated. The residue was purified by flash chromatography (petroleum ether/ethyl acetate=1/1) to give methyl 4-cyano-5-(trifluoromethyl)picolinate (100 mg, 0.43 mmol, 18%) as a yellow solid. MS (ESI) m/z 230.9 [M+H]⁺

Step 8. To a solution of 6-methyl-N1-(3-(9-(tetrahydro-2H-pyran-2-yl)-9H-purin-6-yl)pyridin-2-yl)benzene-1,3-diamine (35 mg, 0.087 mmol) in toluene (2 mL) was added trimethylaluminium (0.3 mL, 0.26 mmol) slowly at 0° C. under argon. The mixture was stirred at 25° C. for 2 h. Then methyl 4-cyano-5-(trifluoromethyl)picolinate (20 mg, 0.087 mmol) in toluene (2.0 mL) was added to the mixture, the resulting mixture was heated to 100° C. and stirred for 3 h. After completed, the mixture was cooled to 25° C., ice was added slowly, then water (5.0 mL) and ethyl acetate (10 mL) were added. The mixture was filtered and washed with ethyl acetate to give 4-cyano-N-(4-methyl-3-((3-(9-(tetrahydro-2H-pyran-2-yl)-9H-purin-6-yl)pyridin-2-yl)amino)-phenyl)-5-(trifluoromethyl)picolinamide (50 mg, 0.083 mmol, 96%) as a yellow solid. MS (ESI) m/z 599.8 [M+H]⁺

Step 9. A solution of 4-cyano-N-(4-methyl-3-((3-(9-(tetrahydro-2H-pyran-2-yl)-9H-purin-6-yl)pyridin-2-yl)amino)phenyl)-5-(trifluoromethyl)picolinamide (50 mg, 0.083 mmol) in trifluoroacetic acid/dichloromethane (2 mL/2 mL) was stirred at room temperature for 1 h. The mixture was concentrated, the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-4-cyano-5-(trifluoromethyl)picolin amide (3.7 mg, 0.007 mmol, 9%) as a yellow solid. ¹H NMR (400 MHZ, DMSO-d₆) δ 13.88 (s, 1H), 12.34 (s, 1H), 10.77 (s, 1H), 9.79 (d, J=5.6 Hz, 1H), 9.34 (s, 1H), 9.08 (s, 1H), 8.83 (d, J=2.0 Hz, 1H), 8.75 (s, 1H), 8.70 (s, 1H), 8.36 (dd, J=4.4 Hz, 1.6 Hz, 1H), 7.53 (dd, J=8.0 Hz, 2.0 Hz, 1H), 7.27 (d, J=8.4 Hz, 1H), 7.05 (dd, J=8.0 Hz, 4.8 Hz, 1H), 2.45 (s, 3H). MS (ESI) m/z 515.8 [M+H]⁺

### Example 50. Synthesis of N-(3-((3-(9H-purin-6-yl)pyridin-2-yl)amino)-4-methylphenyl)-2-(3-(trifluoromethyl)cyclohexyl)acetamide (Compound I-47)

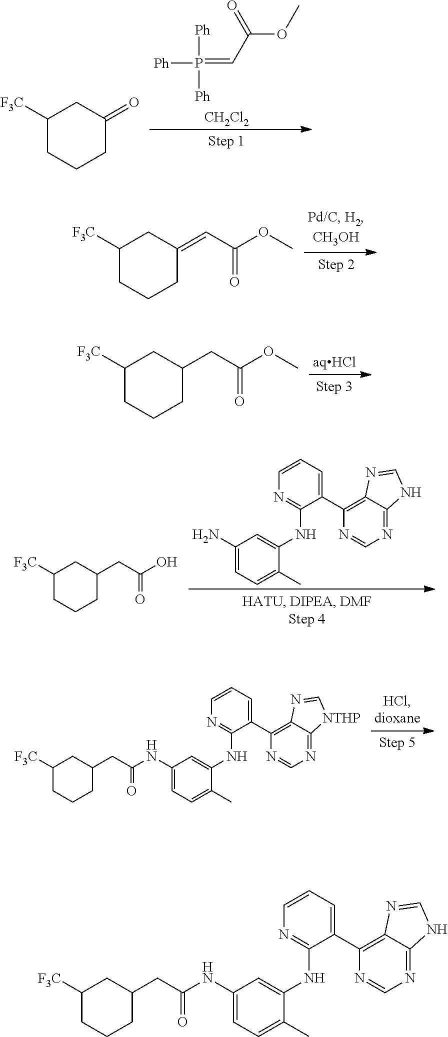

Step 1. A mixture of 3-(trifluoromethyl)cyclohexan-1-one (800 mg, 4.819 mmol) and methyl 2-(triphenyl-15-phosphaneylidene)acetate (2487 mg, 7.229 mmol) in dichloromethane (25 mL) was stirred at 25° C. for 16 h. The mixture was dilute with ethyl acetate and water was added. The mixture was extracted with ethyl acetate (30 mL×3). The organic layer was concentrated. The crude residue was purified by silica gel column chromatography (petroleum ether/ethyl acetate=50/1 to 5/1) to give methyl (E)-2-(3-(trifluoromethyl)cyclohexylidene)acetate (800 mg, 3.6 mmol, 74%) as a yellow oil. MS (ESI) m/z 223.1 [M+H]⁺

Step 2. A solution of methyl (E)-2-(3-(trifluoromethyl)cyclohexylidene)acetate (220 m, 0.991 mmol) and palladium (10% on carbon, 30 mg) in methanol (8 mL) was stirred at 25° C. under hydrogen for 1 h. The mixture was filtered and the filtrate was concentrated to give methyl 2-(3-(trifluoromethyl)cyclohexyl)acetate (180 mg, 0.8 mmol, 82%) as a yellow oil. MS (ESI) m/z 225.1 [M+H]⁺

Step 3. A solution of methyl 2-(3-(trifluoromethyl)cyclohexyl)acetate (180 mg, 0.8 mmol) and hydrogen chloride (2 mL) in tetrahydrofuran/water (8 mL) was stirred at 25° C. for 2 h. The mixture was concentrated to give 2-(3-(trifluoromethyl)cyclohexyl)acetic acid (90 mg, 0.428 mmol, 51%) as a yellow oil. MS (ESI) m/z 211.1 [M+H]⁺

Step 4. To a mixture of 2-(3-(trifluoromethyl)cyclohexyl)acetic acid (60 mg, 0.286 mmol) in N,N-dimethyl formamide (6 mL) was added 6-methyl-N1-(3-(9-(tetrahydro-2H-pyran-2-yl)-9H-purin-6-yl)pyridin-2-yl)benzene-1,3-diamine (137 mg, 0.343 mmol), 1-[bis(dimethylamino)-methylene]-1H-1,2,3-triazolo[4,5-b]pyridinium 3-oxid hexafluorophosphate (163 mg, 0.429 mmol), and N,N-diisopropylethylamine (110 mg, 0.858 mmol) at 25° C. The mixture was stirred at room temperature for 2 h. On completion, the reaction mixture was quenched by addition water (50 mL) and extracted with ethyl acetate (100 mL×3). The combined organic layers were washed with water (50 mL×2), dried over sodium sulfate, filtered and concentrated under reduced pressure to give a residue, which was purified by column chromatography (petroleum ether/ethyl acetate=10/1 to 1/1) to give N-(4-methyl-3-((3-(9-(tetrahydro-2H-pyran-2-yl)-9H-purin-6-yl)pyridin-2-yl)amino)phenyl)-2-(3-(trifluoromethyl)cyclohexyl)acetamide (90 mg, 0.152 mmol, 53%) as a yellow solid. MS (ESI) m/z 594.3 [M+H]⁺

Step 5. A solution of N-(4-methyl-3-((3-(9-(tetrahydro-2H-pyran-2-yl)-9H-purin-6-yl)pyridin-2-yl)amino)phenyl)-2-(3-(trifluoromethyl)cyclohexyl)acetamide (90 mg, 0.152 mmol) in trifluoroacetic acid (5 mL) was stirred at 50° C. for 1 h. The mixture was concentrated. The crude residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-2-(3-(trifluoromethyl)-cyclohexyl)acetamide (24.1 mg, 0.047 mmol, 31%) as a yellow solid. ¹H NMR (400 MHZ, DMSO-d₆) δ 12.24 (s, 1H), 9.80 (s, 1H), 8.70 (s, 1H), 88.42-8.43 (m, 1H), 8.33-8.31 (m, 1H), 7.37-7.39 (d, J=8.0 Hz, 1H), 7.14-7.16 (d, J=8.4 Hz, 1H), 7.01-7.04 (m, 1H), 2.39 (s, 3H), 2.31-2.34 (m, 2H), 2.24-2.27 (m, 1H), 1.77-1.89 (m, 4H), 1.51-1.57 (m, 2H), 1.16-1.35 (m, 3H), 0.93-0.99 (m, 1H). MS (ESI) m/z 510.1 [M+H]⁺

### Example 51. Synthesis of N-(3-(3-(9H-purin-6-yl)pyridin-2-ylamino)-4-methylphenyl)-5-cyano-6-methylnicotinamide (Compound I-48)

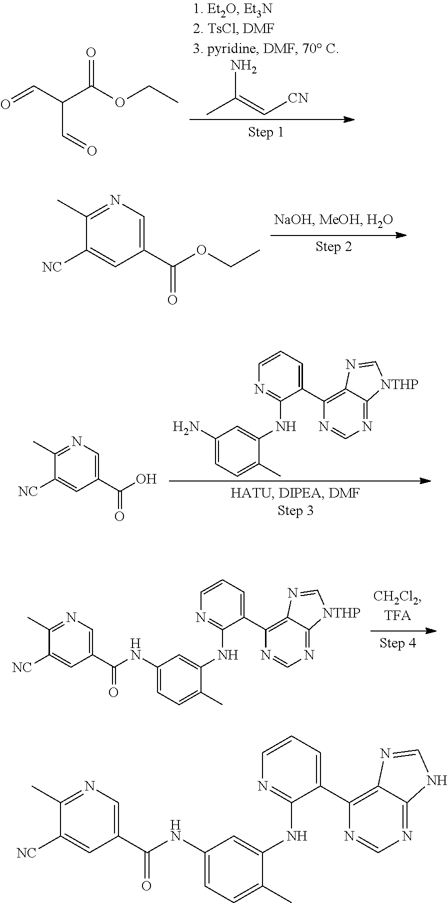

Step 1. To a solution of ethyl 2-formyl-3-oxopropanoate (1000 mg, 7 mmol) in anhydrous ether (3 mL) was added triethylamine (800 mg, 7.7. mmol) at −10° C. and the mixture is stirred for 1 h at room temperature. Ether was then removed in vacuo and to the residue was added a solution of tosyl chloride (1440 mg, 7.7 mmol) in dimethylformamide (4 mL) with stirring at −20° C. The mixture was allowed to gradually warm to room temperature and stirring was continued for an additional 3 h at room temperature. A solution of (Z)-3-aminobut-2-enenitrile (600 mg, 7 mmol) and pyridine (2220 mg, 28 mmol) in dimethylformamide (4 mL) was added and the mixture was heated at 70° C. for 10 h. Most of the solvent was removed under vacuum and the residue was extracted with chloroform (20 mL×3). The organic extract was washed with saturated sodium hydrogen carbonate solution (10 mL) and with brine (30 mL), and was dried over sodium sulfate. The solvent was evaporated and the residue was purified by silica gel column chromatography (ethyl acetate/hexane=1/3) to afford ethyl 5-cyano-6-methylnicotinate (540 mg, 2.8 mmol, 41%). MS (ESI) m/z 191.1 [M+H]⁺

Step 2. To a solution of ethyl 5-cyano-6-methylnicotinate (540 mg, 2.8 mmol) in methanol (3 mL) and water (1 mL) was added sodium hydroxide (227 mg, 5.7 mmol). The mixture was stirred at room temperature for 30 min. The reaction mixture was extracted with ethyl acetate (20 mL×2) and washed with water (20 mL×2). The organic layer was evaporated to give 5-cyano-6-methylnicotinic acid (360 mg, 2.22 mmol, 78%) as a white solid. MS (ESI) m/z 163.1 [M+H]⁺

Step 3. A solution of 5-cyano-6-methylnicotinic acid (32 mg, 0.2 mmol), 6-methyl-N1-(3-(9-(tetrahydro-2H-pyran-2-yl)-9H-purin-6-yl)pyridin-2-yl)benzene-1,3-diamine (80 mg, 0.2 mmol), 1-[bis(dimethylamino) methylene]-1H-1,2,3-triazolo[4,5-b]pyridinium 3-oxid hexafluorophosphate (114 mg), N,N-diisopropylethylamine (77 mg) in N,N-dimethylformamide (3 mL) was stirred at room temperature for 3 h. The mixture was extracted with ethyl acetate (50 mL×2) and washed with water (30 mL×2), dried and concentrated to give 5-cyano-6-methyl-N-(4-methyl-3-((3-(9-(tetrahydro-2H-pyran-2-yl)-9H-purin-6-yl)pyridin-2-yl)amino)phenyl)-nicotinamide (100 mg, 0.18 mmol, 93%). MS (ESI) m/z 546.1 [M+H]⁺

Step 4. A solution of 5-cyano-6-methyl-N-(4-methyl-3-((3-(9-(tetrahydro-2H-pyran-2-yl)-9H-purin-6-yl)pyridin-2-yl)amino)phenyl)nicotinamide (100 mg, 0.18 mmol), trifluoroacetic acid (46 mg, 0.4 mmol) in dichloromethane (3 mL) was stirred at room temperature for 2 h. The mixture was extracted with ethyl acetate (50 mL×2) and washed with water (30 mL×2). The organic layer was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-5-cyano-6-methylnicotinamide (13.5 mg, 0.029 mmol, 16%) as a yellow solid. ¹H NMR (400 MHZ, DMSO-d₆) δ10.43 (s, 1H), 9.22 (s, 1H), 8.88 (s, 1H), 8.25 (dd, J=2.0, 14.8 Hz, 2H), 7.95 (dd, J=2.4, 8.4 Hz, 1H), 7.83 (d, J=8.4 Hz, 2H), 7.76 (s, 2H), 7.36 (d, J=8.8 Hz, 1H), 2.95 (d, J=4.4 Hz, 3H), 2.28 (s, 3H). MS (ESI) m/z 462.1 [M+H]⁺

### Example 52. Synthesis of N-(3-(3-(9H-purin-6-yl)pyridin-2-ylamino)-4-methylphenyl)-3,4-dichlorobenzamide (Compound I-49)

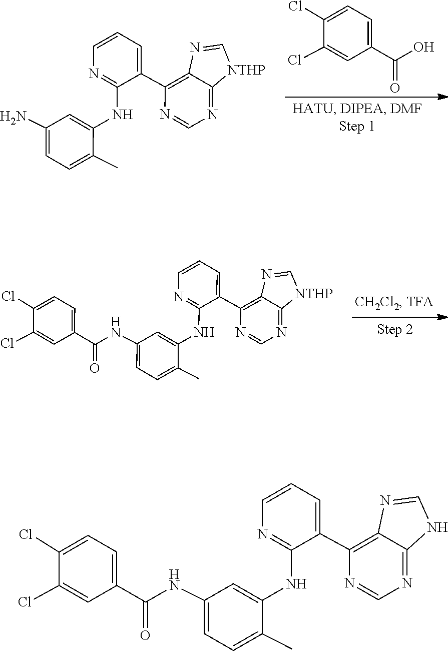

Step 1. A solution of 3,4-dichlorobenzoic acid (38 mg, 0.2 mmol), 6-methyl-N1-(3-(9-(tetrahydro-2H-pyran-2-yl)-9H-purin-6-yl)pyridin-2-yl)benzene-1,3-diamine (80 mg, 0.2 mmol), 1-[bis(dimethylamino)methylene]-1H-1,2,3-triazolo[4,5-b]pyridinium 3-oxid hexafluorophosphate (114 mg, 0.3 mmol), N,N-diisopropylethylamine (77 mg, 0.6 mmol) in N,N-dimethylformamide (3 mL) was stirred at room temperature for 2 h. The reaction mixture was extracted with ethyl acetate (50 mL×2) and washed with water (30 mL×2) to give 3,4-dichloro-N-(4-methyl-3-((3-(9-(tetrahydro-2H-pyran-2-yl)-9H-purin-6-yl)pyridin-2-yl)amino)phenyl)-benzamide (100 mg, 0.174 mmol, 88%). MS (ESI) m/z 574.1 [M+H]⁺

Step 2. A solution of 3,4-dichloro-N-(4-methyl-3-((3-(9-(tetrahydro-2H-pyran-2-yl)-9H-purin-6-yl)pyridin-2-yl)amino)phenyl)benzamide (90 mg, 0.16 mmol) and trifluoroacetic acid (56 mg, 0.5 mmol) in dichloromethane (3 mL) was stirred at room temperature for 1 h. The reaction mixture was extracted with ethyl acetate (20 mL×2) and washed with water (20 mL×2). The organic layer was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methyl phenyl)-3,4-dichlorobenzamide (9.4 mg, 0.019 mmol, 12%) as a yellow solid. ¹H NMR (400 MHz, DMSO-d₆) δ 13.84 (br s, 1H), 12.33 (bs, 1H), 10.35 (s, 1H), 9.82 (bs, 1H), 9.08 (s, 1H), 8.72 (s, 1H), 8.67 (s, 1H), 8.36 (dd, J=1.6, 4.4 Hz, 1H), 8.24 (d, J=2.0 Hz, 1H), 7.96 (dd, J=4.0, 8.4 Hz, 1H), 7.81 (d, J=8.1 Hz, 1H), 7.46 (d, J=9.6 Hz, 1H), 7.23 (d, J=8.0 Hz, 1H), 7.04 (dd, J=4.8, 8.4 Hz, 1H), 2.44 (s, 3H). MS (ESI) m/z 490.0 [M+H]⁺

### Example 53. Synthesis of N-(3-(3-(9H-purin-6-yl)pyridin-2-ylamino)-4-fluorophenyl)-4-chloro-3-cyanobenzamide (Compound I-50)

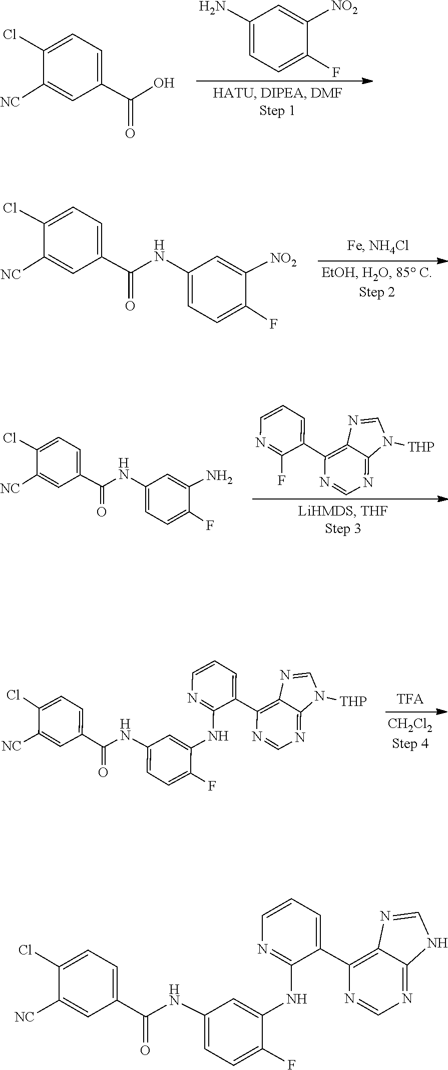

Step 1. A mixture of 4-chloro-3-cyanobenzoic acid (364 mg, 2 mmol), 4-fluoro-3-nitroaniline (312 mg, 2 mmol), 1-[bis(dimethylamino)methylene]-1H-1,2,3-triazolo[4,5-b]pyridinium 3-oxid hexafluorophosphate (1140 mg, 3 mmol) and N,N-diisopropylethylamine (516 mg, 4 mmol) in N,N-dimethylformamide (4 mL) was stirred at room temperature for 4 h. The mixture was concentrated in vacuo. The residue was diluted with water (100 mL) and extracted with ethyl acetate (3×100 mL). The combined organic phases were concentrated in vacuo. The residue was purified by silica gel flash chromatography (ethyl acetate/petroleum ether=0% to 33%) to afford 4-chloro-3-cyano-N-(4-fluoro-3-nitrophenyl)benzamide (400 mg, 1.25 mmol, 63%) as a white solid. MS (ESI) m/z 320 [M+H]⁺

Step 2. 4-chloro-3-cyano-N-(4-fluoro-3-nitrophenyl)benzamide (319 mg, 1 mmol), iron powder (280 mg, 5 mmol) and ammonium chloride (265 mg, 53 mmol) were suspended in ethanol/water (8 mL/2 mL) and stirred at 85° C. for 2 h. The mixture was concentrated in vacuo. The residue was diluted with water (100 mL) and extracted with ethyl acetate (3×100 mL). The combined organic phases were concentrated in vacuo. The residue was purified by silica gel flash chromatography (ethyl acetate/petroleum ether=0% to 67%) to afford N-(3-amino-4-fluorophenyl)-4-chloro-3-cyanobenzamide (200 mg, 0.69 mmol, 69%) as a white solid. MS (ESI) m/z 290 [M+H]⁺

Step 3. A mixture of N-(3-amino-4-fluorophenyl)-4-chloro-3-cyanobenzamide (193 mg, 0.67 mmol), 6-(2-fluoropyridin-3-yl)-9-(tetrahydro-2H-pyran-2-yl)-9H-purine (200 mg, 0.67 mmol) and lithium hexamethyldisilazide solution (2.09 mL, 3.35 mmol) in tetrahydrofuran (10 mL) was stirred at room temperature for 16 h. The mixture was concentrated in vacuo. The residue was diluted with water (100 mL) and extracted with ethyl acetate (3×100 mL). The combined organic phases were concentrated in vacuo. The residue was purified by silica gel flash chromatography (ethyl acetate/petroleum ether=0% to 33%) to afford 4-chloro-3-cyano-N-(4-fluoro-3-((3-(9-(tetrahydro-2H-pyran-2-yl)-9H-purin-6-yl)pyridin-2-yl)amino)phenyl)benzamide (100 mg, 0.176 mmol, 26%) as a white solid. MS (ESI) m/z 569 [M+H]⁺

Step 4. A mixture of 4-chloro-3-cyano-N-(4-fluoro-3-((3-(9-(tetrahydro-2H-pyran-2-yl)-9H-purin-6-yl)pyridin-2-yl)amino)phenyl)benzamide (113 mg, 0.2 mmol) in trifluoroacetic acid/dichloromethane (1 mL/5 mL) was stirred at room temperature for 4 h. The mixture was concentrated in vacuo. The residue was diluted with saturated sodium bicarbonate aqueous solution (100 mL) and extracted with ethyl acetate (3×100 mL). The combined organic phases were concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-fluorophenyl)-4-chloro-3-cyanobenzamide (20 mg, 0.041 mmol, 21%) as a yellow solid. ¹H NMR (400 MHZ, DMSO-d₆) & 13.81 (s, 1H), 12.88 (s, 1H), 10.53 (s, 1H), 9.84 (s, 1H), 9.06-8.94 (m, 2H), 8.72 (s, 1H), 8.58 (d, J=2.1 Hz, 1H), 8.43 (dd, J=4.7, 1.8 Hz, 1H), 8.28 (dd, J=8.5, 2.1 Hz, 1H), 7.94 (d, J=8.6 Hz, 1H), 7.50 (d, J=2.9 Hz, 1H), 7.31 (dd, J=11.1, 8.9 Hz, 1H), 7.12 (dd, J=7.9, 4.7 Hz, 1H). MS (ESI) m/z 485 [M+H]⁺

### Example 54. Synthesis of N-(3-(3-(9H-purin-6-yl)pyridin-2-ylamino)-4-methylphenyl)-6-cyano-5-methylpicolinamide (Compound I-51)

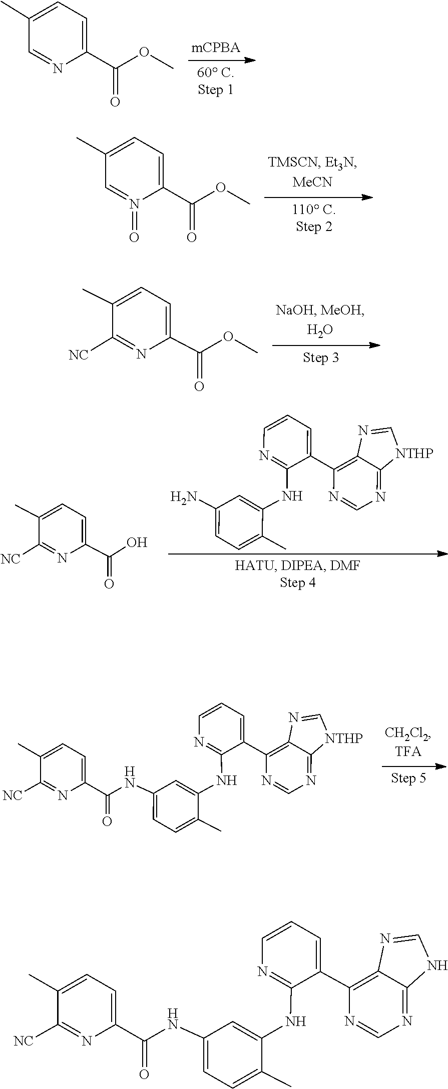

Step 1. To a solution of methyl 5-methylpicolinate (1510 mg, 10 mmol) in dichloromethane (8 mL) was added 3-chloroperbenzoic acid (2420 mg, 14 mmol). The mixture was stirred at 60° C. for 6 h. The reaction mixture was extracted with ethyl acetate (50 mL×2) and washed with water (30 mL×2). The organic layer was purified by silica gel column chromatography (methanol/dichloromethane=4/96) to give 2-(methoxycarbonyl)-5-methylpyridine 1-oxide (600 mg, 2.62 mmol, 36%) as a white solid. MS (ESI) m/z 230.1 [M+H]⁺

Step 2. To a solution of 2-(methoxycarbonyl)-5-methylpyridine 1-oxide (270 mg, 1.6 mmol) in acetonitrile (4 mL) was added trimethylsilyl cyanide (400 mg, 4 mmol) and triethylamine (243 mg, 2.4 mmol). The mixture was stirred at 110° C. for 12 h. The mixture was extracted with ethyl acetate (10 mL×2) and washed with water (20 mL×2). The organic layer was concentrated and purified by prep-TLC (Silica, UV 254 nm, petroleum ether/ethyl acetate=2/1) to give methyl 6-cyano-5-methylpicolinate (30 mg, 0.17 mmol, 11%) as a white solid. MS (ESI) m/z 177.1 [M+H]⁺

Step 3. To a solution of methyl 6-cyano-5-methylpicolinate (30 mg, 0.17 mmol) in methanol (3 mL) and water (1 mL) was added sodium hydroxide (14 mg, 0.34 mmol). The mixture was stirred at room temperature for 30 min. The reaction mixture was extracted with ethyl acetate (20 mL×2) and washed with water (20 mL×2). The organic layer was evaporated to give 6-cyano-5-methylpicolinic acid (30 mg, crude) as a white solid, which was used without further purification. MS (ESI) m/z 163.1 [M+H]⁺

Step 4. A solution of 6-cyano-5-methylpicolinic (30 mg, 0.2 mmol), 6-methyl-N1-(3-(9-(tetrahydro-2H-pyran-2-yl)-9H-purin-6-yl)pyridin-2-yl)benzene-1,3-diamine (80 mg, 0.2 mmol), 1-[bis(dimethylamino)methylene]-1H-1,2,3-triazolo[4,5-b]pyridinium 3-oxid hexafluorophosphate (114 mg), N,N-diisopropylethylamine (77 mg) in N,N-dimethylformamide (3 mL) was stirred at room temperature for 2 h. The reaction mixture was extracted with ethyl acetate (50 mL×2) and washed with water (30 mL×2) to give 6-cyano-5-methyl-N-(4-methyl-3-((3-(9-(tetrahydro-2H-pyran-2-yl)-9H-purin-6-yl)pyridin-2-yl)amino)phenyl)picolinamide (90 mg, crude), which was used without further purification. MS (ESI) m/z 546.1 [M+H]⁺

Step 5. A solution of 6-cyano-5-methyl-N-(4-methyl-3-((3-(9-(tetrahydro-2H-pyran-2-yl)-9H-purin-6-yl)pyridin-2-yl)amino)phenyl)picolinamide (90 mg, 0.17 mmol), trifluoroacetic acid (56 mg, 0.5 mmol) in dichloromethane (3 mL) was stirred at room temperature for 1 h. The reaction mixture was extracted with ethyl acetate (50 mL×2) and washed with water (30 mL×2). The organic layer was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-6-cyano-5-methylpicolinamide (7.9 mg, 0.017 mmol, 11%) as a yellow solid. ¹H NMR (400 MHZ, DMSO-d₆) δ 12.29 (s, 1H), 10.46 (s, 1H), 9.78 (bs, 1H), 9.08 (s, 1H), 8.75 (d, J=1.2 Hz, 1H), 8.71 (s, 1H), 8.36 (dd, J=1.2, 3.6 Hz, 1H), 8.29 (d, J=6.4 Hz, 1H), 8.18 (d, J=6 Hz, 1H), 7.52 (dd, J=1.2, 5.2 Hz, 1H), 7.25 (d, J=6.4 Hz, 1H), 7.03 (dd, J=3.6, 6 Hz, 1H), 2.61 (s, 3H), 2.44 (s, 3H). MS (ESI) m/z 462.1 [M+H]⁺

### Example 55. Synthesis of 5-((3-(9H-purin-6-yl)pyridin-2-yl)amino)-6-methyl-N-(3-(trifluoromethyl)phenyl)nicotinamide (Compound I-52)

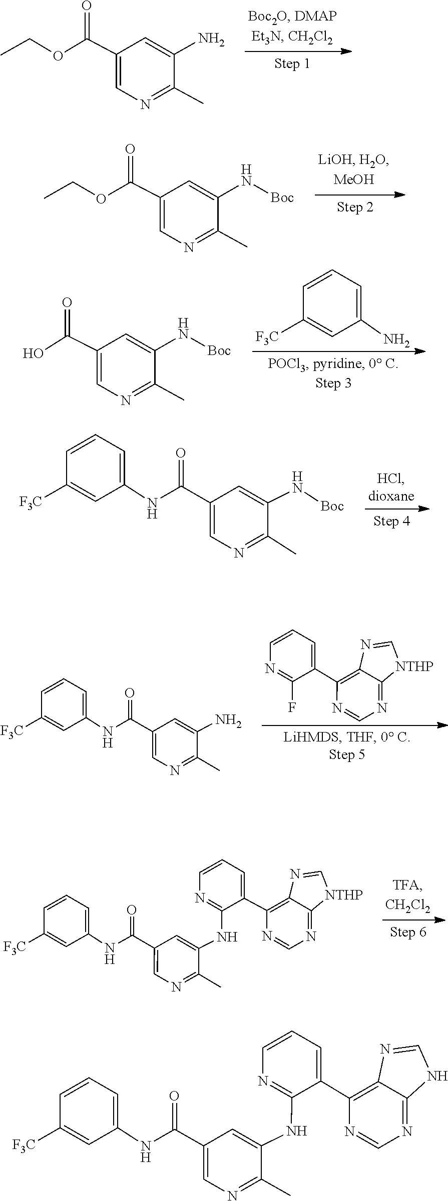

Step 1. To a solution of ethyl 5-amino-6-methylnicotinate (900 mg, 5.0 mmol) in dichloromethane (10 mL) was added 4-dimethylaminopyridine (30.5 mg, 0.25 mmol), di-tert-butyl dicarbonate (1308 mg, 6.0 mmol) and triethylamine (1010 mg, 10.0 mmol). After the addition, the reaction was stirred at 20° C. for 16 h. The mixture was concentrated and purified by column chromatography (petroleum ether/ethyl acetate=4/1) to give ethyl 5-((tert-butoxycarbonyl)amino)-6-methylnicotinate (700 mg, 1.8 mmol, 37%) as a white solid. MS (ESI) m/z 381.0 [M+H]⁺

Step 2. To a mixture of ethyl 5-((tert-butoxycarbonyl)amino)-6-methylnicotinate (700 mg, 1.8 mmol) in methanol (8 mL) and water (4 mL) was added lithium hydroxide monohydrate (155 mg, 3.6 mmol) at room temperature. Then the reaction mixture was stirred at room temperature for 2 h. The organic phase was concentrated under reduced pressure. The aqueous phase was acidified with 1N HCl until precipitation occurs. The solid was filtered and washed with water (5 mL) to give 5-((tert-butoxycarbonyl)amino)-6-methylnicotinic acid (400 mg, 1.14 mmol, 62%) as a white solid. MS (ESI) m/z 353.0 [M+H]⁺

Step 3. A mixture of 5-((tert-butoxycarbonyl)amino)-6-methylnicotinic acid (100 mg, 0.28 mmol) and 3-(trifluoromethyl)aniline (46 mg, 0.28 mmol) in anhydrous pyridine (5 mL) was added dropwise to phosphorus oxychloride (130 mg, 0.85 mmol). The mixture was stirred at 0° C. for 1 h under nitrogen. The mixture was extracted with ethyl acetate (50 mL×3). The combined organic layer was washed with brine (50 mL), dried over sodium sulfate, and filtered. The filtrate was concentrated under reduced pressure. The residue was purified by flash chromatography (petroleum ether/ethyl acetate=10/3) to give tert-butyl (2-methyl-5-((3-(trifluoromethyl)phenyl)carbamoyl)pyridin-3-yl)carbamate (135 mg, 0.27 mmol, 96%) as a yellow solid. MS (ESI) m/z 495.9 [M+H]⁺ Step 4. A solution of tert-butyl (2-methyl-5-((3-(trifluoromethyl)phenyl)carbamoyl)-pyridin-3-yl) carbamate (150 mg, 0.30 mmol) in hydrogen chloride solution (4 M in dioxane, 6 mL) was stirred at room temperature for 3 h. The mixture was concentrated and purified by column chromatography (10% methanol in dichloromethane) to give 5-amino-6-methyl-N-(3-(trifluoromethyl)phenyl)nicotinamide (70 mg, 0.24 mmol, 79%). MS (ESI) m/z 296.0 [M+H]⁺

Step 5. To a mixture of 5-amino-6-methyl-N-(3-(trifluoromethyl)phenyl)nicotinamide (70 mg, 0.24 mmol) in tetrahydrofuran (5 mL) was added 6-(2-fluoropyridin-3-yl)-9-(tetrahydro-2H-pyran-2-yl)-9H-purine (71 mg, 0.24 mmol) at 0° C. under nitrogen, followed by lithium bis(trimethylsilyl)amide (1 M, 0.71 mL, 0.71 mmol). Then the reaction mixture was stirred at 0° C. to room temperature for 2 h. The reaction mixture was diluted with water and extracted with ethyl acetate (50 mL), washed with brine (40 mL), dried over sodium sulfate, filtered and concentrated. The residue was purified by flash chromatography (ethyl acetate) to give 6-methyl-5-((3-(9-(tetrahydro-2H-pyran-2-yl)-9H-purin-6-yl)pyridin-2-yl)amino)-N-(3-(trifluoromethyl)phenyl)-nicotinamide (50 mg, 0.08 mmol, 37%) as a yellow solid. MS (ESI) m/z 574.8 [M+H]⁺

Step 6. To a mixture of 6-methyl-5-((3-(9-(tetrahydro-2H-pyran-2-yl)-9H-purin-6-yl)pyridin-2-yl)amino)-N-(3-(trifluoromethyl)phenyl)nicotinamide (45 mg, 0.078 mmol) in dichloromethane (3 mL) was added trifluoroacetic acid (3 mL) at room temperature. Then the reaction mixture was stirred at room temperature for 2 h. The reaction was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 5-((3-(9H-purin-6-yl)pyridin-2-yl) amino)-6-methyl-N-(3-(trifluoromethyl)phenyl)-nicotinamide (7.4 mg, 0.015 mmol, 19%) as a yellow solid. ¹H NMR (400 MHZ, DMSO-d₆) δ 2.79 (s, 3H), 7.14 (q, J=4.8 Hz, 1H), 7.47 (d, J=4.4 Hz, 1H), 7.62 (t, J=7.6 Hz, 1H), 8.05 (d, J=8.0 Hz, 1H), 8.27 (s, 1H), 8.42-8.43 (m, 1H), 8.71 (d, J=2.0 Hz, 1H), 8.74 (s, 1H), 9.13 (s, 1H), 9.32 (d, J=2.0 Hz, 1H), 9.88 (s, 1H), 10.67 (s, 1H), 12.72 (s, 1H). MS (ES I) m/z 490.8 [M+H]⁺

### Example 56. Synthesis of 3-((3-(9H-purin-6-yl)pyridin-2-yl)amino)-5-fluoro-4-methyl-N-(3-(trifluoromethyl)phenyl)benzamide (Compound I-53)

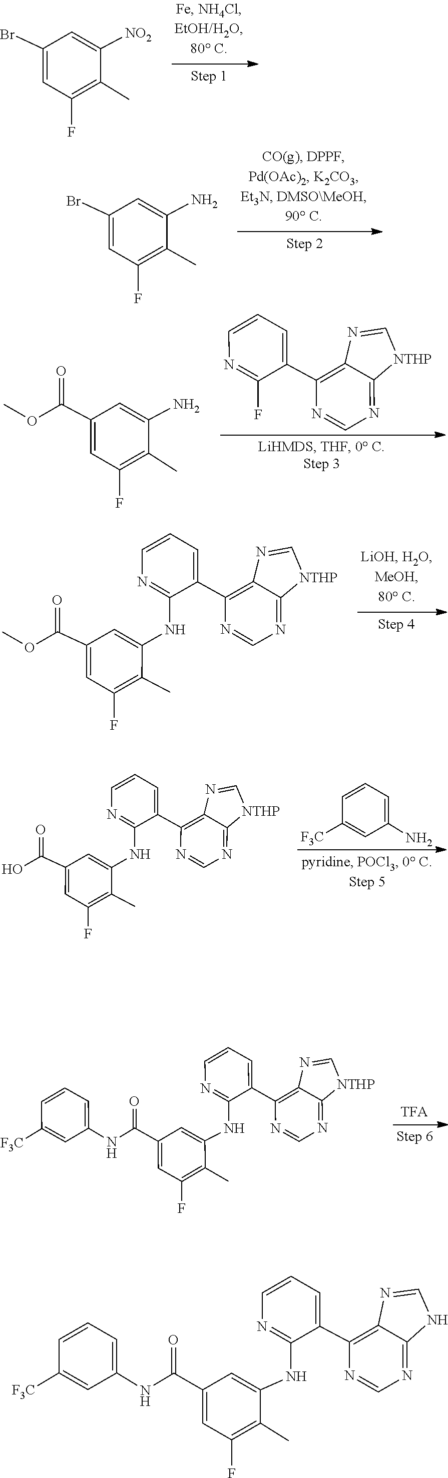

Step 1. A mixture of 5-bromo-1-fluoro-2-methyl-3-nitrobenzene (468 mg, 2.0 mmol), iron (450 mg, 8.0 mmol) and ammonium chloride (852 mg, 16.0 mmol) in ethanol (9 mL) and water (6 mL) was stirred at 80° C. for 1 h. The reaction mixture was filtered through celite and washed with ethyl acetate. The filtrate was washed with saturated sodium bicarbonate aqueous solution (50 mL) and brine (40 mL), dried over sodium sulfate, filtered and concentrated to give crude 5-bromo-3-fluoro-2-methylaniline (400 mg, 0.49 mmol) as a yellow solid. MS (ESI) m/z 204.0 [M+H]⁺

Step 2. A mixture of 5-bromo-3-fluoro-2-methylaniline (400 mg, 1.97 mmol), 1,1′-bis (diphenylphosphino)ferrocene (218 mg, 0.39 mmol), palladium (II) acetate (44 mg, 0.20 mmol) and potassium carbonate (398 mg, 3.94 mmol) in dimethyl sulfoxide (6 mL) and methanol (4 mL) was stirred at 90° C. for 16 h under carbon monoxide. The mixture was extracted with ethyl acetate (50 mL×3). The combined organic layer was washed with brine (50 mL), dried over sodium sulfate, and filtered. The filtrate was concentrated under reduced pressure. The residue was purified by flash chromatography (petroleum ether/ethyl acetate=5/1) to give methyl 3-amino-5-fluoro-4-methylbenzoate (300 mg, 1.64 mmol, 83%) as a yellow solid. MS (ESI) m/z 183.7 [M+H]⁺

Step 3. To a mixture of methyl 3-amino-5-fluoro-4-methylbenzoate (250 mg, 1.37 mmol) in tetrahydrofuran (5 mL) was added 6-(2-fluoropyridin-3-yl)-9-(tetrahydro-2H-pyran-2-yl)-9H-purine (408 mg, 1.37 mmol) at 0° C. under nitrogen, followed by lithium hexamethyldisilazide (1 M, 2.73 mL, 2.73 mmol). Then the reaction mixture was stirred at 0° C. to room temperature for 2 h. The reaction mixture was diluted with water and extracted with ethyl acetate (100 mL), washed with brine (40 mL), dried over sodium sulfate, filtered, and concentrated. The residue was purified by flash chromatography (petroleum ether/ethyl acetate=1/1) to give methyl 3-fluoro-4-methyl-5-((3-(9-(tetrahydro-2H-pyran-2-yl)-9H-purin-6-yl)pyridin-2-yl)amino)benzoate (90 mg, 0.19 mmol, 14%) as yellow solid. MS (ESI) m/z 462.9 [M+H]⁺

Step 4. To a mixture of methyl 3-fluoro-4-methyl-5-((3-(9-(tetrahydro-2H-pyran-2-yl)-9H-purin-6-yl)pyridin-2-yl)amino)benzoate (90 mg, 0.19 mmol) in methanol (4 mL) and water (2 mL) was added lithium hydroxide monohydrate (16 mg, 0.39 mmol) at room temperature. Then the reaction mixture was stirred at room temperature to 80° C. for 8 h. The organic phase was concentrated under reduced pressure. The aqueous phase was acidified with 1N HCl until precipitation occurs. The solid was filtered and washed through with water (5 mL) to give 3-fluoro-4-methyl-5-((3-(9-(tetrahydro-2H-pyran-2-yl)-9H-purin-6-yl)pyridin-2-yl) amino)benzoic acid (55 mg, 0.12 mmol, 63%) as a yellow solid. MS (ESI) m/z 448.9 [M+H]⁺

Step 5. To a mixture of 3-fluoro-4-methyl-5-((3-(9-(tetrahydro-2H-pyran-2-yl)-9H-purin-6-yl)pyridin-2-yl) amino)benzoic acid (40 mg, 0.08 mmol) and 3-fluoro-4-methyl-5-((3-(9-(tetrahydro-2H-pyran-2-yl)-9H-purin-6-yl)pyridin-2-yl)amino)-N-(3-(trifluoromethyl)phenyl)-benzamide (14 mg, 0.08 mmol) in pyridine (5 mL) was added dropwise phosphorus oxychloride (41 mg, 0.26 mmol). The mixture was stirred at 0° C. for 1 h under nitrogen. The mixture was extracted with ethyl acetate (50 mL×3). The combined organic layer was washed with brine (50 mL), dried over sodium sulfate, and filtered. The filtrate was concentrated under reduced pressure. The residue was purified by flash chromatography, (petroleum ether/ethyl acetate=1/1) to give 3-fluoro-4-methyl-5-((3-(9-(tetrahydro-2H-pyran-2-yl)-9H-purin-6-yl)pyridin-2-yl)amino)-N-(3-(trifluoromethyl)phenyl)benzamide (50 mg, 0.08 mmol, 96%) as a yellow solid. MS (ESI) m/z 591.8 [M+H]⁺

Step 6. A mixture of 3-fluoro-4-methyl-5-((3-(9-(tetrahydro-2H-pyran-2-yl)-9H-purin-6-yl)pyridin-2-yl)amino)-N-(3-(trifluoromethyl)phenyl)benzamide (45 mg, 0.076 mmol) in trifluoroacetic acid (3 mL) was stirred at room temperature for 2 h. The reaction was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 3-((3-(9H-purin-6-yl)pyridin-2-yl)amino)-5-fluoro-4-methyl-N-(3-(trifluoromethyl)-phenyl)benzamide (4.1 mg, 0.008 mmol, 11%) as a yellow solid. ¹H NMR (400 MHZ, DMSO-d₆) δ 2.44 (d, J=1.2 Hz, 3H), 7.12 (q, J=4.4 Hz, 1H), 7.46 (d, J=7.6 Hz, 1H), 7.53 (d, J=10.4 Hz, 1H), 7.61 (t, J=8.0 Hz, 1H), 8.06 (d, J=7.6 Hz, 1H), 8.26 (s, 1H), 8.40 (q, J=1.6 Hz, 1H), 8.73 (s, 1H), 8.85 (s, 1H), 9.12 (s, 1H), 9.82 (s, 1H), 10.52 (s, 1H), 12.59 (s, 1H). MS (ESI) m/z 507.9 [M+H]⁺

### Example 57. Synthesis of N-(5-(3-(9H-purin-6-yl)pyridin-2-ylamino)-6-methylpyridin-3-yl)-2-(1-cyanocyclopropyl)isonicotinamide (Compound I-54)

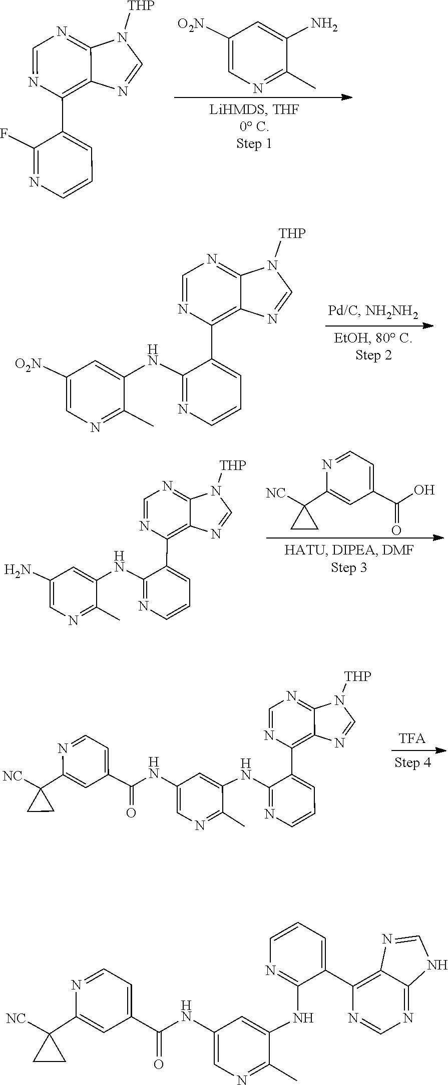

Step 1. To a solution of 2-methyl-5-nitropyridin-3-amine (512 mg, 3.34 mmol), 6-(2-fluoropyridin-3-yl)-9-(tetrahydro-2H-pyran-2-yl)-9H-purine (1.0 g, 3.34 mmol) in tetrahydrofuran (20 mL) was added lithium hexamethyldisilazide (16.7 mL, 16.7 mmol, 1 M in tetrahydrofuran) at 0° C. slowly. The mixture was stirred at 0° C. for 5 h. The reaction was quenched with saturated ammonium chloride solution (20 mL) at 0° C. and extracted with dichloromethane (50 mL×3). The combined organic layers were washed with brine (50 mL), dried over sodium sulfate, and filtered. The filtrate was concentrated under reduced pressure. The crude residue was purified by flash chromatography (methanol/dichloromethane=12/100) to give N-(2-methyl-5-nitropyridin-3-yl)-3-(9-(tetrahydro-2H-pyran-2-yl)-9H-purin-6-yl)pyridin-2-amine (600 mg, 1.39 mmol, 42%) as a yellow solid. MS (ESI) m/z 432.9 [M+H]⁺

Step 2. To a solution of N-(2-methyl-5-nitropyridin-3-yl)-3-(9-(tetrahydro-2H-pyran-2-yl)-9H-purin-6-yl)pyridin-2-amine (600 mg, 1.39 mmol) and hydrazine hydrate (85%, 3 mL) in ethanol (30 mL) was added palladium (10% on activated carbon, 300 mg). The mixture was stirred at 80° C. for 1 h. The reaction mixture was cooled, filtered, and concentrated. The residue was diluted with ethyl acetate (50 mL) and washed with water (50 mL), then brine (50 mL), dried over sodium sulfate and filtered. The filtrate was concentrated under reduced pressure. The crude residue was purified by flash chromatography (methanol/dichloromethane=10/100) to give 2-methyl-N3-(3-(9-(tetrahydro-2H-pyran-2-yl)-9H-purin-6-yl)pyridin-2-yl)pyridine-3,5-diamine (252 mg, 0.63 mmol, 45%) as a yellow solid. MS (ESI) m/z 403.0 [M+H]⁺

Step 3. A solution of 2-methyl-N3-(3-(9-(tetrahydro-2H-pyran-2-yl)-9H-purin-6-yl)pyridin-2-yl)pyridine-3,5-diamine (50 mg, 0.124 mmol), 2-(1-cyanocyclopropyl)isonicotinic acid (28 mg, 0.15 mmol), 1-[bis(dimethylamino)methylene]-1H-1,2,3-triazolo[4,5-b]pyridinium 3-oxid hexafluorophosphate (76 mg, 0.2 mmol) and N,N-diisopropylethylamine (65 mg, 0.5 mmol) in N,N-dimethylformamide (3 mL) was stirred at 25° C. for 1 h. The reaction mixture was diluted with ethyl acetate (50 mL) and washed with water (20 mL×2), brine (30 mL), dried over sodium sulfate, filtered, and concentrated. The residue was purified by flash chromatography (methanol/dichloromethane=8/100) to afford 2-(1-cyanocyclopropyl)-N-(6-methyl-5-(3-(9-(tetrahydro-2H-pyran-2-yl)-9H-purin-6-yl)pyridin-2-ylamino)pyridin-3-yl)isonicotinamide (30 mg, 0.05 mmol, 40%) as a yellow solid. MS (ESI) m/z 572.8 [M+H]⁺

Step 4. A solution of 2-(1-cyanocyclopropyl)-N-(6-methyl-5-(3-(9-(tetrahydro-2H-pyran-2-yl)-9H-purin-6-yl)pyridin-2-ylamino)pyridin-3-yl)isonicotinamide (30 mg, 0.05 mmol) in trifluoroacetic acid (1 mL) was stirred at 25° C. for 2 h. The reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(5-(3-(9H-purin-6-yl)pyridin-2-ylamino)-6-methylpyridin-3-yl)-2-(1-cyanocyclopropyl)-isonicotinamide (13.7 mg, 0.028 mmol, 56%) as a yellow solid. ¹H NMR (400 MHZ, DMSO-d₆) δ 12.68 (s, 1H), 10.71 (s, 1H), 9.87 (d, J=7.5 Hz, 1H), 9.21 (s, 1H), 9.07 (s, 1H), 8.72 (d, J=4.9 Hz, 1H), 8.68 (s, 1H), 8.58 (s, 1H), 8.39 (s, 1H), 7.96 (s, 1H), 7.84 (d, J=4.4 Hz, 1H), 7.15-7.06 (m, 1H), 2.70 (s, 3H), 1.93-1.87 (m, 2H), 1.82-1.75 (m, 2H). MS (ESI) m/z 489.1 [M+H]⁺

### Example 58. Synthesis of N-(3-(3-(9H-purin-6-yl)pyridin-2-ylamino)-4-methylphenyl)-4-cyclopropylpicolinamide (Compound I-55)

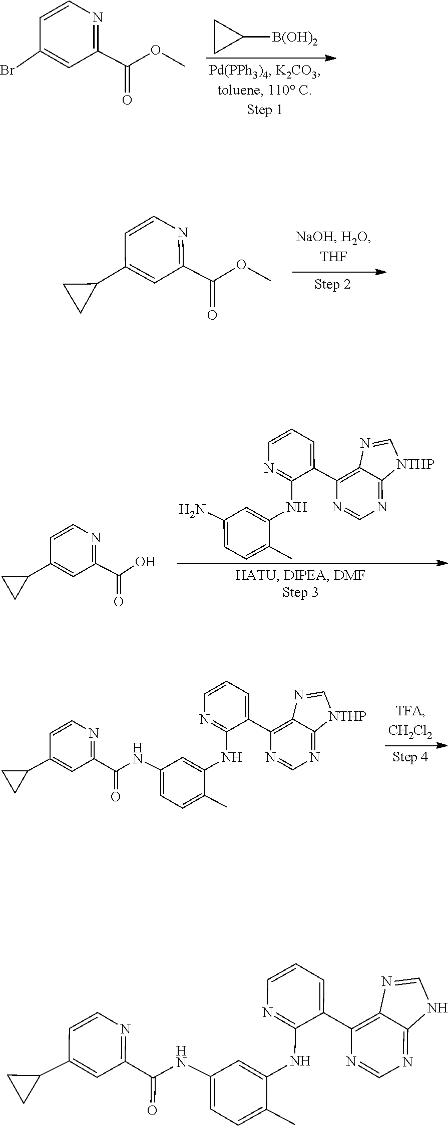

Step 1. A mixture of methyl 4-bromopicolinate (1000 mg, 4.65 mmol), cyclopropylboronic acid (2000 mg, 23.26 mmol), tetrakis(triphenylphosphine)palladium (806 mg, 0.70 mmol) and potassium carbonate (1926 mg, 13.96 mmol) in toluene (15 mL) was evacuated and refilled with nitrogen three times and stirred at 110° C. for 16 h. The mixture was filtered and concentrated. The crude residue was purified by flash chromatography (1% methanol in dichloromethane) to give methyl 4-cyclopropylpicolinate (500 mg, 2.82 mmol, 61%) as a brown oil. MS (ESI) m/z 178.2 [M+H]⁺

Step 2. To a mixture of methyl 4-cyclopropylpicolinate (500 mg, 2.82 mmol) in tetrahydrofuran (5 mL) was added a solution of sodium hydroxide (226 mg, 5.65 mmol) in water (1 mL). The mixture was stirred at room temperature for 4 h. The reaction mixture was adjusted to pH=5 with hydrochloric acid (6 N). The mixture was washed with water (20 mL), extracted with ethyl acetate (30 mL×3) and dried over sodium sulfate. The solution was filtered, concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 4-cyclopropylpicolinic acid (200 mg, 1.23 mmol, 43%) as a yellow solid. MS (ESI) m/z 164.3 [M+H]⁺

Step 3. A mixture of 4-cyclopropylpicolinic acid (30 mg, 0.18 mmol), diisopropylethylamine (48 mg, 0.37 mmol) and 1-[bis(dimethylamino)methylene]-1H-1,2,3-triazolo[4,5-b]pyridinium 3-oxid hexafluorophosphate (57 mg, 0.15 mmol) in N,N-dimethylformamide (1 mL) was stirred at room temperature for 30 min. Then 6-methyl-N1-(3-(9-(tetrahydro-2H-pyran-2-yl)-9H-purin-6-yl)pyridin-2-yl)benzene-1,3-diamine (50 mg, 0.12 mmol) was added, and the mixture was stirred at room temperature for 16 h. The reaction was washed with water (10 mL), extracted with ethyl acetate (10 mL×3) and washed with brine (10 mL×2). The organic layer was concentrated and the residue was purified by flash chromatography (5% methanol in dichloromethane) to give 4-cyclopropyl-N-(4-methyl-3-((3-(9-(tetrahydro-2H-pyran-2-yl)-9H-purin-6-yl)pyridin-2-yl)amino)phenyl)picolinamide (40 mg, 0.07 mmol, 59%) as a yellow solid. MS (ESI) m/z 547.3 [M+H]⁺

Step 4. To a mixture of 4-cyclopropyl-N-(4-methyl-3-((3-(9-(tetrahydro-2H-pyran-2-yl)-9H-purin-6-yl)pyridin-2-yl)amino)phenyl)picolinamide (40 mg, 0.07 mmol) in trifluoroacetic acid (2 mL) and dichloromethane (2 mL) was stirred at room temperature for 2 h. The mixture was concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-4-cyclopropylpicolin amide (6.1 mg, 0.013 mmol, 18%) as a yellow solid. ¹H NMR (400 MHZ, DMSO-d₆) δ 12.31 (s, 1H), 10.39 (s, 1H), 9.79 (s, 1H), 9.07 (s, 1H), 8.78 (d, J=2.0 Hz, 1H), 8.70 (s, 1H), 8.53 (d, J=5.1 Hz, 1H), 8.37 (m, J=4.7, 1.9 Hz, 1H), 7.85 (d, J=1.3 Hz, 1H), 7.52 (m, J=8.1, 2.1 Hz, 1H), 7.36 (m, J=5.1, 1.8 Hz, 1H), 7.24 (d, J=8.3 Hz, 1H), 7.04 (m, J=7.9, 4.7 Hz, 1H), 2.44 (s, 3H), 2.12 (m, J=14.3, 9.6, 5.4 Hz, 1H), 1.20-1.11 (m, 2H), 0.94-0.86 (m, 2H). MS (ESI) m/z 463.0 [M+H]⁺

### Example 59. Synthesis of N-(3-((3-(9H-purin-6-yl)pyridin-2-yl)amino)-4-methylphenyl)-5-chloro-4-cyclopropylpicolinamide (Compound I-56)

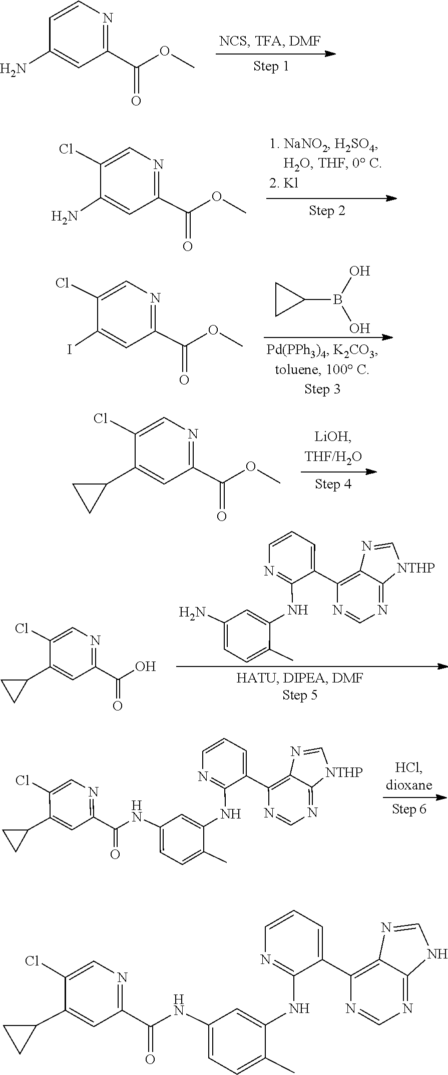

Step 1. To a mixture of methyl 4-aminopicolinate (10.00 g, 65.72 mmol) and trifluoroacetic acid (1 mL) in N, N-dimethylformamide (200 mL) was added N-chlorosuccinimide (9.65 g, 72.30 mmol) at 0° C. The mixture was stirred at room temperature for 3 h and poured into water (500 mL). The mixture was extracted with ethyl acetate (200 mL×3). The combined organic phases were washed with brine, dried over sodium sulfate, filtered, and concentrated. The residue was purified by flash chromatograph on silica gel (petroleum ether/ethyl acetate=10/1) to give methyl 4-amino-5-chloropicolinate (3.90 g, 20.90 mmol, 32%) as a yellow solid. MS (ESI) m/z 187.2 [M+H]⁺

Step 2. Sodium nitrite (2.82 g, 40.86 mmol) was added into a solution of methyl 4-amino-5-chloropicolinate (1.90 g, 10.22 mmol) and concentrated sulfuric acid (5 mL) in water (20 mL) and tetrahydrofuran (20 mL) at 0° C. The mixture was stirred at 0° C. for 1.5 h, and potassium iodide (6.78 g, 40.86 mmol) was added. The mixture was stirred at room temperature for 16 h. The resulting mixture was concentrated under reduced pressure and extracted with ethyl acetate (50 mL×3). The combined organic phases were washed with saturated sodium sulfite solution (50 mL), then with brine, dried over sodium sulfate, and filtered. The filtrate was concentrated and the residue was purified by flash chromatograph on silica gel (petroleum ether/ethyl acetate=10/1) to give methyl 5-chloro-4-iodopicolinate (750 mg, 2.53 mmol, 25%) as a white solid. MS (ESI) m/z 297.9 [M+H]⁺

Step 3. To a mixture of methyl 5-chloro-4-iodopicolinate (300 mg, 1.01 mmol) in toluene (5 mL) was added cyclopropylboronic acid (260 mg, 3.03 mmol), tetrakis(triphenylphosphine)-palladium (175 mg, 0.15 mmol), and potassium carbonate (418 mg, 3.03 mmol). The mixture was stirred at 100° C. for 16 h and allowed to cool to room temperature. The resulting mixture was filtered and the filtrate was concentrated under reduced pressure. The residue was purified by flash chromatograph on silica gel (petroleum ether/ethyl acetate=4/1) to give methyl 5-chloro-4-cyclopropylpicolinate (180 mg, 0.85 mmol, 85%) as a white solid. MS (ESI) m/z 212.1 [M+H]⁺

Step 4. To a mixture of methyl 5-chloro-4-cyclopropylpicolinate (180 mg, 0.85 mmol) in tetrahydrofuran (2 mL) and water (2 mL) was added lithium hydroxide (102 mg, 4.25 mmol). The mixture was stirred at room temperature for 1 h. Tetrahydrofuran was removed under reduced pressure and the mixture was adjusted to pH=3 by adding 1 N hydrochloric acid slowly. The mixture was extracted with ethyl acetate (10 mL×3). The combined organic phases were washed with brine, dried over sodium sulfate, filtered, and concentrated to give 5-chloro-4-cyclopropylpicolinic acid (150 mg, crude) as a white solid. MS (ESI) m/z 197.9 [M+H]⁺

Step 5. To a mixture of 5-chloro-4-cyclopropylpicolinic acid (50 mg, crude) in N,N-dimethylformamide (2 mL) was added 1-[bis(dimethylamino)methylene]-1H-1,2,3-triazolo[4,5-b]pyridinium 3-oxid hexafluorophosphate (116 mg, 0.30 mmol) and N,N-diisopropylethylamine (49 mg, 0.38 mmol) at room temperature. After stirring for 5 min, 6-methyl-N1-(3-(9-(tetrahydro-2H-pyran-2-yl)-9H-purin-6-yl)pyridin-2-yl)benzene-1,3-diamine (102 mg, 0.25 mmol) was added. The mixture was stirred at room temperature for another 3 h. The resulting mixture was purified by prep-TLC (silica, petroleum ether/ethyl acetate=1/1) to give 5-chloro-4-cyclopropyl-N-(4-methyl-3-((3-(9-(tetrahydro-2H-pyran-2-yl)-9H-purin-6-yl)pyridin-2-yl)amino)phenyl)-picolinamide (30 mg, 0.05 mmol, 6% yield for 2 steps) as a yellow solid. MS (ESI) m/z 580.9 [M+H]⁺

Step 6. A solution of 5-chloro-4-cyclopropyl-N-(4-methyl-3-((3-(9-(tetrahydro-2H-pyran-2-yl)-9H-purin-6-yl)pyridin-2-yl)amino)phenyl)picolinamide (30 mg, 0.05 mmol) in hydrogen chloride (4 M in dioxane, 2 mL) was stirred at room temperature for 1 h. The solvent was removed under reduced pressure and the residue was treated with ammonia (1 mL, 8 M in methanol). The resulting mixture was filtered and the solid was collected. The solid was dried under reduced pressure to give N-(3-((3-(9H-purin-6-yl)pyridin-2-yl)amino)-4-methylphenyl)-5-chloro-4-cyclopropylpicolinamide (4.4 mg, 0.009 mmol, 18%) as a yellow solid. ¹H NMR (400 MHZ, DMSO-d₆) δ 12.32 (s, 1H), 10.41 (s, 1H), 9.78 (d, J=7.2 Hz, 1H), 9.06 (s, 1H), 8.78 (s, 1H), 8.69 (d, J=6.4 Hz, 2H), 8.35 (d, J=4.4 Hz, 1H), 7.63 (s, 1H), 7.49 (d, J=7.6 Hz, 1H), 7.23 (d, J=8.0 Hz, 1H), 7.05-7.02 (m, 1H), 2.43 (s, 3H), 2.34-2.27 (m, 1H), 1.26-1.21 (m, 2H), 0.99-0.96 (m, 2H). MS (ESI) m/z 496.8 [M+H]⁺

### Example 60. Synthesis of N-(6-((3-(9H-purin-6-yl)pyridin-2-yl)amino)-5-methylpyridin-2-yl)-3-(2-cyanopropan-2-yl)benzamide (Compound I-57)

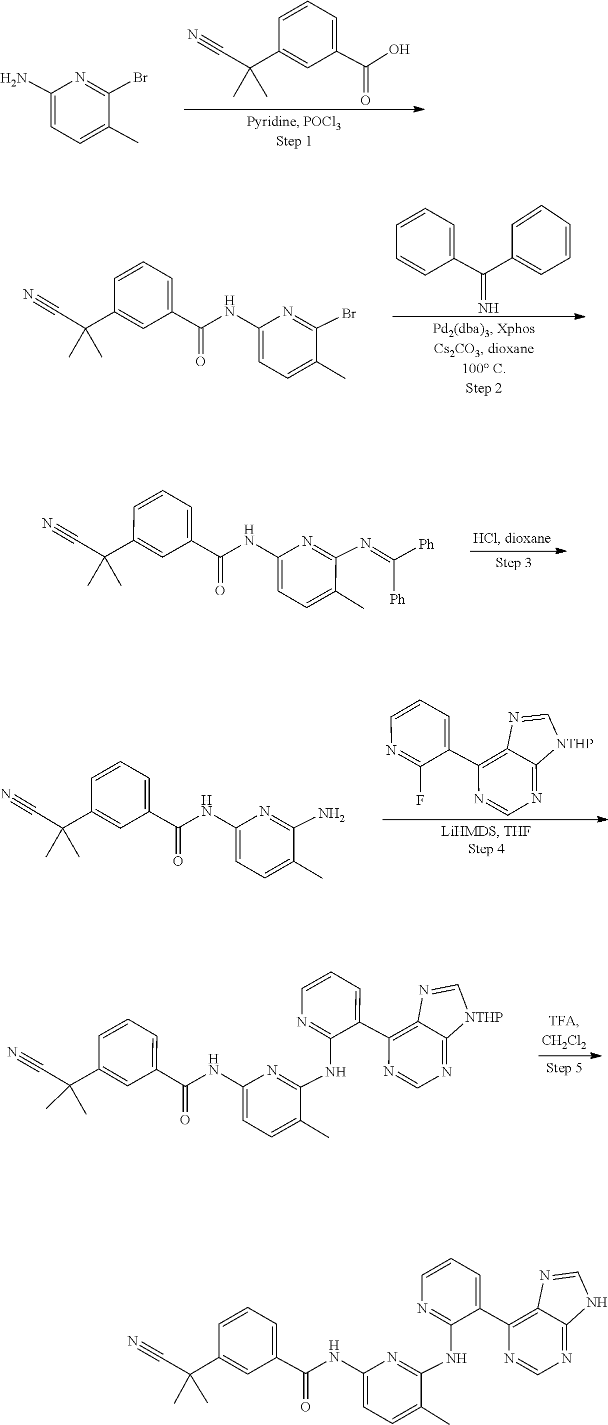

Step 1. To a mixture of 3-(2-cyanopropan-2-yl)benzoic acid (202 mg, 1.07 mmol) in pyridine was added 6-bromo-5-methylpyridin-2-amine (200 mg, 1.07 mmol) at 0° C. under nitrogen atmosphere. Then phosphorus(V) oxychloride (490 mg, 3.21 mmol) was added dropwise. The mixture was stirred at room temperature for 16 h. The mixture was concentrated in vacuo. The residue was diluted with saturated sodium bicarbonate aqueous solution (30 mL) and extracted with ethyl acetate (30 mL×3). The combined organic phases were concentrated in vacuo to yield N-(6-bromo-5-methylpyridin-2-yl)-3-(2-cyanopropan-2-yl)benzamide (240 mg, 0.67 mmol, 63%). MS (ESI) m/z 358.9 [M+H]⁺

Step 2. A mixture of N-(6-bromo-5-methylpyridin-2-yl)-3-(2-cyanopropan-2-yl)benzamide (240 mg, 0.67 mmol), diphenylmethanimine (183 mg, 1.01 mmol), tris(dibenzylideneacetone)dipalladium (62 mg, 0.07 mmol), 2-dicyclohexylphosphino-2′,4′,6′-triisopropylbiphenyl (106 mg, 0.2 mmol) and cesium carbonate (659 mg, 2.02 mmol) in dioxane (5 mL) was stirred at 100° C. for 16 h. The mixture was concentrated in vacuo. The residue was diluted with water (30 mL) and extracted with ethyl acetate (30 mL×3). The combined organic phases were concentrated in vacuo. The residue was purified by silica gel column chromatography (ethyl acetate/petroleum ether=0% to 33%) to afford 3-(2-cyanopropan-2-yl)-N-(6-((diphenylmethylene)amino)-5-methylpyridin-2-yl)benzamide (135 mg, 0.29 mmol, 44%). MS (ESI) m/z 459.1 [M+H]⁺

Step 3. A mixture of 3-(2-cyanopropan-2-yl)-N-(6-((diphenylmethylene)amino)-5-methylpyridin-2-yl)benzamide (135 mg, 0.29 mmol) in hydrogen chloride (4 M in dioxane, 2 mL) was stirred at room temperature for 2 h. The mixture was concentrated in vacuo. The residue was diluted with water (20 mL) and extracted with ethyl acetate (20 mL×3). The combined organic phases were concentrated in vacuo. The residue was purified by silica gel column chromatography (ethyl acetate/petroleum ether=0% to 30%) to afford N-(6-amino-5-methylpyridin-2-yl)-3-(2-cyanopropan-2-yl)benzamide (75 mg, 0.26 mmol, 68%). MS (ESI) m/z 295.2 [M+H]⁺

Step 4. A mixture of N-(6-amino-5-methylpyridin-2-yl)-3-(2-cyanopropan-2-yl)benzamide (75.0 mg, 0.26 mmol) and 6-(2-fluoropyridin-3-yl)-9-(tetrahydro-2H-pyran-2-yl)-9H-purine (114.4 mg, 0.38 mmol) in tetrahydrofuran (2 mL) was stirred at 0° C. while lithium hexamethyldisilazide (85 mg, 0.38 mmol) was added dropwise over 5 min. Then the mixture was stirred at room temperature for 16 h. The residue was filtered and concentrated in vacuo to afford 3-(2-cyanopropan-2-yl)-N-(5-methyl-6-((3-(9-(tetrahydro-2H-pyran-2-yl)-9H-purin-6-yl)pyridin-2-yl)amino) pyridin-2-yl)benzamide (22 mg, 0.04 mmol, 15%). MS (ESI) m/z 574.2 [M+H]⁺

Step 5. 3-(2-cyanopropan-2-yl)-N-(5-methyl-6-((3-(9-(tetrahydro-2H-pyran-2-yl)-9H-purin-6-yl)pyridin-2-yl)amino)pyridin-2-yl)benzamide (22 mg, 0.04 mmol) was dissolved in trifluoroacetic acid/dichloromethane (1:1, 2 mL) and stirred at room temperature for 16 h. The mixture was concentrated in vacuo. The residue was diluted with saturated sodium bicarbonate aqueous solution (20 mL) and extracted with ethyl acetate (3×20 mL). The combined organic phases were concentrated in vacuo to afford N-(6-((3-(9H-purin-6-yl)pyridin-2-yl)amino)-5-methylpyridin-2-yl)-3-(2-cyanopropan-2-yl)benzamide (16.9 mg, 0.034 mmol, 90%) as a yellow solid. ¹H NMR (400 MHZ, DMSO-d₆) δ 11.74 (s, 1H), 10.87 (s, 1H), 9.50 (d, J=6.8 Hz, 1H), 8.99 (s, 1H), 8.23 (s, 1H), 8.25-8.22 (m, 2H), 8.16-8.15 (m, 1H), 7.98-7.95 (m, 2H), 7.74-7.71 (m, 2H), 7.56 (t, J=8 Hz, 1H), 7.04-7.01 (m, 1H), 2.16 (s, 3H), 1.74 (s, 6H). MS (ESI) m/z 490.1 [M+H]⁺

### Example 61. Synthesis of 5-((3-(9H-purin-6-yl)pyridin-2-yl)amino)-N-(3-(1-cyanocyclopropyl)phenyl)-6-methylnicotinamide (Compound I-58)

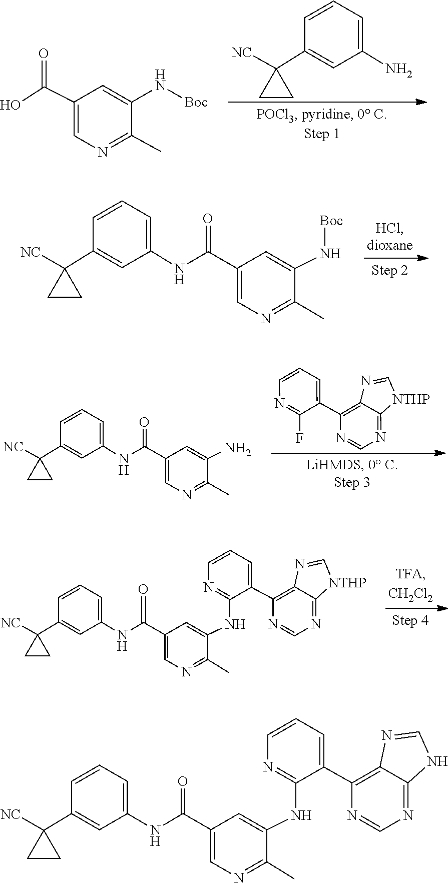

Step 1. To a mixture of 5-((tert-butoxycarbonyl)amino)-6-methylnicotinic acid (150 mg, 0.60 mmol) and 1-(3-aminophenyl)cyclopropane-1-carbonitrile (94 mg, 0.60 mmol) in pyridine (5 mL) was added dropwise phosphorus(V) oxychloride (273 mg, 1.79 mmol). The mixture was stirred at 0° C. for 1 h under nitrogen. The mixture was extracted with ethyl acetate (50 mL×3). The combined organic layer was washed with brine (50 mL), dried over sodium sulfate and filtered. The filtrate was concentrated under reduced pressure. The residue was purified by flash chromatography (petroleum ether/ethyl acetate=5/3) to give tert-butyl(5-((3-(1-cyanocyclopropyl)phenyl)carbamoyl)-2-methylpyridin-3-yl)carbamate (200 mg, 0.51 mmol, 87%) as a yellow solid. MS (ESI) m/z 393.0 [M+H]⁺

Step 2. A mixture of tert-butyl(5-((3-(1-cyanocyclopropyl)phenyl)carbamoyl)-2-methylpyridin-3-yl)carbamate (200 mg, 0.51 mmol) and hydrogen chloride solution (4 M in dioxane, 6 mL) was stirred at room temperature for 3 h. The mixture was concentrated and purified by flash chromatography (10% methanol in dichloromethane) to give 5-amino-N-(3-(1-cyanocyclopropyl)phenyl)-6-methylnicotinamide (100 mg, 034 mmol, 67%) as a solid. MS (ESI) m/z 293.0 [M+H]⁺

Step 3. To a mixture of 5-amino-N-(3-(1-cyanocyclopropyl)phenyl)-6-methylnicotinamide (100 mg, 0.34 mmol) in tetrahydrofuran (5 mL) was added 6-(2-fluoropyridin-3-yl)-9-(tetrahydro-2H-pyran-2-yl)-9H-purine (102 mg, 0.34 mmol) at 0° C. under nitrogen, followed by lithium hexamethyldisilazide (1 M in tetrahydrofuran, 1.03 mL, 1.03 mmol). Then the reaction mixture was stirred at 0° C. to room temperature for 2 h. The reaction mixture was diluted with water and extracted with ethyl acetate (50 mL). The combined organic layer was washed with brine (40 mL), dried over sodium sulfate, filtered and concentrated. The residue was purified by flash chromatography (ethyl acetate=100%) to give N-(3-(1-cyanocyclopropyl)phenyl)-6-methyl-5-((3-(9-(tetrahydro-2H-pyran-2-yl)-9H-purin-6-yl)pyridin-2-yl)amino)nicotinamide (80 mg, 0.14 mmol, 41%) as a yellow solid. MS (ESI) m/z 571.7 [M+H]⁺

Step 4. To a mixture of N-(3-(1-cyanocyclopropyl)phenyl)-6-methyl-5-((3-(9-(tetrahydro-2H-pyran-2-yl)-9H-purin-6-yl)pyridin-2-yl)amino)nicotinamide (70 mg, 0.123 mmol) in dichloromethane (3 mL) was added trifluoroacetic acid (3 mL) at room temperature. Then the reaction mixture was stirred at room temperature for 2 h. The reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 5-((3-(9H-purin-6-yl)pyridin-2-yl)amino)-N-(3-(1-cyanocyclopropyl)phenyl)-6-methylnicotinamide (13.9 mg, 0.029 mmol, 24%) as a yellow solid. ¹H NMR (400 MHZ, DMSO-d₆) δ 1.48-1.51 (m, 2H), 1.76-1.80 (m, 2H), 2.79 (s, 3H), 7.02 (d, J=6.8 Hz, 1H), 7.14 (q, J=4.8 Hz, 1H), 7.38 (t, J=8.0 Hz, 1H), 7.77 (d, J=7.2 Hz, 1H), 7.88 (s, 1H), 8.42-8.44 (m, 1H), 8.69 (d, J=2.0 Hz, 1H), 8.75 (s, 1H), 9.13 (s, 1H), 9.30 (s, 1H), 9.90 (d, J=4.0 Hz, 1H), 10.46 (s, 1H), 12.72 (s, 1H), 13.91 (s, 1H). MS (ESI) m/z 488.0 [M+H]⁺

### Example 62. Synthesis of N-(3-((3-(9H-purin-6-yl)pyridin-2-yl)amino)-4-methylphenyl)-6-cyano-5-(trifluoromethyl)picolinamide (Compound I-59)

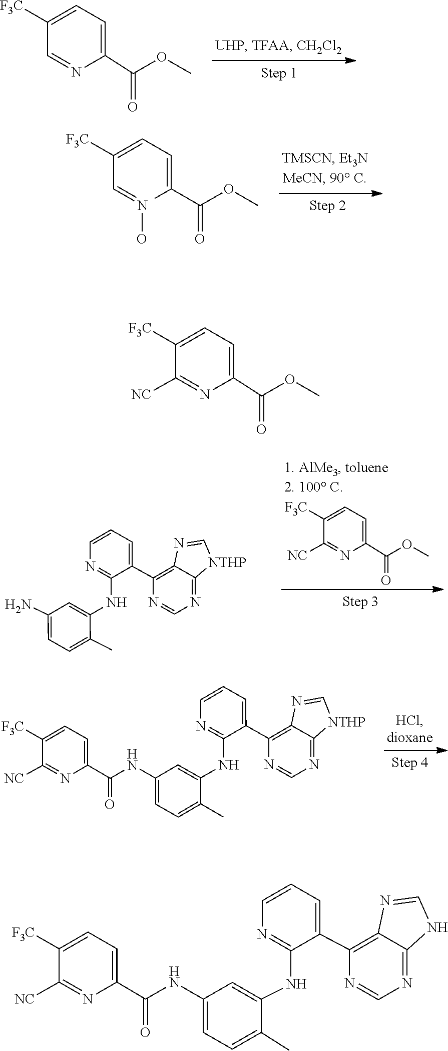

Step 1. To a mixture of methyl 5-(trifluoromethyl)picolinate (1.20 g, 5.85 mmol) and urea hydrogen peroxide adduct (1.1 g, 11.70 mmol) in dichloromethane (50 mL) was added trifluoroacetic anhydride (2.45 g, 11.70 mmol) at 0° C. The mixture was stirred at room temperature for 16 h, and then quenched by addition of saturated sodium sulfite aqueous solution (100 mL). The organic phase was separated and washed with saturated sodium bicarbonate solution (50 mL), dried over sodium sulfate, filtered and concentrated. The residue was purified by flash chromatograph on silica gel (petroleum ether/ethyl acetate=1/1) to give 2-(methoxycarbonyl)-5-(trifluoromethyl)pyridine 1-oxide (860 mg, 3.89 mmol, 67%) as a white solid. MS (ESI) m/z 222.1 [M+H]⁺

Step 2. Trimethylsilyl cyanide (372 mg, 3.75 mmol) was added into a solution of 2-(methoxycarbonyl)-5-(trifluoromethyl)pyridine 1-oxide (400 mg, 1.50 mmol), triethylamine (372 mg, 3.75 mmol) in acetonitrile (5 mL) at room temperature. The mixture was stirred at 90° C. for 16 h. The resulting mixture was concentrated under reduced pressure and the residue was then redissolved in dichloromethane (20 mL). The solution was washed with saturated sodium sulfite solution (20 mL) and brine (10 mL), dried over sodium sulfate, and filtered. The mixture was concentrated and the residue was purified by flash chromatograph on silica gel (petroleum ether/ethyl acetate=2/1) to give methyl 6-cyano-5-(trifluoromethyl)picolinate (160 mg, 0.70 mmol, 46.7%) as a yellow solid. ¹H NMR (400 MHZ, DMSO-d₆) δ 8.58-8.51 (m, 2H), 4.05 (s, 3H)

Step 3. To a mixture of 6-methyl-N1-(3-(9-(tetrahydro-2H-pyran-2-yl)-9H-purin-6-yl)pyridin-2-yl)benzene-1,3-diamine (100 mg, 0.25 mmol) in toluene (5 mL) was added trimethylaluminium (1.0 mL, 1.0 mmol, 1 M in hexanes) at 0° C. The mixture was stirred at room temperature for 2 h, then methyl 6-cyano-5-(trifluoromethyl)picolinate (140 mg, 0.61 mmol) dissolved in toluene (1 mL) was added. The mixture was stirred at 100° C. for 3 h and allowed to cool to room temperature. The resulting mixture was concentrated under reduced pressure. The residue was diluted with ethyl acetate and filtered. The filtrate was concentrated and purified by prep-TLC (petroleum ether/ethyl acetate=1/1) to give 6-cyano-N-(4-methyl-3-((3-(9-(tetrahydro-2H-pyran-2-yl)-9H-purin-6-yl)pyridin-2-yl)amino)phenyl)-5-(trifluoromethyl)picolinamide (35 mg, 0.06 mmol, 23%) as a yellow solid. MS (ESI) m/z 600.0 [M+H]⁺

Step 4. A solution of 6-cyano-N-(4-methyl-3-((3-(9-(tetrahydro-2H-pyran-2-yl)-9H-purin-6-yl)pyridin-2-yl)amino)phenyl)-5-(trifluoromethyl)picolinamide (30 mg, 0.05 mmol) in hydrogen chloride solution (4 M in dioxane, 2 mL) was stirred at room temperature for 1 h. The solvent was removed under reduced pressure and the residue was treated with ammonia (1 mL, 8 M in methanol). The resulting mixture was filtered and the solid was collected. The solid was dried under reduced pressure to give N-(3-((3-(9H-purin-6-yl)pyridin-2-yl)amino)-4-methylphenyl)-6-cyano-5-(trifluoromethyl)picolinamide (2.1 mg, 0.004 mmol, 8%) as a yellow solid. ¹H NMR (400 MHZ, DMSO-d₆) δ 13.88 (s, 1H), 12.36 (s, 1H), 10.74 (s, 1H), 9.83 (d, J=8.4 Hz, 1H), 9.09 (s, 1H), 8.80 (s, 1H), 8.73-8.71 (m, 2H), 8.55 (d, J=8.4 Hz, 1H), 8.37 (d, J=4.4 Hz, 1H), 7.53 (d, J=9.6 Hz, 1H), 7.28 (d, J=8.8 Hz, 1H), 7.06-7.03 (m, 1H), 6.53 (s, 0.15H), 2.46 (s, 3H). MS (ESI) m/z 516.0 [M+H]⁺

### Example 63. Synthesis of 3-((3-(9H-purin-6-yl)pyridin-2-yl)amino)-N-(3-(1-cyanocyclopropyl)phenyl)-5-fluoro-4-methylbenzamide (Compound I-60)

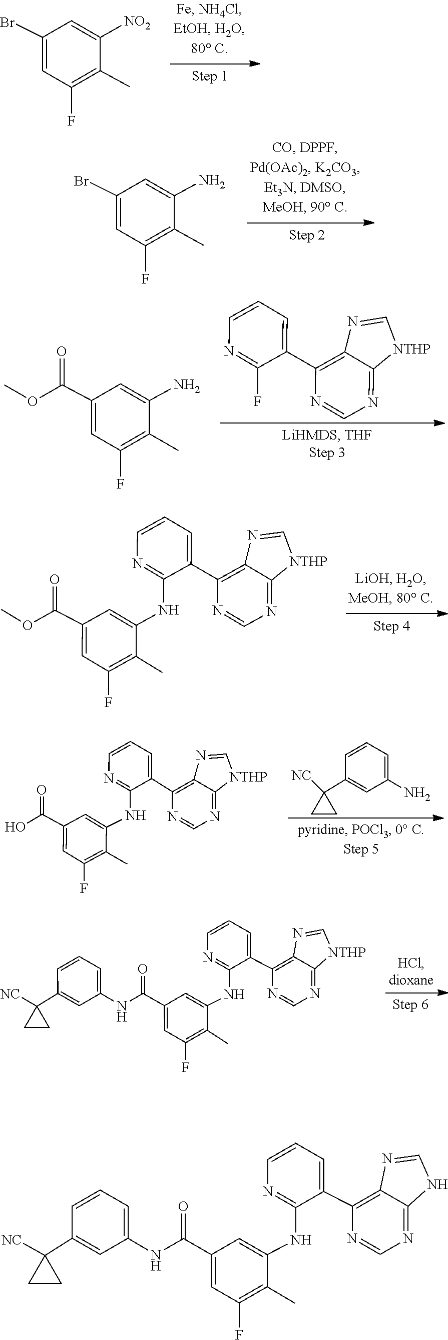

Step 1. A mixture of 5-bromo-1-fluoro-2-methyl-3-nitrobenzene (468 mg, 2.0 mmol), iron (450 mg, 8.0 mmol) and ammonium chloride (852 mg, 16.0 mmol) in ethanol (9 mL) and water (6 mL) was stirred at 80° C. for 1 h. The reaction mixture was filtered through celite and washed with ethyl acetate. The filtrate was washed with saturated sodium bicarbonate aqueous solution (50 mL) and brine (40 mL), dried over sodium sulfate, filtered and concentrated to give crude 5-bromo-3-fluoro-2-methylaniline (400 mg, 0.49 mmol) as a yellow solid. MS (ESI) m/z 204.0 [M+H]⁺

Step 2. A mixture of 5-bromo-3-fluoro-2-methylaniline (400 mg, 1.97 mmol), 1,1′-bis (diphenylphosphino)ferrocene (218 mg, 0.39 mmol), palladium (II) acetate (44 mg, 0.20 mmol) and potassium carbonate (398 mg, 3.94 mmol) in dimethyl sulfoxide (6 mL) and methanol (4 mL) was stirred at 90° C. for 16 h under carbon monoxide. The mixture was extracted with ethyl acetate (50 mL×3). The combined organic layer was washed with brine (50 mL), dried over sodium sulfate, and filtered. The filtrate was concentrated under reduced pressure. The residue was purified by flash chromatography (petroleum ether/ethyl acetate=5/1) to give methyl 3-amino-5-fluoro-4-methyl benzoate (200 mg, 1.09 mmol, 56%) as a yellow solid. MS (ESI) m/z 184.1 [M+H]⁺

Step 3. To a mixture of methyl 3-amino-5-fluoro-4-methylbenzoate (200 mg, 1.09 mmol) in tetrahydrofuran (5 mL) was added 6-(2-fluoropyridin-3-yl)-9-(tetrahydro-2H-pyran-2-yl)-9H-purine (327 mg, 1.09 mmol) at 0° C. under nitrogen, followed by lithium hexamethyldisilazide (1 M in tetrahydrofuran, 2.18 mL, 2.18 mmol). Then the reaction mixture was stirred at 0° C. to room temperature for 2 h. The reaction mixture was diluted with water and extracted with ethyl acetate (100 mL), washed with brine (40 mL), dried over sodium sulfate, filtered, and concentrated. The residue was purified by flash chromatography (petroleum ether/ethyl acetate=1/1) to give methyl-3-fluoro-4-methyl-5-((3-(9-(tetrahydro-2H-pyran-2-yl)-9H-purin-6-yl)pyridin-2-yl)amino)benzoate (110 mg, 0.24 mmol, 22%) as a yellow solid. MS (ESI) m/z 462.9 [M+H]⁺

Step 4. To a mixture of methyl-3-fluoro-4-methyl-5-((3-(9-(tetrahydro-2H-pyran-2-yl)-9H-purin-6-yl) pyridin-2-yl)amino)benzoate (110 mg, 0.24 mmol) in methanol (4 mL) and water (2 mL) was added lithium hydroxide monohydrate (20 mg, 0.48 mmol) at room temperature. Then the reaction mixture was stirred at 80° C. for 8 h. The mixture was concentrated under reduced pressure. The residual aqueous phase was acidified with 1 N HCl until precipitation occurs. The solid was filtered and washed with water (5 mL) to give 3-fluoro-4-methyl-5-((3-(9-(tetrahydro-2H-pyran-2-yl)-9H-purin-6-yl)pyridin-2-yl)amino)benzoic acid (60 mg, 0.13 mmol, 57%) as a yellow solid. MS (ESI) m/z 449.0 [M+H]⁺

Step 5. To a mixture of 3-fluoro-4-methyl-5-((3-(9-(tetrahydro-2H-pyran-2-yl)-9H-purin-6-yl)pyridin-2-yl)amino)benzoic acid (50 mg, 0.11 mmol) and 1-(3-aminophenyl)cyclopropane-1-carbonitrile (18 mg, 0.11 mmol) in pyridine (5 mL) was added dropwise phosphorus(V) oxychloride (51 mg, 0.33 mmol). The mixture was stirred at 0° C. for 1 h under nitrogen. The mixture was extracted with ethyl acetate (50 mL×3). The combined organic layer was washed with brine (50 mL), dried over sodium sulfate, and filtered. The filtrate was concentrated under reduced pressure. The residue was purified by flash chromatography (petroleum ether/ethyl acetate=1/1) to give N-(3-(1-cyanocyclopropyl)phenyl)-3-fluoro-4-methyl-5-((3-(9-(tetrahydro-2H-pyran-2-yl)-9H-purin-6-yl)pyridin-2-yl)amino)benzamide (25 mg, 0.04 mmol, 38%) as a yellow solid. MS (ESI) m/z 588.9 [M+H]⁺

Step 6. To a solution of N-(3-(1-cyanocyclopropyl)phenyl)-3-fluoro-4-methyl-5-((3-(9-(tetrahydro-2H-pyran-2-yl)-9H-purin-6-yl)pyridin-2-yl)amino)benzamide (25 mg, 0.04 mmol) in methanol (2 mL) was added hydrogen chloride solution (4 M in dioxane, 3 mL) at room temperature, and the mixture was stirred at room temperature for 1 h. After completed, the reaction mixture was concentrated and methanol (3 mL) was added. The pH was adjusted to 8 with ammonia (7 N in methanol). The mixture concentrated under reduced pressure. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 3-((3-(9H-purin-6-yl)pyridin-2-yl)amino)-N-(3-(1-cyanocyclopropyl)phenyl)-5-fluoro-4-methylbenzamide (2.3 mg, 0.005 mmol, 11%) as a yellow solid. ¹H NMR (400 MHZ, DMSO-d₆) δ 1.47-1.50 (m, 2H), 1.77 (q, J=5.0 Hz, 2H), 2.43 (s, 3H), 7.01 (d, J=7.5 Hz, 1H), 7.11 (q, J=4.5 Hz, 1H), 7.36 (t, J=8.0 Hz, 1H), 7.51 (d, J=10.5 Hz, 1H), 7.77 (d, J=9.0 Hz, 1H), 7.86 (s, 1H), 8.40 (q, J=2.0 Hz, 1H), 8.73 (s, 1H), 8.82 (s, 1H), 9.11 (s, 1H), 9.82 (s, 1H), 10.31 (s, 1H), 12.58 (s, 1H). MS (ESI) m/z 504.9 [M+H]⁺

### Example 64. Synthesis of N-(5-((3-(9H-purin-6-yl)pyridin-2-yl)amino)-6-methylpyridin-3-yl)-2-(1-fluorocyclopropyl)isonicotinamide (Compound I-61)

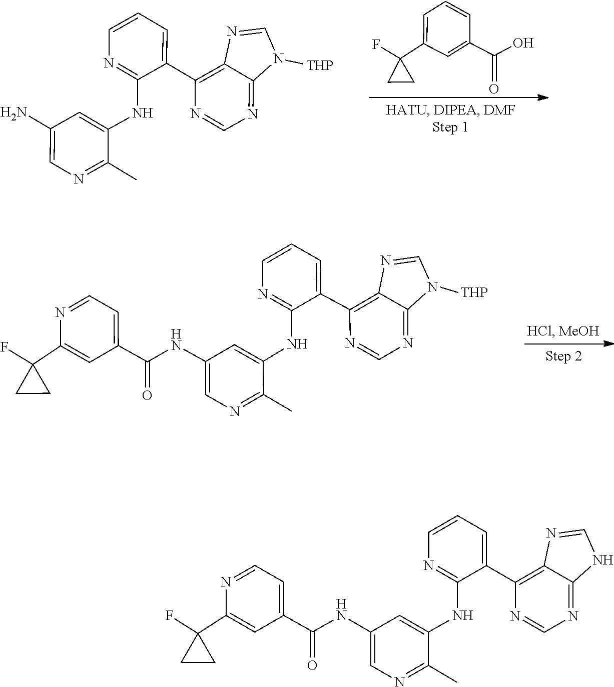

Step 1. A mixture of 2-methyl-N3-(3-(9-(tetrahydro-2H-pyran-2-yl)-9H-purin-6-yl)pyridin-2-yl)pyridine-3,5-diamine (67 mg, 0.16 mmol), 3-(1-fluorocyclopropyl)benzoic acid (30 mg, 0.16 mmol), N,N-diisopropylethylamine (41 mg, 0.32 mmol) and 1-[bis(dimethylamino)methylene]-1H-1,2,3-triazolo[4,5-b]pyridinium 3-oxid hexafluorophosphate (91 mg, 0.24 mmol) in N,N-dimethylformamide (5 mL) was stirred at room temperature for 1 h. The resulting mixture was poured into water and extracted with ethyl acetate (10 mL×3). The combined organic phases were washed with brine, dried over sodium sulfate, filtered, concentrated and purified by flash chromatography (80% ethyl acetate in petroleum ether) to give 2-(1-fluorocyclopropyl)-N-(6-methyl-5-((3-(9-(tetrahydro-2H-pyran-2-yl)-9H-purin-6-yl)pyridin-2-yl)amino)pyridin-3-yl)isonicotinamide (40 mg, 0.07 mmol, 44%) as a yellow solid. MS (ESI) m/z 565.8 [M+H]⁺

Step 2. A mixture of 2-(1-fluorocyclopropyl)-N-(6-methyl-5-((3-(9-(tetrahydro-2H-pyran-2-yl)-9H-purin-6-yl)pyridin-2-yl)amino)pyridin-3-yl)isonicotinamide (40 mg, 0.07 mmol) in hydrogen chloride solution (3 M in methanol, 10 mL) was stirred at room temperature for 3 h. The resulting mixture was concentrated, quenched with ammonia solution in methanol and concentrated again. The residue was triturated with methanol and dried to give N-(5-((3-(9H-purin-6-yl)pyridin-2-yl)amino)-6-methylpyridin-3-yl)-2-(1-fluorocyclopropyl)isonicotinamide (8.0 mg, 0.017 mmol, 24%) as a light yellow solid. ¹H NMR (500 MHZ, CD₃OD-d₆) δ 13.90 (s, 1H), 12.61 (s, 1H), 10.73 (s, 1H), 9.87 (s, 1H), 9.22 (s, 1H), 9.12 (s, 1H), 8.80-8.68 (m, 2H), 8.61 (d, J=2.1 Hz, 1H), 8.41 (dd, J=4.7, 1.8 Hz, 1H), 8.12 (s, 1H), 7.82 (d, J=5.0 Hz, 1H), 7.12 (dd, J=7.9, 4.7 Hz, 1H), 2.70 (s, 3H), 1.66-1.55 (m, 2H), 1.46-1.38 (m, 2H). MS (ESI) m/z 482.1 [M+H]⁺

### Example 65. Synthesis of N-(3-((3-(9H-purin-6-yl)pyridin-2-yl)amino)-5-fluoro-4-methylphenyl)-2-(1-cyanocyclopropyl)isonicotinamide (Compound I-62)

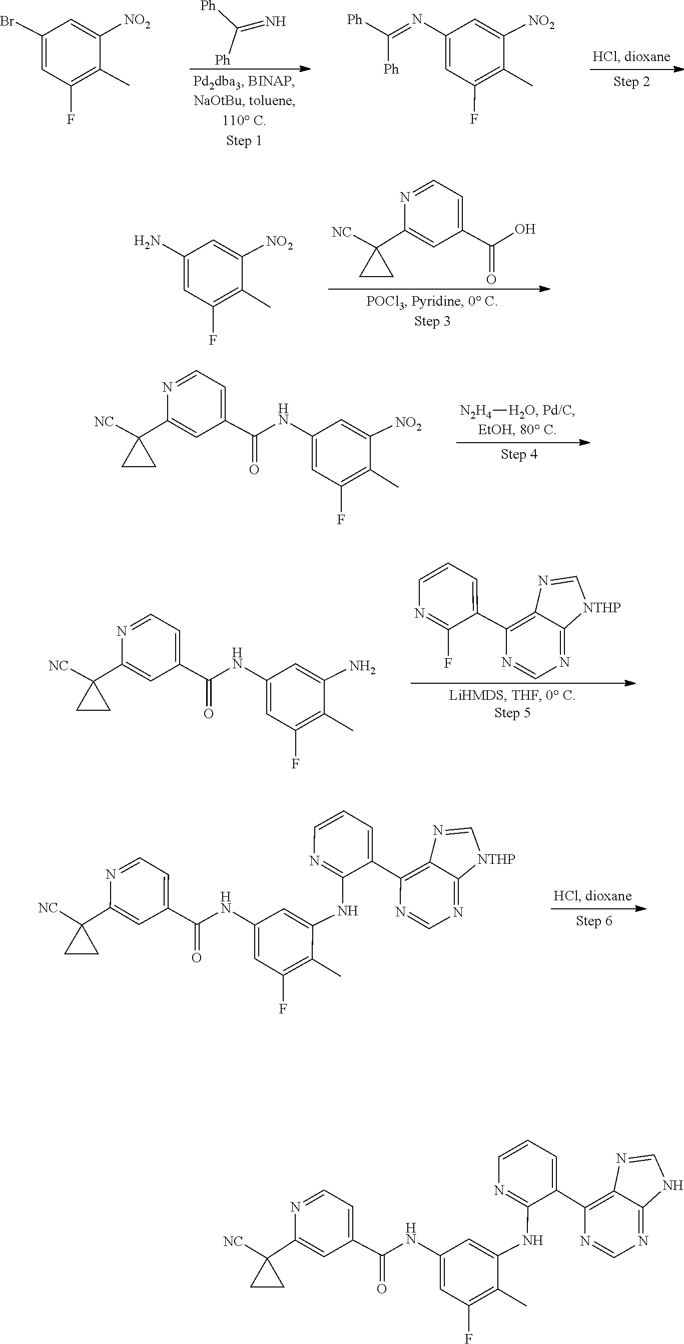

Step 1. A solution of 5-bromo-1-fluoro-2-methyl-3-nitrobenzene (1.0 g, 4.3 mmol), diphenylmethanimine (1.18 g, 6.5 mmol), tri(dibenzylideneacetone)dipalladium (393 mg, 0.43 mmol), (+)-2,2′-bis (diphenylphosphino)-1,1′-binaphthalene (452 mg, 0.86 mmol) and sodium tert-butoxide (1.24 g, 12.9 mmol) in toluene (12 mL) was stirred at 110° C. for 16 h. Water (100 mL) was added and the mixture was extracted with ethyl acetate (50 mL×3). The organic layer was dried over sodium sulfate, filtered, and concentrated. The residue was purified by flash chromatography (ethyl acetate in petroleum ether from 0% to 20%) to give crude N-(diphenylmethylene)-3-fluoro-4-methyl-5-nitroaniline (400 mg) as a white solid. MS (ESI) m/z 335.1 [M+H]⁺

Step 2. A solution of N-(3-fluoro-4-methyl-5-nitrophenyl)-1,1-diphenylmethanimine (400 mg, 1.2 mmol) and hydrogen chloride solution (4 M in dioxane, 2 mL, 8.0 mmol) in dioxane (8 mL) was stirred at 25° C. for 1 h. The mixture was concentrated. The crude residue was purified by silica gel flash chromatography (petroleum ether/ethyl acetate=20/1 to 5/1) to give 3-fluoro-4-methyl-5-nitroaniline (120 mg, 0.71 mmol, 59%) as a yellow solid. MS (ESI) m/z 170.9 [M+H]⁺

Step 3. To an ice-cooled solution of 3-fluoro-4-methyl-5-nitroaniline (110 mg, 0.65 mmol) and 2-(1-cyanocyclopropyl)isonicotinic acid (147 mg, 0.78 mmol) in pyridine (5 mL) was added dropwise phosphorus(V) oxychloride (307 mg, 2.0 mmol). The mixture was stirred at 0° C. for 1 h. The mixture was diluted with ethyl acetate (50 mL) and washed with saturated sodium bicarbonate solution (25 mL) and brine (25 mL). The organic layer was dried over sodium sulfate, filtered and concentrated. The residue was purified by flash chromatography (silica gel, ethyl acetate in petroleum ether from 10% to 40%) to give 2-(1-cyanocyclopropyl)-N-(3-fluoro-4-methyl-5-nitrophenyl)isonicotinamide (110 mg, 0.32 mmol, 49%) as a yellow solid. MS (ESI) m/z 340.9 [M+H]⁺

Step 4. To a solution of 2-(1-cyanocyclopropyl)-N-(3-fluoro-4-methyl-5-nitrophenyl)isonicotinamide (110 mg, 0.32 mmol) and hydrazine hydrate (0.5 mL) in ethanol (15 mL) was added palladium (10% on activated carbon, 50 mg). The mixture was stirred at 80° C. for 1 h. The reaction mixture was cooled to room temperature, filtered, and concentrated. The residue was diluted with ethyl acetate (30 mL) and washed with water (30 mL). The organic phase was washed with brine (30 mL), dried over sodium sulfate, filtered, and concentrated. The residue was purified by flash chromatography (petroleum ether/ethyl acetate=90/100) to afford N-(3-amino-5-fluoro-4-methylphenyl)-2-(1-cyanocyclopropyl)isonicotinamide (90 mg, 0.29 mmol, 91%) as a yellow solid. MS (ESI) m/z 311.0 [M+H]⁺

Step 5. To a solution of N-(3-amino-5-fluoro-4-methylphenyl)-2-(1-cyanocyclopropyl)isonicotinamide (90 mg, 0.29 mmol), 6-(2-fluoropyridin-3-yl)-9- (tetrahydro-2H-pyran-2-yl)-9H-purine (87 mg, 0.29 mmol) in tetrahydrofuran (20 mL) was added lithium hexamethyldisilazide (1 M in tetrahydrofuran, 1.45 mL, 1.45 mmol) at 0° C. slowly. The mixture was stirred at 0° C. for 2 h. The reaction was quenched with saturated ammonium chloride solution (20 mL) at 0° C. and extracted with dichloromethane (50 mL×3). The combined organic layer was washed with brine (50 mL), dried over sodium sulfate, and filtered. The filtrate was concentrated under reduced pressure. The crude residue was purified by flash chromatography (methanol/dichloromethane=12/100) to afford 2-(1-cyanocyclopropyl)-N-(3-fluoro-4-methyl-5-(3-(9-(tetrahydro-2H-pyran-2-yl)-9H-purin-6-yl)pyridin-2-ylamino)phenyl)isonicotinamide (75 mg, 0.13 mmol, 45%) as a yellow solid. MS (ESI) m/z 590.0 [M+H]⁺

Step 6. To a solution of 2-(1-cyanocyclopropyl)-N-(3-fluoro-4-methyl-5-(3-(9-(tetrahydro-2H-pyran-2-yl)-9H-purin-6-yl)pyridin-2-ylamino)phenyl)isonicotinamide (75 mg, 0.13 mmol) in dioxane (3 mL) was added hydrogen chloride solution (4 M in dioxane, 3 mL) at 25° C., and the mixture was stirred at 25° C. for 3 h. The reaction mixture was concentrated. The residue was purified by prep-HPLC (Column: BOSTON pHlex ODS 21.2×250 mm 120 Å, 10 μm, Mobile Phase: A: water (0.05% formic acid) B: acetonitrile) to afford N-(3-((3-(9H-purin-6-yl)pyridin-2-yl)amino)-5-fluoro-4-methylphenyl)-2-(1-cyanocyclopropyl)isonicotinamide (11.9 mg, 0.02 mmol, 15%) as a yellow solid. ¹H NMR (400 MHZ, DMSO-d₆) δ 12.51 (s, 1H), 10.72 (s, 1H), 9.82 (d, J=7.4 Hz, 1H), 9.11 (s, 1H), 8.75 (s, 1H), 8.71 (d, J=5.0 Hz, 1H), 8.52 (s, 1H), 8.38 (dd, J=4.7, 1.8 Hz, 1H), 7.93 (s, 1H), 7.81 (d, J=5.0 Hz, 1H), 7.54 (d, J=11.9 Hz, 1H), 7.11 (dd, J=7.9, 4.8 Hz, 1H), 2.35 (s, 3H), 1.92-1.85 (m, 2H), 1.81-1.75 (m, 2H). MS (ESI) m/z 506.1 [M+H]⁺

### Example 66. Synthesis of N-(3-((3-(9H-purin-6-yl)pyridin-2-yl)amino)-4,5-dimethylphenyl)-4-(trifluoromethyl)picolinamide (Compound I-63)

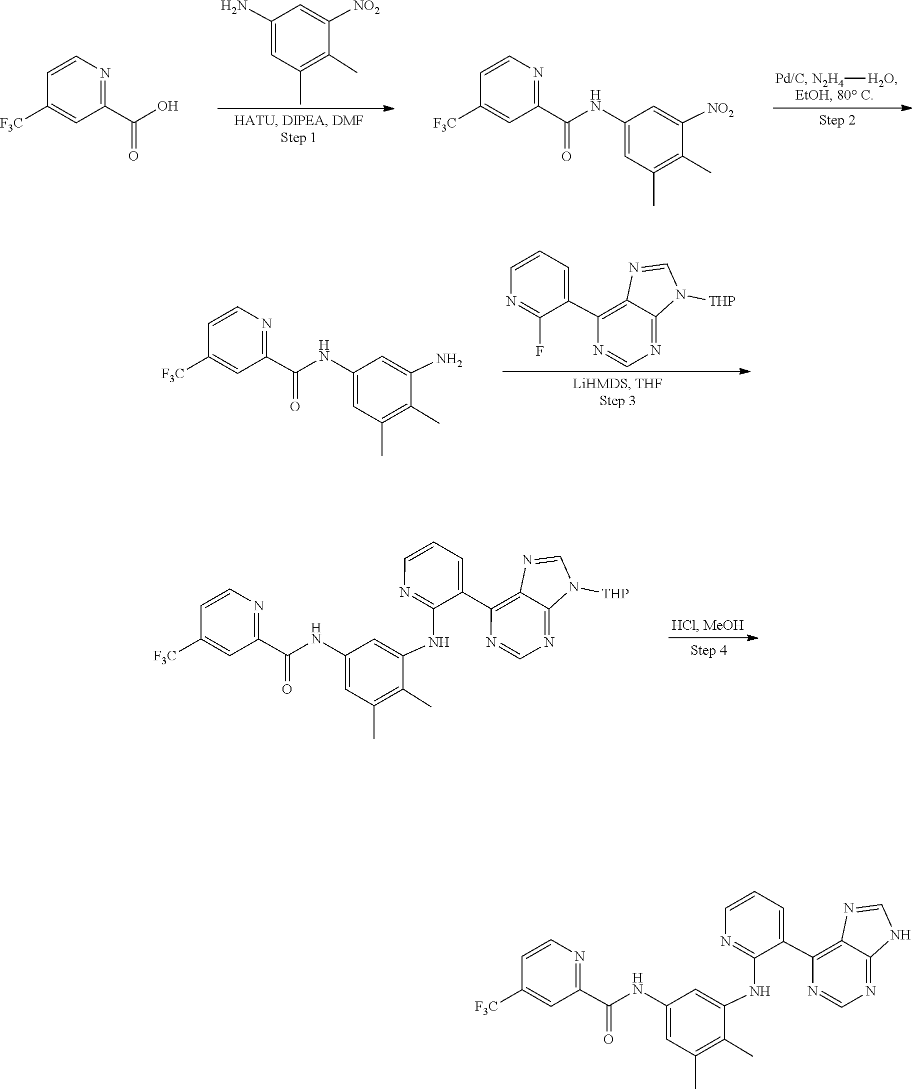

Step 1. A mixture of 4-(trifluoromethyl)picolinic acid (200 mg, 1.05 mmol), 3,4-dimethyl-5-nitroaniline (174 mg, 1.05 mmol), 1-[bis(dimethylamino)methylene]-1H-1,2,3-triazolo[4,5-b]pyridinium 3-oxid hexafluorophosphate (519 mg, 1.37 mmol) and N,N-diisopropylethylamine (271 mg, 2.10 mmol) in N,N-dimethylformamide (10 mL) was stirred at room temperature for 2 h. The reaction was quenched with water (10 mL) and extracted with ethyl acetate (10 mL×3). The combined organic phases were washed with brine, dried over sodium sulfate, filtered, and concentrated to give N-(3,4-dimethyl-5-nitrophenyl)-4-(trifluoromethyl)picolinamide (350 mg, 1.03 mmol, 98%) as a light yellow solid. MS (ESI) m/z 340.0 [M+H]⁺

Step 2. To a solution of N-(3,4-dimethyl-5-nitrophenyl)-4-(trifluoromethyl)picolinamide (350 mg, 1.03 mmol) and hydrazine hydrate (1 mL) in ethanol (10 mL) was added palladium (10% on activated carbon, 35 mg). The mixture was stirred at 80° C. for 1 h. The reaction mixture was filtered directly and concentrated. The residue was triturated with ethanol and dried to give N-(3-amino-4,5-dimethylphenyl)-4-(trifluoromethyl)picolinamide (280 mg, 0.88 mmol, 88%) as a yellow solid. MS (ESI) m/z 310.1 [M+H]⁺

Step 3. To a solution of N-(3-amino-4,5-dimethylphenyl)-4-(trifluoromethyl)picolinamide (100 mg, 0.32 mmol) and 6-(2-fluoropyridin-3-yl)-9-(tetrahydro-2H-pyran-2-yl)-9H-purine (96 mg, 0.32 mmol) in tetrahydrofuran (10 mL) was added lithium hexamethyldisilazide (1.0 mL, 1.6 mmol, 1.6 M in tetrahydrofuran) at 0° C. slowly. The mixture was stirred at room temperature for 1 h. The reaction was quenched with saturated ammonium chloride solution (20 mL) at 0° C. and extracted with ethyl acetate (30 mL×3). The combined organic layer was washed with brine, dried over sodium sulfate, filtered and concentrated. The residue was triturated with ethyl acetate to give N-(3,4-dimethyl-5-((3-(9-(tetrahydro-2H-pyran-2-yl)-9H-purin-6-yl)pyridin-2-yl)amino)phenyl)-4-(trifluoromethyl)picolinamide (150 mg, 0.26 mmol, 80%) as a yellow solid. MS (ESI) m/z 310.1 [M+H]⁺

Step 4. A mixture of N-(3,4-dimethyl-5-((3-(9-(tetrahydro-2H-pyran-2-yl)-9H-purin-6-yl)pyridin-2-yl)amino)phenyl)-4-(trifluoromethyl)picolinamide (150 mg, 0.26 mmol) in hydrogen chloride solution (3 M in methanol, 10 mL) was stirred at room temperature for 3 h. The resulting mixture was concentrated, quenched with ammonia solution in methanol and concentrated again. The residue was triturated with methanol and dried to give N-(3-((3-(9H-purin-6-yl)pyridin-2-yl)amino)-4,5-dimethyl phenyl)-4-(trifluoromethyl)picolinamide (66.2 mg, 0.13 mmol, 51%) as a yellow solid. ¹H NMR (400 MHZ, DMSO-d₆) δ 13.85 (s, 1H), 12.18 (s, 1H), 10.55 (s, 1H), 9.79 (d, J=7.9 Hz, 1H), 9.07 (s, 1H), 9.03 (d, J=5.0 Hz, 1H), 8.71 (s, 1H), 8.42 (s, 1H), 8.34 (s, 1H), 8.30 (d, J=2.9 Hz, 1H), 8.08 (d, J=4.9 Hz, 1H), 7.51 (s, 1H), 6.99 (dd, J=7.8, 4.6 Hz, 1H), 2.33 (s, 3H), 2.30 (s, 3H). MS (ESI) m/z 505.0 [M+H]⁺

### Example 67. Synthesis of N-(3-((3-(9H-purin-6-yl)pyridin-2-yl)amino)-4-methylphenyl)-4-(1-cyanocyclobutyl)picolinamide (Compound I-64)

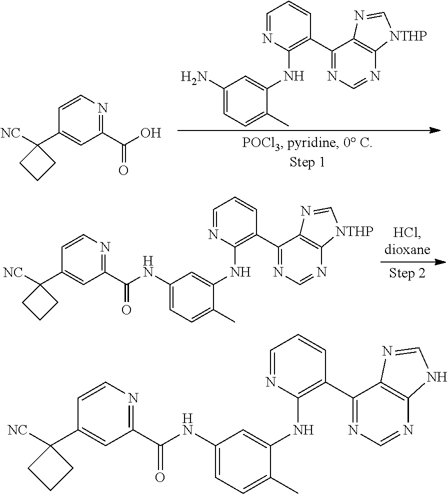

Step 1. To a mixture of 4-(1-cyanocyclobutyl)picolinic acid (55 mg, 0.27 mmol) and 6-methyl-N1-(3-(9-(tetrahydro-2H-pyran-2-yl)-9H-purin-6-yl)pyridin-2-yl)benzene-1,3-diamine (109 mg, 0.27 mmol) in pyridine (3.00 mL) was added dropwise phosphorus(V) oxychloride (124 mg, 0.82 mmol). The mixture was stirred at 0° C. for 1 h under nitrogen. The mixture was extracted with ethyl acetate (50 mL×3). The combined organic layer was washed with brine (50 mL), dried over sodium sulfate, filtered. The filtrate was concentrated under reduced pressure. The residue was purified by flash chromatography (ethyl acetate/petroleum ether=1/1) to give 4-(1-cyanocyclobutyl)-N-(4-methyl-3-((3-(9-(tetrahydro-2H-pyran-2-yl)-9H-purin-6-yl)pyridin-2-yl)amino)phenyl)picolinamide (130 mg, 0.22 mmol, 75%) as a yellow solid. MS (ESI) m/z 585.9 [M+H]⁺

Step 2. To a solution of 4-(1-cyanocyclobutyl)-N-(4-methyl-3-((3-(9-(tetrahydro-2H-pyran-2-yl)-9H-purin-6-yl)pyridin-2-yl)amino)phenyl)picolinamide (130 mg, 0.22 mmol) in methanol (4 mL) was added hydrogen chloride solution (4 M in dioxane, 5 mL) at room temperature, and the mixture was stirred at room temperature for 1 h. After completed, the reaction mixture was concentrated and methanol (4 mL) was added. The pH was adjusted to 8 with ammonia solution (7 N in methanol). The mixture concentrated under reduced pressure and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-4-(1-cyanocyclobutyl)picolinamide (9.4 mg, 0.019 mmol, 9%) as a yellow solid. ¹H NMR (400 MHZ, DMSO-d₆) δ 2.06-2.12 (m, 1H), 2.30-2.37 (m, 1H), 2.43 (s, 3H), 2.73 (q, J=8.8 Hz, 2H), 2.81 (d, J=8.0 Hz, 2H), 7.06 (q, J=4.4 Hz, 1H), 7.27 (d, J=8.4 Hz, 1H), 7.57 (d, J=7.6 Hz, 1H), 7.84 (s, 1H), 8.21 (s, 1H), 8.35 (d, J=3.6 Hz, 1H), 8.75 (d, J=13.6 Hz, 3H), 9.09 (s, 1H), 9.80 (d, J=3.2 Hz, 1H), 10.56 (s, 1H), 12.37 (s, 1H). MS (ESI) m/z 502.1 [M+H]⁺

### Example 68. Synthesis of N-(3-((3-(9H-purin-6-yl)pyridin-2-yl)amino)-4-methylphenyl)-3-methyl-4-(trifluoromethyl)picolinamide (Compound I-65)

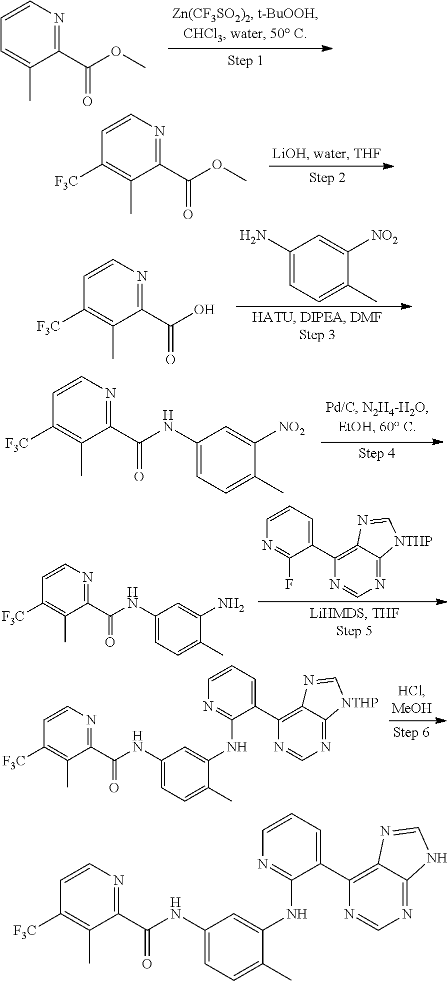

Step 1. To a solution of methyl 3-methylpicolinate (200 mg, 1.32 mmol) in chloroform (10 mL) and water (3 mL) was added zinc trifluoromethanesulfinate (877 mg, 2.64 mmol) and tert-butyl hydroperoxide (70% in water, 506 mg, 3.94 mmol). The mixture was stirred at 50° C. for 16 h. The resulting mixture was quenched with saturated sodium bicarbonate solution and extracted with ethyl acetate (15 mL×3). The combined organic phases were washed with brine, dried over sodium sulfate, filtered, concentrated and purified by column chromatography (10% ethyl acetate in petroleum ether) to give methyl 3-methyl-4-(trifluoromethyl)picolinate (100 mg, 0.46 mmol, 35%) as a colorless oil. MS (ESI) m/z 220.1 [M+H]⁺

Step 2. A mixture of methyl 3-methyl-4-(trifluoromethyl)picolinate (100 mg, 0.46 mmol) and lithium hydroxide monohydrate (39 mg, 0.92 mmol) in tetrahydrofuran (10 mL) and water (1 mL) was stirred at room temperature for 3 h. The resulting mixture was diluted with water (10 mL) and acidified with hydrochloric acid to pH=5, then extracted with ethyl acetate (10 mL×3). The combined organic phases were washed with brine, dried over sodium sulfate, filtered and concentrated to give 3-methyl-4-(trifluoromethyl)picolinic acid (60 mg, 0.29 mmol, 64%) as a white solid. MS (ESI) m/z 206.1 [M+H]⁺

Step 3. A mixture of 3-methyl-4-(trifluoromethyl)picolinic acid (60 mg, 0.29 mmol), 4-methyl-3-nitroaniline (44 mg, 0.29 mmol), 1-[bis(dimethylamino)methylene]-1H-1,2,3-triazolo[4,5-b]pyridinium 3-oxid hexafluorophosphate (165 mg, 0.44 mmol) and N,N-diisopropylethylamine (75 mg, 0.58 mmol) in N,N-dimethylformamide (5 mL) was stirred at room temperature for 2 h. The mixture was quenched with water (10 mL) and extracted with ethyl acetate (10 mL×3). The combined organic phases were washed with brine, dried over sodium sulfate, filtered and concentrated to give 3-methyl-N-(4-methyl-3-nitrophenyl)-4-(trifluoromethyl)picolinamide (80 mg, 0.24 mmol, 81%) as a light yellow solid. MS (ESI) m/z 340.3 [M+H]⁺

Step 4. To a solution of 3-methyl-N-(4-methyl-3-nitrophenyl)-4-(trifluoromethyl)picolinamide (80 mg, 0.24 mmol) and hydrazine hydrate (1 mL) in ethanol (10 mL) was added palladium (10% on activated carbon, 10 mg). The mixture was stirred at 80° C. for 1 h. The reaction mixture was filtered and the filtrate was concentrated. The residue was triturated with ethanol and dried to give N-(3-amino-4,5-dimethylphenyl)-4-(trifluoromethyl)picolinamide (60 mg, 0.19 mmol, 81%) as a yellow solid. MS (ESI) m/z 310.2 [M+H]⁺

Step 5. To a solution of N-(3-amino-4,5-dimethylphenyl)-4-(trifluoromethyl)picolinamide (60 mg, 0.19 mmol) and 6-(2-fluoropyridin-3-yl)-9-(tetrahydro-2H-pyran-2-yl)-9H-purine (57 mg, 0.19 mmol) in tetrahydrofuran (10 mL) was added lithium hexamethyldisilazide (0.59 mL, 0.95 mmol, 1.6 M in tetrahydrofuran) at 0° C. slowly. The mixture was stirred at room temperature for 1 h. The reaction was quenched with saturated ammonium chloride solution (20 mL) at 0° C. and extracted with ethyl acetate (30 mL×3). The combined organic layer was washed with brine, dried over sodium sulfate, filtered and concentrated. The residue was triturated with ethyl acetate to give 3-methyl-N-(4-methyl-3-((3-(9-(tetrahydro-2H-pyran-2-yl)-9H-purin-6-yl)pyridin-2-yl)amino)phenyl)-4-(trifluoromethyl)picolinamide (60 mg, 0.10 mmol, 54%) as a yellow solid. MS (ESI) m/z 589.2 [M+H]⁺

Step 6. A mixture of 3-methyl-N-(4-methyl-3-((3-(9-(tetrahydro-2H-pyran-2-yl)-9H-purin-6-yl)pyridin-2-yl)amino)phenyl)-4-(trifluoromethyl)picolinamide (60 mg, 0.10 mmol) in hydrogen chloride solution (3 M in methanol, 10 mL) was stirred at 20° C. for 3 h. The resulting mixture was concentrated, quenched with ammonia solution in methanol and concentrated again. The residue was triturated with methanol and dried to give N-(3-((3-(9H-purin-6-yl)pyridin-2-yl)amino)-4-methylphenyl)-3-methyl-4-(trifluoromethyl)picolinamide (37.7 mg, 0.075 mmol, 75%) as a yellow solid. ¹H NMR (400 MHZ, DMSO-d₆) δ 12.32 (s, 1H), 10.56 (s, 1H), 9.78 (d, J=7.1 Hz, 1H), 9.08 (s, 1H), 8.84-8.58 (m, 3H), 8.34 (dd, J=4.6, 1.9 Hz, 1H), 7.86 (d, J=5.0 Hz, 1H), 7.51 (dd, J=8.1, 2.0 Hz, 1H), 7.25 (d, J=8.3 Hz, 1H), 7.03 (dd, J=7.8, 4.7 Hz, 1H), 2.54 (s, 3H), 2.44 (s, 3H). MS (ESI) m/z 505.1 [M+H]⁺

### Example 69. Synthesis of N-(3-((3-(9H-purin-6-yl)pyridin-2-yl)amino)-4-methylphenyl)-2-(1-cyanocyclobutyl)isonicotinamide (Compound I-66)

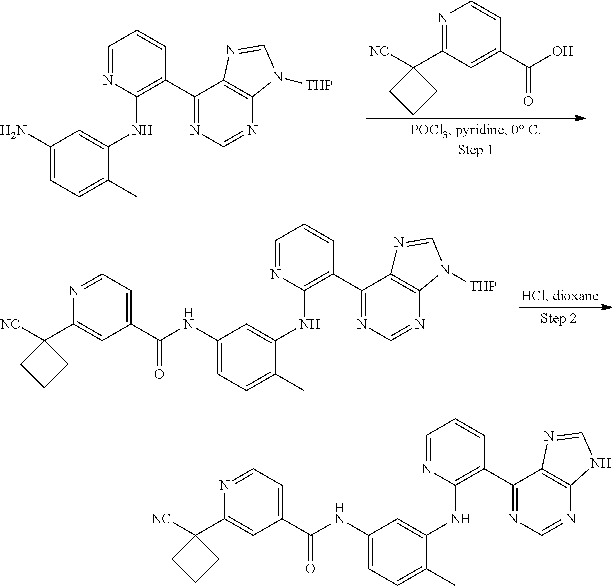

Step 1. phosphorus(V) oxychloride (66 mg, 0.441 mmol) was added to a mixture of 2-(1-cyanocyclobutyl)isonicotinic acid (30 mg, 0.147 mmol) and 6-methyl-N1-(3-(9-(tetrahydro-2H-pyran-2-yl)-9H-purin-6-yl)pyridin-2-yl)benzene-1,3-diamine (60 mg, 0.147 mmol) in pyridine (2 mL) at 0° C. The reaction mixture was stirred at 0° C. for 2 h under nitrogen atmosphere. The reaction mixture was extracted with ethyl acetate (50 mL×3) and water (30 mL). The organic layer was dried over a sodium sulfate, concentrated in vacuo and the residue was purified by silica gel column chromatography (petroleum ether/ethyl acetate=10/1 to 1/1) to afford 2-(1-cyanocyclobutyl)-N-(4-methyl-3-((3-(9-(tetrahydro-2H-pyran-2-yl)-9H-purin-6-yl)pyridin-2-yl)amino)phenyl)isonicotinamide (60 mg, 0.10 mmol, 69%) as a yellow solid. MS (ESI) m/z 586.2 [M+H]⁺

Step 2. To a solution of 2-(1-cyanocyclobutyl)-N-(4-methyl-3-((3-(9-(tetrahydro-2H-pyran-2-yl)-9H-purin-6-yl)pyridin-2-yl)amino)phenyl)isonicotinamide (60 mg, 0.10 mmol) in 1,4-dioxane was added hydrogen chloride solution (6 M in dioxane, 10 mL). The reaction mixture was stirred at room temperature for 0.5 h. The reaction mixture was concentrated under reduced pressure and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-2-(1-cyanocyclobutyl)isonicotinamide (10 mg, 0.02 mmol, 20%) as a yellow solid. ¹HNMR (400 MHz, DMSO-d₆) δ=13.87 (s, 1H), 12.34 (s, 1H), 10.54 (s, 1H), 9.82 (s, 1H), 9.09 (s, 1H), 8.83-8.82 (d, J=4.8 Hz), 8.72 (s, 1H), 8.70 (s, 1H), 8.37-8.35 (m, 1H), 8.03 (s, 1H), 7.90-7.89 (d, J=4.4 Hz), 7.47-7.45 (d, J=8.0 Hz), 7.27-7.25 (d, J=8.4 Hz), 7.06-7.03 (m, 1H), 2.89-2.73 (m, 4H), 2.45 (s, 3H), 2.35-2.28 (m, 1H), 2.11-2.08 (m, 1H). MS (ESI) m/z 502.2 [M+H]⁺

### Example 70. Synthesis N-(4-methyl-3-((6-methyl-3-(9H-purin-6-yl)pyridin-2-yl)amino)phenyl)-4-(trifluoromethyl)picolinamide (Compound I-67)

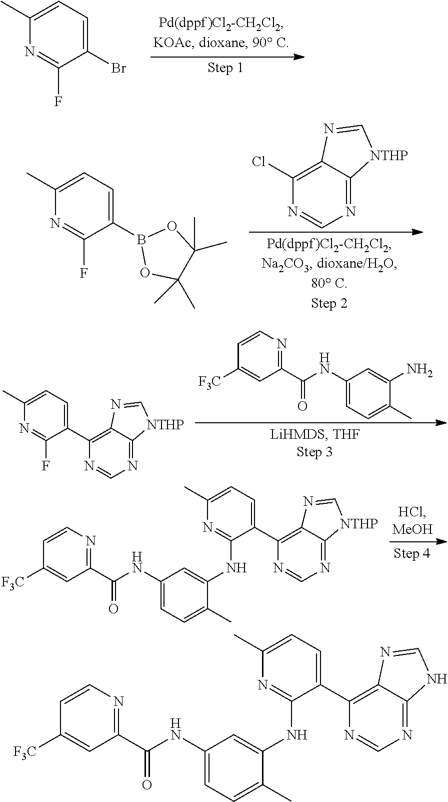

Step 1. A solution of 3-bromo-2-fluoro-6-methylpyridine (380 mg, 2.0 mmol), 4,4,4′,4′,5,5,5′,5′-octamethyl-2,2′-bi(1,3,2-dioxaborolane) (610 mg, 2.4 mmol), 1,1′-bis(diphenylphosphino)ferrocene palladium(II)dichloride dichloromethane complex (163 mg, 0.2 mmol) and potassium acetate (588 mg, 6.0 mmol) in dioxane (15 mL) was stirred at 90° C. for 16 h under argon. The mixture was used directly in the next step. MS (ESI) m/z 238.1 [M+H]⁺

Step 2. To a solution of 2-fluoro-6-methyl-3-(4,4,5,5-tetramethyl-1,3,2-dioxaborolan-2-yl)pyridine (The previous reaction solution) (474 mg, 2.0 mmol) in dioxane (15 mL) and water (3 ml) was added 6-chloro-9-(tetrahydro-2H-pyran-2-yl)-9H-purine (310 mg, 1.3 mmol), sodium carbonate (636 mg, 6.0 mmol), 1,1′-bis(diphenylphosphino)ferrocene-palladium(II)dichloride dichloromethane complex (163 mg, 0.2 mmol). The mixture was stirred at 80° C. under argon for 16 h. The reaction mixture was cooled. The organic layer was concentrated and purified by flash chromatography (silica gel, ethyl acetate in petroleum ether from 30% to 90%) to give 6-(2-fluoro-6-methylpyridin-3-yl)-9-(tetrahydro-2H-pyran-2-yl)-9H-purine (280 mg, 0.89 mmol, 68%) as a yellow solid. MS (ESI) m/z 314.0 [M+H]⁺

Step 3. To a solution of N-(3-amino-4-methylphenyl)-4-(trifluoromethyl)picolinamide (280 mg, 0.95 mmol), 6-(2-fluoro-6-methylpyridin-3-yl)-9-(tetrahydro-2H-pyran-2-yl)-9H-purine (298 mg, 0.95 mmol) in tetrahydrofuran (20 mL) was slowly added lithium hexamethyldisilazide (4.8 mL, 7.6 mmol, 1.6 M in tetrahydrofuran) at 0° C. The mixture was stirred at 0° C. for 1 h. The reaction was quenched with saturated ammonium chloride solution (20 mL) at 0° C. and extracted with dichloromethane (50 mL×3). The combined organic layer was washed with brine (50 mL), dried over sodium sulfate and filtered. The filtrate was concentrated under reduced pressure. The crude residue was purified by flash chromatography (methanol/dichloromethane=5:100) to give N-(4-methyl-3-(6-methyl-3-(9-(tetrahydro-2H-pyran-2-yl)-9H-purin-6-yl)pyridin-2-ylamino)phenyl)-4-(trifluoromethyl)picolinamide (160 mg, 0.27 mmol, 28%) as a yellow solid. MS (ESI) m/z 589.2 [M+H]⁺

Step 4. To a solution of N-(4-methyl-3-(6-methyl-3-(9-(tetrahydro-2H-pyran-2-yl)-9H-purin-6-yl)pyridin-2-ylamino)phenyl)-4-(trifluoromethyl)picolinamide (160 mg, 0.27 mmol) in methanol (10 mL) was added hydrogen chloride solution (3M in methanol, 5 mL) at 25° C., and stirred for 1 h. After completed, the reaction mixture was concentrated. The residue was diluted with methanol (10 mL) and the pH was adjusted to 8 by adding ammonia (7 N in methanol) solution. The mixture concentrated under reduced pressure. The residue was triturated with methanol (10 mL) and filtered. The solid was suspended in water (50 mL) and lyophilized to afford N-(4-methyl-3-((6-methyl-3-(9H-purin-6-yl)pyridin-2-yl)amino)phenyl)-4-(trifluoromethyl)picolinamide (92.5 mg, 0.18 mmol, 67%) as a yellow solid. ¹H NMR (400 MHZ, DMSO-d₆) δ 13.81 (s, 1H), 12.41 (s, 1H), 10.61 (s, 1H), 9.74 (s, 1H), 9.21 (d, J=2.0 Hz, 1H), 9.08-8.99 (m, 2H), 8.69 (s, 1H), 8.35 (s, 1H), 8.09 (d, J=3.9 Hz, 1H), 7.41 (dd, J=8.1, 2.1 Hz, 1H), 7.24 (d, J=8.3 Hz, 1H), 6.92 (d, J=8.1 Hz, 1H), 2.51 (s, 3H), 2.46 (s, 3H). MS (ESI) m/z 505.1 [M+H]⁺

### Example 71. Synthesis of N-(3-((3-(9H-purin-6-yl)pyridin-2-yl)amino)-4-ethylphenyl)-4-(trifluoromethyl)picolinamide (Compound I-68)

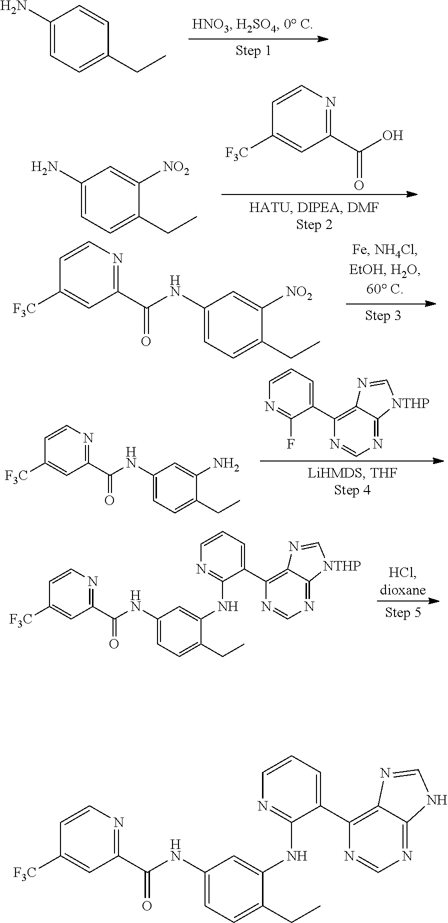

Step 1. To a solution of 4-ethylaniline (3.0 g, 24.76 mmol) in concentrated sulfuric acid (20 mL) was added a solution of fuming nitric acid (1.2 mL) in concentrated sulfuric acid (3 mL) at 0° C. The reaction mixture was stirred at 0° C. for 1 h. The reaction mixture was poured into ice water (100 mL) slowly. The precipitate was filtered and washed with water. The solid was suspended with water and neutralized with ammonium hydroxide. The mixture was filtered and the solid was dried under reduced pressure to give 4-ethyl-3-nitroaniline (2.8 g, 16.85 mmol, 68%) as a yellow solid. ¹H NMR (400 MHZ, DMSO-d₆) δ 7.23-7.21 (m, 2H), 6.89-6.96 (m, 1H), 2.67 (q, J=7.6 Hz, 15.2 Hz, 2H), 1.13 (t, J=7.2 Hz, 3H).

Step 2. To a mixture of 4-(trifluoromethyl)picolinic acid (230 mg, 1.20 mmol) in N,N-dimethylformamide (10 mL) was added 1-[bis(dimethylamino)methylene]-1H-1,2,3-triazolo[4,5-b]pyridinium 3-oxid hexafluorophosphate (549 mg, 1.44 mmol) and N,N-diisopropylethylamine (232 mg, 1.81 mmol) at room temperature. After stirring for 5 min, 4-ethyl-3-nitroaniline (200 mg, 1.20 mmol) was added. The mixture was allowed to stir at room temperature for another 3 h. Water (50 mL) was added to the reaction mixture, which was then extracted with ethyl acetate (50 mL×3). The organic phases were concentrated and purified by flash chromatography on silica gel (petroleum ether ethyl acetate=5/1) to give N-(4-ethyl-3-nitrophenyl)-4-(trifluoromethyl)picolinamide (250 mg, 0.74 mmol, 61%) as a yellow solid.

Step 3. To a solution of N-(4-ethyl-3-nitrophenyl)-4-(trifluoromethyl)picolinamide (250 mg, 0.74 mmol) in ethanol (3 mL) and water (3 mL) was added ammonium chloride (198 mg, 3.70 mmol) and iron powder (207 mg, 3.70 mmol). The reaction mixture was stirred at 60° C. for 1 h. The reaction mixture was filtered and the filtrate was concentrated and diluted with ethyl acetate (10 mL). The organic phase was washed with brine (20 mL), dried over sodium sulfate and filtered. The filtrate was concentrated to give N-(3-amino-4-ethylphenyl)-4-(trifluoromethyl)picolinamide (220 mg, 0.71 mmol, 96%) as a yellow solid. MS (ESI) m/z 310.0 [M+H]⁺

Step 4. To a solution of N-(3-amino-4-ethylphenyl)-4-(trifluoromethyl)picolinamide (180 mg, 0.58 mmol) and 6-(2-fluoropyridin-3-yl)-9-(tetrahydro-2H-pyran-2-yl)-9H-purine (209 mg, 0.70 mmol) in tetrahydrofuran (5 mL) was added lithium hexamethyldisilazide (1.8 mL, 2.91 mmol, 1.6 M in tetrahydrofuran) at 0° C. under a nitrogen atmosphere. The reaction mixture was stirred at room temperature for 16 h and poured into water (10 mL). The mixture was extracted with ethyl acetate (10 mL×3). The organic layers were washed with water (20 mL×3) and brine (20 mL), dried over sodium sulfate, filtered and concentrated. The residue was purified by flash chromatography (petroleum ether/ethyl acetate=1/1) to give N-(4-ethyl-3-((3-(9-(tetrahydro-2H-pyran-2-yl)-9H-purin-6-yl)pyridin-2-yl)amino)phenyl)-4-(trifluoromethyl)picolinamide (180 mg, 0.31 mmol, 53%) as a yellow solid. MS (ESI) m/z 589.2 [M+H]⁺

Step 5. A solution of N-(4-ethyl-3-((3-(9-(tetrahydro-2H-pyran-2-yl)-9H-purin-6-yl)pyridin-2-yl)amino)phenyl)-4-(trifluoromethyl)picolinamide (100 mg, 0.17 mmol) in hydrogen chloride solution (4 M in dioxane, 2 mL) was stirred at room temperature for 0.5 h. The solvent was removed under reduced pressure and the residue was treated with ammonia (1 mL, 8 M in methanol). The resulting mixture was filtered and the solid was collected. The solid was dried under reduced pressure to give N-(3-((3-(9H-purin-6-yl)pyridin-2-yl)amino)-4-ethylphenyl)-4-(trifluoromethyl)picolinamide (76.4 mg, 0.15 mmol, 89%) as a yellow solid. ¹H NMR (400 MHZ, DMSO-d₆) δ 13.86 (s, 1H), 12.26 (s, 1H), 10.65 (s, 1H), 9.80 (s, 1H), 9.06-9.03 (m, 2H), 8.74-8.73 (m, 2H), 8.36-8.33 (m, 2H), 8.09 (d, J=6.4 Hz, 1H), 7.60-7.57 (m, 1H), 7.26 (d, J=8.4 Hz, 1H), 7.04-7.01 (m, 1H), 2.82 (q, J=7.2 Hz, 14.8 Hz, 2H), 1.29 (t, J=7.6 Hz, 3H). MS (ESI) m/z 505.1 [M+H]⁺

### Example 72. Synthesis of N-(3-((3-(9H-purin-6-yl)pyridin-2-yl)amino)-4-methylphenyl)-4-chloro-3-(trifluoromethoxy)benzamide (Compound I-69)

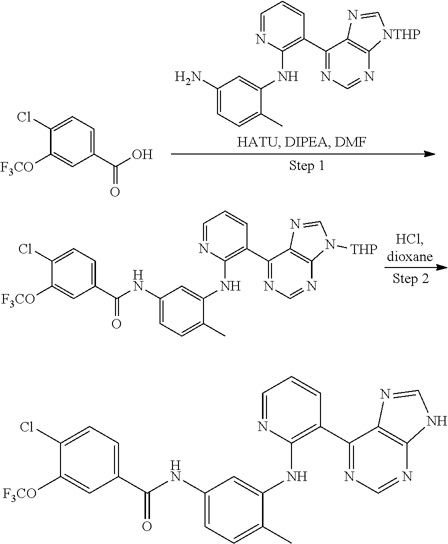

Step 1. A mixture of 4-chloro-3-(trifluoromethoxy)benzoic acid (39 mg, 0.16 mmol), 6-methyl-N1-(3-(9-(tetrahydro-2H-pyran-2-yl)-9H-purin-6-yl)pyridin-2-yl)benzene-1,3-diamine (60 mg, 0.15 mmol), 1-[bis(dimethylamino)methylene]-1H-1,2,3-triazolo[4,5-b]pyridinium 3-oxid hexafluorophosphate (62.5 mg, 0.16 mmol) and N,N-diisopropylethylamine (38 mg, 0.3 mmol) in N,N-dimethylformamide (6 mL). The reaction mixture was stirred at 25° C. for 2 h. The reaction mixture was extracted with ethyl acetate (10 mL×3). The combined organic layers were washed with brine (20 mL), dried over sodium sulfate, filtered and concentrated. The residue was purified by flash chromatography (silica gel, petroleum ether/ethyl acetate=1/1) to give 4-chloro-N-(4-methyl-3-((3-(9-(tetrahydro-2H-pyran-2-yl)-9H-purin-6-yl)pyridin-2-yl)amino)phenyl)-3-(trifluoromethoxy)benzamide (80 mg, 0.128 mmol, 85%) as a yellow solid. MS (ESI) m/z 624.1 [M+H]⁺

Step 2. A solution of 4-chloro-N-(4-methyl-3-((3-(9-(tetrahydro-2H-pyran-2-yl)-9H-purin-6-yl)pyridin-2-yl)amino)phenyl)-3-(trifluoromethoxy)benzamide (80 mg, 0.128 mmol) in hydrogen chloride solution (4 M in dioxane, 6 mL) was stirred at 25° C. for 1 h. The solvent was removed under reduced pressure and the residue was treated with ammonia (5 mL, 7 M in methanol). The solvent was removed once more under reduced pressure and concentrated. The residue was washed with methanol (20 mL) and water (5 mL) to give N-(3-((3-(9H-purin-6-yl)pyridin-2-yl)amino)-4-methylphenyl)-4-chloro-3-(trifluoromethoxy)benzamide (48.5 mg, 0.09 mmol, 70%) as a yellow solid. ¹H NMR (400 MHZ, DMSO-d₆) δ 12.33 (s, 1H), 10.42 (s, 1H), 9.79 (d, J=6.9 Hz, 1H), 9.34-8.91 (m, 1H), 8.70 (s, 1H), 8.67 (d, J=1.6 Hz, 1H), 8.36 (d, J=2.8 Hz, 1H), 8.15-8.04 (m, 2H), 7.89 (d, J=8.3 Hz, 1H), 7.45 (d, J=6.5 Hz, 1H), 7.25 (d, J=8.2 Hz, 1H), 7.04 (dd, J=7.8, 4.7 Hz, 1H), 2.45 (s, 3H). MS (ESI) m/z 540.0 [M+H]⁺

### Example 73. Synthesis of N-(3-((3-(9H-purin-6-yl)pyridin-2-yl)amino)-4-methylphenyl)-5-cyano-6-(trifluoromethyl)nicotinamide (Compound I-70)

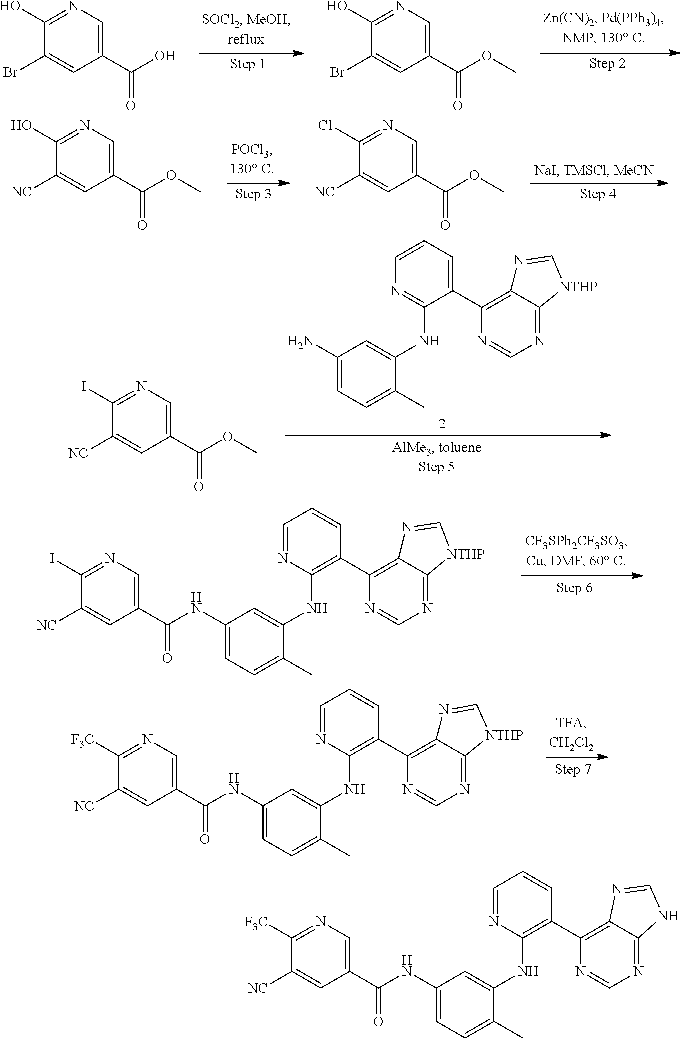

Step 1. To an ice-cold stirred solution of 5-bromo-6-hydroxy nicotinic acid (2.03 g, 9.31 mmol) in anhydrous methanol (40 mL) was added thionyl chloride (1.01 mL, 13.9 mmol) dropwise. The reaction mixture was stirred for 15 min and then was heated to reflux. Upon completion (1 h) the solvent was removed under reduced pressure, and the residue was taken up in ethyl acetate (150 mL). The mixture was washed with water (2×150 mL), and brine (150 mL), and dried over sodium sulfate. The solids were filtered, and solvent was removed under reduced pressure to afford methyl 5-bromo-6-hydroxynicotinate (1.8 g, 7.72 mmol, 83%) as a yellow solid. MS (ESI) m/z 234.0 [M+H]⁺

Step 2. A mixture of tetrakis(triphenylphosphine)palladium (700 mg, 0.6 mmol), methyl 5-bromo-6-hydroxy nicotinate (1.5 g, 6.0 mmol) and zinc cyanide (1.7 g, 12.0 mmol) in 1-methyl-2-pyrrolidinone (30 mL) was stirred at 130° C. overnight under nitrogen. The mixture was purified by reversed phase column chromatography eluting with water/acetontrile (2/1) to give methyl 5-cyano-6-hydroxynicotinate (865 mg, 4.86 mmol, 81%) as a white solid. MS (ESI) m/z 179.1 [M+H]⁺

Step 3. A mixture of methyl 5-cyano-6-hydroxynicotinate (850 mg, 4.775 mmol) and several drops of N,N-diisopropylethylamine in phosphorus(V) oxychloride (30 mL) was stirred at 130° C. overnight. The mixture concentrated to dryness in vacuo and purified by flash chromatography (silica gel, ethyl acetate in petroleum ether 50%) to afford methyl 6-chloro-5-cyanonicotinate (750 mg, 3.826 mmol, 80%) as a white solid. MS (ESI) m/z 197.7 [M+H]⁺

Step 4. Chlorotrimethylsilane (0.17 mL) was added to a solution of methyl 6-chloro-5-cyanonicotinate (240 mg, 0.5 mmol) and sodium iodine (1.5 g, 10 mmol) in acetonitrile (15 mL). The resulted mixture was stirred at room temperature for 1.5 h. The mixture was diluted with water (20 mL) and extracted with ethyl acetate (20 mL×3). The organic layers were combined, dried over sodium sulfate and concentrated under reduced pressure. The residue was purified by flash chromatography (silica gel, petroleum ether/ethyl acetate=3/7) to give methyl 5-cyano-6-iodonicotinate (190 mg, 0.66 mmol, 54%) as a white solid. MS (ESI) m/z 288.7 [M+H]⁺

Step 5. To a solution of 6-methyl-N1-(3-(9-(tetrahydro-2H-pyran-2-yl)-9H-purin-6-yl)pyridin-2-yl)benzene-1,3-diamine (196 mg, 0.49 mmol) in toluene (16 mL) was added trimethylaluminum (2.0 mL, 2.0 mmol) slowly at 0° C. under argon. The mixture was stirred at room temperature for 2 h. Then methyl 5-cyano-6-iodonicotinate (60 mg, 0.208 mmol) in toluene (7.0 mL) was added to the mixture, the resulting mixture was heated to 100° C. and stirred for 3 h. The mixture was cooled to 25° C. and water (20 mL) was added. The mixture was extracted with ethyl acetate (20 mL×3). The combined organic phases were dried over sodium sulfate and concentrated. The crude residue was purified by flash chromatography (silica gel, petroleum ether/ethyl acetate=3/7 to 100% ethyl acetate) to give 5-cyano-6-iodo-N-(4-methyl-3-((3-(9-(tetrahydro-2H-pyran-2-yl)-9H-purin-6-yl)pyridin-2-yl)amino)phenyl)nicotinamide (400 mg, 0.608 mmol, 88%). MS (ESI) m/z 658.7 [M+H]⁺

Step 6. A mixture of 5-cyano-6-iodo-N-(4-methyl-3-((3-(9-(tetrahydro-2H-pyran-2-yl)-9H-purin-6-yl)pyridin-2-yl)amino)phenyl)nicotinamide (330 mg, 0.5 mmol), diphenyl(trifluoromethyl)sulfonium trifluoromethanesulfonate (400 mg, 1.0 mmol) and copper (100 mg, 1.5 mmol) in N,N-dimethylformamide (10 mL) was stirred at 60° C. for 16 h under nitrogen. After cooling to room temperature, the mixture was washed with water (20 mL) and extracted with ethyl acetate (5 mL×3). The organic phase were dried over sodium sulfate and concentrated. The crude residue was purified by flash chromatography (petroleum ether/ethyl acetate=3/7) to give 5-cyano-N-(4-methyl-3-((3-(9-(tetrahydro-2H-pyran-2-yl)-9H-purin-6-yl)pyridin-2-yl)amino)phenyl)-6-(trifluoromethyl)nicotinamide (180 mg, 0.3 mmol, 60%) as a yellow solid. MS (ESI) m/z 600.7 [M+H]⁺

Step 7. A solution of 5-cyano-N-(4-methyl-3-((3-(9-(tetrahydro-2H-pyran-2-yl)-9H-purin-6-yl)pyridin-2-yl)amino)phenyl)-6-(trifluoromethyl)nicotinamide (100 mg, 0.167 mmol) in trifluoroacetic acid/dichloromethane (5 mL/5 mL) was stirred at room temperature for 1 h. The mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-5-cyano-6-(trifluoromethyl)nicotinamide (50.8 mg, 0.0848 mmol, 51%) as a yellow solid.

¹H NMR (400 MHZ, DMSO-d₆) δ 13.89 (s, 1H), 12.42 (s, 1H), 10.71 (s, 1H), 9.85 (d, J=7.6 Hz, 1H), 9.44 (s, 1H), 9.16 (s, 1H), 9.10 (s, 1H), 8.76 (s, 1H), 8.74 (s, 1H), 8.38 (m, 1H), 7.51 (d, J=6.4 Hz, 1H), 7.29 (d, J=7.6 Hz, 1H), 7.07 (m, 1H), 2.45 (s, 3H). MS (ESI) m/z 516.7 [M+H]⁺

### Example 74. Synthesis of N-(3-((3-(9H-purin-6-yl)pyridin-2-yl)amino)-4-methylphenyl)-4-cyano-5-methylpicolinamide (Compound I-71)

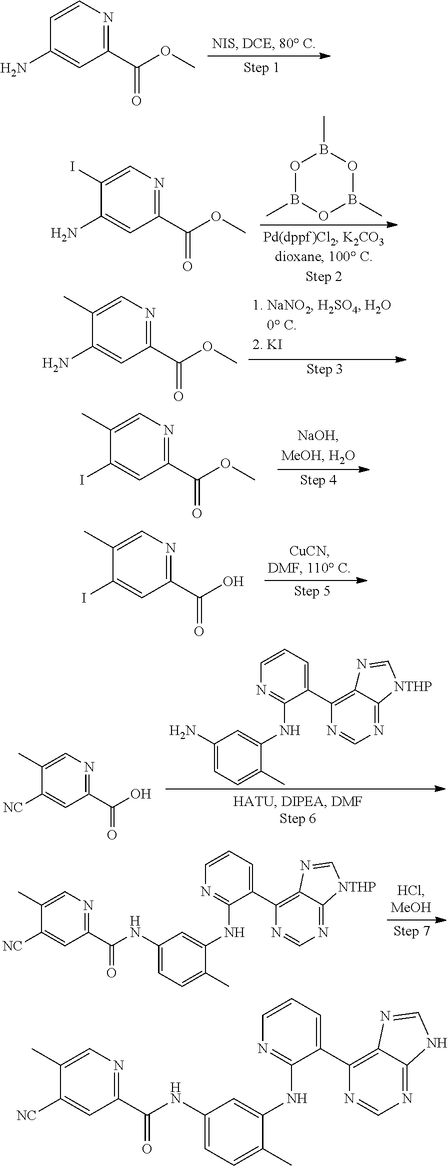

Step 1. A mixture of methyl 4-aminopicolinate (1520 mg, 10 mmol) and N-iodosuccinimide (2700 mg, 12 mmol) in dichloroethane (50 mL) was stirred at 80° C. for 16 h. The mixture was concentrated in vacuo. The residue was diluted with water (100 mL) and extracted with ethyl acetate (3×100 mL). The combined organic phases were concentrated in vacuo. The residue was purified with flash chromatography (silica gel, ethyl acetate/petroleum ether=0% to 50%) to afford methyl 4-amino-5-iodopicolinate (1500 mg, 5.39 mmol, 54%) as a white solid. MS (ESI) m/z 279 [M+H]⁺

Step 2. A mixture of methyl 4-amino-5-iodopicolinate (1390 mg, 5 mmol), trimethylboroxine (1260 mg, 10 mmol), [1,1′-bis(diphenylphosphino)ferrocene]dichloropalladium(II) (365 mg, 0.5 mmol) and potassium carbonate (2070 mg, 15 mmol) in dioxane (20 mL) was stirred at 100° C. for 4 h. The mixture was concentrated in vacuo. The residue was diluted with water (100 mL) and extracted with ethyl acetate (3×100 mL). The combined organic phases were concentrated in vacuo. The residue was purified by flash chromatography (silica gel, ethyl acetate/petroleum ether=0% to 50%) to afford methyl 4-amino-5-methylpicolinate (600 mg, 3.6 mmol, 72%) as a white solid. MS (ESI) m/z 167 [M+H]⁺

Step 3. A mixture of methyl 4-amino-5-methylpicolinate (664 mg, 4 mmol) and sodium nitrite (303 mg, 4.4 mmol) in 50% sulfuric acid aqueous solution (20 mL) was stirred at 0° C. for 0.5 h. Then a solution of potassium iodide (3320 mg, 20 mmol) in water (10 mL) was added and the reaction mixture was stirred at room temperature for 4 h. The mixture was concentrated in vacuo. The residue was diluted with water (100 mL) and extracted with ethyl acetate (3×100 mL). The combined organic phases were concentrated in vacuo. The residue was purified by flash chromatography (silica gel, ethyl acetate/petroleum ether=0% to 50%) to afford methyl 4-iodo-5-methylpicolinate (700 mg, 2.53 mmol, 63%) as a white solid. MS (ESI) m/z 278 [M+H]⁺

Step 4. A mixture of methyl 4-iodo-5-methylpicolinate (556 mg, 2 mmol) and sodium hydroxide (160 mg, 4 mmol) in methanol/water (8 mL/2 mL) was stirred at room temperature for 4 h. The mixture was concentrated in vacuo. The residue was diluted with aqueous hydrochloride acid (0.5 N, 100 mL) and extracted with ethyl acetate (3×100 mL). The combined organic phases were concentrated in vacuo to afford 4-iodo-5-methylpicolinic acid (500 mg, 1.9 mmol, 85%) as a white solid. MS (ESI) m/z 264 [M+H]⁺

Step 5. A mixture of 4-iodo-5-methylpicolinic acid (262 mg, 1 mmol) and copper (I) cyanide (97 mg, 1.1 mmol) in N,N-dimethylformamide (4 mL) was stirred at 110° C. for 16 h. The mixture was concentrated in vacuo. The residue was diluted with water (100 mL) and extracted with ethyl acetate (3×100 mL). The combined organic phases were concentrated in vacuo. The residue was purified with prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-cyano-5-methylpicolinic acid (50 mg, 0.31 mmol, 31%) as a white solid. MS (ESI) m/z 163 [M+H]⁺

Step 6. A mixture of 4-cyano-5-methylpicolinic acid (48 mg, 0.3 mmol), 6-methyl-N1-(3-(9-(tetrahydro-2H-pyran-2-yl)-9H-purin-6-yl)pyridin-2-yl)benzene-1,3-diamine (120 mg, 0.3 mmol), 1-[bis(dimethylamino)methylene]-1H-1,2,3-triazolo[4,5-b]pyridinium 3-oxid hexafluorophosphate (171 mg, 0.45 mmol) and N,N-diisopropylethylamine (77 mg, 0.6 mmol) in N,N-dimethylformamide (4 mL) was stirred at room temperature for 4 h. The mixture was concentrated in vacuo. The residue was diluted with water (100 mL) and extracted with ethyl acetate (3×100 mL). The combined organic phases were concentrated in vacuo. The residue was purified by flash chromatography (silica gel, ethyl acetate/petroleum ether=0% to 80%) to afford 4-cyano-5-methyl-N-(4-methyl-3-((3-(9-(tetrahydro-2H-pyran-2-yl)-9H-purin-6-yl)pyridin-2-yl)amino)phenyl)picolinamide (50 mg, 0.09 mmol, 31%) as a yellow solid.

Step 7. 4-cyano-5-methyl-N-(4-methyl-3-((3-(9-(tetrahydro-2H-pyran-2-yl)-9H-purin-6-yl)pyridin-2-yl)amino)phenyl)picolinamide (54 mg, 0.1 mmol) was dissolved in hydrogen chloride solution (3 M in methanol, 4 mL) and stirred at room temperature for 4 h. The mixture was concentrated in vacuo. The residue was washed with methanol and dried at 60° C. to afford N-(3-((3-(9H-purin-6-yl)pyridin-2-yl)amino)-4-methylphenyl)-4-cyano-5-methylpicolinamide (5 mg, 0.01 mmol, 11%) as a yellow solid.

¹H NMR (400 MHZ, DMSO-d₆) δ 9.75 (s, 1H), 9.10 (s, 1H), 8.88 (s, 1H), 8.81 (s, 1H), 8.68 (s, 1H), 8.37 (s, 2H), 7.47 (d, J=6.4 Hz, 1H), 7.28 (d, J=8.4 Hz, 1H), 7.07 (dd, J=7.9, 4.7 Hz, 1H), 2.60 (s, 3H), 2.44 (s, 3H). MS (ESI) m/z 462 [M+H]⁺

### Example 75. Synthesis of N-(3-((3-(9H-purin-6-yl)pyridin-2-yl)amino)-5-fluoro-4-methylphenyl)-2-(1-fluorocyclopropyl)isonicotinamide (Compound I-72)

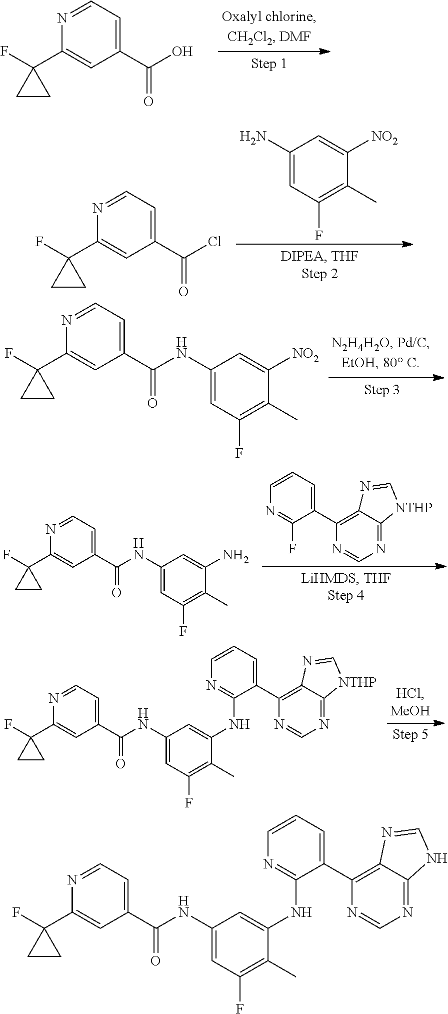

Step 1. To a solution of 2-(1-fluorocyclopropyl)isonicotinic acid (50 mg, 0.28 mmol) in dichloromethane (5 mL) was added oxalyl dichloride (53 mg, 0.41 mmol) and N,N-dimethylformamide (1 drop). The mixture was stirred at room temperature for 2 h. The resulting mixture was concentrated under reduced pressure to give 2-(1-fluorocyclopropyl)isonicotinoyl chloride (60 mg, crude) as light yellow oil, which used without further purification. MS (ESI) m/z 195.1 [M+H]⁺ (methyl ester)

Step 2. To a solution of 3-fluoro-4-methyl-5-nitroaniline (47 mg, 0.28 mmol) in tetrahydrofuran (5 mL) was added N,N-diisopropylethylamine (72 mg, 0.56 mmol) and 2-(1-fluorocyclopropyl)isonicotinoyl chloride (60 mg, crude). The mixture was stirred at room temperature for 2 h and quenched with water (10 mL). The resulting mixture was extracted with ethyl acetate, dried over sodium sulfate, filtered and concentrated to give N-(3-fluoro-4-methyl-5-nitrophenyl)-2-(1-fluorocyclopropyl)isonicotinamide (80 mg, 0.24 mmol, 86% over 2 steps) as a light yellow solid. MS (ESI) m/z 333.9 [M+H]⁺

Step 3. To a solution of N-(3-fluoro-4-methyl-5-nitrophenyl)-2-(1-fluorocyclopropyl)isonicotinamide (80 mg, 0.24 mmol) and hydrazine hydrate (1 mL) in ethanol (10 mL) was added palladium (10% on activated carbon, 10 mg). The mixture was stirred at 80° C. for 1 h. The resulting mixture was filtered and concentrated. The residue was triturated with ethanol and dried to give N-(3-amino-5-fluoro-4-methylphenyl)-2-(1-fluorocyclopropyl)isonicotinamide (60 mg, 0.20 mmol, 83%) as a yellow solid. MS (ESI) m/z 304.2 [M+H]⁺

Step 4. To a solution of N-(3-amino-5-fluoro-4-methylphenyl)-2-(1-fluorocyclopropyl)isonicotinamide (60 mg, 0.20 mmol) and 6-(2-fluoropyridin-3-yl)-9-(tetrahydro-2H-pyran-2-yl)-9H-purine (60 mg, 0.20 mmol) in tetrahydrofuran (10 mL) was added lithium hexamethyldisilazide (0.63 mL, 1.0 mmol, 1.6 M in tetrahydrofuran) at 0° C. slowly. The mixture was stirred at room temperature for 1 h. The reaction was quenched with saturated aqueous ammonium chloride solution (20 mL) at 0° C. and extracted with ethyl acetate (30 mL×3). The combined organic layers were washed with brine, dried over sodium sulfate, filtered and concentrated. The residue was purified by column chromatography (30% ethyl acetate in dichloromethane) to give N-(3-fluoro-4-methyl-5-((3-(9-(tetrahydro-2H-pyran-2-yl)-9H-purin-6-yl)pyridin-2-yl)amino)phenyl)-2-(1-fluorocyclopropyl)isonicotinamide (60 mg, 0.10 mmol, 52%) as a yellow solid. MS (ESI) m/z 583.2 [M+H]⁺

Step 5. A mixture of N-(3-fluoro-4-methyl-5-((3-(9-(tetrahydro-2H-pyran-2-yl)-9H-purin-6-yl)pyridin-2-yl)amino)phenyl)-2-(1-fluorocyclopropyl)isonicotinamide (60 mg, 0.10 mmol) in hydrogen chloride solution (3 M in methanol, 10 mL) was stirred at room temperature for 3 h. The resulting mixture was concentrated, quenched with ammonia/methanol solution and concentrated again. The residue was triturated with methanol and dried to give N-(3-((3-(9H-purin-6-yl)pyridin-2-yl)amino)-5-fluoro-4-methylphenyl)-2-(1-fluorocyclopropyl)isonicotinamide (8.0 mg, 0.017 mmol, 24%) as a light yellow solid. ¹H NMR (400 MHZ, DMSO-d₆) δ 13.87 (s, 1H), 12.47 (s, 1H), 10.70 (s, 1H), 9.81 (s, 1H), 9.10 (s, 1H), 8.79-8.66 (m, 2H), 8.57 (s, 1H), 8.39 (dd, J=4.7, 1.8 Hz, 1H), 8.08 (s, 1H), 7.78 (d, J=5.1 Hz, 1H), 7.54 (d, J=12.1 Hz, 1H), 7.09 (dd, J=7.9, 4.7 Hz, 1H), 2.36 (s, 3H), 1.66-1.53 (m, 2H), 1.45-1.37 (m, 2H). MS (ESI) m/z 498.8 [M+H]⁺

### Example 76. Synthesis of N-(3-((3-(9H-purin-6-yl)pyridin-2-yl)amino)-4-methylphenyl)benzo[d][1,3]dioxole-5-carboxamide (Compound I-74)

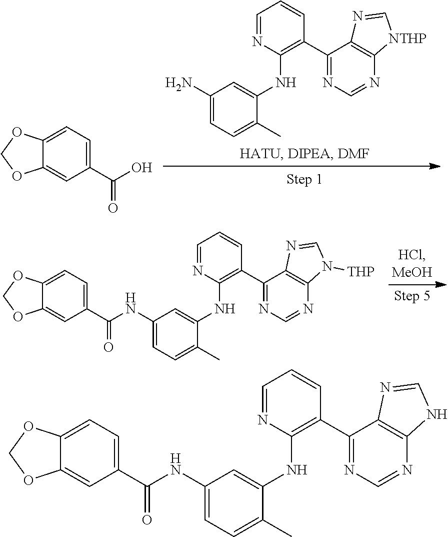

Step 1. A mixture of benzo[d][1,3]dioxole-5-carboxylic acid (37 mg, 0.22 mmol), 6-methyl-N1-(3-(9-(tetrahydro-2H-pyran-2-yl)-9H-purin-6-yl)pyridin-2-yl)benzene-1,3-diamine (82 mg, 0.20 mmol), 1-[bis(dimethylamino)methylene]-1H-1,2,3-triazolo[4,5-b]pyridinium 3-oxid hexafluorophosphate (85 mg, 0.22 mmol) and N,N-diisopropylethylamine (52 mg, 0.4 mmol) in N,N-dimethylformamide (6 mL) was stirred at 25° C. for 2 h. The reaction mixture was extracted with ethyl acetate (10 mL×3). The combined organic layers were washed with brine (20 mL), dried over sodium sulfate, filtered and concentrated. The residue was purified by flash chromatography (petroleum ether/ethyl acetate=1/1) to give N-(4-methyl-3-((3-(9-(tetrahydro-2H-pyran-2-yl)-9H-purin-6-yl)pyridin-2-yl)amino)phenyl)benzo[d][1,3]dioxole-5-carboxamide (100 mg, 0.182 mmol, 91%) as a yellow solid. MS (ESI) m/z 550.2 [M+H]⁺

Step 2. A solution of N-(4-methyl-3-((3-(9-(tetrahydro-2H-pyran-2-yl)-9H-purin-6-yl)pyridin-2-yl)amino)phenyl)benzo[d][1,3]dioxole-5-carboxamide (100 mg, 0.182 mmol) in hydrogen chloride solution (4 M in dioxane, 6 mL) was stirred at 25° C. for 1 h. The solvent was removed under reduced pressure. Ammonia (7 M in methanol, 5 mL) was added and the solvent was removed once again. The residue was washed with ethanol (20 mL) and water (5 mL) to give N-(3-((3-(9H-purin-6-yl)pyridin-2-yl)amino)-4-methylphenyl)benzo[d][1,3]dioxole-5-carboxamide (15.7 mg, 0.033 mmol, 19%) as a yellow solid. ¹H NMR (400 MHZ, DMSO-d₆) δ 12.27 (s, 1H), 10.01 (s, 1H), 9.78 (d, J=7.5 Hz, 1H), 9.07 (s, 1H), 8.70 (s, 1H), 8.62 (s, 1H), 8.34 (d, J=2.8 Hz, 1H), 7.59 (d, J=7.9 Hz, 1H), 7.54 (s, 1H), 7.44 (d, J=6.5 Hz, 1H), 7.21 (d, J=8.4 Hz, 1H), 7.10-6.95 (m, 2H), 6.13 (s, 2H), 2.42 (s, 3H). MS (ESI) m/z 466.1 [M+H]⁺

### Example 77. Synthesis of N-(3-((3-(9H-purin-6-yl)pyridin-2-yl)amino)-4-methylphenyl)-2-(2-(trifluoromethyl)tetrahydro-2H-pyran-4-yl)acetamide (Compound I-75)

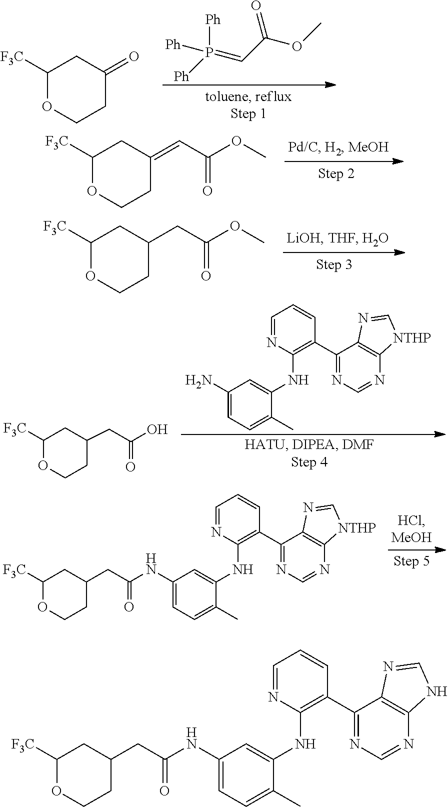

Step 1. A mixture of 2-(trifluoromethyl)dihydro-2H-pyran-4(3H)-one (42 mg, 0.25 mmol) and methyl (triphenylphosphoranylidene)acetate (167 g, 0.5 mmol) in toluene (12 mL) was heated at reflux for 16 h. The reaction mixture was concentrated in vacuo. The crude residue was purified by flash chromatography (petroleum ether/ethyl acetate=100/8) to afford (E)-methyl 2-(2-(trifluoromethyl)-2H-pyran-4(3H,5H,6H)-ylidene)acetate (40 mg, 0.178 mmol, 71%) as a yellow oil.

Step 2. A mixture of (E)-methyl 2-(2-(trifluoromethyl)-2H-pyran-4(3H,5H,6H)-ylidene)acetate (40 mg, 0.18 mmol) and palladium (10% on activated carbon, 20 mg) in methanol (5 mL) was stirred at 20° C. for 5 h. The mixture was filtered and the filtrate was concentrated to give crude methyl 2-(2-(trifluoromethyl)tetrahydro-2H-pyran-4-yl)acetate (40 mg) as a colorless oil. MS (ESI) m/z 227.1 [M+H]⁺

Step 3. To a solution of methyl 2-(2-(trifluoromethyl)tetrahydro-2H-pyran-4-yl)acetate (40 mg, 0.177 mmol) in tetrahydrofuran (1 mL) were added lithium hydroxide hydrate (37 mg, 0.89 mmol) and water (1 mL), and the mixture was stirred at 20° C. for 5 h. The solvent was evaporated under reduced pressure, and the residue was diluted with water (10 mL). The mixture was adjusted to pH 3 by adding 1 N hydrochloric acid slowly. The mixture was extracted with ethyl acetate (30 mL×2). The combined organic layer was dried over sodium sulfate and filtered. The filtrate was concentrated under reduced pressure to give 2-(2-(trifluoromethyl)tetrahydro-2H-pyran-4-yl)acetic acid (18 mg, 0.084 mmol, 47%) as a colorless oil. MS (ESI) m/z 213.1 [M+H]⁺

Step 4. A solution of 6-methyl-N1-(3-(9-(tetrahydro-2H-pyran-2-yl)-9H-purin-6-yl)pyridin-2-yl)benzene-1,3-diamine (325 mg, 0.81 mmol), 2-(2-(trifluoromethyl)tetrahydro-2H-pyran-4-yl)acetic acid (205 mg, 0.97 mmol), 1-[bis(dimethylamino)methylene]-1H-1,2,3-triazolo[4,5-b]pyridinium 3-oxid hexafluorophosphate (555 mg, 1.46 mmol) and N,N-diisopropylethylamine (313 mg, 2.43 mmol) in N,N-dimethylformamide (10 mL) was stirred at 25° C. for 1 h. The mixture was diluted with ethyl acetate (50 mL) and washed with water (50 mL). The organic phase was washed with brine (40 mL×3), dried over sodium sulfate, filtered and concentrated. The residue was purified by flash chromatography (ethyl acetate/petroleum=65/100) to afford N-(4-methyl-3-(3-(9-(tetrahydro-2H-pyran-2-yl)-9H-purin-6-yl)pyridin-2-ylamino)phenyl)-2-(2-(trifluoromethyl)tetrahydro-2H-pyran-4-yl)acetamide (460 mg, 0.77 mmol, 95%) as a yellow solid. MS (ESI) m/z 596.0 [M+H]⁺

Step 5. To a solution of N-(4-methyl-3-(3-(9-(tetrahydro-2H-pyran-2-yl)-9H-purin-6-yl)pyridin-2-ylamino)phenyl)-2-(2-(trifluoromethyl)tetrahydro-2H-pyran-4-yl)acetamide (460 mg, 0.77 mmol) in methanol (15 mL) was added hydrogen chloride solution (3 M in methanol, 3 mL) and the mixture was stirred for 1 h. The reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-2-(2-(trifluoromethyl)tetrahydro-2H-pyran-4-yl)acetamide (230 mg, 0.45 mmol, 58%) as a yellow solid. ¹H NMR (400 MHZ, DMSO-d₆) δ 13.86 (s, 1H), 12.29 (s, 1H), 9.94-9.71 (m, 2H), 9.07 (s, 1H), 8.73 (s, 1H), 8.45 (s, 1H), 8.34 (s, 1H), 7.39 (d, J=6.8 Hz, 1H), 7.16 (d, J=8.2 Hz, 1H), 7.04 (dd, J=7.8, 4.7 Hz, 1H), 4.30-3.47 (m, 3H), 2.41-2.26 (m, 5H), 2.20-2.05 (m, 1H), 1.88-1.57 (m, 2H), 1.36-1.06 (m, 2H). MS (ESI) m/z 512.0 [M+H]⁺

### Example 78. Synthesis of N-(3-((3-(9H-purin-6-yl)pyridin-2-yl)amino)-4-methylphenyl)-2-(2-(trifluoromethyl)piperidin-4-yl)acetamide (Compound I-76)

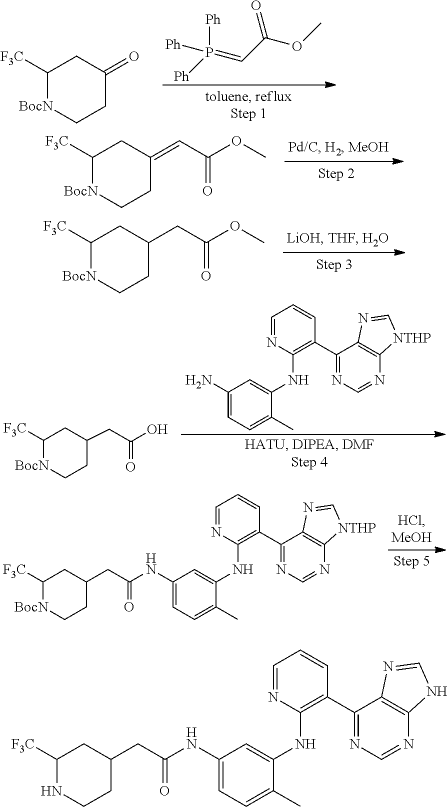

Step 1. A mixture of (tert-butyl 4-oxo-2-(trifluoromethyl)piperidine-1-carboxylate (650 mg, 2.43 mmol) and methyl (triphenylphosphoranylidene)acetate (1.62 g, 4.86 mmol) in toluene (15 mL) was heated at reflux for 16 h. The mixture reaction was concentrated in vacuo. The crude residue was purified by flash chromatography (petroleum ether/ethyl acetate=100/8) to give (E)-tert-butyl 4-(2-methoxy-2-oxoethylidene)-2-(trifluoromethyl)piperidine-1-carboxylate (690 mg, 2.13 mmol, 88%) as a colorless oil. MS (ESI) m/z 224.1 [M−100+H]⁺

Step 2. A mixture of (E)-tert-butyl 4-(2-methoxy-2-oxoethylidene)-2-(trifluoromethyl)piperidine-1-carboxylate (760 mg, 2.35 mmol) and palladium (10% on activated carbon, 304 mg) in methanol (40 mL) was stirred at 25° C. for 16 h. The mixture was filtered and the filtrate was concentrated to give tert-butyl 4-(2-methoxy-2-oxoethyl)-2-(trifluoromethyl)piperidine-1-carboxylate (688 mg, 2.11 mmol, 90%) as a colorless oil. MS (ESI) m/z 270.1 [M+H−56]⁺

Step 3. To a solution of tert-butyl 4-(2-methoxy-2-oxoethyl)-2-(trifluoromethyl)piperidine-1-carboxylate (390 mg, 1.2 mmol) in tetrahydrofuran (5 mL) were added lithium hydroxide hydrate (252 mg, 6.0 mmol) and water (5 mL), and the mixture was stirred at 25° C. for 16 h. The solvent was evaporated under reduced pressure, and the residue was diluted with water (10 mL). The mixture was adjusted to pH 3 by adding 1 N hydrochloric acid slowly. The mixture was extracted with ethyl acetate (60 mL×2). The combined organic layer was dried over sodium sulfate and filtered. The filtrate and concentrated under reduced pressure to give 2-(1-(tert-butoxycarbonyl)-2-(trifluoromethyl)piperidin-4-yl)acetic acid (336 mg, 1.08 mmol, 90%) as a yellow oil. MS (ESI) m/z 256.1 [M+H−56]⁺

Step 4. A solution of 6-methyl-N1-(3-(9-(tetrahydro-2H-pyran-2-yl)-9H-purin-6-yl)pyridin-2-yl)benzene-1,3-diamine (333 mg, 0.83 mmol), 2-(1-(tert-butoxycarbonyl)-2-(trifluoromethyl)piperidin-4-yl)acetic acid (336 mg, 1.08 mmol), 1-[bis(dimethylamino)methylene]-1H-1,2,3-triazolo[4,5-b]pyridinium 3-oxid hexafluorophosphate (631 mg, 1.66 mmol) and N,N-diisopropylethylamine (428 mg, 3.32 mmol) in N,N-dimethylformamide (5 mL) was stirred at 25° C. for 1 h. The mixture was diluted with ethyl acetate (100 mL) and washed with water (100 mL). The organic phase was washed with brine (50 mL×3), dried over sodium sulfate, filtered and concentrated. The residue was purified by flash chromatography (ethyl acetate/petroleum ether=50/100) to afford tert-butyl 4-(2-(4-methyl-3-(3-(9-(tetrahydro-2H-pyran-2-yl)-9H-purin-6-yl)pyridin-2-ylamino)phenylamino)-2-oxoethyl)-2-(trifluoromethyl)piperidine-1-carboxylate (400 mg, 0.58 mmol, 70%) as a yellow solid. MS (ESI) m/z 694.7 [M+H]⁺

Step 5. To a solution of tert-butyl 4-(2-(4-methyl-3-(3-(9-(tetrahydro-2H-pyran-2-yl)-9H-purin-6-yl)pyridin-2-ylamino)phenylamino)-2-oxoethyl)-2-(trifluoromethyl)piperidine-1-carboxylate (95 mg, 0.136 mmol) in methanol (6 mL) was added hydrogen chloride solution (3 M in methanol, 3 mL) and the mixture was stirred for 10 h. The reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-2-(2-(trifluoromethyl)piperidin-4-yl)acetamide (30 mg, 0.058 mmol, 43%) as a yellow solid.

¹H NMR (400 MHZ, DMSO-d₆) δ 13.85 (s, 1H), 12.27 (s, 1H), 9.91-9.79 (m, 2H), 9.07 (s, 1H), 8.71 (s, 1H), 8.43 (s, 1H), 8.37-8.25 (m, 1H), 7.38 (d, J=7.7 Hz, 1H), 7.15 (d, J=6.6 Hz, 1H), 7.03 (dd, J=7.9, 4.7 Hz, 1H), 3.55-3.14 (m, 1H), 3.03-2.94 (m, 1H), 2.79-2.73 (m, 1H), 2.43-2.17 (m, 6H), 2.01-1.43 (m, 3H), 1.29-0.94 (m, 2H). MS (ESI) m/z 510.8 [M+H]⁺

### Example 79. Synthesis of N-(3-((3-(9H-purin-6-yl)pyridin-2-yl)amino)-4-methylphenyl)-2-(3-(trifluoromethyl)piperidin-1-yl)acetamide (Compound I-77)

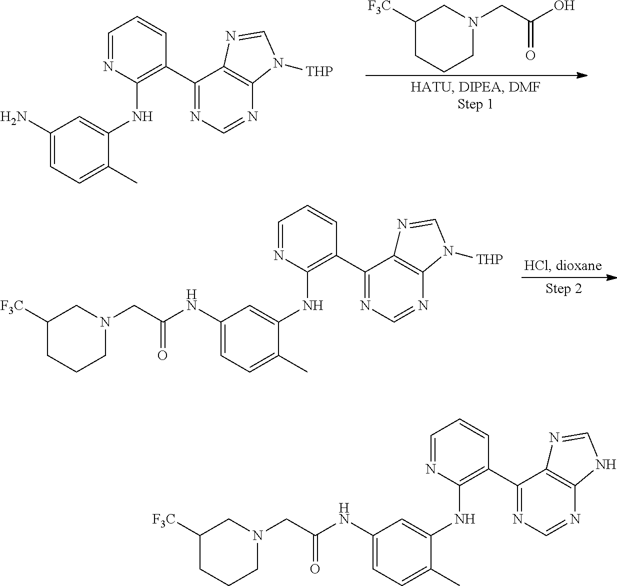

Step 1. To a mixture of 2-(3-(trifluoromethyl)piperidin-1-yl)acetic acid (130 mg, crude) in N,N-dimethylformamide (3 mL) was added 1-[bis(dimethylamino)methylene]-1H-1,2,3-triazolo[4,5-b]pyridinium 3-oxid hexafluorophosphate (281 mg, 0.74 mmol) and N,N-diisopropylethylamine (119 mg, 0.92 mmol). After stirring at room temperature for 5 min, 6-(5-amino-2-methylphenyl)-N-methylpyrido[2,3-d]pyrimidin-2-amine (247 mg, 0.62 mmol) was added. The mixture was stirred at room temperature for another 3 h. The resulting mixture was diluted with water (20 mL) and extracted with ethyl acetate (20 mL×3). The combined organic phases were washed with brine, dried over sodium sulfate, filtered and concentrated. The residue was purified by flash chromatography (petroleum ether/ethyl acetate=1/1) to give N-(4-methyl-3-((3-(9-(tetrahydro-2H-pyran-2-yl)-9H-purin-6-yl)pyridin-2-yl)amino)phenyl)-2-(3-(trifluoromethyl)piperidin-1-yl)acetamide (180 mg, 0.30 mmol, 49%) as a yellow solid. MS (ESI) m/z 595.2 [M+H]⁺

Step 2. A solution of N-(4-methyl-3-((3-(9-(tetrahydro-2H-pyran-2-yl)-9H-purin-6-yl)pyridin-2-yl)amino)phenyl)-2-(3-(trifluoromethyl)piperidin-1-yl)acetamide (100 mg, 0.17 mmol) in hydrogen chloride solution (4 M in dioxane, 3 mL) was stirred at room temperature for 1 h. The solvent was removed under reduced pressure and the residue was treated with ammonia (2 mL, 8 M in methanol). The resulting mixture was filtered and the solid was collected. The solid was dried under reduced pressure to give N-(3-((3-(9H-purin-6-yl)pyridin-2-yl)amino)-4-methylphenyl)-2-(3-(trifluoromethyl)piperidin-1-yl)acetamide (45.7 mg, 0.09 mmol, 53%) as a yellow solid. ¹H NMR (400 MHZ, DMSO-d₆) δ 12.25 (br s, 1H), 11.96 (s, 1H), 9.78 (d, J=7.6 Hz, 1H), 9.03 (s, 1H), 8.64-8.63 (m, 2H), 8.36-8.34 (m, 1H), 7.89 (s, 1H), 7.15 (d, J=8.4 Hz, 1H), 7.02-7.00 (m, 1H), 6.93-6.90 (m, 1H), 3.30-3.19 (m, 2H), 3.10 (d, J=8.0 Hz, 1H), 2.97-2.94 (m, 1H), 2.45-2.39 (m, 2H), 2.34-2.29 (m, 4H), 2.01-1.98 (m, 1H), 1.91-1.87 (m, 1H), 1.72-1.64 (m, 1H), 1.48-1.42 (m, 1H). MS (ESI) m/z 511.1 [M+H]⁺

### Example 80. Synthesis of N-(3-((3-(9H-purin-6-yl)pyridin-2-yl)amino)-4-methylphenyl)-2-(3-(trifluoromethyl)-1H-1,2,4-triazol-1-yl)acetamide (Compound I-78)

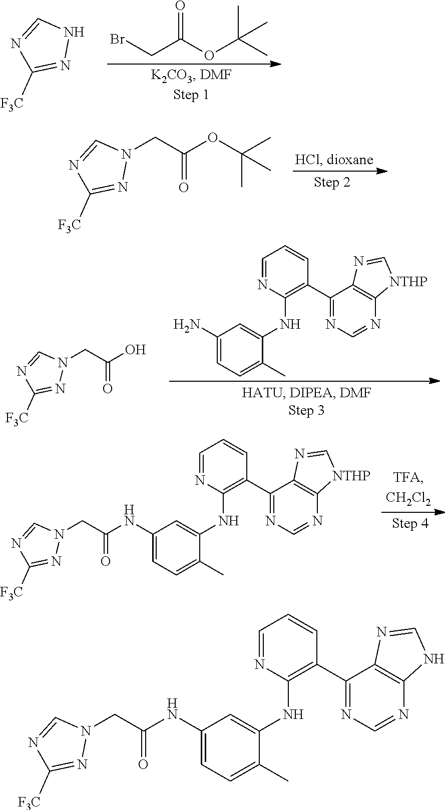

Step 1. To a mixture of 3-(trifluoromethyl)-1H-1,2,4-triazole (240 mg, 1.75 mmol) and tert-butyl 2-bromoacetate (444 mg, 2.28 mmol) in N,N-dimethylformamide (10 mL) was added potassium carbonate (724 mg, 5.25 mmol). The mixture was stirred at room temperature for 2 h. The reaction mixture was diluted with water (20 mL) and extracted with dichloromethane (30 mL×3). The organic layer was dried over anhydrous sodium sulfate, filtered and concentrated in vacuo. The residue was purified by flash chromatography (petroleum ether/ethyl acetate=10/1 to 1/1) to afford tert-butyl 2-(3-(trifluoromethyl)-1H-1,2,4-triazol-1-yl)acetate as a light yellow oil (300 mg, 1.19 mmol, 68%). ¹HNMR (400 MHZ, CDCl₃) δ8.29 (s, 1H), 4.93 (s, 2H), 1.49 (s, 9H).

Step 2. To a solution of tert-butyl 2-(3-(trifluoromethyl)-1H-1,2,4-triazol-1-yl)acetate (250 mg, 1.0 mmol) in 1,4-dioxane (10 mL) was added hydrogen chloride solution (4 M in 1,4-dioxane, 10 mL). The mixture was stirred at room temperature for 16 h. The reaction mixture was concentrated in vacuo to afford crude 2-(3-(trifluoromethyl)-1H-1,2,4-triazol-1-yl)acetic acid (200 mg) as a light yellow oil, which was without further purification. ¹HNMR (400 MHZ, CDCl₃) δ8.41 (s, 1H), 5.08 (s, 2H).

Step 3. To a mixture of 6-methyl-N1-(3-(9-(tetrahydro-2H-pyran-2-yl)-9H-purin-6-yl)pyridin-2-yl)benzene-1,3-diamine (100 mg, 0.25 mmol), 2-(3-(trifluoromethyl)-1H-1,2,4-triazol-1-yl)acetic acid (48 mg, 0.25 mmol) and 1-[bis(dimethylamino)methylene]-1H-1,2,3-triazolo[4,5-b]pyridinium 3-oxid hexafluorophosphate (142 mg, 0.38 mmol) in N,N-dimethylformamide (5 mL) was added N,N-diisopropylethylamine (161 mg, 1.25 mmol). The mixture was stirred at room temperature for 2 h. The reaction mixture was diluted with water (20 mL) and extracted with ethyl acetate (50 mL×3). The organic layer was dried over sodium sulfate, filtered and concentrated in vacuo. The residue was purified by flash chromatography (petroleum ether/ethyl acetate=10/1 to 1/1) to afford N-(4-methyl-3-((3-(9-(tetrahydro-2H-pyran-2-yl)-9H-purin-6-yl)pyridin-2-yl)amino)phenyl)-2-(3-(trifluoromethyl)-1H-1,2,4-triazol-1-yl)acetamide (80 mg, 0.14 mmol, 55%) as a light yellow solid. MS (ESI) m/z 577.2 [M−H]⁻

Step 4. Trifluoroacetic acid (5 mL) was added to a solution of N-(4-methyl-3-((3-(9-(tetrahydro-2H-pyran-2-yl)-9H-purin-6-yl)pyridin-2-yl)amino)phenyl)-2-(3-(trifluoromethyl)-1H-1,2,4-triazol-1-yl)acetamide (60 mg, 0.10 mmol) in dichloromethane (5 mL) at 0° C. The reaction mixture was stirred at room temperature for 16 h. The reaction mixture was concentrated in vacuo and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-2-(3-(trifluoromethyl)-1H-1,2,4-triazol-1-yl)acetamide (32.4 mg, 0.065 mmol, 65%) as a light yellow solid. ¹HNMR (400 MHZ, DMSO-d₆) δ 12.40 (s, 1H), 10.45 (s, 1H), 9.81-9.79 (d, J=7.6 Hz, 1H), 9.05 (s, 1H), 8.89 (s, 1H), 8.69 (s, 1H), 8.58-8.58 (d, J=2.0 Hz, 1H), 8.37-8.35 (m, 1H), 7.36-7.33 (m, 1H), 7.21-7.19 (d, J=8.0 Hz, 1H), 7.06-7.03 (m, 1H), 5.30 (s, 3H), 2.43 (s, 3H). MS (ESI) m/z 495.1 [M+H]⁺

### Example 81. Synthesis of 3-((3-(9H-purin-6-yl)pyridin-2-yl)amino)-N-(3-(tert-butyl)-1-cyclopropyl-1H-pyrazol-5-yl)-4-methylbenzamide (Compound I-79)

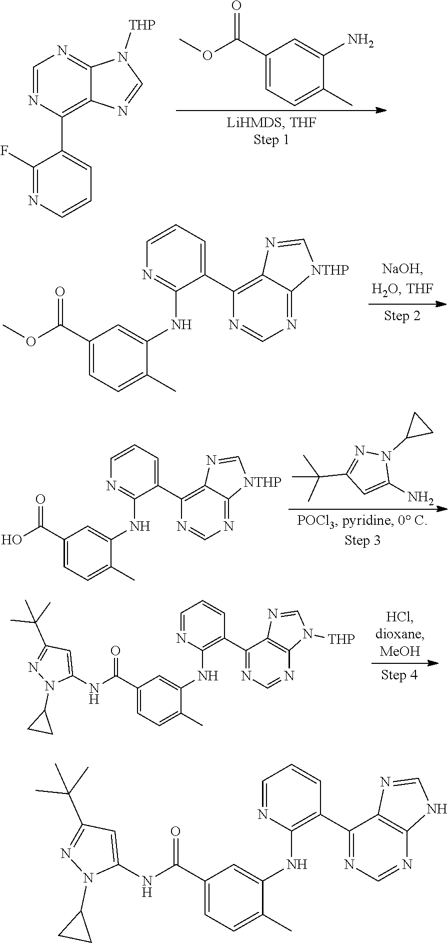

Step 1. To a solution of 6-(2-fluoropyridin-3-yl)-9-(tetrahydro-2H-pyran-2-yl)-9H-purine (500 mg, 1.67 mmol) and methyl 3-amino-4-methylbenzoate in (276 mg, 1.67 mmol) in tetrahydrofuran (45 mL) was added lithium hexamethyldisilazide (1.6 M in tetrahydrofuran, 2.6 mL, 4.18 mmol) at 0° C. slowly. The mixture was stirred at 20° C. for 1 h. The reaction was quenched with saturated aqueous ammonium chloride solution (25 mL) at 0° C. and extracted with ethyl acetate (30 mL×3). The combined organic layer was washed with brine (30 mL), dried over sodium sulfate and filtered. The filtrate was concentrated under reduced pressure. The residue was purified by flash chromatography (petroleum ether/ethyl acetate=2/3) to give methyl 4-methyl-3-((3-(9-(tetrahydro-2H-pyran-2-yl)-9H-purin-6-yl)pyridin-2-yl)amino)benzoate (137 mg, 0.30 mmol, 19%) as a yellow solid. MS (ESI) m/z 445.0 [M+H]⁺

Step 2. To a mixture of methyl 4-methyl-3-((3-(9-(tetrahydro-2H-pyran-2-yl)-9H-purin-6-yl)pyridine-2-yl)amino)benzoate (130 mg, 0.29 mmol) in tetrahydrofuran (6 mL) was added aqueous sodium hydroxide solution (2 mL, 2.9 mmol, 2 N), and the mixture was stirred at room temperature for 3 days. The reaction mixture was adjusted to pH=5.0 with hydrochloric acid (6 N). The solution was filtered to give 4-methyl-3-((3-(9-(tetrahydro-2H-pyran-2-yl)-9H-purin-6-yl)pyridin-2-yl)amino)benzoic acid (60 mg, 0.14 mmol, 48%) as a yellow solid. MS (ESI) m/z 431.1 [M+H]⁺

Step 3. To a mixture of 4-methyl-3-((3-(9-(tetrahydro-2H-pyran-2-yl)-9H-purin-6-yl)pyridin-2-yl)amino)benzoic acid (60 mg, 0.14 mmol) and 3-(tert-butyl)-1-cyclopropyl-1H-pyrazol-5-amine (25 mg, 0.14 mmol) dissolved in pyridine (2 mL) was added phosphorus(V) oxychloride (21 mg, 25.0 mmol), and the mixture was stirred at room temperature for 2 h. The mixture was diluted with ethyl acetate (30 mL) and washed with saturated sodium bicarbonate (10 mL) and brine (25 mL). The organic layer was dried over sodium sulfate, filtered and concentrated. The residue was purified by flash chromatography (ethyl acetate) to give N-(3-(tert-butyl)-1-cyclopropyl-1H-pyrazol-5-yl)-4-methyl-3-((3-(9-(tetrahydro-2H-pyran-2-yl)-9H-purin-6-yl)pyridin-2-yl)amino)benzamide (20 mg, 0.034 mmol, 24%) as a yellow solid. MS (ESI) m/z 591.9 [M+H]⁺

Step 4. A solution of N-(3-(tert-butyl)-1-cyclopropyl-1H-pyrazol-5-yl)-4-methyl-3-((3-(9-(tetrahydro-2H-pyran-2-yl)-9H-purin-6-yl)pyridin-2-yl)amino)benzamide (20 mg, 0.04 mmol) in hydrogen chloride solution (4 M in dioxane, 2 mL) and methanol (1 mL) was stirred at room temperature for 2 h. The mixture was concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give 3-((3-(9H-purin-6-yl)pyridin-2-yl)amino)-N-(3-(tert-butyl)-1-cyclopropyl-1H-pyrazol-5-yl)-4-methylbenzamide (2.2 mg, 0.004 mmol, 14%) as a yellow solid. ¹H NMR (400 MHZ, DMSO-d₆) δ 12.66 (s, 1H), 10.05 (s, 1H), 9.86 (d, J=7.6 Hz, 1H), 9.02 (d, J=1.2 Hz, 1H), 9.00 (s, 1H), 8.57-8.56 (m, 1H), 8.34-8.33 (m, 1H), 7.53 (d, J=7.6 Hz, 1.2 Hz, 1H), 7.41 (d, J=8.0 Hz, 1H), 7.07-7.04 (m, 1H), 6.15 (s, 1H), 3.52-3.44 (m, 1H), 2.55 (s, 3H), 1.23 (s, 9H), 1.01-0.89 (m, 4H). MS (ESI) m/z 507.9 [M+H]⁺

### Example 82. Synthesis of N-(3-((3-(9H-purin-6-yl)pyridin-2-yl)amino)-4-methylphenyl)-3-(dimethylamino)benzamide (Compound I-80)

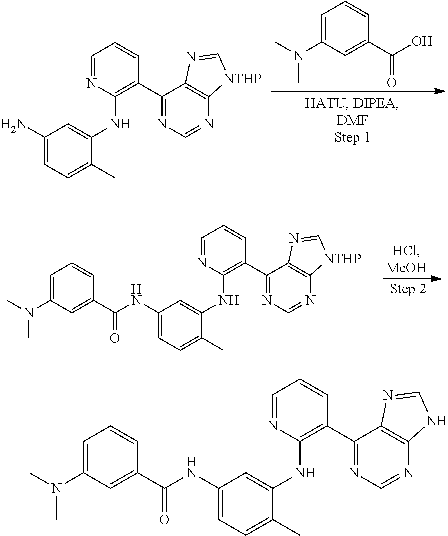

Step 1. A solution of 6-methyl-N1-(3-(9-(tetrahydro-2H-pyran-2-yl)-9H-purin-6-yl)pyridin-2-yl)benzene-1,3-diamine (150 mg, 0.37 mmol), 3-(dimethylamino)benzoic acid (64 mg, 0.39 mmol), 1-[bis(dimethylamino)methylene]-1H-1,2,3-triazolo[4,5-b]pyridinium 3-oxid hexafluorophosphate (213 mg, 0.56 mmol) and N,N-diisopropylethylamine (143 mg, 1.11 mmol) in N,N-dimethylformamide (5 mL) was stirred at 25° C. for 16 h. The mixture was diluted with ethyl acetate (100 mL) and washed with water (100 mL). The organic phase was washed with brine (50 mL×3), dried over sodium sulfate, filtered and concentrated. The residue was purified by flash chromatography (ethyl acetate/petroleum ether=40/100) to give tert-butyl 3-(dimethylamino)-N-(4-methyl-3-(3-(9-(tetrahydro-2H-pyran-2-yl)-9H-purin-6-yl)pyridin-2-ylamino)phenyl)benzamide (170 mg, 0.31 mmol, 84%) as a yellow solid. MS (ESI) m/z 549.2 [M+H]⁺

Step 2. To a solution of 3-(dimethylamino)-N-(4-methyl-3-(3-(9-(tetrahydro-2H-pyran-2-yl)-9H-purin-6-yl)pyridin-2-ylamino)phenyl)benzamide (170 mg, 0.31 mmol) in methanol (20 mL) was added hydrogen chloride solution (3 M in methanol, 5 mL) and the mixture was stirred for 2 h. After completed, the reaction mixture was concentrated and the residue was diluted with methanol (50 mL) The pH was adjusted to 8 with ammonia (7 N in methanol). The mixture was concentrated under reduced pressure. The residue was treated with methanol (10 mL) and filtered. The solid was suspended in water (50 mL) and lyophilized under vacuum to afford N-(3-((3-(9H-purin-6-yl)pyridin-2-yl)amino)-4-methylphenyl)-3-(dimethylamino)benzamide (92.9 mg, 0.2 mmol, 65%) as a yellow solid. ¹H NMR (400 MHZ, DMSO-d₆) δ 13.86 (s, 1H), 12.28 (s, 1H), 10.07 (s, 1H), 9.83 (s, 1H), 9.09 (s, 1H), 8.72 (s, 1H), 8.62 (s, 1H), 8.34 (d, J=2.8 Hz, 1H), 7.44 (d, J=8.5 Hz, 1H), 7.27 (dt, J=17.7, 9.7 Hz, 4H), 7.03 (dd, J=7.7, 4.8 Hz, 1H), 6.92 (d, J=7.1 Hz, 1H), 2.97 (s, 6H), 2.42 (s, 3H). MS (ESI) m/z 464.9 [M+H]⁺

### Example 83. Synthesis of N-(3-((3-(9H-purin-6-yl)pyridin-2-yl)amino)-4-methylphenyl)-4-chloro-3-(dimethylamino)benzamide (Compound I-81)

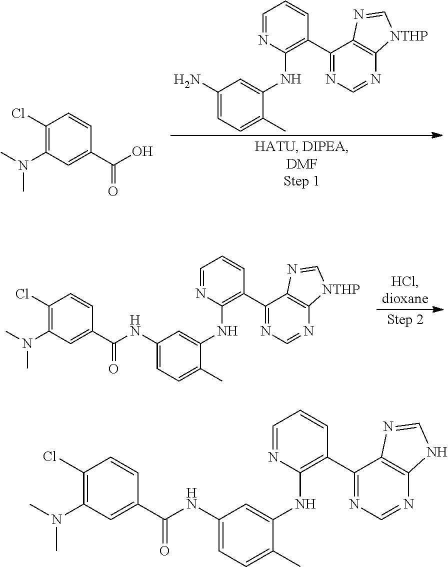

Step 1. To a mixture of 4-chloro-3-(dimethylamino)benzoic acid (50 mg, 0.25 mmol) in N,N-dimethylformamide (5 mL) was added 6-methyl-N1-(3-(9-(tetrahydro-2H-pyran-2-yl)-9H-purin-6-yl)pyridin-2-yl)benzene-1,3-diamine (50 mg, 0.12 mmol), 1-[bis(dimethylamino)methylene]-1H-1,2,3-triazolo[4,5-b]pyridinium 3-oxid hexafluorophosphate (80 mg, 0.21 mmol) and N,N-diisopropylethylamine (36 mg, 0.28 mmol). The reaction mixture stirred at room temperature for 2 h. The mixture was diluted with water (10 mL) and extracted with ethyl acetate (10 mL×3). The combined organic layer was dried over sodium sulfate, filtered and concentrated to give 4-chloro-3-(dimethylamino)-N-(4-methyl-3-((3-(9-(tetrahydro-2H-pyran-2-yl)-9H-purin-6-yl)pyridin-2-yl)amino)phenyl)benzamide (120 mg, crude) as yellow solid. MS (ESI) m/z 583.2 [M+H]⁺

Step 2. A solution of 4-chloro-3-(dimethylamino)-N-(4-methyl-3-((3-(9-(tetrahydro-2H-pyran-2-yl)-9H-purin-6-yl)pyridin-2-yl)amino)phenyl)benzamide (120 mg, crude) and hydrogen chloride solution (2 M in 1,4-dioxane, 5 mL) and stirred room temperature for 3 h. The mixture was concentrated in vacuo. The residue was diluted with saturated sodium bicarbonate aqueous solution (30 mL) and extracted with ethyl acetate (20 mL×3). The combined organic phases were concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-4-chloro-3-(dimethylamino)benzamide (42.1 mg, 0.036 mmol, 41%) as a yellow solid. ¹H NMR (400 MHZ, DMSO-d₆) δ 13.86 (s, 1H), 12.30 (s, 1H), 10.22 (s, 1H), 9.81 (s, 1H), 9.09 (s, 1H), 8.86-8.22 (m, 3H), 7.75-6.90 (m, 6H), 2.80 (s, 6H), 2.44 (s, 3H). MS (ESI) m/z 499.0 [M+H]⁺

### Example 84. Synthesis of N-(3-((3-(9H-purin-6-yl)pyridin-2-yl)amino)-4-methylphenyl)-2-(oxepan-2-yl)acetamide (Compound I-82)

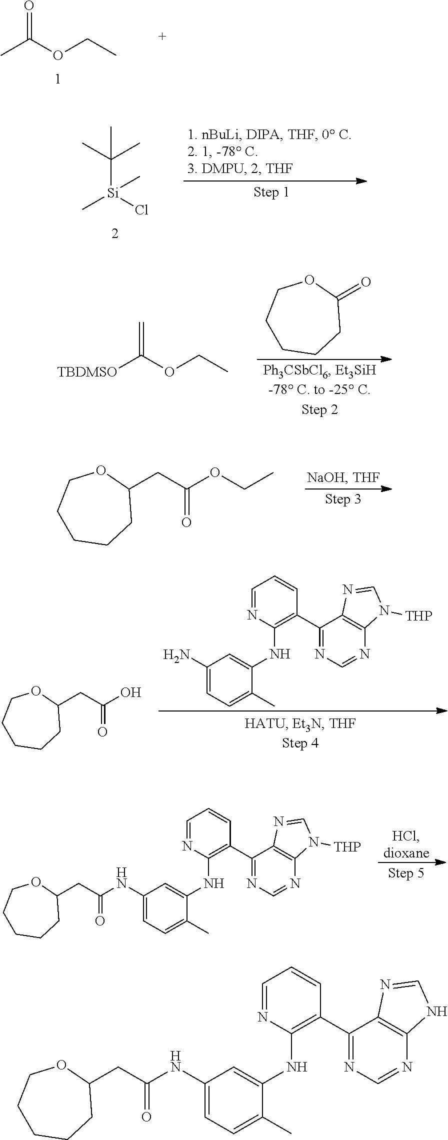

Step 1. To a solution of diisopropylamine (6.06 g, 60 mmol) in tetrahydrofuran (100 mL) was added n-butyllithium (22 mL, 55 mmol, 2.5 M in tetrahydrofuran) at 0° C. The mixture was stirred at 0° C. for 20 min. Ethyl acetate (5 mL) was added at −78° C., and then stirred at −78° C. for 30 min. 1,3-Dimethyl-3,4,5,6-tetrahydro-2(1H)-pyrimidinone (15 mL, 124 mmol) and tert-butyldimethylsilyl chloride (9 g, 60 mmol) were added at −78° C. The mixture was stirred at ambient temperature for 18 h. The reaction was concentrated. The residue was dissolved in hexane (300 mL), and sequentially washed with water (150 mL), saturated copper(II) sulfate solution (150 mL), saturated sodium bicarbonate solution (150 mL), and brine (150 mL). The organic layer was concentrated. The residue was purified by distillation to get tert-butyl((1-ethoxyvinyl)oxy)dimethylsilane (3.2 g, 15.8 mmol, 32%) as a colorless oil. ¹H NMR (400 MHZ, CDCl₃) δ 3.57 (q, J=7.0 Hz, 2H), 3.04 (d, J=2.4 Hz, 1H), 2.88 (d, J=2.4 Hz, 1H), 1.12 (t, J=7.0 Hz, 3H), 0.76 (d, J=2.8 Hz, 9H), 0.02-0.00 (m, 6H).

Step 2. To a solution of tert-butyl((1-ethoxyvinyl)oxy)dimethylsilane (404 mg, 2 mmol), oxepan-2-one (228 mg, 2 mmol), triethylsilane (348 mg, 3 mmol) in dichloromethane (10 mL) was added Ph₃CSbCl₆ (58 mg, 0.1 mmol) at −78° C. The mixture was stirred at −78° C. for 0.5 h and then at −25° C. for 3 h. The reaction was treated with saturated sodium bicarbonate solution (20 mL), and extracted with ethyl acetate (20 mL×2). The combined organic layers were concentrated. The residue was purified by flash chromatography (petroleum ether/ethyl acetate=10/1) to give ethyl 2-(oxepan-2-yl)acetate (160 mg, 0.86 mmol, 43%) as a colorless oil. MS (ESI) m/z 187.1 [M+H]⁺

Step 3. To a solution of ethyl 2-(oxepan-2-yl)acetate (160 mg, 0.86 mmol) in tetrahydrofuran (1 mL) was added aqueous sodium hydroxide solution (1 mL, 2 mmol, 2 M). The mixture was stirred at room temperature for 2 h. The reaction mixture was adjusted to pH=3.0 with hydrochloric acid (1 N) and extracted with ethyl acetate (10 mL×2). The combined organic layers were concentrated to give 2-(oxepan-2-yl)acetic acid (100 mg, 0.63 mmol, 74%) as a colorless oil. MS (ESI) m/z 159.0 [M+H]⁺

Step 4. A mixture of 6-methyl-N1-(3-(9-(tetrahydro-2H-pyran-2-yl)-9H-purin-6-yl)pyridin-2-yl)benzene-1,3-diamine (100 mg, 0.25 mmol), 2-(oxepan-2-yl)acetic acid (39 mg, 0.25 mmol), 1-[bis(dimethylamino)methylene]-1H-1,2,3-triazolo[4,5-b]pyridinium 3-oxid hexafluorophosphate (114 mg, 0.3 mmol), and triethylamine (76 mg, 0.75 mmol) in tetrahydrofuran (3 mL) was stirred at room temperature for 18 h. The reaction mixture was concentrated. The residue was purified by flash chromatography (dichloromethane/methanol=10/1) to give N-(4-methyl-3-((3-(9-(tetrahydro-2H-pyran-2-yl)-9H-purin-6-yl)pyridin-2-yl)amino)phenyl)-2-(oxepan-2-yl)acetamide (120 mg, 0.22 mmol, 89%) as a yellow solid. MS (ESI) m/z 541.8 [M+H]⁺

Step 5. To a solution of N-(4-methyl-3-((3-(9-(tetrahydro-2H-pyran-2-yl)-9H-purin-6-yl)pyridin-2-yl)amino)phenyl)-2-(oxepan-2-yl)acetamide (100 mg, 0.18 mmol) in methanol (1 mL) was added hydrogen chloride solution (4 M in dioxane, 1 mL). The mixture was stirred at room temperature for 1 h. After completed, the reaction mixture was concentrated and methanol (2 mL) was added. The pH was adjusted to 8 with ammonia (7 N in methanol). The mixture was concentrated. The residue was washed with acetonitrile (5 mL) and then water (5 mL). The solid was suspended in water (20 mL) and lyophilized under vacuum to afford N-(3-((3-(9H-purin-6-yl)pyridin-2-yl)amino)-4-methylphenyl)-2-(oxepan-2-yl)acetamide (29.2 mg, 0.064 mmol, 35%) as a yellow solid. ¹H NMR (400 MHZ, DMSO-d₆) δ 12.34 (s, 1H), 9.82 (s, 2H), 9.08 (s, 1H), 8.74 (s, 1H), 8.33 (dd, J=12.9, 11.2 Hz, 2H), 7.41 (dd, J=8.2, 2.0 Hz, 1H), 7.18 (d, J=8.1 Hz, 1H), 7.07 (dd, J=7.8, 4.8 Hz, 1H), 4.01-3.91 (m, 1H), 3.71 (dd, J=12.1, 4.9 Hz, 1H), 3.50-3.43 (m, 2H), 2.43-2.30 (m, 4H), 1.79 (d, J=11.8 Hz, 1H), 1.75-1.39 (m, 7H). MS (ESI) m/z 458.0 [M+H]⁺

### Example 85. Synthesis of N-(3-((3-(9H-purin-6-yl)pyridin-2-yl)amino)-4-methylphenyl)-2-(1,4-oxazepan-7-yl)acetamide (Compound I-83)

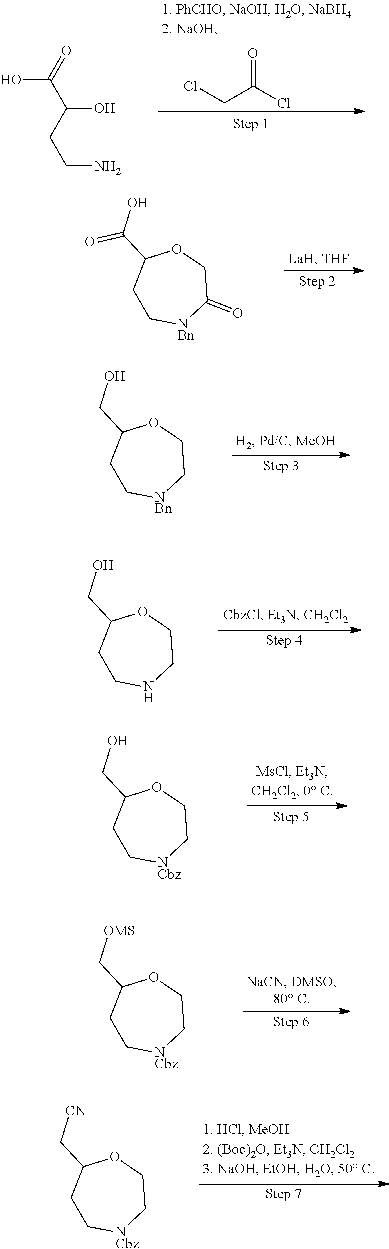

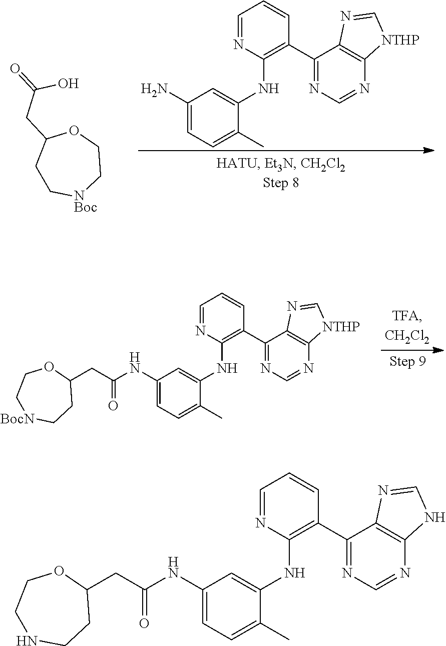

Step 1. To a solution of 4-amino-2-hydroxybutanoic acid (50 g, 419.75 mmol) and sodium hydroxide (18.47 g, 461.72 mmol) in water (450 mL) was added benzaldehyde (102.37 g, 2.30 mmol) at room temperature. The mixture was stirred at room temperature for 30 minutes. The reaction mixture was cooled to 0° C. and sodium borohydride (16.54 g, 461.72 mmol) was added over 30 minutes. Then the reaction mixture was stirred at room temperature for 12 hours. Sodium hydroxide (18.47 g, 464.72 mmol) was added into the resulting mixture, stirred for 30 minutes and then 2-chloroacetyl chloride (49.78 g, 440.73 mmol) was added at 0° C. The mixture was stirred at room temperature for 4 hours. Another batch of sodium hydroxide (184.7 g, 4647.2 mmol) was added at 0° C. and then the mixture was stirred at room temperature for 4 hours. The resulting mixture was acidified with 1N HCl to pH 4, extracted with dichloromethane (1000 mL×3), dried over anhydrous sodium sulfate, filtered and concentrated. The residue was purified by flash chromatography (dichloromethane/methanol=10/1) to give 4-benzyl-3-oxo-1,4-oxazepane-7-carboxylic acid (7.0 g, 28.12 mmol, 6.7%) as a yellow oil. MS (ESI) m/z 250.1 [M+H]⁺

Step 2. To a solution of 4-benzyl-3-oxo-1,4-oxazepane-7-carboxylic acid (7 g, 28.08 mmol) in tetrahydrofuran (100 mL) was slowly added lithium aluminum hydride (3.20 g, 84.25 mmol) at 0° C. The mixture was stirred at room temperature for 6 hours. The reaction was quenched with water (1 mL), filtered and the filtrate was concentrated to give (4-benzyl-1,4-oxazepan-7-yl)methanol (2.0 g, 8.99 mmol, 32%) as a yellow oil. MS (ESI) m/z 222.1 [M+H]⁺

Step 3. A solution of (4-benzyl-1,4-oxazepan-7-yl)methanol (2.0 g. 9.05 mmol) and palladium (10% on active carbon, 0.20 g) in methanol (50 mL) was stirred at room temperature under hydrogen atmosphere for 48 hours. The mixture was filtered and the filtrate was concentrated to give (1,4-oxazepan-7-yl)methanol (1.18 g, 9.05 mmol) as a yellow oil. MS (ESI) m/z 132.1 [M+H]⁺

Step 4. To a solution of (1,4-oxazepan-7-yl)methanol (0.41 g, 3.13 mmol) and triethylamine (0.95 g, 9.38 mmol) in dichloromethane (10 mL) was added benzyl chloroformate (0.48 g, 2.81 mmol) at 0° C. After the addition, the mixture was stirred at room temperature for 2 hours. The reaction was quenched with water (10 mL), extracted with dichloromethane (10 mL×2), dried over anhydrous sodium sulfate, filtered and concentrated to give benzyl 7-(hydroxymethyl)-1,4-oxazepane-4-carboxylate (0.70 g, 2.66 mmol, 85%) as a yellow oil. MS (ESI) m/z 222.3 [M+H]⁺

Step 5. To a solution of 7-(hydroxymethyl)-1,4-oxazepane-4-carboxylate (0.70 g, 2.64 mmol) and triethylamine (0.80 g, 7.92 mmol) in dichloromethane (10 mL) was added methanesulfonyl chloride (0.33 g, 2.90 mmol) at 0° C. After the addition, the mixture was stirred at room temperature for 3 hours. The reaction was quenched with water (10 mL), extracted with dichloromethane (10 mL×2), dried over anhydrous sodium sulfate, filtered and concentrated to give benzyl 7-((methylsulfonyloxy)methyl)-1,4-oxazepane-4-carboxylate (0.60 g, 1.76 mmol, 67%) as a yellow oil. MS (ESI) m/z 344.2 [M+H]⁺

Step 6. A solution of benzyl 7-((methylsulfonyloxy)methyl)-1,4-oxazepane-4-carboxylate (3.00 g, 8.74 mmol) and sodium cyanide (1.70 g, 26.22 mmol) in dimethyl sulfoxide (30 mL) was stirred at 80° C. for 16 hours. The mixture was cooled to room temperature, diluted with water (50 mL), extracted with ethyl acetate (100 mL×2), washed with brine (40 mL×2), dried over anhydrous sodium sulfate, filtered and concentrated to give benzyl 7-(cyanomethyl)-1,4-oxazepane-4-carboxylate (1.50 g, 5.46 mmol, 62%) as a yellow oil. MS (ESI) m/z 275.3 [M+H]⁺

Step 7. A solution of benzyl 7-(cyanomethyl)-1,4-oxazepane-4-carboxylate (1.50 g, 5.46 mmol) in hydrochloric acid (3 M in methanol, 25 mL) was stirred at room temperature for 48 hours. The solvent was removed and the residue was dissolved in dichloromethane (25 mL). Triethylamine (1.65 g, 16.38 mmol) and di-tert-butyl dicarbonate (3.42 g, 16.38 mmol) were added into the resulting mixture. Then the reaction mixture was stirred at room temperature for 2 hours. The solvent was removed and the residue was dissolved in ethanol (50 mL) and water (10 mL). Sodium hydroxide (0.65 g, 16.38 mmol) was added and the reaction mixture was stirred at 50° C. for 6 hours. The mixture was cooled to room temperature and acidified with 1 N HCl to pH 4, then extracted with dichloromethane (100 mL×3), dried over anhydrous sodium sulfate, filtered and concentrated. The residue was purified by flash chromatography (dichloromethane/methanol=10/1) to give 2-(4-(tert-butoxycarbonyl)-1,4-oxazepan-7-yl)acetic acid (0.40 g, 1.54 mmol, 28%) as a yellow oil. MS (ESI) m/z 160.0 [M+H−Boc]⁺

Step 8. A solution of 2-(4-(tert-butoxycarbonyl)-1,4-oxazepan-7-yl)acetic acid (0.20 g, 0.77 mmol), 6-methyl-N1-(3-(9-(tetrahydro-2H-pyran-2-yl)-9H-purin-6-yl)pyridin-2-yl)benzene-1,3-diamine (0.31 g, 0.77 mmol), 1-[bis(dimethylamino)methylene]-1H-1,2,3-triazolo[4,5-b]pyridinium 3-oxid hexafluorophosphate (0.44 g, 1.16 mmol) and triethylamine (0.12 g, 1.16 mmol) in dichloromethane (25 mL) was stirred at room temperature for 16 hours. The reaction mixture was diluted with water (20 mL), extracted with dichloromethane (100 mL), washed with water (20 mL), dried over anhydrous sodium sulfate, filtered and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give tert-butyl-7-(2-(4-methyl-3-(3-(9-(tetrahydro-2H-pyran-2-yl)-9H-purin-6-yl)pyridin-2-ylamino)phenylamino)-2-oxoethyl)-1,4-oxazepane-4-carboxylate (60 mg, 0.09 mmol, 12%) as a white solid. MS (ESI) m/z 643.0 [M+H−Boc]⁺

Step 9. tert-Butyl-7-(2-(4-methyl-3-(3-(9-(tetrahydro-2H-pyran-2-yl)-9H-purin-6-yl)pyridin-2-ylamino)phenylamino)-2-oxoethyl)-1,4-oxazepane-4-carboxylate (60 mg, 0.09 mmol) was dissolved in trifluoroacetic acid (2 mL) and dichloromethane (5 mL) and the mixture was stirred at room temperature for 5 hours. The solvent was removed and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-2-(1,4-oxazepan-7-yl)acetamide (25 mg, 0.05 mmol, 59%) as a yellow solid. ¹H NMR (400 MHZ, DMSO-d₆) δ 12.49 (s, 1H), 9.83 (t, J=9.6 Hz, 2H), 8.96 (d, J=3.6 Hz, 1H), 8.56 (d, J=5.6 Hz, 1H), 8.46 (s, 1.0 Hz, 1H), 8.30 (s, 1H), 7.40 (d, J=8.4 Hz, 1H), 7.15 (d, J=8.4 Hz, 1H), 7.01 (d, J=8.0 Hz, 1H), 4.13-4.11 (m, 1H), 3.92-3.82 (m, 1H), 3.80-3.75 (m, 4H), 2.91-2.84 (m, 3H), 2.50-2.34 (m, 5H), 1.98-1.76 (m, 1H), 1.62-1.53 (m, 1H). MS (ESI) m/z 459.1 [M+H]⁺

### Example 86. Synthesis of N-(3-((3-(9H-purin-6-yl)pyridin-2-yl)amino)-4-methylphenyl)-2-(1,4-diazabicyclo[3.2.1]octan-4-yl)acetamide (Compound I-89)

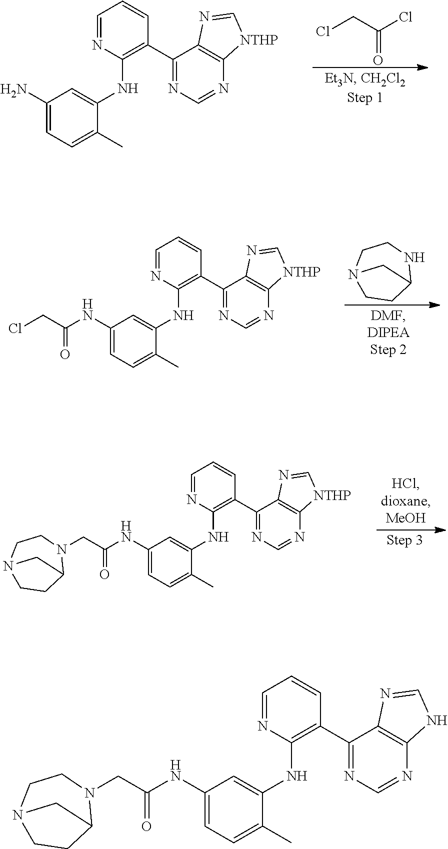

Step 1. To a solution of 6-methyl-N1-(3-(9-(tetrahydro-2H-pyran-2-yl)-9H-purin-6-yl)pyridin-2-yl)benzene-1,3-diamine (100 mg, 0.25 mmol) in dichloromethane (3 mL) was added triethylamine (76 mg, 0.75 mmol) and 2-chloroacetyl chloride (28 mg, 0.25 mmol). The mixture was stirred at 0° C. to room temperature for 1 hour. The mixture was extracted with ethyl acetate (30 mL×3). The combined organic layer was washed with brine (30 mL), dried over anhydrous sodium sulfate, and filtered. The filtrate was concentrated under reduced pressure and purified by flash chromatography (ethyl acetate) to give 2-chloro-N-(4-methyl-3-((3-(9-(tetrahydro-2H-pyran-2-yl)-9H-purin-6-yl)pyridin-2-yl)amino)phenyl)acetamide (80 mg, 0.17 mmol, 73%) as a yellow solid. MS (ESI) m/z 478.0 [M+H]⁺

Step 2. To a solution of 1,4-diazabicyclo[3.2.1]octane (20 mg, 0.18 mmol) in N,N-dimethylformamide (3 mL) was added N,N-diisopropylethylamine (69 mg, 0.54 mmol) and 2-chloro-N-(4-methyl-3-((3-(9-(tetrahydro-2H-pyran-2-yl)-9H-purin-6-yl)pyridin-2-yl)amino)phenyl)acetamide (80 mg, 0.17 mmol). The mixture was stirred at room temperature for 16 hours. The mixture was extracted with ethyl acetate (30 mL×3). The combined organic layers were washed with brine (30 mL), dried over anhydrous sodium sulfate, and filtered. The filtrate was concentrated under reduced pressure. The residue was purified by flash chromatography (ethyl acetate) to give 2-(1,4-diazabicyclo[3.2.1]octan-4-yl)-N-(4-methyl-3-((3-(9-(tetrahydro-2H-pyran-2-yl)-9H-purin-6-yl)pyridin-2-yl)amino)phenyl)acetamide (80 mg, 0.14 mmol, 87%) as a yellow solid. MS (ESI) m/z 554.0 [M+H]⁺

Step 3. To a solution of 2-(1,4-diazabicyclo[3.2.1]octan-4-yl)-N-(4-methyl-3-((3-(9-(tetrahydro-2H-pyran-2-yl)-9H-purin-6-yl)pyridin-2-yl)amino)phenyl)acetamide (80 mg, 0.14 mmol) in methanol (4 mL) was added hydrochloric acid (4 M in dioxane, 4 mL) at 25° C., and the mixture was stirred at 25° C. for 1 hour. The reaction mixture was concentrated and diluted with methanol (10 mL), and the pH was adjusted to 8 with ammonia (7 N in methanol). The mixture concentrated under reduced pressure and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((3-(9H-purin-6-yl)pyridin-2-yl)amino)-4-methylphenyl)-2-(1,4-diazabicyclo[3.2.1]octan-4-yl)acetamide (10.5 mg, 0.022 mmol, 16%) as a yellow solid. ¹H NMR (400 MHZ, DMSO-d₆) δ 2.01 (s, 1H), 2.23-2.32 (m, 1H), 2.46 (s, 3H), 2.95-2.99 (m, 1H), 3.42-3.52 (m, 3H,), 3.61 (t, J=11.6 Hz, 3H), 3.74 (s, 1H), 4.07 (s, 1H), 4.40 (q, J=16.0 Hz, 2H), 6.99 (q, J=4.8 Hz, 1H), 7.18 (d, J=8.4 Hz, 1H), 7.34 (dd, J₁-2.0 Hz, J₂-8.0 Hz, 1H), 8.19 (s, 1H), 8.24 (dd, J_{1=1.6} Hz, J_{2=4.6} Hz, 1H), 8.62 (d, J=2.0 Hz, 1H), 8.72 (s, 1H), 10.02 (dd, J_{1=2.0} Hz, J_{2=7.8} Hz, 1H), 10.45 (s, 1H), 13.07 (s, 1H). MS (ESI) m/z 469.8 [M+H]⁺

### Example 87. Synthesis of N-(3-((3-(9H-purin-6-yl)pyridin-2-yl)amino)-4-methylphenyl)-4-(difluoromethyl)-5-methylpicolinamide (Compound IA-90)

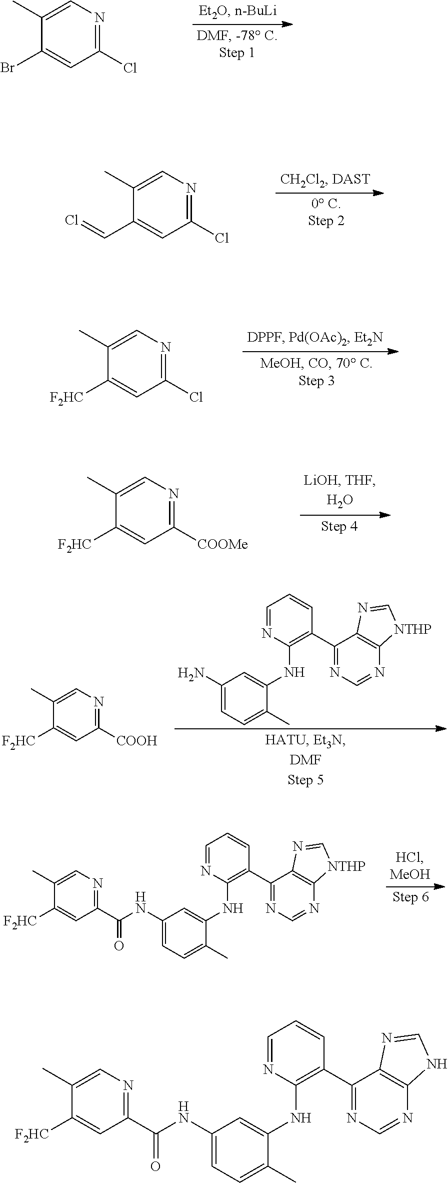

Step 1. To a solution of 4-bromo-2-chloro-5-methylpyridine (1 g, 4.85 mmol) in ethyl ether (5 mL) was added n-butyllithium (64 mg, 9.70 mmol) at −78° C. The mixture was stirred at −78° C. for 30 minutes, then N,N-dimethylformamide (2 mL) was added and stirred at −78° C. for 2 hours. The mixture was quenched with water (30 mL) and extracted with ethyl acetate (30 mL×3). The combined organic phases were dried over sodium sulphate, filtered and concentrated under reduced pressure to afford 2-chloro-5-methylisonicotinaldehyde (538 mg, 3.47 mmol, 72%) as a yellow oil. MS (ESI) m/z 156.3 [M+H]⁺

Step 2. To a solution of 2-chloro-5-methylisonicotinaldehyde (538 mg, 3.47 mmol) in dichloromethane (5 mL) was added diethylaminosulphur trifluoride (2.8 g, 17.35 mmol) at 0° C. The mixture was stirred at 0° C. for 16 hours. The mixture was quenched with water (20 mL) and extracted with ethyl acetate (10 mL×3). The combined organic phases were dried over sodium sulfate, filtered and concentrated under reduced pressure to afford 2-chloro-4-(difluoromethyl)-5-methylpyridine (378 mg, 2.13 mmol, 62%) as a yellow oil. MS (ESI) m/z 178.3 [M+H]⁺

Step 3. A mixture of 2-chloro-4-(difluoromethyl)-5-methylpyridine (338 mg, 1.91 mmol), 1, l′-bis(diphenylphosphino)ferrocene (212 mg, 0.382 mmol), palladium acetate (43 mg, 0.191 mmol) and triethylamine (0.5 mL) in methanol (5 mL) was stirred at 70° C. for 16 hours under an atmosphere of carbon monoxide gas. The reaction mixture was cooled to room temperature and concentrated. The crude residue was purified by flash chromatography (dichloromethane/methanol=10/1) to afford methyl 4-(difluoromethyl)-5-methylpicolinate (137 mg, 0.68 mmol, 36%) as a yellow solid. MS (ESI) m/z 201.9 [M+H]⁺

Step 4. A solution of methyl 4-(difluoromethyl)-5-methylpicolinate (137 mg, 0.68 mmol) and lithium hydroxide (33 mg, 1.36 mmol) in tetrahydrofuran (5 mL) and water (1 mL) was stirred at room temperature for 2 hours. The mixture was washed with water and extracted with ethyl acetate (30 mL×3). The combined organic phases were dried over sodium sulfate, filtered and concentrated under reduced pressure to afford 4-(difluoromethyl)-5-methylpicolinic acid (113 mg, 0.60 mmol, 89%) as a yellow solid. MS (ESI) m/z 188.2 [M+H]⁺

Step 5. A solution of 4-(difluoromethyl)-5-methylpicolinic acid (63 mg, 0.34 mmol), 6-methyl-N1-(3-(9-(tetrahydro-2H-pyran-2-yl)-9H-purin-6-yl)pyridin-2-yl)benzene-1,3-diamine (136 mg, 0.34 mmol), 1-[bis(dimethylamino)methylene]-1H-1,2,3-triazolo[4,5-b]pyridinium 3-oxid hexafluorophosphate (388 mg, 1.02 mmol) and triethylamine (0.2 mL) in N,N-dimethylformamide (3 mL) was stirred at room temperature for 2 hours. The mixture was washed with brine (30 mL) and extracted with ethyl acetate (10 mL×3). The combined organic phases were dried over sodium sulfate, filtered and concentrated under reduced pressure to afford 4-(difluoromethyl)-5-methyl-N-(4-methyl-3-((3-(9-(tetrahydro-2H-pyran-2-yl)-9H-purin-6-yl)pyridin-2-yl)amino)phenyl)picolinamide (103 mg, 0.18 mmol, 53%) as a yellow solid. MS (ESI) m/z 571.0 [M+H]⁺

Step 6. 4-(Difluoromethyl)-5-methyl-N-(4-methyl-3-((3-(9-(tetrahydro-2H-pyran-2-yl)-9H-purin-6-yl)pyridin-2-yl)amino)phenyl)picolinamide (103 mg, 0.18 mmol) in hydrochloric acid (3 M in methanol, 3 mL) was stirred at room temperature for 2 hours. The reaction mixture was concentrated, and then the pH was adjusted to alkaline by adding ammonia (7 M in methanol). The resulting mixture was purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-4-(difluoromethyl)-5-methylpicolinamide (11.4 mg, 0.02 mmol, 13%). ¹H NMR (400 MHZ, DMSO-d₆) δ 13.87 (s, 1H), 12.33 (s, 1H), 10.51 (s, 1H), 9.82 (s, 1H), 9.09 (s, 1H), 8.80 (d, J=1.7 Hz, 1H), 8.74 (d, J=5.7 Hz, 2H), 8.37 (dd, J=4.6, 1.9 Hz, 1H), 8.22 (s, 1H), 7.54 (dd, J=8.2, 2.0 Hz, 1H), 7.27 (dd, J=31.1, 22.8 Hz, 2H), 7.04 (dd, J=7.9, 4.7 Hz, 1H), 2.50 (s, 3H), 2.44 (s, 3H). MS (ESI) m/z 487.0 [M+H]⁺

### Example 88. Synthesis of N-(3-(3-(9H-purin-6-yl)pyridin-2-ylamino)-4-methylphenyl)-3-fluoro-5-methyl-4-(trifluoromethyl)picolinamide (Compound IA-91)

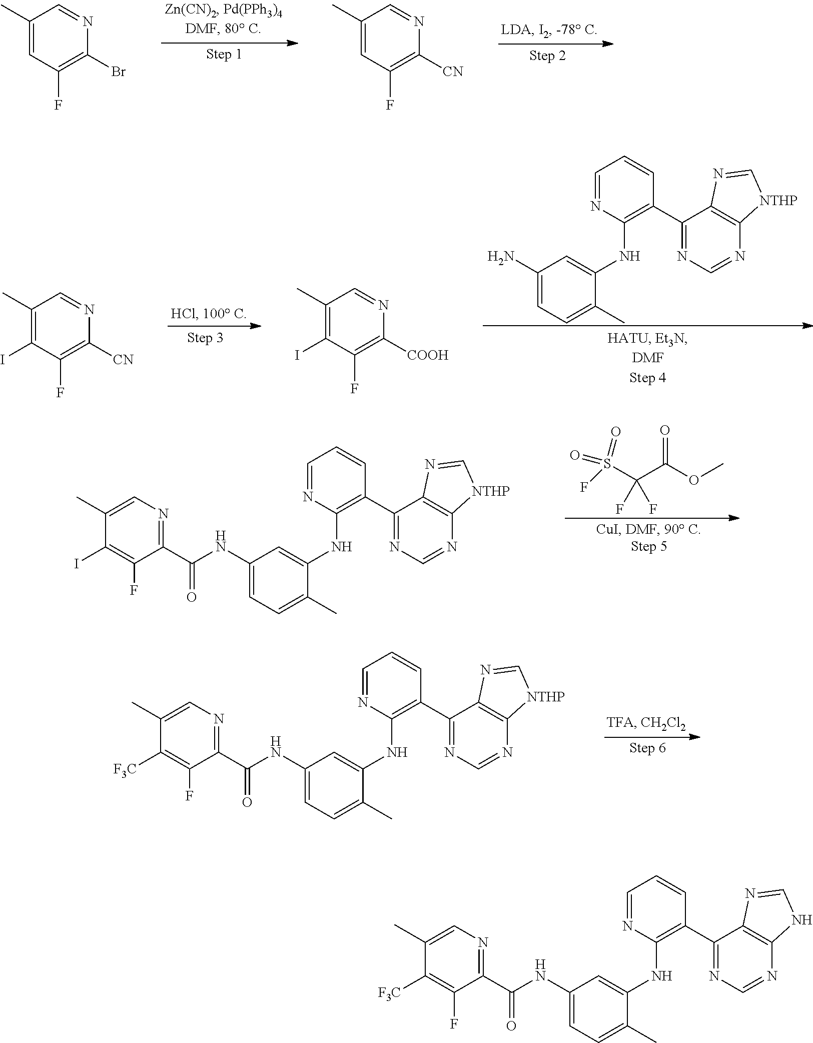

Step 1. A mixture of 2-bromo-3-fluoro-5-methylpyridine (1.9 g, 10.05 mmol), zinc cyanide (1.4 g, 12.06 mmol) and tetrakis(triphenylphosphine)palladium (1.16 g, 1.01 mmol) in N,N-dimethylformamide (10 mL) was stirred at 80° C. for 18 hours under nitrogen. The reaction mixture was cooled to room temperature, treated with water (100 mL) and extracted with ethyl acetate (50 mL×3). The combined organic layers were concentrated. The residue was purified by flash chromatography (petroleum ether/ethyl acetate=3/1) to afford 3-fluoro-5-methylpicolinonitrile (450 mg, 3.31 mmol, 33%) as a white solid. MS (ESI) m/z 137.0 [M+H]⁺

Step 2. To a solution of diisopropylamine (401 mg, 3.97 mmol) in tetrahydrofuran (30 mL) was added n-butyllithium (1.59 mL, 2.5 M in hexane). The mixture was stirred at −78° C. for 0.5 hour. 3-fluoro-5-methylpicolinonitrile (450 mg, 3.31 mmol) was added and the mixture was stirred at −78° C. for 0.5 hour. Iodine (1 g, 3.97 mmol) was added and the mixture was stirred at −78° C. for 1 hour. The reaction was treated with saturated sodium thiosulfate solution (20 mL) and extracted with ethyl acetate (50 mL×2). The combined organic layers were concentrated. The residue was purified by flash chromatography (petroleum ether/ethyl acetate=4/1) to afford 3-fluoro-4-iodo-5-methylpicolinonitrile (300 mg, 1.15 mmol, 35%) as a white solid. MS (ESI) m/z 263.0 [M+H]⁺

Step 3. A solution of 3-fluoro-4-iodo-5-methylpicolinonitrile (300 mg, 1.15 mmol) in hydrochloride (5 mL, 37%) was stirred at 100° C. for 18 hours. The reaction solution was cooled to room temperature, treated with water (20 mL) and extracted with ethyl acetate (30 mL×2). The combined organic layers were concentrated. The residue was purified by flash chromatography (petroleum ether/ethyl acetate=1/2) to afford 3-fluoro-4-iodo-5-methylpicolinic acid (220 mg, 0.78 mmol, 68%) as a white solid. MS (ESI) m/z 282.0 [M+H]⁺

Step 4. A mixture of 3-fluoro-4-iodo-5-methylpicolinic acid (220 mg, 0.78 mmol), 6-methyl-N1-(3-(9-(tetrahydro-2H-pyran-2-yl)-9H-purin-6-yl)pyridin-2-yl)benzene-1,3-diamine (314 mg, 0.78 mmol), 1-[bis(dimethylamino)methylene]-1H-1,2,3-triazolo[4,5-b]pyridinium 3-oxid hexafluorophosphate (357 mg, 0.94 mmol) and triethylamine (237 mg, 2.35 mmol) in N,N-dimethylformamide (5 mL) was stirred at room temperature for 2 hours. The reaction mixture was treated with water (50 mL). The solid was filtered, washed with water (3 mL) and dried to afford 3-fluoro-4-iodo-5-methyl-N-(4-methyl-3-((3-(9-(tetrahydro-2H-pyran-2-yl)-9H-purin-6-yl)pyridin-2-yl)amino)phenyl)picolinamide (480 mg, 0.72 mmol, 93%) as a yellow solid. MS (ESI) m/z 665.0 [M+H]⁺

Step 5. The mixture of 3-fluoro-4-iodo-5-methyl-N-(4-methyl-3-((3-(9-(tetrahydro-2H-pyran-2-yl)-9H-purin-6-yl)pyridin-2-yl)amino)phenyl)picolinamide (120 mg, 0.18 mmol), methyl 2,2-difluoro-2-(fluorosulfonyl)acetate (52 mg, 0.27 mmol) and cuprous iodide (3 mg, 0.02 mmol) in N,N-dimethylformamide (2 mL) was stirred at 90° C. for 2 hours under nitrogen. The reaction mixture was cooled to room temperature, treated with water (30 mL) and extracted with ethyl acetate (30 mL×2). The combined organic layers were concentrated. The residue was purified by flash chromatography (petroleum ether/ethyl acetate=2/3) to afford 3-fluoro-5-methyl-N-(4-methyl-3-((3-(9-(tetrahydro-2H-pyran-2-yl)-9H-purin-6-yl)pyridin-2-yl)amino)phenyl)-4-(trifluoromethyl)picolinamide (100 mg, 0.165 mmol, 92%) as a yellow solid. MS (ESI) m/z 607.1 [M+H]⁺

Step 6. To a solution of 3-fluoro-5-methyl-N-(4-methyl-3-((3-(9-(tetrahydro-2H-pyran-2-yl)-9H-purin-6-yl)pyridin-2-yl)amino)phenyl)-4-(trifluoromethyl)picolinamide (80 mg, 0.13 mmol) in dichloromethane (1 mL) was added trifluoroacetic acid (1 mL) at room temperature. After stirring at room temperature for 1 hour, the reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-3-fluoro-5-methyl-4-(trifluoromethyl)picolinamide (19.8 mg, 0.038 mmol, 29%) as a yellow solid. ¹H NMR (400 MHZ, DMSO-d₆) δ 13.88 (s, 1H), 12.36 (s, 1H), 10.60 (s, 1H), 9.82 (s, 1H), 9.09 (s, 1H), 8.73 (s, 1H), 8.68 (s, 2H), 8.36 (dd, J=4.7, 1.8 Hz, 1H), 7.56-7.43 (m, 1H), 7.25 (d, J=8.3 Hz, 1H), 7.05 (dd, J=7.9, 4.7 Hz, 1H), 2.58 (d, J=2.7 Hz, 3H), 2.45 (s, 3H). MS (ESI) m/z 523.0 [M+H]⁺

### Example 89. Synthesis of N-(3-(3-(9H-purin-6-yl)pyridin-2-ylamino)-4-methylphenyl)-5-ethyl-4-(trifluoromethyl)picolinamide (Compound IA-92)

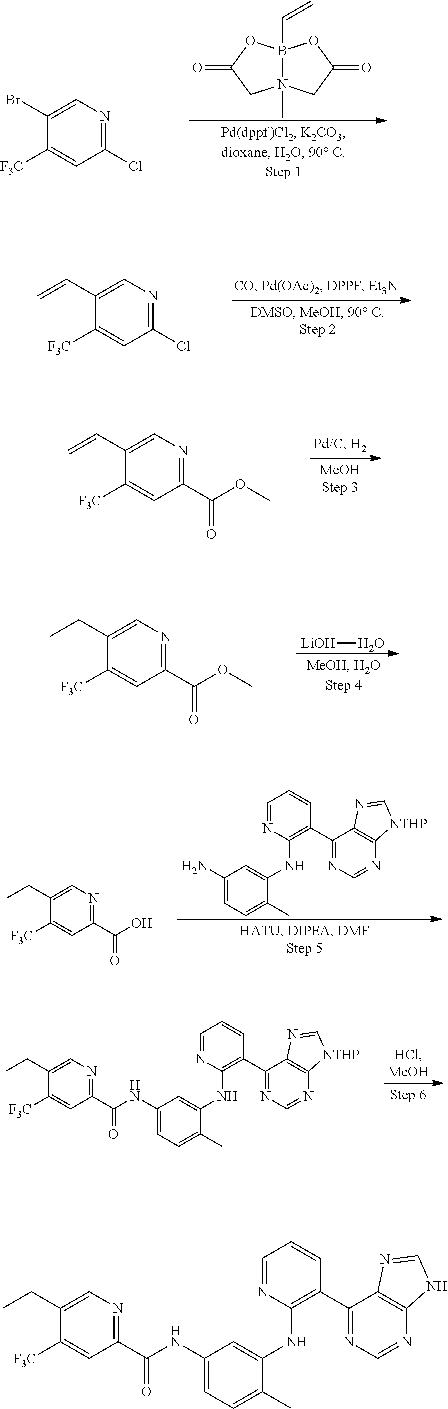

Step 1. A solution of 5-bromo-2-chloro-4-(trifluoromethyl)pyridine (2.2 g, 8.46 mmol), 6-methyl-2-vinyl-1,3,6,2-dioxazaborocane-4,8-dione (1.7 g, 9.3 mmol), 1,1′-bis(diphenylphosphino)ferrocene-palladium(II) dichloride dichloromethane complex (690 mg, 0.85 mmol) and potassium carbonate (2.33 g, 16.92 mmol) in 1,4-dioxane (20 mL) and water (5 mL) was stirred at 90° C. under nitrogen atmosphere for 16 hours. The mixture was cooled to room temperature and concentrated. The crude residue was purified flash chromatography (petroleum ether/ethyl acetate=3/1) to afford 2-chloro-4-(trifluoromethyl)-5-vinylpyridine (0.623 g, 3.0 mmol, 36%) as a yellow oil. MS (ESI) m/z 208.1 [M+H]⁺

Step 2. A mixture of 2-chloro-4-(trifluoromethyl)-5-vinylpyridine (623 mg, 3.0 mmol), 1,1′-bis (diphenylphosphino)ferrocene (664 mg, 1.2 mmol), palladium (II) acetate (135 mg, 0.6 mmol) and triethylamine (909 mg, 9.0 mmol) in dimethyl sulfoxide (5 mL) and methanol (3 mL) was stirred at 90° C. for 16 hours under carbon monoxide. After cooling to room temperature, the mixture was diluted with water (30 mL) and extracted with ethyl acetate (50 mL×2). The combined organic layers were washed with brine (30 mL×3), dried over anhydrous sodium sulfate, filtered, and concentrated under reduced pressure. The residue was purified by flash chromatography (ethyl acetate/petroleum ether=1/3) to afford methyl 4-(trifluoromethyl)-5-vinylpicolinate (115 mg, 0.49 mmol, 17%) as a brown solid. MS (ESI) m/z 232.1[M+H]⁺

Step 3. A mixture of methyl 4-(trifluoromethyl)-5-vinylpicolinate (115 mg, 0.49 mmol) and 10% palladium carbon catalyst (50 mg) in methanol (10 mL) was stirred at room temperature for 1 hour under hydrogen. The reaction mixture was filtered and the filtrate was concentrated. The crude product was purified by flash chromatography (ethyl acetate/petroleum ether=1/3) to afford methyl 5-ethyl-4-(trifluoromethyl)picolinate (110 mg, 0.47 mmol, 95%) as a brown oil. MS (ESI) m/z 234.1 [M+H]⁺

Step 4. To a solution of methyl 5-ethyl-4-(trifluoromethyl)picolinate (88 mg, 0.37 mmol) in methanol (5 mL) were added lithium hydroxide hydrate (78 mg, 1.85 mmol) and water (1 mL) at room temperature. The mixture was stirred at room temperature for 16 hours. The solvent was evaporated under reduced pressure. The residue was diluted with water (5 mL) and adjusted to pH 3 through the slow addition of 6 N hydrogen chloride. The resulting white precipitate was collected by filtration, washed with water (10 mL) and dried to afford 5-ethyl-4-(trifluoromethyl)picolinic acid (50 mg, 0.23 mmol, 61%) as a white solid. The crude product was used without further purification. MS (ESI) m/z 219.9 [M+H]⁺

Step 5. To a solution of 5-ethyl-4-(trifluoromethyl)picolinic acid (50 mg, 0.23 mmol) in N,N-dimethylformamide (3 mL) were added 6-methyl-N1-(3-(9-(tetrahydro-2H-pyran-2-yl)-9H-purin-6-yl)pyridin-2-yl)benzene-1,3-diamine (73 mg, 0.18 mmol), 1-[bis(dimethylamino)-methylene]-1H-1,2,3-triazolo[4,5-b]pyridinium 3-oxid hexafluorophosphate (96 mg, 0.25 mmol) and N,N-diisopropylethylamine (59 mg, 0.46 mmol) at room temperature. The reaction mixture was stirred room temperature for 2 hours. The reaction mixture was diluted with water (20 mL). The solid was collected by filtration and dried to afford 5-ethyl-N-(4-methyl-3-((3-(9-(tetrahydro-2H-pyran-2-yl)-9H-purin-6-yl)pyridin-2-yl)amino)phenyl)-4-(trifluoromethyl)-picolinamide (110 mg, 0.18 mmol, 80%) as a yellow solid. The crude product was used without further purification. MS (ESI) m/z 602.7 [M+H]⁺

Step 6. A solution of 5-ethyl-N-(4-methyl-3-((3-(9-(tetrahydro-2H-pyran-2-yl)-9H-purin-6-yl)pyridin-2-yl)amino)phenyl)-4-(trifluoromethyl)picolinamide (40 mg, 0.066 mmol) in methanolic hydrochloric acid (3 M, 4 mL) was stirred at room temperature for 2 hours. The mixture was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-5-ethyl-4-(trifluoromethyl)picolinamide (3.3 mg, 0.006 mmol, 9.7%) as a yellow solid. ¹H NMR (500 MHZ, DMSO-d₆) δ 13.86 (s, 1H), 12.33 (s, 1H), 10.56 (s, 1H), 9.84-9.82 (m, 1H), 9.08 (d, J=5.5 Hz, 1H), 8.93 (d, J=5.5 Hz, 1H), 8.80 (s, 1H), 8.72 (s, 1H), 8.36 (dd, J=5.5 Hz, 2.0 Hz, 1H), 8.26 (s, 1H), 7.54 (dd, J=8.5 Hz, 2.0 Hz, 1H), 7.25 (d, J=9.0 Hz, 1H), 7.04 (dd, J=8.0 Hz, 5.0 Hz, 1H), 2.93 (q, J=7.0 Hz, 2H), 2.44 (s, 3H), 1.29 (t, J=7.0 Hz, 3H). MS (ESI)m m/z 519.3 [M+H]⁺

### Example 90. Synthesis of N-(3-((3-(9H-purin-6-yl)pyridin-2-yl)amino)-4-methylphenyl)-6-methyl-4-(trifluoromethyl)picolinamide (Compound IA-93)

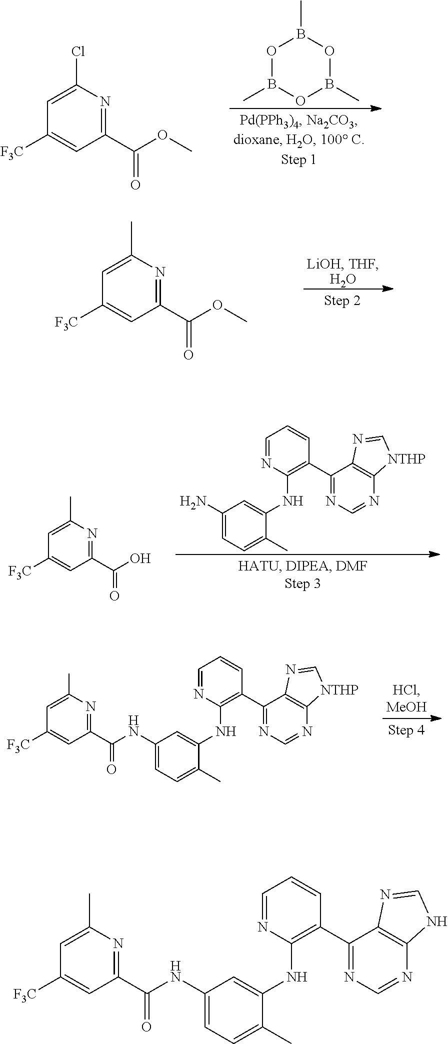

Step 1. To a mixture of methyl 6-chloro-4-(trifluoromethyl)picolinate (100 mg, 0.42 mmol) in 1,4-dioxane (3 mL) and water (1 mL) was added 2,4,6-trimethyl-1,3,5,2,4,6-trioxatriborinane (126 mg, 1.26 mmol), tetrakis(triphenylphosphine)palladium (43 mg, 0.041 mmol) and sodium carbonate (134 mg, 1.26 mmol). The reaction mixture was stirred at 100° C. for 16 hours under nitrogen. After cooling to room temperature, the mixture was diluted with water (20 mL) and extracted with ethyl acetate (20 mL×3). The combined organic layers were dried over sodium sulfate, filtered and concentrated under reduced pressure. The residue was purified by flash chromatography (petroleum ether/ethyl acetate=10/1) to afford methyl 6-methyl-4-(trifluoromethyl)picolinate (60 mg, 0.27 mmol, 65%) as a yellow oil. MS (ESI) m/z 220.1 [M+H]⁺

Step 2. To a solution of methyl 6-methyl-4-(trifluoromethyl)picolinate (60 mg, 0.27 mmol) in tetrahydrofuran (0.5 mL) were added lithium hydroxide hydrate (32 mg, 1.35 mmol) and water (0.5 mL) at room temperature. The mixture was stirred at room temperature for 1 hour. The solvent was removed under reduced pressure, and the residue was diluted with water (2 mL). The mixture was adjusted to pH 3 by adding 1 N hydrochloric acid slowly. The mixture was extracted with ethyl acetate (2 mL×3). The combined organic layers were dried over anhydrous sodium sulfate and filtered. The filtrate was concentrated under reduced pressure to afford 6-methyl-4-(trifluoromethyl)picolinic acid (50 mg, 0.24 mmol, 90%) as a colorless oil, which was used without further purification. MS (ESI) m/z 206.1 [M+H]⁺

Step 3. To a solution of 6-methyl-4-(trifluoromethyl)picolinic acid (50 mg, 0.24 mmol) in N,N-dimethylformamide (1 mL) was added 1-[bis(dimethylamino)methylene]-1H-1,2,3-triazolo[4,5-b]pyridinium 3-oxid hexafluorophosphate (110 mg, 0.29 mmol) and N,N-diisopropylethylamine (46 mg, 0.36 mmol) at room temperature. The solution was stirred at room temperature for 5 mins, then 6-methyl-N1-(3-(9-(tetrahydro-2H-pyran-2-yl)-9H-purin-6-yl)pyridin-2-yl)benzene-1,3-diamine (96 mg, 0.24 mmol) was added. The solution was stirred at room temperature for 2 hours. The mixture was diluted with water (20 mL) and extracted with ethyl acetate (20 mL×3). The combined organic phases were concentrated and the residue was purified by flash chromatography (petroleum ether/ethyl acetate=1/1) to afford 6-methyl-N-(4-methyl-3-((3-(9-(tetrahydro-2H-pyran-2-yl)-9H-purin-6-yl)pyridin-2-yl)amino)phenyl)-4-(trifluoromethyl)picolinamide (30 mg, 0.05 mmol, 21%) as a yellow solid. MS (ESI) m/z 589.2 [M+H]⁺

Step 4. A mixture of 6-methyl-N-(4-methyl-3-((3-(9-(tetrahydro-2H-pyran-2-yl)-9H-purin-6-yl)pyridin-2-yl)amino)phenyl)-4-(trifluoromethyl)picolinamide (30 mg, 0.05 mmol) in methanolic hydrochloric acid (3 M, 1 mL) was stirred at room temperature for 1 hour. The solvent was removed under reduced pressure and the residue was treated with ammonia (1 mL, 8 M in methanol). The solid was collected by filtration and dried under reduced pressure to afford N-(3-((3-(9H-purin-6-yl)pyridin-2-yl)amino)-4-methylphenyl)-6-methyl-4-(trifluoromethyl)picolinamide (11.1 mg, 0.02 mmol, 44%) as a yellow solid. ¹H NMR (500 MHZ, DMSO-d₆) δ 13.69 (s, 1H), 12.38 (s, 1H), 10.44 (s, 1H), 9.81 (s, 1H), 9.06 (s, 1H), 8.79 (s, 1H), 8.69 (s, 1H), 8.37 (s, 1H), 8.14 (s, 1H), 7.97 (s, 1H), 7.53 (d, J=6.8 Hz, 1H), 7.27 (d, J=7.2 Hz, 1H), 7.04 (s, 1H), 2.76 (s, 3H), 2.45 (s, 3H). MS (ESI) m/z 505.0 [M+H]⁺

### Example 91. Synthesis of N-(3-(3-(9H-purin-6-yl)pyridin-2-ylamino)-4-methylphenyl)-5-(1-methylazetidin-3-yloxy)-4-(trifluoromethyl)picolinamide (Compound IA-94)

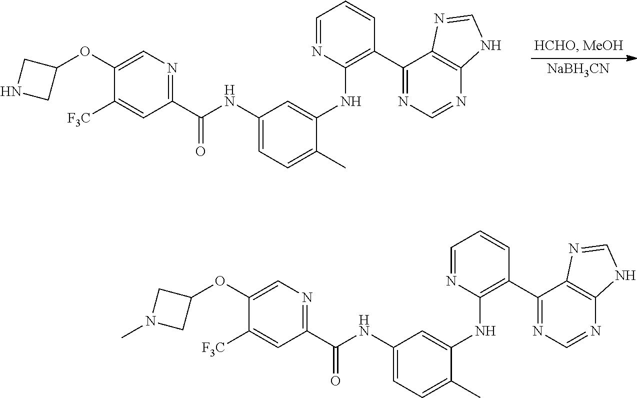 [SMILES: Cc1ccc(NC(=O)c2cc(C(F)(F)F)c(OC3CNC3)cn2)cc1Nc1ncccc1-c1ncnc2[nH]cnc12 ; "NaBH3CN" ; Cc1ccc(NC(=O)c2cc(C(F)(F)F)c(OC3CN(C)C3)cn2)cc1Nc1ncccc1-c1ncnc2[nH]cnc12]

A solution of N-(3-(3-(9H-purin-6-yl)pyridin-2-ylamino)-4-methylphenyl)-5-(azetidin-3-yloxy)-4-(trifluoromethyl)picolinamide (13 mg, 0.023 mmol) and formaldehyde (2 mg, 0.023 mmol) in methanol (5 mL) was stirred at room temperature for 2 hours. Sodium cyanoborohydride (3 mg, 0.046 mmol) was added and the reaction mixture was stirred at room temperature for 2 hours. The reaction mixture was concentrated and purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-(3-(9H-purin-6-yl)pyridin-2-ylamino)-4-methylphenyl)-5-(1-methylazetidin-3-yloxy)-4-(trifluoromethyl)picolinamide (6.7 mg, 0.012 mmol, 33.5%) as a yellow solid. ¹H NMR (500 MHZ, DMSO-d₆) δ 12.31 (s, 1H), 10.40 (s, 1H), 9.81 (d, J=4.5 Hz, 1H), 9.08 (s, 1H), 8.77 (d, J=1.5 Hz, 1H), 8.72 (s, 1H), 8.52 (s, 1H), 8.36 (q, J=2.0, 1H), 8.26 (s, 1H), 7.51 (dd, J=8.0 Hz, 2.0 Hz, 1H), 7.24 (d, J=8.5 Hz, 1H), 7.04 (dd, J=8.0 Hz, 5.0 Hz, 1H), 5.28 (m, 1H), 3.84 (t, J=7.5 Hz, 2H), 3.10 (t, J=5.5 Hz, 2H), 2.44 (s, 3H), 2.33 (s, 3H). MS (ESI) m/z 575.7 [M+H]⁺

### Example 92. Synthesis of N-(3-(3-(9H-purin-6-yl)pyridin-2-ylamino)-4-methylphenyl)-5-(oxetan-3-yloxy)-4-(trifluoromethyl)picolinamide (Compound IA-95)

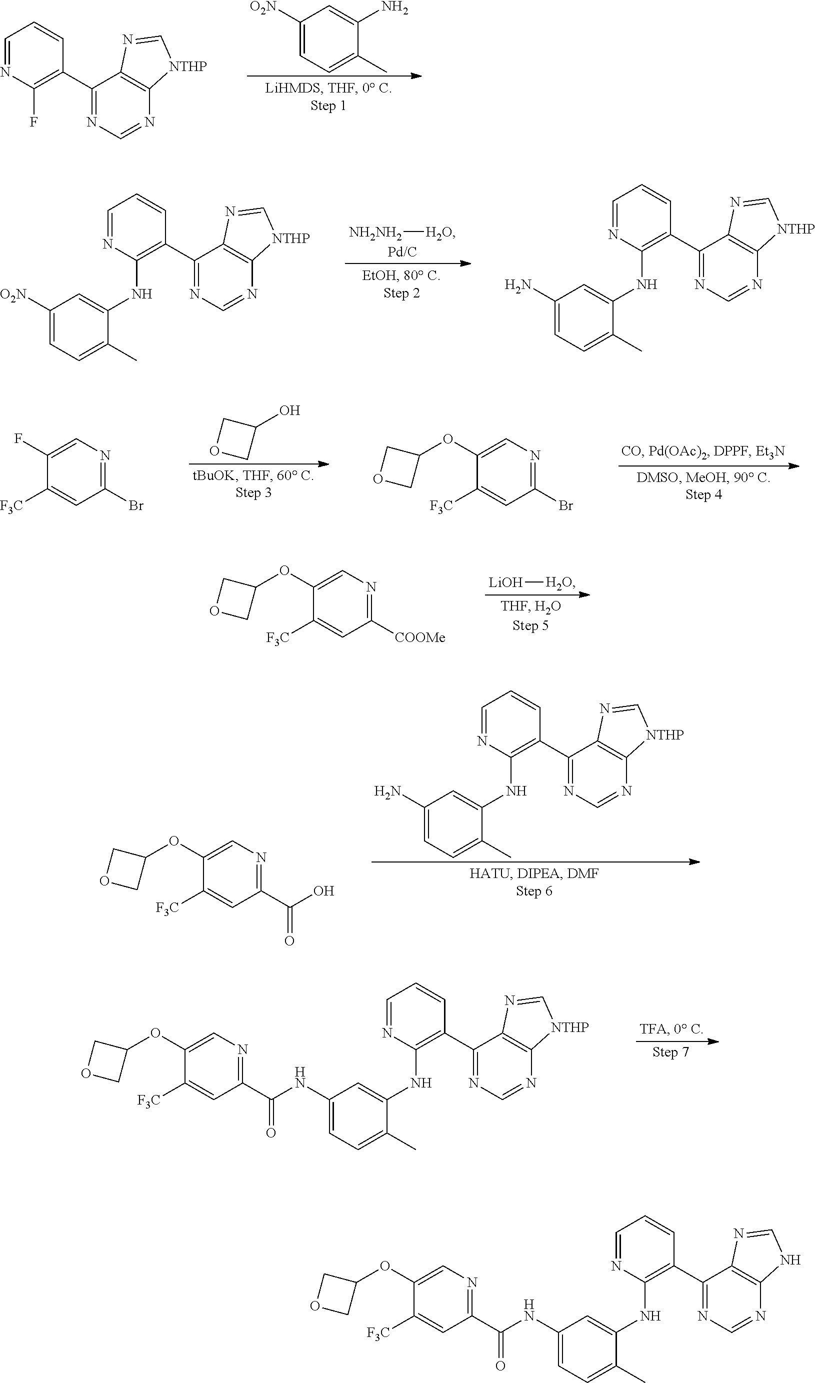

Step 1. To a solution of 2-methyl-5-nitroaniline (2 g, 13.1 mmol) and 6-(2-fluoropyridin-3-yl)-9-(tetrahydro-2H-pyran-2-yl)-9H-purine (3.92 g, 13.1 mmol) in tetrahydrofuran (80 mL) was added lithium hexamethyldisilazide (24.6 mL, 39.3 mmol, 1.6 M in tetrahydrofuran) at 0° C. and stirred at 0° C. for 1 hour. The reaction mixture was quenched with a saturated solution of ammonium chloride (40 mL), extracted with dichloromethane (100 mL×2), washed with brine (100 mL), dried over sodium sulfate and concentrated to afford N-(2-methyl-5-nitrophenyl)-3-(9-(tetrahydro-2H-pyran-2-yl)-9H-purin-6-yl)pyridin-2-amine (6.4 g) as a yellow solid. MS (ESI) m/z 432.3 [M+H]⁺

Step 2. To a solution of N-(2-methyl-5-nitrophenyl)-3-(9-(tetrahydro-2H-pyran-2-yl)-9H-purin-6-yl)pyridin-2-amine (6.4 g, 13.1 mmol) in ethanol (150 mL) was added hydrazine hydrate (23 mL) and 10% palladium on carbon (2.3 g). The reaction mixture was stirred at 80° C. for 1 hour, filtered through celite. and concentrated. The residue was diluted with dichloromethane (100 mL), washed with brine (100 mL), dried over sodium sulfate and concentrated. The residue was purified by flash chromatography (dichloromethane/methanol=90/1) to afford 6-methyl-N1-(3-(9-(tetrahydro-2H-pyran-2-yl)-9H-purin-6-yl)pyridin-2-yl)benzene-1,3-diamine (3 g, 7.5 mmol, 57%) as a yellow solid. MS (ESI) m/z 401.9 [M+H]⁺

Step 3. To a solution of 2-bromo-5-fluoro-4-(trifluoromethyl)pyridine (317 mg, 1.3 mmol) and oxetan-3-ol (106 mg, 1.43 mmol) in tetrahydrofuran (9 mL) was added potassium tert-butoxide (1.43 mL, 1.43 mmol, 1 M in tetrahydrofuran). The reaction mixture was stirred at 60° C. for 3 hours and then concentrated. The residue was purified by flash chromatography (petroleum ether/ethyl acetate=4/1) to afford 2-bromo-5-(oxetan-3-yloxy)-4-(trifluoromethyl)pyridine (340 mg, 1.14 mmol, 88%) as a white solid. MS (ESI) m/z 297.6 [M+H]⁺

Step 4. A mixture of 2-bromo-5-(oxetan-3-yloxy)-4-(trifluoromethyl)pyridine (320 mg, 1.07 mmol), 1, l′-bis(diphenylphosphino)ferrocene (237 mg, 0.42 mmol), palladium (II) acetate (48 mg, 0.21 mmol) and triethylamine (432 mg, 4.28 mmol) in dimethyl sulfoxide (24 mL) and methanol (15 mL) was stirred at 90° C. for 16 hours under carbon monoxide. The reaction mixture was cooled to room temperature, quenched with water (80 mL) and extracted with ethyl acetate (50 mL×2). The combined organic layers were washed with brine (30 mL×3), dried over anhydrous sodium sulfate, filtered and concentrated under reduced pressure. The residue was purified by flash chromatography (ethyl acetate in petroleum ether from 30% to 50%) to afford methyl 5-(oxetan-3-yloxy)-4-(trifluoromethyl)picolinate (260 mg, 0.94 mmol, 88%) as a yellow solid. MS (ESI) m/z 278.1 [M+H]⁺

Step 5. To a solution of methyl 5-(oxetan-3-yloxy)-4-(trifluoromethyl)picolinate (240 mg, 0.87 mmol) in tetrahydrofuran (3.6 mL) were added lithium hydroxide hydrate (182 mg, 4.33 mmol) and water (3.6 mL). The mixture was stirred at 25° C. for 1.5 hours. The solvent was evaporated under reduced pressure and the residue was diluted with water (5 mL). The mixture was adjusted to pH 3 by adding 1 N hydrochloric acid slowly at 0° C. The solid was filtered and dried to afford 5-(oxetan-3-yloxy)-4-(trifluoromethyl)picolinic acid (170 mg, 0.65 mmol, 74%) as a white solid. MS (ESI) m/z 264.1 [M+H]⁺

Step 6. A solution of 6-methyl-N1-(3-(9-(tetrahydro-2H-pyran-2-yl)-9H-purin-6-yl)pyridin-2-yl)benzene-1,3-diamine (72 mg, 0.18 mmol), 5-(oxetan-3-yloxy)-4-(trifluoromethyl)picolinic acid (52 mg, 0.2 mmol), 1-[bis(dimethylamino)methylene]-1H-1,2,3-triazolo[4,5-b]pyridinium 3-oxid hexafluorophosphate (114 mg, 0.3 mmol) and N,N-diisopropylethylamine (77 mg, 0.6 mmol) in N,N-dimethylformamide (1 mL) was stirred at 25° C. for 1 hour. The mixture was diluted with ethyl acetate (60 mL), washed with water (50 mL) and brine (40 mL×3), dried over sodium sulfate, filtered and concentrated. The residue was purified by flash chromatography (ethyl acetate/petroleum ether=3/5) to afford N-(4-methyl-3-((3-(9-(tetrahydro-2H-pyran-2-yl)-9H-purin-6-yl)pyridin-2-yl)amino)phenyl)-5-(oxetan-3-yloxy)-4-(trifluoromethyl)picolinamide (100 mg, 0.15 mmol, 86%) as a yellow solid. MS (ESI) m/z 646.7 [M+H]⁺

Step 7. A solution of N-(4-methyl-3-((3-(9-(tetrahydro-2H-pyran-2-yl)-9H-purin-6-yl)pyridin-2-yl)amino)phenyl)-5-(oxetan-3-yloxy)-4-(trifluoromethyl)picolinamide (40 mg, 0.062 mmol) in trifluoroacetic acid (1 mL) was stirred at 0° C. for 1 hour. The reaction mixture was concentrated under reduced pressure and purified by prep-HPLC (Column: BOSTON pHlex ODS 10 μm, 21.2×250 mm, 120 Å; Mobile phase: acetonitrile/0.1% ammonium bicarbonate) to afford N-(3-((3-(9H-purin-6-yl)pyridin-2-yl)amino)-4-methylphenyl)-5-(oxetan-3-yloxy)-4-(trifluoromethyl)picolinamide (11.6 mg, 0.02 mmol, 33%) as a yellow solid. ¹H NMR (500 MHZ, DMSO-d₆) δ 12.32 (s, 1H), 10.39 (s, 1H), 9.78 (d, J=7.3 Hz, 1H), 9.07 (s, 1H), 8.78 (d, J=1.6 Hz, 1H), 8.71 (s, 1H), 8.40 (s, 1H), 8.36 (dd, J=4.5, 1.7 Hz, 1H), 8.28 (s, 1H), 7.55-7.47 (m, 1H), 7.24 (d, J=8.3 Hz, 1H), 7.04 (dd, J=7.8, 4.7 Hz, 1H), 5.88-5.68 (m, 1H), 5.04 (t, J=6.8 Hz, 2H), 4.64 (dd, J=7.6, 4.8 Hz, 2H), 2.44 (s, 3H). MS (ESI) m/z 562.7 [M+H]⁺

### Example 93. N-(3-(3-(9H-purin-6-yl)pyridin-2-ylamino)-4-methylphenyl)-5-(azetidin-3-yloxy)-4-(trifluoromethyl)picolinamide (Compound IA-96)

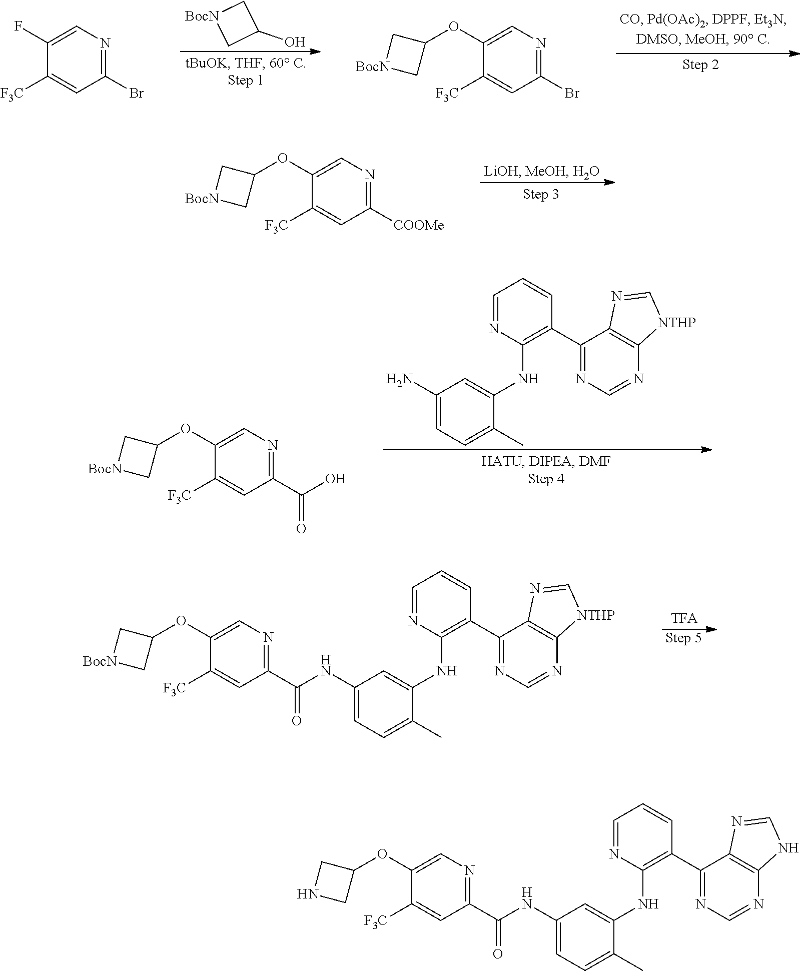

Step 1. The mixture of 2-bromo-5-fluoro-4-(trifluoromethyl)pyridine (300 mg, 1.23 mmol), tert-butyl 3-hydroxyazetidine-1-carboxylate (256 mg, 1.48 mmol) and potassium tert-butoxide (165 mg, 1.48 mmol) in tetrahydrofuran (4 mL) was stirred at 60° C. for 16 hours. The reaction mixture was concentrated and the residue was purified by flash chromatography (ethyl acetate/petroleum ether=1/1) to deliver tert-butyl 3-((6-bromo-4-(trifluoromethyl)pyridin-3-yl)oxy)azetidine-1-carboxylate (500 mg, 1.26 mmol, 99%) as a white solid. MS (ESI) m/z 343.1 [M−56+H]⁺

Step 2. A mixture of tert-butyl 3-((6-bromo-4-(trifluoromethyl)pyridin-3-yl)oxy)azetidine-1-carboxylate (300 mg, 0.76 mmol), 1, l′-bis (diphenylphosphino)ferrocene (166 mg, 0.30 mmol), palladium (II) acetate (33 mg, 0.15 mmol) and triethylamine (303 mg, 3.0 mmol) in dimethyl sulfoxide (5 mL) and methanol (2 mL) was stirred at 90° C. for 16 hours under carbon monoxide. The reaction mixture was cooled to room temperature, then quenched with water (80 mL) and extracted with ethyl acetate (50 mL×2). The combined organic layers were washed with brine (30 mL×3), dried over anhydrous sodium sulfate, filtered and concentrated under reduced pressure. The residue was purified by flash chromatography (ethyl acetate/petroleum ether=1/1) to afford methyl 5-((1-(tert-butoxycarbonyl)azetidin-3-yl)oxy)-4-(trifluoromethyl)picolinate (180 mg, 0.48 mmol, 63%) as a yellow oil. MS (ESI) m/z 377.0 [M+H]⁺

Step 3. To a solution of methyl 5-((1-(tert-butoxycarbonyl)azetidin-3-yl)oxy)-4-(trifluoromethyl)picolinate (180 mg, 0.48 mmol) in methanol (3 mL) were added lithium hydroxide hydrate (100 mg, 2.39 mmol) and water (1 mL) at room temperature. The mixture was stirred at room temperature for 3 hours. The solvent was evaporated under reduced pressure and the residue was diluted with water (10 mL). The mixture was adjusted to pH 3 by adding 6 N hydrochloric acid slowly. The resulting white precipitate was collected by filtration, washed with water (10 mL) and dried to afford 5-((1-(tert-butoxycarbonyl)azetidin-3-yl)oxy)-4-(trifluoromethyl)picolinic acid (105 mg, 0.29 mmol, 60.7%) as a white solid. MS (ESI) m/z 363.0 [M+H]⁺

Step 4. A solution of 5-((1-(tert-butoxycarbonyl)azetidin-3-yl)oxy)-4-(trifluoromethyl) picolinic acid (50 mg, 0.14 mmol), 6-methyl-N1-(3-(9-(tetrahydro-2H-pyran-2-yl)-9H-purin-6-yl)pyridin-2-yl)benzene-1,3-diamine (56 mg, 0.14 mmol), 1-[bis(dimethylamino)methylene]-1H-1,2,3-triazolo[4,5-b]pyridinium 3-oxid hexafluorophosphate (58 mg, 0.15 mmol) and N,N-diisopropylethylamine (36 mg, 0.28 mmol) in N,N-dimethylformamide (3 mL) was stirred at room temperature for 2 hours. The reaction solution was quenched with water (10 mL). The solid was collected by filtration and dried to afford tert-butyl 3-((6-((4-methyl-3-((3-(9-(tetrahydro-2H-pyran-2-yl)-9H-purin-6-yl)pyridin-2-yl)amino)phenyl)carbamoyl)-4-(trifluoromethyl)pyridin-3-yl)oxy)azetidine-1-carboxylate (100 mg, 0.13 mmol, 96%) as a yellow solid. MS (ESI) m/z 746.3 [M+H]⁺

Step 5. A solution of tert-butyl 3-((6-((4-methyl-3-((3-(9-(tetrahydro-2H-pyran-2-yl)-9H-purin-6-yl)pyridin-2-yl)amino)phenyl)carbamoyl)-4-(trifluoromethyl)pyridin-3-yl)oxy)azetidine-1-carboxylate (50 mg, 0.067 mmol) in trifluoroacetic acid (2 mL) was stirred at room temperature for 2 hours. The reaction mixture was concentrated and the residue was purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-(3-(9H-purin-6-yl)pyridin-2-ylamino)-4-methylphenyl)-5-(azetidin-3-yloxy)-4-(trifluoromethyl)-picolinamide (19.3 mg, 0.034 mmol, 51.5%) as a yellow solid. ¹H NMR (500 MHZ, DMSO-d₆) δ 12.33 (s, 1H), 10.40 (s, 1H), 9.79 (d, J=7.5 Hz, 1H), 9.07 (s, 1H), 8.78 (d, J=2.0 Hz, 1H), 8.70 (s, 1H), 8.47 (s, 1H), 8.36 (t, J=3.5, 1H), 8.26 (s, 1H), 7.51 (dd, J=8.5 Hz, 2.0 Hz, 1H), 7.24 (d, J=8.5 Hz, 1H), 7.04 (dd, J=7.5, 4.5 Hz, 1H), 5.49 (t, J=6.5 Hz, 1H), 3.98-3.95 (m, 2H), 3.68-3.65 (m, 2H), 2.36 (s, 3H). MS (ESI) m/z 562.1 [M+H]⁺

### Example 94. Synthesis of N-(3-(3-(9H-purin-6-yl)pyridin-2-ylamino)-4-methylphenyl)-5-(2-(dimethylamino)ethoxy)-4-(trifluoromethyl)picolinamide (Compound IA-97)

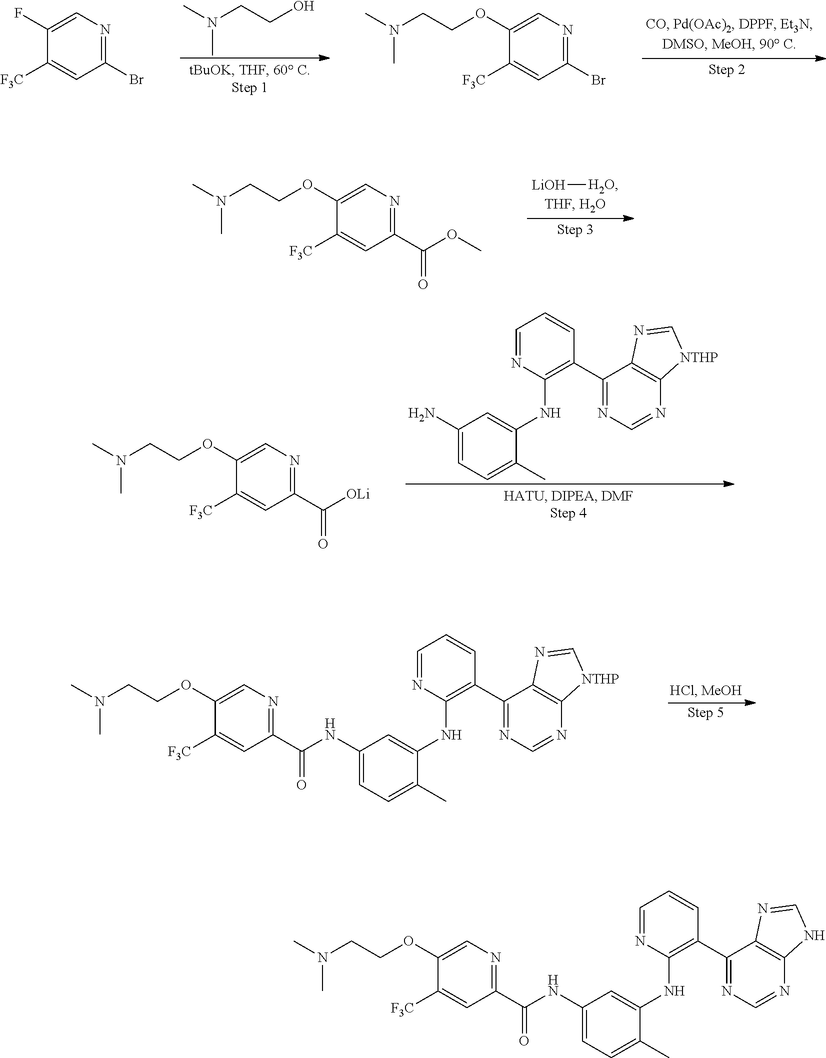

Step 1. The mixture of 2-bromo-5-fluoro-4-(trifluoromethyl)pyridine (244 mg, 1.0 mmol), 2-(dimethylamino)ethanol (98 mg, 1.1 mmol) and potassium tert-butoxide (1.1 mL, 1.1 mmol, 1 M in tetrahydrofuran) in tetrahydrofuran (5 mL) was stirred at 60° C. for 3 hours. The reaction mixture was concentrated. The residue was purified by flash chromatography (methanol in dichloromethane 1% to 8%) to afford 2-(6-bromo-4-(trifluoro methyl)pyridin-3-yloxy)-N,N-dimethylethanamine (300 mg, 0.96 mmol, 96%) as a white solid. MS (ESI) m/z 313.1 [M+H]⁺

Step 2. A mixture of 2-(6-bromo-4-(trifluoro methyl)pyridin-3-yloxy)-N,N-dimethylethanamine (300 mg, 0.96 mmol), 1, l′-bis(diphenylphosphino)ferrocene (213 mg, 0.384 mmol), palladium (II) acetate (43 mg, 0.192 mmol) and triethylamine (388 mg, 3.84 mmol) in dimethyl sulfoxide (12 mL) and methanol (8 mL) was stirred at 90° C. for 16 hours under carbon monoxide. The reaction mixture was cooled to room temperature, quenched with water (80 mL) and extracted with ethyl acetate (50 mL×6). The combined organic layers were dried over anhydrous sodium sulfate, filtered and concentrated under reduced pressure. The residue was purified by flash chromatography (methanol in dichloromethane 1% to 8%) to afford methyl 5-(2-(dimethylamino)ethoxy)-4-(trifluoromethyl)picolinate (214 mg, 0.73 mmol, 76%) as a solid. MS (ESI) m/z 292.8 [M+H]⁺

Step 3. To a solution of methyl 5-(2-(dimethylamino)ethoxy)-4-(trifluoromethyl)picolinate (74 mg, 0.25 mmol) in tetrahydrofuran (0.5 mL) were added lithium hydroxide hydrate (21 mg, 0.5 mmol) and water (0.5 mL) at 25° C. The reaction mixture was stirred at 25° C. for 2 hours and concentrated under reduced pressure to afford lithium 5-(2-(dimethylamino)ethoxy)-4-(trifluoromethyl)picolinate (95 mg) as a white solid, which was used without further purification. MS (ESI) m/z 279.3 [M+H]⁺

Step 4. To a solution 5-(2-(dimethylamino)ethoxy)-4-(trifluoromethyl)picolinate (95 mg, 0.33 mmol), 6-methyl-N1-(3-(9-(tetrahydro-2H-pyran-2-yl)-9H-purin-6-yl)pyridin-2-yl)benzene-1,3-diamine (76 mg, 0.19 mmol) and 1-[bis(dimethylamino)methylene]-1H-1,2,3-triazolo[4,5-b]pyridinium 3-oxid hexafluorophosphate (110 mg, 0.29 mmol) in N,N-dimethylformamide (3 mL) was added N,N-diisopropylethylamine (98 mg, 0.76 mmol) at 25° C. The reaction mixture was stirred at 25° C. for 1 hour, diluted with ethyl acetate (50 mL), washed with water (50 mL) and brine (50 mL×3), dried over sodium sulfate, filtered and concentrated. The residue was purified by flash chromatography (methanol/dichloromethane=8/100) to afford 5-(2-(dimethylamino)ethoxy)-N-(4-methyl-3-(3-(9-(tetrahydro-2H-pyran-2-yl)-9H-purin-6-yl)pyridin-2-ylamino)phenyl)-4-(trifluoromethyl)picolinamide (105 mg, 0.16 mmol, 84%) as a yellow solid. MS (ESI) m/z 661.7 [M+H]⁺

Step 5. To a solution of 5-(2-(dimethylamino)ethoxy)-N-(4-methyl-3-(3-(9-(tetrahydro-2H-pyran-2-yl)-9H-purin-6-yl)pyridin-2-ylamino)phenyl)-4-(trifluoromethyl)picolinamide (105 mg, 0.16 mmol) in methanol (10 mL) was added hydrochloric acid (3 M in methanol, 4 mL) at 25° C. The reaction mixture was stirred at 25° C. for 1 hour concentrated under reduced pressure. The residue was dissolved in methanol (10 mL), the pH was adjusted to 8 with ammonia (7 N in methanol). The mixture was concentrated under reduced pressure. The solid was collected by filtration and dried to afford N-(3-(3-(9H-purin-6-yl)pyridin-2-ylamino)-4-methylphenyl)-5-(2-(dimethylamino)ethoxy)-4-(trifluoromethyl)picolinamide (52.5 mg, 0.091 mmol, 57%) as a yellow solid. ¹H NMR (400 MHZ, DMSO-d₆) δ 13.45 (bs, 1H), 12.31 (s, 1H), 10.42 (s, 1H), 9.79 (s, 1H), 9.09 (s, 1H), 8.87-8.69 (m, 3H), 8.37 (d, J=3.1 Hz, 1H), 8.24 (s, 1H), 7.52 (d, J=7.1 Hz, 1H), 7.25 (d, J=8.2 Hz, 1H), 7.04 (dd, J=7.7, 4.7 Hz, 1H), 4.56 (t, J=4.8 Hz, 2H), 2.91-2.81 (m, 2H), 2.42 (s, 3H), 2.35 (s, 6H). MS (ESI) m/z 577.7 [M+H]⁺

### Example 95. Synthesis of N-(3-((3-(9H-purin-6-yl)pyridin-2-yl)amino)-4-methylphenyl)-6-(2-hydroxyethoxy)-4-(trifluoromethyl)picolinamide (Compound IA-98)

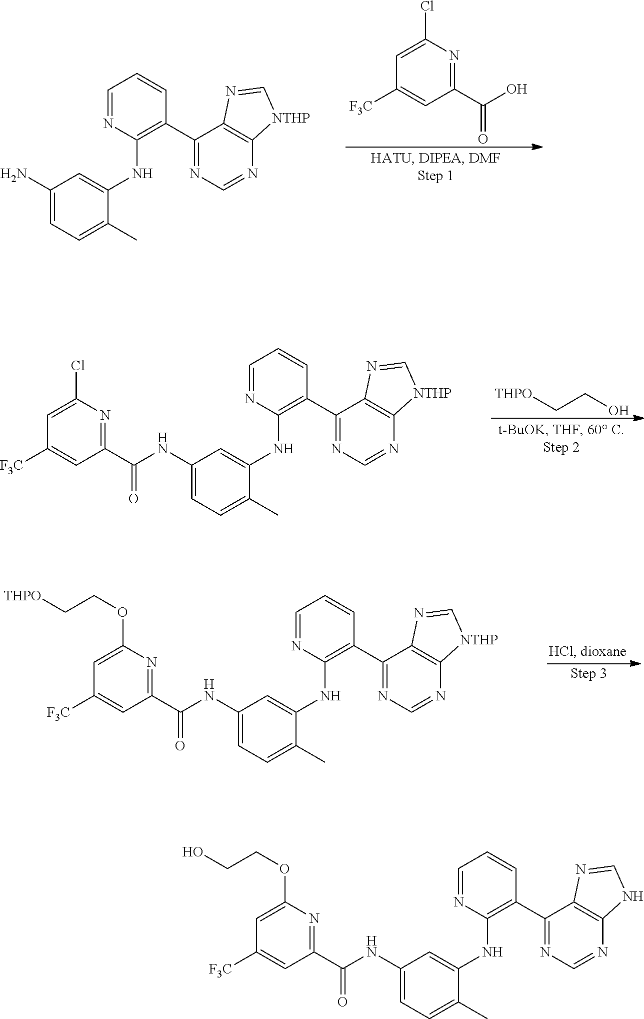

Step 1. To a mixture of 6-chloro-4-(trifluoromethyl)picolinic acid (50 mg, 0.22 mmol) in N,N-dimethylformamide (1 mL) was added 1-[bis(dimethylamino)methylene]-1H-1,2,3-triazolo[4,5-b]pyridinium 3-oxid hexafluorophosphate (101 mg, 0.26 mmol) and N,N-diisopropylethylamine (43 mg, 0.33 mmol) at room temperature. The mixture was stirred at room temperature for 5 minutes. 6-Methyl-N1-(3-(9-(tetrahydro-2H-pyran-2-yl)-9H-purin-6-yl)pyridin-2-yl) benzene-1,3-diamine (47 mg, 0.22 mmol) was added and the reaction mixture was stirred at room temperature for 3 hours. The mixture was diluted with water (5 mL), extracted with ethyl acetate (5 mL×3) and the combined organic phases were concentrated in vacuo. The residue was purified by flash chromatography on silica gel (dichloromethane/methanol=10/1) to afford 6-chloro-N-(4-methyl-3-((3-(9-(tetrahydro-2H-pyran-2-yl)-9H-purin-6-yl)pyridin-2-yl)amino)phenyl)-4-(trifluoro-methyl)picolinamide (30 mg, 0.05 mmol, 22%) as a yellow solid. MS (ESI) m/z 609.0 [M+H]⁺

Step 2. To a mixture of 2-((tetrahydro-2H-pyran-2-yl)oxy)ethan-1-ol (10 mg, 0.07 mmol) and potassium tert-butoxide (9 mg, 0.08 mmol) in tetrahydrofuran (1 mL) was added 6-chloro-N-(4-methyl-3-((3-(9-(tetrahydro-2H-pyran-2-yl)-9H-purin-6-yl)pyridin-2-yl)amino)phenyl)-4-(trifluoromethyl)picolinamide (40 mg, 0.07 mmol) at room temperature. The mixture was stirred at 100° C. for 16 hours. The reaction mixture was cooled to room temperature, diluted with water (5 mL), extracted with ethyl acetate (5 mL×3) and the combined organic phases were concentrated in vacuo. The residue was purified by flash chromatography on silica gel (dichloromethane/methanol=10/1) to afford N-(4-methyl-3-((3-(9-(tetrahydro-2H-pyran-2-yl)-9H-purin-6-yl)pyridin-2-yl)amino)phenyl)-6-(2-((tetrahydro-2H-pyran-2-yl)oxy)ethoxy)-4-(trifluoromethyl)picolinamide (35 mg, 0.05 mmol, 70%) as a yellow solid. MS (ESI) m/z 719.1 [M+H]⁺

Step 3. A solution of N-(4-methyl-3-((3-(9-(tetrahydro-2H-pyran-2-yl)-9H-purin-6-yl)pyridin-2-yl)amino)phenyl)-6-(2-((tetrahydro-2H-pyran-2-yl)oxy)ethoxy)-4-(trifluoromethyl)picolinamide (25 mg, 0.03 mmol) in hydrochloric acid (4 M in dioxane, 1 mL) was stirred at room temperature for 0.5 hours. The reaction mixture was concentrated under reduced pressure and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-6-(2-hydroxyethoxy)-4-(trifluoromethyl)picolinamide (2.0 mg, 0.0036 mmol, 12%) as a yellow solid. ¹H NMR (500 MHZ, DMSO-d₆) δ 13.87 (s, 1H), 12.36 (s, 1H), 10.28 (s, 1H), 9.83 (d, J=8.0 Hz, 1H), 9.10 (s, 1H), 8.76-8.73 (m, 1H), 8.36-8.35 (m, 1H), 7.89 (d, J=1.2 Hz, 1H), 7.49 (s, 1H), 7.45-7.43 (m, 1H), 7.28 (d, J=8.4 Hz, 1H), 7.06-7.03 (m, 1H), 4.94 (t, J=5.6 Hz, 1H), 4.64 (t, J=4.8 Hz, 2H), 3.82-3.79 (m, 2H), 2.45 (s, 3H). MS (ESI) m/z 551.0 [M+H]⁺

### Example 96. Synthesis of N-(3-(3-(9H-purin-6-yl)pyridin-2-ylamino)-4-methylphenyl)-2-(oxepan-3-yl)acetamide (Compound IA-99)

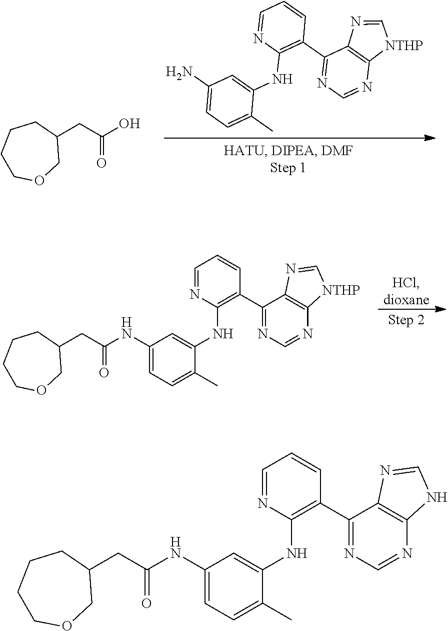

Step 1. To a solution of 2-(oxepan-3-yl)acetic acid (40 mg, 0.24 mmol), 6-methyl-N1-(3-(9-(tetrahydro-2H-pyran-2-yl)-9H-purin-6-yl)pyridin-2-yl)benzene-1,3-diamine (80 mg, 0.2 mmol) and 1-[bis(dimethylamino)methylene]-1H-1,2,3-triazolo[4,5-b]pyridinium 3-oxid hexafluorophosphate (140 mg, 0.36 mmol) in N,N-dimethylformamide (4 mL) was added diisopropylethylamine (0.4 mL) at room temperature. The mixture was stirred at room temperature for 1 hour. The mixture was diluted with water (10 mL) and extracted with ethyl acetate (10 mL×3). The combined organic layers were washed with brine (10 mL), dried over anhydrous sodium sulfate, filtered and concentrated. The residue was purified by flash chromatography (ethyl acetate) to afford N-(4-methyl-3-((3-(9-(tetrahydro-2H-pyran-2-yl)-9H-purin-6-yl)pyridin-2-yl)amino)phenyl)-2-(oxepan-3-yl)acetamide (100 mg, 93%) as a brown oil. MS (ESI) m/z 542.3 [M+H]⁺

Step 2. To a solution of N-(4-methyl-3-((3-(9-(tetrahydro-2H-pyran-2-yl)-9H-purin-6-yl)pyridin-2-yl)amino)phenyl)-2-(oxepan-3-yl)acetamide (100 mg, 0.18 mmol) in 1,4-dioxane (10 mL) was added hydrochloric acid solution (4 M in 1,4-dioxane, 5 mL) at room temperature. The mixture was stirred at room temperature for 1 hour and the solvent was removed under reduced pressure. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-2-(oxepan-3-yl)acetamide (35.9 mg, 0.079 mmol, 44%) as a yellow solid. ¹H NMR (500 MHZ, DMSO-d₆): δ12.30 (s, 1H), 9.82 (s, 1H), 9.79 (d, J=7.3 Hz, 1H), 9.03 (s, 1H), 8.67 (s, 1H), 8.43 (s, 1H), 8.33 (s, 1H), 7.38 (d, J=9.9 Hz, 1H), 7.14 (d, J=8.2 Hz, 1H), 7.02 (d, J=7.8 Hz, 1H), 3.69 (dd, J=12.1, 3.6 Hz, 1H),), 3.65-3.52 (m, 2H), 3.29-3.25 (m, 1H), 2.39 (s, 3H), 2.25-2.19 (m, 4H), 1.80-1.66 (m, 3H), 1.65-1.55 (m, 1H), 1.50-1.44 (m, 1H), 1.35-1.29 (m, 1H). MS (ESI) m/z 458.4 [M+H]⁺

### Example 97. Synthesis of N-(3-(3-(9H-purin-6-yl)pyridin-2-ylamino)-4-methylphenyl)-5-methoxy-4-(trifluoromethyl)picolinamide (Compound IA-100)

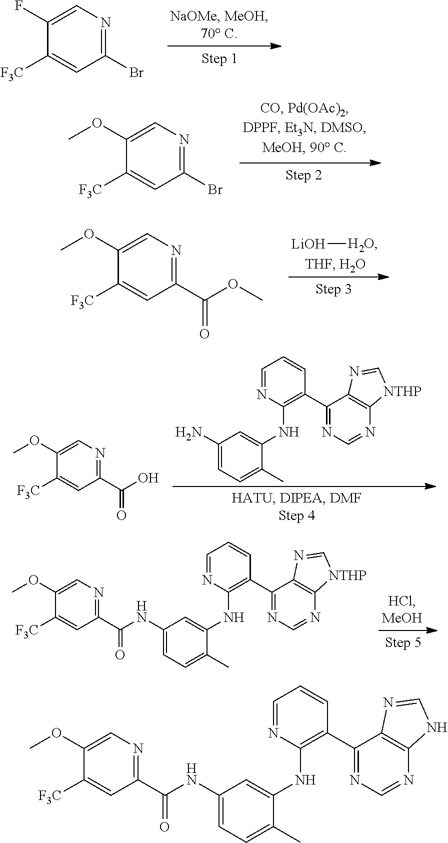

Step 1. The mixture of 2-bromo-5-fluoro-4-(trifluoromethyl)pyridine (200 mg, 0.82 mmol) and sodium methoxide (354 mg, 1.64 mmol, 25% in methanol) in methanol (4 mL) was stirred at 70° C. for 16 hours. The reaction mixture was concentrated. The residue was purified by flash chromatography (ethyl acetate/petroleum ether=3/10) to afford 2-bromo-5-methoxy-4-(trifluoromethyl)pyridine (173 mg, 0.67 mmol, 82%) as a white solid. MS (ESI) m/z 256.0 [M+H]⁺

Step 2. A mixture of 2-bromo-5-methoxy-4-(trifluoromethyl)pyridine (215 mg, 0.84 mmol), 1,1′-bis (diphenylphosphino)ferrocene (188 mg, 0.34 mmol), palladium (II) acetate (38 mg, 0.17 mmol) and triethylamine (340 mg, 3.36 mmol) in dimethyl sulfoxide (12 mL) and methanol (8 mL) was stirred at 90° C. for 16 hours under carbon monoxide. The reaction mixture was cooled to room temperature, quenched with water (80 mL) and extracted with ethyl acetate (50 mL×2). The combined organic layers were washed with brine (30 mL×3), dried over anhydrous sodium sulfate, filtered and concentrated under reduced pressure. The residue was purified by flash chromatography (ethyl acetate in petroleum ether from 10% to 30%) to afford methyl 5-methoxy-4-(trifluoromethyl)picolinate (155 mg, 0.66 mmol, 78%) as a white solid. MS (ESI) m/z 236.1 [M+H]⁺

Step 3. To a solution of methyl 5-methoxy-4-(trifluoromethyl)picolinate (155 mg, 0.66 mmol) in tetrahydrofuran (2 mL) were added lithium hydroxide hydrate (139 mg, 3.3 mmol) and water (2 mL) at 25° C. The reaction mixture was stirred at 25° C. for 16 hours and the solvent was evaporated under reduced pressure. The residue was diluted with water (10 mL) and the mixture was adjusted to pH 3 through the slow addition of 1 N aqueous hydrochloric acid. The resulting white precipitate was collected by filtration and washed with water (10 mL) to afford 5-methoxy-4-(trifluoromethyl)picolinic acid (120 mg, 0.54 mmol, 82%) as a white solid. MS (ESI) m/z 222.1 [M+H]⁺

Step 4. A solution of 6-methyl-N1-(3-(9-(tetrahydro-2H-pyran-2-yl)-9H-purin-6-yl)pyridin-2-yl)benzene-1,3-diamine (64 mg, 0.16 mmol), 5-methoxy-4-(trifluoromethyl)picolinic acid (55 mg, 0.16 mmol), 1-[bis(dimethylamino)methylene]-1H-1,2,3-triazolo[4,5-b]pyridinium 3-oxid hexafluorophosphate (91 mg, 0.24 mmol) and N,N-diisopropylethylamine (83 mg, 0.64 mmol) in N,N-dimethylformamide (3 mL) was stirred at 25° C. for 1 hour. The reaction mixture was diluted with ethyl acetate (50 mL), washed with water (50 mL) and brine (40 mL×3), dried over sodium sulfate, filtered and concentrated. The residue was purified by flash chromatography (ethyl acetate/petroleum ether=6/10) to afford 5-methoxy-N-(4-methyl-3-(3-(9-(tetrahydro-2H-pyran-2-yl)-9H-purin-6-yl)pyridin-2-ylamino)phenyl)-4 (trifluoromethyl)picolinamide (40 mg, 0.066 mmol, 41%) as a yellow solid. MS (ESI) m/z 605.0 [M+H]⁺

Step 5. To a solution of 5-methoxy-N-(4-methyl-3-(3-(9-(tetrahydro-2H-pyran-2-yl)-9H-purin-6-yl)pyridin-2-ylamino)phenyl)-4 (trifluoromethyl)picolinamide (40 mg, 0.066 mmol) in methanol (4 mL) was added methanolic hydrochloric acid (3 M, 2 mL) at 25° C. The mixture was stirred at 25° C. for 1 hour and concentrated. The residue was dissolved in methanol (30 mL), the pH was adjusted to 8 with ammonia (7 N in methanol). The mixture was concentrated under reduced pressure. The solid was collected by filtration and dried to afford N-(3-(3-(9H-purin-6-yl)pyridin-2-ylamino)-4-methylphenyl)-5-methoxy-4-(trifluoromethyl)-picolinamide (15 mg, 0.029 mmol, 44%) as a yellow solid. ¹H NMR (400 MHZ, DMSO-d₆) δ 13.87 (s, 1H), 12.34 (s, 1H), 10.43 (s, 1H), 9.83 (d, J=7.7 Hz, 1H), 9.09 (s, 1H), 8.79 (s, 2H), 8.73 (s, 1H), 8.37 (dd, J=4.6, 1.9 Hz, 1H), 8.24 (s, 1H), 7.52 (d, J=6.4 Hz, 1H), 7.25 (d, J=8.3 Hz, 1H), 7.04 (dd, J=7.8, 4.7 Hz, 1H), 4.17 (s, 3H), 2.44 (s, 3H). MS (ESI) m/z 520.8 [M+H]⁺

### Example 98. Synthesis of N-(3-(3-(9H-purin-6-yl)pyridin-2-ylamino)-4-methylphenyl)-3-methoxy-4-(trifluoromethyl)picolinamide (Compound IA-101)

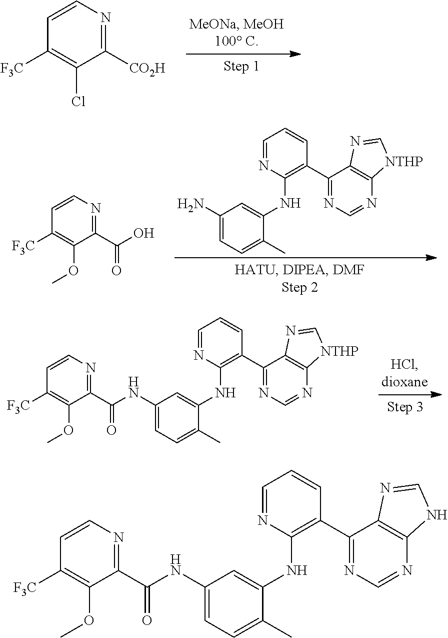

Step 1. A solution of 3-chloro-4-(trifluoromethyl)picolinic acid (1 g, 4.44 mmol) and sodium methoxide (480 mg, 8.88 mmol) in methanol (10 mL) was stirred at 100° C. for 48 hours. The mixture was poured into water (10 mL) and adjusted to pH 6 with dilute hydrochloric acid. The mixture was concentrated and purified by flash chromatography (petroleum ether/ethyl acetate=1/4) to afford 3-methoxy-4-(trifluoromethyl)picolinic acid (150 mg, 0.68 mmol, 15%) as a white solid. MS (ESI) m/z 222.2 [M+H]⁺

Step 2. A solution of 6-methyl-N1-(3-(9-(tetrahydro-2H-pyran-2-yl)-9H-purin-6-yl)pyridin-2-yl)benzene-1,3-diamine (273 mg, 0.18 mmol), 3-methoxy-4-(trifluoromethyl)picolinic acid (150 mg, 0.68 mmol), 1-[bis(dimethylamino)methylene]-1H-1,2,3-triazolo[4,5-b]pyridinium 3-oxid hexafluorophosphate (516 mg, 1.36 mmol) and N,N-diisopropylethylamine (263 mg, 2.04 mmol) in N,N-dimethylformamide (5 mL) was stirred at room temperature overnight. The reaction was poured into water (20 mL) and extracted with ethyl acetate (20 mL×3). The combined organic phases were concentrated and purified by flash chromatography (petroleum ether/ethyl acetate=1/3) to afford 3-methoxy-N-(4-methyl-3-((3-(9-(tetrahydro-2H-pyran-2-yl)-9H-purin-6-yl)pyridin-2-yl)amino)phenyl)-4-(trifluoromethyl) picolinamide (200 mg, 0.33 mmol, 48%) as a yellow solid. MS (ESI) m/z 605.3 [M+H]⁺

Step 3. A solution of- 3-methoxy-N-(4-methyl-3-((3-(9-(tetrahydro-2H-pyran-2-yl)-9H-purin-6-yl)pyridin-2-yl)amino)phenyl)-4-(trifluoromethyl)picolinamide (200 mg, 0.33 mmol) in hydrochloric acid (4M in dioxane, 2 mL) was stirred at room temperature overnight. The reaction mixture was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford crude product (15.0 mg, 0.03 mmol, 7.5%) as yellow solid. The crude product was further purified by chiral prep-HPLC (SFC-150 (Waters); Column: Diacel SC 20×250 mm, 10 μm; mobile phase: CO₂/MeOH (0.2% methanol ammonia)=40/60) to afford N-(3-((3-(9H-purin-6-yl)pyridin-2-yl)amino)-4-methylphenyl)-3-methoxy-4-(trifluoromethyl)picolinamide (2.0 mg, 0.004 mmol, 1%) as a yellow solid. ¹H NMR (400 MHz, CD₃OD) δ 12.53 (s, 1H), 10.62 (s, 1H), 9.84 (d, J=8.0 Hz, 1H), 8.98 (s, 1H), 8.68 (s, 1H), 8.63 (d, J=4.8 Hz, 1H), 8.57 (s, 1H), 8.31 (t, J=3.2 Hz, 1H), 7.88 (d, J=4.8 Hz, 1H), 7.51-7.48 (m, 1H), 7.24 (d, J=8.4 Hz, 1H), 7.03-6.66 (m, 2H), 5.32 (t, J=4.0 Hz, 1H), 3.96 (s, 3H), 4.45 (s, 3H). MS (ESI) m/z 521.1 [M+H]⁺

### Example 99. Synthesis of N-(3-(3-(9H-purin-6-yl)pyridin-2-ylamino)-4-methylphenyl)-2-(oxepan-4-yl)acetamide (Compound IA-102)

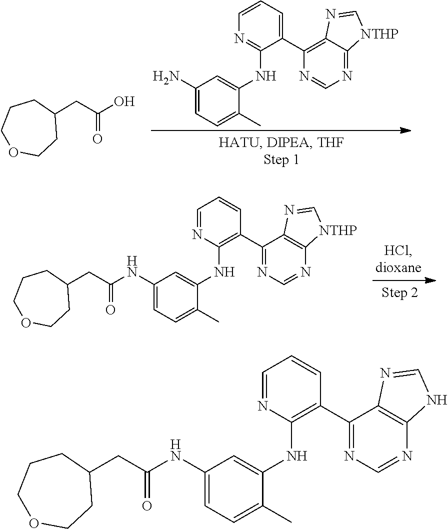

Step 1. To a mixture of 2-(oxepan-4-yl)acetic acid (70 mg, 0.44 mmol) in tetrahydrofuran (3 mL) were added N,N-diisopropylethylamine (114 mg, 0.88 mmol) and 1-[bis(dimethylamino)methylene]-1H-1,2,3-triazolo[4,5-b]pyridinium 3-oxid hexafluorophosphate (201 mg, 0.53 mmol) at 25° C. and the rection mixture was stirred for 0.5 h. Then 6-methyl-N1-(3-(9-(tetrahydro-2H-pyran-2-yl)-9H-purin-6-yl)pyridin-2-yl)benzene-1,3-diamine (176 mg, 0.44 mmol) was added and the mixture was stirred for 1 hour. The reaction mixture was concentrated and the residue was purified by flash chromatography (petroleum ether/ethyl acetate=1/1) to afford N-(4-methyl-3-(3-(9-(tetrahydro-2H-pyran-2-yl)-9H-purin-6-yl)pyridin-2-ylamino)phenyl)-2-(oxepan-4-yl)acetamide (150 mg, 0.28 mmol, 63%) as a yellow solid. MS (ESI) m/z 542.3 [M+H]⁺

Step 2. To a mixture of N-(4-methyl-3-(3-(9-(tetrahydro-2H-pyran-2-yl)-9H-purin-6-yl)pyridin-2-ylamino)phenyl)-2-(oxepan-4-yl)acetamide (150 mg, 0.28 mmol) in dioxane (3 mL) was added hydrochloric acid (4 M in dioxane, 5 mL) at 25° C. The reaction mixture was stirred for 2 hours and then concentrated. The residue was purified by prep-HPLC (Column: SunFire 4.6×50 mm C18, 3.5 μm; Mobile phase: acetonitrile/0.01% aqueous ammonium bicarbonate, 5%-95% over 1.5 min at 2 mL/min) to afford N-(3-(3-(9H-purin-6-yl)pyridin-2-ylamino)-4-methylphenyl)-2-(oxepan-4-yl)acetamide (62.6 mg, 0.14 mmol, 49%) as a yellow solid. ¹H NMR (400 MHZ, DMSO-d₆) δ 13.87 (s, 1H), 12.27 (s, 1H), 9.83-9.81 (m, 2H), 9.07 (s, 1H), 8.72 (s, 1H), 8.44 (d, J=4.0 Hz, 1H), 8.35 (dd, J=4.4 Hz, 1.6 Hz, 1H), 7.40 (dd, J=8.0 Hz, 2.0 Hz, 1H), 7.15 (d, J=8.4 Hz, 1H), 7.03 (dd, J=8.0 Hz, 4.8 Hz, 1H), 3.69-3.63 (m, 2H), 3.60-3.50 (m, 2H), 2.39 (s, 3H), 2.28 (d, J=7.2 Hz, 2H), 2.12-2.04 (m, 1H), 1.76-1.38 (m, 6H). MS (ESI) m/z 458.2 [M+H]⁺

### Example 100. Synthesis of (S)—N-(3-(3-(9H-purin-6-yl)pyridin-2-ylamino)-4-methylphenyl)-2-(3-(trifluoromethyl)piperidin-1-yl)acetamide (Compound IA-103)

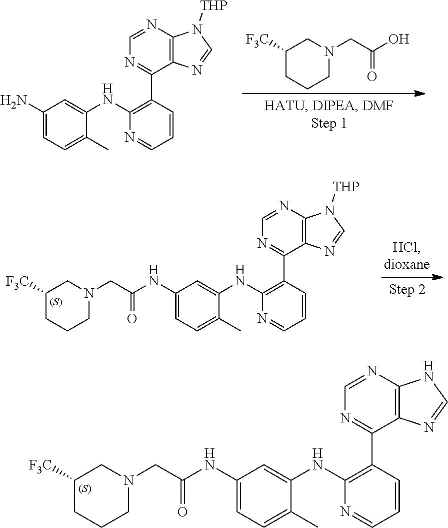

Step 1. To a solution of 6-methyl-N1-(3-(9-(tetrahydro-2H-pyran-2-yl)-9H-purin-6-yl)pyridin-2-yl)benzene-1,3-diamine (37.9 mg, 0.095 mmol) in N,N-dimethylformamide (3 mL) was added N,N-diisopropylethylamine (34.2 mg, 0.29 mmol), 1-[bis(dimethylamino)methylene]⁺ 1H-1,2,3-triazolo[4,5-b]pyridinium 3-oxid hexafluorophosphate (72 mg, 0.21 mmol) and (S)-2-(3-(trifluoromethyl)piperidin-1-yl)acetic acid (20 mg, 0.095 mmol). The mixture was stirred at room temperature for 1 hour. The reaction mixture was quenched with water (30 mL) and extracted with ethyl acetate (30 mL×3). The combined organic layers were washed with brine (30 mL), dried over anhydrous sodium sulfate, filtered and concentrated under reduced pressure to afford N-(4-methyl-3-((3-(9-(tetrahydro-2H-pyran-2-yl)-9H-purin-6-yl)pyridin-2-yl)amino)phenyl)-2-((S)-3-(trifluoromethyl)piperidin-1-yl)acetamide (42 mg, 0.10 mmol) as a yellow solid. MS (ESI) m/z 595.4 [M+H]⁺

Step 2. A mixture of N-(4-methyl-3-((3-(9-(tetrahydro-2H-pyran-2-yl)-9H-purin-6-yl)pyridin-2-yl)amino)phenyl)-2-((S)-3-(trifluoromethyl)piperidin-1-yl)acetamide (42 mg, 0.10 mmol) in hydrochloric acid (4 M in 1,4-dioxane, 5 mL) was stirred at room temperature for 2 hours. The reaction mixture was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to offer (S)—N-(3-((3-(9H-purin-6-yl)pyridin-2-yl)amino)-4-methylphenyl)-2-(3-(trifluoromethyl)piperidin-1-yl)acetamide (8.2 mg, 0.016 mmol) as a yellow solid. ¹H NMR (400 MHZ, DMSO-d₆) δ 12.38-12.16 (m, 1H), 9.93-9.69 (m, 1H), 9.64 (s, 1H), 9.07 (s, 1H), 8.72 (s, 1H), 8.48 (s, 1H), 8.38-8.27 (m, 1H), 7.44-7.30 (m, 1H), 7.19 (s, 1H), 7.11-6.94 (m, 1H), 3.19 (d, J=11.5 Hz, 2H), 3.09-2.99 (m, 1H), 2.89-2.77 (m, 1H), 2.68-2.61 (m, 1H), 2.40 (s, 3H), 2.29-2.12 (m, 2H), 1.94-1.80 (m, 1H), 1.78-1.66 (m, 1H), 1.64-1.52 (m, 1H), 1.23 (s, 1H). MS (ESI) m/z 511.2 [M+H]⁺

### Example 101. Synthesis of N-(3-(3-(9H-purin-6-yl)pyridin-2-ylamino)-4-methylphenyl)-5-(2-hydroxyethoxy)-4-(trifluoromethyl)picolinamide (Compound IA-104)

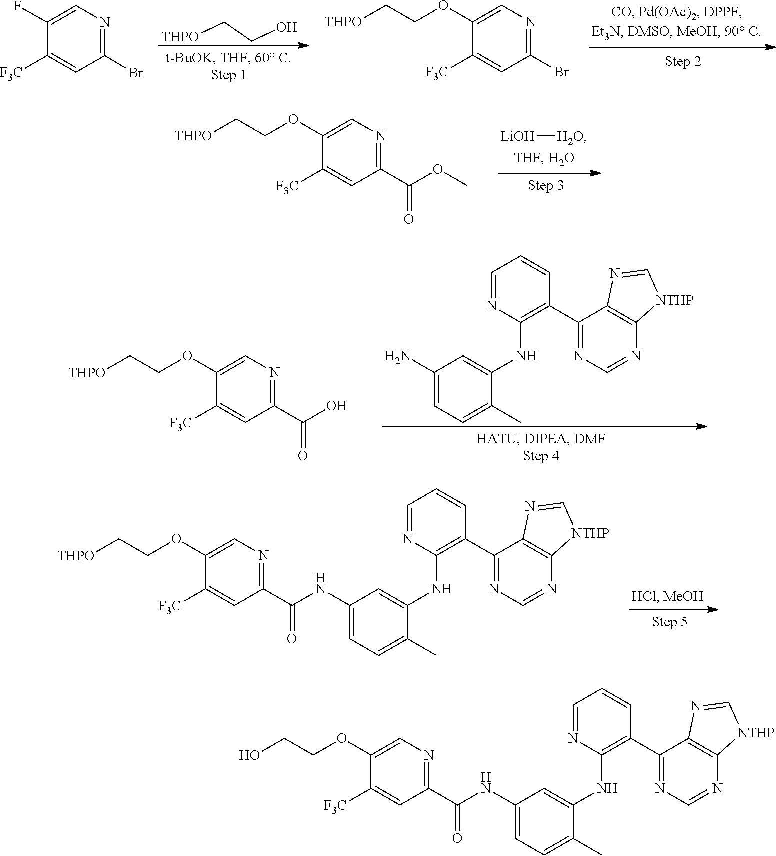

Step 1. A mixture of 2-bromo-5-fluoro-4-(trifluoromethyl)pyridine (300 mg, 1.2 mmol), 2-(tetrahydro-2H-pyran-2-yloxy)ethanol (193 mg, 1.32 mmol) and potassium tert-butoxide (1.32 mL, 1.32 mmol, 1 M in tetrahydrofuran) in tetrahydrofuran (10 mL) was stirred at 60° C. for 16 hours. The reaction mixture was concentrated and the residue was purified by flash chromatography (ethyl acetate/petroleum ether=30/100) to afford 2-bromo-5-(2-(tetrahydro-2H-pyran-2-yloxy)ethoxy)-4-(trifluoromethyl)pyridine (250 mg, 0.68 mmol, 57%) as a white solid. MS (ESI) m/z 369.9 [M+H]⁺

Step 2. A mixture of 2-bromo-5-(2-(tetrahydro-2H-pyran-2-yloxy)ethoxy)-4-(trifluoromethyl)-pyridine (200 mg, 0.54 mmol), 1,1′-bis(diphenylphosphino)ferrocene (120 mg, 0.216 mmol), palladium (II) acetate (24 mg, 0.108 mmol) and triethylamine (218 mg, 2.16 mmol) in dimethyl sulfoxide (12 mL) and methanol (8 mL) was stirred at 90° C. for 16 hours under carbon monoxide. The reaction mixture was cooled to room temperature, quenched with water (80 mL) and extracted with ethyl acetate (50 mL×2). The combined organic layers were washed with brine (30 mL×3), dried over anhydrous sodium sulfate, filtered and concentrated under reduced pressure. The residue was purified by flash chromatography (ethyl acetate in petroleum from 10% to 30%) to afford methyl 5-(2-(tetrahydro-2H-pyran-2-yloxy)ethoxy)-4-(trifluoromethyl)picolinate (164 mg, 0.47 mmol, 87%) as a white solid. MS (ESI) m/z 350.0 [M+H]⁺

Step 3. To a solution of methyl 5-(2-(tetrahydro-2H-pyran-2-yloxy)ethoxy)-4-(trifluoromethyl)-picolinate (205 mg, 0.59 mmol) in tetrahydrofuran (2 mL) were added lithium hydroxide hydrate (124 mg, 2.95 mmol) and water (2 mL) at 25° C. The reaction mixture was stirred at 25° C. for 16 hours. The solvent was evaporated under reduced pressure and the residue was diluted with water (10 mL). The mixture was adjusted to pH 3 by the slow addition of 1 N aqueous hydrochloric acid and extracted with ethyl acetate (60 mL×2). The combined organic layers were dried over anhydrous sodium sulfate, filtered, and the filtrate was concentrated under reduced pressure to afford 5-(2-(tetrahydro-2H-pyran-2-yloxy)ethoxy)-4-(trifluoromethyl)picolinic acid (145 mg, 0.43 mmol, 73%) as white solid. MS (ESI) m/z 336.0 [M+H]⁺

Step 4. A solution of 6-methyl-N1-(3-(9-(tetrahydro-2H-pyran-2-yl)-9H-purin-6-yl)pyridin-2-yl)benzene-1,3-diamine (100 mg, 0.25 mmol), 5-(2-(tetrahydro-2H-pyran-2-yloxy)ethoxy)-4-(trifluoromethyl)picolinic acid (85 mg, 0.25 mmol), 1-[bis(dimethylamino)methylene]-1H-1,2,3-triazolo[4,5-b]pyridinium 3-oxid hexafluorophosphate (144 mg, 0.38 mmol) and N,N-diisopropylethylamine (129 mg, 1.0 mmol) in N,N-dimethylformamide (5 mL) was stirred at 25° C. for 1 hour. The mixture was diluted with ethyl acetate (60 mL), washed with water (50 mL) and brine (40 mL×3), dried over sodium sulfate, filtered and concentrated. The residue was purified by flash chromatography (ethyl acetate/petroleum ether=58/100) to afford N-(4-methyl-3-(3-(9-(tetrahydro-2H-pyran-2-yl)-9H-purin-6-yl)pyridin-2-ylamino)phenyl)-5-(2-(tetrahydro-2H-pyran-2-yloxy)ethoxy)-4-(trifluoromethyl)picolinamide (130 mg, 0.18 mmol, 72%) as a yellow solid. MS (ESI) m/z 719.2 [M+H]⁺

Step 5. To a solution of N-(4-methyl-3-(3-(9-(tetrahydro-2H-pyran-2-yl)-9H-purin-6-yl)pyridin-2-ylamino)phenyl)-5-(2-(tetrahydro-2H-pyran-2-yloxy)ethoxy)-4-(trifluoromethyl)picolinamide (130 mg, 0.18 mmol) in methanol (10 mL) was added hydrochloric acid (3 M in methanol, 5 mL) at 25° C. The mixture was stirred for 1 hour and concentrated. The residue was dissolved in methanol (50 mL), the pH was adjusted to 8 with ammonia (7 N in methanol) and the mixture was concentrated under reduced pressure. The residue was triturated with methanol (50 mL), filtered and dried to afford N-(3-(3-(9H-purin-6-yl)pyridin-2-ylamino)-4-methylphenyl)-5-(2-hydroxyethoxy)-4-(trifluoromethyl)picolinamide (55.4 mg, 0.1 mmol, 55%) as a yellow solid. ¹H NMR (400 MHZ, DMSO-d₆) δ 13.87 (s, 1H), 12.34 (s, 1H), 10.42 (s, 1H), 9.82 (s, 1H), 9.09 (s, 1H), 8.82 (s, 1H), 8.78 (d, J=1.9 Hz, 1H), 8.73 (s, 1H), 8.37 (dd, J=4.7, 1.9 Hz, 1H), 8.23 (s, 1H), 7.53 (dd, J=8.2, 2.0 Hz, 1H), 7.25 (d, J=8.4 Hz, 1H), 7.04 (dd, J=7.9, 4.7 Hz, 1H), 5.01 (s, 1H), 4.47 (t, J=4.8 Hz, 2H), 3.80 (t, J=4.8 Hz, 2H), 2.44 (s, 3H). MS (ESI) m/z 551.0 [M+H]⁺

### Example 102. Synthesis of N-(3-((3-(9H-purin-6-yl)pyridin-2-yl)amino)-4-methylphenyl)-2-(4-(trifluoromethyl)tetrahydro-2H-pyran-2-yl)acetamide (Compound IA-105)

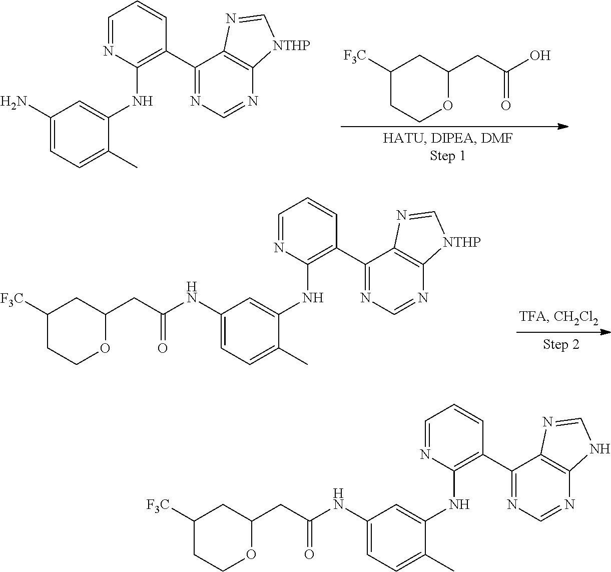

Step 1. 1-[Bis(dimethylamino)methylene]-1H-1,2,3-triazolo[4,5-b]pyridinium 3-oxid hexa-fluorophosphate (213 mg, 0.56 mmol) was added to a solution of 6-methyl-N1-(3-(9-(tetrahydro-2H-pyran-2-yl)-9H-purin-6-yl)pyridin-2-yl)benzene-1,3-diamine (150 mg, 0.37 mmol), 2-(4-(trifluoromethyl)tetrahydro-2H-pyran-2-yl)acetic acid (119 mg, 0.56 mmol) and N,N-diisopropylethylamine (193 mg, 1.49 mmol) in N,N-dimethylformamide (3 mL) at room temperature under nitrogen. The reaction mixture was stirred at room temperature for 1 hour and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(4-methyl-3-((3-(9-(tetrahydro-2H-pyran-2-yl)-9H-purin-6-yl)pyridin-2-yl)amino)phenyl)-2-(4-(trifluoromethyl)tetrahydro-2H-pyran-2-yl)acetamide (60 mg, 0.1 mmol, 27%) as a yellow solid. MS (ESI) m/z 512.3 [M+H]⁺

Step 2. Trifluoroacetic acid (2 mL) was added to a solution of N-(4-methyl-3-((3-(9-(tetrahydro-2H-pyran-2-yl)-9H-purin-6-yl)pyridin-2-yl)amino)phenyl)-2-(4-(trifluoromethyl)tetrahydro-2H-pyran-2-yl)acetamide (60 mg, 0.1 mmol) in dichloromethane (2 mL) at room temperature under nitrogen. The reaction mixture was stirred at room temperature for 2 hours and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-2-(4-(trifluoromethyl)tetrahydro-2H-pyran-2-yl)acetamide (18.5 mg, 0.036 mmol, 36%) as a yellow solid. ¹H NMR (400 MHZ, CD₃OD) δ=9.55 (d, J=7.6 Hz, 1H), 9.00 (s, 1H), 8.50 (s, 1H), 8.31 (d, J=2.0 Hz, 1H), 8.25 (m, 1H), 7.32 (m, 1H), 7.18 (d, J=8.0 Hz, 1H), 6.97 (m, 1H), 4.05 (m, 1H), 3.85 (m, 1H), 3.50 (m, 1H), 2.63-2.48 (m, 3H), 2.42 (s, 3H), 1.92 (m, 1H), 1.74 (m, 1H), 1.56 (m, 1H), 1.35 (m, 1H). MS (ESI) m/z 512.3 [M+H]⁺

### Example 103. Synthesis of N-(3-(3-(9H-purin-6-yl)pyridin-2-ylamino)-4-methylphenyl)-3-chloro-4-(trifluoromethyl)picolinamide (Compound IA-106)

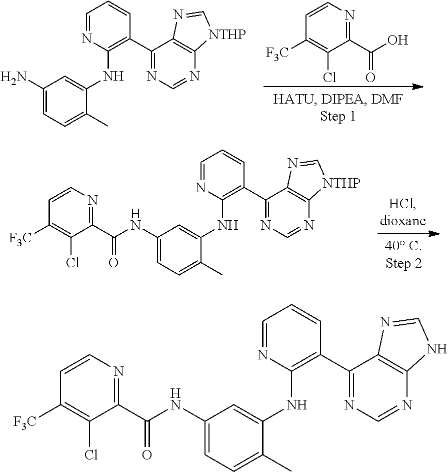

Step 1. A mixture of 6-methyl-N1-(3-(9-(tetrahydro-2H-pyran-2-yl)-9H-purin-6-yl)pyridin-2-yl)benzene-1,3-diamine (20.9 mg, 0.1 mmol), 3-chloro-4-(trifluoromethyl)picolinic acid (40.1 mg, 0.1 mmol), 1-[bis(dimethylamino)methylene]-1H-1,2,3-triazolo[4,5-b]pyridinium 3-oxid hexafluorophosphate (45.6 mg, 0.12 mmol) and N,N-diisopropylethylamine (38.7 mg, 0.3 mmol) in N,N-dimethylformamide (1 mL) was stirred at 25° C. for 2 hours. The reaction mixture was quenched with water (10 mL) and extracted with ethyl acetate (10 mL×3). The combined organic layers were washed with brine (10 mL), dried over sodium sulfate, filtered and concentrated. The residue was purified by flash chromatography (petroleum ether/ethyl acetate=1/1) to afford 3-chloro-N-(4-methyl-3-((3-(9-(tetrahydro-2H-pyran-2-yl)-9H-purin-6-yl)-pyridin-2-yl)amino)phenyl)-4-(trifluoromethyl)picolinamide (45 mg, 0.074 mmol, 74%) as a yellow solid. MS (ESI) m/z 609.2 [M+H]⁺

Step 2. A solution of 3-chloro-N-(4-methyl-3-((3-(9-(tetrahydro-2H-pyran-2-yl)-9H-purin-6-yl)pyridin-2-yl)amino)phenyl)-4-(trifluoromethyl)picolinamide (40 mg, 0.65 mmol) in hydrochloric acid (4 M in dioxane, 1 mL) was stirred at 40° C. overnight. The reaction mixture was concentrated, quenched with water (10 mL) and extracted with ethyl acetate (10 mL×3). The combined organic layers were washed with brine (10 mL), dried over sodium sulfate, filtered and concentrated. The residue was purified by purified by prep-HPLC (Column: BOSTON pHlex ODS 10 μm, 21.2×250 mm, 120 Å; Mobile phase: acetonitrile/0.1% ammonium bicarbonate) to afford N-(3-((3-(9H-purin-6-yl)pyridin-2-yl)amino)-4-methylphenyl)-3-chloro-4-(trifluoro-methyl)picolinamide (14.9 mg, 0.0283 mmol, 43%) as a yellow solid. ¹H NMR (400 MHZ, DMSO-d₆) δ 13.83 (s, 1H), 12.42 (s, 1H), 10.37 (s, 1H), 9.88 (s, 1H), 9.09 (s, 1H), 8.87-89 (d, 1H), 8.73 (s, 1H), 8.62-8.63 (s, 1H), 8.33-8.35 (d, 1H), 8.04-8.05 (s, 1H), 7.49-7.52 (d, 1H), 7.25-7.27 (d, 1H), 7.03-7.06 (d, 1H), 2.49 (s, 3H). MS (ESI) m/z 525.2 [M+H]⁺

### Example 104. Synthesis of (R)—N-(3-((3-(9H-purin-6-yl)pyridin-2-yl)amino)-4-methylphenyl)-2-(3-(trifluoromethyl)piperidin-1-yl)acetamide (Compound IA-107)

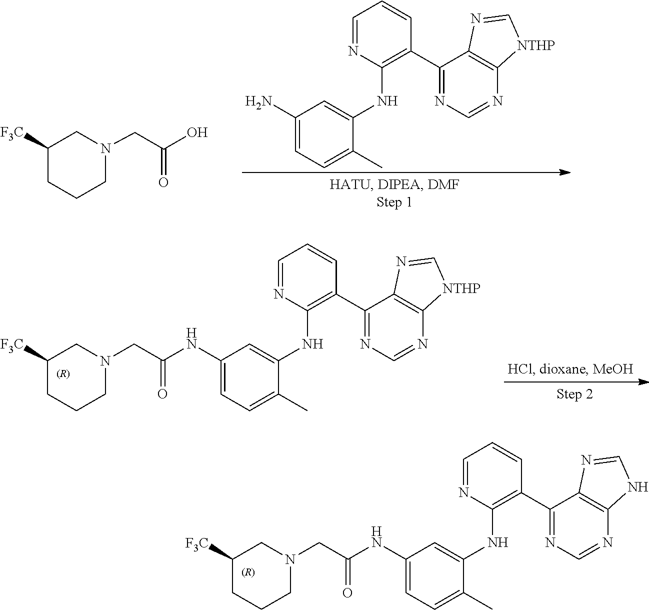

Step 1. To a solution of 6-methyl-N1-(3-(9-(tetrahydro-2H-pyran-2-yl)-9H-purin-6-yl)pyridin-2-yl)benzene-1,3-diamine (76 mg, 0.19 mmol) in N,N-dimethylformamide (4 mL) was added N,N-diisopropylethylamine (73 mg, 0.57 mmol), 1-[bis(dimethylamino)methylene]-1H-1,2,3-triazolo[4,5-b]pyridinium 3-oxid hexafluorophosphate (108 mg, 0.28 mmol) and (R)-2-(3-(trifluoromethyl)piperidin-1-yl)acetic acid (40 mg, 0.19 mmol). The reaction mixture was stirred at room temperature for 1 hour. The mixture was quenched with water (40 mL) and extracted with ethyl acetate (30 mL×3). The combined organic layers were washed with brine (30 mL), dried over anhydrous sodium sulfate, filtered and concentrated under reduced pressure. The residue was purified by flash chromatography (ethyl acetate) to afford N-(4-methyl-3-((3-(9-(tetrahydro-2H-pyran-2-yl)-9H-purin-6-yl)pyridin-2-yl)amino)phenyl)-2-((R)-3-(trifluoromethyl)piperidin-1-yl)acetamide (50 mg, 0.08 mmol, 45%) as a yellow solid. MS (ESI) m/z 595.0[M+H]⁺

Step 2. To a solution of N-(4-methyl-3-((3-(9-(tetrahydro-2H-pyran-2-yl)-9H-purin-6-yl)pyridin-2-yl)amino)phenyl)-2-((R)-3-(trifluoromethyl)piperidin-1-yl)acetamide (50 mg, 0.08 mmol) in methanol (4 mL) was added hydrochloric acid (4 M in dioxane, 4 mL) at 25° C. The reaction mixture was stirred for 1 hour and reaction mixture concentrated. The residue was dissolved in methanol (10 mL) and the pH was adjusted to 8 with ammonia (7 N in methanol). The mixture was concentrated under reduced pressure. The solid was collected by filtration and dried to afford (R)—N-(3-((3-(9H-purin-6-yl)pyridin-2-yl)amino)-4-methylphenyl)-2-(3-(trifluoro-methyl)piperidin-1-yl)acetamide (20.8 mg, 0.04 mmol, 70%) as a yellow solid. ¹H NMR (400 MHz, DMSO-d₆) δ 1.19-1.28 (m, 1H), 1.55-1.72 (m, 2H), 1.87 (d, J=11.6 Hz, 1H), 2.15-2.25 (m, 2H), 2.40 (s, 3H), 2.61-2.66 (m, 1H), 2.83 (d, J=10.0 Hz, 1H), 3.04 (d, J=8.4 Hz, 1H), 3.22 (q, J=15.2 Hz, 2H), 7.04 (q, J=4.8 Hz, 1H), 7.17 (d, J=4.8 Hz, 1H), 7.35 (d, J=8.0 Hz, 1H), 8.33 (d, J=2.8 Hz, 1H), 8.47 (s, 1H), 8.72 (s, 1H), 9.07 (s, 1H), 9.64 (s, 1H), 9.81 (d, J=8.0 Hz, 1H), 12.28 (s, 1H), 13.86 (s, 1H). MS (ESI) m/z 511.0 [M+H]⁺

### Example 105. Synthesis of N-(3-(3-(9H-purin-6-yl)pyridin-2-ylamino)-4-methylphenyl)-4-(trifluoromethyl)-2-oxabicyclo[2.1.1]hexane-1-carboxamide (Compound IA-108)

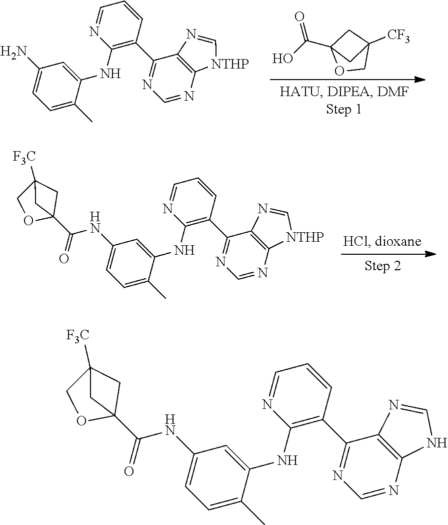

Step 1. To a solution of 4-(trifluoromethyl)-2-oxabicyclo[2.1.1]hexane-1-carboxylic acid (12 mg, 0.06 mmol) and 1-[bis(dimethylamino)methylene]-1H-1,2,3-triazolo[4,5-b]pyridinium 3-oxid hexafluorophosphate (34 mg, 0.09 mmol) in N,N-dimethylformamide (2 mL) was added 6-methyl-N1-(3-(9-(tetrahydro-2H-pyran-2-yl)-9H-purin-6-yl)pyridin-2-yl)benzene-1,3-diamine (20 mg, 0.05 mmol) and N,N-diisopropylethylamine (0.1 mL) at room temperature. The reaction mixture was stirred at room temperature for 1 hour, diluted with water (10 mL) and extracted with ethyl acetate (10 mL×3). The combined organic layers were washed with brine (10 mL), dried over anhydrous sodium sulfate, filtered and concentrated to afford N-(4-methyl-3-((3-(9-(tetrahydro-2H-pyran-2-yl)-9H-purin-6-yl)pyridin-2-yl)amino)phenyl)-4-(trifluoromethyl)-2-oxabicyclo[2.1.1]hexane-1-carboxamide (25 mg) as a brown oil, which was used without purification. MS (ESI) m/z 579.9 [M+H]⁺

Step 2. To a solution of N-(4-methyl-3-((3-(9-(tetrahydro-2H-pyran-2-yl)-9H-purin-6-yl)pyridin-2-yl)amino)phenyl)-4-(trifluoromethyl)-2-oxabicyclo[2.1.1]hexane-1-carboxamide (25 mg) in 1,4-dioxane (2 mL) was added hydrochloric acid (4 M in 1,4-dioxane, 1 mL) at room temperature. The reaction mixture was stirred at room temperature for 1 hour and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-4-(trifluoromethyl)-2-oxabicyclo[2.1.1]hexane-1-carboxamide (12.7 mg, 0.0256 mmol, 51%) as a yellow solid. ¹H NMR (400 MHZ, DMSO-d₆): δ13.87 (s, 1H), 12.30 (s, 1H), 9.79 (m, 2H), 9.08 (s, 1H), 8.72 (s, 1H), 8.60 (s, 1H), 8.36 (s, 1H), 7.36 (d, J=7.6 Hz, 1H), 7.19 (d, J=8.4 Hz, 1H), 7.06 (m, 1H), 4.03 (s, 2H), 2.48 (s, 2H), 2.41 (s, 3H), 2.12 (d, J=3.3 Hz, 2H). MS (ESI) m/z 495.9 [M+H]⁺

### Example 106. Synthesis of (S)—N-(3-((3-(9H-purin-6-yl)pyridin-2-yl)amino)-4-methylphenyl)-2-(3-(trifluoromethyl)pyrrolidin-1-yl)acetamide (Compound IA-109)

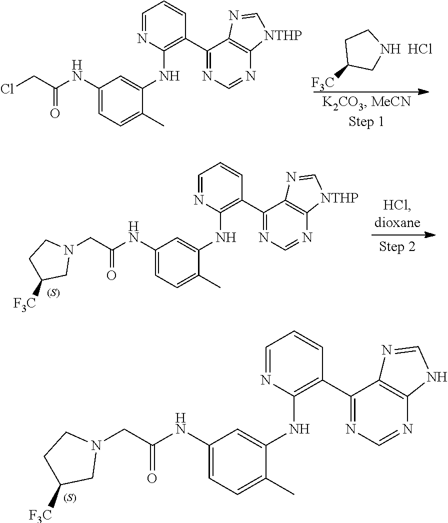

Step 1. A mixture of 2-chloro-N-(4-methyl-3-((3-(9-(tetrahydro-2H-pyran-2-yl)-9H-purin-6-yl)pyridin-2-yl)amino)phenyl)acetamide (98 mg, 0.20 mmol), (S)-3-(trifluoromethyl)pyrrolidine hydrochloride (30 mg, 0.17 mmol) and potassium carbonate (120 mg, 0.94 mmol) in acetonitrile (6 mL) was stirred at 70° C. for 24 hours. The reaction mixture was cooled to room temperature, quenched with water (30 mL) and extracted with ethyl acetate (10 mL×3). The combined organic layers were washed with brine (20 mL), dried over sodium sulfate, filtered and concentrated. The residue was purified by flash chromatography (methanol in dichloromethane, 0-10%), to afford N-(4-methyl-3-((3-(9-(tetrahydro-2H-pyran-2-yl)-9H-purin-6-yl)pyridin-2-yl)amino)phenyl)-2-((S)-3-(trifluoromethyl)pyrrolidin-1-yl)acetamide (50 mg, 0.086 mmol, 50%) as a yellow solid. MS (ESI) m/z 581.2 [M+H]⁺

Step 2. A solution of N-(4-methyl-3-((3-(9-(tetrahydro-2H-pyran-2-yl)-9H-purin-6-yl)pyridin-2-yl)amino)phenyl)-2-((S)-3-(trifluoromethyl)pyrrolidin-1-yl)acetamide (44 mg, 0.076 mmol) in hydrochloric acid (4 M in dioxane, 6 mL) was stirred at 20° C. for 1 hour. The reaction mixture was concentrated and the residue was treated with ammonia (7 N in methanol, 5 mL) and concentrated. The resulting solid was collected by filtration, washed with ethanol (20 mL) and water (5 mL) and dried to afford (S)—N-(3-((3-(9H-purin-6-yl)pyridin-2-yl)amino)-4-methylphenyl)-2-(3-(trifluoromethyl)pyrrolidin-1-yl)acetamide (27.7 mg, 0.056 mmol, 74%) as a yellow solid. ¹H NMR (400 MHZ, DMSO-d₆) δ 13.87 (s, 1H), 12.31 (s, 1H), 10.06-9.53 (m, 2H), 9.08 (s, 1H), 8.73 (s, 1H), 8.41 (d, J=61.6 Hz, 2H), 7.47-6.91 (m, 3H), 3.35-3.23 (m, 2H), 3.14 (s, 1H), 2.94 (s, 1H), 2.74 (s, 3H), 2.41 (s, 3H), 2.08 (s, 1H), 1.83 (s, 1H). MS (ESI) m/z 497.1 [M+H]⁺

### Example 107. Synthesis of N-(3-((3-(9H-purin-6-yl)pyridin-2-yl)amino)-4-methylphenyl)-2-(azepan-1-yl)acetamide (Compound IA-110)

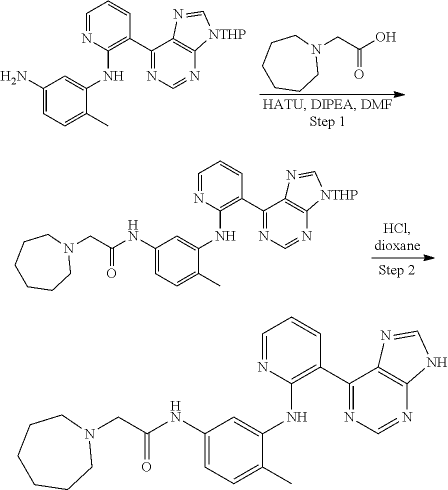

Step 1. To a mixture of 2-(azepan-1-yl) acetic acid (80 mg) in N,N-dimethylformamide (2 mL) was added 1-[bis(dimethylamino)methylene]-1H-1,2,3-triazolo[4,5-b]pyridinium 3-oxid hexafluorophosphate (232 mg, 0.61 mmol) and N,N-diisopropylethylamine (98 mg, 0.76 mmol) at room temperature. The reaction mixture was stirred for 5 minutes. 6-Methyl-N1-(3-(9-(tetrahydro-2H-pyran-2-yl)-9H-purin-6-yl)pyridin-2-yl)benzene-1,3-diamine (204 mg, 0.51 mmol) was added, and the reaction mixture was stirred at room temperature for 16 hours. The resulting mixture was diluted with water (20 mL) and extracted with ethyl acetate (20 mL×3). The combined organic phases were washed with brine (20 mL), dried over sodium sulfate, filtered, concentrated and purified by flash chromatography (dichloromethane/methanol=10/1) to afford 2-(azepan-1-yl)-N-(4-methyl-3-((3-(9-(tetrahydro-2H-pyran-2-yl)-9H-purin-6-yl)pyridin-2-yl)amino)phenyl)acetamide (110 mg, 0.20 mmol, 40%) as a yellow solid. MS (ESI) m/z 540.8 [M+H]⁺

Step 2. 2-(Azepan-1-yl)-N-(4-methyl-3-((3-(9-(tetrahydro-2H-pyran-2-yl)-9H-purin-6-yl)pyridin-2-yl)amino)phenyl)acetamide (50 mg, 0.09 mmol) was dissolved with hydrogen chloride (4 M in dioxane, 1 mL) and stirred at room temperature for 15 minutes. The solvent was removed under reduced pressure and the residue was treated with ammonia (8 M in methanol, 1 mL). The resulting mixture was filtered and the solid was dried under reduced pressure to afford N-(3-((3-(9H-purin-6-yl)pyridin-2-yl)amino)-4-methylphenyl)-2-(azepan-1-yl)acetamide (37.4 mg, 0.08 mmol, 91%) as a yellow solid. ¹H NMR (500 MHz, DMSO-d₆) δ 12.30 (s, 1H), 9.80-9.72 (m, 2H), 9.08 (s, 1H), 8.72 (s, 1H), 8.54 (s, 1H), 8.33 (d, J=2.8 Hz, 1H), 7.36 (d, J=8.8 Hz, 1H), 7.19 (d, J=8.4 Hz, 1H), 7.06-7.02 (m, 1H), 3.39 (s, 2H), 2.83 (s, 4H), 2.41 (s, 3H), 1.68-1.56 (m, 8H). MS (ESI) m/z 456.9 [M+H]⁺

### Example 108. Synthesis of (R)—N-(3-(3-(9H-purin-6-yl)pyridin-2-ylamino)-4-methylphenyl)-2-(3-(trifluoromethyl)pyrrolidin-1-yl)acetamide (Compound IA-111)

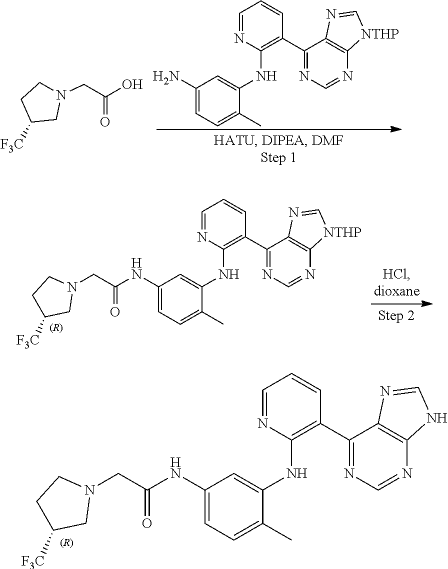

Step 1. A solution of (R)-2-(3-(trifluoromethyl)pyrrolidin-1-yl)acetic acid (20 mg, 0.10 mmol), 6-methyl-N1-(3-(9-(tetrahydro-2H-pyran-2-yl)-9H-purin-6-yl)pyridin-2-yl)benzene-1,3-diamine (40 mg, 0.10 mmol), 1-[bis(dimethylamino)methylene]-1H-1,2,3-triazolo[4,5-b]pyridinium 3-oxid hexafluorophosphate (42 mg, 0.11 mmol) and N,N-diisopropylethylamine (26 mg, 0.2 mmol) in N,N-dimethylformamide (2 mL) was stirred at room temperature for 1 hour. The reaction mixture was quenched with water (20 mL), extracted with ethyl acetate (80 mL), washed with brine (20 mL), dried over sodium sulfate, filtered and concentrated. The residue was purified by flash chromatography (ethyl acetate) to afford N-(4-methyl-3-((3-(9-(tetrahydro-2H-pyran-2-yl)-9H-purin-6-yl)pyridin-2-yl)amino)phenyl)-2-((R)-3-(trifluoromethyl)pyrrolidin-1-yl)acetamide (33 mg, 0.057 mmol, 57%) as a yellow solid. MS (ESI) m/z 581.0 [M+H]⁺

Step 2. A solution of N-(4-methyl-3-((3-(9-(tetrahydro-2H-pyran-2-yl)-9H-purin-6-yl)pyridin-2-yl)amino)phenyl)-2-((R)-3-(trifluoromethyl)pyrrolidin-1-yl)acetamide (33 mg, 0.057 mmol) in hydrochloric acid (4.0 M in dioxane, 2 mL) and methanol (1 mL) was stirred at room temperature for 2 hours. The mixture was concentrated in vacuo. The residue was dissolved in ammonia (7.0 M in methanol, 2 mL) and concentrated. The residue was triturated with methanol (3 mL) and dried to afford (R)—N-(3-((3-(9H-purin-6-yl)pyridin-2-yl)amino)-4-methylphenyl)-2-(3-(trifluoromethyl)pyrrolidin-1-yl)acetamide (13.3 mg, 0.026 mmol, 47.5%,) as a yellow solid. ¹H NMR (500 MHz, DMSO-d₆) δ 13.87 (s, 1H), 12.31 (s, 1H), 9.82 (dd, J=7.5 Hz, 2.0 Hz, 1H), 9.70 (s, 1H), 9.08 (s, 1H), 8.72 (s, 1H), 8.49 (s, 1H), 8.34 (dd, J=4.5 Hz, 1.5 Hz, 1H), 7.36 (d, J=6.5 Hz, 1H), 7.17 (d, J=8.5 Hz, 1H), 7.05-7.03 (m, 1H), 3.31 (s, 2H), 3.17-3.11 (m, 1H), 2.96-2.93 (m, 1H), 2.77-2.64 (m, 3H), 2.41 (s, 3H), 2.12-2.05 (m, 1H), 1.85-1.80 (m, 1H). MS (ESI) m/z 497.0 [M+H]⁺

### Example 109. Synthesis of N-(3-(3-(9H-purin-6-yl)pyridin-2-ylamino)-4-methylphenyl)-2-(3,3-difluoroazepan-1-yl)acetamide (Compound IA-112)

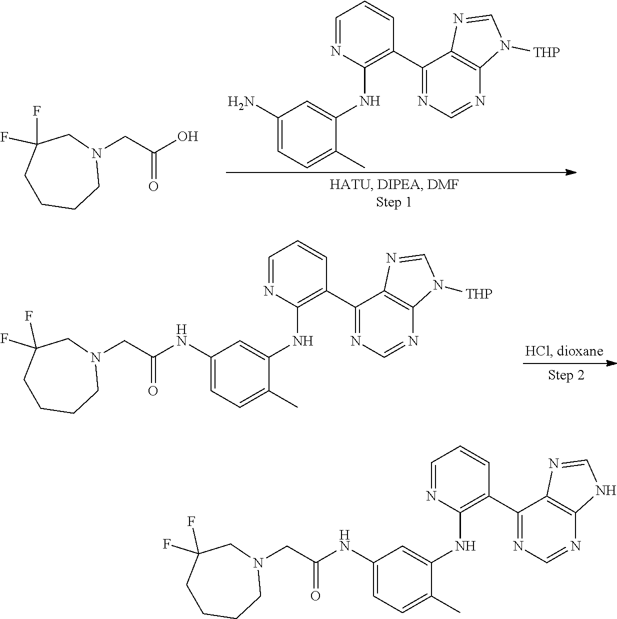

Step 1. A solution of 2-(3,3-difluoroazepan-1-yl)acetic acid (20 mg, 0.10 mmol), 6-methyl-N1-(3-(9-(tetrahydro-2H-pyran-2-yl)-9H-purin-6-yl)pyridin-2-yl)benzene-1,3-diamine (42 mg, 0.11 mmol), 1-[bis(dimethylamino)methylene]-1H-1,2,3-triazolo[4,5-b]pyridinium 3-oxid hexafluorophosphate (57 mg, 0.15 mmol) and N,N-diisopropylethylamine (32 mg, 0.25 mmol) in N,N-dimethylformamide (1 mL) was stirred at room temperature for 1 hour. The reaction solution was quenched with water (20 mL), extracted with ethyl acetate (80 mL), washed with brine (20 mL), dried over sodium sulfate, filtered and concentrated. The residue was purified by flash chromatography (ethyl acetate) to afford 2-(3,3-difluoroazepan-1-yl)-N-(4-methyl-3-((3-(9-(tetrahydro-2H-pyran-2-yl)-9H-purin-6-yl)pyridin-2-yl)amino)phenyl)acetamide (17 mg, 0.029 mmol, 30%) as a yellow solid. MS (ESI) m/z 577.2 [M+H]⁺

Step 2. A solution of 2-(3,3-difluoroazepan-1-yl)-N-(4-methyl-3-((3-(9-(tetrahydro-2H-pyran-2-yl)-9H-purin-6-yl)pyridin-2-yl)amino)phenyl)acetamide (17 mg, 0.029 mmol) in hydrochloric acid (4.0 M in dioxane, 2 mL) and methanol (1 mL) was stirred at room temperature for 2 hours. The reaction mixture was concentrated in vacuo and the residue was triturated with methanol (3 mL) to yield N-(3-((3-(9H-purin-6-yl) pyridin-2-yl)amino)-4-methylphenyl)-2-(3,3-difluoroazepan-1-yl)acetamide (3.9 mg, 0.008 mmol, 28%) as a yellow solid. ¹H NMR (500 MHZ, DMSO-d₆) δ 13.84 (s, 1H), 12.29 (s, 1H), 9.81 (dd, J=8.0 Hz, 1.5 Hz, 1H), 9.58 (s, 1H), 9.07 (s, 1H), 8.71 (s, 1H), 8.49 (s, 1H), 8.33 (dd, J=4.0 Hz, 1.5 Hz, 1H), 7.36 (d, J=8.5 Hz, 1H), 7.17 (d, J=8.5 Hz, 1H), 7.03 (dd, J=8.0 Hz, 0.5 Hz, 1H), 3.43 (s, 2H), 3.18 (t, J=7.0 Hz, 2H), 2.85 (t, J=6.5 Hz, 2H), 2.40 (s, 3H), 2.14-2.06 (m, 2H), 1.71-1.67 (m, 2H), 1.63-1.60 (m, 2H). MS (ESI) m/z 493.0 [M+H]⁺

### Example 110. Synthesis of N-(3-((3-(9H-purin-6-yl)pyridin-2-yl)amino)-4-methylphenyl)-2-cycloheptylacetamide (Compound IA-113)

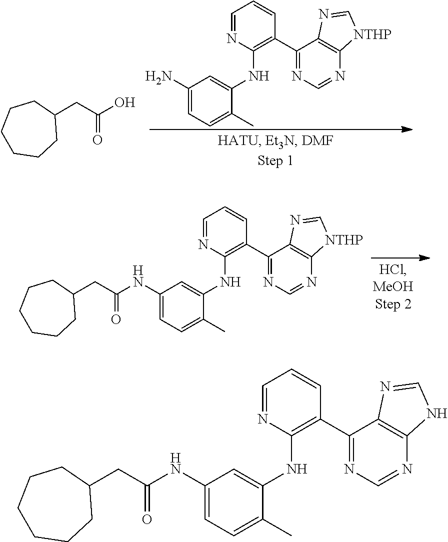

Step 1. A mixture of 2-cycloheptylacetic acid (50 mg, 0.32 mmol), 6-methyl-N1-(3-(9-(tetrahydro-2H-pyran-2-yl)-9H-purin-6-yl)pyridin-2-yl)benzene-1,3-diamine (128 mg, 0.32 mmol), 1-[bis(dimethylamino)methylene]-1H-1,2,3-triazolo[4,5-b]pyridinium 3-oxid hexa-fluorophosphate (365 mg, 0.96 mmol) and triethylamine (0.2 mL) in N,N-dimethylformamide (5 mL) was stirred at room temperature for 1 hour. The mixture was quenched with brine (30 mL) and extracted with ethyl acetate (30 mL×3). The combined organic phases were dried over sodium sulfate, filtered and concentrated under reduced pressure to afford 2-cycloheptyl-N-(4-methyl-3-((3-(9-(tetrahydro-2H-pyran-2-yl)-9H-purin-6-yl)pyridin-2-yl)amino)phenyl)acetamide (132 mg, 0.17 mmol, 76.5%) as a yellow oil. MS (ESI) m/z 539.8 [M+H]⁺

Step 2. A mixture of 2-cycloheptyl-N-(4-methyl-3-((3-(9-(tetrahydro-2H-pyran-2-yl)-9H-purin-6-yl)pyridin-2-yl)amino)phenyl)acetamide (132 mg, 0.17 mmol) in hydrochloric acid (3 M in methanol, 5 mL) was stirred at room temperature for 4 hours. The reaction mixture was concentrated, and the pH was adjusted to alkaline by adding ammonium (7 M in methanol). The residue was purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-2-cycloheptylacetamide as a yellow solid (7.1 mg, 0.016 mmol, 10.6%). ¹H NMR (400 MHZ, DMSO-d₆) δ 13.86 (s, 1H), 12.26 (s, 1H), 9.88-9.72 (m, 2H), 9.07 (s, 1H), 8.72 (s, 1H), 8.47-8.29 (m, 2H), 7.40 (dd, J=8.2, 1.8 Hz, 1H), 7.14 (d, J=8.3 Hz, 1H), 7.03 (dd, J=7.9, 4.7 Hz, 1H), 2.39 (s, 3H), 2.22 (d, J=7.3 Hz, 2H), 2.06-1.94 (m, 1H), 1.75-1.35 (m, 10H), 1.22 (ddd, J=13.3, 9.8, 4.8 Hz, 2H). MS (ESI) m/z 455.9 [M+H]⁺

### Example 111. Synthesis of N-(3-((3-(9H-purin-6-yl)pyridin-2-yl)amino)-4-methylphenyl)-5-methyl-4-(trifluoromethyl)picolinamide (Compound IA-114)

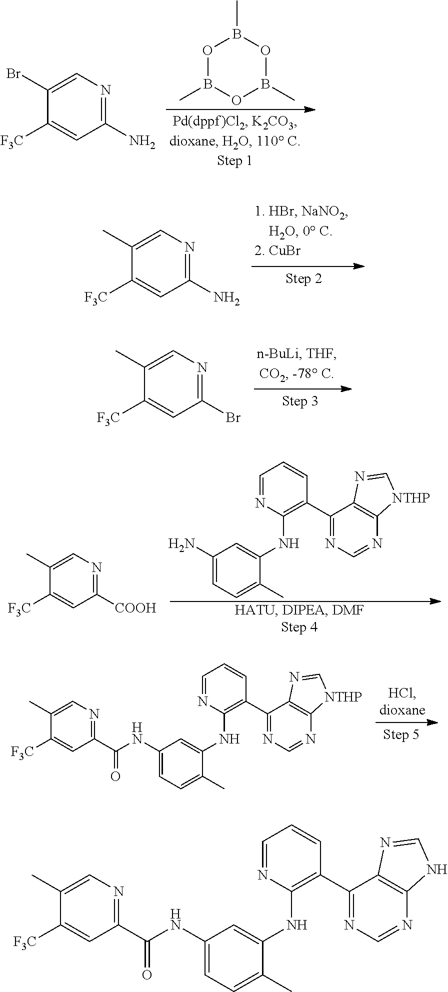

Step 1. To a mixture of 5-bromo-4-(trifluoromethyl)pyridin-2-amine (1.00 g, 4.15 mmol) in water (10 mL) and dioxane (40 mL) was added 2,4,6-trimethyl-1,3,5,2,4,6-trioxatriborinane (3.5 M in THF, 3.6 mL, 12.45 mmol), potassium carbonate (2.29 g, 16.60 mmol) and [1,1′-bis(diphenylphosphino)ferrocene]dichloro palladium(II) (304 mg, 0.41 mmol) at room temperature. The mixture was stirred at 110° C. for 16 hours. The reaction mixture was cooled to room temperature, filtered and the filtrate was concentrated. The residue was diluted with ethyl acetate (40 mL), washed with brine (20 mL), dried over sodium sulfate, filtered and concentrated. The residue was purified by flash chromatography (petroleum ether/ethyl acetate=2/1) to afford 5-methyl-4-(trifluoromethyl)pyridin-2-amine (260 mg, 1.48 mmol, 36%) as a yellow oil. MS (ESI) m/z 177.0 [M+H]⁺

Step 2. To a solution of 5-methyl-4-(trifluoromethyl)pyridin-2-amine (260 mg, 1.48 mmol) in hydrogen bromide (6 mL, 40% in water) was added sodium nitrite (204 mg, 2.95 mmol) at 0° C. The mixture was stirred at 0° C. for 1 hour and cuprous bromide (423 mg, 2.95 mmol) in hydrogen bromide (2 mL, 40% in water) was added. The reaction mixture was stirred at room temperature for 16 hours. The mixture was quenched by sodium hydroxide (1 N in water, 50 mL) and extracted with ethyl acetate (30 mL×3). The combined organic phases were concentrated and purified by flash chromatography (petroleum ether/ethyl acetate=10/1) to afford 2-bromo-5-methyl-4-(trifluoromethyl)pyridine (220 mg, 0.92 mmol, 62%) as a yellow oil. MS (ESI) m/z 239.9 [M+H]⁺

Step 3. To a solution of 2-bromo-5-methyl-4-(trifluoromethyl)pyridine (220 mg, 0.92 mmol) in tetrahydrofuran (2 mL) was added n-butyllithium (0.55 mL, 1.38 mmol, 2.5 M in hexanes) at −78° C. under argon. The mixture was stirred at −78° C. for 1 hour. Then dry ice was added, and the reaction mixture was stirred at room temperature for 16 hours. The reaction mixture was concentrated. The residue was diluted with ethyl acetate (10 mL), washed with water (5 mL), dried over sodium sulfate, filtered and concentrated under reduced pressure to afford 5-methyl-4-(trifluoromethyl)picolinic acid (110 mg, 0.54 mmol, 59%). MS (ESI) m/z 205.9 [M+H]⁺

Step 4. To a mixture of 5-methyl-4-(trifluoromethyl)picolinic acid (110 mg, 0.54 mmol) in N,N-dimethylformamide (5 mL) was added 1-[bis(dimethylamino)methylene]-1H-1,2,3-triazolo[4,5-b]pyridinium 3-oxid hexafluorophosphate (245 mg, 0.64 mmol) and N,N-diisopropylethylamine (104 mg, 0.80 mmol) at room temperature. The reaction mixture was stirred at room temperature for 5 minutes Then 6-methyl-N1-(3-(9-(tetrahydro-2H-pyran-2-yl)-9H-purin-6-yl)pyridin-2-yl)benzene-1,3-diamine (237 mg, 0.59 mmol) was added, and the mixture was stirred at room temperature for another 16 hours. The resulting mixture was diluted with water (20 mL) and extracted with ethyl acetate (20 mL×3). The combined organic phases were washed with brine (20 mL), dried over sodium sulfate, and filtered. The filtrate was concentrated and purified by flash chromatography (dichloromethane/methanol=10/1) to afford 5-methyl-N-(4-methyl-3-((3-(9-(tetrahydro-2H-pyran-2-yl)-9H-purin-6-yl)pyridin-2-yl)amino)phenyl)-4-(trifluoromethyl)picolinamide (60 mg, 0.10 mmol, 19%) as a yellow solid. MS (ESI) m/z 588.9 [M+H]⁺

Step 5. 5-methyl-N-(4-methyl-3-((3-(9-(tetrahydro-2H-pyran-2-yl)-9H-purin-6-yl)pyridin-2-yl)amino)phenyl-4-(trifluoromethyl)picolinamide (60 mg, 0.10 mmol) was dissolved in hydrogen chloride (4M in dioxane, 1 mL) and stirred at room temperature for 15 minutes. The reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-5-methyl-4-(trifluoromethyl)picolinamide (6.4 mg, 0.013 mmol, 13%) as a yellow solid. ¹H NMR (500 MHZ, DMSO-d₆) δ 13.82 (s, 1H), 12.35 (s, 1H), 10.59 (s, 1H), 9.79 (s, 1H), 9.07 (s, 1H), 8.89 (s, 1H), 8.82 (d, J=2.0 Hz, 1H), 8.70 (s, 1H), 8.37-8.36 (m, 1H), 8.27 (s, 1H), 7.55-7.52 (m, 1H), 7.25 (d, J=8.0 Hz, 3H), 7.06-7.02 (m, 1H), 2.58 (s, 3H), 2.33 (s, 3H). MS (ESI) m/z 505.0 [M+H]⁺

### Example 112. Synthesis of N-(3-((3-(9H-purin-6-yl)pyridin-2-yl)amino)-4-methylphenyl)-5-fluoro-4-(trifluoromethyl)picolinamide (Compound IA-115)

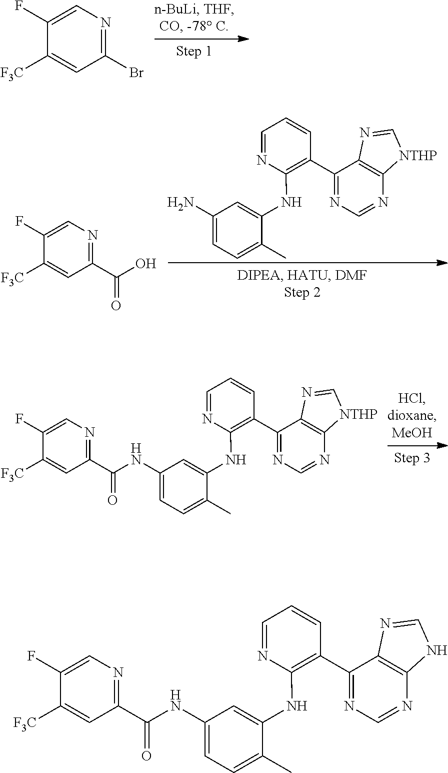

Step 1. To a solution of 2-bromo-5-fluoro-4-(trifluoromethyl)pyridine (100 mg, 0.41 mmol) in tetrahydrofuran (3 mL) was added n-butyllithium (2.5 M in hexane, 0.18 mL, 0.45 mmol) at −78° C. The reaction mixture was stirred at −78° C. for 30 minutes. Then fresh dry ice (10 g) was added, and the reaction mixture was stirred at −78° C. for another 1 hour. The reaction was quenched with water (30 mL) and washed with ether. The aqueous phase was acidified with dilute hydrochloric acid to pH 4 and extracted with ethyl acetate (20 mL×3). The combined organic phases were washed with water, dried over anhydrous sodium sulfate, filtered and concentrated to afford 5-fluoro-4-(trifluoromethyl)picolinic acid (40 mg, 0.19 mmol, 47%) as a white solid. MS (ESI) m/z 210.1 [M+H]⁺

Step 2. To a solution of 6-methyl-N1-(3-(9-(tetrahydro-2H-pyran-2-yl)-9H-purin-6-yl)pyridin-2-yl)benzene-1,3-diamine (77 mg, 0.19 mmol) in N,N-dimethylformamide (4 mL) were added N,N-diisopropylethylamine (74 mg, 0.57 mmol), 1-[bis(dimethylamino)methylene]-1H-1,2,3-triazolo[4,5-b]pyridinium 3-oxid hexafluorophosphate (109 mg, 0.28 mmol) and 5-fluoro-4-(trifluoromethyl)picolinic acid (40 mg, 0.19 mmol). The reaction mixture was stirred at room temperature for 1 hour, quenched with water (40 mL) and extracted with ethyl acetate (30 mL×3). The combined organic layers were washed with brine (30 mL), dried over anhydrous sodium sulfate, filtered and concentrated under reduced pressure. The residue was purified by flash chromatography (ethyl acetate) to afford 5-fluoro-N-(4-methyl-3-(3-(9-(tetrahydro-2H-pyran-2-yl)-9H-purin-6-yl)pyridin-2-ylamino)phenyl)-4-(trifluoromethyl)picolinamide (60 mg, 0.10 mmol, 53%) as a yellow solid. MS (ESI) m/z 592.9 [M+H]⁺

Step 3. To a solution of 5-fluoro-N-(4-methyl-3-(3-(9-(tetrahydro-2H-pyran-2-yl)-9H-purin-6-yl)pyridin-2-ylamino)phenyl)-4-(trifluoromethyl)picolinamide (60 mg, 0.10 mmol) in methanol (4 mL) was added hydrochloric acid (4 M in dioxane, 4 mL) at 25° C. The reaction mixture was stirred for 1 hour and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-5-fluoro-4-(trifluoromethyl)picolinamide (8.6 mg, 0.017 mmol, 17%) as a yellow solid. ¹H NMR (400 MHZ, DMSO-d₆) δ 2.45 (s, 3H), 7.04 (q, J=4.8 Hz, 1H), 7.26 (d, J=8.0 Hz, 1H), 7.49 (dd, J_{1=1.6} Hz, J_{2=8.2} Hz, 1H), 8.09 (t, J=4.8 Hz, 1H), 8.35 (dd, J₁=1.6 Hz, J₂=4.4 Hz, 1H), 8.69 (s, 1H), 8.72 (s, 1H), 8.80 (d, J=4.8 Hz, 1H), 9.09 (s, 1H), 9.82 (s, 1H), 10.67 (s, 1H), 12.35 (s, 1H), 13.86 (s, 1H). MS (ESI) m/z 508.8 [M+H]⁺

### Example 113. Synthesis of N-(3-((3-(9H-purin-6-yl)pyridin-2-yl)amino)-4-methylphenyl)-3-fluoro-4-(trifluoromethyl)picolinamide (Compound IA-116)

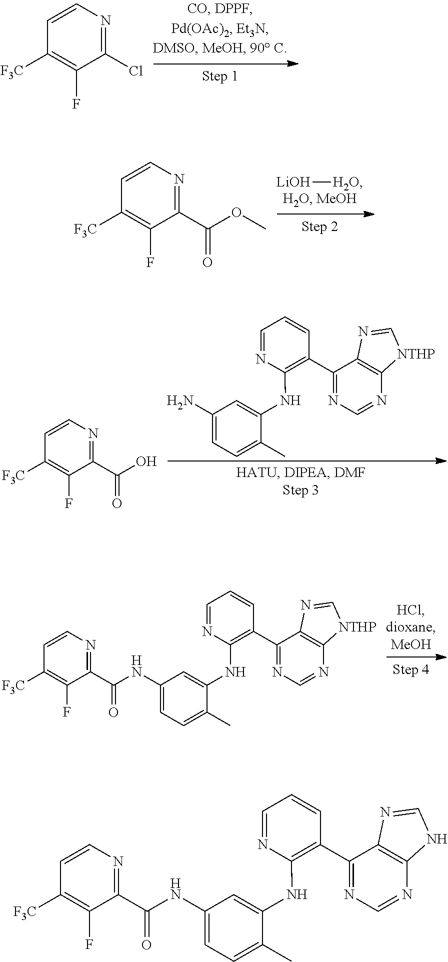

Step 1. A mixture of 2-chloro-3-fluoro-4-(trifluoromethyl)pyridine (200 mg, 1.01 mmol), 1, l′-bis(diphenylphosphino)ferrocene (111 mg, 0.20 mmol), palladium (II) acetate (23 mg, 0.10 mmol) and triethylamine (203 mg, 2.01 mmol) in dimethyl sulfoxide (6 mL) and methanol (2 mL) was stirred at 90° C. for 16 hours under carbon monoxide. The reaction mixture was quenched with water (50 mL) and extracted with ethyl acetate (50 mL×3). The combined organic layers were washed with brine (50 mL), dried over anhydrous sodium sulfate, filtered and concentrated under reduced pressure. The residue was purified by flash chromatography (petroleum ether/ethyl acetate=3/1) to afford methyl 3-fluoro-4-(trifluoromethyl) picolinate (90 mg, 0.4 mmol, 40%) as a yellow solid. MS (ESI) m/z 223.9 [M+H]⁺

Step 2. To a mixture of methyl 3-fluoro-4-(trifluoromethyl) picolinate (90 mg, 0.4 mmol) in methanol (4 mL) and water (2 mL) was added lithium hydroxide monohydrate (51 mg, 1.2 mmol) at room temperature and the reaction mixture was stirred for 1 hour. The organic phase was concentrated under reduced pressure and the remaining aqueous phase was acidified with 1 N hydrochloric acid to pH 6. The mixture was purified by prep-HPLC to afford 3-fluoro-4-(trifluoromethyl)picolinic acid (25 mg, 0.14 mmol, 30%) as a yellow solid. MS (ESI) m/z 210.0 [M+H]⁺

Step 3. To a solution of 6-methyl-N1-(3-(9-(tetrahydro-2H-pyran-2-yl)-9H-purin-6-yl)pyridin-2-yl)benzene-1,3-diamine (58 mg, 0.14 mmol) in N,N-dimethylformamide (6 mL) was added N,N-diisopropylethylamine (56 mg, 0.43 mmol), 1-[bis(dimethylamino)methylene]-1H-1,2,3-triazolo[4,5-b]pyridinium 3-oxid hexafluorophosphate (82 mg, 0.22 mmol) and 3-fluoro-4-(trifluoromethyl)picolinic acid (20 mg, 0.14 mmol) and the reaction mixture was stirred at room temperature for 1 hour. The mixture was quenched with water (30 mL) and extracted with ethyl acetate (30 mL×3). The combined organic layers were washed with brine (30 mL), dried over anhydrous sodium sulfate, filtered and concentrated under reduced pressure. The residue was purified by flash chromatography (ethyl acetate) to afford 3-fluoro-N-(4-methyl-3-((3-(9-(tetrahydro-2H-pyran-2-yl)-9H-purin-6-yl)pyridin-2-yl)amino)phenyl)-4-(trifluoromethyl)picolinamide (40 mg, 0.07 mmol, 71%) a as yellow solid. MS (ESI) m/z 592.8 [M+H]⁺

Step 4. To a solution of 3-fluoro-N-(4-methyl-3-((3-(9-(tetrahydro-2H-pyran-2-yl)-9H-purin-6-yl)pyridin-2-yl)amino)phenyl)-4-(trifluoromethyl)picolinamide (40 mg, 0.07 mmol) in methanol (4 mL) was added hydrochloric acid (4 M in dioxane, 4 mL) and the reaction mixture was stirred at 25° C. for 1 hour. The reaction mixture was concentrated, the residue was dissolved in methanol (10 mL) and the pH was adjusted to 8 with ammonia (7 N in methanol). The mixture concentrated under reduced pressure, and the solid was collected by filtration and dried to afford N-(3-((3-(9H-purin-6-yl)pyridin-2-yl)amino)-4-methylphenyl)-3-fluoro-4-(trifluoromethyl)picolinamide (13.9 mg, 0.027 mmol, 41%) as a yellow solid. ¹H NMR (400 MHZ, DMSO-d₆) δ 2.45 (s, 3H), 7.03-7.06 (m, 1H), 7.26 (d, J=8.4 Hz, 1H), 7.50 (d, J=7.6 Hz, 1H), 8.10 (t, J=4.4 Hz, 1H), 8.36 (d, J=3.2 Hz, 1H), 8.72 (d, J=6.8 Hz, 2H), 8.81 (d, J=4.8 Hz, 1H), 9.09 (s, 1H), 9.83 (d, J=7.2 Hz, 1H), 10.69 (s, H), 12.38 (s, 1H), 13.88 (s, 1H). MS (ESI) m/z 509.1 [M+H]⁺

### Example 114. Synthesis of N-(3-(4-fluoro-3-(9H-purin-6-yl)pyridin-2-ylamino)-4-methylphenyl)-4-(trifluoromethyl)picolinamide (Compound IA-117)

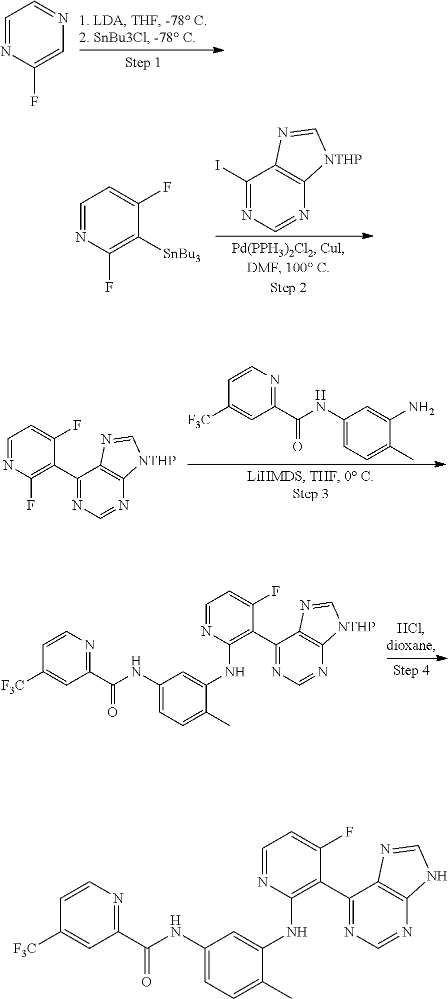

Step 1. Lithium diisopropylamide (2.0 M solution in tetrahydrofuran/n-heptane, 13 mL, 26 mmol) was added dropwise to a solution of 2,4-difluoropyridine (2 g, 17.4 mmol) in tetrahydrofuran (20 mL) at −78° C. The reaction mixture was stirred at −78° C. for 1 hour. Tributylchlorostannane (5.7 g, 17.4 mmol) in tetrahydrofuran (20 mL) was added, and the reaction mixture was stirred at −78° C. for 1 hour. The reaction mixture was quenched with saturated ammonium chloride solution (20 mL) and extracted with ethyl acetate (30 mL×3). The combined organic phases were washed with brine (30 mL), dried over sodium sulfate, filtered and concentrated. The residue was purified by flash chromatography (10% ethyl acetate in petroleum ether) to afford 2,4-difluoro-3-(tributylstannyl)pyridine (5.7 g, 14 mmol, 81%) as a yellow oil. MS (ESI) m/z 406.0 [M+H]⁺

Step 2. A solution of 2,4-difluoro-3-(tributylstannyl)pyridine (1.6 g, 4.0 mmol), 6-iodo-9-(tetrahydro-2H-pyran-2-yl)-9H-purine (1.3 g, 3.7 mmol), bis(triphenylphosphine)palladium(II) chloride (561 mg, 0.74 mmol) and cuprous iodide (151 mg, 0.74 mmol) in N,N-dimethylformamide (15 mL) was stirred at 100° C. for 16 hours under nitrogen. The reaction mixture was cooled to room temperature, poured into water (30 mL) and extracted with ethyl acetate (30 mL×3). The combined organic phases were washed with brine (30 mL), dried over sodium sulfate, filtered and concentrated. The residue was purified by flash chromatography (ethyl acetate/petroleum ether=1/5) to afford 6-(2,4-difluoropyridin-3-yl)-9-(tetrahydro-2H-pyran-2-yl)-9H-purine (1.2 g, 3.78 mmol, 94%) as a yellow oil. MS (ESI) m/z 318.0 [M+H]⁺

Step 3. Lithium hexamethyldisilazide (1.0 mol/L in tetrahydrofuran, 4.7 mL, 4.7 mmol) was added to a solution of 6-(2,4-difluoropyridin-3-yl)-9-(tetrahydro-2H-pyran-2-yl)-9H-purine (300 mg, 0.95 mmol) and N-(3-amino-4-methylphenyl)-4-(trifluoromethyl)picolinamide (280 mg, 0.95 mmol) in tetrahydrofuran (10 mL) at 0° C. and the reaction mixture was stirred at 0° C. for 1 hour. The reaction mixture was quenched with water (10 mL) and extracted with dichloromethane (30 mL×3). The combined organic layers were over anhydrous sodium sulfate, filtered and concentrated. The residue was purified by flash chromatography (petroleum ether/ethyl acetate=4/1) to afford N-(3-((4-fluoro-3-(9-(tetrahydro-2H-pyran-2-yl)-9H-purin-6-yl)pyridin-2-yl)amino)-4-methylphenyl)-4-(trifluoromethyl)picolinamide (120 mg, 0.2 mmol, 21%) as a brown solid. MS (ESI) m/z 593.4 [M+H]⁺

Step 4. A mixture of N-(3-((4-fluoro-3-(9-(tetrahydro-2H-pyran-2-yl)-9H-purin-6-yl)pyridin-2-yl)amino)-4-methylphenyl)-4-(trifluoromethyl)picolinamide (120 mg, 0.2 mmol) in hydrogen chloride (4 M in 1,4-dioxane, 5 mL) was stirred at room temperature overnight. The reaction mixture was adjusted to pH 8 with saturated aqueous sodium bicarbonate solution and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((4-fluoro-3-(9H-purin-6-yl)pyridin-2-yl)amino)-4-methylphenyl)-4-(trifluoromethyl)picolinamide (25.4 mg, 0.05 mmol, 25%) as a yellow solid. ¹H NMR (400 MHZ, DMSO-d₆) δ 13.50 (s, 1H), 10.84 (s, 1H), 9.03 (s, 1H), 9.02 (s, 1H), 8.91 (s, 1H), 8.66 (s, 1H), 8.33 (s, 1H), 8.09-8.08 (m, 1H), 7.95 (d, J=5.6 Hz, 1H), 7.87 (d, J=1.6 Hz, 1H), 7.69-7.66 (m, 1H), 7.27 (d, J=8.4 Hz, 1H), 6.7 (d, J=6.0 Hz, 1H), 2.19 (s, 3H). MS (ESI) m/z 509.1 [M+H]⁺

### Example 115. Synthesis of N-(3-((5-fluoro-3-(9H-purin-6-yl)pyridin-2-yl)amino)-4-methylphenyl)-4-(trifluoromethyl)picolinamide (Compound IA-118)

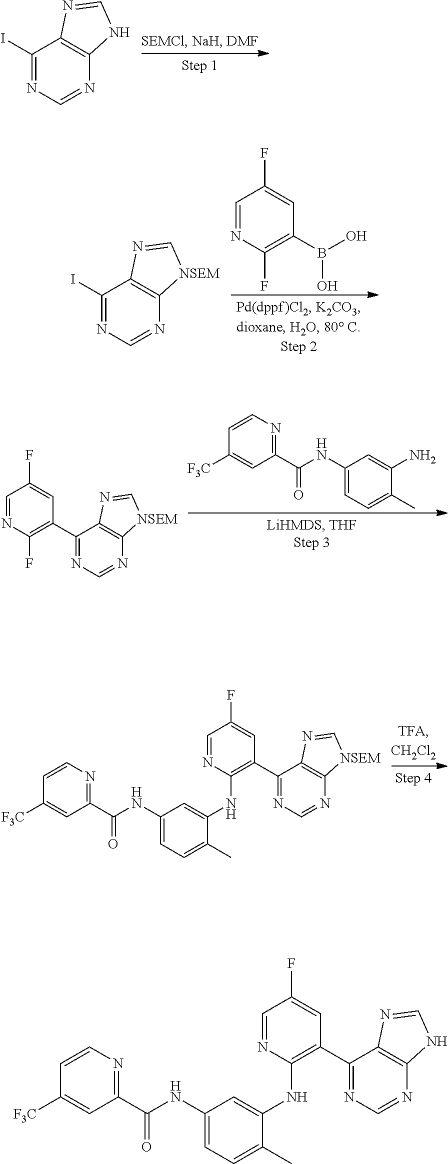

Step 1. To a solution of 6-iodo-9H-purine (10 g, 40.65 mmol) in N,N-dimethylformamide (100 mL) was added sodium hydride (60% dispersion in oil, 1.95 g, 48.78 mmol) under nitrogen at 0° C. The reaction mixture was stirred at room temperature for 10 minutes. Then 2-(trimethylsilyl)ethoxymethyl chloride (8.14 g, 0.84 mmol) was added, and the reaction mixture was stirred at room temperature for 2 hours. The resulting mixture was quenched with water (500 mL) and extracted with ethyl acetate (300 mL×3). The combined organic phases were washed with brine (100 mL), dried over sodium sulfate, filtered and concentrated. The residue was purified by flash chromatography (10% methanol in dichloromethane) to afford 6-iodo-9-((2-(trimethylsilyl)ethoxy)methyl)-9H-purine (6.00 g, 15.95 mmol, 33%) as a white solid. MS (ESI) m/z 377.1 [M+H]⁺

Step 2. To a solution of (2,5-difluoropyridin-3-yl)boronic acid (100 mg, 0.63 mmol) in dioxane (4 mL) and water (1 mL) was added 6-iodo-9-((2-(trimethylsilyl)ethoxy)methyl)-9H-purine (237 mg, 0.63 mmol), [1,1′-bis(diphenylphosphino)ferrocene] dichloropalladium (II) (44 mg, 0.06 mmol) and potassium carbonate (174 mg, 1.26 mmol) at room temperature and the reaction mixture was stirred at 80° C. for 16 hours. The reaction mixture was cooled to room temperature and filtered. The filtrate was poured into water (10 mL) and extracted with ethyl acetate (10 mL×3). The combined organic phases were concentrated and purified by flash chromatography (petroleum ether/ethyl acetate=5/1) to afford 6-(2,5-difluoropyridin-3-yl)-9-((2-(trimethylsilyl)ethoxy)methyl)-9H-purine (21 mg, 0.06 mmol, 9%) as a yellow oil. MS (ESI) m/z 364.1 [M+H]⁺

Step 3. To a solution of 6-(2,5-difluoropyridin-3-yl)-9-((2-(trimethylsilyl)ethoxy)methyl)-9H-purine (18 mg, 0.05 mmol) and N-(3-amino-4-methylphenyl)-4-(trifluoromethyl)picolinamide (15 mg, 0.05 mmol) in tetrahydrofuran (2 mL) was added lithium hexamethyldisilazide (0.25 mL, 0.25 mmol, 1 M in tetrahydrofuran) at 0° C. The mixture was stirred at room temperature for 16 hours. The solution was concentrated and purified by flash chromatography (petroleum ether/ethyl acetate=1/1) to afford N-(3-((5-fluoro-3-(9-((2-(trimethylsilyl)ethoxy)methyl)-9H-purin-6-yl)pyridin-2-yl)amino)-4-methylphenyl)-4-(trifluoromethyl)picolinamide (13 mg, 0.02 mmol, 41%) as a yellow solid. MS (ESI) m/z 638.5 [M+H]⁺

Step 4. A solution of N-(3-((5-fluoro-3-(9-((2-(trimethylsilyl)ethoxy)methyl)-9H-purin-6-yl)pyridin-2-yl)amino)-4-methylphenyl)-4-(trifluoromethyl)picolinamide (15 mg, 0.02 mmol) in hydrochloric acid (3 M in methanol, 1 mL) was stirred at room temperature for 1 hour. The solvent was removed under reduced pressure and the residue was treated with ammonia (1 mL, 8 M in methanol). The solid was collected by filtration and dried under reduced pressure to afford N-(3-((5-fluoro-3-(9H-purin-6-yl)pyridin-2-yl)amino)-4-methylphenyl)-4-(trifluoromethyl)picolinamide (4.3 mg, 0.008 mmol, 35%) as a yellow solid. ¹H NMR (500 MHZ, DMSO-d₆) δ 13.97 (s, 1H), 12.28 (s, 1H), 10.65 (s, 1H), 9.83-9.77 (m, 1H), 9.13 (s, 3H), 9.04 (d, J=4.4 Hz, 1H), 8.79-8.76 (m, 1H), 8.41 (d, J=2.8 Hz, 1H), 8.35 (s, 1H), 8.09 (d, J=5.6 Hz, 1H), 7.56 (dd, J=7.6 Hz, 1.6 Hz, 1H), 7.27 (d, J=8.0 Hz, 1H), 2.44 (s, 3H). MS (ESI) m/z 508.6 [M+H]⁺

### Example 116. N-(5-((3-(9H-purin-6-yl)pyridin-2-yl)amino)-2,4-dimethylphenyl)-4-(trifluoromethyl)picolinamide (Compound IA-119)

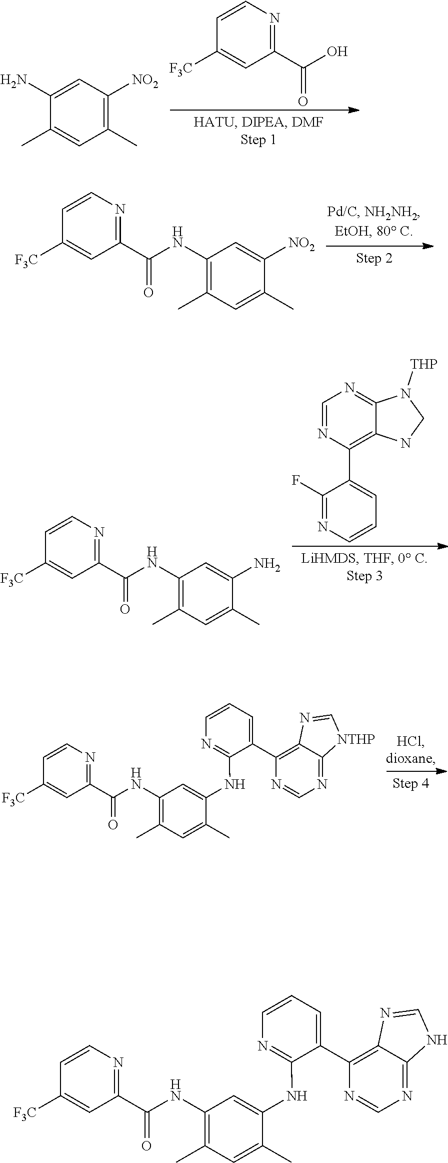

Step 1. A solution of 4-(trifluoromethyl)picolinic acid (500 mg, 2.6 mmol), 2,4-dimethyl-5-nitroaniline (415 mg, 2.5 mmol), 1-[bis(dimethylamino)methylene]-1H-1,2,3-triazolo[4,5-b]pyridinium 3-oxid hexafluorophosphate (1.5 g, 3.3 mmol) and N,N-diisopropylethylamine (1.7 mL) in N,N-dimethylformamide (15 mL) was stirred at room temperature for 1 hour. The mixture was diluted with ethyl acetate (50 mL) and washed with water (50 mL). The organic phase was washed with brine (20 mL), dried over sodium sulfate, filtered and concentrated. The residue was purified by flash chromatography (ethyl acetate/petroleum ether=1/1) to give N-(2,4-dimethyl-5-nitrophenyl)-4-(trifluoromethyl)picolinamide (750 mg, 85%) as a yellow solid. MS (ESI) m/z 339.7 [M+H]⁺

Step 2. A solution of N-(2,4-dimethyl-5-nitrophenyl)-4-(trifluoromethyl)picolinamide (740 mg, 2.2 mmol), hydrazine hydrate (3.6 ml) and palladium (10% on activated carbon, 360 mg) in ethanol (35 mL) was stirred at 80° C. for 1 hour. The reaction mixture was cooled, filtered and concentrated. The residue was diluted with ethyl acetate (30 mL) and washed with water (30 mL). The organic phase was washed with brine (40 mL), dried over sodium sulfate, filtered and concentrated to give N-(5-amino-2,4-dimethylphenyl)-4-(trifluoromethyl)picolinamide (660 mg, 2.14 mmol, 97%) as a yellow solid. MS (ESI) m/z 309.7 [M+H]⁺

Step 3. To a solution of N-(5-amino-2,4-dimethylphenyl)-4-(trifluoromethyl)picolinamide (155 mg, 0.5 mmol), 6-(2-fluoropyridin-3-yl)-9-(tetrahydro-2H-pyran-2-yl)-9H-purine (150 mg, 0.5 mmol) in tetrahydrofuran (10 mL) was added lithium hexamethyldisilazide (1.6 mL, 2.5 mmol, 1.6 M in tetrahydrofuran) at 0° C. slowly. The mixture was stirred at 0° C. for 1 hour. The reaction was quenched with saturated ammonium chloride solution (20 mL) at 0° C. and extracted with ethyl acetate (50 mL×3). The combined organic layers were washed with brine (50 mL), dried with anhydrous sodium sulfate, and filtered. The filtrate was concentrated under reduced pressure. The crude residue was purified flash chromatography (petroleum ether/ethyl acetate=3/7) to give N-(2,4-dimethyl-5-((3-(9-(tetrahydro-2H-pyran-2-yl)-9H-purin-6-yl)pyridin-2-yl)amino)phenyl)-4-(trifluoromethyl)picolinamide (210 mg, 0.357 mmol, 72%) as a yellow solid. MS (ESI) m/z 588.7 [M+H]⁺

Step 4. To a solution of N-(2,4-dimethyl-5-((3-(9-(tetrahydro-2H-pyran-2-yl)-9H-purin-6-yl)pyridin-2-yl)amino)phenyl)-4-(trifluoromethyl)picolinamide (45 mg, 0.076 mmol) in 1,4-dioxane (4 mL) was added hydrochloric acid (4 M in 1,4-dioxane, 1.5 mL). The mixture was stirred at room temperature for 1 hour and monitored by LCMS. After completion, the reaction mixture was concentrated and purified by prep-HPLC (Column: BOSTON pHlex ODS 10 μm, 21.2×250 mm, 120 Å; Mobile phase: acetonitrile/0.1% ammonium bicarbonate) to afford N-(5-((3-(9H-purin-6-yl)pyridin-2-yl)amino)-2,4-dimethylphenyl)-4-(trifluoromethyl)picolinamide (2.9 mg, 0.0058 mmol, 7.6%) as a yellow solid. ¹H NMR (500 MHz, DMSO-d₆): δ 13.85 (s, 1H), 12.31 (s, 1H), 10.36 (s, 1H), 9.82 (d, J=7.4 Hz, 1H), 9.09 (s, 1H), 9.05 (d, J=5.1 Hz, 1H), 8.71 (s, 1H), 8.67 (s, 1H), 8.35 (s, 1H), 8.31 (m, 1H), 8.10 (d, J=5.9 Hz, 1H), 7.18 (s, 1H), 7.00 (m, 1H), 2.44 (s, 3H), 2.24 (s, 3H). MS (ESI) m/z 504.7 [M+H]⁺

### Example 117. N-(3-(3-(9H-purin-6-yl)pyrazin-2-ylamino)-4-methylphenyl)-4-(trifluoromethyl)picolinamide (Compound IA-120)

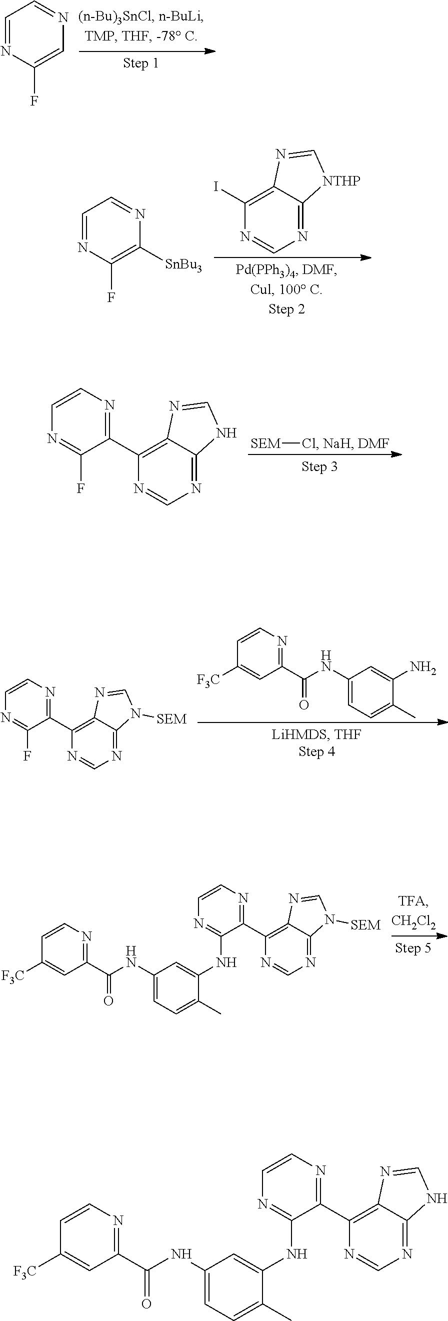

Step 1. To a solution of 2,2,6,6-tetramethylpiperidine (1.7 g, 12.24 mmol) in tetrahydrofuran (20 mL) at −78° C. was added n-butyllithium (2.5 M in hexane, 4.5 mL, 11.22 mmol) dropwise. The mixture was stirred at 0° C. for 20 minutes and then cooled to −78° C. A solution of 2-fluoropyrazine (1.0 g, 10.20 mmol) in tetrahydrofuran (5 mL) was added dropwise. After 5 minutes, tributyltin chloride (3.6 g, 11.22 mmol) was added and the reaction mixture was stirred at −78° C. for another 1 hour. The reaction was quenched with saturated ammonium chloride solution (30 mL) and extracted with ethyl acetate (30 mL×3). The combined organic phases were washed with brine (30 mL), dried over sodium sulfate, filtered, and concentrated. The residue was purified by flash chromatography (10% ethyl acetate in petroleum ether) to afford 2-fluoro-3-(tributylstannyl)pyrazine (2.5 g, 6.44 mmol, 63.2%) as a colorless oil. MS (ESI) m/z 387.1 [M+H]⁺

Step 2. A mixture of 2-fluoro-3-(tributylstannyl)pyrazine (500 mg, 1.29 mmol), 6-iodo-9-(tetrahydro-2H-pyran-2-yl)-9H-purine (426 mg, 1.29 mmol), tetrakis(triphenylphosphine)-palladium (150 mg, 0.13 mmol) and copper(I) iodide (25 mg, 0.13 mmol) in N,N-dimethylformamide (5 mL) was stirred at 100° C. for 16 hours. After cooling to room temperature, the mixture was filtered and purified by reversed-phase chromatography (15% acetonitrile in water, 0.1% ammonium bicarbonate) to afford 6-(3-fluoropyrazin-2-yl)-9H-purine (200 mg, 0.93 mmol, 71.8%) as a yellow solid. MS (ESI) m/z 217.2 [M+H]⁺

Step 3. To a solution of 6-(3-fluoropyrazin-2-yl)-9H-purine (120 mg, 0.56 mmol) in N,N-dimethylformamide (5 mL) was added sodium hydride (60% dispersion in oil, 29 mg, 0.73 mmol). After stirring at room temperature for 10 minutes, 2-(trimethylsilyl)ethoxymethyl chloride (140 mg, 0.84 mmol) was added. The mixture was stirred for another 3 hours. The resulting mixture was quenched with water (10 mL) and extracted with ethyl acetate (15 mL×3). The combined organic phases were washed with brine (10 mL), dried over sodium sulfate, filtered, and concentrated. The residue was purified by flash chromatography (5% methanol in dichloromethane) to afford 6-(3-fluoropyrazin-2-yl)-9-((2-(trimethylsilyl)ethoxy)methyl)-9H-purine (150 mg, 0.43 mmol, 77.4%) as a colorless oil. MS (ESI) m/z 347.1 [M+H]⁺

Step 4. To a solution of N-(3-amino-4-methylphenyl)-4-(trifluoromethyl)picolinamide (102 mg, 0.35 mmol) and 6-(3-fluoropyrazin-2-yl)-9-((2-(trimethylsilyl)ethoxy)methyl)-9H-purine (120 mg, 0.35 mmol) in tetrahydrofuran (10 mL) was added lithium hexamethyldisilazide (1.6 M in tetrahydrofuran, 1.1 mL, 1.75 mmol) at 0° C. slowly. The reaction was stirred at room temperature for 1 hour, quenched with saturated ammonium chloride solution (20 mL) at 0° C. and extracted with ethyl acetate (30 mL×3). The combined organic layers were washed with brine (20 mL), dried over anhydrous sodium sulfate, filtered, and concentrated. The residue was purified by flash chromatography (90% ethyl acetate in petroleum ether) to afford N-(4-methyl-3-((3-(9-((2-(trimethylsilyl)ethoxy)methyl)-9H-purin-6-yl)pyrazin-2-yl)amino)phenyl)-4-(trifluoromethyl)picolinamide (150 mg, 0.24 mmol, 69%) as a yellow solid. MS (ESI) m/z 622.1 [M+H]⁺

Step 5. A mixture of N-(4-methyl-3-((3-(9-((2-(trimethylsilyl)ethoxy)methyl)-9H-purin-6-yl)pyrazin-2-yl)amino)phenyl)-4-(trifluoromethyl)picolinamide (150 mg, 0.24 mmol) in dichloromethane (5 mL) and trifluoroacetic acid (1 mL) was stirred at room temperature for 3 hours. The reaction was concentrated under reduced pressure. The residue was treated with saturated sodium bicarbonate solution. The resulting mixture was extracted with ethyl acetate (15 mL×3). The combined organic layers were dried over sodium sulfate, filtered, concentrated and purified by flash chromatography (5% methanol in dichloromethane) and then triturated with methanol to afford N-(3-((3-(9H-purin-6-yl)pyrazin-2-yl)amino)-4-methylphenyl)-4-(trifluoromethyl)picolinamide (7.3 mg, 0.015 mmol, 6.2%) as a yellow solid. ¹H NMR (400 MHZ, DMSO-d₆) 13.38 (s, 1H), 12.24 (s, 1H), 10.73 (s, 1H), 9.21 (s, 1H), 9.05 (d, J=5.0 Hz, 1H), 8.85 (d, J=2.0 Hz, 1H), 8.81 (s, 1H), 8.44 (d, J=2.3 Hz, 1H), 8.36 (s, 1H), 8.33 (d, J=2.3 Hz, 1H), 8.10 (d, J=5.0 Hz, 1H), 7.60 (dd, J=8.2, 2.1 Hz, 1H), 7.32 (d, J=8.4 Hz, 1H), 2.49 (s, 3H). MS (ESI) m/z 491.7 [M+H]⁺

### Example 118. N-(3-((4-(9H-purin-6-yl)pyridazin-3-yl)amino)-4-methylphenyl)-4-(trifluoromethyl)picolinamide (Compound IA-121)

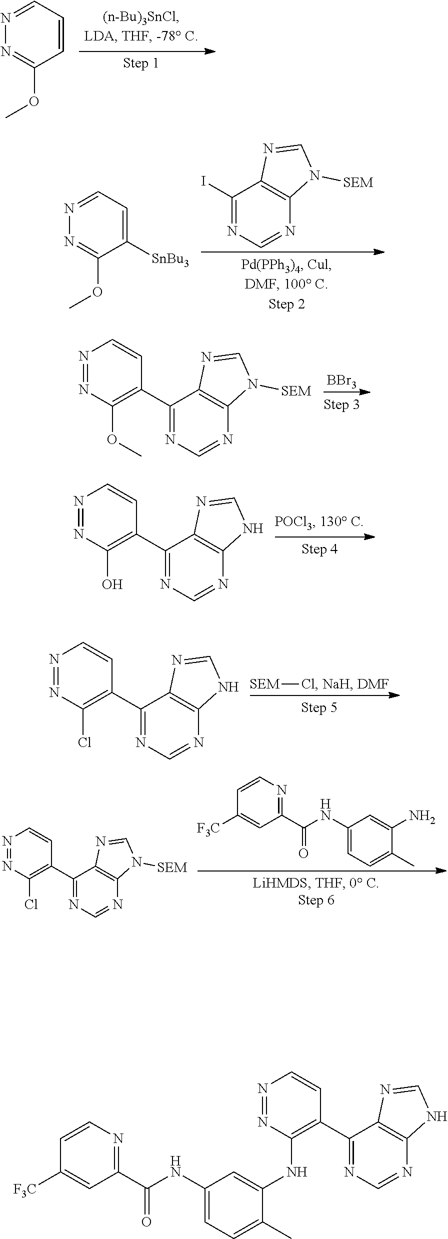

Step 1. To a mixture of diisopropylamine (2.42 g, 24 mmol) in tetrahydrofuran (40 mL) was added n-butyllithium (9.6 mL, 24 mmol) at −78° C. After stirring at −78° C. for 0.5 hour, 3-methoxypyridazine (2.2 g, 20 mmol) was added, and the mixture was stirred at −78° C. for 1 hour. Tributylchlorostannane (6 mL, 22 mmol) was added to the above mixture. After stirring at −78° C. for 1 hour, the mixture was quenched with a saturated ammonium chloride solution (30 mL) and extracted with ethyl acetate (40 mL×3). The organic phases were concentrated and purified by flash chromatography (petroleum ether/ethyl acetate=9/1) to afford 3-methoxy-4-(tributylstannyl)pyridazine (0.76 g, 1.9 mmol, 9.5%) as a yellow oil. MS (ESI) m/z 401.1 [M+H]⁺

Step 2. A mixture of 3-methoxy-4-(tributylstannyl)pyridazine (0.76 g, 1.9 mmol), copper(I) iodide (26 mg, 0.14 mmol), tetrakis(triphenylphosphine)palladium (160 mg, 0.14 mmol) and 6-iodo-9-((2-(trimethylsilyl)ethoxy)methyl)-9H-purine (0.55 g, 1.46 mmol) in N,N-dimethylformamide (2 mL) was stirred at 100° C. for 16 hours. After cooling to room temperature, the reaction mixture was poured into water (10 mL) and extracted with ethyl acetate (20 mL×3). The combined organic phases were concentrated and purified by flash chromatography (dichloromethane/methanol=10/1) to t afford 6-(3-methoxypyridazin-4-yl)-9-((2-(trimethylsilyl)ethoxy)methyl)-9H-purine (570 mg, 1.6 mmol, 84%) as a yellow solid. MS (ESI) m/z 358.5 [M+H]⁺

Step 3. A mixture of 6-(3-methoxypyridazin-4-yl)-9-((2-(trimethylsilyl)ethoxy)methyl)-9H-purine (570 mg, 1.6 mmol) in tribromoborane (17% in dichloromethane, 20 mL) was stirred at 20° C. for 3 days. The mixture was concentrated in vacuo to obtain 4-(9H-purin-6-yl)pyridazin-3-ol (0.6 g, crude) as a yellow solid. MS (ESI) m/z 215.1 [M+H]⁺

Step 4. A solution of 4-(9H-purin-6-yl)pyridazin-3-ol (0.6 g, crude) in phosphoryl trichloride (20 mL) was stirred at 130° C. for 1 hour and the mixture was concentrated in vacuo. The residue was poured into water (40 mL), the solid was collected by filtration and purified by prep-HPLC (Column: Welch Xtimate 21.2×250 mm C18, 10 μm, Mobile Phase: A: water (10 mmol/L trifluoroacetic acid) B: acetonitrile; B %: 0%-10% in 15 min) to afford 6-(3-chloropyridazin-4-yl)-9H-purine (0.2 g, 0.86 mmol) as a yellow solid. MS (ESI) m/z 233.1 [M+H]⁺

Step 5. A mixture of 6-(3-chloropyridazin-4-yl)-9H-purine (180 mg, 0.77 mmol) and sodium hydride (37 mg, 1.54 mmol) in N,N-dimethylformamide (5 mL) was stirred at 0° C. for 0.5 hour. (2-(chloromethoxy)ethyl)trimethylsilane (141 mg, 0.85 mmol) was added to the above mixture. After stirring at 0° C. for 1 hour, the mixture was poured into water (10 mL) and extracted with ethyl acetate (20 mL×3). The combined organic phases were concentrated and purified by flash chromatography (dichloromethane/methanol=10/1) to afford 6-(3-chloropyridazin-4-yl)-9-((2-(trimethylsilyl)ethoxy)methyl)-9H-purine (120 mg, 0.33 mmol, 43%) as a yellow oil. MS (ESI) m/z 363.3 [M+H]⁺

Step 6. To a solution of 6-(3-chloropyridazin-4-yl)-9-((2-(trimethylsilyl)ethoxy)methyl)-9H-purine (120 mg, 0.33 mmol) and N-(3-amino-4-methylphenyl)-4-(trifluoromethyl)picolinamide (98 mg, 0.33 mmol) in tetrahydrofuran (6 mL) was added lithium hexamethyldisilazide (2 mL, 3.3 mmol) at 0° C. After stirring at 0° C. for 1 hour, the mixture was quenched with saturated ammonium chloride solution (10 mL) and extracted with ethyl acetate (20 mL×3). The organic phases were concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((4-(9H-purin-6-yl)pyridazin-3-yl)amino)-4-methylphenyl)-4-(trifluoromethyl)picolinamide (9.8 mg, 0.02 mmol, 6%) as a yellow solid. ¹H NMR (500 MHZ, DMSO-d₆) δ 12.01 (s, 1H), 10.72 (s, 1H), 9.40 (d, J=4.9 Hz, 1H), 9.17 (s, 1H), 9.03 (dd, J=12.6, 5.0 Hz, 2H), 8.90-8.73 (m, 2H), 8.35 (s, 1H), 8.10 (d, J=5.0 Hz, 1H), 7.64 (dd, J=8.2, 2.0 Hz, 1H), 7.30 (d, J=8.4 Hz, 1H), 2.46 (s, 3H). MS (ESI) m/z 492.1 [M+H]⁺

### Example 119. N-(3-(3-(8-methoxy-9H-purin-6-yl)pyrazin-2-ylamino)-4-methylphenyl)-4-(trifluoromethyl)picolinamide (Compound IA-122)

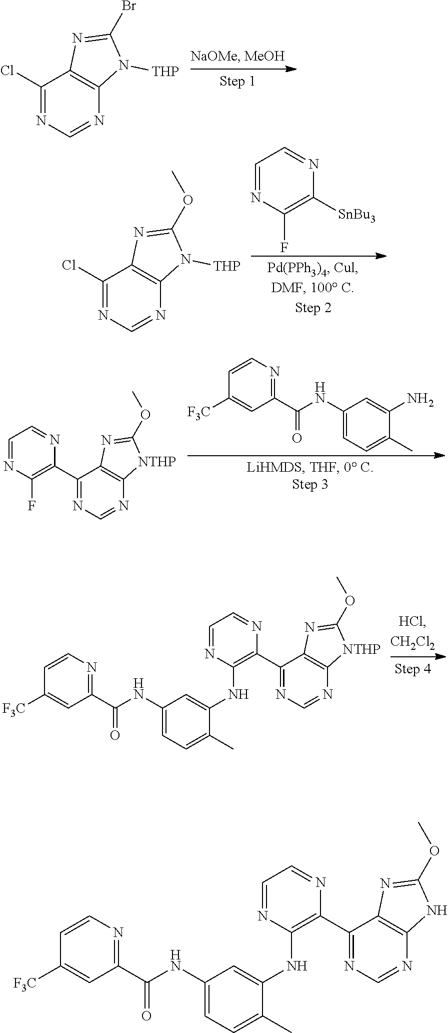

Step 1. A mixture of 8-bromo-6-chloro-9-(tetrahydro-2H-pyran-2-yl)-9H-purine (632 mg, 2 mmol) and sodium methoxide (864 mg, 4 mmol, 25% in methanol) in methanol (5 mL) was stirred at room temperature for 1 hour. The reaction mixture was concentrated and the residue was purified by flash chromatography (petroleum ether/ethyl acetate=6/4) to afford 6-chloro-8-methoxy-9-(tetrahydro-2H-pyran-2-yl)-9H-purine (500 mg, 1.86 mmol, 93%) as a colorless oil. MS (ESI) m/z 269.1 [M+H]⁺

Step 2. A mixture of 6-chloro-8-methoxy-9-(tetrahydro-2H-pyran-2-yl)-9H-purine (500 mg, 1.87 mmol), 2-fluoro-3-(tributylstannyl)pyrazine (869 mg, 2.24 mmol), tetrakis(triphenylphosphine)palladium (215 mg, 0.19 mmol) and cuprous iodide (35 mg, 0.19 mmol) in N,N-dimethylformamide (8 mL) was stirred at 100° C. for 18 hours. The reaction was treated with water (100 mL), extracted with ethyl acetate (30 mL×3) and the combined organic layers were concentrated. The residue was purified by flash chromatography (petroleum ether/ethyl acetate=1/1) to afford 6-(3-fluoropyrazin-2-yl)-8-methoxy-9-(tetrahydro-2H-pyran-2-yl)-9H-purine (300 mg, 0.91 mmol, 49%) as a yellow oil. MS (ESI) m/z 331.0 [M+H]⁺

Step 3. To a solution of 6-(3-fluoropyrazin-2-yl)-8-methoxy-9-(tetrahydro-2H-pyran-2-yl)-9H-purine (270 mg, 0.82 mmol) and N-(3-amino-4-methylphenyl)-4-(trifluoromethyl)picolinamide (241 mg, 0.82 mmol) in tetrahydrofuran (15 mL) was added lithium hexamethyldisilazide (2.55 mL, 4.09 mmol, 1.6 M) at 0° C. The mixture was stirred at room temperature for 1 hour, treated with saturated ammonium chloride solution (30 mL) and extracted with ethyl acetate (20 mL×2). The combined organic layers were concentrated and the residue was purified by flash chromatography (petroleum ether/ethyl acetate=1/1) to afford N-(3-((3-(8-methoxy-9-(tetrahydro-2H-pyran-2-yl)-9H-purin-6-yl)pyrazin-2-yl)amino)-4-methylphenyl)-4-(trifluoromethyl)picolinamide (45 mg, 0.074 mmol, 9%) as a yellow solid. MS (ESI) m/z 606.0 [M+H]⁺

Step 4. To a solution of N-(3-((3-(8-methoxy-9-(tetrahydro-2H-pyran-2-yl)-9H-purin-6-yl)pyrazin-2-yl)amino)-4-methylphenyl)-4-(trifluoromethyl)picolinamide (45 mg, 0.07 mmol) in dichloromethane (1 mL) was added hydrogen chloride (0.1 mL, 3 M in methanol). The mixture was stirred at room temperature for 1 hour. The reaction mixture was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(8-methoxy-9H-purin-6-yl)pyrazin-2-yl)amino)-4-methylphenyl)-4-(trifluoromethyl)picolinamide (15 mg, 0.029 mmol, 41%) as a yellow solid. ¹H NMR (400 MHZ, DMSO-d₆) δ 10.69 (s, 1H), 10.48 (s, 1H), 9.04 (d, J=5.0 Hz, 1H), 8.69-8.54 (m, 2H), 8.35 (s, 1H), 8.27 (d, J=2.5 Hz, 1H), 8.18 (d, J=2.5 Hz, 1H), 8.09 (d, J=3.9 Hz, 1H), 7.54 (dd, J=8.2, 2.0 Hz, 1H), 7.23 (d, J=8.4 Hz, 1H), 3.26 (s, 3H), 2.30 (s, 3H). MS (ESI) m/z 521.7 [M+H]⁺

### Example 120. N-(3-(3-(8-methoxy-9H-purin-6-yl)pyridin-2-ylamino)-4-methylphenyl)-4-(trifluoromethyl)picolinamide (Compound IA-123)

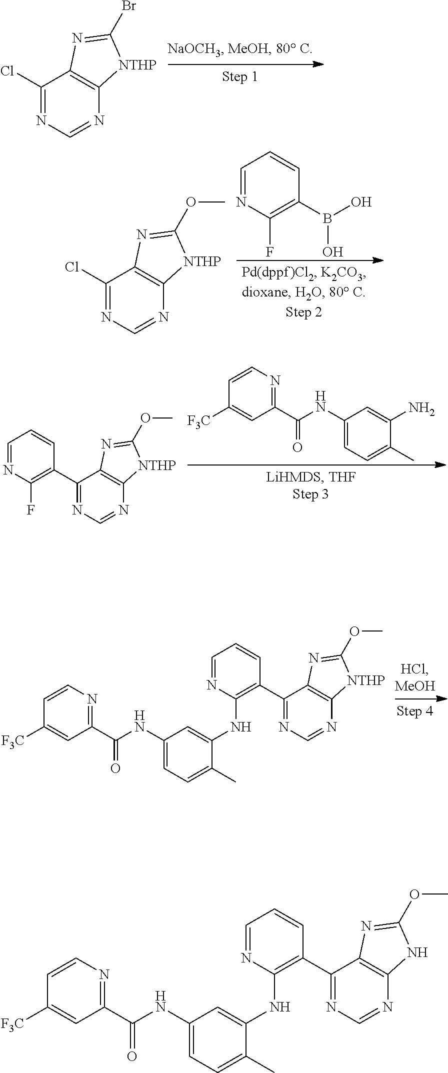

Step 1. The mixture of 8-bromo-6-chloro-9-(tetrahydro-2H-pyran-2-yl)-9H-purine (500 mg, 1.58 mmol) and sodium methoxide (684 mg, 3.16 mmol, 25% in methanol) in methanol (3 mL) was stirred at 80° C. for 18 hours. The reaction mixture was concentrated and the residue was purified by flash chromatography (petroleum ether/ethyl acetate=6/4) to afford 6-chloro-8-methoxy-9-(tetrahydro-2H-pyran-2-yl)-9H-purine (410 mg, 1.53 mmol, 97%) as a yellow oil. MS (ESI) m/z 269.1[M+H]⁺

Step 2. The mixture of 6-chloro-8-methoxy-9-(tetrahydro-2H-pyran-2-yl)-9H-purine (410 mg, 1.53 mmol), (2-fluoropyridin-3-yl)boronic acid (237 mg, 1.68 mmol), 1,1′-bis(diphenylphosphino)ferrocene-palladium(II)dichloride dichloromethane complex (125 mg, 0.15 mmol) and potassium carbonate (422 mg, 3.06 mmol) in dioxane (6 mL) and water (2 mL) was stirred at 80° C. for 1 hour. The reaction mixture was concentrated and the residue was purified by flash chromatography (petroleum ether/ethyl acetate=6/4) to afford 6-(2-fluoropyridin-3-yl)-8-methoxy-9-(tetrahydro-2H-pyran-2-yl)-9H-purine (350 mg, 1.06 mmol, 70%) as a yellow solid. MS (ESI) m/z 330.0 [M+H]⁺

Step 3. To a solution of 6-(2-fluoropyridin-3-yl)-8-methoxy-9-(tetrahydro-2H-pyran-2-yl)-9H-purine (130 mg, 0.4 mmol) and N-(3-amino-4-methylphenyl)-4-(trifluoromethyl)picolinamide (117 mg, 0.4 mmol) in tetrahydrofuran (10 mL) was added lithium hexamethyldisilazide (1.24 mL, 2 mmol, 1.6 M) at 0° C. The mixture was stirred at room temperature for 1 hour. The reaction was treated with saturated ammonium chloride solution (50 mL), extracted with ethyl acetate (30 mL×2) and the combined organic layers were concentrated. The residue was purified by flash chromatography (petroleum ether/ethyl acetate=1/1) to afford N-(3-((3-(8-methoxy-9-(tetrahydro-2H-pyran-2-yl)-9H-purin-6-yl)pyridin-2-yl)amino)-4-methylphenyl)-4-(trifluoromethyl)picolinamide (130 mg, 0.22 mmol, 54%) as a yellow solid. MS (ESI) m/z 605.1 [M+H]⁺

Step 4. To a solution of N-(3-((3-(8-methoxy-9-(tetrahydro-2H-pyran-2-yl)-9H-purin-6-yl)pyridin-2-yl)amino)-4-methylphenyl)-4-(trifluoromethyl)picolinamide (130 mg, 0.22 mmol) in methanol (1 mL) was added hydrochloric acid (0.5 mL, 3 M in methanol). The mixture was stirred at room temperature for 1 hour and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(8-methoxy-9H-purin-6-yl)pyridin-2-yl)amino)-4-methylphenyl)-4-(trifluoromethyl)picolinamide (5 mg, 0.01 mmol, 4.4%) as a yellow solid ¹H NMR (400 MHZ, DMSO-d₆) δ 12.35 (s, 1H), 10.63 (s, 1H), 9.53 (s, 1H), 9.04 (d, J=4.8 Hz, 1H), 8.83 (d, J=7.6 Hz, 2H), 8.33 (d, J=20.5 Hz, 2H), 8.09 (d, J=5.0 Hz, 1H), 7.52 (d, J=8.3 Hz, 1H), 7.24 (d, J=8.5 Hz, 1H), 7.08-6.94 (m, 1H), 4.18 (s, 3H), 2.43 (s, 3H). MS (ESI) m/z 520.7 [M+H]⁺

### Example 121. N-(3-(3-(8-(2-(dimethylamino)ethoxy)-9H-purin-6-yl)pyridin-2-ylamino)-4-methylphenyl)-4-(trifluoromethyl)picolinamide (Compound IA-124)

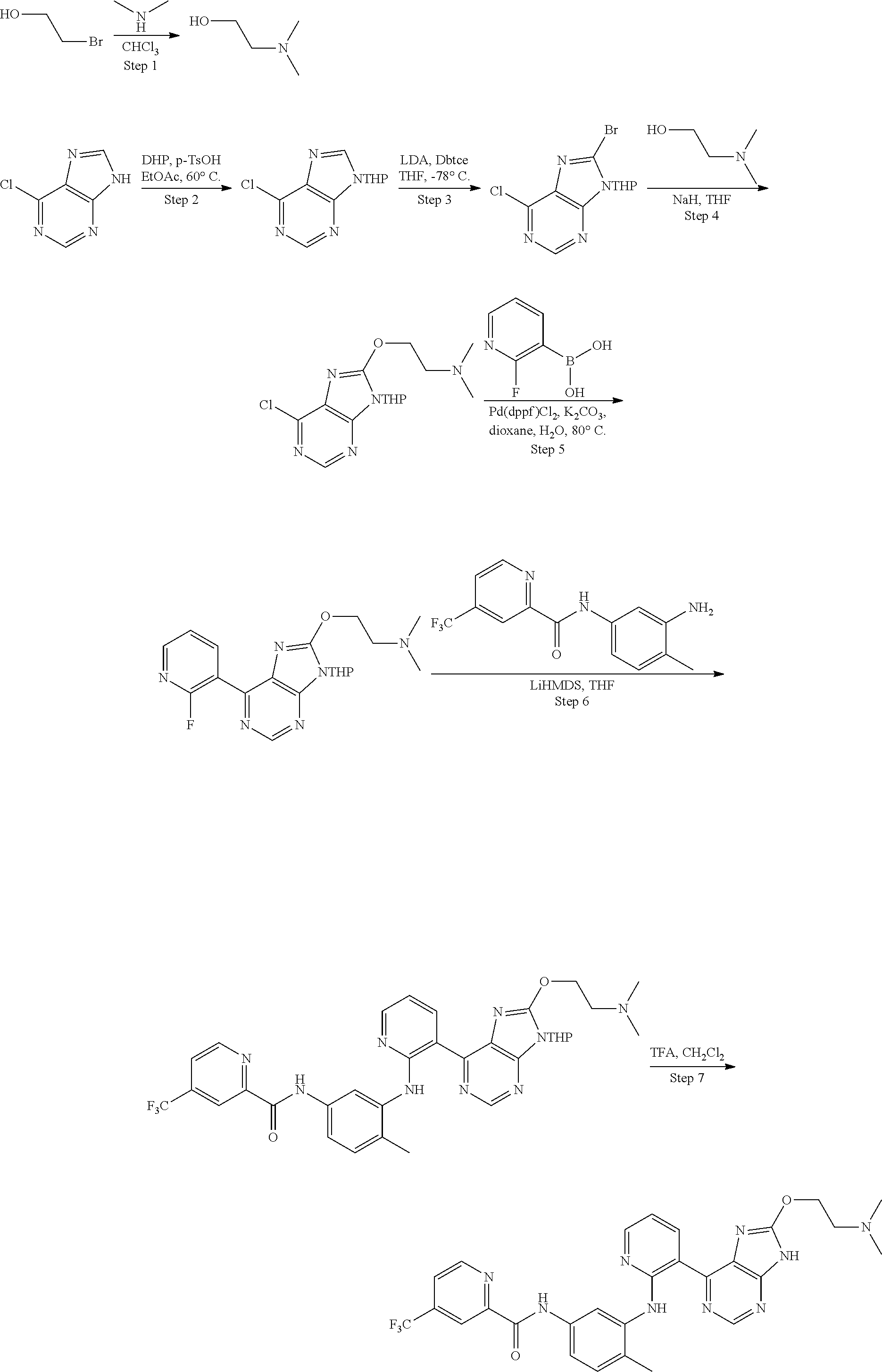

Step 1. A mixture of 2-bromoethan-1-ol (2.5 g, 20 mmol) and dimethylamine (13 mL, 26 mmol, 2 M in tetrahydrofuran) in chloroform (10 mL) was stirred at room temperature for 18 hours. The reaction mixture was concentrated. The residue was treated with water (50 mL) and dichloromethane (50 mL). The organic layer was separated and discarded. The aqueous was concentrated. The residue was dissolved in dichloromethane/methanol (20 mL, 10/1) and stirred for 1 hour. The mixture was filtered and washed with dichloromethane/methanol (5 mL, 10/1). The filtrate was concentrated to afford 2-(dimethylamino)ethan-1-ol (500 mg, 5.6 mmol, 28%) as a yellow oil. ¹H NMR (400 MHZ, CDCl₃) δ 4.07-3.92 (m, 2H), 3.26-3.17 (m, 2H), 2.91 (d, J=21.1 Hz, 6H).

Step 2. A mixture of 6-chloro-9H-purine (3 g, 19.48 mmol), 3,4-dihydro-2H-pyran (4.9 g, 58.44 mmol) and p-toluenesulfonic acid (370 mg, 1.95 mmol) in ethyl acetate (60 mL) was stirred at 60° C. for 2 hours. The reaction mixture was concentrated and purified by flash chromatography (petroleum ether/ethyl acetate=1/1) to afford 6-chloro-9-(tetrahydro-2H-pyran-2-yl)-9H-purine (4.1 g, 17.2 mmol, 88%) as a yellow oil. MS (ESI) m/z 239.0 [M+H]⁺

Step 3. To a solution of diisopropylamine (1.65 g, 16.39 mmol) in tetrahydrofuran (10 mL) was added n-butyllithium (6.56 mL, 16.39 mmol, 2.5 M) at −78° C. The mixture was stirred at −78° C. for 20 minutes, and 6-chloro-9-(tetrahydro-2H-pyran-2-yl)-9H-purine (3.0 g, 12.66 mmol) in tetrahydrofuran (10 mL) was added. The reaction mixture was stirred at −78° C. for 30 minutes. A solution of 1,2-dibromotetrachloroethane (6.15 g, 18.91 mmol) in tetrahydrofuran (10 mL) was added and stirred at −78° C. for 1 hour. The reaction was treated with saturated ammonium chloride solution (200 mL) and extracted with ethyl acetate (100 mL×2). The combined organic layers were concentrated. The residue was purified by flash chromatography (petroleum ether/ethyl acetate=3/1) to afford 8-bromo-6-chloro-9-(tetrahydro-2H-pyran-2-yl)-9H-purine (2.8 g, 8.86 mmol, 70%) as a yellow solid. MS (ESI) m/z 232.9 [M+H]⁺

Step 4. To a solution of 2-(dimethylamino)ethan-1-ol (211 mg, 2.37 mmol) in tetrahydrofuran (10 mL) was added sodium hydride (127 mg, 3.16 mmol, 60%) at 0° C. The mixture was stirred at room temperature for 0.5 hour. 8-Bromo-6-chloro-9-(tetrahydro-2H-pyran-2-yl)-9H-purine (500 mg, 1.58 mmol) was added and the mixture was stirred at room temperature for 1 hour. The reaction mixture was quenched with methanol (2 mL) and concentrated. The residue was purified by flash chromatography (dichloromethane/methanol=5/1) to afford 2-((6-chloro-9-(tetrahydro-2H-pyran-2-yl)-9H-purin-8-yl)oxy)-N,N-dimethylethan-1-amine (300 mg, 0.92 mmol, 58%) as a yellow oil. MS (ESI) m/z 326.1 [M+H]⁺

Step 5. The mixture of 2-((6-chloro-9-(tetrahydro-2H-pyran-2-yl)-9H-purin-8-yl)oxy)-N,N-dimethylethan-1-amine (300 mg, 0.92 mmol), (2-fluoropyridin-3-yl)boronic acid (143 mg, 1.02 mmol), 1,1′-bis(diphenylphosphino)ferrocene-palladium(II)dichloride dichloromethane complex (75 mg, 0.09 mmol) and potassium carbonate (255 mg, 1.85 mmol) in dioxane (6 mL) and water (2 mL) was stirred at 80° C. for 1 hour under nitrogen. The reaction mixture was concentrated and the residue was purified by flash chromatography (dichloromethane/methanol=10/1) to afford 2-((6-(2-fluoropyridin-3-yl)-9-(tetrahydro-2H-pyran-2-yl)-9H-purin-8-yl)oxy)-N,N-dimethylethan-1-amine (240 mg, 0.62 mmol, 68%) as a yellow oil. MS (ESI) m/z 387.0 [M+H]⁺

Step 6. To a solution of 2-((6-(2-fluoropyridin-3-yl)-9-(tetrahydro-2H-pyran-2-yl)-9H-purin-8-yl)oxy)-N,N-dimethylethan-1-amine (210 mg, 0.54 mmol) and N-(3-amino-4-methylphenyl)-4-(trifluoromethyl)picolinamide (160 mg, 0.54 mmol) in tetrahydrofuran (5 mL) was added lithium hexamethyldisilazide (1.7 mL, 2.72 mmol, 1.6 M) at 0° C. and the mixture was stirred at room temperature for 1 hour. The reaction was treated with saturated ammonium chloride solution (50 mL), extracted with ethyl acetate (50 mL×2) and the combined organic layers were concentrated. The residue was purified by flash chromatography (dichloromethane/methanol=20/1) to afford N-(3-((3-(8-(2-(dimethylamino)ethoxy)-9-(tetrahydro-2H-pyran-2-yl)-9H-purin-6-yl)pyridin-2-yl)amino)-4-methylphenyl)-4-(trifluoromethyl)picolinamide (135 mg, 0.2 mmol, 38%) as a yellow oil. MS (ESI) m/z 662.2 [M+H]⁺

Step 7. To a solution of N-(3-((3-(8-(2-(dimethylamino)ethoxy)-9-(tetrahydro-2H-pyran-2-yl)-9H-purin-6-yl)pyridin-2-yl)amino)-4-methylphenyl)-4-(trifluoromethyl)picolinamide (130 mg, 0.2 mmol) in dichloromethane (0.5 mL) was added trifluoroacetic acid (0.5 mL) and the mixture was stirred at room temperature for 1 hour. The reaction mixture was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(8-(2-(dimethylamino)ethoxy)-9H-purin-6-yl)pyridin-2-yl)amino)-4-methylphenyl)-4-(trifluoromethyl)picolinamide (33.5 mg, 0.058 mmol, 29%) as a yellow solid. ¹H NMR (400 MHZ, DMSO-d₆) δ 12.31 (s, 1H), 10.62 (s, 1H), 9.53-9.41 (m, 1H), 9.04 (d, J=5.1 Hz, 1H), 8.91-8.78 (m, 2H), 8.36 (s, 1H), 8.30 (dd, J=4.6, 1.8 Hz, 1H), 8.09 (d, J=3.9 Hz, 1H), 7.56-7.46 (m, 1H), 7.24 (d, J=8.4 Hz, 1H), 6.99 (dd, J=7.8, 4.7 Hz, 1H), 4.70 (t, J=5.3 Hz, 2H), 2.87 (t, J=5.3 Hz, 2H), 2.42 (s, 3H), 2.36 (s, 6H). MS (ESI) m/z 578.0 [M+H]⁺

### Example 122. N-(3-(3-(8-(2-hydroxyethoxy)-9H-purin-6-yl)pyridin-2-ylamino)-4-methylphenyl)-4-(trifluoromethyl)picolinamide (Compound IA-125)

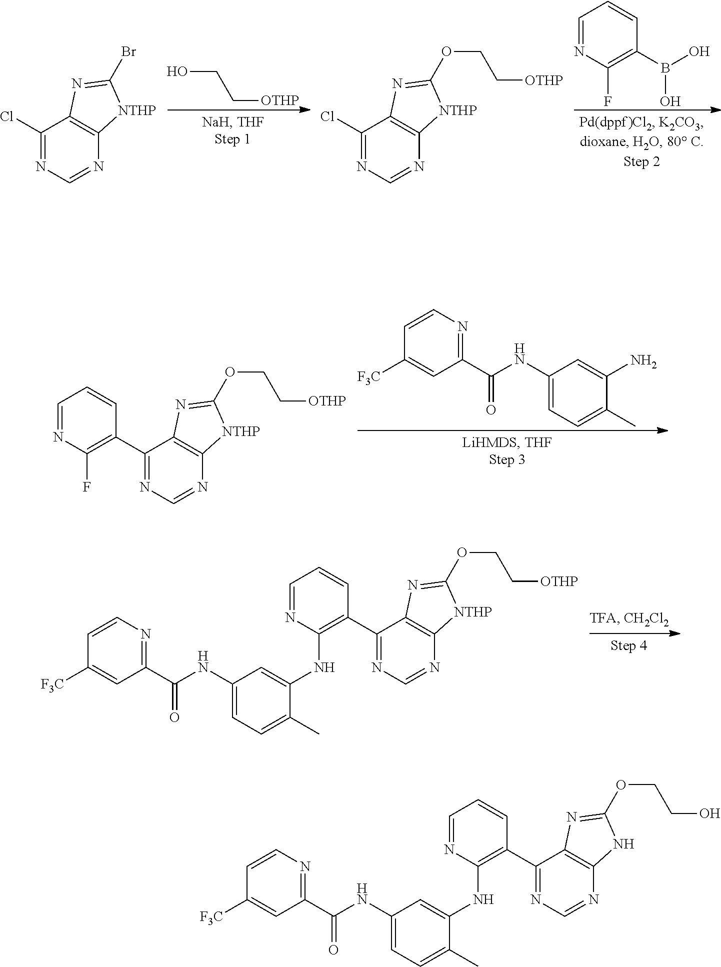

Step 1. To a solution of 2-((tetrahydro-2H-pyran-2-yl)oxy)ethan-1-ol (346 mg, 2.37 mmol) in tetrahydrofuran (10 mL) was added sodium hydride (127 mg, 3.16 mmol, 60%) at 0° C. The mixture was stirred at room temperature for 0.5 hour and 8-bromo-6-chloro-9-(tetrahydro-2H-pyran-2-yl)-9H-purine (500 mg, 1.58 mmol) was added. The reaction mixture was stirred for 1 hour, then quenched with methanol (2 mL) and concentrated. The residue was purified by flash chromatography (petroleum ether/ethyl acetate=1/1) to afford 6-chloro-9-(tetrahydro-2H-pyran-2-yl)-8-(2-((tetrahydro-2H-pyran-2-yl)oxy)ethoxy)-9H-purine (150 mg, 0.39 mmol, 25%) as a yellow oil. MS (ESI) m/z 382.8 [M+H]⁺

Step 2. A mixture of 6-chloro-9-(tetrahydro-2H-pyran-2-yl)-8-(2-((tetrahydro-2H-pyran-2-yl)oxy)ethoxy)-9H-purine (150 mg, 0.39 mmol), (2-fluoropyridin-3-yl)boronic acid (61 mg, 0.43 mmol), 1,1′-bis(diphenylphosphino)ferrocene-palladium(II)dichloride dichloromethane complex (32 mg, 0.04 mmol) and potassium carbonate (108 mg, 0.79 mmol) in dioxane (5 mL) and water (1.5 mL) was stirred at 80° C. for 1 hour under nitrogen. The reaction mixture was concentrated and the residue was purified by flash chromatography (petroleum ether/ethyl acetate=1/1) to afford 6-(2-fluoropyridin-3-yl)-9-(tetrahydro-2H-pyran-2-yl)-8-(2-((tetrahydro-2H-pyran-2-yl)oxy)ethoxy)-9H-purine (130 mg, 0.29 mmol, 75%) as a yellow oil. MS (ESI) m/z 444.2 [M+H]⁺

Step 3. To a solution of 6-(2-fluoropyridin-3-yl)-9-(tetrahydro-2H-pyran-2-yl)-8-(2-((tetrahydro-2H-pyran-2-yl)oxy)ethoxy)-9H-purine (130 mg, 0.29 mmol) and N-(3-amino-4-methylphenyl)-4-(trifluoromethyl)picolinamide (87 mg, 0.29 mmol) in tetrahydrofuran (10 mL) was added lithium hexamethyldisilazide (0.92 mL, 1.47 mmol, 1.6 M) at 0° C. The mixture was stirred at room temperature for 1 hour, quenched with saturated ammonium chloride solution (30 mL), and extracted with ethyl acetate (30 mL×2). The combined organic layers were concentrated and the residue was purified by flash chromatography (petroleum ether/ethyl acetate=1/1) to N-(4-methyl-3-((3-(9-(tetrahydro-2H-pyran-2-yl)-8-(2-((tetrahydro-2H-pyran-2-afford yl)oxy)ethoxy)-9H-purin-6-yl)pyridin-2-yl)amino)phenyl)-4-(trifluoromethyl)picolinamide (115 mg, 0.16 mmol, 55%) as a yellow solid. MS (ESI) m/z 719.1 [M+H]⁺

Step 4. To a solution of N-(4-methyl-3-((3-(9-(tetrahydro-2H-pyran-2-yl)-8-(2-((tetrahydro-2H-pyran-2-yl)oxy)ethoxy)-9H-purin-6-yl)pyridin-2-yl)amino)phenyl)-4-(trifluoromethyl)-picolinamide (50 mg, 0.07 mmol) in dichloromethane (0.5 mL) was added trifluoroacetic acid (0.3 mL). The mixture was stirred at room temperature for 5 hours and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(8-(2-hydroxyethoxy)-9H-purin-6-yl)pyridin-2-yl)amino)-4-methylphenyl)-4-(trifluoromethyl)picolinamide (11.4 mg, 0.02 mmol, 30%) as a yellow solid. ¹H NMR (400 MHZ, DMSO-d₆) δ 13.42 (s, 1H), 12.26 (s, 1H), 10.64 (s, 1H), 9.53 (s, 1H), 9.04 (d, J=4.9 Hz, 1H), 8.89 (s, 1H), 8.83 (s, 1H), 8.47-8.23 (m, 2H), 8.10 (d, J=3.9 Hz, 1H), 7.52 (dd, J=8.1, 1.9 Hz, 1H), 7.24 (d, J=8.2 Hz, 1H), 7.01 (dd, J=7.8, 4.7 Hz, 1H), 5.04 (s, 1H), 4.72-4.56 (m, 2H), 3.83 (s, 2H), 2.42 (s, 3H). MS (ESI) m/z 550.9 [M+H]⁺

### Example 123. N-(4-methyl-3-((3-(8-methyl-9H-purin-6-yl)pyridin-2-yl)amino)phenyl)-4-(trifluoromethyl)picolinamide (Compound IA-126)

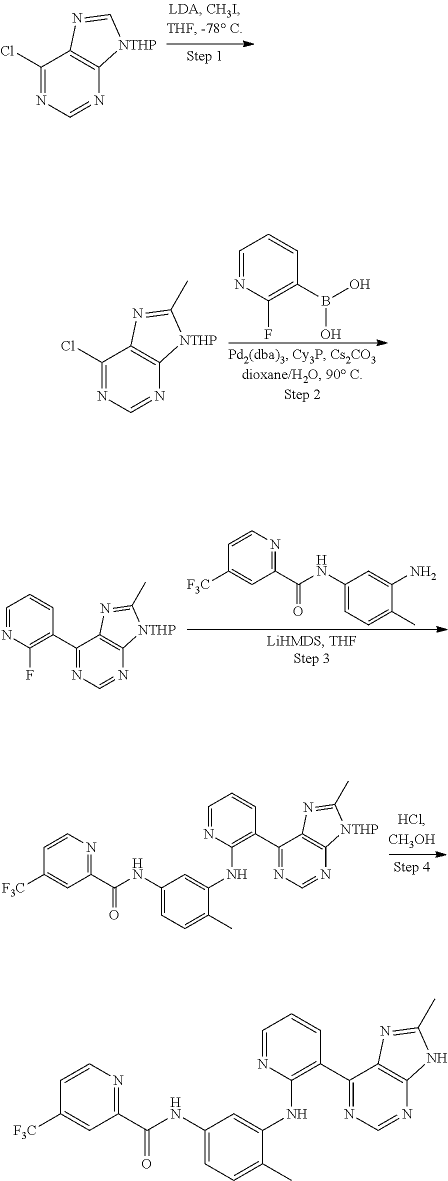

Step 1. To a solution of 6-chloro-9-(tetrahydro-2H-pyran-2-yl)-9H-purine (1 g, 4.2 mmol) in tetrahydrofuran (15 mL) was added lithium diisopropylamide (630 mg, 5.88 mmol) at −78° C. The mixture was stirred at −78° C. for 1 hour, then iodomethane (6.0 g, 42 mmol) was added and the mixture was stirred at −78° C. for another 16 hours. The mixture was washed with water (30 mL) and extracted with ethyl acetate (30 mL×3). The combined organic phases were dried over sodium sulfate, filtered and concentrated under reduced pressure to afford 6-chloro-8-methyl-9-(tetrahydro-2H-pyran-2-yl)-9H-purine (1.2 g, 4.76 mmol, 81%) as a yellow oil. MS (ESI) m/z 169.1 [M+H]⁺

Step 2. A mixture of 6-chloro-8-methyl-9-(tetrahydro-2H-pyran-2-yl)-9H-purine (500 mg, 1.98 mmol), (2-fluoropyridin-3-yl)boronic acid (280 mg, 1.98 mmol), tris(dibenzylideneacetone)dipalladium (183 mg, 0.2 mmol), tricyclohexyl phosphine (112 mg, 0.4 mmol) and cesium carbonate (1.9 g, 5.94 mmol) in 1,4-dioxane (20 mL) and water (2 mL) was stirred at 90° C. for 16 hours under nitrogen. The mixture was diluted with ethyl acetate (15 mL) and washed with water (10 mL). The organic phase was separated, and the aqueous phase was extracted with ethyl acetate (15 mL×2). The combined organic layers were dried over anhydrous sodium sulfate, filtered, and concentrated by rotary evaporation. The crude mixture was purified by flash chromatography (100% ethyl acetate) to afford 6-(2-fluoropyridin-3-yl)-8-methyl-9-(tetrahydro-2H-pyran-2-yl)-9H-purine (540 mg, 1.72 mmol, 87%) as a yellow solid. MS (ESI) m/z 314.1 [M+H]⁺

Step 3. To a solution of 6-(2-fluoropyridin-3-yl)-8-methyl-9-(tetrahydro-2H-pyran-2-yl)-9H-purine (200 mg, 0.64 mmol) and N-(3-amino-4-methylphenyl)-4-(trifluoromethyl)picolinamide (189 mg, 0.64 mmol) in tetrahydrofuran (10 mL) was slowly added lithium hexamethyldisilazide (1.6 mol/L in tetrahydrofuran, 1.2 mL, 1.92 mmol) at 0° C. and the mixture was stirred at 0° C. for 1 hour. The crude residue was purified by flash chromatography (10% methanol in dichloromethane) to afford N-(4-methyl-3-((3-(8-methyl-9-(tetrahydro-2H-pyran-2-yl)-9H-purin-6-yl)pyridin-2-yl)amino)phenyl)-4-(trifluoromethyl)picolinamide (94 mg, 0.15 mmol, 25%) as a yellow oil. MS (ESI) m/z 589.3 [M+H]⁺

Step 4. A mixture of N-(4-methyl-3-((3-(8-methyl-9-(tetrahydro-2H-pyran-2-yl)-9H-purin-6-yl)pyridin-2-yl)amino)phenyl)-4-(trifluoromethyl)picolinamide (94 mg, 0.15 mmol) in hydrochloric acid (3 M in methanol, 5 mL) was stirred at room temperature for 2 hours. The reaction mixture was concentrated, and then the pH was adjusted to alkaline with ammonia (7 M in methanol). The resulting mixture was purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford N-(4-methyl-3-((3-(8-methyl-9H-purin-6-yl)pyridin-2-yl)amino)phenyl)-4-(trifluoromethyl)picolinamide (14.8 mg, 0.03 mmol, 19.6%) as a yellow solid. ¹H NMR (400 MHZ, DMSO-d₆) δ 13.59 (s, 1H), 12.36 (s, 1H), 10.64 (s, 1H), 9.78 (dd, J=7.9, 1.9 Hz, 1H), 9.04 (d, J=5.0 Hz, 1H), 8.99 (s, 1H), 8.84 (d, J=2.1 Hz, 1H), 8.35 (d, J=4.6 Hz, 2H), 8.09 (d, J=5.0 Hz, 1H), 7.56-7.51 (m, 1H), 7.26 (d, J=8.2 Hz, 1H), 7.04 (dd, J=7.8, 4.7 Hz, 1H), 2.65 (s, 3H), 2.44 (s, 3H). MS (ESI) m/z 504.7 [M+H]⁺

### Example 124. 2-(2-cyanopropan-2-yl)-N-(4-methyl-3-((3-(8-methyl-9H-purin-6-yl)pyridin-2-yl)amino)phenyl)isonicotinamide (Compound IA-127)

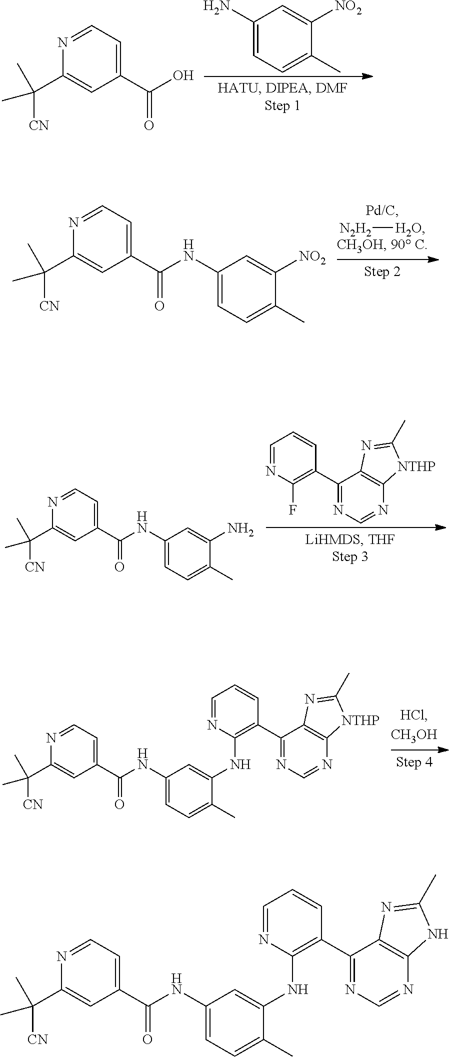

Step 1. A solution of 2-(2-cyanopropan-2-yl)isonicotinic acid (400 mg, 2.11 mmol), 4-methyl-3-nitroaniline (320 mg, 2.11 mmol), 1-[bis(dimethylamino)methylene]-1H-1,2,3-triazolo[4,5-b]pyridinium 3-oxid hexafluorophosphate (2.4 g, 6.33 mmol) and triethylamine (0.4 mL) in N,N-dimethylformamide (10 mL) was stirred at room temperature for 2 hours. The mixture was quenched with brine (30 mL) and extracted with ethyl acetate (30 mL×3). The combined organic phases were dried over sodium sulfate, filtered and concentrated under reduced pressure to afford 2-(2-cyanopropan-2-yl)-N-(4-methyl-3-nitrophenyl)isonicotinamide (596 mg, 1.84 mmol, 87.2%) as a yellow solid. MS (ESI) m/z 325.2 [M+H]⁺

Step 2. To a solution of 2-(2-cyanopropan-2-yl)-N-(4-methyl-3-nitrophenyl)isonicotinamide (596 mg, 1.84 mmol) in methanol (10 mL) was added hydrazine hydrate (46 mg, 0.92 mmol) and 10% palladium on activated carbon (60 mg). The reaction mixture was stirred at 70° C. for 1 hour, cooled to room temperature and the reaction mixture was concentrated. The residue was purified by flash chromatography (methanol/dichloromethane=1/9) to afford N-(3-amino-4-methylphenyl)-2-(2-cyanopropan-2-yl)isonicotinamide (487 mg, 1.66 mmol, 90%) as a yellow solid. MS (ESI) m/z 295.3 [M+H]⁺

Step 3. A solution of N-(3-amino-4-methylphenyl)-2-(2-cyanopropan-2-yl)isonicotinamide (70 mg, 0.24 mmol), 6-(2-fluoropyridin-3-yl)-8-methyl-9-(tetrahydro-2H-pyran-2-yl)-9H-purine (75 mg, 0.24 mmol) and lithium hexamethyldisilazide (200 mg, 1.2 mmol) in tetrahydrofuran (5 mL) was stirred at 0° C. for 1 hour under nitrogen atmosphere. The reaction mixture was quenched with water (30 mL) and extracted with ethyl acetate (30 mL×3). The combined organic layers were washed with brine (30 mL), dried over sodium sulfate, filtered and concentrated. The residue was purified flash chromatography (ethyl acetate) to afford 2-(2-cyanopropan-2-yl)-N-(4-methyl-3-((3-(8-methyl-9-(tetrahydro-2H-pyran-2-yl)-9H-purin-6-yl)pyridin-2-yl)amino)phenyl)isonicotinamide (119 mg, 0.2 mmol, 84%) as a yellow oil. MS (ESI) m/z 588.3 [M+H]⁺

Step 4. A solution of 2-(2-cyanopropan-2-yl)-N-(4-methyl-3-((3-(8-methyl-9-(tetrahydro-2H-pyran-2-yl)-9H-purin-6-yl)pyridin-2-yl)amino)phenyl)isonicotinamide (119 mg, 0.2 mmol) in hydrochloric acid (3 M in methanol, 3 mL) was stirred at room temperature for 1 hour. The reaction mixture was concentrated, and the pH was adjusted to alkaline with ammonia (7 M in methanol). The resulting mixture was purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford 2-(2-cyanopropan-2-yl)-N-(4-methyl-3-((3-(8-methyl-9H-purin-6-yl)pyridin-2-yl)amino)phenyl)isonicotinamide (6.1 mg, 0.01 mmol, 6%). ¹H NMR (500 MHZ, DMSO-d₆) δ 13.57 (s, 1H), 12.36 (s, 1H), 10.52 (s, 1H), 9.77 (s, 1H), 8.99 (s, 1H), 8.80 (d, J=5.1 Hz, 1H), 8.68 (s, 1H), 8.33 (dd, J=4.7, 1.9 Hz, 1H), 8.02 (s, 1H), 7.88 (dd, J=5.0, 1.4 Hz, 1H), 7.44 (d, J=8.0 Hz, 1H), 7.26 (d, J=8.2 Hz, 1H), 7.04 (dd, J=7.8, 4.7 Hz, 1H), 2.65 (s, 3H), 2.45 (s, 3H), 1.77 (s, 6H). MS (ESI) m/z 504.4 [M+H]⁺

### Example 125. 5-fluoro-N-(4-methyl-3-((3-(8-methyl-9H-purin-6-yl)pyridin-2-yl)amino)phenyl)-4-(trifluoromethyl)picolinamide (Compound IA-128)

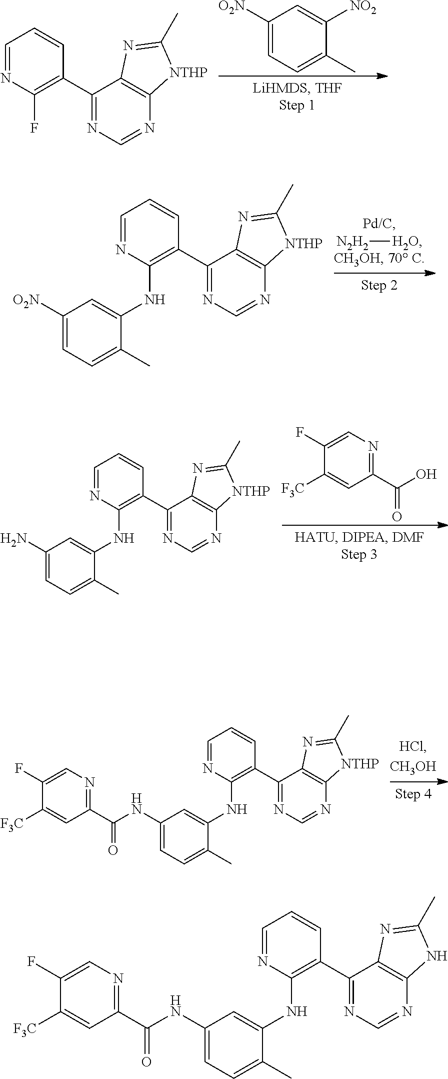

Step 1. A solution of 6-(2-fluoropyridin-3-yl)-8-methyl-9-(tetrahydro-2H-pyran-2-yl)-9H-purine (200 mg, 0.64 mmol), 2-methyl-5-nitroaniline (97 mg, 0.64 mmol) and lithium hexamethyldisilazide (534 mg, 3.2 mmol) in tetrahydrofuran (5 mL) was stirred at 0° C. for 1 hour under nitrogen. The reaction solution was quenched with water (30 mL) and extracted with ethyl acetate (50 mL). The organic layer was washed with brine (30 mL), dried over sodium sulfate, filtered and concentrated. The crude residue was purified by flash chromatography (petroleum ether/ethyl acetate=1/1) to afford N-(2-methyl-5-nitrophenyl)-3-(8-methyl-9-(tetrahydro-2H-pyran-2-yl)-9H-purin-6-yl)pyridin-2-amine (136 mg, 0.31 mmol, 47.8%) as a yellow oil. MS (ESI) m/z 446.2 [M+H]⁺

Step 2. To a solution of N-(2-methyl-5-nitrophenyl)-3-(8-methyl-9-(tetrahydro-2H-pyran-2-yl)-9H-purin-6-yl)pyridin-2-amine (136 mg, 0.31 mmol) in methanol (10 mL) was added hydrazine hydrate (8 mg, 0.15 mmol) and 10% palladium on carbon (14 mg). The mixture was stirred at 70° C. for 1 hour, cooled to room temperature, filtered through celite and concentrated. The residue was purified by flash chromatography (petroleum ether/ethyl acetate=1/2) to afford 6-methyl-N1-(3-(8-methyl-9-(tetrahydro-2H-pyran-2-yl)-9H-purin-6-yl)pyridin-2-yl)benzene-1,3-diamine (113 mg, 0.27 mmol, 87.8%) as a yellow oil. MS (ESI) m/z 415.8 [M+H]⁺

Step 3. A solution of 5-fluoro-4-(trifluoromethyl)picolinic acid (40 mg, 0.19 mmol), 6-methyl-N1-(3-(8-methyl-9-(tetrahydro-2H-pyran-2-yl)-9H-purin-6-yl)pyridin-2-yl)benzene-1,3-diamine (80 mg, 0.19 mmol), 1-[bis(dimethylamino)methylene]-1H-1,2,3-triazolo[4,5-b]pyridinium 3-oxid hexafluorophosphate (217 mg, 0.57 mmol) and triethylamine (0.2 mL) in N,N-dimethylformamide (3 mL) was stirred at room temperature for 2 hours. The reaction solution was purified flash chromatography (petroleum ether/ethyl acetate=1/3) to afford 5-fluoro-N-(4-methyl-3-((3-(8-methyl-9-(tetrahydro-2H-pyran-2-yl)-9H-purin-6-yl)pyridin-2-yl)amino)phenyl)-4-(trifluoromethyl)picolinamide (72 mg, 0.12 mmol, 62.5%) as a brown oil. MS (ESI) m/z 607.2 [M+H]⁺

Step 4. A solution of 5-fluoro-N-(4-methyl-3-((3-(8-methyl-9-(tetrahydro-2H-pyran-2-yl)-9H-purin-6-yl)pyridin-2-yl)amino)phenyl)-4-(trifluoromethyl)picolinamide (72 mg, 0.12 mmol) in hydrochloric acid (3 M in methanol, 5 mL) was stirred at room temperature for 2 hours. The reaction mixture was concentrated, and the pH was adjusted to alkaline with ammonia (7 M in methanol). The resulting mixture was purified by prep-HPLC (column: Welch Xtimate 21.2× 250 mm C18, 10 μm, Mobile Phase: A: water (10 mmol/L ammonium bicarbonate) B: acetonitrile; B %: 30%-70% in 15 min) to afford 5-fluoro-N-(4-methyl-3-((3-(8-methyl-9H-purin-6-yl)pyridin-2-yl)amino)phenyl)-4-(trifluoromethyl)picolinamide (3.1 mg, 0.006 mmol, 5%) as a yellow solid. ¹H NMR (500 MHz, DMSO-d₆) δ 13.59 (s, 1H), 12.38 (s, 1H), 10.67 (s, 1H), 9.77 (s, 1H), 8.99 (s, 1H), 8.81 (d, J=4.8 Hz, 1H), 8.71 (s, 1H), 8.34 (s, 1H), 8.10 (d, J=5.3 Hz, 1H), 7.48 (d, J=6.8 Hz, 1H), 7.25 (d, J=8.6 Hz, 1H), 7.04 (dd, J=7.8, 4.4 Hz, 1H), 2.65 (s, 3H), 2.45 (s, 3H). MS (ESI) m/z 523.0 [M+H]⁺

### Example 126. 5-(2-hydroxyethoxy)-N-(4-methyl-3-((3-(8-methyl-9H-purin-6-yl)pyridin-2-yl)amino)phenyl)-4-(trifluoromethyl)picolinamide (Compound IA-129)

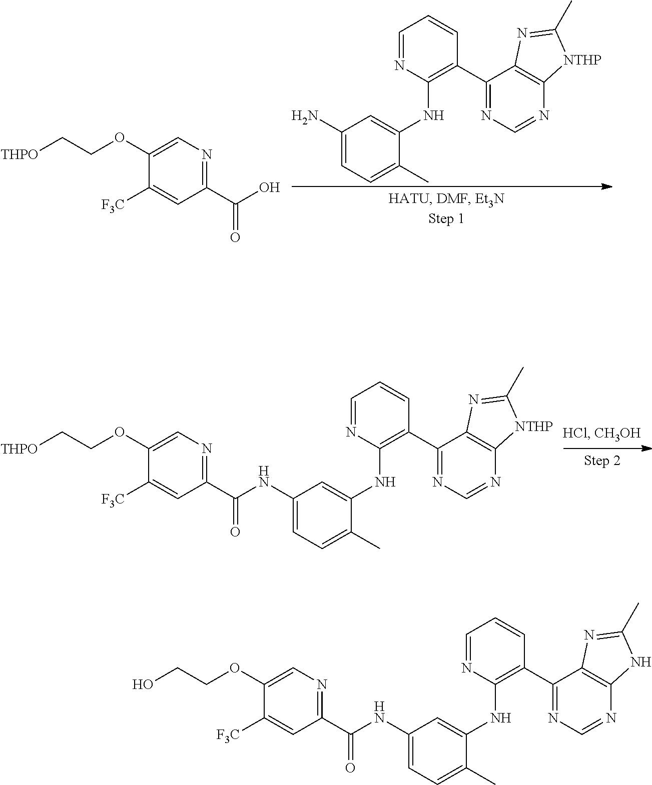

Step 1. A solution of 5-(2-((tetrahydro-2H-pyran-2-yl)oxy)ethoxy)-4-(trifluoromethyl)-picolinic acid (35 mg, 0.10 mmol), 1-[bis(dimethylamino)methylene]-1H-1,2,3-triazolo[4,5-b]pyridinium 3-oxid hexafluorophosphate (114 mg, 0.30 mmol), triethylamine (0.2 mL) and 6-methyl-N1-(3-(8-methyl-9-(tetrahydro-2H-pyran-2-yl)-9H-purin-6-yl)pyridin-2-yl)benzene-1,3-diamine (43 mg, 0.10 mmol) in N,N-dimethylformamide (3 mL) was stirred at room temperature for 2 hours. The reaction solution was diluted with ethyl acetate (50 mL), washed with water (10 mL×3) and brine (10 mL), dried over sodium sulfate, filtered and concentrated. The residue was purified flash chromatography (petroleum ether/ethyl acetate=1/3) to afford N-(4-methyl-3-((3-(8-methyl-9-(tetrahydro-2H-pyran-2-yl)-9H-purin-6-yl)pyridin-2-yl)amino)phenyl)-5-(2-((tetrahydro-2H-pyran-2-yl)oxy)ethoxy)-4-(trifluoromethyl)picolinamide (50 mg, 0.07 mmol, 68.3%) as a brown oil. MS (ESI) m/z 732.9 [M+H]⁺

Step 2. A solution of N-(4-methyl-3-((3-(8-methyl-9-(tetrahydro-2H-pyran-2-yl)-9H-purin-6-yl)pyridin-2-yl)amino)phenyl)-5-(2-((tetrahydro-2H-pyran-2-yl)oxy)ethoxy)-4-(trifluoromethyl)picolinamide (50 mg, 0.07 mmol) in hydrochloric acid (3 M in methanol, 5 mL) was stirred at room temperature for 1 hour. The reaction mixture was concentrated and the pH was adjusted to alkaline with ammonia (7 M in methanol). The resulting mixture was purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford 5-(2-hydroxyethoxy)-N-(4-methyl-3-((3-(8-methyl-9H-purin-6-yl)pyridin-2-yl)amino)phenyl)-4-(trifluoromethyl)picolinamide (10.9 mg, 0.02 mmol, 27.6%). ¹H NMR (500 MHz, DMSO-d₆) δ 13.58 (s, 1H), 12.33 (s, 1H), 10.39 (s, 1H), 9.77 (d, J=7.8 Hz, 1H), 8.98 (s, 1H), 8.80 (d, J=15.6 Hz, 2H), 8.35 (d, J=2.8 Hz, 1H), 8.23 (s, 1H), 7.51 (d, J=8.1 Hz, 1H), 7.24 (d, J=8.3 Hz, 1H), 7.03 (dd, J=7.8, 4.7 Hz, 1H), 5.00 (t, J=5.2 Hz, 1H), 4.47 (t, J=4.8 Hz, 2H), 3.80 (dt, J=13.3, 6.7 Hz, 2H), 2.65 (s, 3H), 2.43 (s, 3H). MS (ESI) m/z 565.1 [M+H]⁺

### Example 127. N-(4-methyl-3-((3-(8-methyl-9H-purin-6-yl)pyridin-2-yl)amino)phenyl)-4-(2,2,2-trifluoro-1-hydroxyethyl)picolinamide (Compound IA-130)

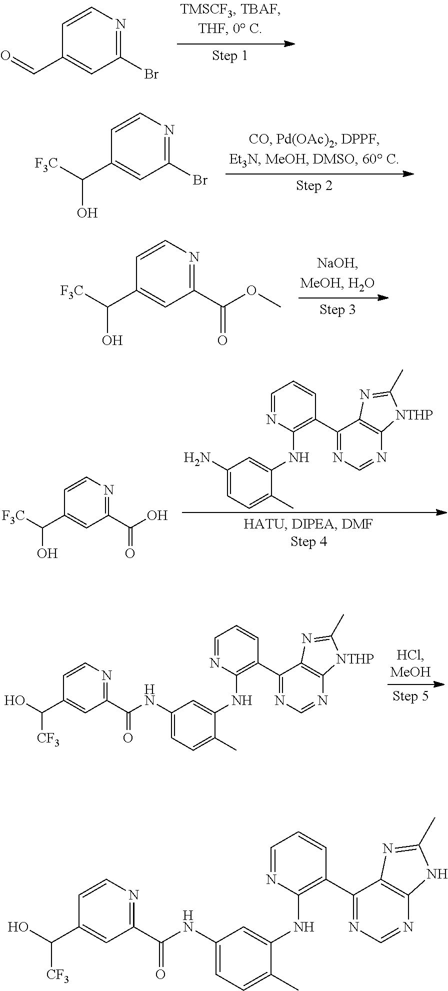

Step 1. To a solution of 2-bromoisonicotinaldehyde (500 mg, 2.68 mmol) in tetrahydrofuran (10 mL) were added trimethyl(trifluoromethyl)silane (460 mg, 3.2 mmol) and tetrabutylammonium fluoride (35.3 mg, 0.135 mmol) at 0° C. The reaction mixture was stirred for 1 hour at room temperature, concentrated, and purified by flash chromatography (ethyl acetate/petroleum ether=4/1) to afford 1-(2-bromopyridin-4-yl)-2,2,2-trifluoroethan-1-ol (641 mg, 2.5 mmol, 93.2%) as a yellow oil. MS (ESI) m/z 256.1 [M+H]⁺

Step 2. To a solution of 1-(2-bromopyridin-4-yl)-2,2,2-trifluoroethan-1-ol (641 mg, 2.50 mmol) in dimethyl sulfoxide (6 mL) and methanol (4 mL) were added triethylamine (1.1 g, 10.0 mmol), 1,1′-bis(diphenylphosphino)ferrocene (692.5 mg, 1.25 mmol) and palladium acetate (112.3 mg, 0.5 mmol) at room temperature. The resulting mixture was stirred for 16 hours at 60° C. under carbon monoxide atmosphere. The reaction mixture was quenched with water/ice (100 mL) and extracted with ethyl acetate (3×40 mL). The combined organic layers were washed with brine (2×100 mL), dried over anhydrous sodium sulfate, filtered and concentrated under reduced pressure. The residue was purified by flash chromatography (ethyl acetate/petroleum ether=1/1) to afford methyl 4-(2,2,2-trifluoro-1-hydroxyethyl)picolinate (358 mg, 1.52 mmol, 61%) as a yellow oil. MS (ESI) m/z 236.1 [M+H]⁺

Step 3. A solution of methyl 4-(2,2,2-trifluoro-1-hydroxyethyl)picolinate (358 mg, 1.52 mmol) and sodium hydroxide (121.6 mg, 3.04 mmol) in methanol (4 mL) and water (2 mL) was stirred for 2 hours at room temperature. The mixture was concentrated in vacuo to afford 4-(2,2,2-trifluoro-1-hydroxyethyl)picolinic acid (218.3 mg, 0.99 mmol, 65%) as a yellow solid. MS (ESI) m/z 222.1 [M+H]⁺

Step 4. To a solution of 4-(2,2,2-trifluoro-1-hydroxyethyl)picolinic acid (100 mg, 0.45 mmol) in N,N-dimethylformamide (2 mL) were added 6-methyl-N1-(3-(8-methyl-9-(tetrahydro-2H-pyran-2-yl)-9H-purin-6-yl)pyridin-2-yl)benzene-1,3-diamine (150.2 mg, 0.36 mmol), 1-[bis(dimethylamino)methylene]-1H-1,2,3-triazolo[4,5-b]pyridinium 3-oxid hexafluorophosphate (256.5 mg, 0.68 mmol) and N,N-diisopropyl ethylamine (174.2 mg, 1.35 mmol) at room temperature. The reaction mixture was stirred for 3 hours, quenched with water (40 mL), and extracted with ethyl acetate (3×40 mL). The combined organic layers were washed with brine (2×50 mL), dried over anhydrous sodium sulfate, filtered and concentrated. The residue was purified by flash chromatography (ethyl acetate/petroleum ether=2/1) to afford N-(4-methyl-3-((3-(8-methyl-9-(tetrahydro-2H-pyran-2-yl)-9H-purin-6-yl)pyridin-2-yl)amino)phenyl)-4-(2,2,2-trifluoro-1-hydroxyethyl)picolinamide (50.8 mg, 0.08 mmol, 18.3%) as a yellow solid. MS (ESI) m/z 619.0 [M+H]⁺

Step 5. A solution of N-(4-methyl-3-((3-(8-methyl-9-(tetrahydro-2H-pyran-2-yl)-9H-purin-6-yl)pyridin-2-yl)amino)phenyl)-4-(2,2,2-trifluoro-1-hydroxyethyl)picolinamide (50.8 mg, 0.08 mmol) in hydrochloric acid (4 M in methanol, 3 mL) was stirred at room temperature for 2 hours. The reaction mixture was concentrated, and the residue was purified by prep-HPLC (Column: SunFire 4.6×50 mm C18, 3.5 μm; Mobile phase: acetonitrile/0.01% aqueous ammonium bicarbonate, 5%-95% over 1.5 min at 2 mL/min) to afford N-(4-methyl-3-((3-(8-methyl-9H-purin-6-yl)pyridin-2-yl)amino)phenyl)-4-(2,2,2-trifluoro-1-hydroxyethyl)picolinamide (4 mg, 0.007 mmol, 9.4%) as a yellow solid. ¹H NMR (400 MHZ, DMSO-d₆) δ 13.58 (s, 1H), 12.34 (s, 1H), 10.49 (s, 1H), 9.78 (d, J=7.8 Hz, 1H), 8.99 (s, 1H), 8.80 (d, J=4.7 Hz, 2H), 8.49-8.17 (m, 2H), 7.79 (d, J=3.9 Hz, 1H), 7.53 (d, J=7.1 Hz, 1H), 7.26 (dd, J=13.8, 7.1 Hz, 2H), 7.04 (dd, J=7.7, 4.7 Hz, 1H), 5.51 (d, J=5.6 Hz, 1H), 2.65 (s, 3H), 2.44 (s, 3H). MS (ESI) m/z 535.0 [M+H]⁺

### Example 128. Synthesis of N-(3-(3-(9H-purin-6-yl)pyridin-2-ylamino)-4-methylphenyl)-2-(azepan-4-yl)acetamide (Compound IA-131)

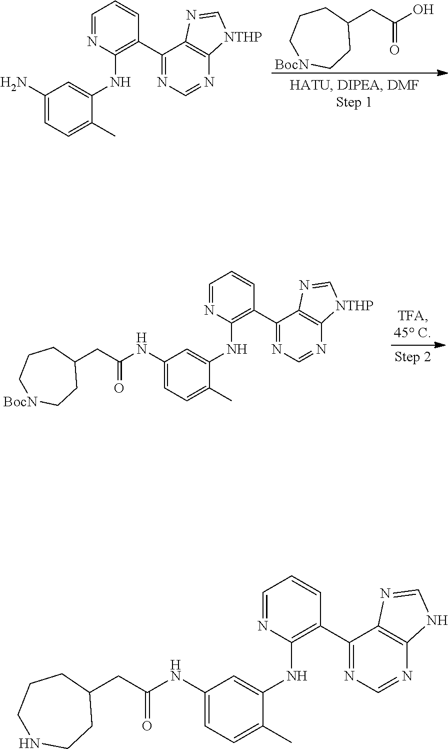

Step 1. A solution of 6-methyl-N1-(3-(9-(tetrahydro-2H-pyran-2-yl)-9H-purin-6-yl)pyridin-2-yl)benzene-1,3-diamine (100 mg, 0.25 mmol), 2-(1-(tert-butoxycarbonyl)azepan-4-yl)acetic acid (77 mg, 0.3 mmol), 1-[bis(dimethylamino)methylene]-1H-1,2,3-triazolo[4,5-b]pyridinium 3-oxid hexafluorophosphate (171 mg, 0.45 mmol) and N,N-diisopropylethylamine (97 mg, 0.75 mmol) in N,N-dimethylformamide (3 mL) was stirred at 25° C. for 1 hour. The mixture was diluted with ethyl acetate (50 mL) and washed with water (50 mL). The organic phase was washed with brine (40 mL×3), dried over sodium sulfate, filtered and concentrated. The residue was purified by flash chromatography (ethyl acetate/petroleum ether=65/100) to afford tert-butyl 4-(2-(4-methyl-3-(3-(9-(tetrahydro-2H-pyran-2-yl)-9H-purin-6-yl)pyridin-2-ylamino)phenylamino)-2-oxoethyl)azepane-1-carboxylate (125 mg, 0.20 mmol, 80%) as a yellow solid. MS (ESI) m/z 641.0 [M+H]⁺

Step 2. A solution of tert-butyl 4-(2-(4-methyl-3-(3-(9-(tetrahydro-2H-pyran-2-yl)-9H-purin-6-yl)pyridin-2-ylamino) phenylamino)-2-oxoethyl)azepane-1-carboxylate (125 mg, 0.20 mmol) in trifluoroacetic acid (5 mL) was stirred at 45° C. for 2 hours. The reaction mixture was concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) afford N-(3-(3-(9H-purin-6-yl)pyridin-2-ylamino)-4-methylphenyl)-2-(azepan-4-yl)acetamide (66.2 mg, 0.15 mmol, 75%) as a yellow solid. ¹H NMR (400 MHZ, DMSO-d₆) δ 12.66-12.51 (m, 1H), 9.96-9.86 (m, 1H), 9.84-9.75 (m, 1H), 8.95-8.86 (m, 1H), 8.54-8.40 (m, 2H), 8.32-8.22 (m, 1H), 7.39 (dd, J=8.2, 1.9 Hz, 1H), 7.13 (d, J=8.3 Hz, 1H), 6.99 (dd, J=7.8, 4.7 Hz, 1H), 3.52-2.87 (m, 4H), 2.41 (s, 3H), 2.30-2.19 (m, 2H), 2.15-2.03 (m, 1H), 1.99-1.08 (m, 6H). MS (ESI) m/z 457.0 [M+H]⁺

### Example 129. Synthesis of N-(3-(3-(9H-purin-6-yl)pyridin-2-ylamino)-4-methylphenyl)-2-(1-methylazepan-4-yl)acetamide (Compound IA-132)

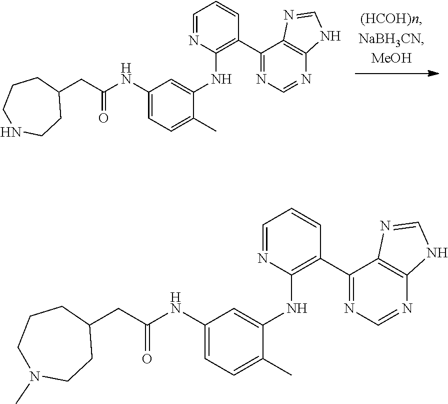 [SMILES: Cc1ccc(NC(=O)CC2CCCNCC2)cc1Nc1ncccc1-c1ncnc2[nH]cnc12 ; Cc1ccc(NC(=O)CC2CCCN(C)CC2)cc1Nc1ncccc1-c1ncnc2[nH]cnc12]

A mixture of N-(3-(3-(9H-purin-6-yl)pyridin-2-ylamino)-4-methylphenyl)-2-(azepan-4-yl)acetamide (30 mg, 0.065 mmol) and paraformaldehyde (10 mg, 0.325 mmol) in methanol (5 mL) was stirred at 25° C. for 1 hour under nitrogen. Then sodium cyanoborohydride (20 mg, 0.325 mmol) was added, and the reaction mixture was stirred at 25° C. for 2 hours. The reaction mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-(3-(9H-purin-6-yl)pyridin-2-ylamino)-4-methylphenyl)-2-(1-methylazepan-4-yl)acetamide (14.8 mg, 0.031 mmol, 48%) as a yellow solid. ¹H NMR (400 MHZ, DMSO-d₆) δ 12.30 (s, 1H), 9.86-9.70 (m, 2H), 9.03 (s, 1H), 8.66 (s, 1H), 8.43 (d, J=1.9 Hz, 1H), 8.33 (dd, J=4.6, 1.8 Hz, 1H), 7.39 (dd, J=8.1, 1.9 Hz, 1H), 7.14 (d, J=8.3 Hz, 1H), 7.02 (dd, J=7.9, 4.7 Hz, 1H), 2.81-2.54 (m, 4H), 2.39 (s, 3H), 2.35 (s, 3H), 2.24 (d, J=7.2 Hz, 2H), 2.15-2.05 (m, 1H), 1.82-1.67 (m, 3H), 1.63-1.52 (m, 1H), 1.52-1.40 (m, 1H), 1.38-1.25 (m, 1H). MS (ESI) m/z 471.0 [M+H]⁺

### Example 130. Synthesis of N-(3-((3-(9H-purin-6-yl)pyridin-2-yl)amino)-4-methylphenyl)-2-(1,4-oxazepan-4-yl)acetamide (Compound IA-133)

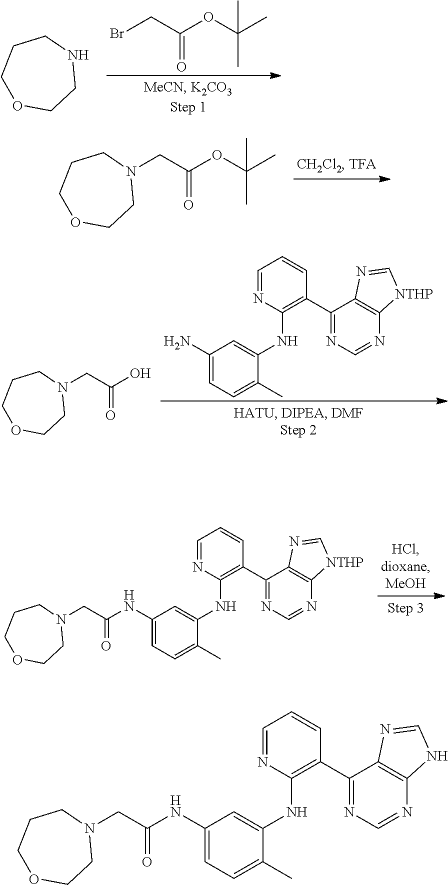

Step 1. To a mixture of 1,4-oxazepane (237 mg, 2.35 mmol) and potassium carbonate (1619 mg, 11.7 mmol) in acetonitrile (6 mL) was added tert-butyl 2-bromoacetate (455 mg, 2.35 mmol). The reaction mixture was stirred at room temperature for 16 hours. The mixture was diluted with ethyl acetate and filtered. The filtrate was concentrated under reduced pressure to give crude tert-butyl 2-(1,4-oxazepan-4-yl)acetate (200 mg, 0.93 mmol) as a yellow oil. MS (ESI) m/z 216.3 [M+H]⁺

Step 2. To a mixture of tert-butyl 2-(1,4-oxazepan-4-yl)acetate (200 mg, 0.93 mmol) in dichloromethane (3 mL) added trifluoroacetic acid (3 mL). The mixture was stirred at room temperature for 16 hours and concentrated to give crude 2-(1,4-oxazepan-4-yl)acetic acid (100 mg, 0.63 mmol) as a yellow oil, which was directly used to the next step. MS (ESI) m/z 160.1 [M+H]⁺

Step 3. To a solution of 6-methyl-N1-(3-(9-(tetrahydro-2H-pyran-2-yl)-9H-purin-6-yl)pyridin-2-yl)benzene-1,3-diamine (126 mg, 0.31 mmol) in N,N-dimethylformamide (3 mL) was added N,N-diisopropylethylamine (122 mg, 0.94 mmol), 2-(7-azabenzotriazol-1-yl)-N,N,N′,N′-tetramethyluronium hexafluorophosphate (179 mg, 0.47 mmol) and 2-(1,4-oxazepan-4-yl)acetic acid (50 mg, 0.31 mmol). The mixture was stirred at room temperature for 1 hour. The mixture was extracted with ethyl acetate (30 mL×3). The combined organic layers were washed with brine (30 mL), dried over anhydrous sodium sulfate, and filtered. The filtrate was concentrated under reduced pressure. The residue was purified by flash chromatography (ethyl acetate) to give N-(4-methyl-3-((3-(9-(tetrahydro-2H-pyran-2-yl)-9H-purin-6-yl)pyridin-2-yl)amino)phenyl)-2-(1,4-oxazepan-4-yl)acetamide (100 mg, 0.18 mmol, 59%) as a yellow solid. MS (ESI) m/z 543.0 [M+H]⁺

Step 4. To a solution of N-(4-methyl-3-((3-(9-(tetrahydro-2H-pyran-2-yl)-9H-purin-6-yl)pyridin-2-yl)amino)phenyl)-2-(1,4-oxazepan-4-yl)acetamide (100 mg, 0.18 mmol) in methanol (4 mL) was added hydrochloric acid (4 M in dioxane, 4 mL) at 25° C., and the mixture was stirred at 25° C. for 1 hour. The reaction mixture was concentrated and methanol (10 mL) was added. The pH was adjusted to 8 with ammonia (7 N in methanol). The mixture was concentrated under reduced pressure and diluted with methanol (8 mL). The solid was collected to give N-(3-((3-(9H-purin-6-yl)pyridin-2-yl)amino)-4-methylphenyl)-2-(1,4-oxazepan-4-yl)acetamide (90.0 mg, 0.20 mmol, 85%) as a yellow solid. ¹H NMR (400 MHZ, DMSO-d₆) δ 1.91 (t, J=5.2 Hz, 2H), 2.40 (s, 3H), 2.91 (s, 4H), 3.34 (s, 2H), 3.73 (t, J=6.0 Hz, 4H), 7.03 (q, J=4.8 Hz, 1H), 7.18 (d, J=8.0 Hz, 1H), 7.36 (dd, J_{1=1.2} Hz, J₂=8.0 Hz, 1H), 8.33 (q, J=1.6 Hz, 1H), 8.52 (s, 1H), 8.72 (s, 1H), 9.07 (s, 1H), 9.81 (s, 2H), 12.30 (s, 1H), 13.84 (s, 1H). MS (ESI) m/z 459.0[M+H]⁺

### Example 131. Synthesis of N-(3-((3-(9H-purin-6-yl)pyridin-2-yl)amino)-4-methylphenyl)-2-(1,4-diazepan-1-yl)acetamide (Compound IA-134)

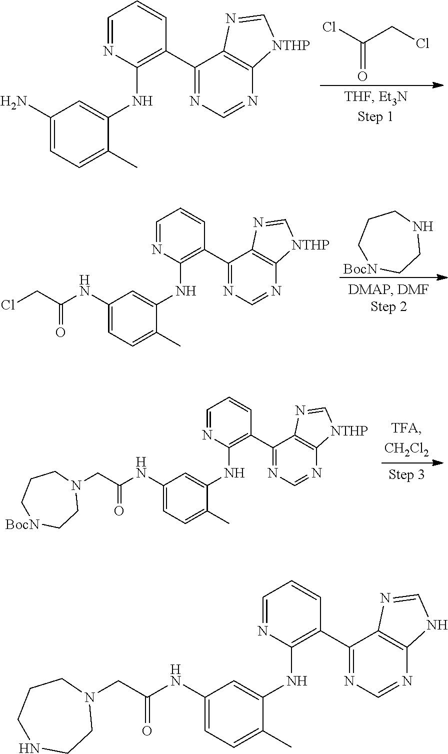

Step 1. To a solution of 6-methyl-N1-(3-(9-(tetrahydro-2H-pyran-2-yl)-9H-purin-6-yl)pyridin-2-yl)benzene-1,3-diamine (300 mg, 0.75 mmol) in tetrahydrofuran (10 mL) was added triethylamine (758 mg, 7.5 mmol) and 2-chloroacetyl chloride (170 mg, 1.5 mmol). The mixture was stirred at room temperature for 5 minutes. The reaction mixture was concentrated and purified by flash chromatography (petroleum ether/ethyl acetate=1/5) to give 2-chloro-N-(4-methyl-3-((3-(9-(tetrahydro-2H-pyran-2-yl)-9H-purin-6-yl)pyridin-2-yl)amino)phenyl)acetamide (273 mg, 0.57 mmol, 76.3%) as a yellow solid. MS (ESI) m/z 478.1 [M+H]⁺

Step 2. To a solution of 2-chloro-N-(4-methyl-3-((3-(9-(tetrahydro-2H-pyran-2-yl)-9H-purin-6-yl)pyridin-2-yl) amino)phenyl)acetamide (90 mg, 0.19 mmol) in N,N-dimethylformamide (6 mL) was added tert-butyl 1,4-diazepane-1-carboxylate (57 mg, 0.29 mmol) and 4-dimethylaminopyridine (59 mg, 0.48 mmol). The mixture was stirred at room temperature for 16 hours. The mixture was washed with brine and extracted with ethyl acetate (30 mL×3). The combined organic phases were dried over sodium sulfate, filtered and concentrated under reduced pressure to give tert-butyl 4-(2-((4-methyl-3-((3-(9-(tetrahydro-2H-pyran-2-yl)-9H-purin-6-yl)pyridin-2-yl)amino)phenyl)amino)-2-oxoethyl)-1,4-diazepane-1-carboxylate (76 mg, 0.12 mmol, 62.4%) as a yellow oil. MS (ESI) m/z 642.3 [M+H]⁺

Step 3. To a solution of tert-butyl 4-(2-((4-methyl-3-((3-(9-(tetrahydro-2H-pyran-2-yl)-9H-purin-6-yl)pyridin-2-yl)amino)phenyl)amino)-2-oxoethyl)-1,4-diazepane-1-carboxylate (76 mg, 0.12 mmol) in dichloromethane (10 mL) was added trifluoroacetic acid (4 mL). The mixture stirred at room temperature for 2 hours. The resulting mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-2-(1,4-diazepan-1-yl)acetamide as a yellow solid (4.9 mg, 0.01 mmol, 9%). ¹H NMR (400 MHz, CD₃OD) δ 9.57 (d, J=7.8 Hz, 1H), 8.99 (s, 1H), 8.50 (s, 2H), 8.35 (d, J=2.0 Hz, 1H), 8.24 (dd, J=4.8, 1.8 Hz, 1H), 7.32 (dd, J=8.2, 2.1 Hz, 1H), 7.20 (d, J=8.2 Hz, 1H), 6.96 (dd, J=7.8, 4.8 Hz, 1H), 3.46 (s, 2H), 3.33 (dd, J=8.2, 6.3 Hz, 4H), 3.10-3.03 (m, 2H), 2.90 (t, J=5.8 Hz, 2H), 2.42 (s, 3H), 2.04 (dt, J=11.3, 5.7 Hz, 2H). MS (ESI) m/z 457.8 [M+H]⁺

### Example 132. Synthesis of N-(3-((3-(9H-purin-6-yl)pyridin-2-yl)amino)-4-methylphenyl)-2-(4-methyl-1,4-diazepan-1-yl)acetamide (Compound IA-135)

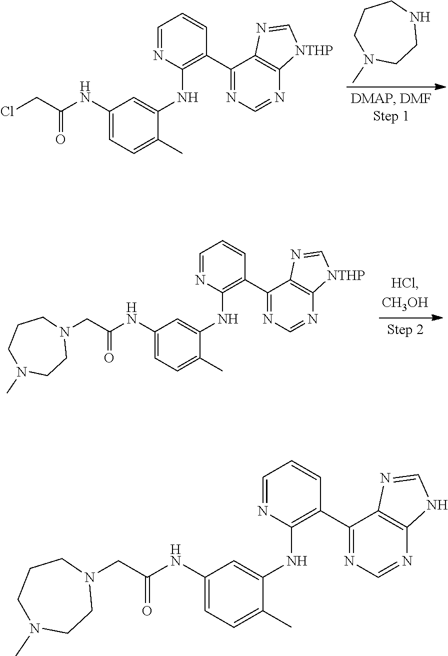

Step 1. To a solution of 2-chloro-N-(4-methyl-3-((3-(9-(tetrahydro-2H-pyran-2-yl)-9H-purin-6-yl)pyridin-2-yl) amino)phenyl)acetamide (90 mg, 0.19 mmol) in N,N-dimethylformamide (6 mL) was added 1-methyl-1,4-diazepane (33 mg, 0.29 mmol) and 4-dimethylaminopyridine (70 mg, 0.57 mmol). The mixture was stirred at room temperature for 4 hours. The mixture was washed with brine and extracted with ethyl acetate (30 mL×3). The combined organic phases were dried over sodium sulfate, filtered and concentrated under reduced pressure to give 2-(4-methyl-1,4-diazepan-1-yl)-N-(4-methyl-3-((3-(9-(tetrahydro-2H-pyran-2-yl)-9H-purin-6-yl)pyridin-2-yl)amino)phenyl)acetamide (79 mg, 0.14 mmol, 75%) as a yellow oil.

Step 2. A mixture of 2-(4-methyl-1,4-diazepan-1-yl)-N-(4-methyl-3-((3-(9-(tetrahydro-2H-pyran-2-yl)-9H-purin-6-yl)pyridin-2-yl)amino)phenyl)acetamide (79 mg, 0.14 mmol) in hydrochloric acid (3 M in methanol, 5 mL) was stirred at room temperature for 2 hours. The reaction mixture was concentrated, and then the pH was adjusted to alkaline with ammonia (7 M in methanol). The resulting mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(3-((3-(9H-purin-6-yl)pyridin-2-yl)amino)-4-methylphenyl)-2-(4-methyl-1,4-diazepan-1-yl)acetamide as a yellow solid (6.2 mg, 0.01 mmol, 9.4%). ¹H NMR (400 MHZ, CD₃OD) δ 9.95 (s, 1H), 9.00 (d, J=5.7 Hz, 1H), 8.56 (s, 1H), 8.13 (dd, J=17.2, 2.9 Hz, 2H), 7.48 (dd, J=8.2, 1.9 Hz, 1H), 7.33 (d, J=8.3 Hz, 1H), 7.11 (dd, J=7.7, 5.5 Hz, 1H), 3.60 (s, 2H), 3.49 (s, 4H), 3.24 (d, J=9.2 Hz, 2H), 3.03 (s, 2H), 2.95 (s, 3H), 2.38 (s, 3H), 2.19-2.10 (m, 2H). MS (ESI) m/z 471.8 [M+H]⁺

### Example 133. Synthesis of N-(3-(3-(9H-purin-6-yl)pyridin-2-ylamino)-4-methylphenyl)-2-(4-methyl-1,4-oxazepan-7-yl)acetamide (Compound IA-136)

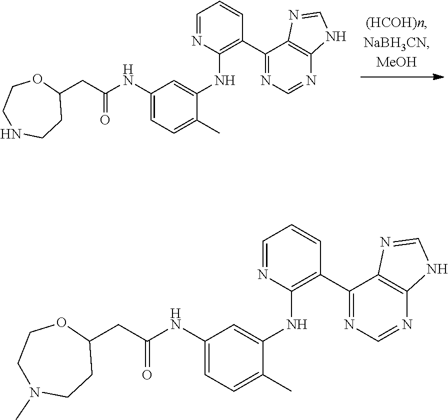 [SMILES: Cc1ccc(NC(=O)CC2CCNCCO2)cc1Nc1ncccc1-c1ncnc2[nH]cnc12 ; Cc1ccc(NC(=O)CC2CCN(C)CCO2)cc1Nc1ncccc1-c1ncnc2[nH]cnc12]

A solution of N-(3-(3-(9H-purin-6-yl)pyridin-2-ylamino)-4-methylphenyl)-2-(1,4-oxazepan-7-yl)acetamide (0.020 g, 0.04 mmol), paraformaldehyde (0.007 g, 0.22 mmol) and sodium cyanoborohydride (0.014 g, 0.22 mmol) in methanol (2 mL) was stirred at room temperature for 6 hours. The mixture was diluted with water (20 mL), and extracted with dichloromethane (50 mL). The organic layer was washed with water (20 mL), dried over anhydrous sodium sulfate, filtered and concentrated. The residue was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-(3-(9H-purin-6-yl)pyridin-2-ylamino)-4-methylphenyl)-2-(4-methyl-1,4-oxazepan-7-yl)acetamide (3.8 mg, 0.007 mmol, 18.4%) as a yellow solid. ¹H NMR (400 MHZ, CD₃OD): δ 9.62 (brs, 1H), 9.03 (s, 1H), 8.54 (s, 1H), 8.34 (s, 1H), 8.27 (d, J=4.4 Hz, 1H), 7.33 (d, J=6.0 Hz, 1H), 7.21 (d, J=8.8 Hz, 1H), 7.00 (d, J=7.6 Hz, 1H), 4.34-4.28 (m, 1H), 4.07-4.01 (m, 1H), 3.87-3.81 (m, 1H), 3.50-3.39 (m, 4H), 2.93 (s, 1H), 2.64-2.61 (m, 1H), 2.56-2.53 (m, 1H), 2.48 (s, 1H), 2.31-2.12 (m, 2H). MS (ESI) m/z 473.3 [M+H]⁺

### Examples 134-137. Synthesis of N-(3-(3-(9H-purin-6-yl)pyridin-2-ylamino)-4-methylphenyl)-2-((1S,3R)-3-(trifluoromethyl)cyclohexyl)acetamide, N-(3-(3-(9H-purin-6-yl)pyridin-2-ylamino)-4-methylphenyl)-2-((1R,3S)-3-(trifluoromethyl)cyclohexyl)acetamide, N-(3-(3-(9H-purin-6-yl)pyridin-2-ylamino)-4-methylphenyl)-2-((1S,3S)-3-(trifluoromethyl)cyclohexyl)acetamide, and N-(3-(3-(9H-purin-6-yl)pyridin-2-ylamino)-4-methylphenyl)-2-((1R,3R)-3-(trifluoromethyl)cyclohexyl)acetamide (Compound IA-137, Compound IA-138, Compound IA-139, Compound IA-140)

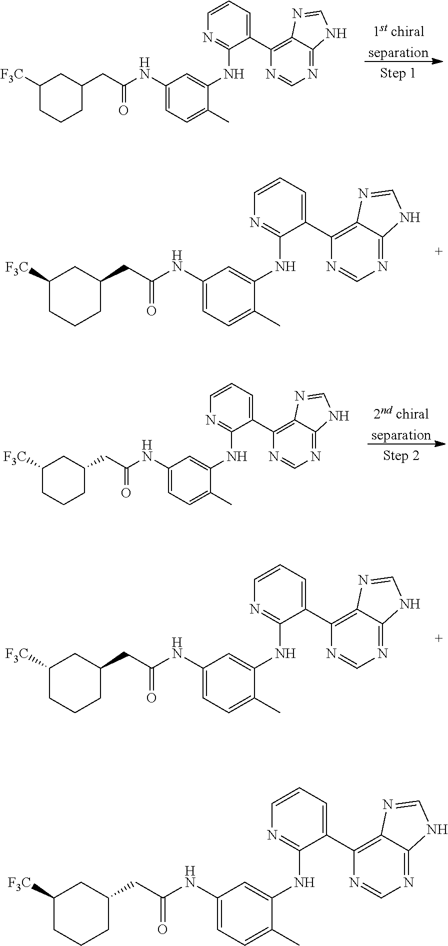 [SMILES: Cc1ccc(NC(=O)CC2CCCC(C(F)(F)F)C2)cc1Nc1ncccc1-c1ncnc2[nH]cnc12 ; Cc1ccc(NC(=O)C[C@H]2CCC[C@@H](C(F)(F)F)C2)cc1Nc1ncccc1-c1ncnc2[nH]cnc12 ; Cc1ccc(NC(=O)C[C@@H]2CCC[C@H](C(F)(F)F)C2)cc1Nc1ncccc1-c1ncnc2[nH]cnc12 ; Cc1ccc(NC(=O)C[C@H]2CCC[C@H](C(F)(F)F)C2)cc1Nc1ncccc1-c1ncnc2[nH]cnc12 ; Cc1ccc(NC(=O)C[C@@H]2CCC[C@@H](C(F)(F)F)C2)cc1Nc1ncccc1-c1ncnc2[nH]cnc12]

Step 1. A diastereomeric and racemic mixture of N-(3-(3-(9H-purin-6-yl)pyridin-2-ylamino)-4-methylphenyl)-2-(3-(trifluoromethyl)cyclohexyl)acetamide (100 mg, 0.219 mmol) was separated by chiral prep-HPLC (Instrument: SFC; Column: AS-H, 4.6×100 mm, 5 μm; Mobile phase: CO₂/MeOH (0.2% methanol ammonia)) to give separated isomers, arbitrarily assigned as N-(3-(3-(9H-purin-6-yl)pyridin-2-ylamino)-4-methylphenyl)-2-((1S,3R)-3-(trifluoromethyl)cyclohexyl)acetamide (5.3 mg) as a yellow solid, ¹H NMR (400 MHZ, DMSO-d₆) δ 12.28 (s, 1H), 9.87-9.73 (m, 2H), 9.05 (s, 1H), 8.68 (s, 1H), 8.43 (d, J=2.0 Hz, 1H), 8.33 (dd, J=4.7, 1.8 Hz, 1H), 7.38 (d, J=8.1 Hz, 1H), 7.15 (d, J=8.2 Hz, 1H), 7.02 (dd, J=7.8, 4.7 Hz, 1H), 2.45-2.27 (m, 6H), 1.83-1.74 (m, 1H), 1.69-1.62 (m, 1H), 1.59-1.16 (m, 7H). MS (ESI) m/z 510.1 [M+H]⁺; and N-(3-(3-(9H-purin-6-yl)pyridin-2-ylamino)-4-methylphenyl)-2-((1R,3S)-3-(trifluoromethyl)cyclohexyl)acetamide (6.2 mg) as a yellow solid, ¹H NMR (400 MHZ, DMSO-d₆) δ 12.30 (s, 1H), 9.85-9.76 (m, 2H), 9.04 (s, 1H), 8.67 (s, 1H), 8.43 (s, 1H), 8.33 (dd, J=4.7, 2.0 Hz, 1H), 7.38 (d, J=10.1 Hz, 1H), 7.15 (d, J=8.2 Hz, 1H), 7.02 (dd, J=7.9, 4.7 Hz, 1H), 2.44-2.27 (m, 6H), 1.84-1.74 (m, 1H), 1.67-1.61 (m, 1H), 1.61-1.21 (m, 7H). MS (ESI) m/z 510.1 [M+H]⁺; and the two remaining isomers as a mixture (50 mg).

Step 2. The above mixture (50 mg) was further separated by chiral-HPLC (Instrument: Gilson-281; Column: AD 20×250 mm, 10 μm; Mobile Phase: n-Hexane (0.1% diethylamine)/EtOH (0.1% diethylamine)=80/20) to give separated isomers, arbitrarily assigned as N-(3-(3-(9H-purin-6-yl)pyridin-2-ylamino)-4-methylphenyl)-2-((1S,3S)-3-(trifluoromethyl)cyclohexyl)acetamide (4.1 mg) as a yellow solid, ¹H NMR (400 MHZ, DMSO-d₆) δ 12.28 (s, 1H), 9.88-9.73 (m, 2H), 9.05 (s, 1H), 8.68 (s, 1H), 8.44 (s, 1H), 8.33 (d, J=2.8 Hz, 1H), 7.38 (d, J=10.1 Hz, 1H), 7.15 (d, J=8.2 Hz, 1H), 7.03 (dd, J=7.8, 4.7 Hz, 1H), 2.39 (s, 3H), 2.35-2.29 (m, 1H), 2.28-2.22 (m, 2H), 1.95-1.67 (m, 5H), 1.40-1.28 (m, 1H), 1.20-1.08 (m, 1H), 1.02-0.87 (m, 2H). MS (ESI) m/z 510.1 [M+H]⁺ *; and N-(3-(3-(9H-purin-6-yl)pyridin-2-ylamino)-4-methylphenyl)-2-((1R,3R)-3-(trifluoromethyl)cyclohexyl)acetamide (5.3 mg) as a yellow solid, ¹H NMR (400 MHz, DMSO-d₆) δ 12.46 (s, 1H), 9.92-9.76 (m, 2H), 8.96 (s, 1H), 8.55 (s, 1H), 8.45 (s, 1H), 8.30 (d, J=4.6 Hz, 1H), 7.38 (d, J=6.2 Hz, 1H), 7.14 (d, J=7.8 Hz, 1H), 7.01 (dd, J=7.8, 4.7 Hz, 1H), 2.40 (s, 3H), 2.36-2.30 (m, 1H), 2.29-2.21 (m, 2H), 1.96-1.68 (m, 5H), 1.39-1.28 (m, 1H), 1.20-1.10 (m, 1H), 1.06-0.89 (m, 2H). MS (ESI) m/z 510.1 [M+H]⁺

### Examples 138-141. Synthesis of N-(3-(3-(9H-purin-6-yl)pyridin-2-ylamino)-4-methylphenyl)-2-((2R,4S)-2-(trifluoromethyl)piperidin-4-yl)acetamide, N-(3-(3-(9H-purin-6-yl)pyridin-2-ylamino)-4-methylphenyl)-2-((2S,4R)-2-(trifluoromethyl)piperidin-4-yl)acetamide, N-(3-(3-(9H-purin-6-yl)pyridin-2-ylamino)-4-methylphenyl)-2-((2S,4S)-2-(trifluoromethyl)piperidin-4-yl)acetamide, and N-(3-(3-(9H-purin-6-yl)pyridin-2-ylamino)-4-methylphenyl)-2-((2R,4R)-2-(trifluoromethyl)piperidin-4-yl)acetamide (Compound IA-141, Compound IA-142, Compound IA-143, Compound IA-144)

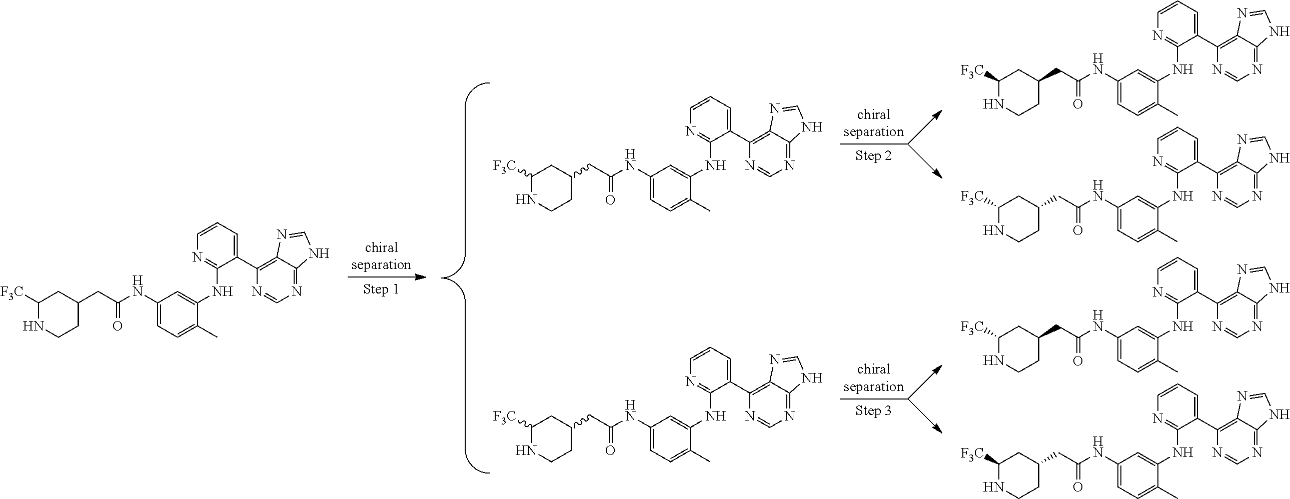

Step 1. A diastereomeric and racemic mixture of 4 isomers of N-(3-(3-(9H-purin-6-yl)pyridin-2-ylamino)-4-methylphenyl)-2-(2-(trifluoromethyl)piperidin-4-yl)acetamide (100 mg, 0.2 mmol) was separated by chiral prep-HPLC (Instrument: SFC-150 (Waters); Column: AS 20×250 mm, 10 μm (Daicel); Mobile phase: CO₂/MeOH (0.2% methanol ammonia)=65/35) to give two mixtures of two isomers each (50 mg each).

Step 2. The first of the above mixtures was further separated by chiral prep-HPLC (Instrument: SFC-80 (Thar, Waters); Column: AD 20×250 mm, 10 μm (Daicel); Mobile phase: CO₂/MeOH (0.2% methanol ammonia)=50/50) to give separated isomers as yellow solids, arbitrarily assigned as N-(3-(3-(9H-purin-6-yl)pyridin-2-ylamino)-4-methylphenyl)-2-((2R,4S)-2-(trifluoromethyl)piperidin-4-yl)acetamide (24.1 mg), ¹H NMR (400 MHZ, DMSO-d₆) δ 13.87 (s, 1H), 12.28 (s, 1H), 9.84-9.81 (m, 2H), 9.07 (s, 1H), 8.72 (s, 1H), 8.44 (s, 1H), 8.34 (d, J=3.0 Hz, 1H), 7.39 (d, J=7.7 Hz, 1H), 7.15 (d, J=8.3 Hz, 1H), 7.03 (dd, J=7.8, 4.8 Hz, 1H), 3.29-3.16 (m, 1H), 3.03-2.94 (m, 1H), 2.61-2.54 (m, 1H), 2.39 (s, 3H), 2.27 (d, J=4.2 Hz, 2H), 2.00-1.86 (m, 1H), 1.83-1.72 (m, 1H), 1.66-1.55 (m, 1H), 1.15-0.94 (m, 2H). MS (ESI) m/z 510.8 [M+H]; and N-(3-(3-(9H-purin-6-yl)pyridin-2-ylamino)-4-methylphenyl)-2-((2S,4R)-2-(trifluoromethyl)piperidin-4-yl)acetamide (23.2 mg), ¹H NMR (400 MHZ, DMSO-d₆) δ 13.86 (s, 1H), 12.28 (s, 1H), 9.90-9.78 (m, 2H), 9.07 (s, 1H), 8.72 (s, 1H), 8.44 (s, 1H), 8.34 (dd, J=4.7, 1.9 Hz, 1H), 7.42-7.35 (m, 1H), 7.15 (d, J=8.4 Hz, 1H), 7.03 (dd, J=7.9, 4.7 Hz, 1H), 3.27-3.15 (m, 1H), 3.02-2.94 (m, 1H), 2.59-2.51 (m, 1H), 2.39 (s, 3H), 2.30-2.24 (m, 2H), 2.00-1.87 (m, 1H), 1.85-1.74 (m, 1H), 1.65-1.57 (m, 1H), 1.14-0.99 (m, 2H). MS (ESI) m/z 510.8 [M+H]⁺

Step 3. The second of the above mixtures was further separated by chiral prep-HPLC (Instrument: SFC-80 (Thar, Waters); Column: AD 20×250 mm, 10 μm (Daicel); Mobile phase: CO₂/MeOH (0.5% methanol ammonia)=45/55) to give separated isomers as yellow solids, arbitrarily assigned as N-(3-(3-(9H-purin-6-yl)pyridin-2-ylamino)-4-methylphenyl)-2-((2S,4S)-2-(trifluoromethyl)piperidin-4-yl)acetamide (21.4 mg), ¹H NMR (400 MHZ, DMSO-d₆) δ 13.84 (s, 1H), 12.26 (s, 1H), 9.96-9.69 (m, 2H), 9.07 (s, 1H), 8.72 (s, 1H), 8.43 (s, 1H), 8.34 (d, J=4.4 Hz, 1H), 7.38 (d, J=8.1 Hz, 1H), 7.15 (d, J=8.2 Hz, 1H), 7.03 (dd, J=7.7, 4.6 Hz, 1H), 3.51-3.39 (m, 1H), 2.85-2.73 (m, 2H), 2.43-2.20 (m, 6H), 1.76-1.67 (m, 1H), 1.65-1.56 (m, 1H), 1.52-1.41 (m, 1H), 1.28-1.16 (m, 1H). MS (ESI) m/z 510.8 [M+H]⁺; and N-(3-(3-(9H-purin-6-yl)pyridin-2-ylamino)-4-methylphenyl)-2-((2R,4R)-2-(trifluoromethyl)piperidin-4-yl)acetamide (24.7 mg), ¹H NMR (400 MHZ, DMSO-d₆) δ 13.83 (s, 1H), 12.26 (s, 1H), 9.99-9.61 (m, 2H), 9.07 (s, 1H), 8.72 (s, 1H), 8.43 (d, J=2.0 Hz, 1H), 8.34 (dd, J=4.7, 1.9 Hz, 1H), 7.38 (dd, J=8.2, 2.0 Hz, 1H), 7.15 (d, J=8.3 Hz, 1H), 7.03 (dd, J=7.9, 4.7 Hz, 1H), 3.50-3.39 (m, 1H), 2.82-2.70 (m, 2H), 2.46-2.19 (m, 6H), 1.74-1.67 (m, 1H), 1.65-1.59 (m, 1H), 1.53-1.44 (m, 1H), 1.28-1.20 (m, 1H). MS (ESI) m/z 511.1 [M+H]⁺

### Example 142. Synthesis of N-(3-((3-(9H-purin-6-yl)pyridin-2-yl)amino)-4-methylphenyl)-3-(trifluoromethyl)benzamide (Compound IA-145)

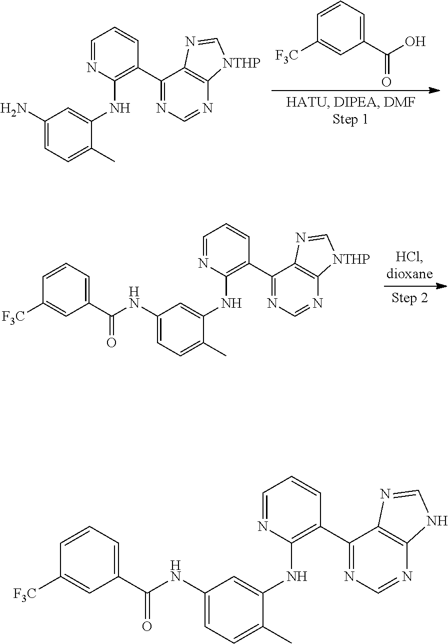

Step 1. A solution of 3-(trifluoromethyl)benzoic acid (19.0 mg, 0.1 mmol), 6-methyl-N1-(3-(9-(tetrahydro-2H-pyran-2-yl)-9H-purin-6-yl)pyridin-2-yl)benzene-1,3-diamine (40.1 mg, 0.1 mmol), 1-[bis(dimethylamino)methylene]-1H-1,2,3-triazolo[4,5-b]pyridinium 3-oxid hexafluorophosphate (45.6 mg, 0.12 mmol), and N,N-diisopropylethylamine (38.7 mg, 0.3 mmol) in N,N-dimethylformamide (0.5 mL) was stirred at 25° C. for 2 hours. The reaction mixture was concentrated. The residue was triturated in petroleum ether/ethyl acetate=1/4 to give N-(4-methyl-3-((3-(9-(tetrahydro-2H-pyran-2-yl)-9H-purin-6-yl)pyridin-2-yl)amino)phenyl)-3-(trifluoromethyl)benzamide (51.9 mg, 0.09 mmol, 90%) as a yellow solid. MS (ESI) m/z 574.2 [M+H]⁺

Step 2. A solution of N-(4-methyl-3-((3-(9-(tetrahydro-2H-pyran-2-yl)-9H-purin-6-yl)pyridin-2-yl)amino)phenyl)-3-(trifluoromethyl)benzamide (57 mg, 0.1 mmol) in hydrochloric acid (4 M in dioxane, 1 mL) was stirred at 25° C. for 4 hours. The reaction mixture was concentrated. The crude product was purified by prep-HPLC (Column: BOSTON pHlex ODS 10 μm, 21.2×250 mm, 120 Å; Mobile phase: acetonitrile/0.1% ammonium bicarbonate) to give N-(3-((3-(9H-purin-6-yl)pyridin-2-yl)amino)-4-methylphenyl)-3-(trifluoromethyl)benzamide (24.6 mg, 0.05 mmol, 50%) as a yellow solid. ¹H NMR (400 MHZ, DMSO-d₆) δ 13.8 (s, 1H), 12.3 (s, 1H), 10.4 (s, 1H), 9.8 (s, 1H), 9.2 (s, 1H), 8.6-8.7 (d, 2H), 7.96 (d, 1H), 7.8 (d, 1H), 7.5 (s, 1H), 7.2 (s, 1H), 7.03 (s, 1H), 2.67 (s, 3H). MS (ESI) m/z 490.2 [M+H]⁺

### Examples 143-146. Synthesis of N-(3-((3-(9H-purin-6-yl)pyridin-2-yl)amino)-4-methylphenyl)-2-((2R,4S)-2-(trifluoromethyl)tetrahydro-2H-pyran-4-yl)acetamide, N-(3-((3-(9H-purin-6-yl)pyridin-2-yl)amino)-4-methylphenyl)-2-((2S,4S)-2-(trifluoromethyl)tetrahydro-2H-pyran-4-yl)acetamide, N-(3-((3-(9H-purin-6-yl)pyridin-2-yl)amino)-4-methylphenyl)-2-((2R,4R)-2-(trifluoromethyl)tetrahydro-2H-pyran-4-yl)acetamide, and N-(3-((3-(9H-purin-6-yl)pyridin-2-yl)amino)-4-methylphenyl)-2-((2S,4R)-2-(trifluoromethyl)tetrahydro-2H-pyran-4-yl)acetamide (Compound IA-146, Compound IA-147, Compound IA-148, Compound IA-149)

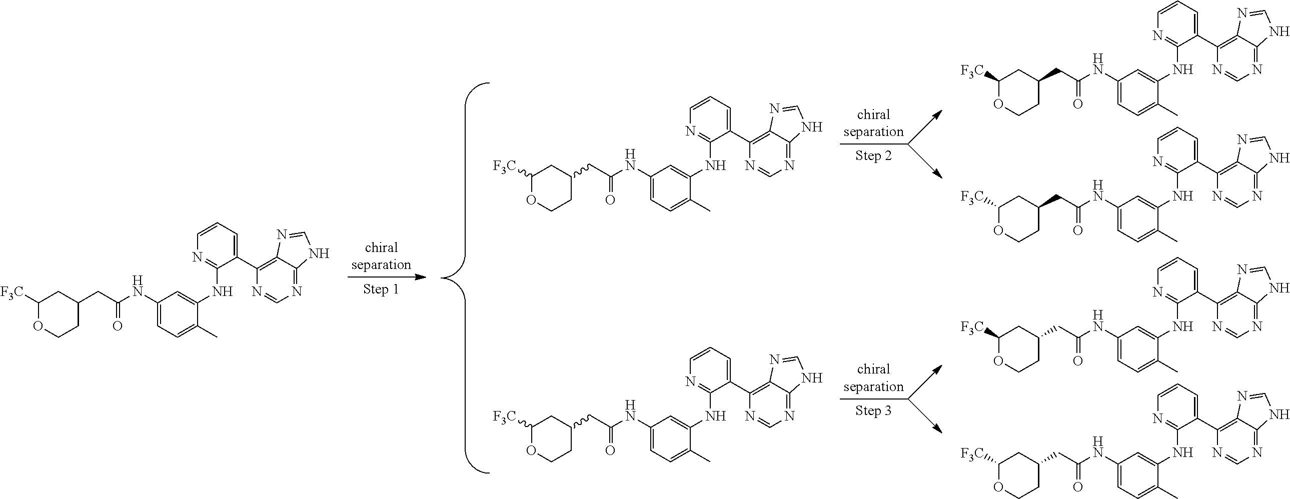

Step 1. A diastereomeric and racemic mixture of 4 isomers of N-(3-(3-(9H-purin-6-yl)pyridin-2-ylamino)-4-methylphenyl)-2-(2-(trifluoromethyl)tetrahydro-2H-pyran-4-yl)acetamide (228 mg, 0.45 mmol) was separated by chiral prep-HPLC (Instrument: SFC-80 (Waters) Column: AS 20×250 mm, 10 μm (Daicel); Mobile phase: CO₂/MeOH (0.5% methanol ammonia)=50/50) to give two mixtures of two isomers each (90 mg each).

Step 2. The first of the above mixtures was further separated by chiral prep-HPLC (Instrument: SFC-150 (Waters); Column: AS 20×250 mm, 10 μm (Daicel); Mobile phase: CO₂/MeOH (0.2% methanol ammonia)=65/35) to give separated isomers as yellow solids, arbitrarily assigned as N-(3-((3-(9H-purin-6-yl)pyridin-2-yl)amino)-4-methylphenyl)-2-((2R,4S)-2-(trifluoromethyl)tetrahydro-2H-pyran-4-yl)acetamide (45.8 mg,), ¹H NMR (400 MHZ, DMSO-d₆) δ 13.74 (s, 1H), 12.31 (s, 1H), 9.87 (s, 1H), 9.79 (d, J=7.5 Hz, 1H), 9.04 (s, 1H), 8.68 (s, 1H), 8.45 (d, J=1.8 Hz, 1H), 8.33 (dd, J=4.6, 1.8 Hz, 1H), 7.42-7.35 (m, 1H), 7.15 (d, J=8.3 Hz, 1H), 7.03 (dd, J=7.8, 4.7 Hz, 1H), 4.12-3.96 (m, 2H), 3.57-3.48 (m, 1H), 2.37 (d, J=18.8 Hz, 3H), 2.32 (d, J=5.2 Hz, 2H), 2.21-2.08 (m, 1H), 1.87-1.80 (m, 1H), 1.68-1.59 (m, 1H), 1.34-1.14 (m, 2H). MS (ESI) m/z 511.9 [M+H]⁺ *; and N-(3-((3-(9H-purin-6-yl)pyridin-2-yl)amino)-4-methylphenyl)-2-((2S,4S)-2-(trifluoromethyl)tetrahydro-2H-pyran-4-yl)acetamide (10.6 mg,), ¹H NMR (400 MHZ, DMSO-d₆) δ 13.84 (s, 1H), 12.28 (s, 1H), 9.89 (s, 1H), 9.77 (s, 1H), 9.07 (s, 1H), 8.72 (s, 1H), 8.45 (s, 1H), 8.34 (d, J=2.8 Hz, 1H), 7.39 (d, J=8.2 Hz, 1H), 7.16 (d, J=8.2 Hz, 1H), 7.04 (dd, J=7.8, 4.6 Hz, 1H), 4.32-4.20 (m, 1H), 3.89-3.68 (m, 2H), 2.64-2.54 (m, 1H), 2.50-2.43 (m, 2H), 2.40 (s, 3H), 1.92-1.72 (m, 2H), 1.69-1.55 (m, 1H), 1.45-1.33 (m, 1H). MS (ESI) m/z 512.0 [M+H]⁺

Step 3. The second of the above mixtures was further separated by chiral prep-HPLC (Instrument: SFC-80 (Thar, Waters); Column: AD 20×250 mm, 10 μm (Daicel); Mobile phase: CO₂/MeOH (0.2% methanol ammonia)=70/30) to give separated isomers as yellow solids, arbitrarily assigned as N-(3-((3-(9H-purin-6-yl)pyridin-2-yl)amino)-4-methylphenyl)-2-((2R,4R)-2-(trifluoromethyl)tetrahydro-2H-pyran-4-yl)acetamide (4.4 mg), ¹H NMR (400 MHZ, DMSO-d₆) δ 13.87 (s, 1H), 12.30 (s, 1H), 9.89 (s, 1H), 9.82 (s, 1H), 9.08 (s, 1H), 8.73 (s, 1H), 8.45 (s, 1H), 8.34 (dd, J=4.7, 1.8 Hz, 1H), 7.39 (d, J=8.0 Hz, 1H), 7.16 (d, J=8.2 Hz, 1H), 7.04 (dd, J=7.8, 4.6 Hz, 1H), 4.32-4.20 (m, 1H), 3.88-3.68 (m, 2H), 2.62-2.54 (m, 1H), 2.50-2.44 (m, 2H), 2.40 (s, 3H), 1.84-1.71 (m, 2H), 1.65-1.57 (m, 1H), 1.44-1.35 (m, 1H). MS (ESI) m/z 512.0 [M+H]+; and N-(3-((3-(9H-purin-6-yl)pyridin-2-yl)amino)-4-methylphenyl)-2-((2S,4R)-2-(trifluoromethyl)tetrahydro-2H-pyran-4-yl)acetamide (29.8 mg), ¹H NMR (400 MHZ, DMSO-d₆) δ 13.85 (s, 1H), 12.27 (s, 1H), 9.87 (s, 1H), 9.81 (s, 1H), 9.07 (s, 1H), 8.72 (s, 1H), 8.45 (s, 1H), 8.34 (d, J=2.8 Hz, 1H), 7.39 (d, J=7.9 Hz, 1H), 7.15 (d, J=8.5 Hz, 1H), 7.03 (dd, J=7.9, 4.7 Hz, 1H), 4.13-3.93 (m, 2H), 3.52 (t, J=11.0 Hz, 1H), 2.40 (s, 3H), 2.32 (d, J=5.1 Hz, 2H), 2.21-2.07 (m, 1H), 1.87-1.79 (m, 1H), 1.68-1.60 (m, 1H), 1.36-1.14 (m, 2H). MS (ESI) m/z 512.0 [M+H]⁺

### Example 147. Synthesis of N-(4-methyl-3-((3-(2-methyl-9H-purin-6-yl)pyridin-2-yl)amino)phenyl)-4-(trifluoromethyl)picolinamide (Compound IA-150)

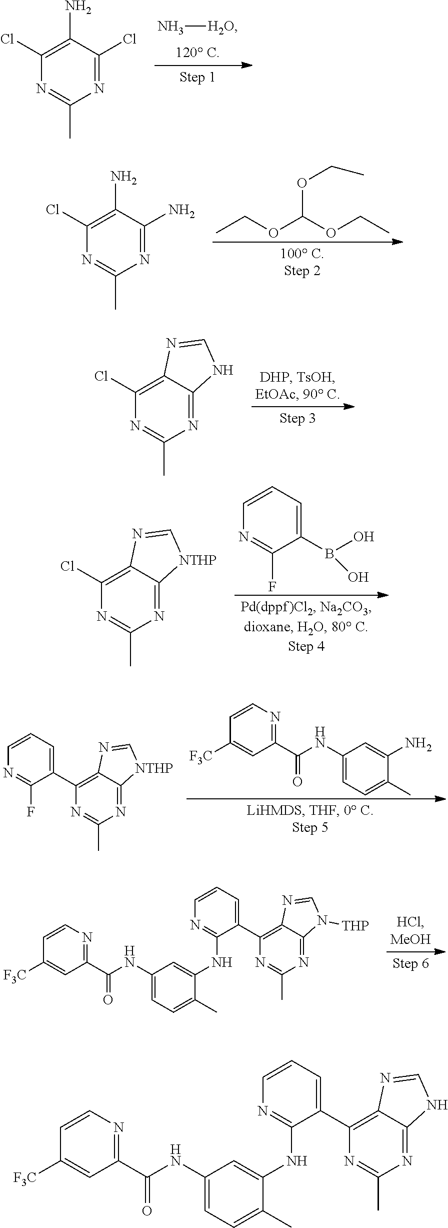

Step 1. A solution of 4,6-dichloro-2-methylpyrimidin-5-amine (1.0 g, 5.6 mmol) in ammonia hydrate (3 mL) was stirred at 120° C. for 4 hours. The reaction mixture was concentrated to afford 6-chloro-2-methylpyrimidine-4,5-diamine (810 mg, 5.13 mmol, 91%) as a yellow solid. MS (ESI) m/z 159.1 [M+H]⁺

Step 2. A solution of 6-chloro-2-methylpyrimidine-4,5-diamine (810 mg, 5.13 mmol) in triethoxymethane (10 mL) was stirred at 100° C. for 1.5 hours. The mixture was concentrated to afford 6-chloro-2-methyl-9H-purine (800 mg, 4.76 mmol, 93%) as a yellow solid. MS (ESI) m/z 169.1 [M+H]⁺

Step 3. To a solution of 6-chloro-2-methyl-9H-purine (680 mg, 4.05 mmol) and 4-methylbenzenesulfonic acid (35 mg, 0.202 mmol) in ethyl acetate (10 mL) was added 3,4-dihydro-2H-pyran (1020 mg, 12.1 mmol). The mixture was heated at 90° C. for 3 hours. The mixture was concentrated. The residue was purified by flash chromatography (dichloromethane/methanol=100/12) to give 6-chloro-2-methyl-9-(tetrahydro-2H-pyran-2-yl)-9H-purine (880 mg, 3.49 mmol, 86%) as a yellow solid. MS (ESI) m/z 255.1 [M+H]⁺

Step 4. A solution of 6-chloro-2-methyl-9-(tetrahydro-2H-pyran-2-yl)-9H-purine (280 mg, 1.11 mmol), (2-fluoropyridin-3-yl)boronic acid (211 mg, 1.50 mmol), 1,1′-bis(diphenylphosphino)ferrocene-palladium(II)dichloride dichloromethane complex (90 mg, 0.111 mmol) and sodium carbonate (235 mg, 2.22 mmol) in water (5 mL) and dioxane (15 mL) was stirred at 80° C. for 2 hours under argon. The reaction mixture was cooled. The organic layer was concentrated and purified by flash chromatography (dichloromethane/methanol=100/6) to give 6-(2-fluoropyridin-3-yl)-2-methyl-9-(tetrahydro-2H-pyran-2-yl)-9H-purine (320 mg, 1.02 mmol, 92%) as a yellow oil. MS (ESI) m/z 314.1 [M+H]⁺

Step 5. To a solution of N-(3-amino-4-methylphenyl)-4-(trifluoromethyl)picolinamide (57 mg, 0.1917 mmol), 6-(2-fluoropyridin-3-yl)-2-methyl-9-(tetrahydro-2H-pyran-2-yl)-9H-purine (60 mg, 0.1917 mmol) in tetrahydrofuran (8 mL) was added lithium hexamethyldisilazide (1.8 mL, 2.88 mmol, 1.6 M in tetrahydrofuran) at 0° C. slowly. The mixture was stirred at 0° C. for 1 hour. The reaction was quenched with ammonium chloride solution (30 mL) at 0° C. and extracted with dichloromethane (50 mL×3) The combined organic layers were washed with brine (50 mL), dried over anhydrous sodium sulfate, and filtered. The filtrate was concentrated under reduced pressure to give N-(4-methyl-3-((3-(2-methyl-9-(tetrahydro-2H-pyran-2-yl)-9H-purin-6-yl)pyridin-2-yl)amino)phenyl)-4-(trifluoromethyl)picolinamide (30 mg, 27%) as a yellow solid. MS (ESI) m/z 589.2 [M+H]⁺

Step 6. A solution of N-(4-methyl-3-((3-(2-methyl-9-(tetrahydro-2H-pyran-2-yl)-9H-purin-6-yl)pyridin-2-)yl)amino)phenyl)-4-(trifluoromethyl)picolinamide (30 mg, 0.051 mmol) in hydrochloric acid (3 M in methanol, 5 mL) was stirred at room temperature for 1 hour. The mixture was concentrated. The residue was purified by prep-HPLC (Column: Waters Xbridge 150×25 mm, 5 μm; Mobile phase: water (0.05% ammonia hydroxide)/acetonitrile, 30%-80%, 10 min) to give N-(4-methyl-3-((3-(2-methyl-9H-purin-6-yl)pyridin-2-yl)amino)phenyl)-4-(trifluoromethyl)picolinamide (12 mg, 0.0238 mmol, 46%) as a yellow solid. ¹H NMR (400 MHZ, DMSO-d₆) δ 13.35 (s, 1H), 11.81 (d, J=13.2 Hz, 1H), 10.78 (s, 1H), 9.67 (d, J=11.6 Hz, 1H), 9.03 (d, J=5.2 Hz, 1H), 8.61-8.56 (m, 2H), 8.37-8.31 (m, 1H), 8.30-. 8.29 (m, 1H), 8.10-8.07 (m, 1H), 7.57-7.51 (m, 1H), 7.26-7.22 (m, 1H), 7.03-7.00 (m, 1H), 2.93 (s, 3H), 2.44 (s, 3H). MS (ESI) m/z 504.8 [M+H]⁺

### Example 148. Synthesis of N-(3-(3-(2-fluoro-9H-purin-6-yl)pyridin-2-ylamino)-4-methylphenyl)-4-(trifluoromethyl)picolinamide (Compound IA-151)

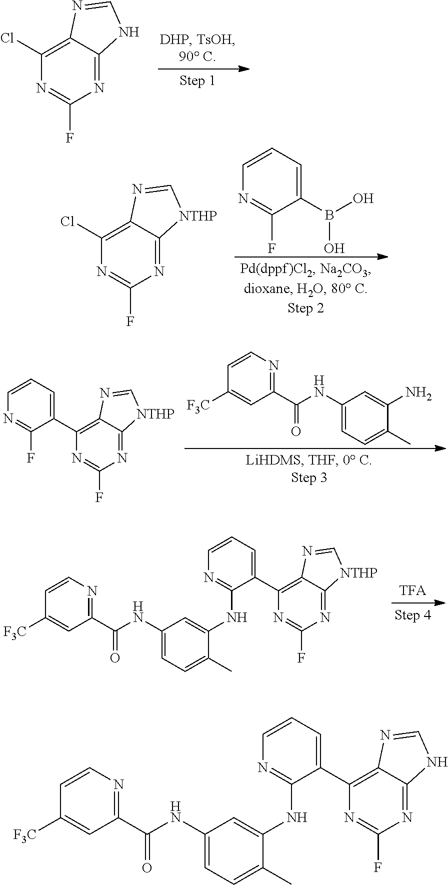

Step 1. A suspension of 6-chloro-2-fluoro-9H-purine (500 mg, 2.9 mmol) and 4-methylbenzenesulfonic acid (10 mg, 0.058 mmol) in ethyl acetate (15 mL) was treated with 3,4-dihydro-2H-pyran (731 mg, 8.7 mmol). The mixture was heated at 90° C., and the solid slowly dissolved over 3 hours. The mixture was concentrated. The residue was purified by flash chromatography (petroleum ether/ethyl acetate=5/2) to give 6-chloro-2-fluoro-9-(tetrahydro-2H-pyran-2-yl)-9H-purine (650 mg, 2.53 mmol, 87%) as a white solid. MS (ESI) m/z 173.1 [M−84+H]⁺

Step 2. A solution of 6-chloro-2-fluoro-9-(tetrahydro-2H-pyran-2-yl)-9H-purine (400 mg, 1.56 mmol), 2-fluoropyridin-3-ylboronic acid (286 mg, 2.03 mmol), 1,1′-bis(diphenylphosphino)ferrocene-palladium(II) dichloride dichloromethane complex (82 mg, 0.16 mmol) and sodium carbonate (496 mg, 4.68 mmol) in water (6 mL) and dioxane (20 mL) was stirred at 80° C. for 3 hours under argon. The reaction mixture was cooled. The organic layer was concentrated and purified by flash chromatography (ethyl acetate in petroleum ether from 30% to 50%) to give 2-fluoro-6-(2-fluoropyridin-3-yl)-9-(tetrahydro-2H-pyran-2-yl)-9H-purine (405 mg, 1.28 mmol, 82%) as a yellow solid. MS (ESI) m/z 234.1 [M−84+H]⁺

Step 3. To a solution of N-(3-amino-4-methylphenyl)-4-(trifluoromethyl)picolinamide (375 mg, 1.27 mmol), 2-fluoro-6-(2-fluoropyridin-3-yl)-9-(tetrahydro-2H-pyran-2-yl)-9H-purine (405 mg, 1.27 mmol) in tetrahydrofuran (20 mL) was added lithium hexamethyldisilazide (4 mL, 6.35 mmol, 1.6 M in tetrahydrofuran) at 0° C. slowly. The mixture was stirred at 0° C. for 1 hour. The reaction was quenched with saturated ammonium chloride solution (20 mL) at 0° C. and extracted with dichloromethane (60 mL×3). The combined organic layers were washed with brine (50 mL), dried over anhydrous sodium sulfate and filtered. The filtrate was concentrated under reduced pressure. The crude residue was purified by flash chromatography (ethyl acetate/petroleum ether=95/100) to give N-(3-(3-(2-fluoro-9-(tetrahydro-2H-pyran-2-yl)-9H-purin-6-yl)pyridin-2-ylamino)-4-methylphenyl)-4-(trifluoromethyl)picolinamide (120 mg) as a yellow solid. MS (ESI) m/z 592.7 [M+H]⁺

Step 4. A solution of N-(3-(3-(2-fluoro-9-(tetrahydro-2H-pyran-2-yl)-9H-purin-6-yl)pyridin-2-ylamino)-4-methylphenyl)-4-(trifluoromethyl)picolinamide (120 mg, 0.2 mmol) in trifluoroacetic acid (3 mL) was stirred at 20° C. for 1 hour. The reaction mixture was concentrated and purified by prep-HPLC (Column: BOSTON pHlex ODS 10 μm, 21.2×250 mm, 120 Å; Mobile phase: acetonitrile/0.1% ammonium bicarbonate) to afford N-(3-(3-(2-fluoro-9H-purin-6-yl)pyridin-2-ylamino)-4-methylphenyl)-4-(trifluoromethyl)picolinamide (10.3 mg, 0.02 mmol, 10%) as a yellow solid. ¹H NMR (400 MHZ, DMSO-d₆) δ 14.00 (s, 1H), 11.31 (s, 1H), 10.67 (s, 1H), 9.73 (s, 1H), 9.04 (d, J=5.0 Hz, 1H), 8.80 (s, 1H), 8.73 (s, 1H), 8.41 (dd, J=4.7, 1.8 Hz, 1H), 8.35 (s, 1H), 8.09 (d, J=4.7 Hz, 1H), 7.56 (d, J=8.1 Hz, 1H), 7.26 (d, J=8.3 Hz, 1H), 7.07 (dd, J=7.9, 4.7 Hz, 1H), 2.43 (s, 3H). MS (ESI) m/z 508.8 [M+H]⁺

### Example 149. Synthesis of N-(4-methyl-3-(6-methyl-3-(9H-purin-6-yl)pyridin-2-ylamino)phenyl)-4-(trifluoromethyl)picolinamide (Compound IA-152)

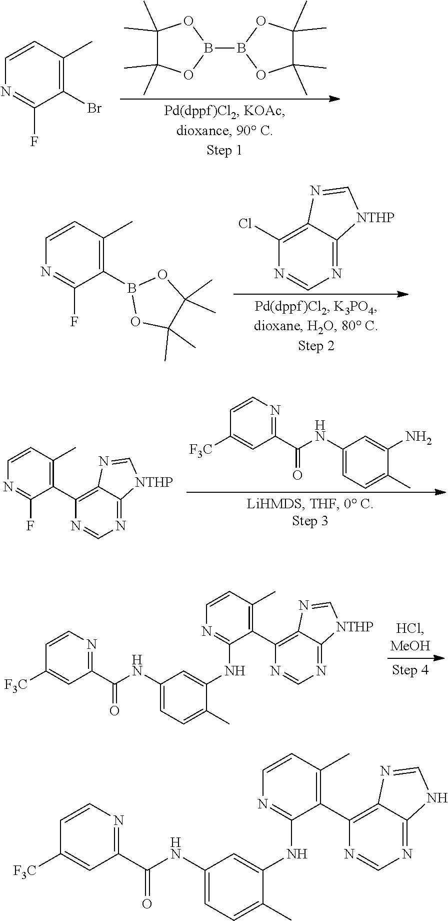

Step 1. A solution of 3-bromo-2-fluoro-4-methylpyridine (500 mg, 2.63 mmol), bis(pinacolato)diboron (1.34 g, 5.26 mmol), 1,1′-bis(diphenylphosphino)ferrocene-palladium(II) (192 mg, 0.26 mmol), and potassium acetate (773 mg, 7.89 mmol) in dioxane (15 mL) was stirred at 90° C. for 16 hours under argon to afford 2-fluoro-4-methyl-3-(4,4,5,5-tetramethyl-1,3,2-dioxaborolan-2-yl)pyridine. The mixture was used without further purification. MS (ESI) m/z 238.2 [M+H]⁺

Step 2. To a solution of 2-fluoro-4-methyl-3-(4,4,5,5-tetramethyl-1,3,2-dioxaborolan-2-yl)pyridine (623 mg, 2.63 mmol) in dioxane (15 mL) and water (3 mL) were added 6-chloro-9-(tetrahydro-2H-pyran-2-yl)-9H-purine (417 mg, 1.75 mmol), potassium phosphate tribasic (1.11 g, 5.25 mmol), and 1,1′-bis(diphenylphosphino)ferrocene-palladium(II) (124 mg, 0.17 mmol). The reaction mixture was stirred at 80° C. under argon for 16 hours. The reaction mixture was cooled. The organic layer was concentrated and purified by flash chromatography (ethyl acetate in petroleum ether from 30% to 90%) to give 6-(2-fluoro-4-methylpyridin-3-yl)-9-(tetrahydro-2H-pyran-2-yl)-9H-purine (250 mg, 46%) as a yellow solid. MS (ESI) m/z 314.1 [M+H]⁺

Step 3. To a solution of N-(3-amino-4-methylphenyl)-4-(trifluoromethyl)picolinamide (235 mg, 0.80 mmol) and 6-(2-fluoro-4-methylpyridin-3-yl)-9-(tetrahydro-2H-pyran-2-yl)-9H-purine (250 mg, 080 mmol) in tetrahydrofuran (20 mL) was added lithium hexamethyldisilazide (2.5 mL, 4.0 mmol, 1.6 M in tetrahydrofuran) at 0° C. slowly. The mixture was stirred at 0° C. for 1 hour. The reaction was quenched with saturated ammonium chloride solution (20 mL) at 0° C. and extracted with dichloromethane (50 mL×3). The combined organic layers were washed with brine (50 mL), dried over anhydrous sodium sulfate and filtered. The filtrate was concentrated under reduced pressure. The residue was purified by flash chromatography (methanol/dichloromethane=5/100) to give N-(4-methyl-3-(6-methyl-3-(9-(tetrahydro-2H-pyran-2-yl)-9H-purin-6-yl)pyridin-2-ylamino)phenyl)-4-(trifluoromethyl)picolinamide (100 mg, 42%) as a yellow solid. MS (ESI) m/z 589.2 [M+H]⁺

Step 4. To a solution of N-(4-methyl-3-((4-methyl-3-(9-(tetrahydro-2H-pyran-2-yl)-9H-purin-6-yl)pyridin-2-yl)amino)phenyl)-4-(trifluoromethyl)picolinamide (100 mg, 0.17 mmol) in methanol (10 mL) was added hydrochloric acid (3 M in methanol, 5 mL) at 25° C., and stirred for 1 hour. The reaction mixture was concentrated and diluted with methanol (10 mL). The pH was adjusted to 8 with 7 N ammonia in methanol solution. The mixture concentrated under reduced pressure, diluted with methanol (10 mL), and filtered. The solid was suspended in water (50 mL) and lyophilized under vacuum to afford N-(4-methyl-3-(6-methyl-3-(9H-purin-6-yl)pyridin-2-ylamino)phenyl)-4-(trifluoromethyl)picolinamide (52.5 mg, 61%) as a yellow solid. ¹H NMR (400 MHz, DMSO-d₆) δ 10.59 (s, 1H), 9.07 (s, 1H), 9.02-9.01 (d, J=4.8 Hz, 1H), 8.65 (S, 1H), 8.32 (s, 1H), 8.18 (s, 1H), 8.12-8.11 (d, J=5.2 Hz, 1H), 8.08-8.07 (d, J=4.8 Hz, 1H), 7.91 (s, 1H), 2.08 (s, 3H), 2.05 (s, 3H). MS (ESI) m/z 505.2 [M+H]⁺

### Example 150. Synthesis of N-(3-((6-methoxy-3-(9H-purin-6-yl)pyridin-2-yl)amino)-4-methylphenyl)-4-(trifluoromethyl)picolinamide (Compound IA-153)

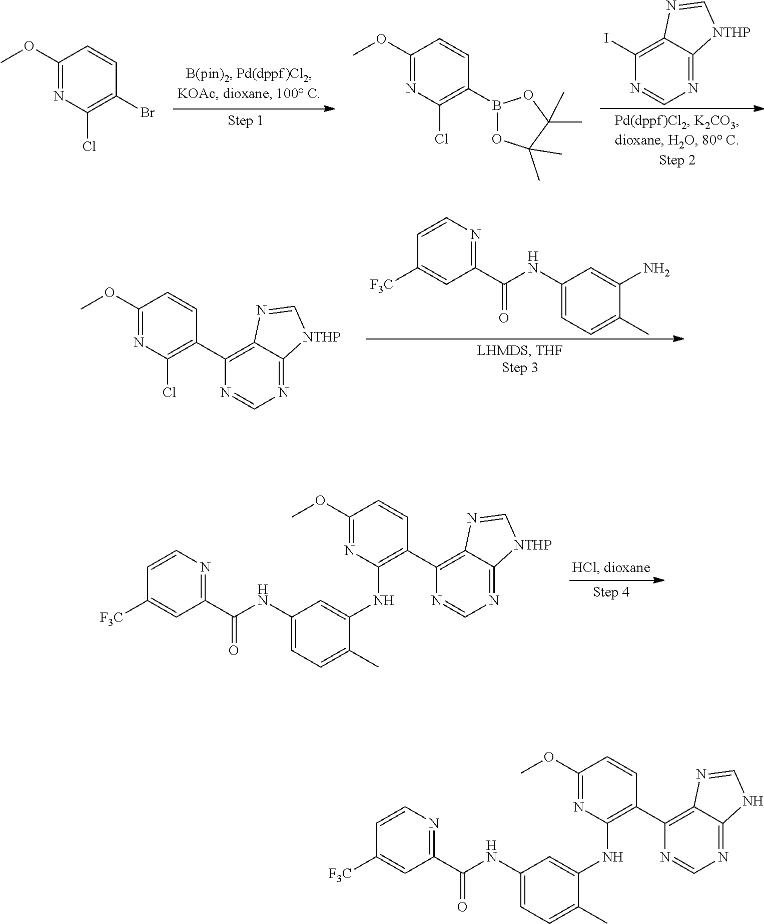

Step 1. A solution of 3-bromo-2-chloro-6-methoxypyridine (1 g, 4.54 mmol), [1,1′-bis(diphenylphosphino)ferrocene]dichloropalladium(II) (665 mg, 0.90 mmol), bis(pinacolato)diboron (1.38 g, 9.08 mmol) and potassium acetate (1.34 g, 13.62 mmol) in dioxane (10 mL) was stirred at 100° C. for 2 hours. The mixture was poured into water (10 mL) and extract with ethyl acetate (20 mL×3). The combined organic phases were concentrated and purified by flash chromatography (petroleum ether/ethyl acetate=4/1) to give crude 2-chloro-6-methoxy-3-(4,4,5,5-tetramethyl-1,3,2-dioxaborolan-2-yl)pyridine (1.5 g, 4.54 mmol, 99%) as a white solid. MS (ESI) m/z 270.1[M+H]⁺

Step 2. A solution of 2-chloro-6-methoxy-3-(4,4,5,5-tetramethyl-1,3,2-dioxaborolan-2-yl)pyridine (500 mg, 1.85 mmol), 6-iodo-9-(tetrahydro-2H-pyran-2-yl)-9H-purine (610 mg, 1.85 mmol), [1,1′-bis(diphenylphosphino)ferrocene]dichloropalladium(II) (270 mg, 0.37 mmol) and potassium carbonate (767 mg, 5.55 mmol) in dioxane (5 mL) and water (2 mL) was stirred at 80° C. for 3 hours. The reaction was poured into water (20 mL) and extracted with ethyl acetate (20 mL×3). The organic phase was concentrated and purified by flash chromatography (petroleum ether/ethyl acetate=2/1) to give 6-(2-chloro-6-methoxypyridin-3-yl)-9-(tetrahydro-2H-pyran-2-yl)-9H-purine (250 mg, 0.72 mmol, 40%) as a brown solid. MS (ESI) m/z 346.2 [M+H]⁺

Step 3. To a solution of 6-(2-chloro-6-methoxypyridin-3-yl)-9-(tetrahydro-2H-pyran-2-yl)-9H-purine (250 mg, 0.72 mmol) and N-(3-amino-4-methylphenyl)-4-(trifluoromethyl)picolinamide (213 mg, 0.72 mmol) in tetrahydrofuran (5 mL) was added lithium hexamethyldisilazide (1 M, 3.6 mL, 3.6 mmol) at 0° C., and then the mixture was stirred at 0° C. for 1 hour. The reaction was quenched with water (10 mL) and extracted with dichloromethane (30 mL×3). The combined organic layers were dried over anhydrous sodium sulfate, filtered and concentrated. The crude product was purified by flash chromatography (petroleum ether/ethyl acetate=1/2) to give N-(3-((6-methoxy-3-(9-(tetrahydro-2H-pyran-2-yl)-9H-purin-6-yl)pyridin-2-yl)amino)-4-methylphenyl)-4-(trifluoromethyl)picolinamide (200 mg, 0.33 mmol, 45%) as a brown solid. MS (ESI) m/z 605.3 [M+H]⁺

Step 4. A solution of N-(3-((6-methoxy-3-(9-(tetrahydro-2H-pyran-2-yl)-9H-purin-6-yl)pyridin-2-yl)amino)-4-methylphenyl)-4-(trifluoromethyl)picolinamide (20 mg, 0.033 mmol) in hydrochloric acid (4 N in dioxane, 2 mL) was stirred at room temperature overnight. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to give N-(3-((6-methoxy-3-(9H-purin-6-yl)pyridin-2-yl)amino)-4-methylphenyl)-4-(trifluoromethyl)picolinamide (3.0 mg, 0.006 mmol, 18%) as a yellow solid. ¹H NMR (400 MHZ, CD₃OD) δ 13.73 (s, 1H), 12.87 (s, 1H), 10.67 (s, 1H), 9.91 (d, J=12.4 Hz, 1H), 9.08 (d, J=1.6 Hz, 1H), 9.03 (d, J=4.8 Hz, 1H), 8.99 (s, 1H), 8.63 (s, 1H), 8.34 (s, 1H), 8.09 (d, J=5.2 Hz, 1H), 7.40 (t, J=7.6 Hz, 1H), 7.27 (d, J=8.0 Hz, 1H), 6.46 (d, J=8.8 Hz, 1H), 3.95 (s, 3H), 2.54 (s, 3H). MS (ESI) m/z 521.1 [M+H]⁺

### Example 151. Biological Activity of Exemplary Compounds

Retroviral Production: The BRAF-KIAA1549 fusion mutant was subcloned into pMXs-IRES-Blasticidin (RTV-016, Cell Biolabs, San Diego, CA). Retrovirus was produced by transfection of HEK 293T cells with the retroviral BRAF mutant expression vector pMXs-IRES-Blasticidin (RTV-016, Cell Biolabs), pCMV-Gag-Pol vector and pCMV-VSV-G-Envelope vector. Briefly, HEK 293T cells were plated in Poly-D-lysine coated 6-well plate (2.5×10⁵ cells per well) and incubated overnight. The next day, retroviral plasmids (1 μg of BRAF-KIAA fusion mutant, 0.32 μg of pCMV-Gag-Pol and 0.165 μg pCMV-VSV-G) were mixed in 300 μL of Optimem (31985, Life Technologies). The mixture was incubated at room temperature for 5 minutes and then added to Optimem containing transfection reagent Lipofectamine (11668, Invitrogen) and incubated for 20 minutes. The mixture was then added dropwise to HEK 293T cells. The next day the medium was replaced with fresh culture medium and retrovirus was harvested at 24 hours.

Generation of BRAF-KIAA1549 fusion stable cell line: BaF3 cells were plated in V-bottom 96 well plate (COSTAR #3894) in 80 μL of RPMI/10% FBS and 0.01 ng/ml mouse IL-3; supplemented with 8 μg/mL polybrene (4×10⁴ cells/well). Cells were transduced with 20 μL of BRAF-KIAA1549 fusion viral supernatant by centrifuging for 30 minutes at 1000 rpm. Cells were placed in a 37° C. incubator overnight. Next day 100 μL of RPMI/10% FBS and 0.01 ng/ml mouse IL-3 was added. After 24 hours, 100 μL of cells were transferred to a 24 well plate containing 900 μL of RPMI/10% FBS and 15 μg/mL Blasticidin. Contents of the 24 well plate were sampled for cell viability via CellTiterGlo (Promega) over a 2-week period. Cells that were observed to have greater than 1-fold viability over day of plating were expanded into T25 flasks for cell banking and Sanger sequence confirmation.

Assay for cell proliferation: BaF3 BRAF-KIAA1549 fusion cells were resuspended at 1.5×10⁵c/mL in RPMI containing 10% Heat Inactivated FBS, 1% L-glutamine and dispensed in duplicate (7×10⁴c/well) into 384 well plates. To determine the effect of compound on cell proliferation, BaF3 BRAF-KIAA1549 fusion cells were incubated for 96 hours in the presence of vehicle control (DMSO) or compound at varying concentrations. Inhibition of cell growth was determined by luminescent quantification (Envision by Perkin Elmer) of intracellular ATP content using CellTiterGlo (Promega), according to the protocol provided by the manufacturer. To determine the IC₅₀ values, the vehicle-treated cells were normalized as viable cells and analyzed using the CDD Vault, Collaborative Drug Discovery, Burlingame, CA (the Levenberg-Marquardt algorithm; Levenberg, K., 1994; Marquardt, D., 1963).

Tables A1 and A2 assign each compound a code for potency in the BaF3 BRAF-KIAA1549 fusion cell proliferation assay: A, B, C, or D. According to the code, A represents an IC₅₀ value <20 nM; B represents an IC₅₀ value ≥20 nM and <100 nM; C represents an IC₅₀ value ≥100 nM and <500 nM; and D represents an IC₅₀ value ≥500 nM.

TABLE A1

| Compound No. | BaF3 BRAF-KIAA1549 Fusion |
|---|---|
| I-2 | A |
| I-14 | A |
| I-16 | A |
| I-17 | B |
| I-18 | B |
| I-19 | A |
| I-20 | B |
| I-21 | B |
| I-22 | A |
| I-26 | C |
| I-27 | B |
| I-28 | D |
| I-29 | B |
| I-30 | A |
| I-31 | A |
| I-32 | A |
| I-33 | B |
| I-34 | B |
| I-35 | B |
| I-36 | A |
| I-37 | B |
| I-38 | B |
| I-39 | D |
| I-40 | D |
| I-41 | A |
| I-42 | A |
| I-43 | A |
| I-47 | A |
| I-48 | D |
| I-49 | A |
| I-50 | B |
| I-51 | D |
| I-52 | B |
| I-53 | B |
| I-54 | B |
| I-55 | A |
| I-56 | D |
| I-57 | D |
| I-58 | B |
| I-59 | C |
| I-60 | A |
| I-61 | B |
| I-62 | A |
| I-63 | C |
| I-64 | A |
| I-65 | A |
| I-66 | A |
| I-67 | B |
| I-68 | B |
| I-69 | B |
| I-70 | B |
| I-71 | B |
| I-72 | A |
| I-74 | B |
| I-75 | B |
| I-76 | D |
| I-77 | A |
| I-78 | B |
| I-79 | C |
| I-80 | A |
| I-81 | A |
| I-82 | A |
| I-83 | D |
| I-89 | D |
| II-3 | A |

TABLE A2

| Compound No. | BaF3 BRAF-KIAA1549 Fusion |
|---|---|
| IA-90 | B |
| IA-91 | B |
| IA-92 | B |
| IA-93 | B |
| IA-94 | C |
| IA-95 | A |
| IA-96 | B |
| IA-97 | A |
| IA-98 | A |
| IA-99 | B |
| IA-100 | B |
| IA-101 | A |
| IA-102 | C |
| IA-103 | A |
| IA-104 | A |
| IA-105 | B |
| IA-106 | A |
| IA-107 | A |
| IA-108 | C |
| IA-109 | A |
| IA-110 | B |
| IA-111 | A |
| IA-112 | C |
| IA-113 | A |
| IA-114 | A |
| IA-115 | A |
| IA-116 | A |
| IA-117 | C |
| IA-118 | B |
| IA-119 | C |
| IA-120 | B |
| IA-121 | D |
| IA-122 | B |
| IA-123 | B |
| IA-124 | B |
| IA-125 | B |
| IA-126 | A |
| IA-127 | A |
| IA-128 | A |
| IA-129 | A |
| IA-130 | A |
| IA-131 | D |
| IA-132 | D |
| IA-133 | D |
| IA-134 | D |
| IA-135 | D |
| IA-136 | D |
| IA-137 | A |
| IA-138 | A |
| IA-139 | A |
| IA-140 | A |
| IA-141 | C |
| IA-142 | C |
| IA-143 | C |
| IA-144 | C |
| IA-145 | A |
| IA-146 | B |
| IA-147 | B |
| IA-148 | B |
| IA-149 | B |
| IA-150 | D |
| IA-151 | D |
| IA-152 | D |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I′): 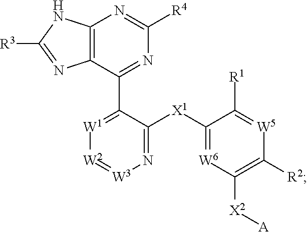
or a pharmaceutically acceptable salt or stereoisomer thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, C₂-C₆ alkynyl, or C₁-C₆ alkoxyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
R² is H, halogen, C₁-C₆ alkyl, C₂-C₆ alkenyl, or C₂-C₆ alkynyl;
R³ is H, halogen, C₁-C₆ alkyl, C₂-C₆ alkenyl, C₂-C₆ alkynyl, or C₁-C₆ alkoxyl,
wherein the C₁-C₆ alkoxyl is optionally substituted with one or more OH, NH₂, NH(C₁-C₆ alkyl), or N(C₁-C₆ alkyl)₂;
R⁴ is H, halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is absent, C₂-C₆ alkynyl, —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A};
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH(C₁-C₆ alkyl), or N(C₁-C₆ alkyl)₂ is optionally substituted with one or more R^{A1}; and
each R^{A1} independently is halogen, cyano, OH, NH₂, NH(C₁-C₆ alkyl), N(C₁-C₆ alkyl)₂, C₁-C₆ alkyl, C₂-C₆ alkenyl, or C₂-C₆ alkynyl.

2. A compound of Formula (I): 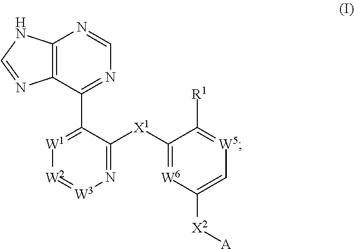
or a pharmaceutically acceptable salt or stereoisomer thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl;
W³ is N or CR^{W3};
R^{W3} is H, halogen, C₁-C₆ alkyl, C₂-C₆ alkenyl, or C₂-C₆ alkynyl;
X¹ is NR^{X1}, O, or S;
R^{X1} is H, C₁-C₆ alkyl, C₂-C₆ alkenyl, or C₂-C₆ alkynyl;
R¹ is halogen, C₁-C₆ alkyl, C₂-C₆ alkenyl, or C₂-C₆ alkynyl;
W⁵ is N or CR^{W5};
R^{W5} is H, halogen, C₁-C₆ alkyl, C₂-C₆ alkenyl, or C₂-C₆ alkynyl;
W⁶ is N or CR^{W6};
R^{W6} is H, C₁-C₆ alkyl, C₂-C₆ alkenyl, or C₂-C₆ alkynyl;
X² is absent, C₂-C₆ alkynyl, —NR^{X2}C(O)—*, —C(O)NR^{X2}—*, —NR^{X2}C(O)CH₂—*, or —C(O)NR^{X2}CH₂—*, wherein * denotes attachment to A;
R^{X2} is H, C₁-C₆ alkyl, C₂-C₆ alkenyl, or C₂-C₆ alkynyl;
A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}, and
each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, NH(C₁-C₆ alkyl), or N(C₁-C₆ alkyl)₂ is optionally substituted with one or more halogen or cyano.

3. The compound of any one of the preceding claims, wherein W¹ is CR^{W1}, W² is CR^{W2}, and W³ is CR^{W3}.

4. The compound of any one of the preceding claims, wherein R^{W1} is H, halogen, or C₁-C₆ alkyl.

5. The compound of any one of the preceding claims, wherein R^{W2} is H, or halogen.

6. The compound of any one of the preceding claims, wherein R^{W3} is H, or C₁-C₆ alkoxyl.

7. The compound of any one of the preceding claims, wherein W¹, W², and W³ each are CH.

8. The compound of any one of the preceding claims, wherein W¹ is N, W² is CR^{W2}, and W³ is CR^{W3}.

9. The compound of any one of the preceding claims, wherein W¹ is CR^{W1}, W² is CR^{W2}, and W³ is N.

10. The compound of any one of the preceding claims, wherein X¹ is NR^{X1}, NH, O or S.

11. The compound of any one of the preceding claims, wherein R¹ is halogen, or C₁-C₆ alkyl.

12. The compound of any one of the preceding claims, wherein R² is H, or C₁-C₆ alkyl.

13. The compound of any one of the preceding claims, wherein R³ is H, C₁-C₆ alkyl, or C₁-C₆ alkoxyl, wherein the C₁-C₆ alkoxyl is optionally substituted with one or more OH, NH₂, NH(C₁-C₆ alkyl), or N(C₁-C₆ alkyl)₂.

14. The compound of any one of the preceding claims, wherein R⁴ is H, halogen, or C₁-C₆ alkyl.

15. The compound of any one of the preceding claims, wherein W⁵ is N, or CR^{W5}.

16. The compound of any one of the preceding claims, wherein R^{W5} is H, halogen, or C₁-C₆ alkyl.

17. The compound of any one of the preceding claims, wherein W⁶ is N, or CR^{W6}.

18. The compound of any one of the preceding claims, wherein R^{W6} is H, halogen, or C₁-C₆ alkyl.

19. The compound of any one of the preceding claims, wherein X² is absent, C₂-C₆ alkynyl, —NR^{X2}C(O)—*, —C(O)NR^{X2}*, —NR^{X2}C(O)CH₂—*, or —C(O)NR^{X2}CH₂—*, wherein * denotes attachment to A.

20. The compound of any one of the preceding claims, wherein A is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R^{A}.

21. The compound of any one of the preceding claims, wherein A is

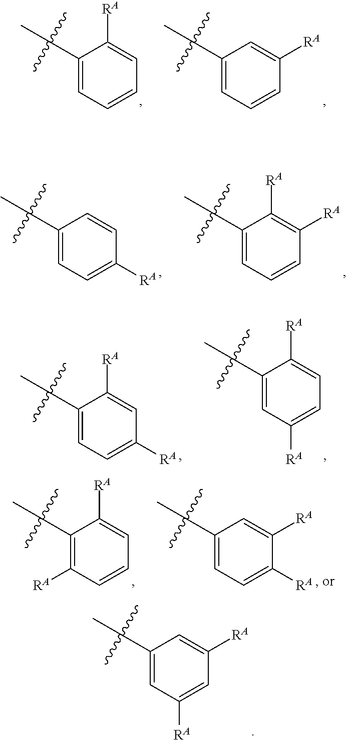

22. The compound of any one of the preceding claims, wherein each R^{A} independently is halogen, cyano, C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH₂, NH(C₁-C₆ alkyl), or N(C₁-C₆ alkyl)₂, wherein the C₁-C₆ alkyl, C₂-C₆ alkenyl, C₂-C₆ alkynyl, C₁-C₆ alkoxyl, C₃-C₈ cycloalkyl, 3- to 8-membered heterocycloalkyl, —O—(C₃-C₈ cycloalkyl), —O-(3- to 8-membered heterocycloalkyl), NH(C₁-C₆ alkyl), or N(C₁-C₆ alkyl)₂ is optionally substituted with one or more R^{A1}.

23. The compound of any one of the preceding claims, wherein each R⁴¹ independently is halogen, cyano, OH, NH₂, NH(C₁-C₆ alkyl), N(C₁-C₆ alkyl)₂, C₁-C₆ alkyl, C₂-C₆ alkenyl, or C₂-C₆ alkynyl.

24. The compound of any one of the preceding claims, being of Formula (II), (III-a), (III-b), (III-c), (III-d), (II-+), (II-f), (IV-a) or (IV-b): 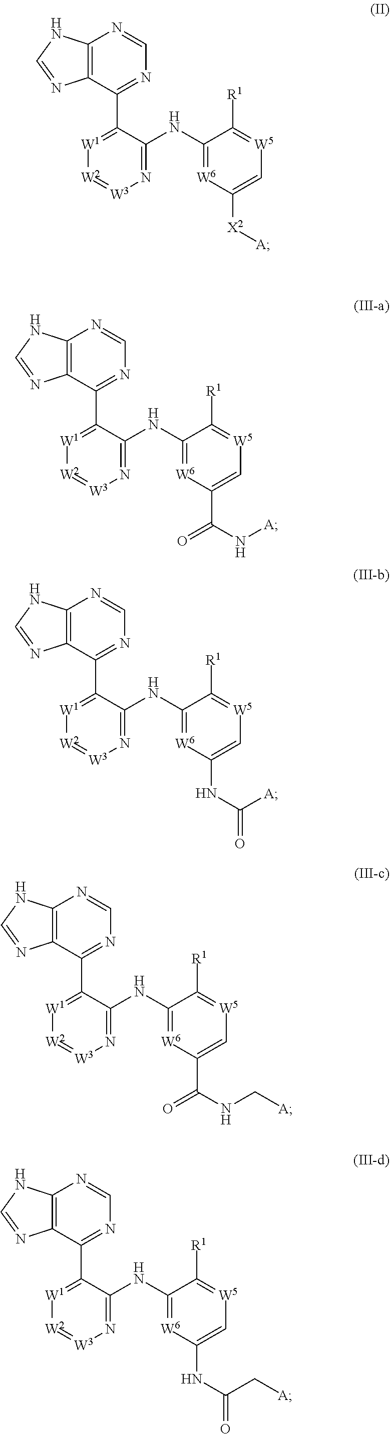 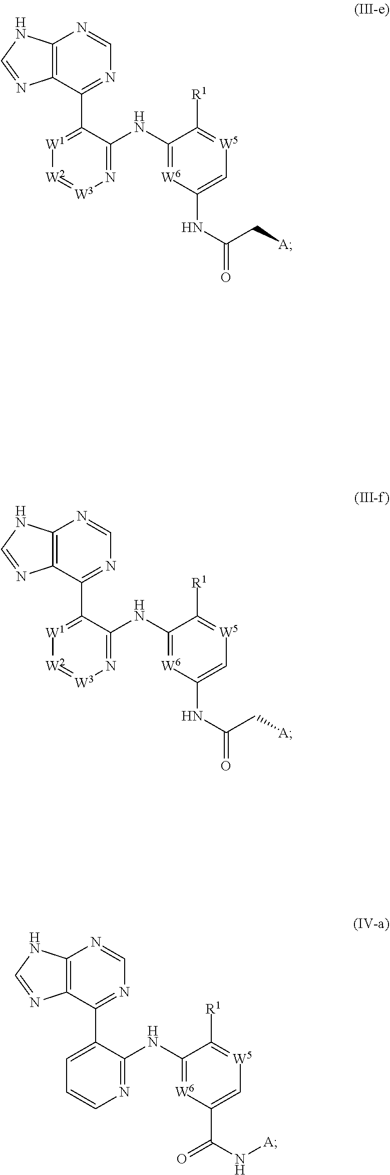 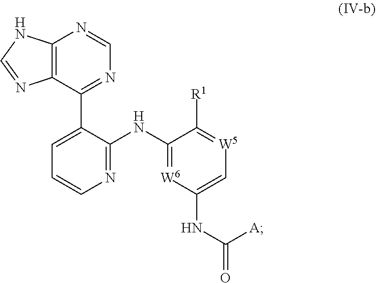
or a pharmaceutically acceptable salt or stereoisomer thereof.

25. The compound of any one of the preceding claims, being selected from the compounds described in Tables I, IA and II, or a pharmaceutically acceptable salt or stereoisomer thereof.

26. The compound of any one of the preceding claims, being selected from the compounds described in Table II and pharmaceutically acceptable salts and stereoisomers thereof.

27. A pharmaceutical composition comprising the compound of any one of the preceding claims and one or more pharmaceutically acceptable carriers or excipients.

28. A method of treating or preventing cancer in a subject, the method comprising administering to the subject a compound of any one of the preceding claims.

29. A compound of any one of the preceding claims for treating or preventing cancer in a subject.

30. Use of the compound of any one of the preceding claims in the manufacture of a medicament for treating or preventing cancer in a subject.

31. Use of the compound of any one of the preceding claims for treating or preventing cancer in a subject.

32. The method, compound, or use of any one of the preceding claims, wherein the cancer is characterized by at least one oncogenic mutation in the BRAF gene.

33. The method, compound, or use of any one of the preceding claims, wherein the cancer is characterized by at least one oncogenic variant of B-Raf.

34. The method, compound, or use of any one of the preceding claims, wherein the subject has at least one oncogenic mutation in the BRAF gene.

35. The method, compound, or use of any one of the preceding claims, wherein the subject has at least one tumor and/or cancerous cell that expresses an oncogenic variant of B-Raf.

36. The method, compound, or use of any one of the preceding claims, wherein the oncogenic mutation is a class II mutation or a class III mutation. Accordingly, in some embodiments, the oncogenic variant of B-Raf comprises a class II mutation.

37. The method, compound, or use of any one of the preceding claims, wherein the oncogenic variant of B-Raf can be any of the B-Raf variants put forth in Table 1b.

38. The method, compound, or use of any one of the preceding claims, wherein the cancer is a hematological cancer, solid cancer, melanoma, breast cancer, head and neck cancer, esophagogastric cancer, stomach and small intestine cancer, lung cancer, mesothelioma, hepatobiliary cancer, pancreatic cancer, kidney cancer, colorectal cancer, endometrial cancer, cervical cancer, ovarian cancer, bladder cancer, prostate cancer, soft tissue sarcoma, CNS and brain cancer, thyroid cancer, non-small cell lung cancer (NSCLC), colorectal cancer, melanoma, thyroid cancer, histiocytosis, small bowel cancer, gastrointestinal neuroendocrine cancer, carcinoma of unknown primary, non-melanoma skin cancer, prostate cancer, gastric cancer, non-Hodgkin's lymphoma, papillary thyroid carcinoma or glioblastoma.
